(19) United States
(12) Patent Application Publication
(10) Pub. No.: US 2026/0316976 A1
(43) Pub. Date: Oct. 15, 2026

(54) Systems, Methods, and Apparatuses for Producing and Packaging Medical Fluids

(71) Applicant: DEKA Products Limited Partnership, Manchester, NH (US)

(72) Inventors: Jason M. Overson, Manchester, NH (US); Robert Salisbury, Brookfield, NH (US); Billie J. Strong, Northfield, NH (US); Gillian R. Cohen, Kalamazoo, MI (US); Joseph P. Hochadel, Bow, NH (US); Abdullah M. A. E. Almutawa, Merrimack, NH (US); Robert H. Lawler, JR., Manchester, NH (US); Justin M. Whitney, Goffstown, NH (US); Bryson N. Halsey, Manchester, NH (US); Perry F. Craver, Gilford, NH (US); Ryan P. Wood, Bedford, NH (US); John J. Wight, JR., Manchester, NH (US); Robert Piccirillo, Salem, NH (US); Richard S. Babel, Webster, NH (US); Matthew Handley, Manchester, NH (US); Michael J. Haynes, Manchester, NH (US); Blake Lagasse, Manchester, NH (US); Caleb D. Kase, Manchester, NH (US); Kunj P. Patel, Manchester, NH (US); Mohammed Aun Siddiqui, San Carlos, CA (US); Stepan Vartanov, Manchester, NH (US); Samuel P. Rooney, Strafford, NH (US); Michael K. LeVasseur, Concord, NH (US); Avery X. Roy, Manchester, NH (US); Boon Ping Ng, Andover, MA (US); Xingpan Jin, Manchester, NH (US); Bruno L. Bertolo, Manchester, NH (US); Michael Quilici, Concord, NH (US); Andrew A. Schnellinger, Merrimack, NH (US); Kayli M. Hill, Loudon, NH (US); Peter A. Cajolet, Manchester, NH (US); Bayard D. Snow, Bow, NH (US); Thaddeus J. Hughes, Shirley, IL (US); Nicholas S. Wheaton, Dunbarton, NH (US); Rajesh G. Nagula, Nashua, NH (US); Michael G. Norris, Manchester, NH (US); Gregory J. Buitkus, North Andover, MA (US); Archie G. Milligan, Medford, MA (US); Priya Vijayakumar, Grand Rapids, MI (US); Stephen J. Narvesen, Bedford, NH (US); Rahul Dev Appapogu, Manchester, NH (US); Jacob S. Squibb, Nashua, NH (US); Dean Kamen, Bedford, NH (US)

(21) Appl. No.: 19/573,808

(22) Filed: Mar. 20, 2026

Related U.S. Application Data
(63) Continuation-in-part of application No. 19/223,918, filed on May 30, 2025.
(63) Continuation-in-part of application No. 19/223,870, filed on May 30, 2025.
(60) Provisional application No. 63/677,620, filed on Jul. 31, 2024.
(60) Provisional application No. 63/677,620, filed on Jul. 31, 2024.
(60) Provisional application No. 63/775,493, filed on Mar. 21, 2025.
(60) Provisional application No. 63/775,457, filed on Mar. 21, 2025.
(60) Provisional application No. 63/775,455, filed on Mar. 21, 2025.
(60) Provisional application No. 63/775,493, filed on Mar. 21, 2025.
(60) Provisional application No. 63/775,457, filed on Mar. 21, 2025.
(60) Provisional application No. 63/775,455, filed on Mar. 21, 2025.
(60) Provisional application No. 63/815,051, filed on May 30, 2025.
(60) Provisional application No. 63/904,709, filed on Oct. 24, 2025.
(60) Provisional application No. 64/011,358, filed on Mar. 20, 2026.

(51) Int. Cl. B65B 3/00; A61L 2/104; A61L 103/10; B65B 3/04; G06N 3/0464; G06T 7/00

(52) U.S. Cl. CPC B65B 3/003; A61L 2/104; B65B 3/045; G06N 3/0464; G06T 7/0002; A61L 2103/10; G06T 2207/10004; G06T 2207/20081; G06T 2207/30108

(57) ABSTRACT

An example dispenser of medical reservoirs may comprise a rigid frame defining a central fenestration. The dispenser may further comprise a shell coupled to the frame. The dispenser may further comprise a removable cover retained within the fenestration. The cover may include a panel with a port and a breathable microbial barrier extending across the port coupled to the panel. The frame, shell, and cover may define an interior dispenser volume. The dispenser may further comprise a frangible coupled to and at least partially covering exterior faces of the frame and the panel. The frangible may have an aperture aligned with the port. The frangible may be coupled to the exterior faces of the frame and the panel to form seals around the port, along the exterior face of the frame adjacent the fenestration, and along the exterior face of the panel adjacent the peripheral edge of the panel.

### CROSS-REFERENCE TO RELATED APPLICATIONS

The present application is a Continuation-in-Part of U.S. Utility application Ser. No. 19/223,918, entitled Systems, Methods, and Apparatuses for Producing and Packaging Medical Fluids, filed May 30, 2025 (Attorney Docket No. 00101.00509.AB781), which claims the benefit of U.S. Provisional Application Ser. No. 63/677,620, entitled System for and Method of Identifying an Object, filed Jul. 31, 2024 (Attorney Docket No. 00101.00507.AB522), and claims the benefit of U.S. Provisional Application Ser. No. 63/775,493, entitled Systems, Methods, and Apparatuses for Producing and Packaging Medical Fluids, filed Mar. 21, 2025 (Attorney Docket No. 00101.00465.AB670), and claims the benefit of U.S. Provisional Application Ser. No. 63/775,457, entitled Systems, Methods, and Apparatuses for Producing and Packaging Medical Fluids, filed Mar. 21, 2025 (Attorney Docket No. 00101.00336.AA849), and claims the benefit of U.S. Provisional Application Ser. No. 63/775,455, entitled Systems, Methods, and Apparatuses for Producing and Packaging Medical Fluids, filed Mar. 21, 2025 (Attorney Docket No. 00101.00335.AA829).

The present application is a Continuation-in-Part of U.S. Utility application Ser. No. 19/223,870, entitled Systems, Methods, and Apparatuses for Producing and Packaging Medical Fluids, filed May 30, 2025 (Attorney Docket No. 00101.00513.AB785), which claims the benefit of U.S. Provisional Application Ser. No. 63/677,620, entitled System for and Method of Identifying an Object, filed Jul. 31, 2024 (Attorney Docket No. 00101.00507.AB522), and claims the benefit of U.S. Provisional Application Ser. No. 63/775,493, entitled Systems, Methods, and Apparatuses for Producing and Packaging Medical Fluids, filed Mar. 21, 2025 (Attorney Docket No. 00101.00465.AB670), and claims the benefit of U.S. Provisional Application Ser. No. 63/775,457, entitled Systems, Methods, and Apparatuses for Producing and Packaging Medical Fluids, filed Mar. 21, 2025 (Attorney Docket No. 00101.00336.AA849), and claims the benefit of U.S. Provisional Application Ser. No. 63/775,455, entitled Systems, Methods, and Apparatuses for Producing and Packaging Medical Fluids, filed Mar. 21, 2025 (Attorney Docket No. 00101.00335.AA829).

The present application claims the benefit of U.S. Provisional Application Ser. No. 63/815,051, entitled Systems, Methods, and Apparatuses for Producing and Packaging Medical Fluids, filed May 30, 2025 (Attorney Docket No. 00101.00508.AB780).

The present application claims the benefit of U.S. Provisional Application Ser. No. 63/904,709, entitled Systems, Methods, and Apparatuses for Producing and Packaging Medical Fluids, filed Oct. 24, 2025 (Attorney Docket No. 00101.00544.TA008).

The present application claims benefit of U.S. Provisional Application Ser. No. 64/011,358, entitled Bag Vending Apparatuses and Related Methods, filed Mar. 20, 2026 (Attorney Docket No. 00101.00654.TA121), all of the above being incorporated by reference herein in their entireties.

### STATEMENT REGARDING FEDERALLY SPONSORED RESEARCH OR DEVELOPMENT

This invention was made with Government support under Agreement HHSO100201900017C, awarded by the U.S. Department of Health and Human Services. The Government has certain rights in the invention.

This invention was made with Government support under Agreement N00014-23-9-0001, awarded by The Office of Naval Research. The Government has certain rights in the invention.

### BACKGROUND

### Field of Disclosure

This disclosure relates to medical fluids. More specifically, this disclosure relates to the generation and packaging of medical fluids.

### Description of Related Art

Almost every hospitalized patient is administered saline or a saline based solution. As a result, the quantity of saline solution consumed is very large. More than a billion bags of saline are used per year in the US alone. Despite the demand, there are only a small number of different saline manufactures which provide this solution for the US market. Unfortunately, manufacturing challenges which limit production from one manufacturer can and do cause shortages of saline in the United States. Compounding the issue, these manufactures have uneven market share in regards to all bagged saline products. For instance, 50% of 250 ml or smaller saline bags are provided by a single manufacture. As a result, when such a manufacturer faces production problems, the impact on the availability of that particular type of bag is much greater.

Most recently, the media spotlight has been shown on delays caused in the wake of hurricane Maria which have led to a shortage of small volume saline bags. According to the American Society of Health-System Pharmacists, shortages for large volume bags and bags of saline for irrigation purposes also currently exist. An alternative means of producing medical fluid bags which may perhaps be locatable in the institution using the bag would be desirable.

### SUMMARY

In accordance with an example embodiment of the present disclosure, an example fluid dispensing cartridge for a medical fluid dispenser may comprise a housing assembly having a flow path extending therethrough. The cartridge may further comprise an irradiation transmissive barrier coupled to the housing assembly. The cartridge may further comprise an airflow channel extending through the housing assembly. The irradiation transmissive barrier may define at least a portion of the airflow channel. One end of the airflow channel may include at least one port guide. The cartridge may further comprise a dispensing sharp coupled to a hub within the airflow channel. The dispensing sharp may be in fluid communication with an output end of the flow path. The cartridge may further comprise a connection port disposed at an input end of the flow path having a plurality of sealing members in spaced relation about an exterior surface of the connection port. The cartridge may further comprise a frangible seal at a terminal end of the connection port. The cartridge may further comprise at least one filter disposed at an intermediate portion of the flow path.

In some embodiments, the dispensing sharp may be a pencil tipped sharp with a least one side port. In some embodiments, the dispensing sharp may be a pencil tipped sharp with two side ports disposed in opposition to one another. In some embodiments, the dispensing sharp may be a pencil tipped sharp with a gauge between 16-18. In some embodiments, the airflow channel may vary in cross-sectional area along a length of the airflow channel. In some embodiments, the irradiation transmissive barrier may be at least partially transparent to UVC light. In some embodiments, the irradiation transmissive barrier may be formed of fused quartz. In some embodiments, the irradiation transmissive barrier may be a tube of fused quartz. In some embodiments, the airflow channel may extend along a straight line path through the housing and has a round cross-sectional shape. In some embodiments, the connection port may have a first section having a first diameter and a second section having a second diameter smaller than the first. In some embodiments, the second section may extend to the terminal end of the connection port and at least one sealing member may be disposed on the exterior surface of each of the first second and second section. In some embodiments, the at least one filter may include a 0.2 μm filter. In some embodiments, the cartridge further comprises at least one filter support for each of the at least one filter. In some embodiments, the cartridge further may comprise filter support grates. A first filter support grate may be disposed on a downstream side of each of the at least one filter. A second filter support grate may be disposed on the upstream side of each of the at least one filter. In some embodiments, the filter may be captured between a first and second body of the housing assembly.

In accordance with another example embodiment of the present disclosure an example particulate inspection system for detecting and classifying contents of interest within a medical reservoir may comprise a cradle including a reservoir rest body and a reservoir retainer. The cradle may include a backlight. The rest body may be in an illumination field of the backlight and may include a pattern of light and dark regions. The system may further comprise an external illuminator including at least one light emitter. The system may further comprise a vision assembly disposed opposite the cradle. The vision assembly may include at least one imager having a field of view encompassing at least a portion of the cradle. The system may further comprise a controller in data communication with each of the at least one imager. The controller may be configured to command capture of a series of source images from each of the at least one imager, receive the source images, pre-process the source images into processed images, detect regions of interest within the processed images, and classify the regions of interest with a plurality of trained machine learning models. Each region of interest may be classified by a trained machine learning model of the plurality of trained machine learning models which is specific to the imager which captured the source image from which that region of interest was detected.

In some embodiments, the regions of interest are classified into at least a first content type and second content type based on the analysis of the regions of interest. In some embodiments, the controller may be configured generate a reservoir acceptability determination. The acceptability determination may be indicated as a failure if any contents of interest are classified as the second content type. In some embodiments, the first content type may be a gas bubble and the second content type may be anything other than a gas bubble. In some embodiments, the system may further comprise a rotary actuator coupled to the cradle. In some embodiments, the backlight may emit white light. In some embodiments, the pattern may be a chessboard pattern. In some embodiments, the reservoir retainer may be a set of IV bag port graspers actuatable between an open state and port retaining state. In some embodiments, the external illuminator may be an underlight. In some embodiments, the at least one light emitter may include a plurality of LEDs electrically coupled in series. In some embodiments, each of the at least one light emitter may be a collimated light emitter. In some embodiments, each of the at least one light emitter may emit light in a narrow beam angle less than 20°. In some embodiments, the external illuminator may include a polarizer. In some embodiments, each of the at least one imager may include a polarizing filter on a lens thereof. In some embodiments, the vision assembly may include a plurality of imagers. Each field of view of each imager may overlap the field of view of at least one other imager of the vision system. In some embodiments, a view axis each of the at least one imager may be oriented at an angle 30-35° from a plane extending perpendicular to a height axis of the cradle. In some embodiments, the controller may be further configured to assign each region of interest in each of the processed images to one or more track. Each of the one or more track may define a displacement path of an associated region of interest over the series of images. In some embodiments, for each track, the controller may be configured to extract an extracted portion of every processed image in which the associated region of interest is detected. The extracted portion may include the associated region of interest. Each of the plurality of trained machine learning models may be trained to classify regions of interest by analyzing the extracted portions of the processed images for each track. In some embodiments, each of the plurality of trained machine learning models may be a convolutional neural network. In some embodiments, the medical reservoir may be an IV bag.

In accordance with another example embodiment of the present disclosure, an example method of inspecting a medical reservoir may comprise placing the medical reservoir in a cradle. The method may further comprise providing a background on first side of the medical reservoir which contrasts between light and dark. The method may further comprise illuminating the medical reservoir with an illumination assembly separate from the cradle. The method may further comprise collecting at least one series of frames of the medical reservoir from at least one imager on a second side of the medical reservoir opposite the first. The method may further comprise processing each of the frames in the at least one series of frames into a series of processed frames. The method may further comprise detecting regions of interest within each of the processed frames of the series of processed frames. The method may further comprise associating each of the detected regions of interest with tracks. Each track may define a displacement path of a specific tracked region of interest over a plurality of processed frames. The method may further comprise classifying the specific tracked region of interest in each processed frame for each track with a specific trained machine learning model of a plurality of trained machine learning models. The specific trained machine learning model may be selected from the plurality of trained machine learning model based on the imager of the at least one imager which captured the series of frames the specific tracked region of interest was ultimately detected from.

In some embodiments, placing the medical reservoir in the cradle may comprise placing an IV bag in the cradle. In some embodiments, the method may further comprise agitating the medical reservoir. In some embodiments, agitating the medical reservoir may comprise rotating the medical reservoir through a series of rotational orientations specified in a motion profile. In some embodiments, providing the background may comprise placing a pattern of light and black regions on a rest panel of the cradle. In some embodiments, providing the background may comprise adjusting a backlight on the first side of the medical reservoir to provide a background which provides a temporal contrast between light and dark. In some embodiments, illuminating the medical reservoir may comprise directing collimated light at the medical reservoir from an underlight. In some embodiments, illuminating the medical reservoir may comprise directing polarized light at the medical reservoir. In some embodiments, the at least one imager may include a plurality of imagers each having a field of view which at least partially overlaps that of at least one other imager of the plurality of imagers. Collecting the at least one series of frames may comprise collecting a plurality of series of frames. Each plurality of series of frames may be captured from a respective imager of the plurality of imagers. In some embodiments, the method may further comprise providing a polarizing filter for each of the at least one imager. In some embodiments, processing each of the frames may comprise at least one of smoothing each of the frames, generating a foreground segmented image, generating a near edge filtered foreground segmented image based on an edge detection analysis of each frame, performing at least one kernel convolution, performing at least one morphological transformation, and removing pixel clusters having yielding a contour outside of a predefined size range. In some embodiments, detecting regions of interest within each of the processed frames may comprise generating a bounding box around each pixel cluster of interest remaining in the processed frames. In some embodiments, associating each of the detected regions of interest with tracks may comprise determining track predictions for each region of interest in a starting frame and matching the track predictions to detected regions of interest in a subsequent frame. In some embodiments, classifying the specific tracked region of interest may comprise feeding an extracted portion of each processed frame of each track to the selected trained machine learning model. Each extracted portion of each processed frame may include the specific tracked region of interest. In some embodiments, classifying the specific tracked region of interest for each track may comprise classifying the specific tracked region of interest as one of at least the first content type and the second content type. In some embodiments, classifying the specific tracked region of interest may further comprise classifying the specific tracked region of interest as at least one content subtype. In some embodiments, the method may further comprise generating an annotated image. In some embodiments, generating the annotated image may comprise depicting a representation of the displacement path of each specific tracked region of interest on a selected frame from each of the at least one series of frames.

In accordance with another example embodiment of the present disclosure, an example particulate inspection system for detecting and classifying contents of interest within a medical reservoir may comprise a reservoir rest body having a pattern of light and dark regions. The system may further comprise a rest body backlight. The system may further comprise an external illuminator. The system may further comprise a vision assembly opposite the rest body. The vision assembly may include at least one imager. The system may further comprise a controller in data communication with each of the at least one imager. The controller may be configured to command capture of a series of source images from each of the at least one imager, receive the source images, process the source images into processed images, detect regions of interest within the processed images, and classify the detected regions of interest with a selected trained machine learning model of a plurality of trained machine learning models. The plurality of trained machine learning models may each be associated with a respective region of the rest body. The selected trained machine learning models may be the trained machine learning model associated with the respective region of the rest body each of the detected regions of interest was detected over.

In some embodiments, the system may further comprise a rotary actuator coupled to the rest body. In some embodiments, the rest body backlight may emit white light. In some embodiments, the pattern may be a checkered pattern. In some embodiments, the system may further comprise a reservoir retainer container coupled to reservoir rest body. The reservoir retainer including a set of IV bag port graspers actuatable between an open state and port retaining state. In some embodiments, the external illuminator may be an underlight. In some embodiments, the external illuminator may include a plurality of LEDs electrically coupled in series. In some embodiments, the external illuminator may include a plurality collimated light emitters with a narrow beam angle less than 20°. In some embodiments, the external illuminator may include a polarizer. In some embodiments, each of the at least one imager may include a polarizing filter on a lens thereof. In some embodiments, the vision assembly may include a plurality of imagers, each having a field of view inclusive of a portion of the rest body. Each field of view of each imager may overlap the field of view of at least one other imager of the vision system. In some embodiments, a view axis each of the at least one imager may be oriented at an angle 30-35° from a plane extending perpendicular to a height axis of the rest body. In some embodiments, the controller may be further configured to assign each region of interest in each of the processed images to one or more track. Each of the one or more track may define a displacement path of an associated region of interest throughout the processed images. In some embodiments, for each track, the controller may be configured to extract an extracted portion of every processed image in which the associated region of interest is detected, the extracted portion including the associated region of interest. Each of the plurality of trained machine learning models may be configured to classify regions of interest by analyzing the extracted portions of the processed images for each track. In some embodiments, each of the trained machine learning models may be a convolutional neural network. In some embodiments, the controller may be configured generate a reservoir acceptability determination. The acceptability determination may be indicated as a failure if any contents of interest are classified as something other than a bubble. In some embodiments, the medical reservoir may be an IV bag.

In accordance with an example embodiment of the present disclosure, an example method of packaging fluid into a medical bag may comprise positioning the medical bag in a first position in which a septum in a port of the bag is aligned with a fluid dispensing sharp. The method may further comprise irradiating at least an exposed surface of the septum with an irradiation assembly. The method may further comprise positioning the medical bag in a second position in which the fluid dispensing sharp has pierced through the septum and is in fluid communication with an empty compartment within an interior volume of medical bag. The method may further comprise dispensing a predetermined amount of diluent from the fluid dispensing sharp into the medical bag. The predetermined amount of diluent may be dispensed at a flow rate which ramps up to a first target flow rate until an initial amount of diluent has been dispensed and ramps down to a second target flow rate until a second amount of diluent has been dispensed. The second amount of diluent may be the predetermined amount less an amount of diluent which will flow through an outlet valve as the outlet valve transitions to a closed state.

In some embodiments, positioning the medical bag in the first position may comprise positioning the port at least partially into an airflow channel surrounding the fluid dispensing sharp. In some embodiments, the method may further comprise flowing air through the airflow channel. In some embodiments, the method may further comprise providing a laminar flow of clean air to the airflow channel. In some embodiments, the method may further comprise removing fluid dispensing sharp from the port and withdrawing a volume of fluid from the dispensing sharp. In some embodiments, irradiating at least the exposed surface of the septum may comprise illuminating the exposed surface of the septum with UV light. In some embodiments, irradiating at least the exposed surface of the septum may comprise powering an array of UVC LEDs. In some embodiments, irradiating at least the exposed surface of the septum may comprise powering at least one antimicrobial light emitter. In some embodiments, positioning the medical bag in the first position may comprise displacing the port within an irradiation transmissive barrier. In some embodiments, irradiating at least the exposed surface of the septum may comprise also irradiating at least a portion of the fluid dispensing sharp. In some embodiments, irradiating at least the exposed surface of the septum may comprise irradiating the exposed surface of the septum with at least a prescribed dosage of irradiation. In some embodiments, the concentrate may be a solid concentrate. In some embodiments, the solid concentrate may be a concentrate for a solution selected from a list consisting of a saline solution, a sugar solution, a saline and sugar solution, normal saline, half normal saline, D5W, peritoneal dialysate, hemodialysis dialysate, Ringer's solution, lactated Ringer's solution, Hartmann's Solution, hypotonic solution, isotonic solution, and hypertonic solution. In some embodiments, the solid concentrate may be selected from a list consisting of, powder, lyophilized medical agent, crystalline concentrate, salt concentrate, sodium chloride concentrate, sugar concentrate, dextrose concentrate. In some embodiments, positioning the bag in at least one of the first position and second position may comprise directing the port with at least one guide.

In accordance with another example embodiment of the present disclosure, a medical reservoir chain may comprise a lead medical reservoir. The chain may further comprise a terminal medical reservoir coupled to a spool. The chain may further comprise a plurality of intermediate medical reservoir coupled in series abreast of one another. A first of the plurality of intermediate medical reservoirs may be coupled to the lead medical reservoir and a second of the plurality of intermediate reservoirs. A last of the plurality of intermediate reservoirs may be coupled to a penultimate of the intermediate reservoirs and the terminal medical reservoir. Each of the medical reservoirs may define an interior volume containing at least one concentrate.

In some embodiments, each of the medical reservoirs may be a bag. In some embodiments, each of the at least one concentrate may be selected from a list consisting of a saline brine, a sodium chloride brine, a sugar solution, a saline and sugar solution, a peritoneal dialysate precursor solution, a hemodialysis precursor solution, a Ringer's solution concentrate, lactated Ringer's solution concentrate, and Hartmann's solution concentrate, a powder, a lyophilized agent, a crystalline solid, a salt in crystalline form, crystalline sodium chloride, crystalline sugar, a solid dialysate precursor, a saturated solution, an unsaturated solution, an at least partially liquid concentrate, and an at least partially solid concentrate. In some embodiments, the interior volume of each of the medical reservoirs may be divided into multiple internal compartments by at least one frangible partition. In some embodiments, the interior volume of each of the medical reservoirs may include at least one first compartment which is devoid of contents and at least one second compartment at least partially filled with a concentrate. In some embodiments, the chain of medical reservoirs may include a coupling region between each of the medical reservoirs. In some embodiments, the chain of medical reservoirs may include a weakened section between each of the medical reservoirs. In some embodiments, the chain of medical reservoirs may include at least one score line between each of the medical reservoirs. In some embodiments, the chain of medical reservoirs may include a series of perforations between each of the medical reservoirs. In some embodiments, the chain of medical reservoirs includes at least 50 medical reservoirs.

In accordance with another example embodiment of the present disclosure, an example infeed assembly for introducing an IV bag chain into a system for producing and packing fluids may comprise a bag displacement assembly including a first actuator coupled to a first displacement stage including a bag retention assembly. The infeed assembly may further comprise a second actuator coupled to a second displacement stage including a set of IV bag port retainers. The infeed assembly may further comprise a chain orientation imager having a field of view inclusive of at least a portion of a chain infeed pathway between an inlet region for the chain and the second displacement stage. The infeed assembly may further comprise a controller configured to orchestrate grasping of a lead IV bag of the chain and displacement of the lead IV bag into engagement with the IV bag port retainers via commands to the bag displacement assembly. The controller may be configured to receive an image from the imager, detect a set of IV bag ports in the image, determine a levelness characterization based on location data of each of the set of IV bag ports, and generate a leveling command to the second actuator when the levelness characterization is in breach of a predefined threshold.

In some embodiments, the infeed assembly further comprises a third actuator coupled to a third displacement stage. A chain splitting assembly may be coupled to the third displacement stage. In some embodiments, the controller may be further configured to orchestrate grasping of a second IV bag of the chain directly upstream of the lead IV bag via commands to the bag displacement assembly and may be configured to command the third actuator to drive the chain splitting assembly through a weakened region between the lead IV bag and the second IV bag via commands to the third actuator. In some embodiments, the controller may be further configured to orchestrate grasping of a second IV bag of the chain directly upstream of the lead IV bag via commands to the bag displacement assembly and may be configured to command the third actuator to drive the chain splitting assembly from a first position on a first side of the chain infeed pathway to a second position on an opposing side of the chain infeed pathway. In some embodiments, the controller may be configured to detect the set of IV bag ports with a template matching analysis based on template image of each port of the set of IV bag ports. In some embodiments, each of template image may be a masked template. In some embodiments, the template matching analysis may be selected from a list consisting of a linear spatial filtering based template analysis, a square difference template matching analysis, a normalized square difference template matching analysis, a cross-correlation template matching analysis, a normalized cross-correlation template matching analysis, a correlation coefficient template matching analysis, a normalized cross-correlation coefficient template matching analysis, and an eigenspace template matching analysis. In some embodiments, the controller may be configured to detect the set of IV bag ports with a trained machine learning model. In some embodiments, the trained machine learning model may be a convolutional neural network. In some embodiments, the controller may be configured to define coordinates for a bounding box around each of the set of IV bag ports in the image and the levelness characterization may be based at least in part on a corresponding test point within each bounding box. In some embodiments, the corresponding test point may be a center point of each bounding box. In some embodiments, the levelness characterization may be based at least in part on an X-coordinate difference and a Y-coordinate difference between corresponding test points in at least two bounding boxes.

In accordance with another example embodiment of the present disclosure an example infeed assembly for introducing an IV chain into a system for producing and packing fluids may comprise a bag displacement assembly including a first actuator coupled to a first displacement stage including a bag retention assembly. The infeed assembly may further comprise a second actuator coupled to a second displacement stage including a set of IV bag port retainers. The infeed assembly may further comprise a chain orientation imager having a field of view inclusive of at least a portion of a chain infeed pathway between an inlet region for the chain and the second displacement stage. The infeed assembly may further comprise a controller configured to orchestrate grasping of a lead IV bag of the chain and displacement of the lead IV bag into engagement with the IV bag port retainers via commands to the bag displacement assembly. The controller may be configured receive an image from the imager, detect a set of IV bag ports in the image, determine a positional characterization for the chain based on location data of each of the set of IV bag ports, generate a positional adjustment command to the second actuator when at least one aspect of the positional characterization is in breach of at least one first acceptability criteria, and generate an error notification when at least one aspect of the position characterization is in breach of at least one second acceptability criteria.

In some embodiments, the infeed assembly may further comprise a third actuator coupled to a third displacement stage. A chain splitting assembly may be coupled to the third displacement stage. In some embodiments, the controller may be further configured to orchestrate grasping of a second IV bag of the chain directly upstream of the lead IV bag via commands to the bag displacement assembly and may be configured to command the third actuator to drive the chain splitting assembly through a weakened region between the lead IV bag and the second IV bag via commands to the third actuator. In some embodiments, the controller may be further configured to orchestrate grasping of a second IV bag of the chain directly upstream of the lead IV bag via commands to the bag displacement assembly and may be configured to command the third actuator to drive the chain splitting assembly from a first position on a first side of the chain infeed pathway to a second position on an opposing side of the chain infeed pathway. In some embodiments, the controller may be configured to detect the set of IV bag ports with a template matching analysis based on template image of each port of the set of IV bag ports. In some embodiments, each of template image may be a masked template. In some embodiments, the template matching analysis may be selected from a list consisting of a linear spatial filtering based template analysis, a square difference template matching analysis, a normalized square difference template matching analysis, a cross-correlation template matching analysis, a normalized cross-correlation template matching analysis, a correlation coefficient template matching analysis, a normalized cross-correlation coefficient template matching analysis, and an eigenspace template matching analysis. In some embodiments, the controller may be configured to detect the set of IV bag ports with a trained machine learning model. In some embodiments, the trained machine learning model may be a convolutional neural network. In some embodiments, the positional characterization may include a height characterization aspect and a levelness characterization aspect. In some embodiments, the controller may be configured to define coordinates for a bounding box around each of the set of IV bag ports in the image and the positional characterization may be based at least in part on a corresponding test point within each bounding box. In some embodiments, the corresponding test point may be a center point of each bounding box. In some embodiments, the positional characterization may be based at least in part on an X-coordinate difference and a Y-coordinate difference between corresponding test points in at least two bounding boxes. In some embodiments, the at least one first acceptability criteria includes a range of acceptable tilt angles and the position characterization includes a levelness characterization aspect determined by calculating a tilt angle for the chain using the location data of each of the set of IV bag ports. In some embodiments, the at least one second acceptability criteria may include a range of acceptable height locations and the positional characterization includes a height characterization aspect determined using the location data of each of the set of IV bag ports.

In accordance with another example embodiment of the present disclosure, an example method of feeding a chain of IV bags into a system for producing and packaging fluid may comprise grasping, with a bag retention assembly, a lead IV bag of the chain. The method may further comprise powering at least one first actuator to displace the bag retention assembly to a displaceable stage and displace a set of ports of the lead IV bag into engagement with IV bag port retainers of the displaceable stage. The method may further comprise capturing image data of at least a portion of a chain infeed pathway between an inlet region for the chain and the displaceable stage with a chain orientation imager. The method may further comprise detecting a set of IV bag ports in the image data and storing image location data for each of the set of IV bag ports. The method may further comprise determining a positional characterization for the chain based on location data of each of the set of IV bag ports. The method may further comprise generating a positional adjustment command to a second actuator coupled to the displaceable stage when at least one aspect of the position characterization is in breach of at least one first acceptability criteria.

In some embodiments, the method may further comprise grasping, with the bag retention assembly, a second IV bag of the chain directly upstream of the lead IV bag and displacing a chain splitting assembly through a weakened region between the lead IV bag and the second IV bag. In some embodiments, the detecting the set of ports in the image data may comprise receiving the image data with a controller and analyzing the image data with a template matching analysis. In some embodiments, the template matching analysis may be selected from a list consisting of a linear spatial filtering based template analysis, a square difference template matching analysis, a normalized square difference template matching analysis, a cross-correlation template matching analysis, a normalized cross-correlation template matching analysis, a correlation coefficient template matching analysis, a normalized cross-correlation coefficient template matching analysis, and an eigenspace template matching analysis. In some embodiments, detecting the set of ports in the image data may comprise feeding at least a portion of the image data to a trained machine learning model. In some embodiments, the trained machine learning model may be a convolutional neural network. In some embodiments, determining the positional characterization may comprise determining a levelness characterization aspect for the chain based on the location data for each of the set of IV bag ports and determining a height characterization aspect for the chain based on the location data for each of the set of IV bag ports. In some embodiments, the method may further comprise defining a bounding box for each of the set of IV bag ports detected in the image data and storing the image location data for each of the set of IV bag ports comprises storing image coordinate information relating to the bounding box. In some embodiments, determining the positional characterization may comprise calculating an X-coordinate difference and a Y-coordinate difference between corresponding test points in at least two bounding boxes. In some embodiments, the corresponding test points may be center points of each bounding box. In some embodiments, the at least one first acceptability criteria may include an acceptable chain levelness range. In some embodiments, the method may further comprise generating an error notification when at least one aspect of the positional characterization is in breach of at least one second acceptability criteria. In some embodiments, the at least one second acceptability criteria may include an acceptable chain height range.

In accordance with another example embodiment of the present disclosure an example valve assembly for adjusting airflow between compartments of an environmentally controlled enclosure may comprise a stator. The stator may include an elongate body with a round cross-section having a first open end, a second closed end, and an intermediate span. There may be a stator bore extending from the first open end toward the second closed end and a fenestration through the intermediate span to the stator bore. The fenestration may have a length dimension equivalent to at least 50% of an axial dimension of the stator. The fenestration may be present over a fenestration arc of the elongate body having a degree measure of at least 100°. The valve assembly may further comprise a rotor. The rotor may have cradle portion partially enveloping but spaced from the stator by a gap distance. The cradle portion may project parallel to the axial dimension of the stator and may be arced about the axial dimension of the elongate body over a cradle arc having a degree measure greater than that of the fenestration arc but not more than 360° minus the degree measure of the fenestration arc. The valve assembly may further comprise a rotary actuator coupled to the rotor.

In some embodiments, the rotary actuator may include rotary position sensor. In some embodiments, the stator may include a mounting flange at the open end. In some embodiments, a sidewall of the fenestration may be rounded. In some embodiments, the cradle portion may include an exterior most edge which is rounded. In some embodiments, the rotor may be displaceable via the rotary actuator to drive the cradle portion between a fenestration covering position, a plurality of positions in which the fenestration is partially covered by the cradle portion, and a position in which the fenestration is uncovered by the cradle portion. In some embodiments, in the fenestration covering position, the fenestration may be covered by a central region of the cradle portion and a portion of the elongate body adjacent each opposing side of the fenestration may be covered by respective regions of the cradle portion which flank the central region of the cradle portion. In some embodiments, the gap distance may provide a fluid communication pathway from the stator bore to an exterior of the valve assembly when the cradle portion is in the fenestration covering position.

In accordance with another example embodiment of the present disclosure, an example fluid handling system for producing medical fluid may comprise a source input. The system may further comprise an accumulator reservoir including an accumulator fluid heater. The accumulator reservoir may have a first input flow path including a heat exchanger and a second input flow path which bypasses the heat exchanger. The first and second input flow path may be selectively coupled to the source input via an input flow path selection valve. The system may further comprise at least one reverse osmosis filtration assembly coupled to an output of the accumulator reservoir. The system may further comprise at least one ultrafilter in fluid communication with an output of the at least one reverse osmosis assembly. The system may further comprise a dispensing cartridge in fluid communication with an output of the ultrafilter. The system may further comprise a plurality of fluid characteristic sensors including a plurality of temperature sensors. The system may further comprise at least one heat disinfection heater. The system may further comprise a controller that controls the flow of fluid through the fluid handling system. The controller may be configured to orchestrate a disinfection of a portion of the fluid handling system downstream of the accumulator reservoir by adjusting respective duty cycle commands to each of the accumulator fluid heater and the at least one heat disinfection heater based on data signal from the plurality of temperature sensors and a disinfection temperature set point and by actuating the input flow path selection valve to couple the source input to the second input flow path.

In some embodiments, the system may further comprise an electrodeionization unit intermediate the at least one reverse osmosis filtration assembly and the ultrafilter. In some embodiments, the system may further comprise a first UV light emitting assembly intermediate the accumulator and the at least one reverse osmosis filtration assembly and a second UV light emitting assembly intermediate the at least one reverse osmosis filtration assembly and the at least one ultrafilter. In some embodiments, the plurality of fluid characteristic sensors may include a total organic carbon sensor assembly on a slip stream extending from a fluid communication conduit between the at least one ultrafilter and the dispensing cartridge. In some embodiments, the system may further comprise at least one carbon filter intermediate the source input and the accumulator reservoir. In some embodiments, the plurality of fluid characteristic sensors may include a permeate conductivity sensor in communication with a permeate flow path from the at least one reverse osmosis filtration assembly and a plurality of conductivity sensors in communication with a flow path extending from the at least one ultrafilter to the dispensing cartridge. In some embodiments, the controller may be further configured to orchestrate a fluid handling system disinfection cool down by reducing the respective duty cycle commands to each of at least the at least one heat disinfection heater to zero percent at a predefined ramp rate. In some embodiments, the at least one reverse osmosis filtration assembly may include a retentate recirculation pathway. There may be a retentate drain flow path coupled to the retentate recirculation flow path via retentate drain control valve. The plurality of fluid characteristic sensors may include a retentate drain flow rate sensor. The controller may be configured to command actuation of the retentate drain control valve based on data from the retentate drain flow rate sensor and a disinfection flow rate target when orchestrating disinfection of the portion of the fluid handling system. In some embodiments, the controller may be further configured to orchestrate a fluid handling system disinfection cool down by reducing the respective duty cycle commands to each of at least the at least one heat disinfection heater to zero percent at a predefined ramp rate and by commanding actuation of the retentate drain control valve, based on data from the retentate drain flow rate sensor, to increase a flow rate of fluid to the retentate flow path drain to a cool down flow rate target at least triple the disinfect flow rate target.

In accordance with yet another example embodiment of the present disclosure, a system for packaging fluid into a medical reservoir may comprise a medical fluid production assembly. The system may further comprise a dispenser containing a chain of reservoirs. The system may further comprise an enclosure having a plurality of compartments and an infeed aperture. The system may further comprise an air handling assembly including an intake, a blower, at least one filter, and an output in communication with the enclosure. The system may further comprise a dispenser interface assembly including a dispenser outlet cover retainer assembly displaceable between a stowed position and an infeed aperture plugging position. The system may further comprise a reservoir singulation assembly. The system may further comprise filling assembly including a cartridge having an inlet in fluid communication with the medical fluid production assembly and outlet including a dispensing sharp. The system may further comprise a particulate inspection assembly. The system may further comprise a marking assembly. The system may further comprise an outfeed assembly including a plurality of reservoir receptacles. The system may further comprise a database arrangement. The system may further comprise a controller in data communication with and receiving process data from the medical fluid production assembly, air handling assembly, dispenser interface assembly, reservoir interface assembly, filling assembly, particulate inspection system, marking assembly. The controller may be configured to collect and analyze the process data into analyzed data, serialize at least a portion of the process data and at least a portion of the analyzed data into at least one reservoir processing record and communicate each of the at least one reservoir processing record to the database arrangement.

In some embodiments, the database arrangement may include a record aggregator and a database. In some embodiments, the fluid handling system may include a plurality of fluid characteristic sensors and the analyzed data may include statistical information based on the output signals of the plurality of fluid characteristic sensors. In some embodiments, the air handling system may include a plurality of air characteristic sensors and the analyzed data may include statistical information based on the output signals of the plurality of air characteristic sensors. In some embodiments, the dispenser may include a dispenser indicia and the dispenser interface assembly may include a dispenser indicia reader. The controller may be configured to receive indicia data from the reader and determine a unique identifier for the respective dispenser from the indicia data. In some embodiments, the controller may be further configured to generate and communicate an inventory query to the database arrangement including the unique identifier and the database arrangement may be configured to return a result set including at least a usage acceptability indication. In some embodiments, the cartridge may include a cartridge indicia and the filling assembly may include a cartridge indicia reader. The controller may be configured to receive indicia data from the reader and determine a unique identifier for the respective cartridge from the indicia data. In some embodiments, the controller may be further configured to generate and communicate an inventory query to the database arrangement including the unique identifier and the database arrangement may be configured to return a result set including at least a usage acceptability indication. In some embodiments, the particulate inspection assembly may include a mixing assembly. In some embodiments, the particulate inspection assembly may include at least one imager. The process data may include one or more raw frames from the imager and the analyzed data may include reservoir content of interest data determined from the one or more raw frames. In some embodiments, the system may further comprise a marking verification imager. The controller may be configured to receive image data from the marking verification imager and determine a marking content of a marking from the marking assembly on a reservoir from the image data. In some embodiments, the analyzed data may include the marking content determined from the image data from the marking verification imager. In some embodiments, the outfeed assembly may further comprise a plurality of locks and the system may further comprise at least one user interface. In some embodiments, the user interface may be configured to communicate an access request input including a user credential identifier and the controller may be configured to generate one or more actuation command for the plurality of locks based one the user credential identifier.

In accordance with another example embodiment of the present disclosure, a dispenser of medical reservoirs may comprise a housing having an outlet. The dispenser may further comprise a cover assembly sealing the outlet and may be coupled to the housing via a lock assembly. The dispenser may further comprise a reel rotatably coupled within the housing. The dispenser may further comprise a chain of abreastly coupled medical bags coupled to the reel via a tail portion of the chain. The dispenser may further comprise a tensioner assembly coupled to the reel. The dispenser may further comprise a bag holder with a set of port retainers coupled thereto. A set of ports of a lead bag of the chain of medical bags may be retained in the set of port retainers.

In some embodiments, the dispenser may further comprise at least one handle. The handle may be coupled to the housing on a side of the housing opposite the outlet. In some embodiments, the housing may include at least one handle disposed on a side of the dispenser opposite the outlet. In some embodiments, the bag holder may be formed integrally with a portion of the housing. In some embodiments, a first side of the housing may include a first and second face. The first face may be substantially parallel to an opposing side of the housing. The second face may extend from an edge of the first face at an angle toward the opposing side of the housing. In some embodiments, the outlet may be disposed in the second face. In some embodiments, the cover assembly may include a backing plate with a sealing member, an overlay body, and a pair of lock bodies of the lock assembly captured between the backing plate and overlay body. In some embodiments, the overlay body may include a number of slits therethrough. Each slit may align with an aperture extending through one of the pair of lock bodies. In some embodiments, each slit may align with a respective variable depth trough in the backing body. Each variable depth trough may be formed partially by a pocket which presents a stepwise change in the depth of the variable depth trough. In some embodiments, the cover assembly may include a set of guides, each lock body including a guide slot which accepts a respective guide of the set of guides. The lock bodies may be translationally displaceable between a retracted state in which the lock bodies are disposed adjacent one another to a deployed state in which the lock bodies are spread apart from one another. In some embodiments, each set of guides may include at least one roller. In some embodiments, each of the lock bodies may include a set of bolt projections. A terminal end of each bolt projection may have a ramped surface. In some embodiments, the dispenser may further comprise a rim surrounding the outlet. The rim may include a plurality of bolt retainers of the lock assembly and sealing member directly adjacent the outlet. In some embodiments, the port retainers may be displaceable relative to the bag holder. In some embodiments, the housing may include a flange. The flange may include a set of retaining apertures. In some embodiments, each of the medical bags may contain at least one concentrate selected from a list consisting of a saline brine, a sodium chloride brine, a sugar solution, a saline and sugar solution, a peritoneal dialysate precursor solution, a hemodialysis precursor solution, a Ringer's solution concentrate, lactated Ringer's solution concentrate, and Hartmann's solution concentrate, a powder, a lyophilized agent, a crystalline solid, a salt in crystalline form, crystalline sodium chloride, crystalline sugar, a solid dialysate precursor, a saturated solution, an unsaturated solution, an at least partially liquid concentrate, and an at least partially solid concentrate. In some embodiments, each of the medical bags may include an interior volume divided into multiple internal compartments by at least one frangible partition. In some embodiments, each of the medical bags may include an interior volume with at least one first compartment which is devoid of contents and at least one second compartment at least partially filled with a concentrate. In some embodiments, the chain may include a weakened section between each of the medical bags. In some embodiments, the chain may include at least one score line between each of the medical bags. In some embodiments, the chain may include a series of perforations between each of the medical bags. In some embodiments, the chain may comprise at least 50 medical bags. In some embodiments, the tail may include at least one medical bag.

In accordance with another example embodiment of the present disclosure a method of packaging medical agent into medical reservoirs may comprise feeding a chain of reservoirs from a dispenser into a processing compartment including a filling station. The method may further comprise segregating individual reservoirs from the chain. The method may further comprise filling the individual reservoirs at the filling station. The method may further comprise segregating a last consumable reservoir from a tail of the chain. The method may further comprise retracting the tail into the dispenser.

In some embodiments, the method may further comprise docking the dispenser to the processing compartment and unsealing the dispenser. In some embodiments, unsealing the dispenser may comprise positively engaging a removably cover assembly of the dispenser with a sanitary interface assembly and displacing at least a portion of the sanitary interface assembly with which the removable cover assembly is positively engaged into a stowed location within the processing compartment. In some embodiments, feeding the chain of reservoirs into the processing compartment may comprise unspooling the chain of reservoirs from a reel disposed within the dispenser. In some embodiments, the feeding the chain of reservoirs into the processing chamber may comprise tensioning a tensioner assembly of the dispenser and retracting the tail into the dispenser may comprise driving retraction of the tail with the tensioning assembly. In some embodiments, segregating individual reservoirs from the chain may comprise driving a splitter assembly of a reservoir individualizing assembly through a weakened region between a to-be-separated reservoir and a next reservoir on the chain. In some embodiments, segregating individual reservoirs from the chain may comprise tearing the individual reservoirs from the chain along weakened regions between reservoirs of the chain. In some embodiments, filling the individual reservoirs may comprise dispensing a diluent into the individual reservoirs. In some embodiments, filling the individual reservoirs may comprise puncturing a septum of each of the individual reservoirs with a dispensing sharp and dispensing a mass of excipient into each of the individual reservoirs based on a capacity of the respective reservoir and a concentrate in the respective reservoir. In some embodiments, the method may further comprise irradiating a septum of each of the individual reservoirs. In some embodiments, the medical reservoirs may be bags and the bags may be abreastly coupled together to form the chain.

In accordance with another example embodiment of the present disclosure an outfeed assembly for a system for producing and packaging fluids may comprise a compartment. The outfeed assembly may further comprise a drawer including a first receptacle array of a first number of first receptacles and a second receptacle array of a second number of second receptacles less than the first number. The outfeed assembly may further comprise a drawer slide assembly including a carriage coupled to the drawer and including a carriage striker. The drawer slide assembly may further comprise a telescoping assembly including a telescoping assembly striker. The drawer slide assembly may further comprise a base fixed relative to the compartment including a carriage lock, a telescoping assembly lock, and a set of drawer slides coupled to the carriage. The drawer may be locked in a closed position when the carriage striker and telescoping assembly striker are respectively engaged with the carriage lock and telescoping assembly lock. The outfeed assembly may further comprise a user interface. The outfeed assembly may further comprise a controller. The controller may be configured to determine a credential associated with a user identity input to the user interface. The controller may be further configured to disengage the carriage lock upon receipt of a first input to the user interface by a user associated with either of a first credential and second credential. The controller may be further configured to disengage the telescoping lock upon receipt of a second input to the user interface by a user associated with the second credential. The drawer may be within the compartment in the closed position. The drawer may be displaceable from the closed position to a first open position when the carriage lock is disengaged and displaceable to a second open position when the carriage and telescoping assembly locks are disengaged. The second receptacle array may be within the compartment at the first open position and out of the compartment in the second open position.

In some embodiments, the outfeed assembly may further comprise a warding grid associated with the outfeed drawer. The warding grid may be disposed intermediate the outfeed drawer and the remainder of the outfeed compartment. The warding grid may include a number of apertures which align with the first and second receptacles of the outfeed drawer when the associated outfeed drawer is in the closed state. In some embodiments, the second receptacle array may be one of raised, recessed, and at even height with respect to the first receptacle array. In some embodiments, a receptacle state detector may be included in each of the first receptacles and each of the second receptacles. In some embodiments, each receptacle state detector may output a first signal when the respective receptacle is empty and a second signal when the respective receptacle contains a reservoir. In some embodiments, the outfeed assembly may further comprise a gantry assembly having a medical reservoir grasper with a pair of jaws actuatable between a closed state and an open state. In some embodiments, the gantry assembly may include an imager. In some embodiments, the first receptacles may be arranged in at least one first receptacle row and the second receptacles may be arranged in at least one second receptacle row and the outfeed assembly further may comprise a plurality of time of flight outfeed inventory sensors. Each first receptacle row and each second receptacle row may be associated with a respective time of flight inventory sensor of the plurality of time of flight outfeed inventory sensors. In some embodiments, each receptacle in each first receptacle row may include a pair first receptacle apertures aligned with a sensing beam axis of the time of flight inventory sensor associated with the first receptacle row. Each receptacle in each second receptacle row may include a pair of second receptacle apertures aligned with the sensing beam axis of the time of flight inventory sensor associated with the first receptacle row. In some embodiments, the outfeed assembly may further comprise a plurality of outfeed inventory imagers. In some embodiments, the drawer slide assembly may include at least one set of carriage displacement dampers. In some embodiments, the drawer slide assembly may include a first set of carriage displacement dampers positioned to engage the carriage when the carriage is displaced to the closed position. The drawer slide assembly may include a second set of displacement dampers positioned to engage the carriage when the carriage is displaced to the first open position. In some embodiments, the drawer slide assembly may include at least one set of telescoping assembly displacement dampers positioned to engage the telescoping assembly when the telescoping assembly is displaced to an end of a displacement range of the telescoping assembly. In some embodiments, the telescoping assembly may be coupled to guides on the base. In some embodiments, the base may include a bumper. The telescoping assembly may collide with the bumper when the drawer is displaced to the second open position. The bumper may prohibit further displacement of the drawer in a direction away from the compartment. In some embodiments, at least one drawer position sensor may be coupled to the base. The controller may be in data communication with each of the at least one drawer position sensor. In some embodiments, the telescoping assembly may include a set of stops. The carriage may including a set of projections. The projections may collide with the stops when the drawer is displaced to the first open position.

In accordance with another example embodiment of the present disclosure, a method of packing fluid into a medical reservoir may comprise loading a dispenser against an enclosure. The method may further comprise removing and stowing an outlet cover of the dispenser within a first compartment of the enclosure. The method may further comprise drawing a chain of reservoirs into the first compartment from the dispenser. The method may further comprise singulating an individual reservoir from the chain. The method may further comprise filling the individual reservoir with fluid from a medical fluid production assembly into the individual reservoir through a dispensing sharp which has pierced a septum of the individual reservoir. The method may further comprise displacing and isolating the individual reservoir into a second compartment of the enclosure. The method may further comprise displacing the individual reservoir into a third compartment of the enclosure. The method may further comprise inspecting the individual reservoir. The method may further comprise marking the individual reservoir with a marking dependent on the outcome of the inspection. The method may further comprise displacing the individual reservoir to a receptacle. The receptacle may depend on the outcome of the inspection.

In some embodiments, the method may further comprise environmentally controlling a plurality of zones within the enclosure. The zones may be in at least one compartment of an enclosure. In some embodiments, a first of the plurality of zones may be directly adjacent the dispensing sharp and a second of the plurality of zones may be the remainder of the first compartment. The first zone may be more stringently controlled than the second. In some embodiments, the second compartment may be one of the plurality of zones and the second compartment may be controlled to a level of control at least equal to a level of control of a zone in the first compartment of the enclosure before the individual reservoir is displaced into the second compartment. In some embodiments, removing and stowing the outlet cover may comprise forming a seal between the outlet cover and exposed portions of an interface assembly and retaining the outlet cover on the interface assembly. In some embodiments, retaining the outlet cover on the interface assembly may comprise drawing a vacuum in a volume present between the exposed portion of the interface assembly and the outlet cover. In some embodiments, retaining the outlet cover on the interface assembly may comprise positively engaging the outlet cover with the interface assembly. In some embodiments, positively engaging the outlet cover with the interface assembly may comprise capturing a portion of the outlet cover in notches defined in projections extending from the interface assembly In some embodiments, drawing the chain into the first compartment may comprise grasping a lead reservoir of the chain and dislodging it from a retainer on a reservoir presenter of the dispenser. In some embodiments, singulating the individual reservoir from the chain may comprise tearing the individual reservoir from the chain along a weakened region of the chain. In some embodiments, singulating the individual reservoir from the chain may comprise driving a splitter of a bag individualizing assembly through a weakened region of the chain. In some embodiments, filling the individual reservoir may comprise irradiating at least an exposed portion of a septum of the individual reservoir. In some embodiments, irradiating at least the exposed portion of the septum may comprise irradiating a portion of the dispensing sharp. In some embodiments, inspecting the individual reservoir may comprise agitating the individual reservoir. In some embodiments, agitating the individual reservoir may comprise rotating the individual reservoir between pairs of rotational orientations in accordance with a predefined motion profile. In some embodiments, inspecting the individual reservoir may comprise providing a contrasting pattern on a first side of the individual reservoir and illuminating the individual reservoir with collimated, polarized light from an external illuminator. In some embodiments, inspecting the reservoir may further comprise capturing a plurality of images with an imager associated with a polarizing filter, processing the images, detecting pixel clusters of interest within the images and determining classifications for the pixel clusters of interest. In some embodiments, the classifications may be selected from a list consisting of, a first content type, a second content type, a content subtype, a content identity, gas, bubble, not a bubble, particulate, a particulate type, and a content size. In some embodiments, the marking may indicate the individual reservoir is not to be used when at least one pixel cluster of interest is assigned a class predetermined to be unacceptable. In some embodiments, when at least one pixel cluster of interest is assigned a class predetermined to be unacceptable, the receptacle may be an access controlled receptacle. In some embodiments, marking the individual reservoir may comprise embossing the marking into a portion of the individual reservoir. In some embodiments, the receptacle may be included in an outfeed drawer. In some embodiments, the method may further comprise verifying the marking matches an intended marking. The receptacle may be dependent upon the inspection and the verification. In some embodiments, filling the individual reservoir may comprise providing diluent to a concentrate disposed in the individual reservoir. In some embodiments, the method may further comprise verifying the individual reservoir was deposited into the receptacle. In some embodiments, verifying the individual reservoir was deposited into the receptacle may comprise comparing an image of the receptacle to a reference image of the receptacle. In some embodiments, verifying the individual reservoir was deposited into the receptacle may comprise monitoring an inventory sensor associated with the receptacle for an output signal indicative that the individual reservoir was deposited into the receptacle.

In accordance with another example embodiment of the present disclosure a system for packaging fluid into a medical reservoir may comprise a medical fluid production assembly. The system may further comprise a dispenser containing a chain of reservoirs. The system may further comprise an enclosure having a plurality of compartments. A first of the compartments may include an infeed aperture. The system may further comprise a dispenser interface assembly including a dispenser outlet cover retainer assembly displaceable between a stowed position and an infeed aperture plugging position. The system may further comprise a reservoir singulation assembly in the first compartment. The system may further comprise a filling assembly including cartridge having an inlet in fluid communication with the medical fluid production assembly and outlet including a dispensing sharp. The cartridge may be at least partially in the first compartment. The system may further comprise a second compartment. There may be a first door between the first and second compartments. The system may further comprise a third compartment. There may be being a second door between the second and third compartments. The system may further comprise a particulate inspection system in the third compartment. The system may further comprise a marking assembly in the third compartment. The system may further comprise an outfeed assembly in the third compartment including at least one first array of reservoir receptacles and at least one second array of reservoir receptacles.

In some embodiments, the medical fluid production assembly may include a plurality of water treatment apparatuses selected from a group consisting of a carbon filter, a reverse osmosis filter, an electrodeionization assembly, and a UV light emitter. In some embodiments, the chain of reservoirs may be a chain of abreastly chained IV bags. In some embodiments, each reservoir in the chain of reservoirs may include at least one concentrate. In some embodiments, each of the at least one concentrate may be selected from a list consisting of a saline brine, a sodium chloride brine, a sugar solution, a saline and sugar solution, a peritoneal dialysate precursor solution, a hemodialysis precursor solution, a Ringer's solution concentrate, lactated Ringer's solution concentrate, and Hartmann's solution concentrate, a powder, a lyophilized agent, a crystalline solid, a salt in crystalline form, crystalline sodium chloride, crystalline sugar, a solid dialysate precursor, a concentrate for a hypotonic solution, a concentrate for an isotonic solution, a concentrate for a hypertonic solution, a saturated solution, an unsaturated solution, an at least partially liquid concentrate, and an at least partially solid concentrate. In some embodiments, the system may further comprise a reservoir displacement assembly in the first compartment. The reservoir displacement assembly may include a least one robotic gripper assembly coupled to a linear displacement stage. In some embodiments, the dispenser may include a dispenser outlet, a dispenser outlet cover, and a dispenser lock assembly. Thee outlet cover may be in sealing relationship with the dispenser about the dispenser outlet when the lock assembly is in a locked state. In some embodiments, the dispenser outlet cover interface may include a dispenser lock assembly actuation assembly. In some embodiments, the dispenser lock assembly actuation assembly may include a set of projections sized to fit in respective apertures of the dispenser lock assembly. In some embodiments, each of the projections may include a notch sized to accept a portion of the dispenser outlet cover. In some embodiments, the dispenser outlet cover interface may include a pressure port in selective fluid communication with a negative pressure source. In some embodiments, the reservoir singulation assembly may include a splitter assembly coupled to a splitter assembly actuator. The splitter assembly displaceable through a range from a raised position to an end of stroke position via the splitter assembly actuator. In some embodiments, the filling assembly may further comprise an irradiation assembly including a plurality of antimicrobial light emitter and the cartridge includes a barrier surrounding at least a portion of the dispensing sharp which is at least partially transparent to the antimicrobial light. In some embodiments, the particulate inspection assembly may include a reservoir rest coupled to a rotary actuator. In some embodiments, the system may further comprise a controller in data communication with the rotary actuator. The controller may be configured to command displacement of the rotary actuator between preset pairs or rotational orientations defined in a motion profile. In some embodiments, the particulate inspection system may include a contrasting pattern, an illuminator including at least one collimated light emitter and a polarizer, and at least one imager having a polarizing filter disposed opposite the contrasting pattern. In some embodiments, the system may further comprise a controller configured to receive image data from the at least one imager, process the image data, detect pixel clusters of interest in the image data, and determine classifications for the pixel clusters of interest. In some embodiments, the classifications may be selected from a list consisting of, a first content type, a second content type, a content subtype, a content identity, gas, bubble, not a bubble, particulate, a particulate type, and a content size. In some embodiments, the controller may be configured to supply a portion of the image data including at least the pixel clusters of interest to a convolutional neural network trained on images of reservoirs including known contents to determine the classifications. In some embodiments, the marking assembly may include an embossing head coupled to an embossing actuator. In some embodiments, one of the at least one a first array of reservoir receptacles and one of the at least one a second array of reservoir receptacles may be included in an outfeed drawer. In some embodiments, the outfeed assembly may include a first lock for the outfeed drawer and a second lock for the outfeed drawer. The outfeed drawer may be displaceable to a partially open position when the first lock is disengaged and a fully opened position when the second lock is disengaged. In some embodiments, each receptacle of the first array of reservoir receptacles may include a respective first receptacle reservoir presence sensor and each receptacle of the second array of reservoir receptacles may include a second array of reservoir presence sensors. In some embodiments, the first array of reservoir receptacles may be arranged in at least one first array row and the second array of reservoir receptacles may be arranged in at least one second array row. Each of the receptacles in the first array and second array of reservoir receptacles may include a pair of aligned apertures. Each of the at least one first array row and each of the at least one second array row may be associated with a time of flight outfeed inventory sensor. In some embodiments, the outfeed assembly may include a plurality of outfeed inventory imagers and the system may further comprise a control system. The control system may be configured to compare images from the outfeed inventory imager to respective reference images for each of the outfeed inventory imagers and, based on the comparisons, determine which receptacles of the first array of reservoir receptacles and at least one second array of reservoir receptacles contain reservoirs.

In accordance with another example embodiment of the present disclosure a method of verifying hardware functionality of a particulate inspection system for inspecting reservoirs may comprise capturing a first test image of a portion of the particulate inspection system in a first condition. The method may further comprise performing a first comparison, with a controller, of the first test image with a first reference image and generating a first fault notification with the controller when the first comparison fails a first quality criteria. The method may further comprise capturing a second test image of the portion of the particulate inspection system in a second condition when the first comparison passes the first quality criteria. The method may further comprise performing a second comparison, with the controller, of the second test image with a second reference image and generating a second fault notification with the controller when the second comparison fails a second quality criteria. The method may further comprise capturing a third test image of the portion of the particulate inspection system in a third condition when the second comparison passes the second quality criteria. The method may further comprise performing a third comparison, with the controller, of the third test image with a third reference image and generating a third fault notification with the controller when the third comparison fails a third quality criteria. The method may further comprise populating an inspection log for the reservoir with results of the first, second, and third comparison.

In some embodiments, the first condition may be a condition in which a backlight and external illuminator of the particulate inspection system are unpowered. In some embodiments, the second condition may be a condition in which a backlight of the particulate inspection system is powered and the portion of the particulate inspection system may be a patterned rest body which is illuminated by the back light. In some embodiments, the third condition may be a condition in which an external illuminator of the particulate inspection system is powered. In some embodiments, performing the first comparison may comprise aligning the first test image with the first reference image. In some embodiments, performing the first comparison may comprise performing a mean square error analysis with the first test image and first reference image. In some embodiments, the first quality criteria may be a mean square error threshold above which the first comparison is failed. In some embodiments, performing the second comparison may comprise aligning the second test image with the second reference image. In some embodiments, performing the second comparison may comprise performing a mean square error analysis with the second test image and second reference image. In some embodiments, the second quality criteria may be a mean square error threshold above which the second comparison is failed. In some embodiments, performing the third comparison may comprise aligning the third test image with the third reference image. In some embodiments, performing the third comparison may comprise performing a mean square error analysis with the third test image and third reference image. In some embodiments, the third quality criteria may be a mean square error threshold above which the third comparison is failed. In some embodiments, the method may further comprise installing the reservoir in the particulate inspection system and capturing a fourth test image of the portion of the particulate inspection system. In some embodiments, the method may further comprise performing a fourth comparison of the fourth test image to a fourth reference image and generating a notification, with a controller, when the fourth comparison fails a fourth quality criteria. In some embodiments, the fourth reference image may be of the portion of the particulate inspection system with no reservoir installed in the particulate inspection system. In some embodiments, performing the fourth comparison may comprise aligning the fourth test image against the fourth reference image and performing a mean square error analysis. The fourth quality criteria may be a mean square error threshold below which the fourth quality criteria is failed.

In accordance with another example embodiment of the present disclosure, an example fluid dispensing assembly for filling reservoirs with medical fluid may comprise a cartridge. The cartridge may comprise an irradiation transmissive barrier. The cartridge may further comprise an airflow channel. The irradiation transmissive barrier may define at least a portion of the airflow channel. The cartridge may further comprise a dispensing sharp coupled to a hub within the airflow channel. The cartridge may further comprise a connection port having a plurality of sealing members in spaced relation about an exterior surface of the connection port. The cartridge may further comprise a frangible seal at a terminal end of the connection port. The cartridge may further comprise a flow path extending from the connection port to the dispensing sharp and having at least one filter disposed in an intermediate portion of the flow path. The fluid dispensing assembly may further comprise an irradiation assembly including at least one antimicrobial light emitter. The at least one antimicrobial light emitter may be positioned about the irradiation transmissive barrier.

In some embodiments, the assembly may further comprise a cartridge dock. In some embodiments, the assembly may further comprise a supply manifold having a receptacle sized to accept the connection port. The receptacle may include a spike. In some embodiments, the irradiation assembly may include a set of UVC LEDs. In some embodiments, the at least one antimicrobial light emitter may include at least 3 antimicrobial light emitters spaced about the irradiation transmissive barrier. Each antimicrobial light emitter may be positioned 90° from at least one other antimicrobial light emitter. In some embodiments, the dispensing sharp may be a pencil tipped sharp with two side ports disposed in opposition to one another. In some embodiments, the irradiation transmissive barrier may be at least partially transparent to UVC light. In some embodiments, the irradiation transmissive barrier may be formed of fused quartz. In some embodiments, the airflow channel may extend along a straight line path through the cartridge and may have a round cross-sectional shape. In some embodiments, at least one guide may be included at a first end of the airflow channel. In some embodiments, the cross-sectional area of the airflow channel may varies over the length of the airflow channel.

In accordance with another example embodiment of the present disclosure, a fluid dispensing assembly for filling reservoirs with medical fluid may comprise a cartridge. The cartridge may comprise an irradiation transmissive barrier. The cartridge may further comprise an airflow channel. The irradiation transmissive barrier may define at least a portion of the airflow channel. The cartridge may further comprise a dispensing sharp coupled to a hub within the airflow channel. The cartridge may further comprise a connection port having a plurality of sealing members in spaced relation about an exterior surface of the connection port. The cartridge may further comprise a frangible seal at a terminal end of the connection port. The cartridge may further comprise a flow path extending from the connection port to the dispensing sharp and having at least one filter disposed in an intermediate portion of the flow path. The fluid dispensing assembly may further comprise an irradiation assembly including at least one antimicrobial light emitter. The at least one antimicrobial light emitter may be positioned about the irradiation transmissive barrier. The dispensing assembly may further comprise an antimicrobial agent dispenser.

In some embodiments, the assembly may further comprise a cartridge dock. In some embodiments, the assembly may further comprise a supply manifold having a receptacle sized to accept the connection port. The receptacle may including a spike. In some embodiments, the irradiation assembly may include a set of UVC LEDs. In some embodiments, the at least one antimicrobial light emitter may include at least three antimicrobial light emitters spaced about the irradiation transmissive barrier. Each antimicrobial light emitter may be positioned 90° from at least one other antimicrobial light emitter. In some embodiments, the dispensing sharp may be a pencil tipped sharp with two side ports disposed in opposition to one another. In some embodiments, the irradiation transmissive barrier may be at least partially transparent to UVC light. In some embodiments, the irradiation transmissive barrier may be formed of fused quartz. In some embodiments, the airflow channel may extend along a straight line path through the cartridge and has a round cross-sectional shape. In some embodiments, at least one guide may be included at a first end of the airflow channel. In some embodiments, the cross-sectional area of the airflow channel may vary over the length of the airflow channel. In some embodiments, the antimicrobial agent dispenser may include one of a sprayer and an atomizer with an outlet directed to a point below an end of the dispensing sharp. In some embodiments, the antimicrobial agent dispenser may include a removable agent source reservoir. In some embodiments, the antimicrobial agent dispenser may be an isopropyl alcohol dispenser.

In accordance with another example embodiment of the present disclosure an example method of packaging fluid into a medical bag may comprise positioning the medical bag in a first position in which a septum in a port of the bag is aligned with a fluid dispensing sharp. The method may further comprise dispensing an antimicrobial agent onto at least an exposed portion of the septum. The method may further comprise illuminating at least the exposed portion of the septum with at least one antimicrobial light emitter. The method may further comprise positioning the medical bag in a second position in which the fluid dispensing sharp has pierced through the septum and is in fluid communication with an empty compartment within an interior volume of medical bag. The method may further comprise dispensing a predetermined amount of fluid from the fluid dispensing sharp into the medical bag. The method may further comprise retaining the medical bag in a mix assisting assembly and rotating the medical bag in alternating clockwise and counterclockwise directions about an axis substantially perpendicular to a plane of the medical bag in which the axis of the port falls through a sequence of predetermined positions.

In some embodiments, the medical bag may be rotated at at least a certain rotation rate as the medical bag is rotated through the sequence of predetermined positions. In some embodiments, the method further may comprise dwelling at each of the predetermined positions. In some embodiments, rotating the medical bag comprises fully inverting the medical bag at least once. In some embodiments, retaining the medical bag in the mix assisting assembly may comprise placing the medical bag in a cradle of the mix assisting assembly. In some embodiments, the method may further comprise mixing the fluid with a concentrate in the medical bag by compromising a temporary partition separating and empty compartment with the medical bag from the concentrate while the predetermined amount of fluid is transferred into the interior volume and dropping the fluid into the concentrate. In some embodiments, the method may further comprise further mixing the fluid with the concentrate by compromising a second temporary partition in the interior volume while the predetermined amount of fluid is transferred into the medical bag and dropping the fluid and concentrate to an end of the bag opposite the port. In some embodiments, rotating the medical bag may further comprise dissolving a concentrate within the medical bag into the predetermined amount of fluid. In some embodiments, positioning the medical bag in the first position may comprise positioning the port at least partially into an airflow channel surrounding the fluid dispensing sharp. In some embodiments, the method may further comprise flowing air through the airflow channel. In some embodiments, the method may further comprises providing a laminar flow of clean air to the airflow channel. In some embodiments, the at least one antimicrobial light emitter includes a UVC LED. In some embodiments, positioning the medical bag in the first position may comprise displacing the port within an irradiation transmissive barrier of a fluid dispensing cartridge. In some embodiments, the method may further comprise illuminating at least a portion of the fluid dispensing sharp with the at least one antimicrobial light emitter. In some embodiments, the method may further comprise mixing the predetermined amount of fluid with a concentrate within the medical bag for a solution selected from a list consisting of a saline solution, a sugar solution, a saline and sugar solution, normal saline, half normal saline, D5W, peritoneal dialysate, hemodialysis dialysate, Ringer's solution, lactated Ringer's solution, Hartmann's Solution, hypotonic solution, isotonic solution, and hypertonic solution. In some embodiments, the method may further comprise mixing the predetermined amount of fluid with a concentrate within the medical bas selected from a list consisting of, powder, lyophilized medical agent, crystalline concentrate, salt concentrate, sodium chloride concentrate, sugar concentrate, and dextrose concentrate. In some embodiments, positioning the medical bag in at least one of the first position and second position may comprise directing the port with at least one guide. In some embodiments, dispensing the antimicrobial agent may comprise one of spraying and atomizing the antimicrobial agent. In some embodiments, dispensing the antimicrobial agent may comprise dispensing isopropyl alcohol. In some embodiments, dispensing the antimicrobial agent may comprise drawing the agent from a removable antimicrobial agent source reservoir.

In accordance with another example embodiment of the present disclosure, a fluid dispensing cartridge for a medical fluid dispenser may comprise a midbody having a filter support, a first side, and second side. The first side may include a first set of ribs, inlet walls, and a first filter support perimeter wall extending therefrom. The second side may include a second set of ribs, outlet walls, and a second filter support perimeter wall extending therefrom. The cartridge may further comprise a first cover body coupled to the first set of ribs, the inlet walls, and the first filter support perimeter wall. The first cover body may cooperate with the inlet walls to form an inlet flow path. The cartridge may further comprise a filter disposed intermediate the filter support and a filter capture projection of the first cover body. The cartridge may further comprise a second cover body coupled to the second set of ribs, the outlet walls, and the second filter support perimeter wall. The second cover body may cooperate with the outlet walls to form an outlet flow path. The cartridge may further comprise an irradiation transmissive barrier. The cartridge may further comprise an airflow channel defined by the midbody, at least one of the first and second cover body, and the irradiation transmissive barrier. The cartridge may further comprise a dispensing sharp coupled to a hub within the airflow channel. The dispensing sharp may be in fluid communication with the outlet flow path. The cartridge may further comprise a frangible bearing connection port in fluid communication with the inlet flow path.

In some embodiments, the dispensing sharp may be a pencil tipped sharp with two side ports disposed in opposition to one another. In some embodiments, the first and second cover body may be coupled to the midbody via laser weld. In some embodiments, the first and second cover body may be formed of transparent polymer and the midbody may be formed of a dark polymer. In some embodiments, the airflow channel may vary in cross-sectional area along the length of the airflow channel. In some embodiments, the irradiation transmissive barrier may be at least partially transparent to UVC light. In some embodiments, the irradiation transmissive barrier may be formed of fused quartz. In some embodiments, the airflow channel may extend along a straight line path through the housing and has a round cross-sectional shape. In some embodiments, the irradiation transmissive barrier may be captured within a barrier retention assembly defined by the midbody and at least one of the first and second cover body. In some embodiments, the connection port may have a first section having a first diameter and a second section having a second diameter smaller than the first. The second section may extend to a terminal end of the connection port and at least one sealing member may be disposed on the exterior surface of each of the first second and second section. In some embodiments, the outlet flow path may extend into communication with the filter support through a top portion of the second filter support perimeter wall. In some embodiments, the inlet walls may be continuous with the first filter support perimeter wall and the outlet walls are continuous with the second filter support perimeter wall. In some embodiments, the filter may be a 0.2 μm filter. In some embodiments, the filter support may be a grate. In some embodiments, the filter capture projection may include a sealing member on an end of the filter capture projection most distal to a main portion of the first cover body and a support wall aperture in fluid communication with the inlet flow path.

In accordance with another example embodiment of the present disclosure, a fluid dispensing cartridge for a medical fluid dispenser may comprise a housing assembly having a flow path extending therethrough. The cartridge may further comprise an inlet port. The cartridge may further comprise an irradiation transmissive barrier coupled to the housing assembly. The cartridge may further comprise an airflow channel extending through the housing assembly. The irradiation transmissive barrier may define at least a portion of the airflow channel. The cartridge may further comprise a dispensing sharp coupled to a hub within the airflow channel. The cartridge may further comprise a filter assembly captured within the housing. The cartridge may further comprise a housing sealing member. The cartridge may further comprise a cover coupled to the housing and an end of the cover compressing the housing sealing member and establishing a sealed volume surrounding the airflow channel.

In some embodiments, the dispensing sharp may be a pencil tipped sharp with two side ports disposed in opposition to one another. In some embodiments, the airflow channel may vary in cross-sectional area along the length of the airflow channel. In some embodiments, the irradiation transmissive barrier may be at least partially transparent to UVC light. In some embodiments, the airflow channel may extend along a straight line path through the housing and has a round cross-sectional shape. In some embodiments, a frangible may be coupled to a terminal end of the inlet port. There may be a plurality of sealing members disposed on the exterior surface of the inlet port. In some embodiments, the at least one filter may include a 0.2 μm filter. In some embodiments, a catch may extend from an exterior surface of the cover. In some embodiments, the cover may be engaged to the housing via a snap fit. In some embodiments, an end of the airflow channel may include at least one medical reservoir port guide.

In accordance with another example embodiment of the present disclosure, a fluid dispensing cartridge for a medical fluid dispenser may comprise a first body having an inlet flow path extending therethrough. The cartridge may further comprise a second body having an outlet flow path extending therethrough. The cartridge may further comprise a third body including a protruding central region surrounded by a peripheral region. The protruding central region may include a hub and an airflow channel segment. A supply flow path may extend through the third body to the hub. The cartridge may further comprise an inlet port. The cartridge may further comprise a filter assembly captured between the first and second body. The cartridge may further comprise a dispensing sharp coupled to the hub. The cartridge may further comprise an irradiation transmissive barrier. The cartridge may further comprise an airflow channel extending through the cartridge. The airflow channel segment and irradiation transmissive barrier may define at least a portion of the airflow channel. One end of the airflow channel may include at least one port guide. The cartridge may further comprise a sealing member coupled to the peripheral region.

In some embodiments, the dispensing sharp may be a pencil tipped sharp with at least one side port. In some embodiments, the airflow channel may vary in cross-sectional area along the length of the airflow channel. In some embodiments, the irradiation transmissive barrier may be at least 75% transmissive to UVC light. In some embodiments, the airflow channel may extend along a straight line path through the cartridge. In some embodiments, at least a first and second sealing member may be disposed on the exterior surface of the inlet port. A frangible may be coupled to a terminal end of the inlet port. The inlet port may be monolithically formed with the first body. In some embodiments, the irradiation transmissive barrier may be captured between the airflow channel segment and a medical reservoir port guide coupled to the third body on at an end of the irradiation transmissive barrier opposite the air flow channel segment. In some embodiments, the airflow channel may be sealed within a cap. In some embodiments, a catch may extend from an exterior surface of the cap. In some embodiments, the cap may be engaged to the third body via a snap fit. In some embodiments, the sealing member may be compressed against an end of the cap. In some embodiments, a peripheral most surface of the sealing member may be uncovered by the end of the cap and surrounds the end of the cap.

In accordance with another exemplary embodiment of the present disclosure a method of installing a fluid dispensing cartridge in a fill assembly for a medical reservoir may comprise placing a cartridge in a cartridge dock. The method may further comprise displacing housing of a cover removal assembly to a sealing position in which a housing sealing member of the housing is sealed around a fenestration in a processing compartment wall on a first side of the processing chamber wall. The method may further comprise displacing the cartridge dock to an access position in which an exposed region of a cover of the cartridge is displaced into an interior bay of the housing and a protrusion at an end of the exposed region is compressed against the housing sealing member. The method may further comprise establishing a seal around the fenestration on the second side of the processing compartment wall with a cartridge sealing member. The method may further comprise actuating a cover engagement assembly within the housing into engagement with the cover of the cartridge. The method may further comprise removing the cover from the cartridge by displacing the housing away from the cartridge.

In some embodiments, the method may further comprise supplying a negative pressure to the interior bay. In some embodiments, the method may further comprise verifying that the negative pressure is stable and at least at a negative pressure set point. In some embodiments, the method may further comprise displacing a clean air supply outlet against an end of an air channel extending through the cartridge. In some embodiments, the method may further comprise displacing an end of a priming line over a dispensing sharp of the cartridge. In some embodiments, the method may further comprise flowing a volume of priming fluid through the cartridge into the priming line. In some embodiments, the method may further comprise irradiating the end of the priming line with at least a predefined dose of UVC light. In some embodiments, actuating the cover engagement assembly may comprise driving a hook of the cover engagement assembly into a catch on the cover. In some embodiments, the method may further comprise positioning an inlet port of the cartridge in a first position within a receptacle of a supply manifold and passing a disinfection fluid through the receptacle and out of a drain flow path in communication with the receptacle. In some embodiments, the method may further comprise compromising a frangible of the inlet port with a spike in the receptacle and blocking flow from the spike to the drain flow path with a sealing member on the inlet port. In some embodiments, actuating the cover engagement assembly may comprise exerting a force on the cover which pulls the protrusion of the cover against the housing sealing member.

In accordance with another example embodiment of the present disclosure a filling assembly for medical reservoirs may comprise a compartment having a wall with a fenestration. The filling assembly may further comprise a cartridge loading assembly on a first side of the wall including fluid dispensing cartridge dock coupled to a dock actuator and displaceable via the dock actuator toward and away from the fenestration. The filling assembly may further comprise a cartridge cover removal assembly on a second side of the wall including a cover retainer coupled to a retainer actuator and displaceable via the retainer actuator toward and away from the fenestration. The cover retainer may define a bay with a cover engagement assembly disposed therein. The filling assembly may further comprise an air handling assembly in fluid communication with the compartment. The air handling assembly may include an air output magnetically coupled to an air output actuator through the wall of the compartment and displaceable between a retracted position and a deployed position in which the air output is at least partially within the compartment.

In some embodiments, the filling assembly may further comprise an irradiation assembly coupled to an irradiation assembly actuator on the second side of the wall. The irradiation assembly may be displaceable via the irradiation assembly actuator toward and away from the fenestration. In some embodiments, the irradiation assembly may include a plurality of UVC LEDs. In some embodiments, the filling assembly may further comprise a fluid supply manifold coupled to a supply manifold actuation assembly on the first side of the wall. In some embodiments, the cover retainer may include a sealing member surrounding an ingress to the bay. The sealing member may compress against the wall directly surrounding the fenestration when the cover retainer is displaced toward the fenestration to an end of its displacement range. In some embodiments, the ingress to the bay may have a cross-sectional area smaller than the cross-sectional area of the fenestration. A portion of the sealing member may be accessible through the fenestration from the first side of the wall when the cover retainer is at the end of its displacement range. In some embodiments, the cover retainer may include a negative pressure inlet coupled to a negative pressure source and a pressure sensor in fluid communication with the bay. In some embodiments, the cover removal assembly may include a hook operatively coupled to a pneumatic actuator and displaceable between a raised position and a deployed position. In some embodiments, the air handling assembly may include a blower, at least one filter, and at least one flow laminarizer. In some embodiments, the air output may include an internal bore having a variable cross-sectional area. In some embodiments, the air output may include an exterior sleeve having a first and second sleeve magnet coupled thereto and the air output actuator may include a yoke surrounding an air output guide formed by the wall. The yoke may include a first and second yoke magnet which are respectively aligned with the first and second sleeve magnets. In some embodiments, the air output may include an exterior sleeve with a magnetic body coupled thereto and at least one magnetic position sensor is disposed on an exterior of an air output guide formed by the wall. In some embodiments, the cartridge dock may comprise at least one cartridge detector. In some embodiments, each of the at least one cartridge detector may be selected from a group consisting of an optical sensor, a microswitch, an imager, a beam break sensor, and a reflective sensor. In some embodiments, the cartridge dock may include an asymmetric cartridge receptacle. In some embodiments, the filling assembly may include a fill displacement stage including a set of medical bag port retainers. The fill displacement stage may be coupled to a stage actuator and displaceable toward and away from the fenestration via the stage actuator.

In accordance with another example embodiment of the present disclosure a medical bag individualizing assembly for separating a medical bag from a chain of medical bags may comprise an actuator. The bag individualizing assembly may further comprise a displacement stage coupled to the actuator and displaceable through a displacement range via the actuator. The bag individualizing assembly may further comprise a splitter coupled to the displacement stage. The splitter may have a feed end and an opposing trailing end. The feed end may define a feed guide leading to a feed channel with first and second feed rollers on opposing sides thereof. The feed channel may furcate to a first and second branch which extend on opposite sides of a set of coaxial separating rollers and to the trailing end of the splitter. The first branch may be open to an upstream side of the splitter and the second branch may be open to a downstream side of the splitter.

In some embodiments, the feed guide may be defined by two oppositely sloped ramps at the feed end of the splitter. In some embodiments, the first and second feed roller may extend parallel to one another. In some embodiments, the set of separating rollers may include two separating rollers disposed abreast of one another on a common pivot bearing. In some embodiments, the set of separating rollers may include two separating rollers each of which extending across half of the feed channel. In some embodiments, each of the first and second branch may extend across 70-90% of the width of the splitter. In some embodiments, the actuator may include a ballscrew coupled to an electromechanical motor. In some embodiments, the medical bag individualizing assembly may further comprise a controller in data communication with the actuator. The actuator may be powered based on commands from the controller and the controller may generate a fault when a current draw from the electromechanical motor exceeds a threshold.

In accordance with another example embodiment of the present disclosure a method of mixing fluid in a medical bag may comprise placing a bag in a cradle of a bag retention assembly. The method may further comprise grasping ports of the bag with a grasper of the bag retention assembly. The method may further comprise closing a set of doors of the bag retention assembly. The method may further comprise rotating the bag retention assembly in opposing directions between preset pairs of rotational orientations. The method may further comprise dwelling in each preset rotational orientation for a dwell interval specific to each pair of positions.

In some embodiments, the method may further comprise dissolving a solid concentrate selected from a list consisting of powder, lyophilized medical agent, crystalline concentrate, salt concentrate, sodium chloride concentrate, normal saline concentrate, half normal saline concentrate, sugar concentrate, dextrose concentrate, a saline and sugar solution concentrate, D5W concentrate, peritoneal dialysate concentrate, hemodialysis dialysate concentrate, Ringer's solution concentrate, lactated Ringer's solution concentrate, and Hartmann's solution concentrate. In some embodiments, the method may further comprise waiting a predefined period of time and analyzing the medical bag for the presence of particulates. In some embodiments, the method further may comprise reading an indicium on the medical bag and selecting a motion profile based on data read from the indicium. The motion profile may define the preset pairs of rotational orientations. In some embodiments, rotating the bag retention assembly may comprise rotating the bag retention assembly at a rotation rate specific to each pair of positions. In some embodiments, rotating the bag retention assembly may comprise rotating the bag retention assembly between each pair of preset positions a number of times specific to each pair of positions. In some embodiments, rotating the bag retention assembly may comprise inverting the bag retention assembly at least once. In some embodiments, each rotational orientation in each pair of rotational positions may be equal in magnitude from a starting orientation and opposite in direction. In some embodiments, closing the set of doors on the medical bag may comprise depressing a central region of the medical bag and displacing fluid laterally within the medical bag. In some embodiments, closing the set of doors on the medical bag may comprise contacting the medical bag with a gripping material on a surface of the doors. In some embodiments, the method may further comprise collecting a plurality of images of the medical bag and analyzing the plurality of images for indications of undissolved concentrate. The method may further comprise rotating the bag retention assembly in opposing directions at between at least one preset pair of rotational orientations when at least one indication of undissolved concentrate is detected. In some embodiments, closing the doors may comprise monitoring the position of the doors and generating a fault notification with a controller when at least one of the doors reaches an overly closed position.

In accordance with another example embodiment of the present disclosure a medical reservoir mixing assembly may comprise a bag retention assembly including a cradle and a reservoir holder assembly. The mixing assembly may further comprise a rotary actuator coupled the bag retention assembly. The mixing assembly may further comprise a rotation sensor configured to output a data signal indicative of the rotational orientation of the bag retention assembly. The mixing assembly may further comprise a controller in data communication with the rotation sensor and the rotary actuator and configured to orchestrate displacement of the bag retention assembly via the rotary actuator in opposing rotational directions between preset pairs of rotational orientations. The controller may be further configured to wait for a dwell timer to elapse at each rotational orientation.

In some embodiments, the reservoir holder assembly may include a set of doors each coupled to at least one door actuator. In some embodiments, the reservoir holder assembly may include a set of doors. Each door may have an inner face and an exterior face. At least a portion of the inner face may be covered with a gripping material. In some embodiments, the reservoir holder assembly may include a set of doors displaceable between an open state and a closed state. Each door may include a set of projections. The projections of the doors may interdigitate when the doors are in a closed state. In some embodiments, the projections may be more proximal a reservoir rest surface of the cradle than a section of the door from which they extend when the doors are in the closed state. In some embodiments, the reservoir holder assembly may include a set of IV bag port grippers actuatable between an open state and a closed state. In some embodiments, the mixing assembly may further comprise a reservoir indicium reader. The controller may be in data communication with the indicium reader and may select a motion profile defining the preset pairs rotational orientations based on data from the indicium reader. In some embodiments, the controller may be further configured to orchestrate displacement of the bag retention assembly at a rotation rate specific to each pair of positions of the preset pairs of positions. In some embodiments, the controller may be further configured to orchestrate displacement of the bag retention assembly to each preset pair of positions a number of times specific to each pair of positions. In some embodiments, one of the preset pairs of positions may orient the bag retention assembly in an inverted position. In some embodiments, each rotational orientation in each pair of rotational positions may be equal in magnitude from a starting orientation and opposite in direction. In some embodiments, the cradle may be illuminated and may include a pattern of light and dark regions. The mixing assembly may further comprise an external illuminator and at least one imager with a field of view at least partially encompassing the bag retention assembly.

In accordance with another example embodiment of the present disclosure a particulate inspection system for detecting and classifying contents of interest within a medical reservoir may comprise a cradle including a reservoir rest and a reservoir retainer. The cradle may include a backlight. The rest body may be in an illumination field of the backlight and may include a pattern of light and dark regions. The system may further comprise an external illuminator including at least light emitter. The system may further comprise a vision assembly disposed opposite the cradle. The vision assembly may include at least one imager having a field of view encompassing at least a portion of the cradle. The system may further comprise a controller in data communication with each of the at least one imager. The controller may be configured to command capture of a series of images from each of the at least one imager, receive the images, pre-process the images into processed images, detect regions of interest within the processed images, analyze the regions of interest, and classify the regions of interest into at least a first content type and second content type based on the analysis.

In some embodiments, the system may further comprise a rotary actuator coupled to the cradle. In some embodiments, the backlight may emit white light. In some embodiments, the pattern may be a chessboard pattern. In some embodiments, the reservoir retainer may be a set of IV bag port graspers actuatable between an open state and port retaining state. In some embodiments, the external illuminator may be an underlight. In some embodiments, the at least one light emitter may include a plurality of LEDs electrically coupled in series. In some embodiments, each of the at least one light emitter may be a collimated light emitter. In some embodiments, each of the at least one light emitter may emit light in a narrow beam angle less than 20°. In some embodiments, the external illuminator may include a polarizer. In some embodiments, each of the at least one image may include a polarizing filter on a lens thereof. In some embodiments, the vision system may include a plurality of imagers. Each of the field of view of each imager may overlap the field of view of at least one other imager of the vision system. In some embodiments, the optical axis or viewing axis each of the at least one imager may be oriented at an angle 30-35° from a plane extending perpendicular to the height axis of the cradle. In some embodiments, the controller may be further configured to assign each region of interest in each of the processed images to one or more track. Each of the one or more track may define the displacement of an associated region of interest over the series of images. In some embodiments, for each track, the controller may be configured to extract an extracted portion of every processed image in which the associated region of interest is detected. The extracted portion may include the associated region of interest. The controller may be configured to analyze the regions of interest by analyzing the extracted portions of the processed images for each track. In some embodiments, the controller may be configured to analyze the regions of interest with a convolutional neural network. In some embodiments, the controller may be configured generate a reservoir acceptability determination. The acceptability determination may be indicated as a failure if any contents of interest are classified as the second content type. In some embodiments, the first content type may be a gas bubble and the second content type is anything other than a gas bubble. In some embodiments, the medical reservoir may be an IV bag.

In accordance with another example embodiment of the present disclosure a method of inspecting a medical reservoir may comprise placing the medical reservoir in a cradle. The method may further comprise providing a background on first side of the medical reservoir which contrasts between light and dark. The method may further comprise illuminating the medical reservoir with an illumination assembly separate from the cradle. The method may further comprise collecting at least one series of frames of the medical reservoir from at least one imager on a second side of the medical reservoir opposite the first. The method may further comprise processing each of the frames in the at least one series of images into a series of processed frames. The method may further comprise detecting regions of interest within each of the processed frames of the series of processed frames. The method may further comprise associating each of the detected regions of interest with tracks. Each track may define the displacement of a specific tracked region of interest over a plurality of processed frames. The method may further comprise analyzing the specific tracked region of interest in each processed frame for each track. The method may further comprise classifying the specific tracked region of interest for each track as one of at least a first content type and a second content type.

In some embodiments, placing the medical reservoir in the cradle may comprise placing an IV bag in the cradle. In some embodiments, the method may further comprise agitating the medical reservoir. In some embodiments, providing the background may comprise placing a pattern of light and black regions on a rest panel of the cradle. In some embodiments, providing the background may comprise adjusting a backlight on the first side of the medical reservoir to provide a background which provides a temporal contrast between light and dark. In some embodiments, illuminating the medical reservoir may comprise directing collimated light at the medical reservoir from an underlight. In some embodiments, illuminating the medical reservoir may comprise directing polarized light at the medical reservoir. In some embodiments, the at least one imager may include a plurality of imagers each having a field of view which at least partially overlaps that of at least one other imager of the plurality of imagers and collecting the at least one series of frames may comprise collecting a plurality of series of frames. Each plurality of series of frames may be captured from a respective imager of the plurality of imagers. In some embodiments, the method may further comprise providing a polarizing filter for each of the at least one imager. In some embodiments, processing each of the frames may comprise at least one of smoothing each of the frames, generating a foreground segmented image, generating a near edge filtered foreground segmented image based on an edge detection analysis of each frame, performing at least one kernel convolution, performing at least one morphological transformation, and removing pixel clusters having yielding a contour outside of a predefined size range. In some embodiments, detecting regions of interest within each of the processed frames may comprise generating a bounding box around each pixel cluster of interest remaining in the processed frames. In some embodiments, associating each of the detected regions of interest with tracks may comprise determining track predictions for each region of interest in a starting frame and matching the track predictions to detected regions of interest in a subsequent frame. In some embodiments, analyzing the specific tracked region of interest may comprise generating a region of interest score for the specific tracked region of interest in each processed frame of each track. In some embodiments, analyzing the specific tracked region of interest may comprise feeding an extracted portion of each processed frame of each track to a convolutional neural network. Each extracted portion of each processed frame including the specific tracked region of interest. In some embodiments, classifying the specific tracked region of interest for each track as one of at least the first content type and the second content type may further comprise classifying the specific tracked region of interest as at least one content subtype. In some embodiments, the method may further comprise generating an annotated image. In some embodiments, generating the annotated image may comprise depicting a representation of the displacement path of each specific tracked region of interest on a selected frame from each of the at least one series of frames.

In accordance with another example embodiment of the present disclosure, a method of inspecting a medical reservoir may comprise agitating the medical reservoir. The method may further comprise illuminating the medical reservoir with an external illuminator. The method may further comprise illuminating a patterned background on a first side of the reservoir. The method may further comprise capturing a plurality of raw frames of the medical reservoir from a second side of the medical reservoir. The method may further comprise processing, via at least one processor, the plurality of raw frames to create processed frames. The method may further comprise analyzing, via the at least one processor, the processed frames to identify detected pixel clusters of interest. The method may further comprise associating, via the at least one processor, the detected pixel clusters of interest with tracks indicative of the displacement of each pixel cluster of interest over the plurality of frames. The method may further comprise classifying, via the at least one processor, the pixel clusters of interest associated with each track as one of at least a first content type and a second content type.

In some embodiments, agitating the medical reservoir may comprise rotating the medical reservoir through a series of rotational orientations specified in a motion profile. In some embodiments, the method further may comprise generating a log including at least the raw frames and classifications for the pixel clusters of interest associated with each track and communicating the log to an external database. In some embodiments, illuminating the medical reservoir with the external illuminator may comprise directing collimated, polarized light in a preset wavelength range at the medical reservoir. In some embodiments, illuminating the medical reservoir with the external illuminator may comprise emitting light from a plurality of light emitters at the reservoir. Each light emitter may emit light in a narrow beam angle less than 20°. In some embodiments, illuminating the patterned background may comprise illuminating a rest surface for the medical reservoir. The rest surface may include a checkered pattern of light and dark regions. In some embodiments, capturing the plurality of raw frames may comprise capturing the plurality of raw frames with a plurality of imagers. Each imager may have a field of view which overlaps with the field of view of at least one other imager of the plurality of imagers. In some embodiments, processing the raw frames may comprise at least of generating a foreground mask, detecting edges in the raw frame, generating near edge filtered foreground segmented image from each raw frame, performing at least one morphological transformation, performing at least one kernel convolution, and performing a dilation convolution. In some embodiments, analyzing the processed frames to identify detected pixel clusters of interest may comprise at least one of identifying pixel clusters yielding a contour size outside of a predefined range, identifying pixel clusters which fail at least one predefined validity criteria, performing at least one morphological transformation, performing at least one kernel convolution, performing an erosion convolution, identifying pixel clusters which fail at least one predefined validity criteria, defining bounding boxes around pixel clusters of interest. In some embodiments, processing the raw frames may comprise generating a foreground segmented image from each raw frame, detecting edges in each raw frame and generating an edge image for each raw frame, generating a dilated edge image for each raw frame, generating a near edge filtered foreground segmented image from the foreground segmented image for each raw frame using the respective dilated edge image for each raw frame, and dilating each near edge filtered foreground segmented image. In some embodiments, analyzing the processed frames to identify detected pixel clusters of interest may comprise identifying pixel clusters in each dilated near edge filtered foreground segmented image which fail at least one predefined validity criteria, generated an eroded image from each dilated near edge filtered foreground segmented image in which the identified pixel clusters have been removed, defining bounding boxes around pixel clusters of interest in each eroded image. In some embodiments, associating the detected pixel clusters of interest with tracks may comprise determining track predictions for each detected pixel cluster of interest in a starting frame and matching the track predictions to detected pixel clusters of interest in a subsequent frame. In some embodiments, classifying the pixel clusters of interest associated with each track may comprise generating at least one score for the detected pixel clusters of interest associated with each track. In some embodiments, generating the at least one score may comprise generating at least one of a color score, a trajectory score, a shape score, and a sharpness score. In some embodiments, classifying the pixel clusters of interest associated with each track may comprise classifying the pixel clusters of interest associated with each track with a convolutional neural network trained on images of medical reservoirs with known contents. In some embodiments, classifying the pixel clusters of interest associated with each track may comprise classifying the pixel regions of interest associated with each track as at least one content subtype. In some embodiments, the method may further comprise generating an annotated image. In some embodiments, generating the annotated image may comprise depicting a representation of the displacement path of the pixel cluster of interest associated with each track on a selected frame of the plurality of raw frames. In some embodiments, the method may further comprise generating a pass indication for the medical reservoir when no pixel clusters of interest are classified as the second type and generating a fail indication for the medical reservoir when at least one pixel cluster of interest is classified as the second type. In some embodiments, the method may further comprise generating a troubleshooting suggestion when the fail indication is generated. In some embodiments, the method may further comprise detecting a pattern indicative of the patterned background being distorted by an air bubble in one of the plurality of raw frames and processed frames. In some embodiments, the medical reservoir may be a bag.

In accordance with another example embodiment of the present disclosure a particulate inspection system for detecting and classifying contents of interest within a medical reservoir may comprise a reservoir rest body having a pattern of light and dark regions. The system may further comprise a rest body backlight. The system may further comprise an external illuminator. The system may further comprise a vision assembly opposite the rest body. The vision assembly may include at least one imager. Each imager may have a field of view encompassing at least a portion of rest body. The system may further comprise a controller in data communication with each of the at least one imager. The controller may be configured to command capture of a series of images from each of the at least one imager, receive the images, process the images into processed images, detect regions of interest within the processed images, analyze the regions of interest, and classify the regions of interest as one of a bubble and something other than bubble.

In some embodiments, the system may further comprise a rotary actuator coupled to the rest body. In some embodiments, the rest body backlight may emit white light. In some embodiments, the pattern may be a checkered pattern. In some embodiments, the system may further comprises a reservoir retainer container coupled to reservoir rest body. The reservoir retainer may include a set of IV bag port graspers actuatable between an open state and port retaining state. In some embodiments, the external illuminator may be an underlight. In some embodiments, the external illuminator may include a plurality of LEDs electrically coupled in series. In some embodiments, the external illuminator may include a plurality collimated light emitters with a narrow beam angle less than 20°. In some embodiments, the external illuminator may include a polarizer. In some embodiments, each of the at least one imager may include a polarizing filter on a lens thereof. In some embodiments, the vision system may include a plurality of imagers. Each field of view of each imager may overlap the field of view of at least one other imager of the vision system. In some embodiments, the view axis each of the at least one imager is oriented at an angle 30-35° from a plane extending perpendicular to the height axis of the rest body. In some embodiments, the controller may be further configured to assign each region of interest in each of the processed images to one or more track. Each of the one or more track may define the displacement of an associated region of interest throughout the plurality of processed images. In some embodiments, for each track, the controller may be configured to extract an extracted portion of every processed image in which the associated region of interest is detected. The extracted portion may include the associated region of interest. The controller may be configured to analyze the regions of interest by analyzing the extracted portions. In some embodiments, the controller may be configured to analyze the regions of interest with a convolutional neural network. In some embodiments, the controller may be configured generate a reservoir acceptability determination. The acceptability determination may be indicated as a failure if any contents of interest are classified as something other than a bubble. In some embodiments, the medical reservoir may be an IV bag.

In accordance with another example embodiment of the present disclosure a method of capturing images of a medical reservoir for inspection may comprise positioning a first side of the medical reservoir against a reservoir rest. The method may further comprise providing, with a first illuminator, a contrasting background for the medical reservoir. The method may further comprise illuminating the medical reservoir with a second illuminator which emits collimated, polarized light in at least one predefined wavelength range. The method may further comprise capturing a plurality of images of the medical reservoir with at least one imager on a second side of the medical reservoir opposite the first side. Each of the at least one imager being associated with a polarizing filter.

In some embodiments, positioning the first side of the medical reservoir against the reservoir rest may comprise retaining the medical reservoir with a retainer assembly. In some embodiments, the medical reservoir may be an IV bag and retaining the medical reservoir may comprise retaining one or more port of the IV bag in one of a passive retainer and a set of grasper jaws. In some embodiments, the method may further comprise agitating the medical reservoir. In some embodiments, agitating the medical reservoir may comprise rotating the medical reservoir between at least one pair of predefined rotational orientations. In some embodiments, the method may further comprise waiting a dwell period after agitating the medical reservoir before capturing the plurality of images. In some embodiments, providing the contrasting background may comprise backlighting the reservoir rest. The reservoir rest may include a pattern of light and dark regions. In some embodiments, the pattern of light and dark regions may be a checker pattern. In some embodiments, providing the contrasting background may comprise adjusting the output of the first illuminator over time. In some embodiments, illuminating the medical reservoir with a second illuminator may comprise under lighting the medical reservoir with the second illuminator. In some embodiments, one of the at least one predefined wavelength range may be selected from a list consisting of 580-620 nm and 620-750 nm. In some embodiments, the at least one imager may include a plurality of imagers. A field of view of each of the plurality of imagers may overlap the field of view at least one other imager of the plurality of imagers. In some embodiments, the field of view of each of the plurality of imagers may overlap the field of view at least one other imager of the plurality of imagers by at least 10%. In some embodiments, each of the plurality of imagers may be oriented with a view axis at an angle 30-35° from a plane extending perpendicular to the height axis of the reservoir rest. In some embodiments, capturing a plurality of images of the medical reservoir with at least one imager on a second side of the medical reservoir may comprise capturing the plurality of images at a frame rate of at least 8 frames per second from each of the at least one imager.

In accordance with another example embodiment of the present disclosure a marking assembly for placing a marking on a medical reservoir may comprise an embossing head having a plurality of individually displaceable segments on a shaft. The embossing head may further comprise a plurality of marking bodies. Each segment may include at least one of the plurality of marking bodies raised from and exterior surface thereof. A first end segment of the plurality of segments may be fixed with relation to the shaft. The marking assembly may further comprise a plurality of clutches. Each of the plurality of segments excepting the end segment may be paired with a respective one of the plurality of clutches. The marking assembly may further comprise a plurality of relative rotation limiters. A first of the relative rotation limiters may be formed between the trunnion and a second end segment of the plurality of segments. The second end segment may be opposite the first end segment. The remainder of the relative rotation limiters may be formed between each of the plurality of segments. The marking assembly may further comprise a trunnion. The shaft may be rotatably coupled to the trunnion. The marking assembly may further comprise a rotary actuator coupled to the shaft. The marking assembly may further comprise a rotation sensor configured to output a data signal indicative of the rotational position of the shaft. The marking assembly may further comprise a marking actuator coupled to the trunnion. The trunnion and embossing head may be displaceable between a retracted state and an embossing state via the marking actuator.

In some embodiments, the assembly may be coupled to the shaft through a transmission. In some embodiments, the transmission may comprise a timing belt. In some embodiments, each of the individual segments may be a rotor with a polygonal cross-section. In some embodiments, each of the individual segments may include a plurality of faces. There may be at least one marking body raised from at least one of the plurality of faces of each segment. In some embodiments, the marking bodies may be at least one of a list consisting of alphabetic characters, numerical characters, punctuation characters, pictograms, and symbols. In some embodiments, the plurality of clutches may be slip clutches. In some embodiments, each of the plurality of clutches may include a member biased radially outward from the shaft against a surface of a shaft aperture in the paired segment and the surface of the shaft aperture includes a plurality of detents. In some embodiments, each of the plurality of clutches may be disposed within the segment with which it is paired and includes a member biased axially against the surface of an adjacent segment of the plurality of individual segments, the surface of the adjacent segment including a plurality of detents. In some embodiments, the first relative rotation limiter may be formed by a projection on one of the trunnion and the second end segment which rides along an interrupted annular channel in the other of the trunnion and the second end segment. In some embodiments, the remainder of the relative rotation limiters may be formed by a projection of one segment of the plurality of segments extending into an interrupted annular channel in and adjacent segment of the plurality of segments. In some embodiments, each segment may include an equal number of exterior faces. Each interrupted annular channel may have an interrupt gap over an arc of 360° divided by the number of exterior faces. In some embodiments, the marking assembly may further comprise a verification imager and a controller. The controller may be configured to command capture of a marking formed by the marking assembly and perform optical character recognition on the marking to determine a marking content. The controller may be configured to compare the marking content to an expected marking and generate a fault in the event the marking content does not conform to the expected marking. In some embodiments, the marking actuator may be pneumatic. In some embodiments, the marking assembly may further comprise a backstop assembly including a compliant body opposite the embossing head. In some embodiments, the compliant body may be coupled into a backstop receptacle having a depth less than a distance each of the marking bodies is raised from the segments. The receptacle may have a footprint with a width dimension less than the width dimension of the embossing head. In some embodiments, each of the individual segments may include a plurality of faces. There may be at least one marking body raised from at least one of the plurality of faces on each segment. The compliant body may be coupled into a backstop receptacle having a depth less than a distance each of the marking bodies is raised from the segments. The receptacle may have a footprint with a height dimension less than the height dimension of each of the plurality of faces but greater than a largest height dimension of any of the marking bodies. In some embodiments, the backstop assembly may further comprise a reservoir guide. The reservoir guide may have a reservoir introduction opening. The reservoir guide may define a reservoir receiving channel which tapers thinner in width as distance from the reservoir introduction opening increases.

In accordance with another example embodiment of the present disclosure a method of marking a medical reservoir may comprise displacing a portion of a reservoir into a guide and aligning a marking region of the reservoir with a complaint body. The method may further comprise aligning a first desired string of marking bodies of an embossing head in a marking position. The method may further comprise driving the embossing head against the reservoir such that the marking region is compressed between the compliant body and the embossing head. The method may further comprise imaging the marking region. The method may further comprise analyzing the marking region to determine a marking content present in the marking region. The method may further comprise verifying correctness of the marking content by comparing the marking content to the desired string of characters. The method may further comprise aligning a second desired string of marking bodies of the embossing head in the marking position and driving the embossing head against the reservoir such that the marking region is compressed between the compliant body and the embossing head when the marking content does not match the desired string of marking bodies.

In some embodiments, the reservoir may be an IV bag and the marking region is in a peripheral seal of the IV bag. In some embodiments, driving the embossing head against the reservoir may comprise holding the marking region in compression between the compliant body and the embossing head for a dwell period. In some embodiments, the method may further comprise storing the marking content in a log and communicating the log to an external database. In some embodiments, aligning the first desired string of marking bodies may comprise rotating a shaft of the embossing head to which a first rotor of the embossing head is fixed coupled in a first direction until a relative rotation limiter for another rotor is engaged. In some embodiments, the aligning the first desired string of marking bodies comprises continuing to rotate the shaft in the first direction until a clutch between two rotors of the embossing heads slips at least once. In some embodiments, aligning the first desired string of marking bodies may comprise rotating the shaft in a second direction opposite the first direction.

In accordance with another example embodiment of the present disclosure a dispenser of medical reservoirs may comprise a housing having an outlet. The dispenser may further comprise a removable cover sealing the outlet. The dispenser may further comprise a reel rotatably coupled within the housing. The dispenser may further comprise a chain of abreastly coupled medical bags coupled to the reel. The dispenser may further comprise a bag holder with a set of port retainers coupled thereto. A set of ports of a lead bag of the chain may be retained in the set of port retainers.

In some embodiments, each of the medical bags may contain at least one concentrate selected from a list consisting of a saline brine, a sodium chloride brine, a sugar solution, a saline and sugar solution, a peritoneal dialysate precursor solution, a hemodialysis precursor solution, a Ringer's solution concentrate, lactated Ringer's solution concentrate, and Hartmann's solution concentrate, a powder, a lyophilized agent, a crystalline solid, a salt in crystalline form, crystalline sodium chloride, crystalline sugar, a solid dialysate precursor, a saturated solution, an unsaturated solution, an at least partially liquid concentrate, and an at least partially solid concentrate. In some embodiments, each of the medical bags may include an interior volume divided into multiple internal compartments by at least one frangible partition. In some embodiments, each of the medical bags may include an interior volume with at least one first compartment which is devoid of contents and at least one second compartment at least partially filled with a concentrate. In some embodiments, the chain may include a weakened section between each of the medical bags. In some embodiments, the chain may comprise at least 50 medical bags. In some embodiments, the dispenser may be no more than 10 kg. In some embodiments, a first side of the housing may include a first and second face. The first face may be substantially parallel to an opposing side of the housing. The second face may extend from an edge of the first face at an angle toward the opposing side of the housing. The outlet may be defined in the second face. In some embodiments, the reel may be coupled to a tensioner assembly. In some embodiments, the dispenser may be devoid of metallic components. In some embodiments, the cover assembly may include a sealing member. The sealing member may be held in compression against the housing by a lock assembly. In some embodiments, the dispenser may further comprise a plurality of bolt retainers surrounding the outlet and a sealing member disposed about the outlet. In some embodiments, the cover assembly may include a sealing body having an exterior face. The sealing body may have a sealing surface along the periphery of the exterior face.

In accordance with another example embodiment of the present disclosure, an example method of packaging fluid into a medical bag may comprise positioning the medical bag in a first position in which a septum in a port of the bag is aligned with a fluid dispensing sharp. The method may further comprise irradiating at least an exposed surface of the septum with an irradiation assembly. The method may further comprise positioning the medical bag in a second position in which the fluid dispensing sharp has pierced through the septum and is in fluid communication with an empty compartment within an interior volume of medical bag. The method may further comprise dispensing a predetermined amount of diluent from the fluid dispensing sharp into the medical bag. The method may further comprise mixing diluent with a concentrate in the medical bag by compromising a temporary partition separating the empty compartment from the concentrate while the predetermined amount of diluent is transferred into the interior volume and dropping the diluent into the concentrate.

In some embodiments, the method may further comprise further mixing the diluent with the concentrate by compromising a second temporary partition in the interior volume while the predetermined amount of diluent is transferred into the medical bag and dropping the diluent and concentrate to an end of the bag opposite the port. In some embodiments, the method may further comprise dissolving the concentrate. In some embodiments, the method may further comprise displacing the bag to a mix assisting assembly and agitating the diluent and concentrate within the interior volume. In some embodiments, positioning the medical bag in the first position may comprise positioning the port at least partially into an airflow channel surrounding the fluid dispensing sharp. In some embodiments, the method may further comprise flowing air through the airflow channel. In some embodiments, the method may further comprise providing a laminar flow of clean air to the airflow channel. In some embodiments, irradiating at least the exposed surface of the septum may comprise illuminating the exposed surface of the septum with UV light. In some embodiments, irradiating at least the exposed surface of the septum may comprise powering an array of UVC LEDs. In some embodiments, irradiating at least the exposed surface of the septum may comprise powering at least one antimicrobial light emitter. In some embodiments, positioning the medical bag in the first position may comprise displacing the port within an irradiation transmissive barrier. In some embodiments, irradiating at least the exposed surface of the septum may comprise also irradiating at least a portion of the fluid dispensing sharp. In some embodiments, irradiating at least the exposed surface of the septum may comprise irradiating the exposed surface of the septum with at least a prescribed dosage of irradiation. In some embodiments, dispensing the predetermined amount of diluent from the fluid dispensing sharp may comprise dispensing the predetermined amount of diluent from at least one side port in a pencil tip needle. In some embodiments, the concentrate may be a solid concentrate. In some embodiments, the solid concentrate may be a concentrate for a solution selected from a list consisting of a saline solution, a sugar solution, a saline and sugar solution, normal saline, half normal saline, D5W, peritoneal dialysate, hemodialysis dialysate, Ringer's solution, lactated Ringer's solution, Hartmann's Solution, hypotonic solution, isotonic solution, and hypertonic solution. In some embodiments, the solid concentrate may be selected from a list consisting of, powder, lyophilized medical agent, crystalline concentrate, salt concentrate, sodium chloride concentrate, sugar concentrate, dextrose concentrate. In some embodiments, positioning the bag in at least one of the first position and second position may comprise directing the port with at least one guide.

In accordance with another example embodiment of the present disclosure, an example method of packaging fluid into a medical bag may comprise positioning the medical bag in a first position in which a septum in a port of the bag is aligned with a fluid dispensing sharp. The method may further comprise illuminating at least an exposed portion of the septum with at least one antimicrobial light emitter. The method may further comprise positioning the medical bag in a second position in which the fluid dispensing sharp has pierced through the septum and is in fluid communication with an empty compartment within an interior volume of medical bag. The method may further comprise dispensing a predetermined amount of fluid from the fluid dispensing sharp into the medical bag. The method may further comprise retaining the medical bag in a mix assisting assembly and rotating the medical bag in alternating clockwise and counterclockwise directions about an axis substantially perpendicular to a plane of the medical bag in which the axis of the port falls through a sequence of predetermined positions.

In some embodiments, the medical bag may be rotated at at least a certain rotation rate as the medical bag is rotated through the sequence of predetermined positions. In some embodiments, the method may further comprise dwelling at each of the predetermined positions. In some embodiments, rotating the medical bag may comprise fully inverting the medical bag at least once. In some embodiments, retaining the medical bag in the mix assisting assembly may comprise placing the medical bag in a cradle of the mix assisting assembly. In some embodiments, the method further comprises mixing the fluid with a concentrate in the medical bag by compromising a temporary partition separating and empty compartment with the medical bag from the concentrate while the predetermined amount of fluid is transferred into the interior volume and dropping the fluid into the concentrate. In some embodiments, the method may further comprise further mixing the fluid with the concentrate by compromising a second temporary partition in the interior volume while the predetermined amount of fluid is transferred into the medical bag and dropping the fluid and concentrate to an end of the bag opposite the port. In some embodiments, rotating the medical bag may further comprise dissolving a concentrate within the medical bag into the predetermined amount of fluid. In some embodiments, positioning the medical bag in the first position may comprise positioning the port at least partially into an airflow channel surrounding the fluid dispensing sharp. In some embodiments, the method may further comprise flowing air through the airflow channel. In some embodiments, the method may further comprise providing a laminar flow of clean air to the airflow channel. In some embodiments, the at least one antimicrobial light emitter may include a UVC LED. In some embodiments, positioning the medical bag in the first position may comprise displacing the port within an irradiation transmissive barrier of a fluid dispensing cartridge. In some embodiments, the method may further comprise illuminating at least a portion of the fluid dispensing sharp with the at least one antimicrobial light emitter. In some embodiments, the method may further comprise mixing the predetermined amount of fluid with a concentrate within the medical bag for a solution selected from a list consisting of a saline solution, a sugar solution, a saline and sugar solution, normal saline, half normal saline, D5W, peritoneal dialysate, hemodialysis dialysate, Ringer's solution, lactated Ringer's solution, Hartmann's Solution, hypotonic solution, isotonic solution, and hypertonic solution. In some embodiments, the method may further comprise mixing the predetermined amount of fluid with a concentrate within the medical bas selected from a list consisting of, powder, lyophilized medical agent, crystalline concentrate, salt concentrate, sodium chloride concentrate, sugar concentrate, and dextrose concentrate. In some embodiments, positioning the medical bag in at least one of the first position and second position may comprise directing the port with at least one guide.

In accordance with another example embodiment of the present disclosure a multi-compartment flexible reservoir for medical agent may comprise a first sheet and second sheet of material sealed to one another along a peripheral seal. The peripheral seal may define an interior volume of the reservoir. The reservoir may further comprise a plurality for ports coupled into the peripheral seal. Each of the ports may define an interior flow path in communication with the interior volume. The reservoir may further comprise a first compartment of the interior volume in fluid communication with each of the plurality of ports. The first compartment may be collapsed and devoid of content. The reservoir may further comprise a second compartment of the interior volume defined by a first and second first frangible seal each extending across the reservoir at respective opposing ends of the second compartment. The first and second sheet may be coupled together to form each of the first and second frangible seal. The second compartment may be at least partially filled with a concentrate and separated from the first compartment by the first frangible seal. The reservoir may further comprise a third compartment at an end of the reservoir opposite the ports. The third compartment may be segregated from the second compartment by the second frangible seal. The third compartment may be collapsed and devoid of content.

In some embodiments, at least one of the plurality of ports may include a septum disposed in an end of the port most distal the interior volume. In some embodiments, at least one of the plurality of ports may include a stopper with a spike receptacle. The stopper may be disposed in an end of the port most distal the interior volume. In some embodiments, the plurality of ports may include a first port and a second port. In some embodiments, at least one of the plurality of ports may include a set or projections extending outwardly from an axis of the port. In some embodiments, the set of projections may include a set of ribs. In some embodiments, the set of projections may include in at least one projection bearing body coupled to the port. In some embodiments, at least one of the plurality of ports may include at least one outwardly extending projection which at least partially surrounds the port. In some embodiments, the second compartment may be filled with a solid concentrate. In some embodiments, the solid concentrate may be a concentrate for a solution selected from a list consisting of a saline solution, a sugar solution, a saline and sugar solution, normal saline, half normal saline, D5W, peritoneal dialysate, hemodialysis dialysate, Ringer's solution, lactated Ringer's solution, Hartmann's Solution, hypotonic solution, isotonic solution, and hypertonic solution. In some embodiments, the solid concentrate may be selected from a list consisting of, powder, lyophilized medical agent, crystalline concentrate, salt concentrate, sodium chloride concentrate, sugar concentrate, dextrose concentrate. In some embodiments, the second compartment may be closer to the ports than to an end of the reservoir opposite the ports. In some embodiments, the first frangible seal may extend across the reservoir in a direction substantially parallel to a portion of the peripheral seal into which the ports are coupled. The first frangible seal may be disposed 5-25% of the length of the reservoir from the peripheral seal into which the ports are coupled. In some embodiments, a capacity of the second compartment may be greater than a volume of the concentrate disposed in the second compartment.

In accordance with another example embodiment of the present disclosure, a multi-compartment reservoir for medical agent may comprise a first and second sheet of material sealed to one another at a peripheral seal to define an interior volume of the reservoir. The reservoir may further comprise a first port and a second port bonded into the peripheral seal. Each of the first port and second may define an interior flow path in communication with the interior volume. Each of the first port and second port may have a set of projections extending therefrom and at least partially surrounding the respective port. The reservoir may further comprise a first compartment in fluid communication with the ports. The first compartment may be collapsed and devoid of contents. The reservoir may further comprise a second compartment containing a concentrate. The reservoir may further comprise a frangible partition separating the first and second compartment. The first and second sheet may be coupled together to form the frangible partition.

In some embodiments, the frangible partition may extend across the reservoir in a direction substantially parallel to a portion of the peripheral seal into which the ports are coupled. The frangible partition may be disposed 5-25% of the length of the reservoir from the peripheral seal into which the ports are coupled. In some embodiments, a capacity of the second compartment may be greater than a volume of the concentrate disposed in the second compartment. In some embodiments, the second compartment may be filled with a solid concentrate. In some embodiments, the solid concentrate may be a concentrate for a solution selected from a list consisting of a saline solution, a sugar solution, a saline and sugar solution, normal saline, half normal saline, D5W, peritoneal dialysate, hemodialysis dialysate, Ringer's solution, lactated Ringer's solution, Hartmann's Solution, hypotonic solution, isotonic solution, and hypertonic solution. In some embodiments, the solid concentrate may be selected from a list consisting of, powder, lyophilized medical agent, crystalline concentrate, salt concentrate, sodium chloride concentrate, sugar concentrate, dextrose concentrate. In some embodiments, the projections in each set of projections may be ribs. In some embodiments, the set of projections extending from each port may include at least one first projection and at least one second projection. The at least one second projection may be offset from the at least one first projection along an axial dimension of the port. In some embodiments, the first port may include a septum disposed in an end of the port most distal the interior volume. In some embodiments, the second port may include a stopper with a spike receptacle, the stopper disposed in an end of the port most distal the interior volume. In some embodiments, the reservoir may further comprise a third compartment at an end of the reservoir opposite the first and second port. The third compartment may be segregated from the second compartment by another frangible partition. The third interior compartment may be collapsed and devoid of content.

In accordance with an embodiment of the present disclosure, a multi-compartment reservoir for medical agent may comprise a first and second sheet of material sealed to one another at a peripheral seal to define an interior volume of the bag. The reservoir may further comprise a first port and a second port coupled into the peripheral seal. Each of the first port and second port may define an interior flow path in communication with the interior volume. Each of the first port and second port may have a set of projections extending therefrom and at least partially surrounding the respective port. The reservoir may further comprise a first compartment in fluid communication with the ports. The first compartment may be collapsed and devoid of contents. The reservoir may further comprise a second compartment containing a concentrate. The reservoir may further comprise a frangible partition separating the first and second compartment. The first and second sheet may be coupled together to form the frangible partition.

In some embodiments, a capacity of the second compartment may be greater than a volume of the concentrate disposed in the second compartment. In some embodiments, the second compartment may be filled with an at least partially liquid concentrate. In some embodiments, the at least partially liquid concentrate may be selected from a list consisting of saline brine, sodium chloride brine, sugar solution, dialysate concentrate, sugar and saline solution, Ringer's solution concentrate, lactated Ringer's solution concentrate, Hartmann's solution concentrate, saturated solution, supersaturated solution, and unsaturated solution. In some embodiments, the second compartment may be filled with a solid concentrate. In some embodiments, the solid concentrate may be a concentrate for a solution selected from a list consisting of a saline solution, a sugar solution, a saline and sugar solution, normal saline, half normal saline, D5W, peritoneal dialysate, hemodialysis dialysate, Ringer's solution, lactated Ringer's solution, Hartmann's Solution, hypotonic solution, isotonic solution, and hypertonic solution. In some embodiments, the solid concentrate may be selected from a list consisting of, powder, lyophilized medical agent, crystalline concentrate, salt concentrate, sodium chloride concentrate, sugar concentrate, dextrose concentrate. In some embodiments, the projections in each set of projections may be ribs. In some embodiments, the set of projections extending from each port may include at least one first projection and at least one second projection. The at least one second projection may be offset from the at least one first projection along an axial dimension of the port. In some embodiments, the set of projections extending from the first port may be monolithically formed with the first port and the second set of projections extending from the second port are monolithically formed with the second port. In some embodiments, the set of projections on each of the first port and the second port may be included on a ribbing clip coupled to the first and second port. In some embodiments, the set of projections on each of the first port and the second port may be included on sets of ribbed rings coupled to each of the first and second ports. In some embodiments, the first and second port may be formed of a rigid plastic. In some embodiments, the first port may include a septum disposed in an end of the port most distal the interior volume. In some embodiments, the second port may include a stopper with a spike receptacle, the stopper disposed in an end of the port most distal the interior volume. In some embodiments, the reservoir may further comprise a third compartment at an end of the reservoir opposite the first and second port. The third compartment may be segregated from the second compartment by another frangible partition. The third compartment may be collapsed and devoid of content.

In accordance with another example embodiment of the present disclosure, an example concentrate containing reservoir for medical agent may comprise a first and second sheet of material sealed to one another at a peripheral seal to define an interior volume of the bag. The first and second sheet of material may be permeable to a solvent. The reservoir may further comprise a first port and a second port coupled into the peripheral seal. Each of the first port and second port may define an interior flow path in communication with the interior volume. The reservoir may further comprise an at least partially solid concentrate within the interior volume. The at least partially solid concentrate may be particulate free and formed within the interior volume from a filtered liquid concentrate, at least one solute, and the solvent.

In some embodiments, each of the first port and second port may have a set of projections extending therefrom and at least partially surrounding the respective port. In some embodiments, the projections in each set of projections may be ribs. In some embodiments, the set of projections extending from each port may include at least one first projection and at least one second projection. In some embodiments, the at least one second projection may be offset from the at least one first projection along an axial dimension of the port. In some embodiments, the set of projections extending from the first port may be monolithically formed with the first port and the second set of projections extending from the second port are monolithically formed with the second port. In some embodiments, the set of projections on each of the first port and the second port may be included on one of a list consisting of a ribbing clip coupled to the first and second port, a set of projection bearing bodies each coupled to a respective one of the first and second port, a spacer clip coupled to the first and second port, and sets of ribbed rings coupled to each of the first and second ports. In some embodiments, the first and second port may be formed of a rigid plastic. In some embodiments, the at least partially solid concentrate may include a saturated solution and one or more crystallized bodies formed of a constituent of the concentrate. In some embodiments, the at least partially solid concentrate may be a number of crystallized bodies. In some embodiments, the at least partially solid concentrate may be concentrate for a solution selected from a list consisting of a saline solution, a sugar solution, a saline and sugar solution, normal saline, half normal saline, D5W, peritoneal dialysate, hemodialysis dialysate, Ringer's solution, lactated Ringer's solution, Hartmann's Solution, hypotonic solution, isotonic solution, and hypertonic solution. In some embodiments, the at least partially solid concentrate may include a liquid selected from a list consisting of saline brine, sodium chloride brine, sugar solution, dialysate concentrate, sugar and saline solution, Ringer's solution concentrate, lactated Ringer's solution concentrate, and Hartmann's solution concentrate. In some embodiments, the interior volume of the reservoir may include a frangible partition separating a first compartment in fluid communication with the ports from a second compartment. The first and second sheet may be coupled together to form the frangible partition. The at least partially solid concentrate being may be included in the second compartment. In some embodiments, the first compartment may be devoid of contents. In some embodiments, the reservoir may further comprise a third compartment at an end of the reservoir opposite the first and second port. The third compartment may be segregated from the second compartment by another frangible partition. The third compartment may be collapsed and devoid of contents.

In accordance with another example embodiment of the present disclosure a method of producing a medical reservoir containing an at least partially solid concentrate may comprise transferring a precursor solution through at least one filter and into an interior volume of the medical reservoir. The method may further comprise subjecting the medical reservoir to a controlled environment. The method may further comprise evaporating at least a portion of a solvent component from the precursor solution out of the medical reservoir through the material forming the medical reservoir. The method may further comprise at least partially recrystallizing at least one solute component of the precursor solution within the medical reservoir.

In some embodiments, the controlled environment may be temperature controlled. In some embodiments, the controlled environment may be humidity controlled. In some embodiments, subjecting the medical reservoir to a controlled environment may comprise maintaining the ambient temperature in the controlled environment at at least 50° C. In some embodiments, subjecting the medical reservoir to a controlled environment may comprise maintaining the ambient humidity in the controlled environment at no more than 15%. In some embodiments, the precursor solution may be a saturated solution. In some embodiments, the method may further comprise heating a solvent to a temperature set point and adding at least one solute to the solvent to create the precursor solution such that the precursor solution is a saturated solution at the temperature set point. In some embodiments, evaporating the solvent component from the precursor solution may comprise evaporating water from the precursor solution. In some embodiments, the precursor solution may be selected from a list consisting of a saline brine, a sodium chloride brine, a sugar solution, a saline and sugar solution, a dialysate precursor, a Ringer's solution concentrate, lactated Ringer's solution concentrate, and Hartmann's solution concentrate. In some embodiments, the method may further comprise terminally sterilizing the medical reservoir. In some embodiments, the method may further comprise placing a septum in a port of the medical reservoir. In some embodiments, subjecting the medical reservoir to the controlled environment may comprise orienting the medical reservoir such that at least one port of the medical reservoir is disposed at a top of the medical reservoir. In some embodiments, the method may further comprise isolating the precursor solution within a compartment of the interior volume by forming a frangible partition within the interior volume.

In accordance with another example embodiment of the present disclosure a method of producing a chain of medical reservoirs each containing an at least partially solid concentrate may comprise transferring a precursor solution through at least one filter and into an interior volume of each of the medical reservoirs. The method may further comprise subjecting the chain of medical reservoirs to a controlled environment. The method may further comprise evaporating at least a portion of a solvent component from the precursor solution out of each of the medical reservoirs through the material forming the respective medical reservoirs. The method may further comprise at least partially recrystallizing at least one solute component of the precursor solution within the each of the medical reservoirs.

In some embodiments, the controlled environment may be temperature controlled. In some embodiments, the controlled environment may be humidity controlled. In some embodiments, subjecting the medical reservoir chain to a controlled environment may comprise maintaining the ambient temperature in the controlled environment at at least 50° C. In some embodiments, subjecting the medical reservoir chain to a controlled environment may comprise maintaining the ambient humidity in the controlled environment at no more than 15%. In some embodiments, the precursor solution may be a saturated solution. In some embodiments, the method further comprises heating a solvent to a temperature set point and adding at least one solute to the solvent to create the precursor solution such that the precursor solution is a saturated solution at the temperature set point. In some embodiments, evaporating the solvent component from the precursor solution in the interior volume of each medical reservoir may comprise evaporating water. In some embodiments, the precursor solution may be selected from a list consisting of a saline brine, a sodium chloride brine, a sugar solution, a saline and sugar solution, a dialysate precursor, a Ringer's solution concentrate, lactated Ringer's solution concentrate, and Hartmann's solution concentrate. In some embodiments, the method may further comprise terminally sterilizing the chain of medical reservoirs. In some embodiments, the method may further comprise placing a septum in a port of the each of the medical reservoirs. In some embodiments, subjecting the chain of medical reservoirs to the controlled environment may comprise orienting the medical reservoirs such that at least one port of each of the medical reservoirs is disposed at a top of the respective medical reservoir. In some embodiments, the method may further comprise isolating the precursor solution within a compartment of the interior volume of each medical reservoir by forming a frangible partition within the interior volume of each medical reservoir.

In accordance with another example embodiment of the present disclosure a spool of medical reservoirs may comprise a lead medical reservoir. The spool may further comprise a terminal medical reservoir. The spool may further comprise a plurality of intermediate medical reservoirs coupled together in series. The spool may further comprise a tail. The spool may further comprise a spool body having a core and opposing end flanges. An end of the tail may be coupled to core. The lead medical reservoir may be coupled to a first of the plurality of intermediate reservoirs. The last of the plurality of intermediate reservoirs may be coupled to the terminal medical reservoir.

In some embodiments, each of the medical reservoirs may define an interior volume containing at least one concentrate. In some embodiments, each of the at least one concentrate may be selected from a list consisting of a saline brine, a sodium chloride brine, a sugar solution, a saline and sugar solution, a peritoneal dialysate precursor solution, a hemodialysis precursor solution, a Ringer's solution concentrate, lactated Ringer's solution concentrate, and Hartmann's solution concentrate, a powder, a lyophilized agent, a crystalline solid, a salt in crystalline form, crystalline sodium chloride, crystalline sugar, a solid dialysate precursor, a saturated solution, an unsaturated solution, an at least partially liquid concentrate, and an at least partially solid concentrate. In some embodiments, each of the medical reservoirs may define an interior volume separated into multiple internal compartments by at least one frangible partition. In some embodiments, each of the medical reservoirs may define an interior volume having a first compartment at least partially filled with a concentrate and at least one second compartment which is devoid of contents. In some embodiments, the tail may include at least one reservoir. In some embodiments, the lead reservoir, plurality of intermediate reservoirs, and terminal reservoir may be chained abreast of one another. In some embodiments, the lead reservoir, plurality of intermediate reservoirs, and terminal reservoir may be chained end to end. In some embodiments, the chain may include a coupling region between each of the medical reservoir. In some embodiments, the coupling region may include a weakened section. In some embodiments, each weakened section may include at least one score line. In some embodiments, each weakened section may include a series of perforations. In some embodiments, the coupling region may include a number of bridges. In some embodiments, a score line may be present between each of the medical reservoirs. In some embodiments, the chain may include at least 50 reservoirs. In some embodiments, each of the medical reservoirs may be a bag. In some embodiments, the spool of medical reservoirs may further comprise a tensioner assembly. The tensioner assembly may be engaged with an end flange of the spool body.

In accordance with another example embodiment of the present disclosure, a medical reservoir chain may comprise a lead medical reservoir. The chain may further comprise a terminal medical reservoir. The chain may further comprise a plurality of intermediate medical reservoir coupled in series abreast of one another. A first of the plurality of intermediate medical reservoirs may be coupled to the lead medical reservoir and a second of the plurality of intermediate reservoirs. A last of the plurality of intermediate reservoirs may be coupled to a penultimate of the intermediate reservoirs and the terminal medical reservoir. Each of the medical reservoirs may define an interior volume containing at least one concentrate.

In some embodiments, each of the medical reservoirs may be a bag. In some embodiments, each of the at least one concentrate may be selected from a list consisting of a saline brine, a sodium chloride brine, a sugar solution, a saline and sugar solution, a peritoneal dialysate precursor solution, a hemodialysis precursor solution, a Ringer's solution concentrate, lactated Ringer's solution concentrate, and Hartmann's solution concentrate, a powder, a lyophilized agent, a crystalline solid, a salt in crystalline form, crystalline sodium chloride, crystalline sugar, a solid dialysate precursor, a saturated solution, an unsaturated solution, an at least partially liquid concentrate, and an at least partially solid concentrate. In some embodiments, the interior volume of each of the medical reservoirs may be divided into multiple internal compartments by at least one frangible partition. In some embodiments, the interior volume of each of the medical reservoirs may include at least one first compartment which is devoid of contents and at least one second compartment at least partially filled with a concentrate. In some embodiments, the chain of medical reservoirs may include a coupling region between each of the medical reservoirs. In some embodiments, the chain of medical reservoirs may include a weakened section between each of the medical reservoirs. In some embodiments, the chain of medical reservoirs may include at least one score line between each of the medical reservoirs. In some embodiments, the chain of medical reservoirs may include a series of perforations between each of the medical reservoirs. In some embodiments, the chain of medical reservoirs may include at least 50 medical reservoirs.

In accordance with another aspect of the present disclosure an example bag vending assembly for storing and dispensing IV bags may comprise a main body including a central member and a pair of lateral members spaced from and flanking the central member. The central member and lateral members may extend from a crosspiece of the main body. The assembly may further comprise a comb body coupled to the central member and displaceable relative to the central member from a first position to a second position. The comb body may have a first end, opposing second end and first and second sides extending from the first end to the second end. The assembly may further comprise a bias member interposed between the main body and the comb. The bias member may exert a bias urging the comb body toward the first position. The assembly may further comprise a first set of evenly spaced flexures extending in the direction of the comb body from the each of the lateral members. The assembly may further comprise a second set of evenly spaced flexures extending from the first side of the comb body in the direction of a lateral member of the pair of lateral members most proximate the first side of the comb body. The assembly may further comprise a third set of evenly spaced flexures extending from a second side of the comb body in the direction of a lateral member of the pair of lateral members most proximate the second side of the comb body.

In some embodiments, the comb body may include at least one track and the central member may include at least one guide which extends into the track. In some embodiments, the central member may include at least one track and the comb body may include at least one guide which extends into the track. In some embodiments, the crosspiece may include a crosspiece wall and the second end of the comb body may define a stop surface. The stop surface may be in contact with the crosspiece wall when the comb body is in the second position. In some embodiments, the second set of evenly spaced flexures and the flexures of the first set of evenly spaced flexures of the lateral member of the pair of lateral members most proximate the first side of the comb body may be symmetric about a midplane between the central member and the lateral member of the pair of lateral members most proximate the first side of the comb body when the comb body is in the first position. In some embodiments, the third set of evenly spaced flexures and the flexures of the first set of evenly spaced flexures of the lateral member of the pair of lateral members most proximate the second side of the comb body may be symmetric about a midplane between the central member and the lateral member of the pair of lateral members most proximate the second side of the comb body when the comb body is in the first position. In some embodiments, each the first set of evenly spaced flexures may extend from each of the lateral members at an oblique angle. In some embodiments, each of the second set of evenly spaced flexure and each of the third set of evenly spaced flexures may extend from the comb body at an oblique angle.

In accordance with another example embodiment of the present disclose and example bag vending assembly for storing and dispensing IV bags may comprise a main body including a central member and a pair of lateral members spaced from and flanking the central member. The central member and lateral members may extend from a crosspiece of the main body. The assembly may further comprise a comb body coupled to the central member and displaceable relative to the central member from a first position to a second position. The comb body may have a first end and opposing second end. The comb body may include a first comb ratcheting member coupled to a first side of a main segment of the comb body by a first plurality of flexures and a second comb ratcheting member coupled to a second side of the main segment opposite the first side of the main segment by a second plurality of flexures. The assembly may further comprise a first lateral ratcheting member coupled to a lateral member of the pair of lateral members most proximate the first comb ratcheting member by a third plurality of flexures. The assembly may further comprise a second lateral ratcheting member coupled to a lateral member of the pair of lateral members most proximate the second comb ratcheting member by a fourth plurality of flexures.

In some embodiments, the main segment of the comb body may include at least one track and the central member may include at least one guide which extends into the track. In some embodiments, the central member may include at least one track and the main segment of the comb body may include at least one guide which extends into the track. In some embodiments, the crosspiece may include a crosspiece wall and the second end of the comb body may define a stop surface. The stop surface may be in contact with the crosspiece wall when the comb body is in the second position. In some embodiments, the first comb ratcheting member and the first lateral ratcheting member may be symmetric about a midplane between the central member and the lateral member of the pair of lateral members most proximate the first comb ratcheting member when the comb body is in the first position. In some embodiments, the second comb ratcheting member and the second lateral ratcheting member may be symmetric about a midplane between the central member and the lateral member of the pair of lateral members most proximate the second comb ratcheting member when the comb body is in the first position. In some embodiments, each the first comb ratcheting member, second comb ratcheting member, first lateral ratcheting member, and second lateral ratcheting member may include a series of detents. In some embodiments, each of the detents may include an upstream portion most proximate the crosspiece and a downstream portion more distal to the crosspiece with respect to the upstream portion. Each of the upstream portion and downstream portion may be sloped in opposing directions. The slope of the upstream portion may be sharper than the slope of the downstream portion. In some embodiments, a transition region may be present between each detent in the series of detents on each of the first comb ratcheting member, second comb ratcheting member, first lateral ratcheting member, and second lateral ratcheting member. The transition region may be a region where the downstream and upstream region of adjacent detents met. In some embodiments, at least three flexures may be included in each of the first, second, third, and fourth plurality of flexures. In some embodiments, each of the first, second, third, and fourth plurality of flexures may include an upstream flexure coupled to an end region of the respective the first comb ratcheting member, second comb ratcheting member, first lateral ratcheting member, and second lateral ratcheting member most proximate the crosspiece. Each plurality of flexures may include a downstream flexure coupled to an end of the respective the first comb ratcheting member, second comb ratcheting member, first lateral ratcheting member, and second lateral ratcheting member most distal the crosspiece. Each plurality of flexures may include at least one intermediate flexure intermediate the upstream flexure and downstream flexure. In some embodiments, the main body may include a flange including a number of lobes configured to accept mounting fasteners.

In accordance with another example embodiment of the present disclosure a bag vending assembly for storing and dispensing IV bags may comprise a main body including a central member and a first lateral member and a second lateral member each flanking the central member and separated from the central member by respective first and second channels. The central member and lateral members may extend from a crosspiece of the main body. The assembly may further comprise a first cantilevered finger projecting from the first lateral member into the first channel towards the central member. The first cantilevered finger may have a nub extending toward the central member from an unsupported end of the first cantilevered finger. The assembly may further comprise a second cantilevered finger projecting from the second lateral member into the second channel towards the central member. The second cantilevered finger may have a nub extending toward the central member from an unsupported end of the second cantilevered finger. The assembly may further comprise a follower including a bay. The follower may at least partially surround the central member and may be displaceable along the axis of the central member. The follower may include a first leg portion which extends into the first channel and a second leg portion which extends into the second channel. The assembly may further comprise a constant force spring including a mounting end coupled to an end region of the central member most distal to the crosspiece and a coil coupled to the mounting end. The coil may be at least partially disposed within the bay.

In some embodiments, the main body may include a flange including a number of lobes configured to accept mounting fasteners. In some embodiments, the central member may include a prong. The mounting end of the constant force spring may include an aperture. The prong may extend though the aperture to couple the constant force spring to the central member. In some embodiments, the mounting end of the constant force spring may be coupled to the end region of the central member via a fastener. In some embodiments, a span of the constant force spring between the mounting end and the coil may be routed intermediate central member and a face of the follower and at least partially within a trough defined in the face of the follower. In some embodiments, the unsupported end of the first cantilevered finger and the unsupported end of the second cantilevered finger may be even with the end of the central member most distal to the crosspiece. In some embodiments, the central projection may include a stop projection extending therefrom. The stop projection presenting a mechanical interference to displacement of the follower when the follower is in contact with the stop projection. In some embodiments, a projection may extend from the first leg portion toward the second leg portion and a second projection may extend from the second leg portion toward the first leg portion. The first and second projections may be disposed adjacent a first side of the central member. The portion of the follower including the bay may be disposed adjacent an opposing second side of the central member. In some embodiments, each channel may include a narrow portion and a wide portion. The wide portion of each channel may be disposed at the portion of each channel most proximate the crosspiece. In some embodiments, the first face of the main body flanking each side of the channels may be substantially planar.

In accordance with another example embodiment of the present disclosure, an example dispenser of medical bag reservoirs may comprise a housing having an outlet. The dispenser may further comprise a removable cover sealing the outlet. The dispenser may further comprise a bag vending assembly coupled within the housing. The bag vending assembly may have a vending output in alignment with the outlet. The dispenser may further comprise a plurality of individual medical bag reservoirs each having a set of ports. Each port may include a set projections extending from the exterior surface. The ports of each medical bag reservoir may be disposed in the bag vending assembly with the projections overhanging a portion of the bag vending assembly. The ports of a foremost medical bag reservoir of the plurality of individual medical bag reservoirs may be retained within the bag vending assembly by the vending output.

In some embodiments, each of the medical bag reservoirs may contain at least one concentrate selected from a list consisting of a saline brine, a sodium chloride brine, a sugar solution, a saline and sugar solution, a peritoneal dialysate precursor solution, a hemodialysis precursor solution, a Ringer's solution concentrate, lactated Ringer's solution concentrate, and Hartmann's solution concentrate, a powder, a lyophilized agent, a crystalline solid, a salt in crystalline form, crystalline sodium chloride, crystalline sugar, a solid dialysate precursor, a saturated solution, an unsaturated solution, an at least partially liquid concentrate, and an at least partially solid concentrate. In some embodiments, each of the medical bag reservoirs may include an interior volume divided into multiple internal compartments by at least one frangible partition. In some embodiments, wherein each of the medical bag reservoirs may include an interior volume with at least one first compartment which is devoid of contents and at least one second compartment at least partially filled with a concentrate. In some embodiments, the housing may be at least partially formed of a flaccid, flexible material. In some embodiments, the dispenser may be devoid of metallic components. In some embodiments, the cover assembly may include a sealing member. The sealing member may be held in compression against the housing by a lock assembly and the dispenser may further comprise a plurality of bolt retainers surrounding the outlet. In some embodiments, the bag vending assembly may include a follower disposed behind a rearmost medical bag reservoir opposite the foremost medical bag reservoir. The follower may have a displacement range along a guide projection. The follower may be biased toward the vending outlet by a constant force spring. The ports may be disposed at least partially in channels on either side of the guide projection. The follower may include legs which extend into the channels. In some embodiments, the bag vending assembly may include a stationary portion and a displaceable comb portion. The comb portion may include a first and second side each including a series of flexures. The stationary portion may include complimentary flexures extending toward each of the first and second side of the comb portion. The ports of the individual medical bag reservoirs may be held between the series of flexures and complimentary flexures. In some embodiments, the bag vending assembly may include a stationary portion and a displaceable comb portion. The comb portion may include a main segment with a first side and a second side each having a comb ratcheting member coupled thereto by a plurality of flexures. The stationary portion may include complimentary ratcheting members extending toward each of the first and second side of main segment of the comb portion. The ports of the individual medical bag reservoirs may be held between detents defined in the comb ratcheting members and complimentary ratcheting members.

In accordance with another example embodiment of the present disclosure an example ribbed spacer for use as an accessory to an IV bag which maintains a prescribed spacing between a set of ports of the IV bag may comprise a first arced wall having a first arced wall first side surface and an opposing first arced wall second side surface. The first arced wall may have a first plurality of ribs extending outwardly from the first arced wall first side surface. The first arced wall may include a set of first fenestrations extending therethrough. There may be a ledge surface defined by a sidewall of each of the set of first fenestrations. The first arced wall may have a set of first catch projections equal in number to the number of fenestrations in the set of first fenestrations. The first catch projections may extend perpendicular to a long axis of the first arced wall and each may include a first projection catch protuberance at an unsupported end thereof. The ribbed spacer may further comprise a second arced wall having a second arced wall first side surface and an opposing second arced wall second side surface. The second arced wall may have a second plurality of ribs extending outwardly from the second arced wall first side surface. The second arced wall may include a set of second fenestrations extending therethrough. There may be a ledge surface defined by a sidewall of each of the set of second fenestrations. The second arced wall may have a set of second catch projections equal in number to the number of fenestrations in the set of second fenestrations. The second catch projections may extend perpendicular to a long axis of the second arced wall and may each include a second projection catch protuberance at an unsupported end thereof. The ribbed spacer may further comprise a spacer bridge coupling the first and second arced wall.

In some embodiments, the spacer bridge may be formed monolithically with the first arced wall and second arced wall. In some embodiments, the first catch projections may extend from the first arced wall at an edge of the first arced wall most distal to the spacer bridge and the second catch projections may extend from the second arced wall at an edge of the second arced wall most proximate the spacer bridge. In some embodiments, the first arced wall may include a first arced wall first half most proximate the spacer bridge and a first arced wall second half more distal the spacer bridge than the first half. The first fenestrations may be disposed in the first arced wall first half. The second arced wall may include a second arced wall first half most proximate the spacer bridge and a second arced wall second half more distal the spacer bridge than the first half. The second fenestrations may be disposed in the second arced wall second half. In some embodiments, the first arced wall second surface and second arced wall second surface may be semi-circular. In some embodiments, each of the first and second arced wall may include a first end and an opposing second end. The spacer bridge may extend between the first and second arced walls at the first ends of the first and second arced walls. In some embodiments, each of the first and second arced wall may include a first end and an opposing second end. A set of second end ribs of the first plurality of ribs may extend from the first arced wall at the second end of the first arced wall. A set of second end ribs of the second plurality of ribs may extend from the second arced wall at the second end of the second arced wall. In some embodiments, a set of intermediary ribs of the first plurality of ribs may extend from the first arced wall intermediate the set of second end ribs of the first plurality of ribs and the first end of the first arced wall. A set of intermediary ribs of the second plurality of ribs may extend from the second arced wall intermediate the set of second end ribs of the second plurality of ribs and the first end of the second arced wall. In some embodiments, the first arced wall first surface may include a first flat face on the portion of first arced wall first surface most distal a plane along which the spacer bridge extends. The second arced wall first surface may include a second flat face on the portion of the second arced wall first surface most distal the plane along which the spacer bridge extends.

In accordance with another example embodiment of the present disclosure an example spacer clip for affixing to ports of a medical bag reservoir to maintain the spacing of the ports may comprise a pair of ribbed spacers. Each ribbed spacer may include a first ribbed sleeve and second ribbed sleeve coupled together by a spacer bridge. Each of the first and second ribbed sleeve may have an arced interior surface. Each of the first and second ribbed sleeve may include a set of apertures extending therethrough. There may be a ledge surface defined by a wall of each of the set of apertures. Each ribbed sleeve may include a set of catch projections equal in number to the number of apertures in the set of apertures. The catch projections may extend perpendicular to an axial dimension of the respective ribbed sleeve and may include a catch protuberance at an unsupported end thereof. A set of catch projections of the ribbed sleeves of a first of the pair of ribbed spacers may extend through respective apertures of the set of apertures of the ribbed sleeves of a second of the pair of ribbed spacers such that the catch protuberances of the catch projections of the ribbed sleeves of the first of the pair of ribbed spacers may be engaged with respective ledge surfaces of the ribbed sleeves of the second of the pair of ribbed spacers. The set of catch projections of the ribbed sleeves of the second of the pair of ribbed spacers may extend through respective apertures of the set of apertures of the ribbed sleeves of the first of the pair of ribbed spacers such that the catch protuberances of the catch projections of the ribbed sleeves of the second of the pair of ribbed spacers may be engaged with respective ledge surfaces of the ribbed sleeves of the first of the pair of ribbed spacers. The arced interior surfaces of the ribbed sleeves of the first and second ribbed spacers may cooperate to form bores each sized to accept a respective port of the ports.

In accordance with another example embodiment of the present disclosure, an example dispenser of medical bag reservoirs may comprise a rigid frame portion defining a central fenestration. The dispenser may further comprise a shell coupled to the frame portion. The dispenser may further comprise a removable cover assembly retained within the central fenestration. The removable cover assembly may include a panel with a port and a breathable microbial barrier extending across the port coupled to the panel. The frame portion, shell, and removable cover assembly may define an interior volume of the dispenser. The dispenser may further comprise a frangible coupled to and at least partially covering an exterior face of the frame portion and an exterior face of the panel. The frangible may have an aperture aligned with the port. The frangible may be coupled to the exterior face of the frame portion and the exterior face of the panel to form a seal around the port, a seal along the exterior face of the frame portion adjacent an edge the frame portion defining the central fenestration, and a seal along the exterior face of the panel adjacent the peripheral edge of the exterior face of the panel.

In some embodiments, the dispenser may further comprise a bag vending assembly coupled to the frame portion and within the interior volume of the dispenser. The bag vending assembly may have a vending output in alignment with the central fenestration. In some embodiments, the dispenser may further comprise a plurality of individual medical bag reservoirs each having a set of ports. Each port may include a set projections extending from the exterior surface. The ports of each medical bag reservoir may be disposed in the bag vending assembly with the projections overhanging a portion of the bag vending assembly. The ports of a foremost medical bag reservoir of the plurality of individual medical bag reservoirs may be retained within the bag vending assembly by the vending output. In some embodiments, each of the medical bag reservoirs may contain at least one concentrate selected from a list consisting of a saline brine, a sodium chloride brine, a sugar solution, a saline and sugar solution, a peritoneal dialysate precursor solution, a hemodialysis precursor solution, a Ringer's solution concentrate, lactated Ringer's solution concentrate, and Hartmann's solution concentrate, a powder, a lyophilized agent, a crystalline solid, a salt in crystalline form, crystalline sodium chloride, crystalline sugar, a solid dialysate precursor, a saturated solution, an unsaturated solution, an at least partially liquid concentrate, and an at least partially solid concentrate. In some embodiments, each of the medical bag reservoirs may include an interior volume divided into multiple internal compartments by at least one frangible partition. In some embodiments, each of the medical bag reservoirs may include an interior volume with at least one first compartment which is devoid of contents and at least one second compartment at least partially filled with a concentrate. In some embodiments, the bag vending assembly may include a follower disposed behind a rearmost medical bag reservoir opposite the foremost medical bag reservoir. The follower may have a displacement range along a guide projection. The follower may be biased toward the vending outlet by a constant force spring. The ports may be disposed at least partially in channels on either side of the guide projection. The follower may include legs which extend into the channels. In some embodiments, the bag vending assembly may include a stationary portion and a displaceable comb portion, the comb portion including a first and second side each including a series of flexures. The stationary portion may include complimentary flexures extending toward each of the first and second side of the comb portion. The ports of the individual medical bag reservoirs may be held between the series of flexures and complimentary flexures. In some embodiments, the bag vending assembly may include a stationary portion and a displaceable comb portion. The comb portion including a main segment with a first side and a second side each having a comb ratcheting member coupled thereto by a plurality of flexures. The stationary portion including complimentary ratcheting members extending toward each of the first and second side of main segment of the comb portion. The ports of the individual medical bag reservoirs being held between detents defined in the comb ratcheting members and complimentary ratcheting members. In some embodiments, the shell may be at least partially formed of a flaccid, flexible material. In some embodiments, the shell may be at least partially formed of a flexible, breathable microbial barrier material. In some embodiments, the dispenser may be devoid of metallic components. In some embodiments, the removable cover assembly may include a spring finger along at least one set of opposing peripheral edges of the removable cover assembly. Each spring finger of the set of spring fingers may be in a deflected state and in contact with a respective sidewall of the frame portion which defines the central fenestration. In some embodiments, the exterior face of the panel may include at least one set of guide recesses. The frangible may include recess apertures aligned with the guide recesses. The frangible may form a seal around each of the set of guide recesses. In some embodiments, the exterior face of the panel may include at least one depression. The frangible may include depression apertures aligned with the depression. The frangible may form a seal around each of the at least one depression. In some embodiments, a gap may be present between the edges of the frame portion adjacent the central fenestration and a perimeter of the exterior face of the panel. The frangible extending over the gap. In some embodiments, the dispenser may further comprise an outer housing surrounding the shell. The outer housing may be formed of a material with a rigidity greater than that of the shell. In some embodiments, the outer housing may be constructed of corrugated plastic plate sheet material. In some embodiments, the dispenser may further comprise an outer housing surrounding the shell so as to form a housing volume containing the shell. The shell may be coupled to the frame portion in an airtight manner. The outer housing may permit air communication from the housing volume to ambient. In some embodiments, the frangible may be a polymer sheet.

In accordance with another example embodiment of the present disclosure an example plug assembly for sealing an infeed access to an environmentally controlled enclosure may comprise a main plug portion including an exposed face with a central recessed region surrounded by a raised plateau region. The main plug portion may include a second face opposite the exposed face and sidewalls extending from the exposed face to the second face. The plug assembly may further comprise an exposed face compliant member surrounding the central recessed region and coupled to the raised plateau region adjacent a peripheral edge of the exposed face. The plug assembly may further comprise a cutter surrounding the central recessed region and coupled to the raised plateau region inbound of compliant member. The plug assembly may further comprise a sidewall compliant member coupled to the sidewall at a variable cross-section region of the main plug portion where the cross-sectional area of the main plug portion increases as distance to the second face decreases. The plug assembly may further comprise a negative pressure source in selective fluid communication with at least one vacuum port in a wall of the central recessed region via at least one vacuum port valve. The plug assembly may further comprise at least one plug assembly actuator coupled to the main plug portion. The main plug portion may be displaceable between a deployed position and a stowed position via the at least one plug assembly actuator.

In some embodiments, at least a portion of the raised plateau portion may be formed by a faceplate coupled to a base portion of the main plug portion. In some embodiments, the faceplate and portion of the sidewalls defined in the base portion may cooperate to define a dovetail shaped recess around the periphery of the main plug portion. The sidewall compliant member may be disposed at least partially within the dovetail shaped recess. In some embodiments, the plug assembly may further comprise at least one set of guidance pins extending from the central recessed region. In some embodiments, each guidance pin may include a tip. The tip may be disposed a greater distance from a plane even with the raised plateau region than the portion of the cutter most distal the plane even with the raised plateau region. In some embodiments, the cutter may include serrations. In some embodiments, the cutter may be a metallic rule die cutter. In some embodiments, at least one linear actuator may be coupled to the main plug portion within the central recessed region. Each of the at least one linear actuator may include a plunger displaceable from a retracted position to an extended position. In some embodiments, the at least one plug assembly actuator includes at least one rotary actuator and at least one linear actuator. In some embodiments, the plug assembly may further comprise a sealing assembly coupled to the main plug portion within the central recessed region. The sealing assembly may include a body displaceable along at least one guide between a depressed position and a protruding position. The plug assembly may include a bias member urging the body toward the protruding position. The may be a sealing member coupled to an outward facing surface of the body. In some embodiments, the body may include a pressure port extending through the body. The pressure port may be surrounded by the sealing member and in selective communication with a positive pressure source via at least positive pressure supply valve. In some embodiments, the plug assembly may further comprise at least one pressure sensor in fluid communication with the pressure port. In some embodiments, the plug assembly may further comprise at least one pressure sensor in fluid communication with at least one of the at least one vacuum port.

In accordance with another example embodiment of the present disclosure an example method of accessing an interior volume of a dispenser from an interior volume of an environmentally controlled enclosure may comprise displacing the dispenser toward a plug assembly sealed against an aperture in a wall of the enclosure. The dispenser may include a first side having a frame with a centrally disposed fenestration, a cover removably retained within the fenestration, and a frangible at least partially coupled to and at least partially overlaying the frame and cover. The method may further comprise forming a first seal between a first compliant member on an exterior face of the wall and a first side of the dispenser by pressing the frame against first compliant member. The first seal surrounding the aperture. The method may further comprise forming a second seal between a second compliant member on an exposed face of the plug assembly and the first side of the dispenser by pressing a peripheral region of the cover adjacent a perimeter edge of a exterior face of the cover against second compliant member, the second seal surrounding a recessed central region in the exposed face of the plug assembly. The method may further comprise compromising the frangible with a cutter of the plug assembly such that the frangible is separated into at least a first portion attached to the frame and a second portion attached to the cover. The method may further comprise establishing a vacuum in an intermediate volume between the cover and the recessed central region. The method may further comprise dislodging the cover from the dispenser by displacing the plug assembly toward a stowed position. The vacuum may hold the cover against the exposed face of the plug assembly as the plug assembly is displaced.

In some embodiments, displacing the dispenser toward the plug assembly may comprise powering at least one actuator of a docking assembly to displace at least one carriage of the docking assembly toward the wall. The dispenser may be coupled to the docking assembly. In some embodiments, compromising the frangible may comprise driving a serrated edge of the cutter through the frangible into a space between the frame and the perimeter edge of the exterior face of the cover. In some embodiments, dislodging the cover from the dispenser may comprise defeating a frictional engagement of spring fingers extending from at least two oppositely disposed sidewalls of the cover with the sidewall of the fenestration. In some embodiments, the method may further comprise displacing guide recesses in the cover onto guidance pins extending from the recessed central region. In some embodiments, the method may further comprise forming a seal between a face of a spring loaded body coupled to the recessed central region and the first side of the dispenser. The seal may surround a pressure port in the spring loaded body and surrounding a breathable microbial barrier extending across a port of the cover. In some embodiments, the method may further comprise placing the pressure port in fluid communication with a positive pressure source. In some embodiments, the method may further comprise isolating the pressure port from the positive pressure source and may further comprise confirming, with a controller, data output from a pressure sensor in fluid communication with the pressure port is indicative that at least one predefined pressure decay criterion is met. In some embodiments, the method may further comprise isolating the intermediate volume from a negative pressure source and may further comprise confirming, with a controller, data output from a pressure sensor in fluid communication with the intermediate volume is indicative that at least one predefined vacuum decay criterion is met. In some embodiments, the method may further comprise displacing the plug assembly from the stowed position to a deployed position in which it is sealed against the aperture in the wall of the enclosure and replacing the cover onto the dispenser. In some embodiments, replacing the cover onto the dispenser may comprise terminating the vacuum established in the intermediary volume. In some embodiments, replacing the cover onto the dispenser may comprise displacing at least one ejector body from a retracted position against the cover and in an ejecting direction to an ejecting position. The cover may displace in the ejecting direction as the ejector body is displaced to the ejecting position. In some embodiments, replacing the cover onto the dispenser may further comprise displacing a set of spring fingers on at least two oppositely disposed sidewalls of the cover past an interior face of the frame. The sidewalls of the frame may deflect the spring fingers as the spring fingers are displacing toward the interior face of the frame and restore to a resting state when past the interior face of the frame. In some embodiments, the method may further comprise transferring at least one reservoir from the interior volume of the dispenser into the interior volume of the environmentally controlled enclosure.

In accordance with another example embodiment of the present disclosure an example fluid dispensing assembly for a medical reservoir filling system may comprise a base plate including a first portion and a second portion. The second portion may have a cross-sectional area less than that of the first portion. The fluid dispensing assembly may further comprise an environmental seal member coupled to a sidewall of the second portion. The fluid dispensing assembly may further comprise a housing coupled to an end face of the second portion. The fluid dispensing assembly may further comprise an end cap coupled to a first side of the housing. The fluid dispensing assembly may further comprise a fluid inlet body coupled to a second side of the housing opposite the first side. The fluid dispensing assembly may further comprise a clean air supply body coupled to the fluid inlet body. The fluid dispensing assembly may further comprise an irradiation transmissive barrier captured between the housing and the end cap. The fluid dispensing assembly may further comprise an airflow channel extending along an airflow channel axis through the housing, end cap, fluid inlet body, and clean air supply body. The irradiation transmissive barrier may define at least a portion of the airflow channel. The fluid dispensing assembly may further comprise a plurality of antimicrobial light emitters housed within the housing. The irradiation transmissive barrier may be intermediate the plurality of antimicrobial light emitters and the airflow channel. The fluid dispensing assembly may further comprise a dispensing sharp coupled to a hub within the airflow channel. The hub may extend from the fluid inlet body. The dispensing sharp may be in fluid communication with a fluid source through the fluid inlet body.

In some embodiments, respective compliant bodies may be compressed between the housing and the end face of the second port, the end cap and the housing, the fluid inlet body and the housing, and the clean air supply body and the fluid inlet portion. In some embodiments, the end cap may include at least one port guide which projects into the airflow channel. In some embodiments, the dispensing sharp may be a pencil tipped sharp with a least one side port. In some embodiments, the airflow channel may vary in cross-sectional area along an axial dimension of at least the clean air supply body. In some embodiments, the irradiation transmissive barrier may be at least partially transparent to UVC light. In some embodiments, the irradiation transmissive barrier may be a tube of fused quartz. In some embodiments, at least one filter may be disposed intermediate the hub and the fluid source in a flow path extending between the hub and the fluid source. In some embodiments, the at least one filter may include a 0.2 μm filter. In some embodiments, the irradiation transmissive barrier may isolate the plurality of antimicrobial light emitters from the airflow channel.

In accordance with another example embodiment of the present disclosure, an example dispensing system for filling medical reservoirs may comprise a dispensing assembly. The dispensing assembly may comprise a housing including a plurality of fluid supply ports. The dispensing assembly may further comprise an end cap displaceable between a first position and a second position relative the housing via an actuator. The dispensing assembly may further comprise a compliant member intermediate the end cap and the housing. The dispensing assembly may further comprise a receptacle defined by the housing, end cap, and compliant member. The compliant member may be in a compressed state when the end cap is in the second position and a less compressed stated when the end cap is in the first position. The compliant member may deform into the receptacle in the compressed state. The dispensing assembly may further comprise a dispensing sharp extending into the receptacle and in communication with a sharp supply port of the fluid supply ports. The system may further comprise a clean air supply in selective communication with at least one of the fluid supply ports via a clean air valve. The system may further comprise a first steam supply in selective communication with at least one of the fluid supply ports via a first steam valve. The system may further comprise a second steam supply in selective communication with at least one of the fluid supply ports via a second steam supply valve.

In some embodiments, the first steam supply may include a boiler and a first supply pathway. In some embodiments, the second steam supply may include the boiler and a second supply pathway in communication with at least one heater. In some embodiments, the housing may include at least one pocket having a heating coil therein. In some embodiments, at least one of the at least one pocket may be in selective communication with one of the first stream supply and second steam supply via a steam jacket valve. In some embodiments, the dispensing sharp may include a pencil tip point and at least one side port. In some embodiments, the portion of the receptacle formed by the compliant member may be formed by an aperture through the compliant member. The diameter of the aperture may be smaller in the compressed state than in the less compressed state. In some embodiments, the first steam supply may be a saturated steam supply. In some embodiments, the second steam supply may be a superheated steam supply. In some embodiments, the first steam supply may be in selective communication with at least the sharp supply port. In some embodiments, the second steam supply may be in selective communication with at least the sharp supply port.

In accordance with another example embodiment of the present disclosure, an example a method of dispensing fluid into a medical reservoir may comprise flowing clean air through a receptacle of a fluid dispensing assembly. The method may further comprise displacing a port of the medical reservoir including a septum to a first position within the receptacle. The method may further comprise displacing an end cap of the dispensing assembly from a home position toward a main housing of the dispensing assembly and into a second position and distorting a compliant member intermediate the end cap and main housing to a compressed state. The method may further comprise forming a radial seal around the exterior of the port as the compliant member is distorted to the compressed state. The method may further comprise supplying a first stream to the receptacle for a first period of time. The method may further comprise supplying a second steam to the receptacle for a second period of time. The method may further comprise displacing the port to a spiked position in which a dispensing sharp of the receptacle extends through the septum and filling the bag with at least one fluid. The method may further comprise displacing the port out of the fluid dispensing assembly.

In some embodiments, the method may further comprise halting the flow of clean air to the receptacle and drawing a vacuum on the receptacle when the radial seal is formed. In some embodiments, the first steam may be a saturated steam. In some embodiments, the second steam may be a superheated steam. In some embodiments, the method may further comprise displacing the end cap from the second position to the home position after the second period of time elapses. In some embodiments, the method may further comprise heating at least the main housing with at least one heat source. In some embodiments, the at least one heat source may include at least one of a list consisting of one or more heater and a steam jacket. In some embodiments, the method may further comprise maintaining a steam jacket around at least a portion of the receptacle. In some embodiments, displacing the port to the spiked position may comprise piercing the septum with a pencil tip point of the dispensing sharp. Filling the bag with the at least one fluid may comprise dispensing the at least one fluid through at least one side port of the dispensing sharp into the medical reservoir via the port. In some embodiments, supplying the first steam to the receptacle may comprise supplying the first steam to the receptacle at least through a lumen of the dispensing sharp. In some embodiments, supplying the second steam to the receptacle may comprise supplying the second steam to the receptacle at least through a lumen of the dispensing sharp.

In accordance with another example embodiment of the present disclosure, an example air handling system for an environmentally controlled enclosure may comprise a pre-filter. The system may further comprise a blower downstream of and in communication with the pre-filter. The system may further comprise a series of turbulent kinetic energy dampeners having an upstream end in fluid communication with an output of the blower via a guide passage. Each of the turbulent kinetic energy dampeners in the series of turbulent kinetic energy dampeners being separated by a flow routing chamber. The system may further comprise a filtration chamber including a filter at the downstream end of the series of turbulent kinetic energy dampeners. The system may further comprise a supply plenum downstream of an output of the filter. The system may further comprise a processing chamber downstream of the supply plenum and partitioned therefrom by a supply diffusion grid. The supply diffusion grid may be no more than 150 mm from the output of the filter. The system may further comprise a first control zone in the processing compartment defined by an airflow channel extending from a first end of a dispensing assembly to an opposing end of the dispensing assembly. The first end may be in communication with a clean air output extending through the diffusion grid from the supply plenum. The opposing end of the airflow channel may be in direct communication with a supporting zone in the processing compartment. The system may further comprise a processing chamber exhaust separated from the processing compartment by an exhaust diffusion grid. The exhaust diffusion grid being at an end of the processing chamber opposite the supply diffusion grid.

In some embodiments, each of the turbulent kinetic energy dampeners in the series of turbulent kinetic energy dampeners may include an array of a plurality of elongate channels. In some embodiments, each of the plurality of elongate channels may have uniform cross-sectional shapes. In some embodiments, at least one of the turbulent kinetic energy dampener in the series of turbulent kinetic energy dampeners may include an array of a plurality of elongate channels each having a round cross-section shape. In some embodiments, the series of turbulent kinetic energy dampeners may include at least one upstream turbulent kinetic energy dampener and at least one downstream turbulent kinetic energy dampener. Each of the at least one upstream turbulent kinetic energy dampener may have an array of a plurality of elongate channels which each may have a first cross-sectional area. Each of the at least one downstream turbulent kinetic energy dampener may include an array of a plurality of elongate channels which each may have a second cross-sectional area less than the first cross-sectional area. In some embodiments, the series of turbulent kinetic energy dampeners may include at least one upstream turbulent kinetic energy dampener and at least one downstream turbulent kinetic energy dampener. Each of the at least one upstream turbulent kinetic energy dampener may have an array of a plurality of elongate channels which each may have a first cross-sectional shape. Each of the at least one downstream turbulent kinetic energy dampener may include an array of a plurality of elongate channels which each may have a second cross-sectional shape different from the first cross-sectional shape. In some embodiments, each of the flow routing chambers may redirect air flowing therethrough at least 90°. In some embodiments, a passage may extend through the clean air output. The passage may have a first cross-sectional area at an end of the clean air output in the supply plenum and a second cross-sectional area less than the first cross-sectional area at a second end of the clean air output adjacent the airflow channel. In some embodiments, the supporting zone may include the entirety of the processing compartment outside of the first zone. In some embodiments, the filter may be an ULPA filter and the pre-filter may have a MERV rating of at least 10. In some embodiments, the system may further comprise a transfer chamber having an input door and an output door. The input door may be intermediate the transfer chamber and the processing chamber. The output door may be intermediate the transfer chamber and an additional chamber. In some embodiments, the system may further comprise a transfer chamber plenum upstream of the transfer chamber and separated from the transfer chamber by a transfer chamber diffusion grid. The transfer chamber plenum may be communication with the supply plenum via a diversion valve assembly. In some embodiments, the diversion valve assembly may include a stator with at least one opening and a rotor with an arced cover. The rotor may be displaceable from a fully covering position in which the arced cover fully obstructs the at least one opening and a fully open position in which the arced cover obstructs no portion of the at least one opening. The may be a fluid communication path through the supply plenum to the transfer chamber plenum through a gap between the rotor and stator when the rotor is in the fully covering position. In some embodiments, the system may further comprise a transfer chamber and a transfer chamber plenum separated by a transfer chamber diffusion grid. The transfer chamber plenum may be separated from the supply plenum by a variable orifice valve. In some embodiments, the system may further comprise a transfer chamber exhaust separated from the transfer chamber by a transfer chamber exhaust diffusion gird. The transfer chamber exhaust may be isolated from fluid communication with the processing chamber exhaust between the transfer chamber exhaust diffusion grid to an outlet of the transfer chamber exhaust. In some embodiments, at least one of a first pressure sensor, first particulate sensor, and a first air flow rate sensor may be in fluid communication with the transfer chamber and at least one of a second pressure sensor, second particulate sensor, and second air flow rate sensor is may be in fluid communication with the transfer chamber exhaust downstream of the transfer chamber exhaust diffusion grid. In some embodiments, the transfer chamber may be separated from the processing chamber by an input door and may be separated from an additional chamber in the system by an output door. In some embodiments, at least one reservoir displacement assembly including a gripper may be disposed within the processing chamber and at least one reservoir retainer may be disposed in the transfer chamber. In some embodiments, the system may further comprises at least one drier upstream of the blower. In some embodiments, at least one of a first pressure sensor, first particulate sensor, and a first air flow rate sensor may be in fluid communication with the transfer chamber plenum and at least one of a second pressure sensor, second particulate sensor, and second air flow rate sensor is may be in fluid communication with the transfer chamber, at least one of a first pressure sensor, first particulate sensor, and a first air flow rate sensor may be in fluid communication with the supply plenum, at least one of a second pressure sensor, second particulate sensor, and second air flow rate sensor may be in fluid communication with the supporting zone. In some embodiments, at least one of a first pressure sensor, first particulate sensor, and a first air flow rate sensor may be in fluid communication with the processing chamber and at least one of a second pressure sensor, second particulate sensor, and second air flow rate sensor may be in fluid communication with the processing chamber exhaust downstream of the processing chamber exhaust diffusion grid.

In accordance with another example embodiment of the present disclosure, an example method of controlling an environment within an enclosure for a system for producing and packaging fluids may comprise displacing a flow of air drawn from a pre-filter through a series of alternating turbulent kinetic energy dampeners and flow routing chambers with a blower within a range of a prescribed flow rate. The method may further comprise displacing the flow of air through a filter intermediate a downstream end of the series of alternating turbulent kinetic energy dampeners and flow routing chambers and to a supply plenum for the enclosure. The method may further comprise displacing a first portion of the flow of air from the supply plenum, through a supply plenum diffusion grid, and into a supporting zone of a processing chamber. The method may further comprise establishing a first zone of environmental control in the supporting zone of the processing chamber. The method may further comprise displacing a second portion of the air from the supply plenum through a clean air output extending through the supply plenum diffusion grid. The clean air output may have a first end in communication the supply plenum and an opposing end in direct communication with a first end of an airflow channel in a dispensing assembly in the processing chamber. The method may further comprise establishing a second zone of environmental control in the processing chamber within the airflow channel. A second end of the airflow channel may be in direct communication with the supporting zone. The method may further comprise displacing at least some of the air flow from the processing chamber through an exhaust diffusion grid to processing chamber exhaust.

In some embodiments, the series of kinetic energy dampeners may have an upstream end in fluid communication with an output of the blower via a guide passage. In some embodiments, each of the turbulent kinetic energy dampeners may include an array of a plurality of elongate channels with uniform cross-sectional shapes. In some embodiments, the turbulent kinetic energy dampeners may include at least one upstream turbulent kinetic energy dampener and at least one downstream turbulent kinetic energy dampener. The cross-sectional shapes of the elongate channels in each of the at least one upstream turbulent kinetic energy dampeners may have a greater cross-sectional area than the cross-sectional shapes of the elongate channels in each of the at least one downstream turbulent kinetic energy dampeners. In some embodiments, each of the flow routing chambers may redirect air flowing therethrough at least 90°. In some embodiments, a passage may extend through the clean air output. The passage may have a first cross-sectional area at the first end of the clean air output and a second cross-sectional area less than the first cross-sectional area at the second end of the clean air output. In some embodiments, the filter may be an ULPA filter and the pre-filter may have a MERV rating of at least 10. In some embodiments, the method further may comprise at least one of drying the flow of air upstream of the blower and displacing the flow of air through a muffler upstream of the blower. In some embodiments, the method may further comprise displacing a third portion of the flow of air from the supply plenum, through a transfer chamber plenum and transfer chamber plenum diffusion grid into a transfer chamber. Ins some embodiments, the method may further comprise actuating a diversion valve assembly intermediate the supply plenum and the transfer plenum. In some embodiments, the method may further comprise establishing an aerodynamic boundary between the supply plenum and the transfer plenum with the diversion valve assembly. In some embodiments, the method may further comprise actuating the diversion valve assembly to a partially open state at no greater than a first predefined rate while opening a door intermediate the transfer chamber and processing chamber at no greater than a second predefined rate. In some embodiments, the method may further comprise actuating the diversion valve assembly to a fully closed position at no greater than a first predefined rate and subsequently opening a door intermediate the transfer chamber and another chamber in the enclosure. In some embodiments, the method may further comprise actuating the diversion valve assembly to a partially open state at no greater than a first predefined rate, opening a door intermediate the transfer chamber and another chamber in the enclosure at no greater than a second predefined rate, and actuating the diversion valve assembly to a fully open position at no greater than a third predefined rate. The third predefined rate may be greater than the first predefined rate. In some embodiments, the method may further comprise closing the door and actuating the diversion valve assembly to a fully open position for a recovery period. In some embodiments, the transfer chamber may be subjected to at least 50 air changes during the recovery period. In some embodiments, the method may further comprise actuating the diversion valve assembly to a fully open position at no greater than a predefined rate and subsequently spiking a port of an IV bag with a dispensing sharp in the dispensing assembly and filling the IV bag with a target volume of fluid. In some embodiments, the first environmental control zone may conform at least to ISO 7 cleanroom standards for non-viable particulate, flow velocity, air exchanges per unit time, and positive pressurization. In some embodiments, the second environmental control zone may conform at least to ISO 5 cleanroom standards for non-viable particulate, flow velocity, air exchanges per unit time, positive pressurization, and unidirectional airflow. In some embodiments, the average turbulent intensity in the second environmental control zone may be less than 2%. In some embodiments, the first environmental control zone may be subjected to at least 60 air exchanges a minute and the second environmental control zone may be subjected to at least 300 air exchanges a minute. In some embodiments, a distance between an output of the filter and the supply plenum diffusion grid may be no greater than 150 mm. In some embodiments, a distance between an output of the filter and a tip of a dispensing sharp included in the dispensing assembly may be no greater than 250 mm.

### BRIEF DESCRIPTION OF THE DRAWINGS

These and other aspects will become more apparent from the following detailed description of the various embodiments of the present disclosure with reference to the drawings wherein:

FIG. 1 depicts a perspective view of an exemplary system for producing an packaging fluids;

FIG. 2 depicts a top plan view of a portion of an example system;

FIG. 3 depicts a flowchart detailing a number of example actions which may be executed to package fluid into a reservoir;

FIG. 4A depicts a front view of an exemplary reservoir;

FIGS. 4B-C depict views of the exemplary reservoir of FIG. 4A with a concentrate contained within the reservoir;

FIG. 5 depicts a flowchart detailing a number of example actions which may be executed to fill a reservoir including at least one frangible with a fluid;

FIG. 6A depicts a front view of another exemplary reservoir;

FIG. 6B depicts a perspective view of the example reservoir of FIG. 6A with a concentrate therein;

FIG. 7A depicts a perspective view of another example reservoir having a liquid concentrate therein;

FIG. 7B depicts a perspective view of another example reservoir;

FIG. 8 depicts a flowchart detailing a number of example actions which may be executed to fill a reservoir with concentrate and remove at least some of the solvent from the concentrate through the sealed reservoir;

FIG. 9A depicts a view of an example reservoir including a partially solid concentrate;

FIG. 9B depicts a detailed view of a portion of an example reservoir including a partially solid concentrate;

FIGS. 10A-B depict perspective views of example projection bearing bodies which may, for instance, be coupled to ports of an IV bag;

FIG. 11 depicts a perspective view of a portion of an IV bag with projection bearing bodies coupled to ports of the bag;

FIG. 12 depicts a perspective view of a portion of an IV bag with projection bearing bodies coupled thereto being held by an automated grasper;

FIG. 13 depicts a perspective view of an example ribbing clip which may be coupled to ports of an IV bag;

FIGS. 14A-C depict views of an example ribbed ring which may be coupled to a port of an IV bag;

FIGS. 15A-B depict views of an example embodiment of a ribbed spacer which may be coupled to a port of an IV bag;

FIGS. 16A-C depict views of example graspers holding projection bearing bodies on ports of an IV bag;

FIGS. 17A-B depict cross-sectional views of example IV bag ports with projection bearing bodies coupled thereto;

FIGS. 18A-C depict views of example projection bearing bodies coupled to ports of IV bags;

FIGS. 19A-B depict views of example chaining linkages which may be used to couple IV bags to one another;

FIG. 20A depicts an example IV bag with an example chaining linkage coupled thereto;

FIG. 20B depicts a series of example IV bags coupled together with a number of example chaining linkages;

FIGS. 21A-F depict various views of an exemplary port for an IV bag;

FIG. 22 depicts a portion of an example chain of a number of example reservoirs;

FIG. 23A depicts a portion of another example chain of example reservoirs;

FIG. 23B depicts a detailed view of the indicated region of FIG. 23A;

FIG. 24A depicts a portion of another example chain of example reservoirs;

FIG. 24B depicts a detailed view of the indicated portion of FIG. 24A;

FIGS. 25A-B depict views of a number of example chains of example reservoirs;

FIG. 26 depicts a portion of another example chain of example reservoirs;

FIGS. 27A-B depict views of an example dispenser for a chain of reservoirs;

FIG. 28 depicts a cross-sectional view of an example dispenser;

FIG. 29 depicts a cross sectional view of an example dispenser;

FIG. 30A depicts a top plan view of another example dispenser;

FIG. 30B depicts a cross-sectional view taken at the indicated cut plane of FIG. 30A;

FIGS. 31-32 depict views of another example dispenser;

FIG. 33 depicts a perspective view of an example dispenser with a portion of the dispenser broken away to show a spool of an exemplary chain of reservoirs within the dispenser;

FIG. 34A depicts a dispenser with an example removable cover assembly in a locked state;

FIG. 34B depicts a dispenser with an example removable cover assembly in an unlocked state;

FIG. 34C depicts a partially exploded view of an example dispenser;

FIG. 35 depicts a rear view of an example backing body of a removable cover assembly;

FIG. 36A depicts a portion of an example removable cover assembly;

FIG. 36B depicts a cross-sectional view of an example removable cover assembly;

FIG. 37A depicts a perspective view of an example reel of a chain of reservoirs;

FIG. 37B depicts view of an end of an example chain of reservoirs coupled to an example spool;

FIG. 38A depicts a perspective view of the example dispenser of FIG. 34A without the removable cover assembly;

FIG. 38B depicts a detailed view of the indicated region of FIG. 38A;

FIG. 39 depicts a perspective view of an example spool which may be included in certain dispensers;

FIG. 40 depicts a view of a tensioner assembly exploded from an example spool;

FIGS. 41A-B depict exploded views of an example tensioner assembly;

FIG. 42 depicts a block diagram of an example docking assembly, an example sanitary interface assembly, and portion of a processing compartment of a system;

FIG. 43 depicts a view of an example docking assembly and example infeed plug assembly sealing an infeed aperture to a processing compartment;

FIG. 44 depicts a view of an example dispenser loaded into an example carriage of a docking assembly;

FIG. 45 depicts a view of an infeed aperture plug with a portion of the infeed aperture plug housing removed;

FIGS. 46A-B depict a flowchart detailing a number of example actions which may be executed to load and open a dispenser with a docking assembly and sanitary interface assembly;

FIG. 47 depicts a flowchart detailing a number of example actions which may be executed to remove a dispenser from a system;

FIGS. 48A-F depict a progression of block diagrams showing an example removable cover assembly being unlocked and positively retained by example projections of an example lock actuation assembly;

FIGS. 49A-D depict views of an example of a bag vending assembly;

FIGS. 50A-E depicts views of another example bag vending assembly;

FIGS. 51A-C depict views of another example bag vending assembly;

FIGS. 52A-B depict views of example bag vending assemblies coupled to an example housing portion of a dispenser for reservoirs;

FIG. 53 depicts a perspective view of an example dispenser;

FIG. 54 depicts a perspective view of an example frame and removable cover assembly which may be included in a dispenser;

FIG. 55 depicts a cross-sectional view of the example dispenser depicted in FIG. 53;

FIGS. 56A-57 depict views of example dispensers with an outer housing;

FIGS. 58A-B depict views of example dispensers with partial outer housings;

FIG. 59 depicts a perspective view of an example infeed aperture plug in place within an aperture in a wall of an environmentally controlled enclosure;

FIG. 60 depicts a cross-sectional view of an example infeed aperture plug;

FIG. 61 depicts a cross-sectional view of a portion of an example dispenser displacing toward an example infeed aperture plug;

FIG. 62 depicts a cross-sectional view of a port sealing assembly of an example infeed aperture plug in sealing contact with a frangible of a portion of an example dispenser;

FIG. 63 depicts a cross-sectional view of a portion of an example dispenser in a fully installed position against an example infeed aperture plug;

FIG. 64 depicts a cross-sectional view of a portion of an example infeed aperture plug with a cover of an example dispenser retained thereon displaced away from an aperture in a wall of an environmentally controlled enclosure;

FIG. 65 depicts a cross-sectional view of a portion of an example dispenser to which a cover of the dispenser has been reengaged by displacement of an example infeed aperture plug into sealing relation with an aperture in a wall of an environmentally controlled enclosure;

FIG. 66A depicts a cross-sectional view of an example infeed aperture plug and a example dispenser portion with a plunger of the infeed aperture plug driven to an ejecting position;

FIG. 66B depicts a cross-sectional view of an example infeed aperture plug and a portion of an example dispenser with the cover displaced to a position in which it is captured between an interior face of a frame of the dispenser and a set of retainers on the frame;

FIG. 67 depicts a view of a portion of an example used dispenser being displaced away from an example infeed aperture plug;

FIG. 68A depicts a top plan view of an example processing chamber including an example bag displacement assembly;

FIG. 68B depicts a view of an example bag displacement assembly within an example processing chamber of a system;

FIGS. 69A-70B depict views of various portions of bag retention assemblies that may be included in certain bag displacement assemblies;

FIG. 71 depicts a portion of an exemplary bag retention assembly including example jaw position sensors;

FIG. 72A-C depicts a views of portions of an example bag displacement assembly;

FIG. 73 depicts a flowchart detailing a number of example actions which may be executed to fill and displace a reservoir through certain example systems;

FIG. 74 depicts a perspective view of an example reservoir singulating assembly which may be used to individualize a reservoir from a chain of reservoirs;

FIGS. 75A-C depict views of an example splitter assembly which may be included in certain reservoir singulating assemblies;

FIGS. 76A-D depict views of example splitters which may be included in certain reservoir singulating assemblies;

FIG. 77 depicts a bag retention assembly grasping a bag from a bag presenter of an example dispenser;

FIG. 78 depicts an example chain of bags pulled from a dispenser by an exemplary bag retainer assembly;

FIG. 79A depicts an example bag retainer assembly pressing ports of a bag into port retainers of an example fill station;

FIG. 79B depicts an example bag retainer assembly spaced from a bag held in port retainers of an example fill station;

FIG. 80 depicts an example bag retainer assembly aligned with ports of a bag adjacent a bag singulating assembly;

FIGS. 81A-B depict views of an example splitter assembly of an example bag singulating assembly separating a bag from an example chain of bags;

FIG. 82 depicts a bag in place at an example fill station after being individualized from an example chain;

FIG. 83 depicts a flowchart detailing a number of example actions which may be executed to singulate a bag from a chain of bags;

FIG. 84 depicts a flowchart detailing a number of example actions which may be executed in the event a fault is encountered during individualization of a bag from a chain;

FIG. 85 depicts a view of a portion of an exemplary system including an example bag chain orientation sensor;

FIG. 86 depicts a flowchart detailing a number of example actions which may be executed to adjust the orientation of a chain of bags spooling out of a dispenser;

FIGS. 87A-D depict a series of illustrative representations of images from an imager type chain orientation sensor;

FIG. 88 depicts a representational diagram of a chained bag being fed into a processing chamber at a slight angle;

FIG. 89 depicts an example dispenser assembly which may be included in certain fill stations;

FIG. 90 depicts a block diagram of a portion of an example filling station including an example cartridge;

FIG. 91 depicts another example cartridge and irradiation assembly;

FIG. 92 depicts a view of the example cartridge and irradiation assembly of FIG. 91 with a portion of the cartridge removed;

FIG. 93 depicts an example filter assembly which may be included in an example cartridge;

FIG. 94 depicts a view through an example air flow channel which may be included in a cartridge;

FIG. 95 depicts a view of a portion of an example fill station;

FIG. 96 depicts a flowchart detailing a number of example actions which may be executed to install a cartridge in an example fill station and fill a reservoir at the fill station;

FIG. 97A depicts a perspective view of another example cartridge;

FIG. 97B depicts an exploded view of the example cartridge of FIG. 97A;

FIGS. 98A-B depict views of portions of example cartridges;

FIG. 99A depicts a perspective view of another example cartridge;

FIG. 99B depicts an exploded view of the example cartridge of FIG. 99A;

FIGS. 100A-B depict exploded views of an example filter assembly which may be included in a cartridge;

FIG. 101A depicts a perspective view of another example cartridge;

FIG. 101B depicts an exploded view of the example cartridge of FIG. 101A;

FIG. 102A depicts a bottom plan view of an example cartridge;

FIG. 102B depicts a cross-sectional view taken at the indicated cut plane of FIG. 102A;

FIGS. 103A-C depict a number of cross sectional views of the example cartridge of FIG. 101A;

FIG. 104 depicts a flowchart detailing a number of example actions which may be executed to install a cartridge in a filling station;

FIG. 105 depicts an exemplary filling station;

FIG. 106 depicts a perspective view of an example cartridge mount which may be included in a filling station;

FIG. 107 depicts an example filling station with a cartridge installed therein;

FIG. 108A depicts a perspective view of an example cover removal assembly for removing a cover body from a cartridge;

FIG. 108B depicts an exploded view of an example cover removal assembly;

FIG. 109 depicts a cross-sectional view of an example fill station in a cartridge exchange state with a cartridge installed therein;

FIG. 110 depicts a cross-sectional view of an example fill station with a cartridge displaced to an access position;

FIG. 111A depicts a cross-sectional view of example sealing member which may be included in a cover removal assembly in place against a wall of an enclosure portion of a system;

FIG. 111B depicts the example sealing member and enclosure of FIG. 111A with an example cartridge in an access position;

FIG. 112 depicts a cross-sectional view of an example cartridge in an access position at an example fill station with a cover body for the cartridge removed by an example cover removal assembly;

FIG. 113 depicts a cross-sectional view of an example cartridge in an access position at an example fill station with a cover engagement assembly of an example cover removal assembly in an engaged state with the cover of the cartridge;

FIG. 114 depicts a cross-sectional view of an example cartridge in an access position at an example fill station with a cover of the cartridge displaced out of engagement with the cartridge by an example cover removal assembly;

FIGS. 115A-B depict views of an example clean air output;

FIG. 116 depicts a perspective view of an example clean air output and an clean air output actuator;

FIG. 117 depicts a perspective view of a portion of an enclosure;

FIG. 118 depicts a cross-sectional view of an example fill station, cartridge, and clean air output;

FIG. 119 depicts a cross-sectional view of an example clean air output with a yoke of a clean air output actuator magnetically coupled to the clean air output through the wall of the enclosure;

FIG. 120 depicts an exploded view of a portion of an example cover removal assembly;

FIG. 121 depicts a cross-sectional view of an example cartridge, filling station, and cover removal assembly;

FIG. 122 depicts a cross-sectional view of a sealing member of an example cover removal assembly compressed against a rim surrounding a fenestration in an enclosure;

FIG. 123 depicts a cross-sectional view of a sealing member of an example cover removal assembly compressed against a rim surrounding a fenestration in an enclosure and a sealing member of an example cartridge compressed against an opposing surface of the enclosure and surrounding the fenestration;

FIG. 124 depicts a cross-sectional view of an example cartridge in an access position at an example fill station with a cover body for the cartridge removed by an example cover removal assembly;

FIG. 125 depicts a perspective view of an example fill station with an irradiation assembly displaced adjacent a portion of a cartridge;

FIG. 126A depicts a cross-sectional view of a port of an example cartridge being displaced into a receptacle of an example supply manifold;

FIG. 126B depicts a cross-sectional view of a port of an example cartridge further displaced into the receptacle of the example supply manifold;

FIG. 127 depicts a flowchart detailing a number of example actions which may be executed to prime a cartridge installed in a filling station;

FIG. 128 depicts an example priming line;

FIG. 129 depicts another example priming line;

FIG. 130 depicts an example fill displacement stage;

FIG. 131 depicts an example filling station with a priming line in a port retainer of a fill displacement station of the example filling station;

FIG. 132 depicts a priming line advanced into an example cartridge such that the dispensing sharp of the example cartridge extends into the priming line;

FIG. 133 depicts a portion of an example fill station with a bag retained in place by port retainers of the fill displacement stage of the example fill station;

FIG. 134 depicts an example irradiation assembly, irradiation assembly actuator, and priming line retainer with an example priming line therein;

FIG. 135 depicts an example fill displacement stage having a priming line retainer;

FIG. 136 depicts a flowchart detailing a number of example actions which may be executed to fill a bag at an example filling station;

FIG. 137 depicts a flowchart detailing a number of example actions which may be executed to irradiate a port and septum of a bag, portion of a priming line, and/or dispensing sharp of a cartridge with an irradiation assembly.

FIG. 138 depicts a cross-section of an example fill station with a port of a bag advanced into an irradiation position within an example cartridge in place at the filling station;

FIG. 139 depicts a bag being filled via an example cartridge at an example filling station;

FIG. 140 depicts a flowchart detailing a number of example actions which may be executed to remove a cartridge from a filling station;

FIG. 141 depicts a perspective view of an example filling station;

FIGS. 142A-142C-depict view of an example dispensing assembly which may be included in various example filling stations;

FIGS. 143A-F depict views of another example dispensing assembly which may be included in various example filling stations;

FIG. 144A depicts a top plan view of an example processing compartment and transfer chamber which may be included in a system;

FIGS. 144B-C depict an example bag retention assembly grasping a filled bag at an example filling station;

FIG. 145 depicts a filled bag displaced into an example transfer chamber by an example bag retention assembly;

FIG. 146 depicts an example bag retainer;

FIGS. 147-148 depict a filled bag being handed off from an example bag retention assembly to an example bag retainer in an example transfer chamber;

FIG. 149 depicts a filled bag retained on an example bag retainer and isolated within an example transfer chamber;

FIG. 150 depicts a gripper assembly of a gantry assembly in an outfeed chamber of a system grasping a filled bag on an example bag retainer in a transfer chamber;

FIG. 151 depicts a filled bag held in a gripper assembly of a gantry assembly;

FIG. 152A-C depict an example mix assisting assembly;

FIGS. 153A-B depict an example mix assisting assembly;

FIG. 154A depicts a rear perspective view of the example mix assisting assembly of FIG. 153A with a rear panel removed;

FIG. 154B depicts a detailed view of the indicated region of FIG. 154A;

FIG. 155A depicts a view of an example mix assisting assembly including doors which are in an open state;

FIG. 155B depicts a rear view of the example mix assisting assembly of FIG. 155A;

FIG. 156A depicts a filled bag in place within an example mix assisting assembly;

FIG. 156B depicts a rear view of the example mix assisting assembly of FIG. 155A with the doors in a closed state;

FIG. 157 depicts a perspective view of an example door which may be included in a mix assisting assembly;

FIG. 158 depicts a flowchart detailing a number of example actions which may be executed to mix contents of a reservoir with a mix assisting assembly of the type shown in FIG. 155A;

FIG. 159 depicts a block diagram of an example particulate inspection system;

FIG. 160A depicts a perspective view of an example particulate inspection system;

FIG. 160B depicts a perspective view of an example particulate inspection system with a bag type reservoir installed therein;

FIG. 160C depicts a cross-sectional view of an example particulate inspection system;

FIG. 161A depicts a perspective view of an example external illuminator which may be included in an example particulate inspection system;

FIG. 161B depicts a cross-sectional view of an example external illuminator which may be included in an example particulate inspection system;

FIG. 161C depicts a detailed view of the indicated region of FIG. 161B;

FIG. 162 depicts a flowchart detailing a number of example actions which may be executed to conducted a functionality check for an example particulate inspection system;

FIG. 163 is a representation of an image of a reservoir against a background;

FIG. 164A-F are representations of images of a number of respective different content types within reservoirs;

FIG. 165A depicts a representation of an image of a reservoir in a particulate inspection system;

FIG. 165B depicts an enlarged view of a portion of FIG. 165A;

FIGS. 166A-B depict diagrams of collimated light passing through a bubble;

FIG. 167A depicts an illustrative representation of a simulated image of an illuminated bubble in a reservoir;

FIG. 167B depicts an illustrative representation of a simulated image of an illuminated bubble in a reservoir;

FIG. 168 depicts a diagram of an example bubble illuminated with collimated light;

FIG. 169A depicts an illustrative representation of a simulated image of an illuminated bubble in a reservoir;

FIG. 169B depicts an illustrative representation of a simulated image of an illuminated bubble in a reservoir;

FIG. 170A depicts an illustrative representation of a simulated image of an illuminated bubble in a reservoir;

FIG. 170B depicts an illustrative representation of a simulated image of an illuminated bubble in a reservoir;

FIGS. 171A-B depict an illustrative representation of a simulated image of an illuminated opaque white particle against a chessboard patterned background;

FIGS. 172A-B depict an illustrative representation of a simulated image of an illuminated opaque reflective particle against a chessboard patterned background;

FIG. 173A-B depict an illustrative representation of a simulated image of an illuminated particle against a chessboard patterned background, the particle being a material which is opaque and dark in color;

FIG. 174A-B depict an illustrative representation of a simulated image of an illuminated transparent particle against a chessboard patterned background;

FIG. 175A-D depict a progression of image representations of a reservoir in which a bubble is displacing over a period of time;

FIG. 176 is a representation of an image of a reservoir in place in a particulate inspection system;

FIG. 177 depicts a representation of an image of a reservoir having a number of various pieces of particulate therein;

FIGS. 178A-D depict a progression of image representations in which a piece of particulate is displacing within a reservoir of time;

FIG. 179 depicts a data flow diagram for a particulate inspection system;

FIG. 180 depicts a flowchart detailing a number of example actions which may be executed to inspect a reservoir with a particulate inspection system and route the reservoir through a system based on the inspection result;

FIG. 181 depicts a flowchart detailing a number of example actions which may be executed to detect, track, and classify regions of interest within a reservoir and generate a pass/fail determination for the reservoir;

FIG. 182 depicts a flowchart detailing a number of example actions which may be executed to detect regions of interest within a reservoir;

FIG. 183 depicts a representation of a raw image of a reservoir in place at a particulate inspection system;

FIG. 184A depicts a representation of a foreground segmented image of a reservoir in place in a particulate inspection system;

FIGS. 184B-C depict enlarged views of the indicated regions in FIG. 184A;

FIG. 185 depicts a representation of an example image of edge detections after a morphological transformation;

FIG. 186A depicts a representation of a near edge filtered foreground segmented image;

FIG. 186B-C depict enlarged views of the indicated regions in FIG. 186A;

FIG. 187 depicts a representation of the image of FIG. 186A after a morphological transformation;

FIG. 188 depicts a representation of the image of FIG. 187 with pixel clusters having an area greater than a predefined threshold removed;

FIG. 189A depicts a representation of the image of FIG. 188 after a morphological transformation;

FIGS. 189B-C depict enlarged views of the indicated regions in FIG. 189A;

FIG. 190A depicts a representation of the image of FIG. 183 with bounding boxes applied around detected regions of interest;

FIG. 190B depicts an enlarged view of the indicated region of FIG. 190A;

FIG. 191A depicts a representation of an image of a reservoir in place at a particulate inspection systems taken some period of time after the image of FIG. 183 with bounding boxes applied around detected regions of interest;

FIG. 191B depicts an enlarged view of the indicated region of FIG. 191A;

FIG. 192 depicts a flowchart detailing a number of example actions which may be executed to track regions of interest over a series of frames of a reservoir in place in a particulate inspection system;

FIGS. 193A-D depict a region of interest track as it grows over a series of frames;

FIG. 194 depicts the track of FIGS. 193A-D overlaid on a representation of a portion of an image of a reservoir against a background pattern;

FIG. 195 depicts a flowchart detailing a number of example actions which may be executed to score and classify regions of interest detected in a reservoir in place at a particulate inspection system;

FIG. 196A depicts an example annotated image of a reservoir in place at a particulate inspection system showing a number of tracks for detected regions of interest;

FIG. 196B depicts an example annotated image of a reservoir in place at a particulate inspection system showing a number of tracks for detected regions of interest;

FIG. 197 depicts a flowchart detailing a number of example actions which may be executed to identify and categorize objects in a reservoir;

FIG. 198 depicts a flowchart detailing a number of example actions which may be executed to identify and categorize objects in a reservoir;

FIG. 199 depicts an example data flow diagram for an example particulate inspection system;

FIG. 200 depicts a block diagram of an example marking assembly which may be included in a system;

FIG. 201 depicts a perspective view of an example marking assembly;

FIG. 202 depicts another perspective view of a portion of a marking assembly including an embossing head;

FIG. 203 depicts a perspective view of a portion of a marking assembly including an embossing head;

FIG. 204 depicts a block diagram of an example embossing head;

FIG. 205 depicts a block diagram of an example embossing head;

FIG. 206 depicts a side view of an example rotor which may be included in an embossing head;

FIG. 207 depicts a flowchart detailing a number of example actions which may be executed to place a string of marking bodies in desired relative positions in an embossing head;

FIGS. 208A-B depict exploded views of an example embossing head;

FIGS. 209A-B depict exploded views of a portion of an example embossing head;

FIG. 210 depicts a side view of an example rotor which may be included in an example embossing head;

FIG. 211 depicts a cross-sectional view of a portion of an example embossing head;

FIGS. 212A-B depict exploded views of a portion of an example embossing head;

FIG. 213A depicts a perspective view of an imager viewing a marked region of a bag held by a grasper;

FIG. 213B depicts a representation of an image of a marked region of a bag captured by the imager such as that shown in FIG. 213A;

FIG. 213C depicts a representation of an analyzed image of a marked region of a bag which may be included in a log for the respective bag;

FIG. 214 depicts a block diagram of an example outfeed assembly which may be included in a system;

FIGS. 215-217 depict views of a portion of an outfeed assembly including drawers;

FIG. 218 depicts an exploded view of an example drawer slide assembly;

FIGS. 219A-C depict views of an example drawer slide assembly;

FIG. 220 depicts a flowchart detailing a number of example actions which may be executed to distribute bags from a drawer of an outfeed assembly;

FIG. 221 depicts a top plan view of a portion of a system showing an outfeed assembly of the system partially filled with reservoirs;

FIG. 222 depicts a gantry assembly positioning a reservoir over an example access controlled reservoir receptacle of an example outfeed assembly;

FIG. 223 depicts an example outfeed assembly including a set of drawers and a separate access controlled receptacle for rejected reservoirs;

FIG. 224 depicts a block diagram of a portion of an example outfeed assembly;

FIGS. 225A-B depict representative illustrations of example images from outfeed inventory imagers which may be included example outfeed assemblies;

FIGS. 226A-B depict representational illustrations of processed images from outfeed inventory imagers which may be included in example outfeed assemblies;

FIG. 227 depicts an outfeed inventory sensor and an example set of receptacles for receiving reservoirs of an example system for producing and packaging fluids;

FIG. 228 depicts an example data flow diagram for generating various records relating to packing of fluid into reservoirs;

FIGS. 229A-C depict a block diagrams of portions of a fluid handling system which may be included in example systems for producing and packaging fluids;

FIG. 230A depicts a perspective view of an example accumulator reservoir;

FIG. 230B depicts a cross-sectional view of the example accumulator reservoir of FIG. 230A;

FIG. 231 depicts an example control diagram for an accumulator heater of a fluid handling system;

FIG. 232 depicts an example control diagram of a filter inlet pump of a fluid handling system;

FIG. 233 depicts an example control diagram for a return flow valve of a fluid handling system;

FIG. 234 depicts a flowchart detailing a number of example actions which may be executed to transfer a target amount of fluid into a bag;

FIG. 235 depicts a flowchart detailing a number of example actions which may be executed to dispense fluid into a bag in accordance with a calculated overall fill target;

FIG. 236 depicts a flowchart detailing a number of example actions which may be executed to generate and circulate a disinfect fluid in a fluid handling system;

FIG. 237 depicts a flowchart detailing a number of example actions which may be executed to install and prime a cartridge at a fill station;

FIG. 238 depicts a flowchart detailing a number of example actions which may be executed to disinfect a cartridge installed in a fill station;

FIG. 239 depicts a flowchart detailing a number of example actions which may be executed to actively cool a fluid handling system after a completing or initiating a heat disinfect;

FIG. 240 depicts a block diagram of an example pneumatic distribution assembly for an example system;

FIG. 241 depicts a block diagram of an example air preparation assembly which may be included in a pneumatic distribution assembly;

FIG. 242 depicts a block diagram depicting an exemplary set of pneumatic valve banks associated with example components of a system;

FIG. 243 depicts a block diagram of an example negative pressure distribution assembly which may be included in a pneumatic distribution system;

FIG. 244 depicts a block diagram of an example air handling assembly which may be included in example systems for producing and packaging fluids;

FIG. 245 depicts a perspective view of a portion of an example system including an air handling assembly;

FIG. 246-248 depict a series of cross-sections through the portion of the system depicted in FIG. 245;

FIG. 249 depicts a view of an example diversion valve assembly which may be included in an air handling assembly of examples systems for producing and packaging fluids;

FIG. 250A-B depict views of an example diversion valve assembly which may be included in an air handling assembly of examples systems for producing and packaging fluids;

FIG. 251-253 depict a series of cross-sections through the portion of the system depicted in FIG. 245;

FIG. 254 depicts a flowchart detailing a number of example actions which may be executed to place the interior of a dispenser into communication with a controlled environment in a processing chamber;

FIG. 255 depicts a flowchart detailing a number of example actions which may be executed to fill a bag with a dispensing assembly

FIG. 256 depicts a flowchart detailing a number of example actions which may be executed to transfer a filled bag into a transfer chamber from a processing chamber;

FIG. 257. depicts a flowchart detailing a number of example actions which may be executed to transfer a bag out of a transfer chamber to an outfeed compartment; and

FIG. 258 depicts another flowchart detailing a number of example actions which may be executed to transfer a bag out of a transfer chamber to a processing compartment.

### DETAILED DESCRIPTION

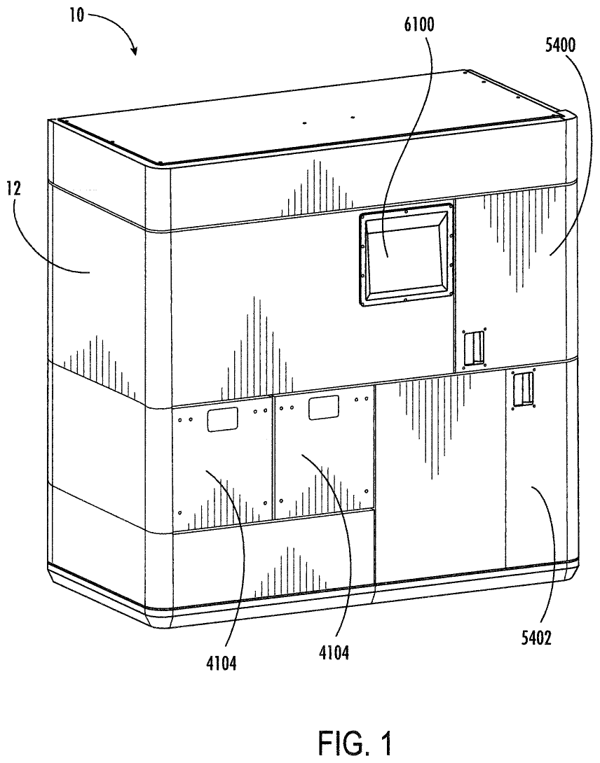

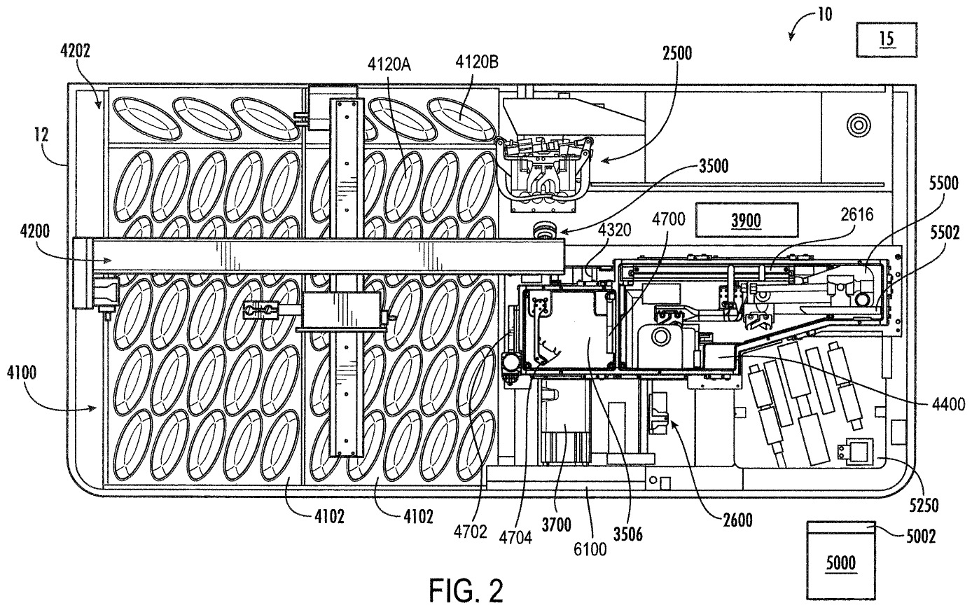

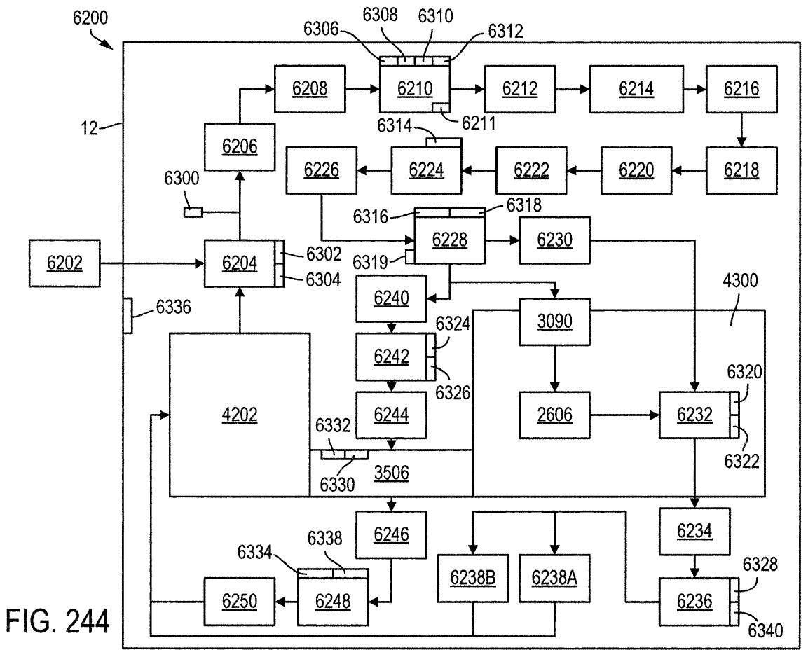

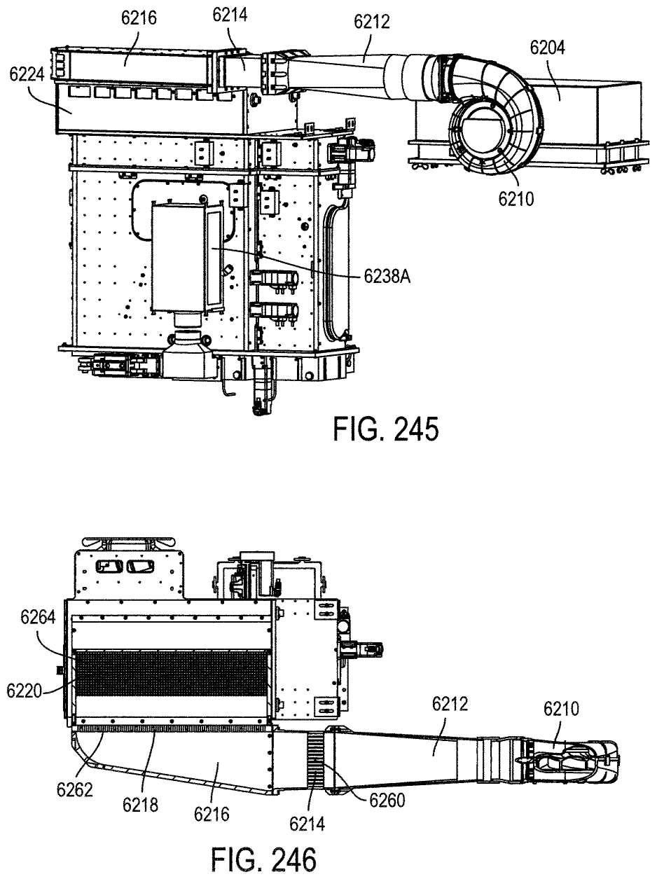

Referring now to FIGS. 1-2, a perspective view of an example system 10 and top plan view of a portion of an example system 10 are respectively depicted. As shown, a system 10 may include an enclosure 12 with a number of compartments 4300, 3506, 4202 and an exterior wall. The system 10 may also include an air handling assembly 6200 (see, e.g., FIG. 245) disposed above the compartments which is not depicted in FIG. 2 in order to better illustrate the interior compartments 4300, 3506, 4202. The air handling assembly 6200 may supply a flow of clean (e.g. HEPA or ULPA filtered air) to the interior compartments of the enclosure 12. The air may be conditioned to flow in laminar fashion and may be provided and routed through the enclosure 12 to satisfy minimum air flow rate, pressure, and particulate criteria for the various compartments or zones within each compartment. An example air supply assembly 6200 is described in greater detail in relation to FIG. 244. The enclosure 12 may include various doors 5400, 5402 to load or replace consumable components of the system 10. The enclosure 12 may also include one or more outfeed access through which filled reservoirs may be collected from the system 10. Two outfeed drawers 4102 are shown in the example embodiment.

The system 10 may receive bags 26 from a dispenser 5000 which are supplied in a state in which they are coupled to one another. The dispenser 5000 may be loaded into a system 10 via a docking assembly 5250 which may be accessed by unlocking and opening a docking assembly door 5400 of the enclosure 12. The dispenser 5000 may be sealed and terminally sterilized (e.g. ethylene oxide, e-beam, gamma irradiation, autoclaved, etc.). The dispenser 5000 may be displaced by the docking assembly 5250 relative to the enclosure 12. The system 10 may also include a sanitary interface assembly 5500. With the dispenser 5000 displaced to an access position relative to the enclosure 12, the sanitary interface assembly 5500 may expose the interior of the dispenser 5000 to a processing compartment 4300 of the enclosure 12. This may be accomplished by separating a removable cover assembly 5002 or cap from the dispenser 5000. The processing compartment 4300 may be environmentally controlled. The sanitary interface assembly 5500 may ensure that the dispenser 5000 is accessed in a manner which does not adversely impact the environment within the processing chamber 4300.

In certain examples, the sanitary interface assembly 5500 may include an infeed aperture plug 5502. Example infeed aperture plugs 5502 may also be referred to herein as dispenser outlet cover interfaces. When the removable cover assembly 5002 and infeed aperture plug 5502 are brought into contact with one another, seals may be formed. The seals may isolate surfaces of a removable cover assembly 5002 and infeed aperture plug 5502 which are exposed to the ambient, surrounding environment. The removable cover assembly 5002 and infeed aperture plug 5502 may also couple to one another (e.g. via application of a vacuum to a sealed volume between the two). The sanitary interface assembly 5500 may displace the removable cover assembly 5002 in tandem with the infeed aperture plug 5502 to a stowed position within the processing compartment 4300. This may establish communication between the interior of the dispenser 5000 and the environment of the processing compartment 4300. This may be accomplished without exposing the processing chamber 4300 or interior of the dispenser 5000 to components previously in communication with the external environment. The infeed aperture plug 5502 is shown in the stowed position in FIG. 2 for sake of illustration. Typically, the infeed aperture plug 5502 would be sealingly positioned against the processing chamber wall 4301 of the enclosure 12 when a dispenser 5000 is not installed in the system 10.

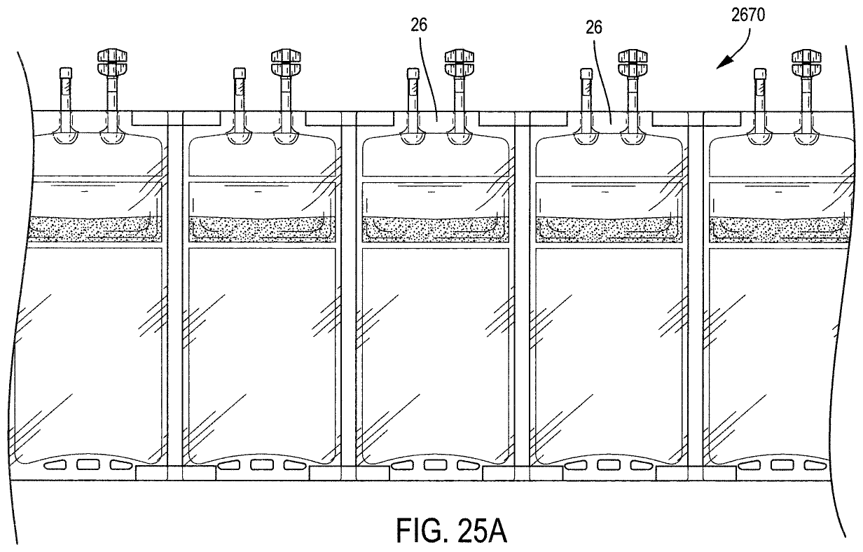

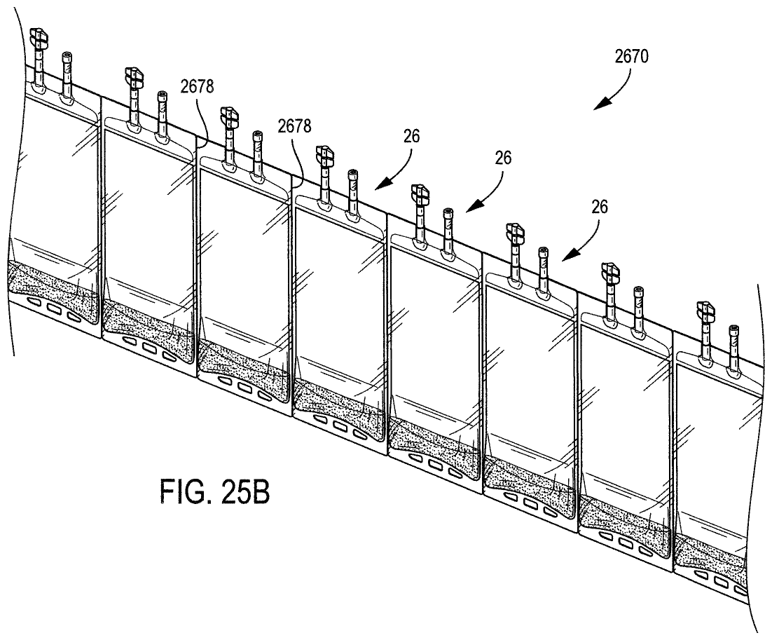

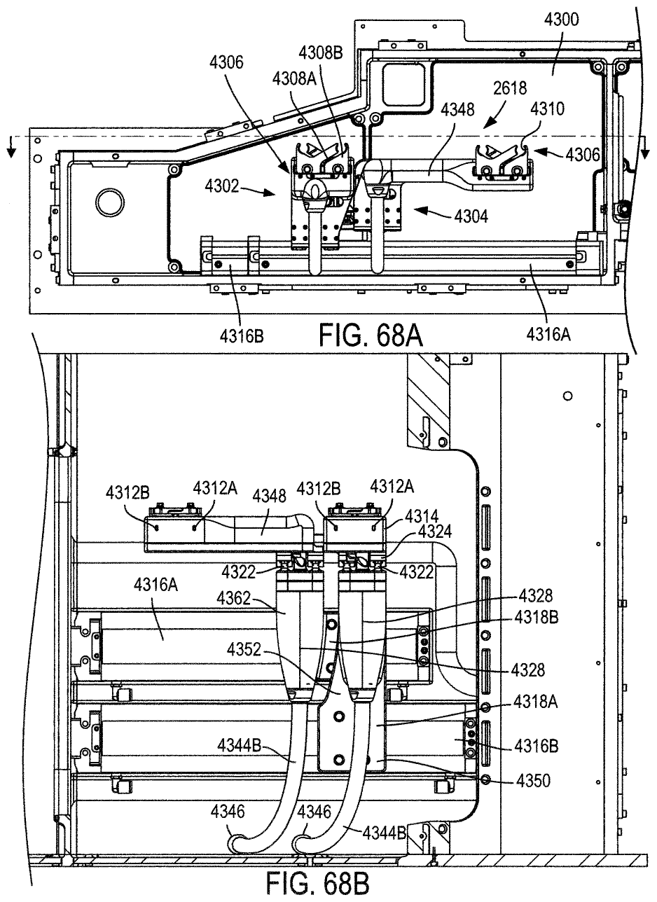

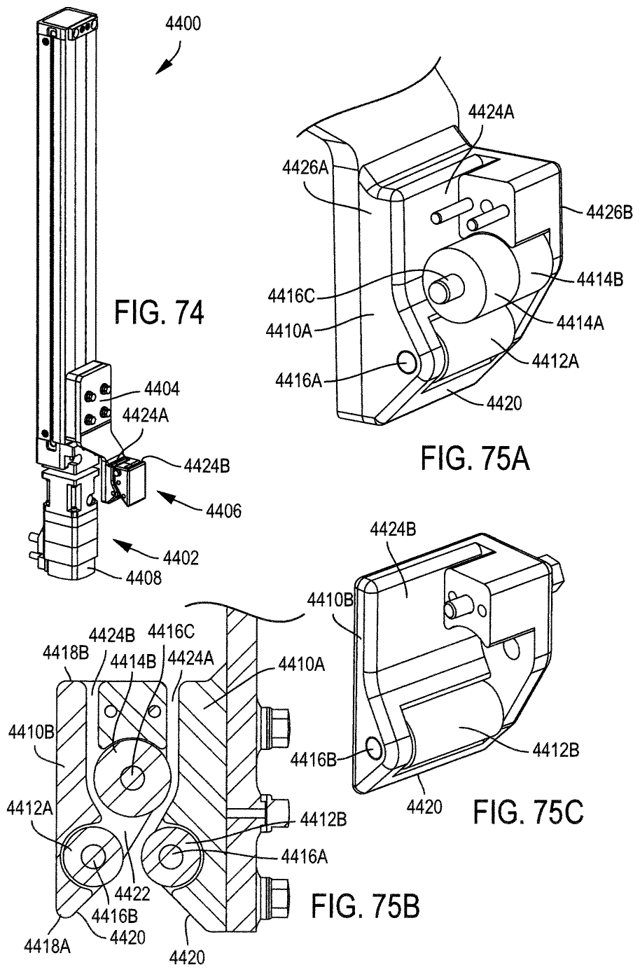

A bag displacement assembly 2618 disposed in the processing compartment 4300 may include one or more bag retainers (e.g. pneumatic graspers) disposed on a respective displacement stage (see, e.g., FIG. 68A). A filling station 2600 may also be included in the processing compartment 4300 and may include at least one bag retainer on a displaceable stage (see, e.g. FIG. 131). The bag displacement assembly 2618 may be actuated to collect and displace a chain 2670 (see, e.g., FIG. 25B) of bags 26 out of the dispenser 5000 and into the processing chamber 4300. Bags 26 may be individualized from the chain 2670 at a singulating assembly 4400 (see, e.g., FIG. 74).

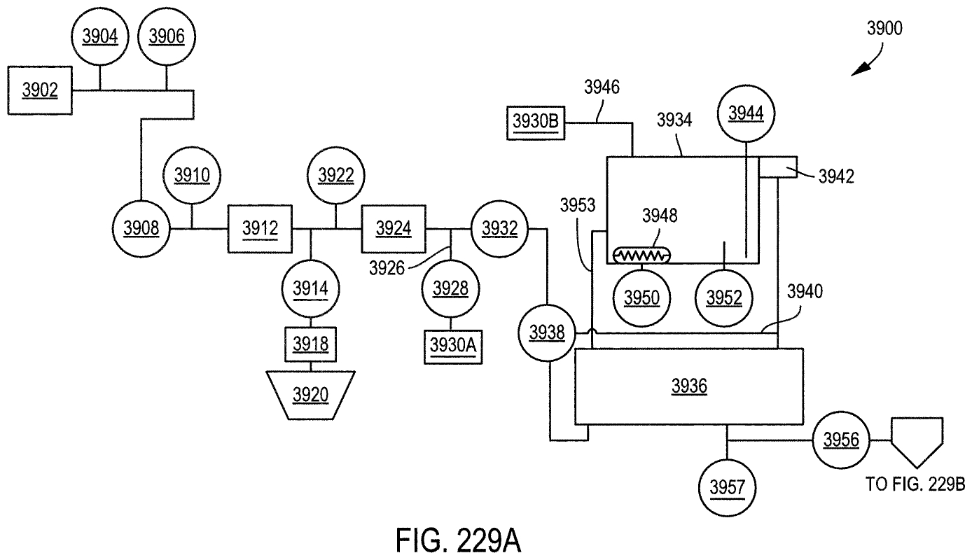

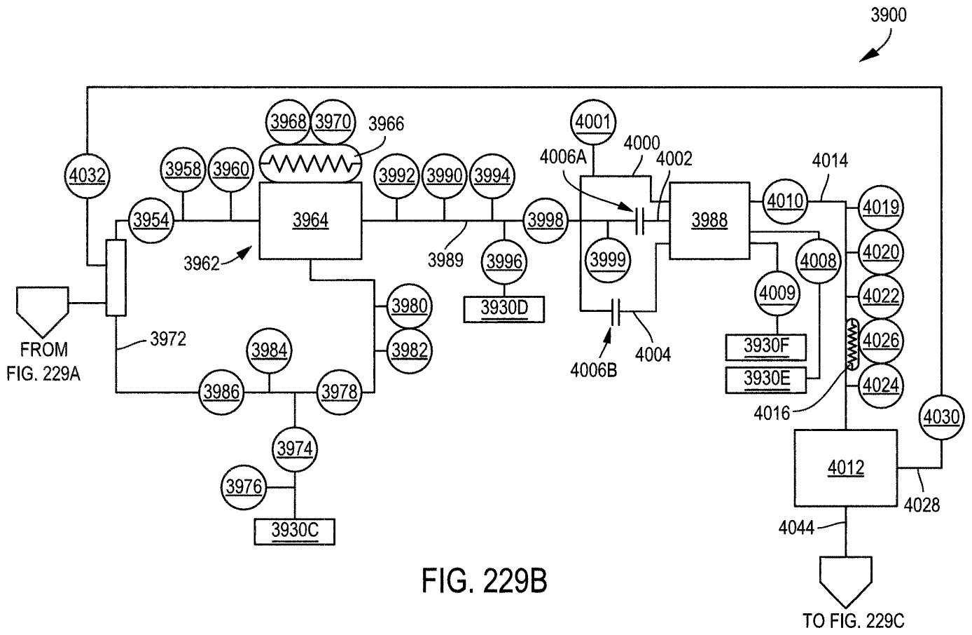

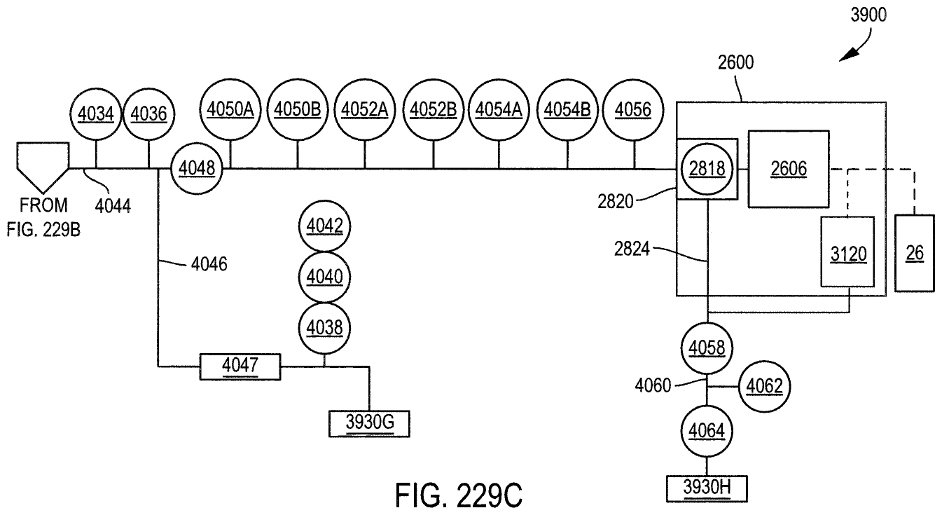

The filling station 2600 may be in fluid communication with a fluid handling system 3900 (see, e.g., FIGS. 229A-C) which provides water or diluent meeting predetermined quality criteria to the filling station 2600. For example, the fluid handling system 3900 may generate pharmacopeia grade water (e.g. Water for Injection quality water or WFI) for the filling station 2600. The fluid handling system 3900 may also precisely meter fluid supplied to the filling station 2600 to fill a predetermined target volume of fluid into bags 26 resident at the filling station 2600. In alternative examples, a fluid handling system 3900 which mixes a fluid may be included. Any fluid mixing arrangements described in U.S. Pat. No. 11,965,766, issued Apr. 23, 2024, entitled Medical Treatment System and Methods Using a Plurality of Fluid Line (Attorney Docket No. 00101.00343.Z55) or U.S. Pat. No. 11,980,587, issued May 14, 2025, entitled Systems, Methods, and Apparatuses for Producing and Packaging Fluids (Attorney Docket No. 00101.00325.AA697) each of which being incorporated by reference herein in its entirety may be used. Once a bag 26 has been filled and singulated, the bag 26 may be collected by the bag displacement assembly 2618 and displaced into a transfer chamber or compartment 3506.

Various hardware and components in communication with the processing compartment 4300 (and other compartments of the enclosure 12) may be selected to be cleanroom appropriate. For example, any fastener may be cleanroom rated. Components with moving parts may be selected to be cleanroom rated. Pneumatic actuation may be used where possible. Motors may either be cleanroom rated, positioned outside of the processing compartment 4300, or in environmentally sealed housings within the processing compartment 4300. In the latter cases, motion from the motors may be transmitted into the processing compartment 4300 through an environmentally sealed interface. Pneumatic lines, electrical lines, and other cabling may be bundled within an exterior sleeve to facilitate wipe down and may exit or enter the processing compartment 4300 (where needed) through hygienic glands. Such glands may be contoured to facilitate wipe down. Additionally, the walls 4301 defining the processing compartment 4300 may include rounded corners. Components may be selected to be suitable for any cleaning/antimicrobial agents they may be exposed to. For example, components may be compatible with repeated exposure to vaporized hydrogen peroxide (VHP). Aluminum components, for instance may all be anodized where exposure to VHP is possible. A vacuum line may be routed to or adjacent to any potential sources of particulate.

The example transfer chamber 3506 may include a first door 4700 and a second door 4702. The first door 4700 may be opened and closed to establish communication between the transfer chamber 3506 and the processing chamber 4300. The second door 4702 may be opened and closed to establish communication between the transfer chamber 3506 and an outfeed chamber 4202 of the enclosure 12. Each door 4700, 4702 may form an airtight seal when in a closed state which inhibits fluid communication between the transfer chamber 3506 and the respective adjacent compartment, 4300, 4202.

A bag retainer 4704 may be included in the transfer chamber 3506. With the first door 4700 of the transfer chamber 3506 in an open state, the bag displacement assembly 2618 may be actuated to displace a bag 26 into the transfer chamber 3506. The bag displacement assembly 2618 may hand the bag 26 off to the bag retainer 4704 and may subsequently be displaced out of the transfer chamber 3506. The first door 4700 may be closed isolating the bag 26 within the transfer chamber 3506 on the bag retainer 4704. The second door 4202 may be opened and a gantry assembly 4200 may be displaced to retrieve the bag 26 from the bag retainer 4704. The gantry assembly 4200 may remove the bag 26 from the transfer chamber 3506 and displace it into the outfeed chamber 4202. With the bag 26 removed from the transfer chamber 3506, the second door 4704 may be closed. Each time a door 4700, 4702 is opened and closed, the transfer chamber 3506 may be isolated and air may be flowed through the transfer chamber 3506. The system 10 may ensure at least a certain number of air exchanges within the transfer chamber 3506 have occurred before opening another door 4700, 4702. This may allow the transfer chamber 3506 to act as an airlock between the outfeed chamber 4202 and processing chamber 4300 (which may be more stringently controlled).

Once in the outfeed chamber 4202, a bag 26 may be manipulated and inspected at one or more station within the outfeed chamber 4202. In the example depicted in FIG. 2, a mix assisting assembly 2500 is included. A bag 26 may be passed from the gantry assembly 4200 to the mix assisting assembly 2500 and displaced according to a mixing profile or motion profile to help ensure the contents of the bag 26 are homogenized. This may also ensure that any soluble solid concentrate in the bag 26 is dissolved into solution. A particulate inspection system 3500 may image or record the bag 26 and the frames may be analyzed by a control system 15 of the system 10 to determine whether certain contents of interest (e.g. particulate) are present in the bag 26.

The gantry assembly 4200 may transport the bag 26 to a marking assembly 3700 within the outfeed compartment 4202 after inspection. The marking assembly 3700 may provide a marking on the bag 26. If the bag 26 is deemed acceptable for consumption, it may be given a marking indicative of this by the marking assembly 3700. A bag 26 may, for instance, be determined acceptable if no prohibited contents of interest are identified by the particulate inspection system 3500 and sensors of the fluid handling assembly 3900 indicate the fluid supplied to the bag 26 met predefined criteria. If the bag 26 is deemed unacceptable, the marking assembly 3700 may mark the bag 26 indicating this to be the case. The gantry assembly 4200 may transfer the bag 26 from the marking assembly 3700 to a receptacle 4120A, B in an outfeed assembly 4100 in the outfeed compartment 4202. Depending on whether the bag 26 is accepted or rejected, the bag 26 may be placed into specific receptacles 4120A, B of an outfeed assembly 4100 (e.g. outfeed drawer 4102). Access to the receptacles 4120A, B (e.g. outfeed drawers 4102) may be controlled such that rejected bags 26 cannot be collected by a user without specialized access credentials. Receptacles 4120A for acceptable bags 26 may be accessed by users presenting a general credential while receptacles 4120B for unacceptable bags 26 may be accessed by users with more specialized credentials. Thus, receptacles 4120B may be referred to herein as access-controlled receptacles. Receptacles 4120A may be referred to herein as general access receptacles 4120A.

The actuation and coordination of the various assemblies, systems, and stations within the system 10 may be orchestrated by a control system 15 including one or more processor. The control system 15 may also perform analysis and processing to ensure that components of the system 10 are behaving in an anticipated manner and that bags 26 are acceptable for consumption. The control system 15 may generate a log of all activities relating to a single bag 26 with data relevant to the production of that bag 26 from various sensors or systems within the system 10. The log may serve as proof that the bag 26 was produced in accordance with prescribed quality criteria. The control system 15 may also generate screens for display on a graphical user interface 6100 of the system 10 and receive user inputs supplied via the graphical user interface 6100 or other user interface components of the system 10. Screens presented on the graphical user interface 6100 may include instructions, graphics or animations showing an individual how to use the system 10, troubleshooting information, warnings, alerts, alarms, system status screens, login screens, order input screens, and outfeed access request screens.

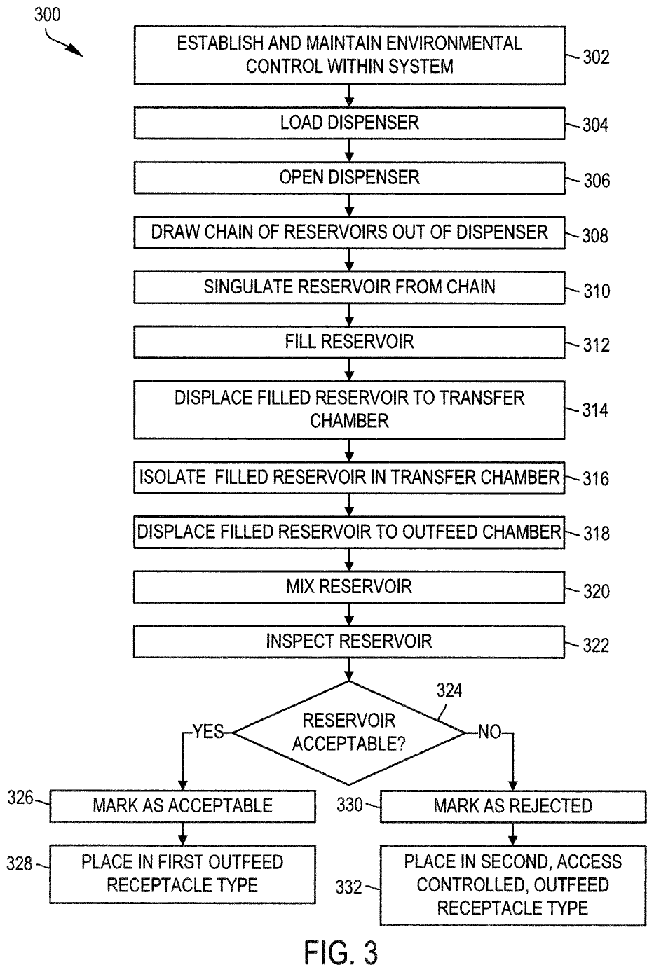

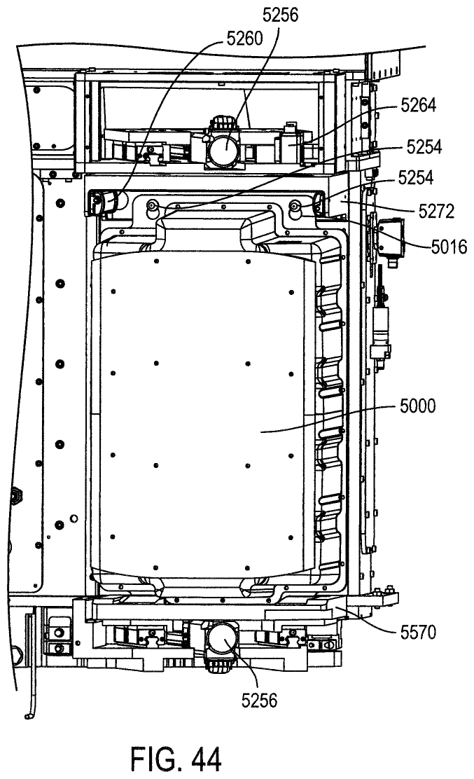

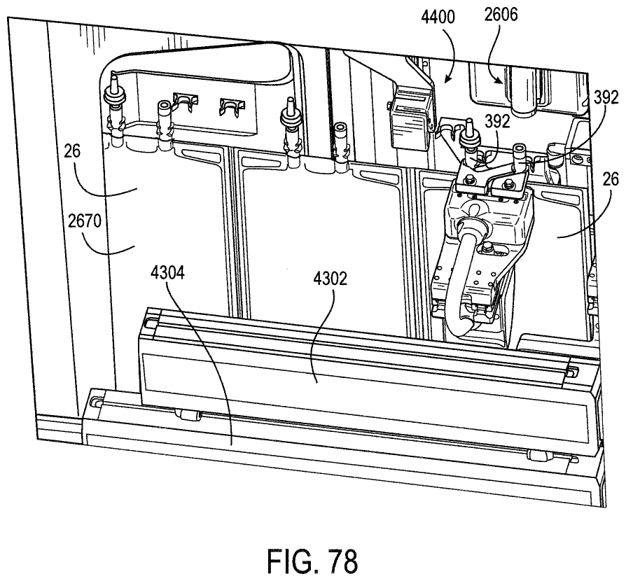

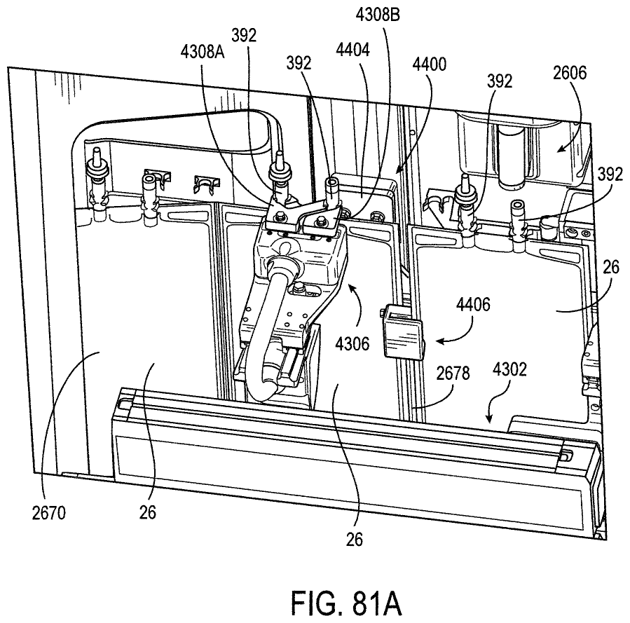

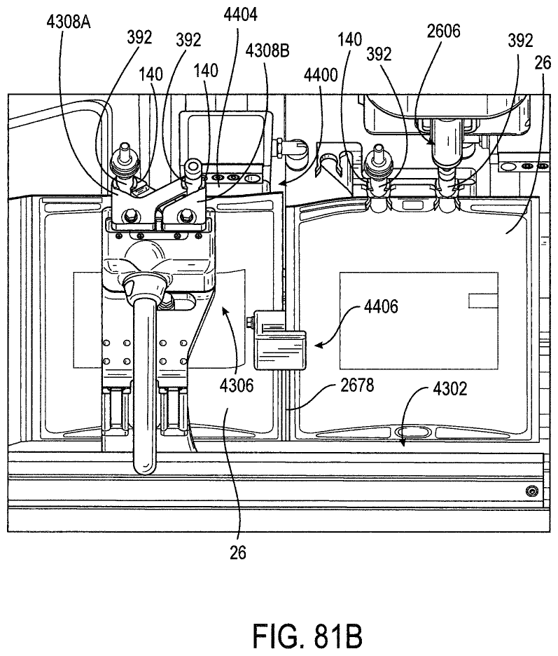

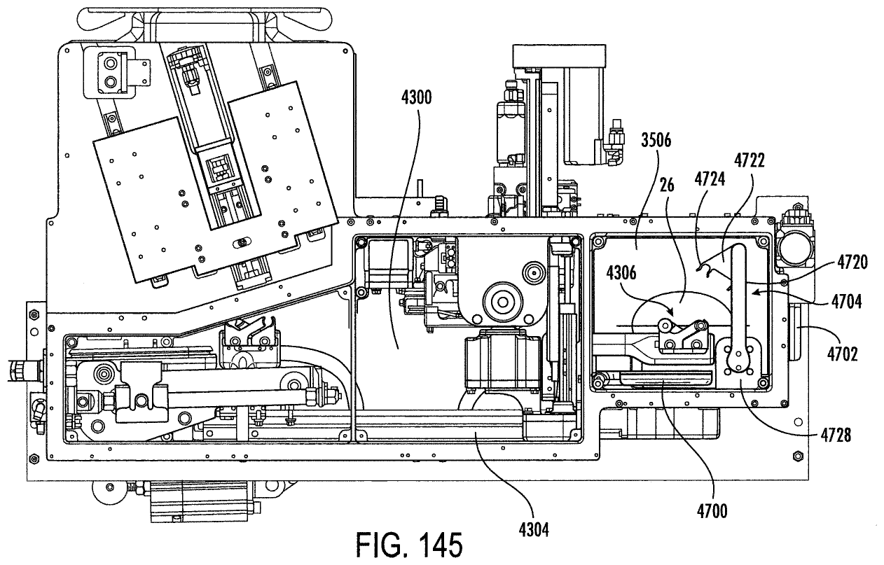

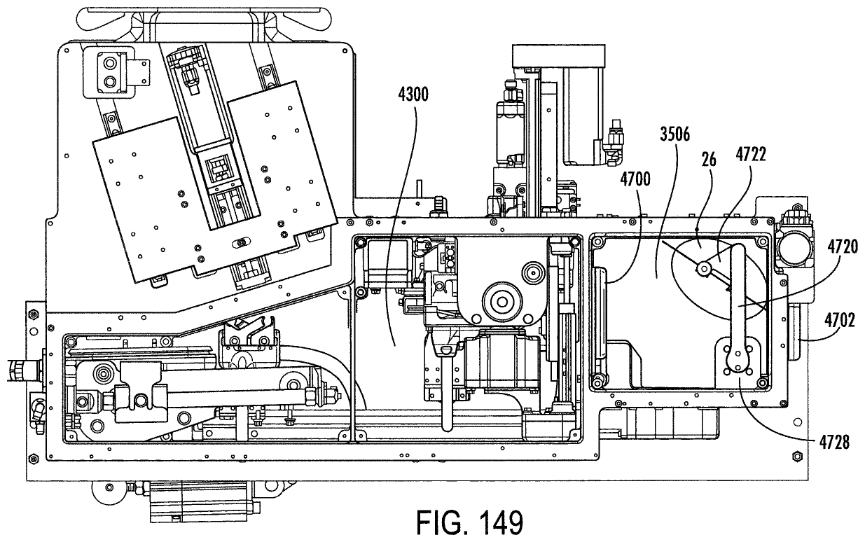

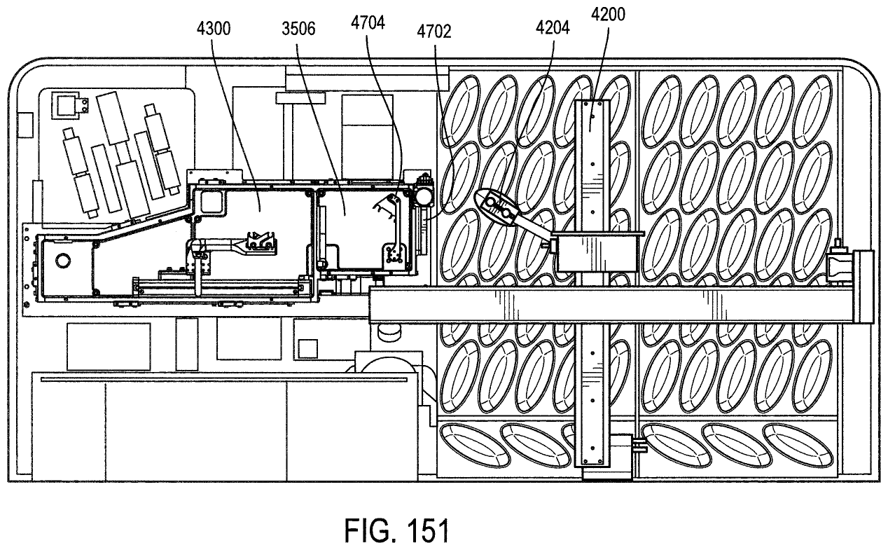

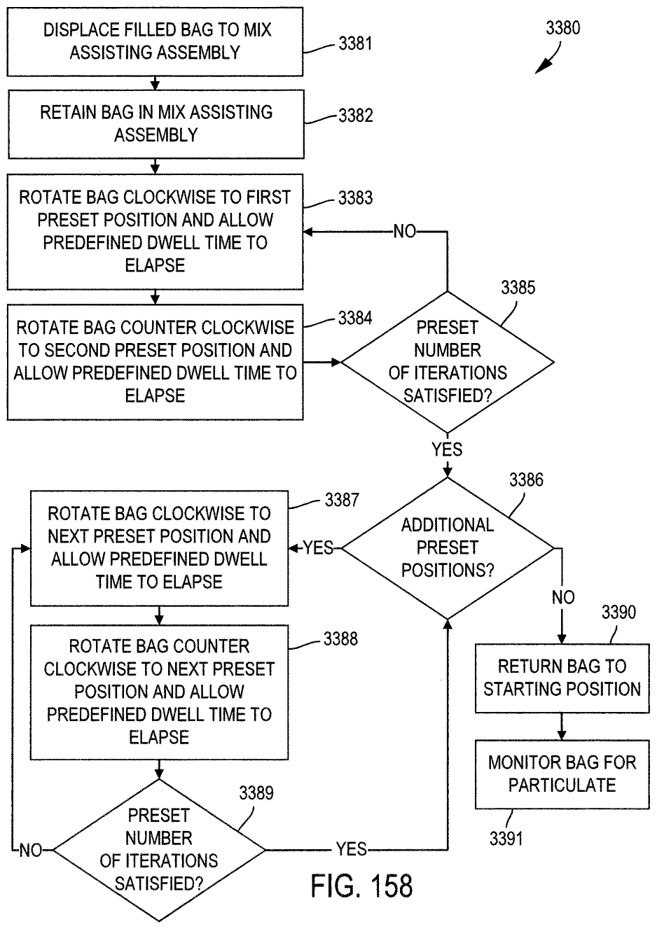

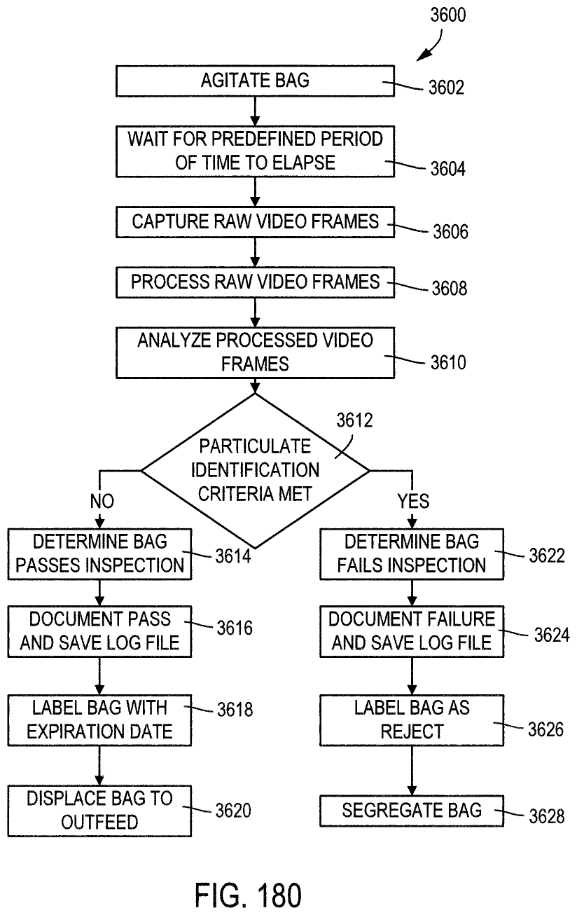

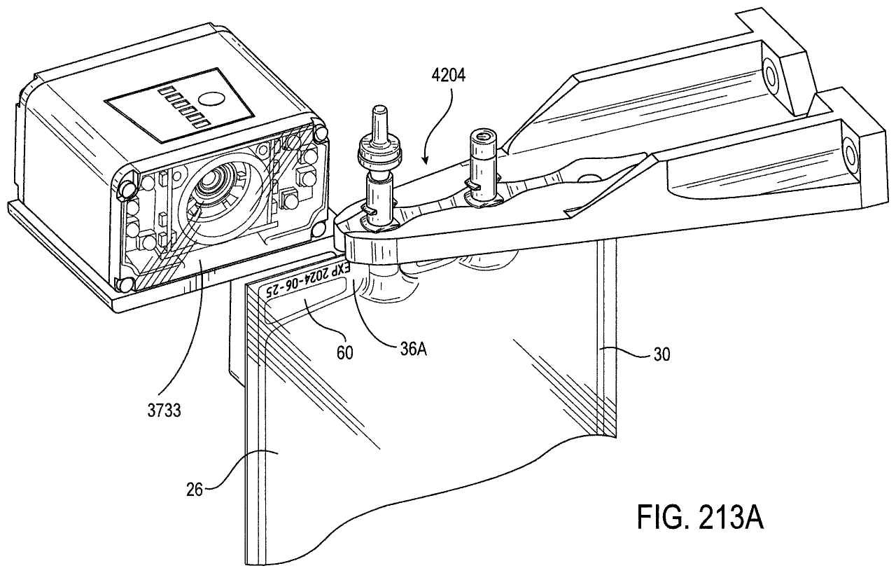

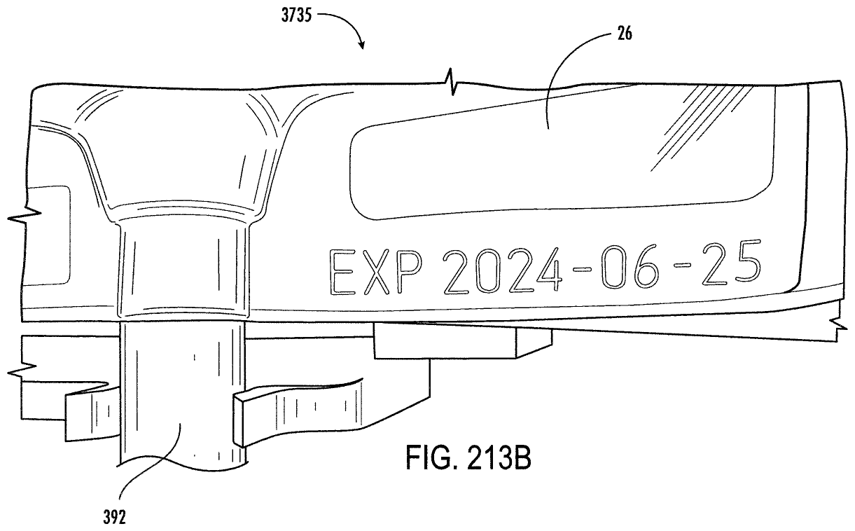

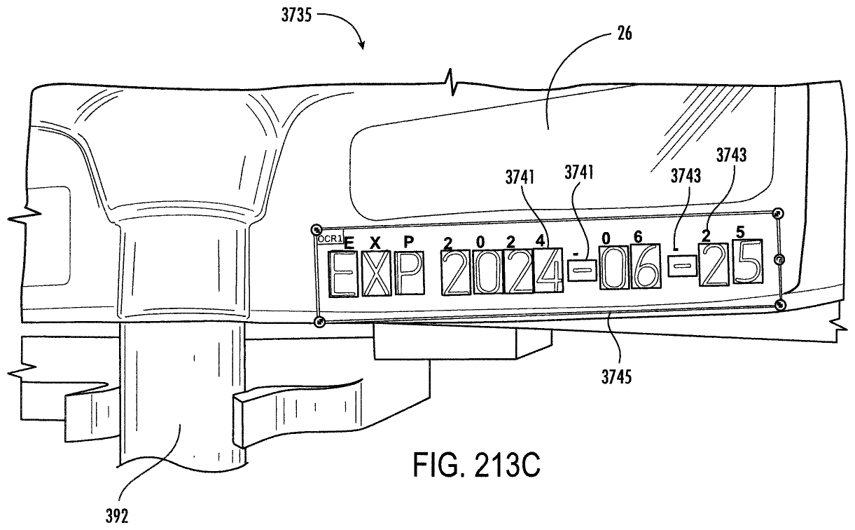

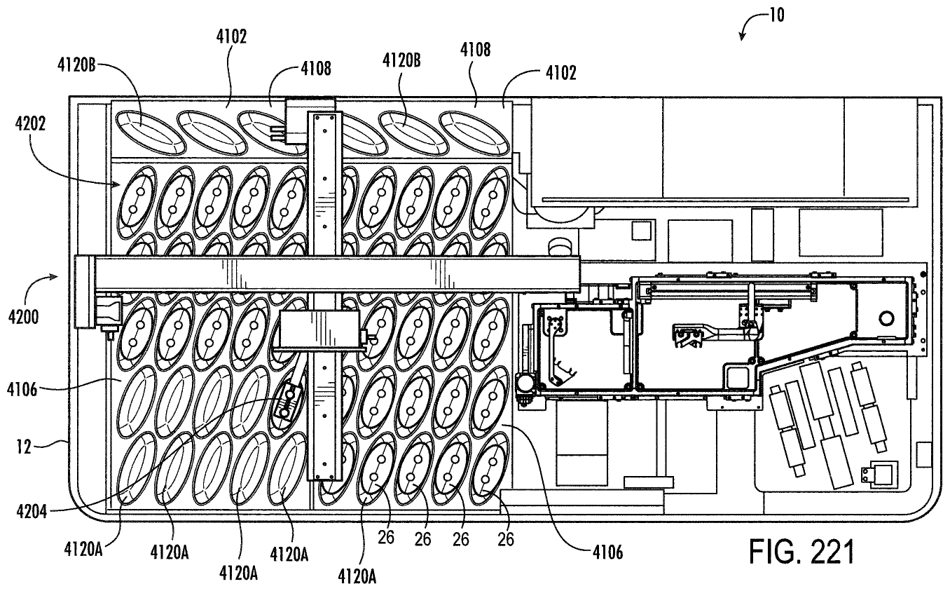

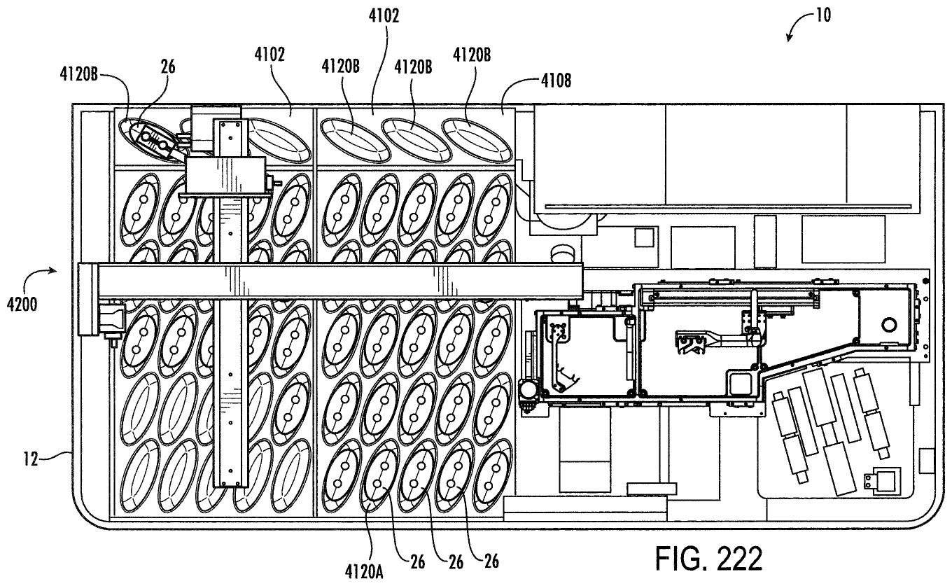

Referring now to FIG. 3, a flowchart 300 is depicted elucidating a number of example actions which may be executed to fill a reservoir with a system 10. In block 302, environmental control within zones of the system 10 may be established and maintained by an air handling assembly 6200 (see, e.g., FIG. 244). A dispenser 5000 may be loaded into a system 10 in block 304 (see, e.g., FIG. 44). In block 306, the dispenser 5000 may be opened via a sanitary interface assembly 5500 of the system 10. A chain 2670 of reservoirs may be drawn out of the dispenser 5000 and into the processing compartment 4300 in block 308 by a bag displacement assembly 2618 of the system 10 (see, e.g. FIG. 78). In block 310, a reservoir may be singulated off the chain 2670 by a singulation assembly 4400 (see, e.g., FIG. 81A). The reservoir may be filled with fluid produced by a fluid handling system 3900 in block 312 (see, e.g. FIG. 139). In block 314, the filled reservoir may be displaced into a transfer chamber 3506 of the system 10 (see, e.g., FIG. 145). The reservoir may be isolated in the transfer chamber 3506 in block 316 (see, e.g., FIG. 149). In block 318, the filled reservoir may be displaced out of the transfer chamber 3506 into an outfeed compartment 4202 (see, e.g., FIG. 151). The contents of the filled reservoir may be mixed at a mix assisting assembly 2500 of the system 10 in block 320 (further described in relation to FIG. 158 for example). In block 322, the reservoir may be inspected for prohibited contents of interest at a particulate inspection system 3500 (further described in relation to FIG. 180 for example). If, in block 324, the reservoir is determined to be acceptable, the reservoir may be marked with a marking assembly 3700 with an indicator that the reservoir is available for use in block 326 (see, e.g., FIG. 213B). The reservoir may then be displaced to a receptacle 4120A in an outfeed assembly 4100 in block 328 (see, e.g., FIG. 221). If, in block 324, the reservoir is determined to be unacceptable, the reservoir may be marked via a marking assembly 3700 with an indication that the reservoir has been rejected in block 330. The rejected reservoir may be deposited in an access controlled receptacle 4120B or an outfeed assembly 4100 in block 332 (see, e.g., FIG. 222).

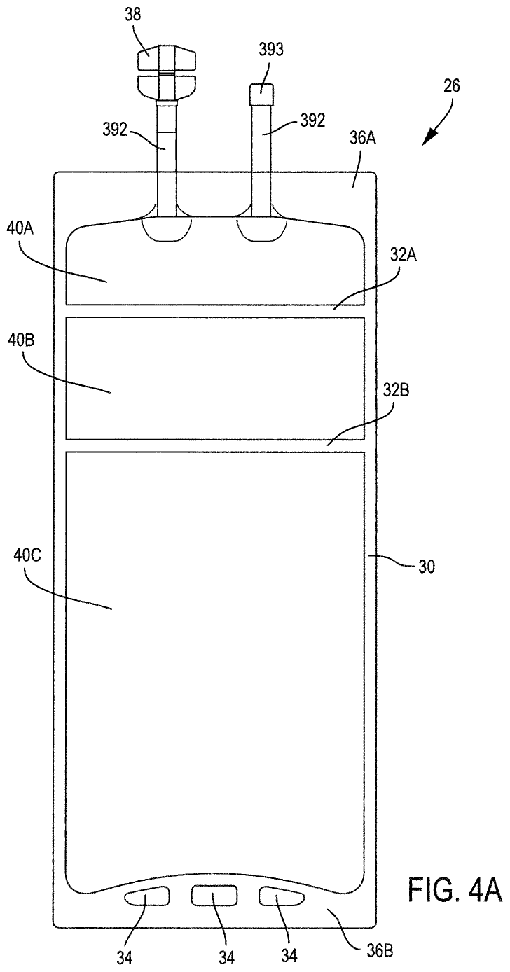

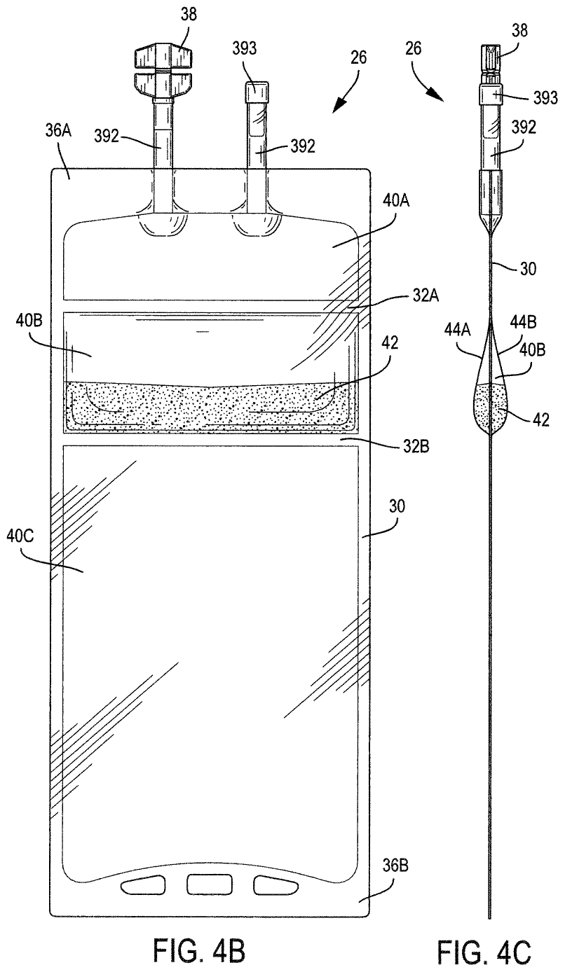

Referring now to FIGS. 4A-C, an exemplary bag 26 is depicted. As shown and as with other bags 26 described herein, the bag 26 may include a first sheet of material 44A and a second sheet of material 44B. The sheets 44A, B may be joined together at a peripheral seal 30 to define at least one interior compartment 40A-C. In alternative embodiments, a single sheet of material could be doubled over and coupled to itself to form the peripheral seal 30. Sheets 44A, B herein may be laminates of a number of different materials. Layers of the laminate may be chosen and ordered to achieve desired objectives. For example, vapor or gas impermeable layer(s) or other barrier layer(s), bonding layer(s), solution compatible layer(s), and reinforcing or durability increasing layer(s) may be included. The materials chosen may be informed by intended sterilization method, weight, optical clarity, durometer, flexibility, heat resistance, elastic modulus, required materials thicknesses, strength, light blocking ability, dielectric/polar properties, etc.

The bag 26 may include at least one port 392 coupled to the sheets 44A, B which establishes a fluid pathway through the peripheral seal 30 to at least one of the interior compartments 40A-C of the bag 26. The peripheral seal 30 may include an enlarged region 36A, B along at least one side of the bag 26. In the example, an enlarged region 36A is present at the port 392 bearing side of the bag 26 and at the side opposing the ports 392. One of the enlarged regions 36A, B may include one or more aperture 34 therethrough which may support hanging of the bag 26 for gravity based administration.

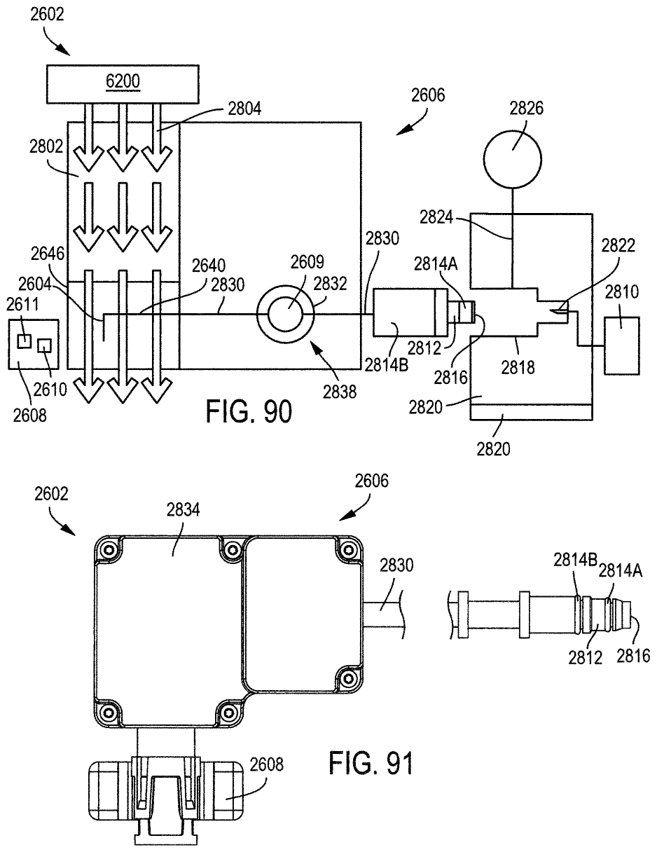

In the example embodiments, the ports 392 include a port 392 with a septum 393 and a port 392 with a stopper 38. The septum 393 may allow access to the interior volume of the bag 26 via a dispensing sharp 2604 (see, e.g., FIG. 139) or needle of a syringe of other filling implement. A filling station 2600 (see, e.g., FIG. 95) may fill the bag 26 via puncturing the septum 393 with a dispensing sharp 2604 of a cartridge 2606 (see, e.g., FIG. 90). The septum 393 may maintain a fluid tight seal and self-close after removal of a dispensing sharp 2604 or needle. The septum 393 may also be used to load additional medical agent into a bag 26 after the bag 26 is filled by a filling station 2600. For example, a bag 26 may be filled at the filling station 2600 to generate a bag 26 of normal saline and a pharmacist may load an antibiotic into a bag 26 by accessing the interior volume of bag 26 via the septum 393 with a syringe needle. The port 392 with the stopper 38 may be accessed by a nurse or other caregiver when the bag 26 is administered. For example, the stopper 38 may be manipulated to reveal a spike receptacle and the bag 26 may be spiked with an administration set. The example stopper 38 is a butterfly type stopper, however, any suitable stopper 38 variety may be used.

The peripheral seal 30 may be formed by welding the sheets 44A, B to one another in the region of the peripheral seal 30. The sheets 44A, B may be robustly coupled at the peripheral seal 30 and may not be separable without destruction of the sheeting 44A, B and bag 26. The sheets 44A, B may also be joined together in various regions to establish the various interior compartments 40A-C within the bag 26. As shown, the bag 26 includes a number of frangible partitions 32A, B. The frangible partitions 32A, B completely isolate the interior compartments 40A-C within the bag 26. The frangible partitions 32A, B may, however, be created in a manner which couples the bag 26 material more weakly than at the peripheral seal 30. The frangible partitions 32A, B may be arranged to separate as fluid is filled into the bag 26.

Referring primarily to FIGS. 4B-C, at least one of the interior compartments 40A-C of a bag 26 may contain a concentrate 42. In the example embodiment, the second interior compartment 40B contains a concentrate 42. In some specific examples, each bag 26 may include at least one compartment 40A-C filled with at least one crystalline salt (e.g. sodium chloride) which is sufficient to generate a solution of desired concentration (e.g. 0.9% normal saline or 0.45% half normal saline) when the bag 26 is filled with a prescribed volume of fluid. Sugars, and sugar salt mixtures may also be used in some examples, though any desired active pharmaceutical ingredient(s) may be included. Concentrates may include concentrates for Ringer's solution, Lactated Ringer's solution, Hartmann's solution, sugar solutions (e.g. D5W), sugar saline solutions (e.g. D5NS, ⅔ D5W & ⅓ NS). In other embodiments, the concentrate 42 could, for example, be dialysate precursors for hemodialysis or peritoneal dialysis. Different components of a concentrate 42 may be in different compartments 40A-C. The concentrate 42 may at least partially fill the compartment 40A-C in which it is disposed. In general, embodiments are described herein in relation to normal saline though this is merely exemplary. In various embodiments described herein, any concentrate 42 described herein may be included in any bag 26 described or shown herein and the concentrate included may be sufficient to produce a bag 26 with any desired concentration when filled (e.g. hypotonic, isotonic, or hypertonic). Any compartments of the bag 26 in which concentrate 42 is absent may be empty or devoid of contents and in a collapsed state. There may be a miniscule amount of gas (which may be kept as small as is practicable) in the non-concentrate containing compartments while still considering these compartments empty. A vacuum could also be pulled on these compartments to render them empty. This may ensure that the bag 26 has maximum packing density. It may also prevent the presence of any substantial air volume within the bag 26 when filled.

Still referring primarily to FIGS. 4B-C, isolating the concentrate 42 within a compartment 40B which is intermediate two compartments 40A, C may be particularly desirable for bags 26 intended to be filled on site shortly prior to use. It may be further desirable for bags 26 in which the concentrate is a lyophilized, powder, or a crystalline solid. The partition 32A between the first compartment 40A and the second compartment 40B inhibits displacement of concentrate 42 into the ports 392 of the bag 26. This arrangement blocks concentrate 42 from becoming stuck in a port 392 and simplifies ensuring that all concentrate 42 has homogenously mixed within the bag 26. The first partition 32A may be placed as close to the ports 392 as is practicable. For example, the first partition 32A may be placed at a location which is between 5-30% of the length of the bag 26 from side of the peripheral seal through which the ports 392 extend.

Bags 26 may typically be filled while oriented with the axes of the ports 392 extending in the vertical direction (parallel to the direction of acceleration due gravity). As the first compartment 40A of the bag 26 is filled with fluid from a filling station 2600, the first frangible partition 32A may be defeated. The fluid may drop into the now accessible second compartment 40B causing turbulence and mixing of the fluid with the concentrate 42. The second compartment 40B may be dimensioned to have an interior volume larger than the volume of concentrate 42 contained therein. Thus, the extra volume may act as a spacer which permits the fluid in the first compartment 40A to fall some distance before contacting the concentrate 42. As the joined volume of the first and second compartment 40A, B is filled, the second frangible partition 32B may eventually be defeated. The contents of the bag 26 may again drop into the now accessible third compartment 40C resulting in further mixing. The rounded corners and curved wall at the end of the bag 26 opposite the ports 392 may augment the mixing as the contents of the bag 26 drop into the third compartment 40C. The bag 26 may subsequently be filled to a predetermined amount. The amount of fluid dispensed into the bag 26 by the filling station 2600 may be appropriate to generate a fluid with a desired concentration given the concentrate 42 initially provided in the bag 26. In order for a bag 26 to accept the full volume of fluid dispensed into the bag 26 by the filling station 2600, all partitions 32A, B of the bag 26 may be defeated. This may ensure that the bag 26 does not include partitions 32A, B which a user must remember to disrupt before use reducing potential for administration errors.

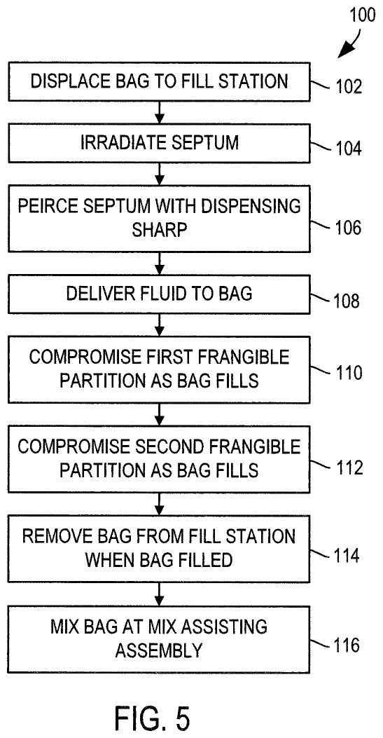

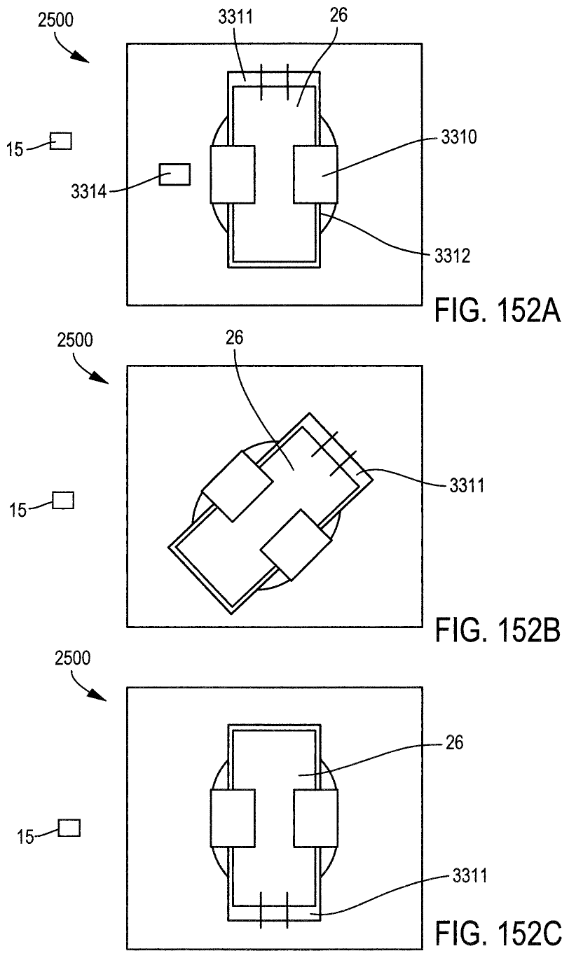

Referring now to FIG. 5, a flowchart 100 is depicted detailing a number of example actions which may be executed to fill a bag 26 of the type shown in FIGS. 4A-C. As shown, a bag 26 may be displaced to a filling station 2600 in block 102. The septum 393 of the bag 26 may be irradiated, in block 104, by an irradiation assembly 2608 (further described in relation to, e.g., FIG. 95). The septum 393 of the bag 26 may be pierced with a dispensing sharp 2604 of a cartridge 2606 (see, e.g., FIG. 95) of the filling station 2600 in block 106. In block 108, fluid may be delivered to the bag 26. As the bag 26 is filled with fluid, the first compartment 40A may reach capacity and the pressure of the fluid may compromise the first frangible partition 32A in block 110. In block 112, the combined volume of the first and second compartments 40A, B may reach capacity and the pressure of the fluid may compromise the second frangible partition 32B. The bag 26 may be removed from the filling station 2600 in block 114 once the bag 26 has been filled a desired amount. The bag 26 may then be displaced to a mix assisting assembly 2500 (further described in relation to, e.g., FIGS. 152A-C) and the contents of the bag 26 may be mixed in block 116. The mixed bag 26 may have substantially homogenous content and all solid concentrate 42 may be dissolved into solution.

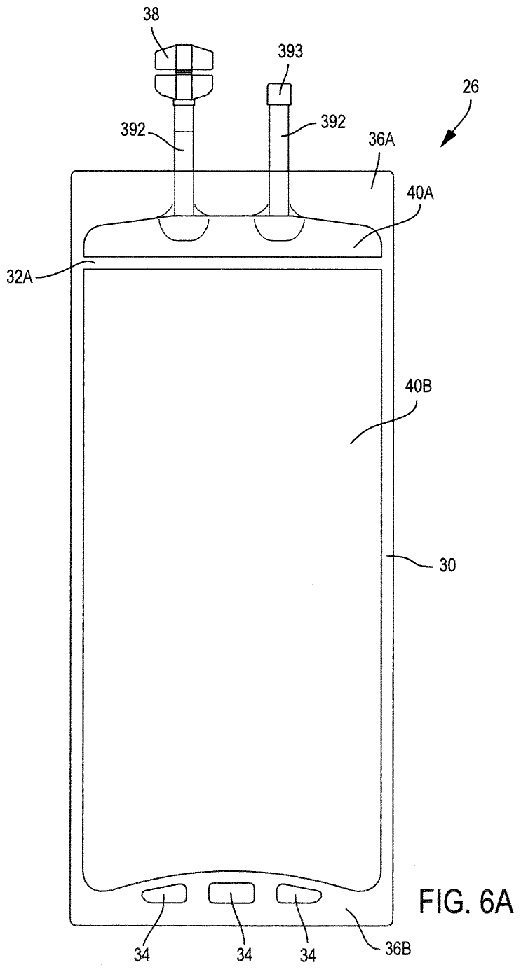

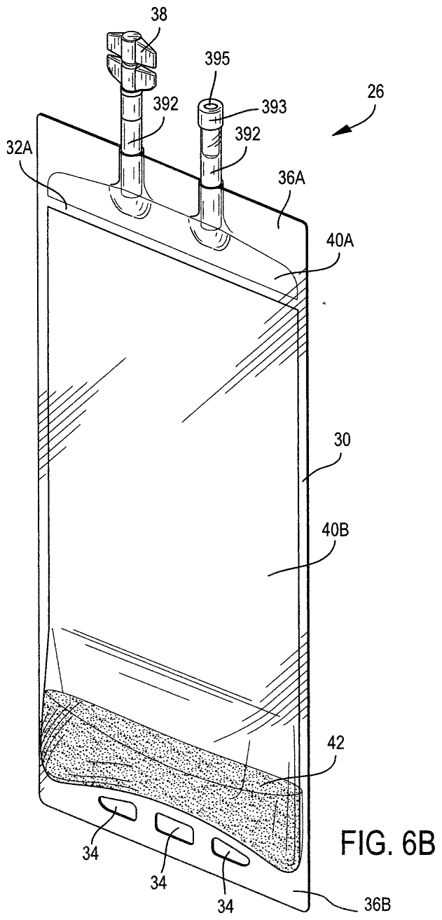

Referring now to FIGS. 6A-B, another example bag 26 with a first and second compartment 40A, B is depicted. The frangible partition 32A of the example bag 26 is placed proximate the ports 392. For example, the first partition 32A may be placed at a location which is between 5-25% of the length of the bag 26 from side of the peripheral seal through which the ports 392 extend. This may ensure that the ports 392 are segregated from any concentrate 42 in the second compartment 40B of the bag 26. The first compartment 40A may be empty, and the second compartment 40B may contain substantially only concentrate 42. The bag 26 may be filled as described in relation to FIG. 5, however, block 112 would be omitted.

As the bag 26 is filled, the first compartment 40A may reach capacity and the frangible partition 32A may be compromised as additional fluid is dispensed into the bag 26. The fluid in the first compartment 40A may drop into contact with the fluid in the second compartment 40B generating turbulence and encouraging mixing. After the frangible partition 32A is defeated, fluid dispensed into the bag 26 may fall nearly the length of the bag 26 to the solution (and yet to be dissolved concentrate 42) at the opposing side of the bag 26. This may help to encourage more vigorous mixing as the bag 26 is filled. As described in relation to FIG. 5, the bag 26 may be mixed at a mix assisting assembly 2500 (see, e.g., FIGS. 152A-C) after it is filled with the desired amount of fluid by a filling station 2600 (see, e.g., FIG. 95).

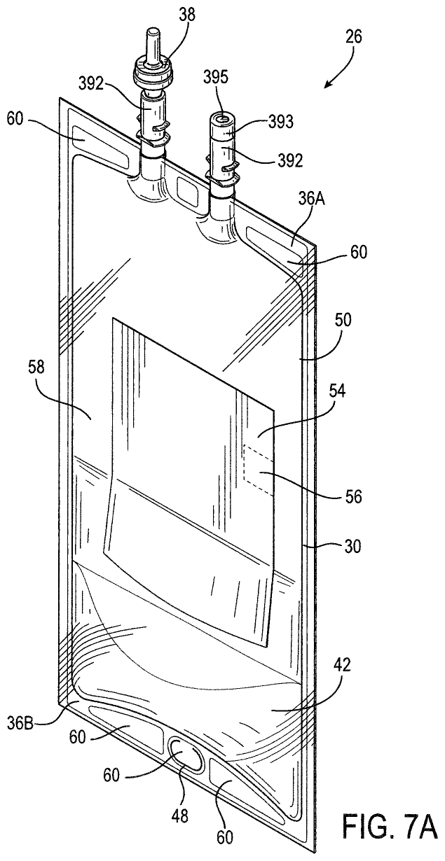

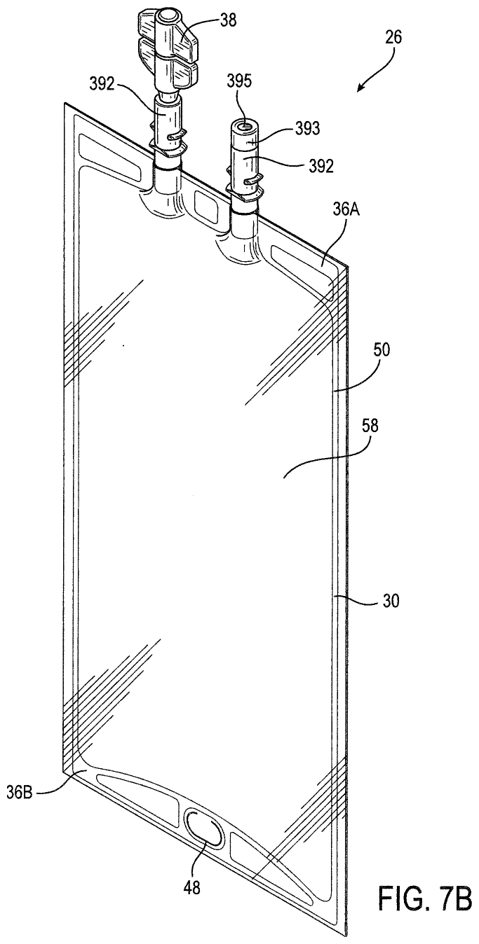

Referring now to FIGS. 7A-B, further exemplary bags 26 are depicted. In some embodiments, or for some concentrates 42, it may be desirable to have a single interior compartment 58 within the bag 26. As shown, the bag 26 may be filled with a liquid concentrate (e.g. high molarity solution or salt brine). In some specific examples an at least five molar solution of sodium chloride may be used. The stopper 38 of the bag 26 in FIG. 7A is a tulip type stopper 38. Such stoppers 28 may be used in any other bag 26 shown or described herein.

As shown, the bags 26 in FIGS. 7A-B include an enlarged region 36A of the peripheral seal 30 at the side of the bag 26 through which the ports 392 extend. The opposing side of the bag 26 also includes an enlarged region 36B of the peripheral seal 30. These regions 36A, B may include uncoupled areas 60 where the sheeting 44A, B (see, e.g., FIG. 4C) is not welded together. A slit 48 may also be included in the enlarged region 36B or an uncoupled area 60 therein. The slit 48 may allow a hanger to pass through the bag 26 in this region to facilitate gravity based administration.

Though this may be true of any bag shown or described herein, it may be preferable that sheets 44A, B of bags 26 loaded with a liquid concentrate may each be a ply of a multiple ply stock. Two-ply layers (e.g. adjacent lamina layers of the respective plies) may be separable to form a fillable interior volume which is defined by the peripheral seal 30. The sheets 44A, B may be plies of PolyCine APP-114-PB material in certain embodiments.

In some examples of bags 26 containing liquid concentrate, the liquid concentrate 42 may be placed in a compartment of the bag 26 which is segregated from at least one other compartment. A frangible partition may be created separating the liquid concentrates from the ports 392 for example. This may ensure that concentrate 42 does not become entrapped within the ports 392. In such embodiments, the concentrate 42 may be isolated within a compartment having a volume slightly larger (1-10%) than the concentrate 42 volume. This may limit the surface area in contact with any possible gas in the compartment minimizing evaporation of solvent through the bag 26. In some embodiments, the liquid concentrate 42 may be isolated in a compartment at a side of the bag 26 opposite the ports 392. Alternatively, the liquid concentrate 42 may be compartmented into an intermediate portion of the interior volume of the bag 26. The liquid concentrate 42 could, for example, be placed in a compartment similar to compartment 40B of FIG. 4B.

Any of the bags 26 described herein may include a label 54. The label 54 may be adhered to the bag 26 or printed directly to the bag 26. Any of the bags 26 described herein may include at least one indicium 56 which is machine-readable. Such indicia 56 may include barcodes, QR codes, GS1 codes, data matrices, bokodes, RFID tag, NFC tag, etc. The indicia 56 may include various information about the bag 26. For example, the indicia 56 may include a unique bag 26 identity, a lot identity, production line identity, date of manufacture, an expiration or use by date, information defining the concentrate in the bag 26, a mass or volume of concentrate or brine provided to the bag 26 during manufacture, bag 26 capacity information, etc.

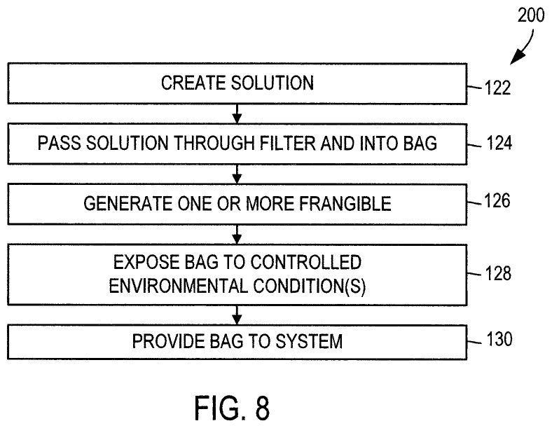

Referring now to FIG. 8, a flowchart 120 is depicted showing a number of example actions which may be executed to prepare a bag 26 before providing it to a system 10 for filling. In some examples, a bag 26 may be filled with a liquid concentrate 42 and a component of the concentrate 42 may be at least partially removed from the sealed bag 26. For example, the solvent component of the concentrate 42 may be evaporated from the bag 26. Using the example of a salt brine, the bag 26 may be formed of sheeting 44A, B (see, e.g. FIG. 4C) which displays some permeability to water vapor. Any biocompatible sheeting permeable to water vapor may be used. At least some of the water content in the bag 26 may be evaporated out to dehydrate the bag 26. This may lower the weight and volume of the bag 26 which may allow the bag 26 to be more easily handled. It may also make shipping of the bag 26 simpler. For example, concerns relating to shipping a corrosive liquid brine could be mitigated by evaporating the water component of the brine through the sheeting 44A, B before shipping. Evaporation may also allow for more bags 26 to be placed in a dispenser 5000 of the same size. It may further simplify spooling or packing of the bags 26 into the dispenser 5000. The at least partial evaporation would make the already osmotically challenging interior of the bag 26 yet more osmotically challenging for microbial growth. Additionally, the variability between lots of bags 26 would be minimized and better tracked as the majority of evaporation (or all) through the bag 26 sheeting 44A, B would be controlled and may be subject to monitoring.

As shown, a solution may be formed in block 122. The solution may be a solution of a desired molarity (e.g. 5 Molar or greater saline) or a saturated solution. The solution may be made from water for injection quality water and a pharmaceutical grade solid agent. In some embodiments, the solution may be a super saturated solution. In some embodiments, the solution may also be a high temperature solution. For example, a sugar solution may be created and maintained substantially above ambient (e.g. 80° C. or greater) in order to dissolve more of a desired ingredient into the concentrate 42. This may allow the total volume of concentrate 42 loaded into the bag 26 to be kept as low as is practicable. Note the above may be done in any bag 26 herein including a liquid concentrate 42 in order to lower the overall weight and volume of the bag 26. It is not necessary that solvent be subsequently removed from the bag 26 as described in relation to flowchart 120 in FIG. 8.

In block 124, the solution may be passed through a filter 124 and into the bag 26. The amount of solution dispensed into the bag 26 may be dependent on the desired concentration of solution intended to be filled into the bag 26 once filled by the system 10. Concentration of the solution may be monitored or otherwise determined. Temperature of the solution and volume of the solution dispensed into the bag 26 may be used to determine the mass of solute transferred into the bag 26. Metering of fluid into the bag 26 may be controlled to ensure a desired mass of solute is dispensed into the bag 26. Certain solid crystalline concentrates (even those for pharmaceutical purposes) may not be available in a particulate free from due to the manner in which they are produced. The filter may ensure that the solution supplied to the bag 26 is particulate free.

In block 126, at least one frangible partition 32A may be generated in the bag 26. The partition may be formed proximate the ports 392 making a small compartment 40A within the bag 26. This may ensure that any crystallized solute does not become lodged in the ports 392 prior to use. In alternative embodiments, no frangible may be formed and block 126 is optional.

The bag 26 may then be exposed to controlled environmental conditions which facilitate removal of the solvent from the bag 26 in block 128. For example, the controlled environment may be an elevated temperature environment. The temperature may be held in a defined temperature range. This range may ensure that the bag 26 is subjected to an environment at a temperature of 50-60° or higher, for instance. The controlled environment may alternatively or additionally be held within a predetermined humidity range. For example, the controlled environment may be maintained at a humidity of 5-10% or lower. This may facilitate removal of solvent from the bag 26 through the sheeting 44A, B of the bag 26.

In alternative embodiments, at least one of the ports 392 for the bag 26 may provide a fluid communication path from the interior of the bag 26 to the controlled environment during dehydration. The controlled environment may be controlled to a desired clean room standard in such examples (with the temperature and humidity being as described above. The ports 392 would be subsequently sealed or stopped (e.g. with a septum 393) after dehydration.

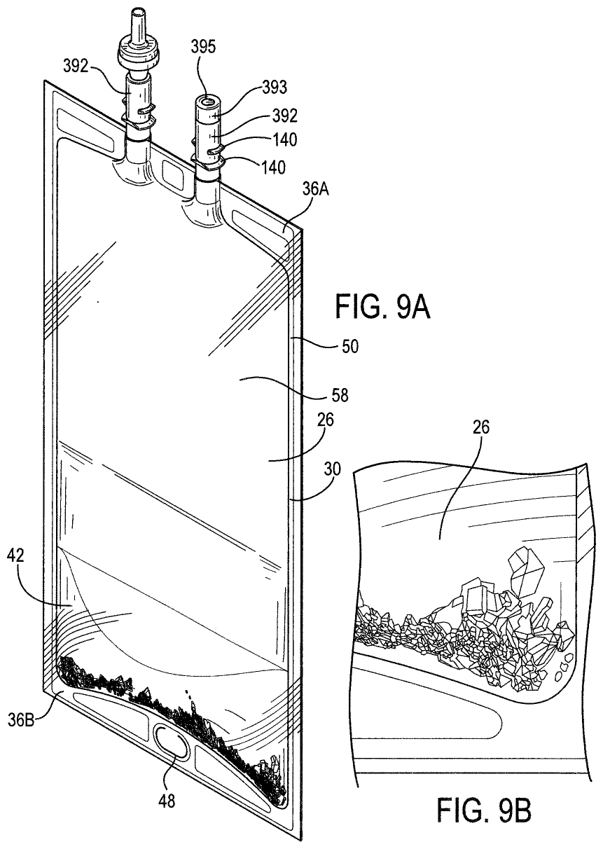

In some embodiments, the bag 26 may be substantially completely dehydrated. Alternatively, least a majority of the original liquid in the bag 26 may be removed. Some solutes may fall out of solution as the solvent is evaporated through the sheeting 44A, B, material. The bag 26 may preferably be oriented vertically with the ports 392 superior to the opposing end of the bag 26. This may ensure any recrystallization does not occur in the ports 392. Thus, a bag 26 filled with filtered, particulate free liquid concentrate may be processed to generate a bag 26 containing an at least partially solid concentrate. An illustration of the example bag 26 depicted in FIG. 7A containing a partially solid concentrate is depicted in FIG. 9A. An illustration of a region of an example bag 26 containing a partially solid concentrate is shown in FIG. 9B.

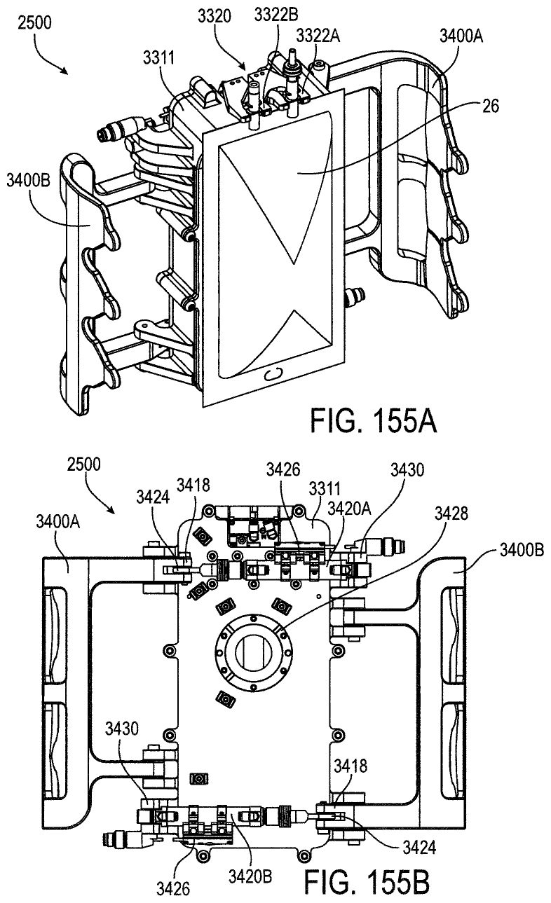

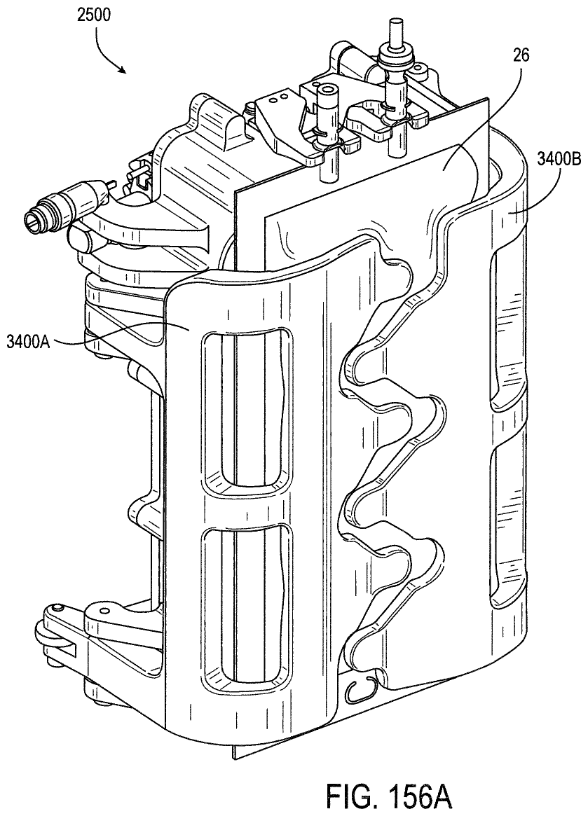

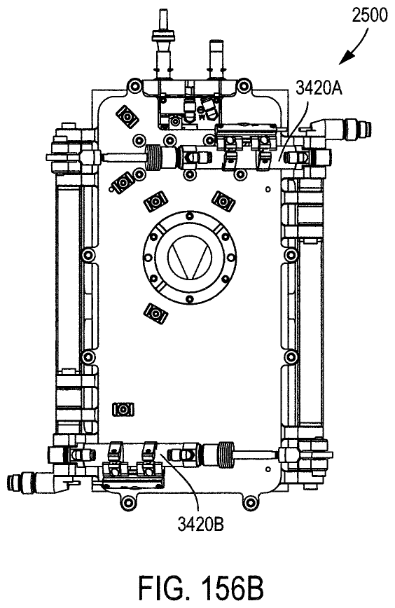

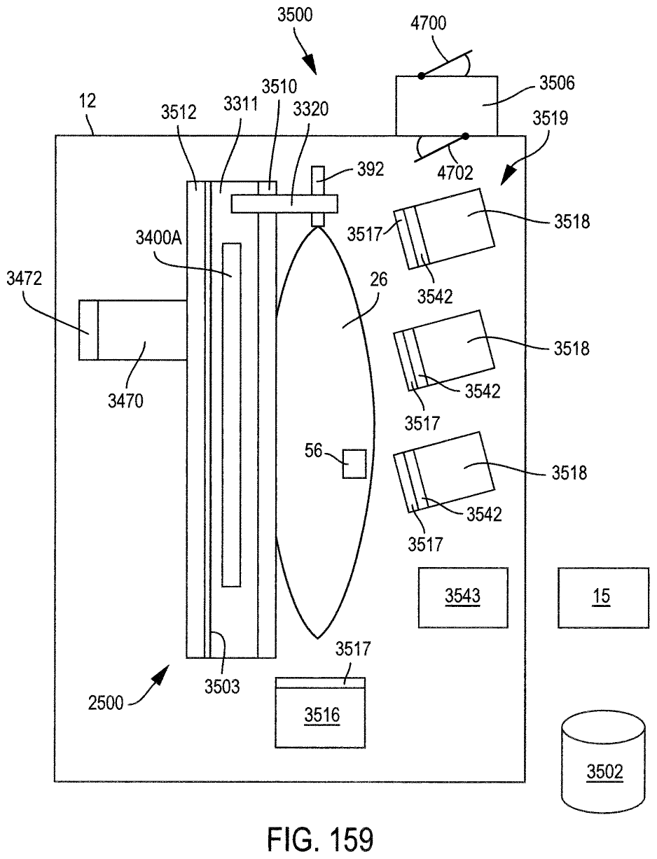

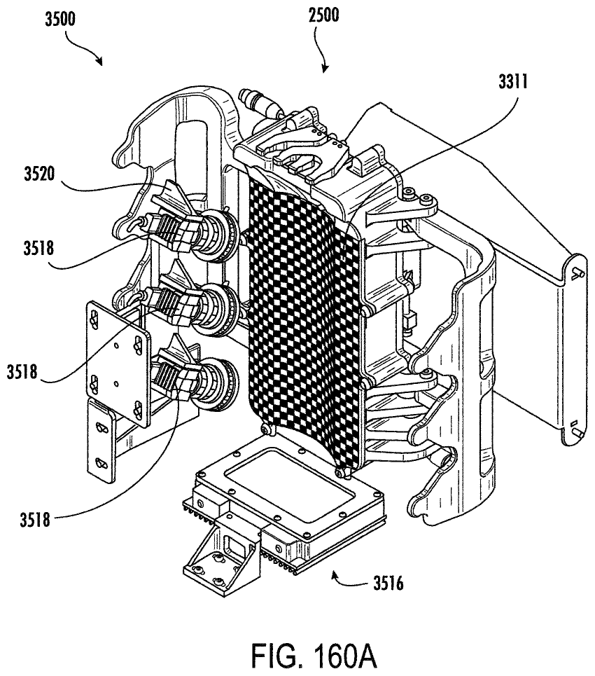

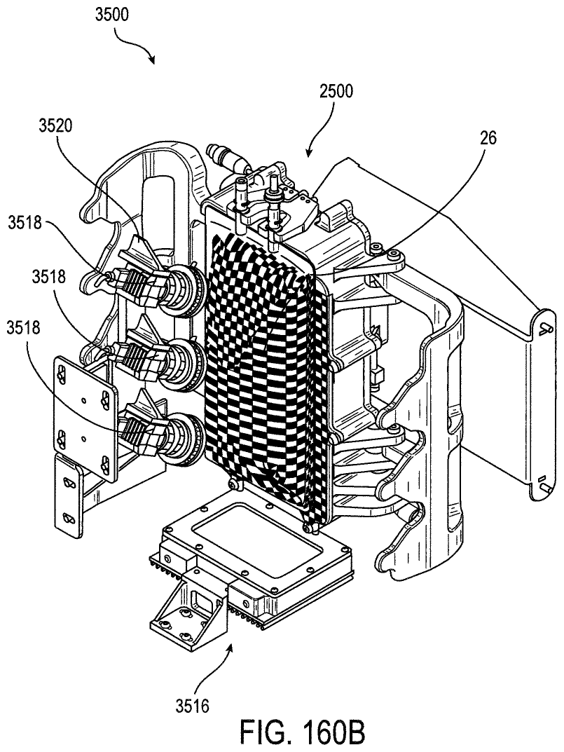

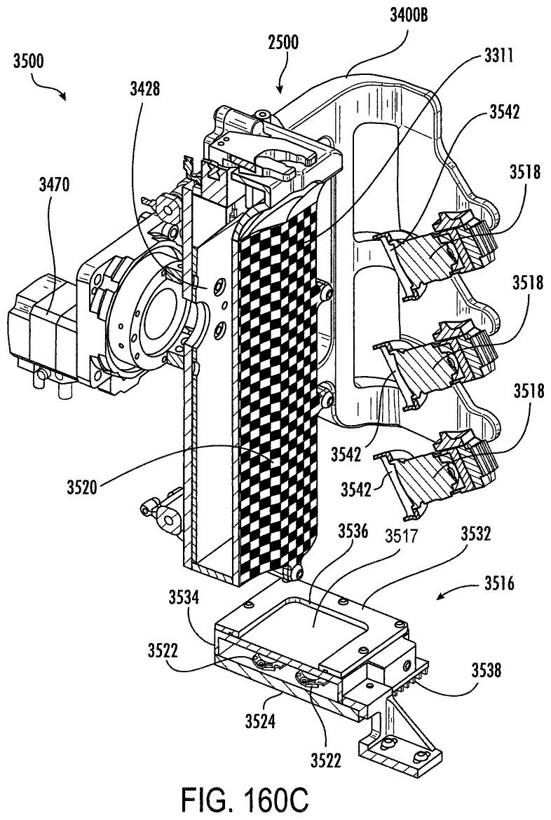

The bag 26 may be provided to a system 10 in block 130. The bag 26 may be subjected to vigorous mixing in a mix assisting assembly 2500 after filling (further described in relation to FIGS. 155A-156B). The bag 26 may also be monitored for undissolved crystallized concentrate in a particulate inspect system 3500 (further described in relation to FIGS. 159-160C). In the event undissolved crystalline concentrate is detected or if contents other than air are detected in the bag 26, the bag 26 may be subjected to further mixing in a mix assisting assembly 2500. The bag 26 may be re-inspected with a particulate inspection system 3500 to confirm the concentrate 42 is fully dissolved. In some embodiments, the bag 26 may be subjected to further mixing and inspection up to a predetermined cap on the number of retries.

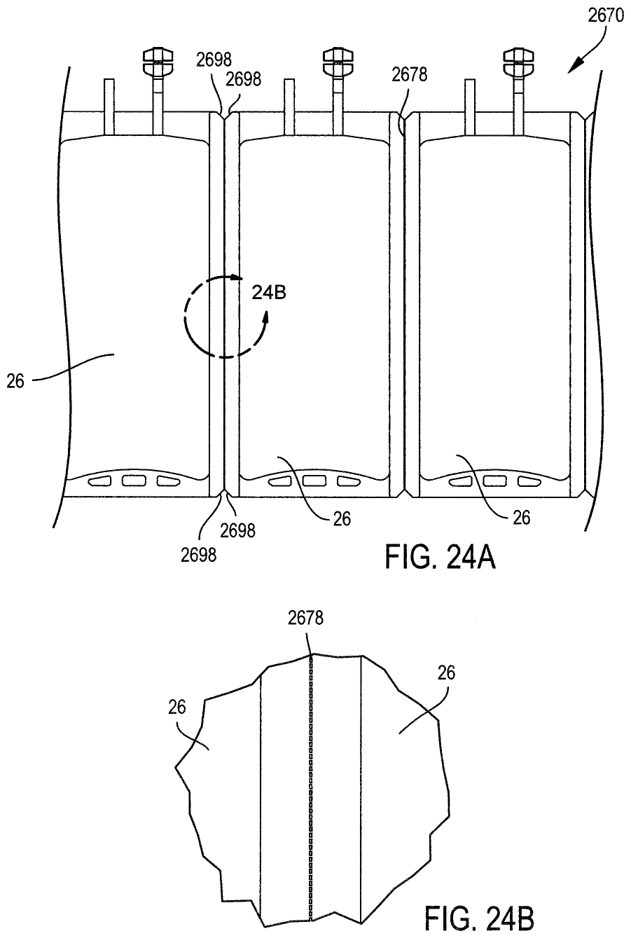

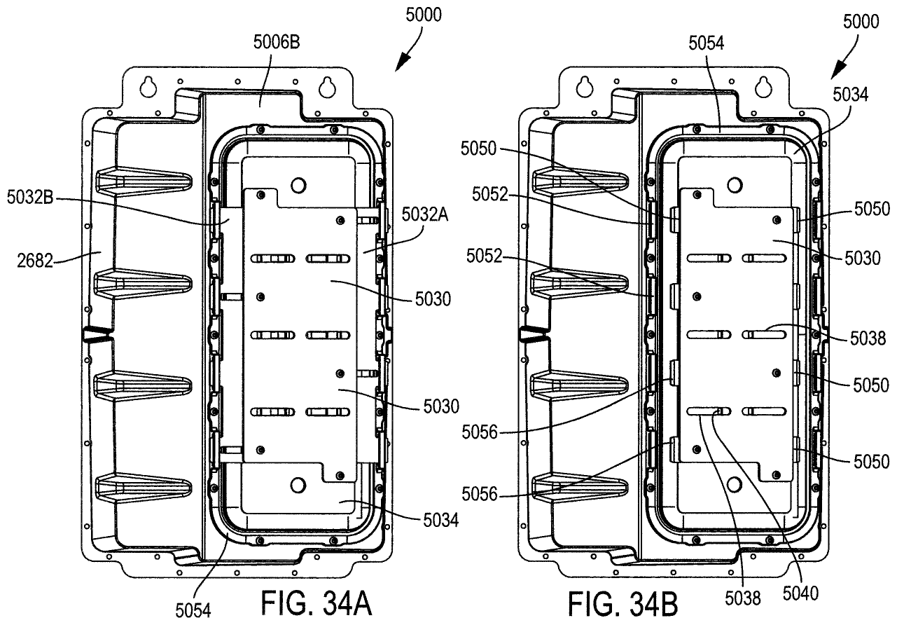

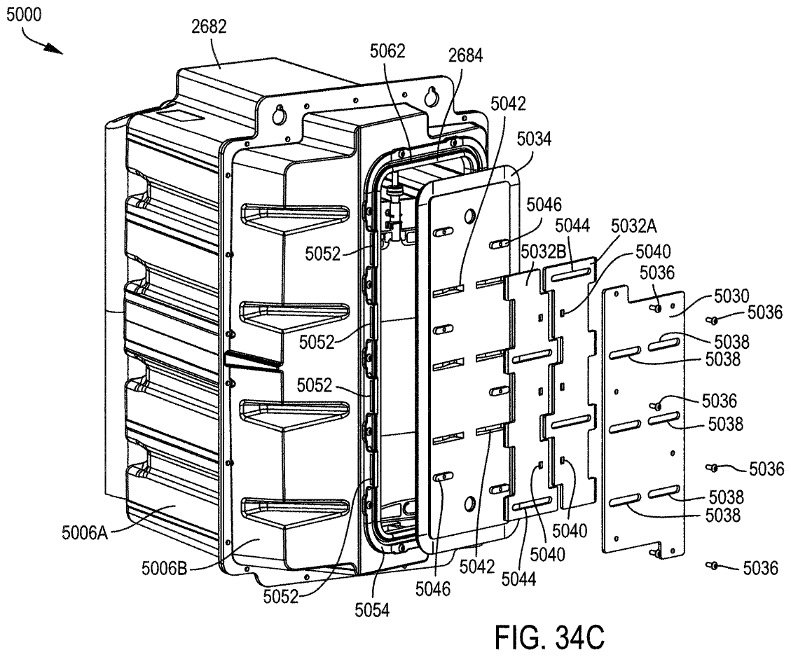

Though the flowchart 120 of FIG. 8 is described in the context of single bags 26, is should be understood that an entire chain 2670 (see, e.g., FIG. 24A) of bags 26 may be used. Each of the individual bags 26 would be filled as described in relation block 124. The entire chain 2670 of bags 26 would then be placed in the controlled environment described in relation to block 128. An entire dispenser 5000 (see, e.g., FIG. 32) loaded with a chain 2670 of bags 26 may be placed in a controlled environment as well. The removable cover assembly 5002 (see, e.g., FIGS. 34A-C) may, for example, be absent or removed to allow communication between the interior of the dispenser 5000 and the controlled environment.

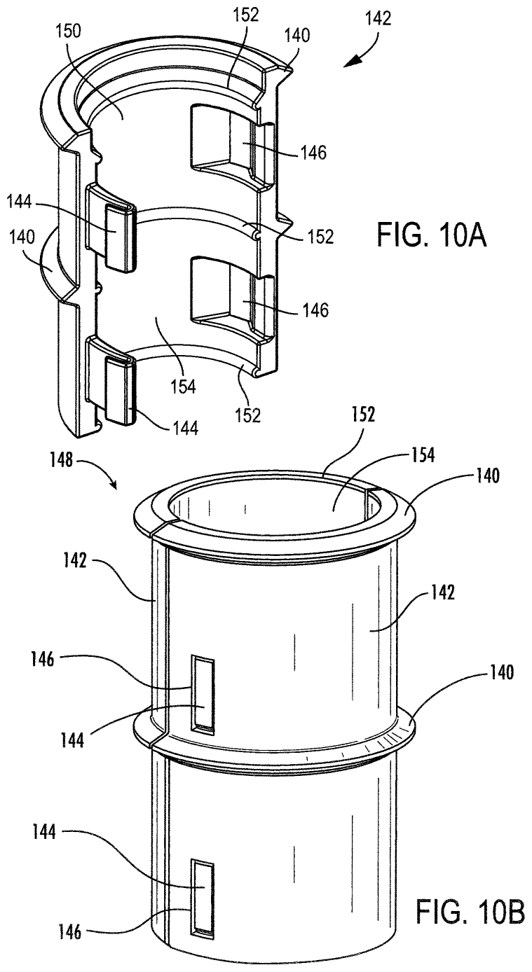

Referring now to FIGS. 10A-B, the ports 392 of a bag 26 may include a set of projections which extend outwardly from the ports 392. The projections may generally extend in a direction perpendicular to the axis of the port 392 to which they belong. Each projection may at least partially surround the port 392 from which it extends. Though a variety of projection types may be used, the example embodiment shown in FIGS. 10A-B includes ribs 140. The projections may assist in retaining a bag 26 with automation equipment such as a grasper 160 (see, e.g., FIG. 12). Jaws 162 (see, e.g., FIG. 12) of the grasper 160 may be adjacent a projection on the port 392. For example, the jaws 162 could close on a port 192 in a location between a set of projections on the port 392. The projections may overhang the grasper jaws 162 limiting the displacement of the bag 26 relative to the grasper 160 in the direction parallel to the axis of the port 392. Projections may be spaced apart a distance slightly (5-10%) greater than the thickness of the grasper jaw 162.

Still referring to FIGS. 10A-B, the projections may, in some examples, be coupled to the ports 392. As shown, a set of projection bearing bodies 142 may be included. The projection bearing bodies 142 may each have at least one clip projection 144 and at least one retention catch 146 (best shown in FIG. 10A). The clip projections 144 of one projection bearing body 142 may be advanced into the retention catches 146 of a cooperating projection bearing body 142 to form a ribbed sleeve 148 as shown in FIG. 10B. The projection bearing bodies 142 may snap fit together and the ribs 140 on each may align to form a single rib 140 which extends around the sleeve 148. In the example embodiment, the ribbed sleeve 148 is substantially cylindrical and the projection bearing bodies 142 include arced walls 150 which extend across a substantially 180° arc. Any suitable sleeve 148 shape may be used so long as a port 392 may be accommodated therein.

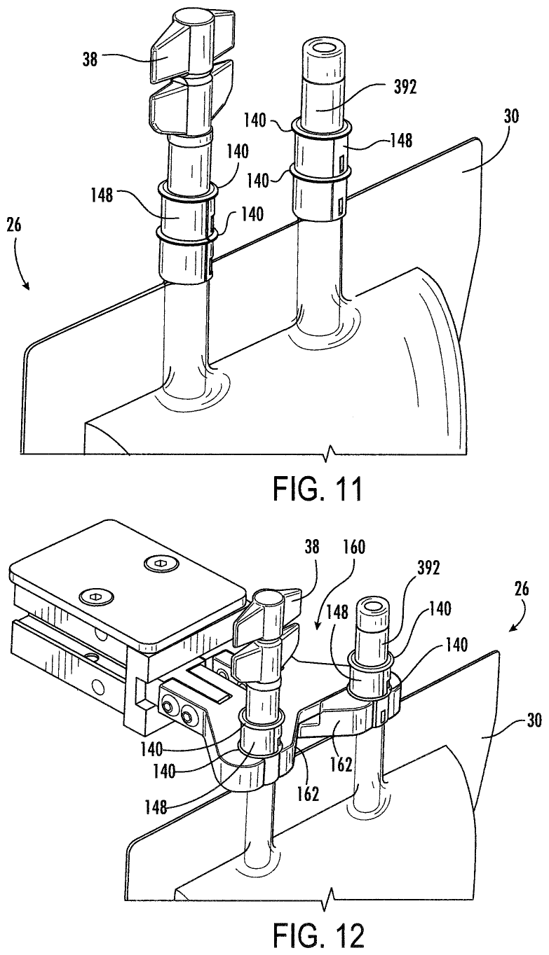

Referring now also to FIG. 11, a ribbed sleeve 148 may be formed around each port 392 of a bag 26. The ribbed sleeve 148 may be held in place via an adhesive, solvent bonding, interference fit, etc. In the example, the projection bearing bodies 142 include ridges 152 on their interior surfaces 154 which may press or “bite” into the port 392 helping to prevent displacement of a sleeve 148 along the port 392. In some embodiments, the ribbed sleeve 148 may be loose on the port 392 and extend from the bag 26 to the stopper 38 (or septum 392). The bag 26 and stopper 38 (or septum 392) may block axial displacement of the ribbed sleeve 148. The ribs 140 may be positioned at even height across the ports 392 of the bag 26.

Referring now also to FIG. 12, in addition to providing a port 392 with ribs 140, a ribbed sleeve 148 may also help constrain the port 392 in a more tightly controlled range of positions. The ports 392 may typically be formed of a relatively flexible and floppy material. The ribbed sleeve 148 may be constructed of a rigid material (e.g. rigid polymer). Thus, the ribbed sleeve 148 may hold the port 392 such that the port 392 tends to extend in a direction parallel to the axial dimension of the sleeve 148. This may assist in ensuring reliable positioning of the port 392 when grasped by a grasper 160.

As best shown in FIG. 12, the projections on the ports 392 may also facilitate handoff between different graspers 160 or other bag retainers. As shown, the jaws 162 of the grasper 160 in the example are closed around port 392 below the first rib 140. The first rib 140 may abut against the jaws 162 inhibiting displacement of the bag 26 along the axial dimension of port 392. Thus, a form closure type arrangement engendered by the port 392 and grasper 160 geometries may be established. The space intermediate the two ribs 140 is unoccupied by a grasper 160. A second grasper 160 may close around the port 392 in the space between the two ribs 140. The first grasper 160 could then release the bag 26. The bag 26 would be inhibited from displacing due to the other rib 140 overhanging the jaws 162 of the second grasper 160. Such ribs 140 may also facilitate use of passive retainers. The ribs 140 may rest upon cradle shaped passive retainers and prevent the bag 26 from falling through the retainer. Additionally, jaws 162 of a grasper 160 may be allowed to loosely hold the ports 392. This may remove the need for a dedicated jaw 162 actuator capable of maintaining a fictional, force closure, type engagement with the port 392. The surfaces of the ribs 140 are slanted or angled to help guide a port 392 into a grasper 160 or other retainer.

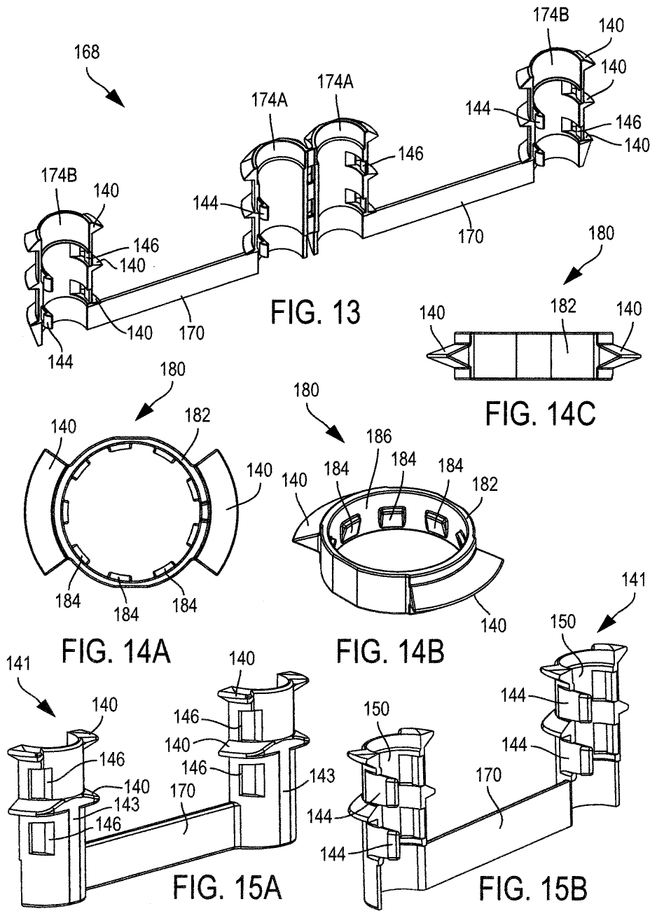

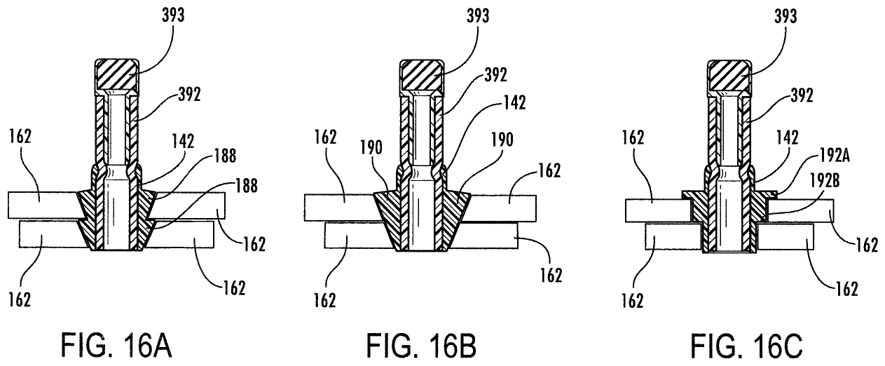

Referring now to FIG. 13, an exemplary ribbing clip 168 is depicted. A ribbing clip 168 may include integrally formed ribbed bodies 174A, B similar to the projection bearing bodies 142 depicted in FIGS. 10A-12. Projection varieties other than ribs 140 may be included in alternative examples (see, e.g., FIGS. 16A-C). The ribbed bodies 174A, B may be included in sets on each half of the ribbing clip 168. The number of ribbed bodies 174A, B in each set may be equal to the number of ports 392 on the bag 26 the ribbing clip 168 is to be coupled to. The ribbing clip 168 may include bridges 170 spacing the ribbed bodies 174A, B of each set apart. Two ribbed bodies 174A at the central region of the ribbing clip 168 may be hinged together. The ribbing clip 168 may be substantially symmetric about a plane of symmetry extending through the axis of the hinge. A living hinge 172 is used in the ribbing clip 168 shown in FIG. 13. Each of the ribbed bodies 172A, B includes a number of ribs 140. Three ribs 140 on each ribbed body 174A, B are included with one rib 140 adjacent the bridge 170, one at the terminal region of the ribbed body 174A, B opposite the bridge 170, and one in a central region of the ribbed body 174A, B. Ports 392 or projection bearing bodies 142 may include any suitable number of projections and other embodiments of projection bearing bodies described herein may include a differing number of projections in alternative embodiments.

Still referring to FIG. 13, the ribbed bodies 172B each include at least one clip projection 144 and at least one retention catch 146. One of the ribbed bodies 172A includes a clip projection 144, the other includes a retention catch 146. As one side of the ribbing clip 168 is folded over against the other, the clip projections 144 may snap into retention catches 146 on the opposing side of the ribbing clip 168. This may allow the ribbing clip 168 to be closed and retained in place about the ports 392 of a bag 26. The ribbed bodies 174A, B may form sleeves similar to the ribbed sleeve 148 of FIG. 10B around ports 392. In other embodiments, ultrasonic welding may be used to couple the sides of the clip 168 together after they have been folded against one another. Solvent bonding, adhesive, or any other suitable coupling method may be used. Projection bearing bodies 142 described herein may be similarly coupled to form a sleeve around a port 392 with a variety of coupling methods such as any of those described above.

Still referring to FIG. 13, the bridges 170 may be relatively resilient and assist in keeping the ports 392 spaced apart at a controlled distance. In some embodiments, the ribbing may be absent from the ribbed bodies 172A, B. The clip 168 would then be a bridging clip which assists in controlling the spacing of the ports 392.

In still other examples, and referring now to FIGS. 14A-C, ribbed rings 180 may be included. The ribbed rings 180 may include a band 182 which includes a set of ribs 140 extending outwardly therefrom. The interior of the band 182 may include a set of teeth 184. The teeth 184 are ramped so as to increase in height from the interior surface 186 of the band 182 as proximity to an end of the band 182 increases. Thus, the band 182 may define a dogged aperture through which a port 392 may extend. A ribbed ring 180 may be slid over a port 392 and the dogged aperture will cause the ribbed ring 180 to frictionally and compressively resist movement relative to the port 392. In some embodiments adhesive may additionally be used or be used in place of the teeth 184. Any desired number of ribbed rings 180 may be installed on a port 392. A fixture may be utilized to assist in creating appropriate positioning and spacing of ribbed rings 180 on a port 392.

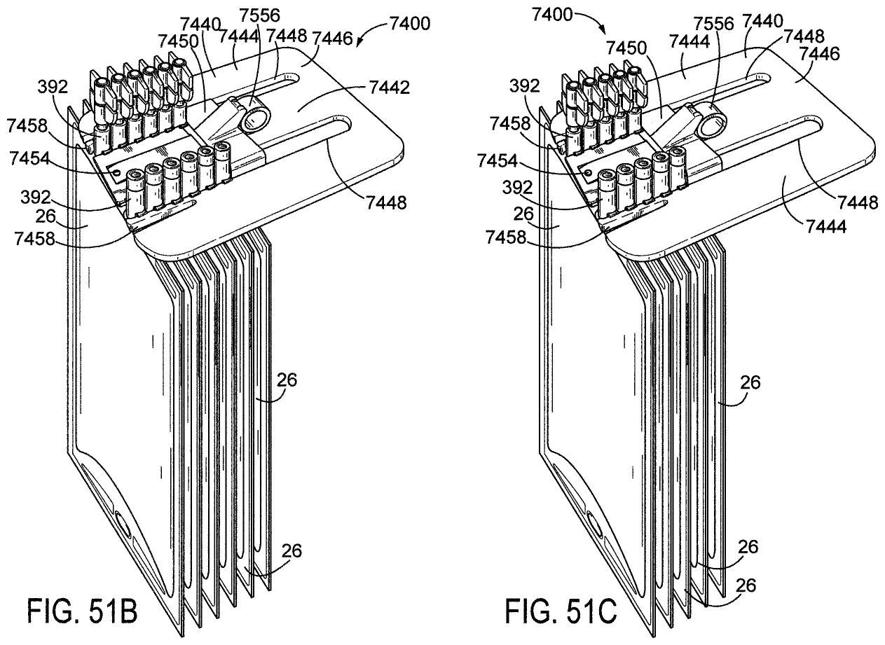

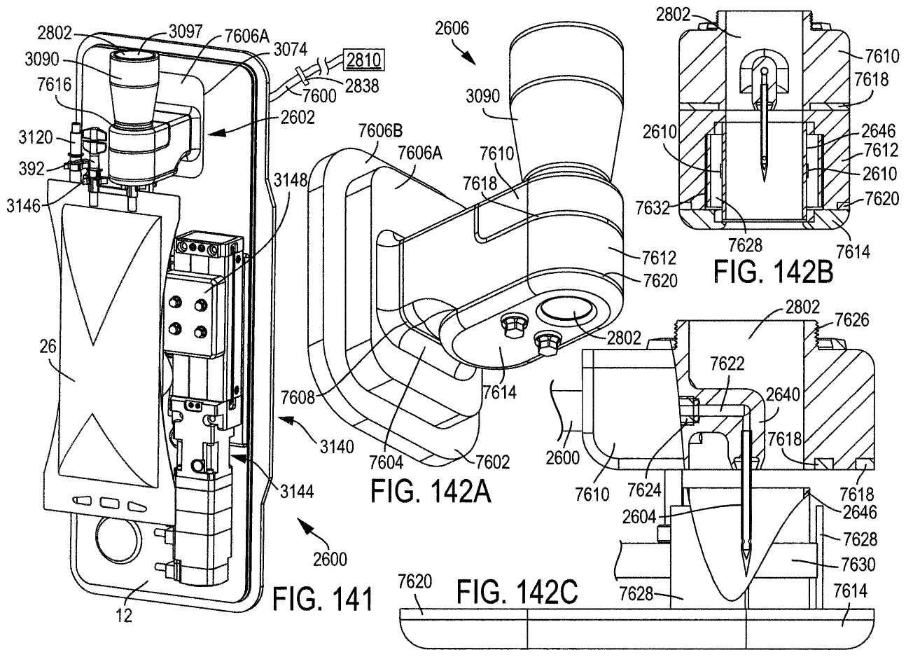

In certain other examples, and referring now to FIGS. 15A-B, ribbed spacers 141 may be included. Ribbed spacers 141 may include ribbed bodies 172 connected by a bridge 170. The ribbed bodies 172 may each include a set of clip projections 144 and a set of retention catches 146. The ribbed bodies 172 may also include arced walls 150. Two ribbed spacers 141 may be coupled to one another. The clip projections 144 and retention catches 146 may be disposed on the ribbed spacer 141 such that two identical ribbed spacers 141 may be clipped together to form ribbed sleeves 148 around ports 392 of a bag 26 (see, e.g., FIG. 141). When coupled together, the pair of ribbed spacers 141 may be referred to as a spacer clip. The clip projections 144 of one ribbed spacer 141 may engage with the retention catches 146 of the companion ribbed spacer 141 to couple the ribbed spacers 141 together on the ports 392 of a bag 26 (other coupling methods such as those mentioned above may also be used). The bridges 170 and sleeves 148 may assist in constraining the ports 392 into a tightly controlled range of positions and into a more tightly controlled range of position relative to one another. This may assist loading of bag 26 into a bag vending assembly 7400 and may facilitate grasping with automation hardware. The ribbed bodies 172 may include flat faces 143 on the exterior of the ribbed bodies 172. The flat faces 143 may run parallel to the bridge 170 and may be on a portion of the ribbed bodies 172 most distal to a plane along which the bridge 170 extends. The flat faces 143 may assist in positioning bags 26 adjacent one another in, for example a bag vending assembly 7400 (see, e.g., FIG. 51C) and may help increase the packing density of the bags 26.

Though described projection bearing bodies 142 shown in FIGS. 10A-B, 16A-18C, the ribbing clip 168 of FIG. 13, the ribbed rings 180 of FIGS. 14A-C, or the ribbed spacers 141 of FIGS. 15A-B are shown as individual components, these components may be provided on the ports 392 of a bag 26 in an overmolding operation in certain examples. Any of the above may be modified to include ridges 152 (see, e.g., FIG. 10A) teeth 184 (see, e.g., FIG. 14B) which bite into the exterior of a port 392 to assist in fixing the component in place on the port 392. Additionally, the height of any of the projection bearing bodies 142 shown in FIGS. 10A-B, 16A-18C, or the height of any ribbing clips 168 of FIG. 13 and ribbed spacers 141 of FIGS. 15A-B may be adjusted. The height may be adjusted such that the entire port 392 is substantially clad or sheathed in the component. One end may be adjacent the bag 26 will another may be adjacent a step provided by a stopper 38 or septum 393 in the port 392. This may similar assist in preventing displacement of the components along the axial dimension of the ports 392.

Referring now to FIGS. 16A-C, as mentioned above, the projections on a port 392 need not exclusively be ribs 140. In some examples, the projections may be barbs 188 as shown in FIG. 16A. The barbs 188 may be ramped so as to decrease in thickness as proximity to the bag 26 increases. The jaws 162 of a gripper 160 (see, e.g., FIG. 12) may be complimentarily angled. When the jaws 162 are closed about the port 392 the ramped surface of the barb 188 may interact with the slanted surface of the jaw 162 to help guide the jaw 162 into place on the port 392. Additionally, the ramped surface of the barb 188 may prevent the bag 26 from displacing about the axis of the port 392 and falling through the grasper 160. The thicker regions of the barb 188 may give the port 392 a width that is greater than the shortest distance between the jaws 162 when the jaws 162 are closed on the port 392. Thus, the jaw 162 may present an interference to such displacement of the bag 26. Alternatively, and as shown in FIG. 16B, the projections on the ports 392 may be wedges 190 which decrease in thickness as proximity to the bag 26 increases. Thus, wedges 190 may similarly inhibit a bag 26 from falling through a gripper 160 when the jaws 162 of that gripper 160 are closed upon the port 392.

In still other examples, the projections may be a set of steps or tiers 192A, B on the port 392. The tiers 192A, B may incrementally increase the width of the port 392 in a stepwise manner. The width of the port 392 may increase as distance from the bag 26 increases. Jaw 162 of a gripper 160 (see, e.g., FIG. 12) may close about the port 392 below a respective tier 192A, B. The step presented by the tier 192A, B may rest upon the jaws 162 of the gripper 160 preventing the bag 26 from falling through the jaws 162. Barbs 188, wedges 190, or tiers 192A, B may be defined on a projection bearing body 142. A ribbing clip 168 may include barbs 188, wedges 190, or tiers 192A, B in place of ribs 140 is some examples.

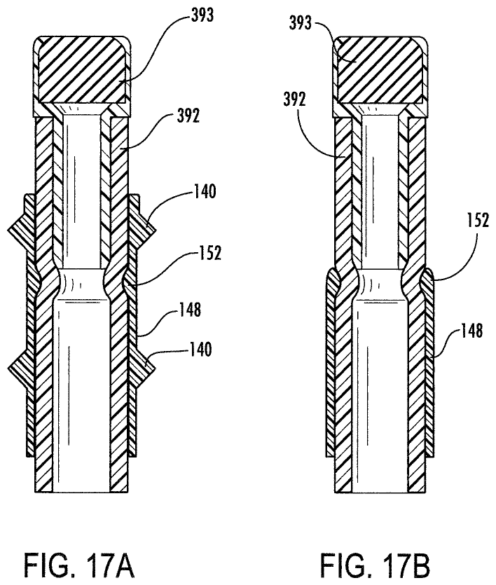

Referring now to FIGS. 17A-B, where projection bearing bodies 142 are used, the interior surfaces of the projection bearing bodies 142 may include interior ridges 152. At least one of the ridges 152 may be positioned to compress the port 392 directly adjacent a rigid insert in the port 392. A septum 393 is depicted in FIGS. 17A-B, but the rigid insert could alternatively be a spike port or stopper 38 (see, e.g., FIG. 4B). The rigid insert may prevent compression of the port 392 by the interior ridge 152. Thus, the projection bearing body 142 may be inhibited from sliding over the portion of the port 392 through which the rigid insert extends. This may assist in fixing the location of the sleeve 148 formed by the projection bearing bodies 142. Such interior ridges 152 may also be included on a ribbing clip 168.

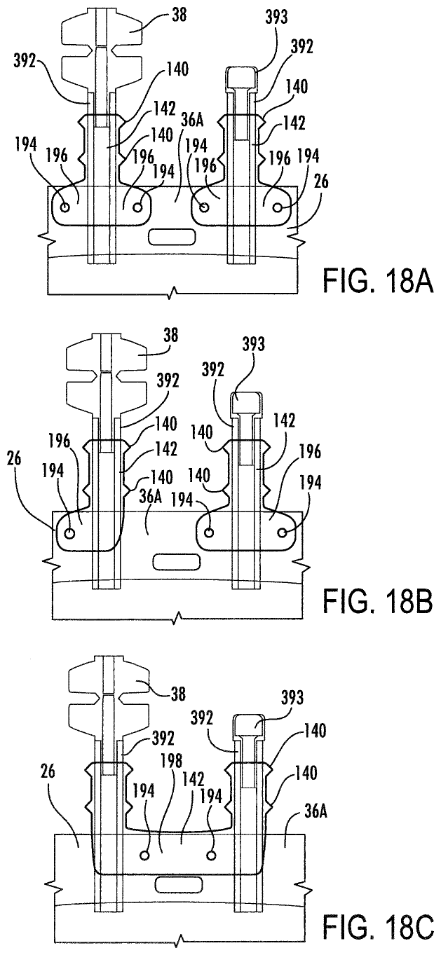

Referring now to FIGS. 18A-C, certain projection bearing bodies 142 are provided in pairs with complimentary snap fit features. One projection bearing body 142 of the pair may include at least one male snap fit protrusion 194 while the other may include a matching number of female snap fit retention catches. The bag 26 may include an enlarged region 36A of the peripheral seal 30 through which the ports 392 extend. The enlarged region 36A have passages therethrough. The male snap fit protrusions 194 of a projection bearing body 142 may extend through the passages in the enlarged region 36A. The snap fit retention catches of a cooperating projection bearing body 142 may be pressed into engagement with the snap fit protrusions 194 extending through the passages. This may form a sleeve around the port 392. It may also fix the position of the sleeve relative to the port 392 as the bag 26 material though which the snap fit engagement is achieved anchors the sleeve in place. The projection bearing bodies 142 may additionally include clip projections 144 and retention catches as described in relation to FIG. 10A-B.

As shown in FIG. 18A, the projection bearing bodies 142 may each include a pair of opposing wings 196. The male and female snap fit features may be defined in the wings 196. Alternatively, and as depicted in FIG. 18B, only one wing 196 may be included on a projection bearing body 142. Projection bearing bodies 142 of differing varieties may be used for each port 392 of the bag 26. In other embodiments, the projection bearing bodies 142 may be included on a connecting bridge 198. The snap fit features may be included in a portion of the connecting bridge 198.

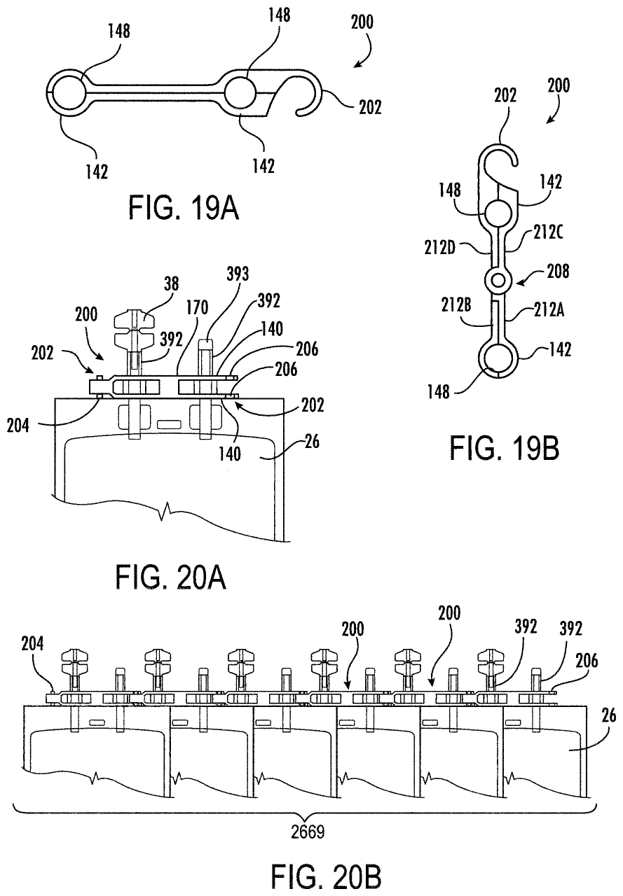

Referring now to FIGS. 19A-20B, in certain examples, projection bearing bodies 142 may be incorporated into a chaining linkage 200. Bags 26 may be coupled together with the chaining linkages 200 to form a linked assembly of bags 2669 (see, e.g., FIG. 20B) or may be placed on bags 26 abreastly coupled to one another in a chain 2670. Chains 2670 of bags 26 are further described in relation to FIGS. 25A-B, for example. Each chaining linkage 200 may include a bridge 170 connecting a set of projection bearing bodies 142. Any desired projection variety may be included on the projection bearing bodies 142. The chaining linkages 200 may also include at least one linkage body 202. In the examples shown in FIGS. 19A-B, the linkage body 202 is a hook. The hook is included as an extension off of one of the projection bearing bodies 142. The hook may receive (snap around) a projection bearing body 142 of a chaining linkage 200 on an adjacent bag 26. This may pivotally couple the adjacent chaining linkages 200. In other embodiments, and as depicted in FIG. 20A, the chaining linkage 200 may include a linkage body 202 extending from the projection bearing bodies 142 on each end of the bridge 170. One of the linkage bodies 202 may include a post 204. The opposing linkage body 202 may include a clip 206 which may engage with a post 204 of an adjacent linkage body 202. Again, a pivotal coupling may be formed between chaining linkages 200 on adjacent bags 26. A linked assembly of bags 2669 coupled together by chaining linkages 200 is depicted in FIG. 20B.

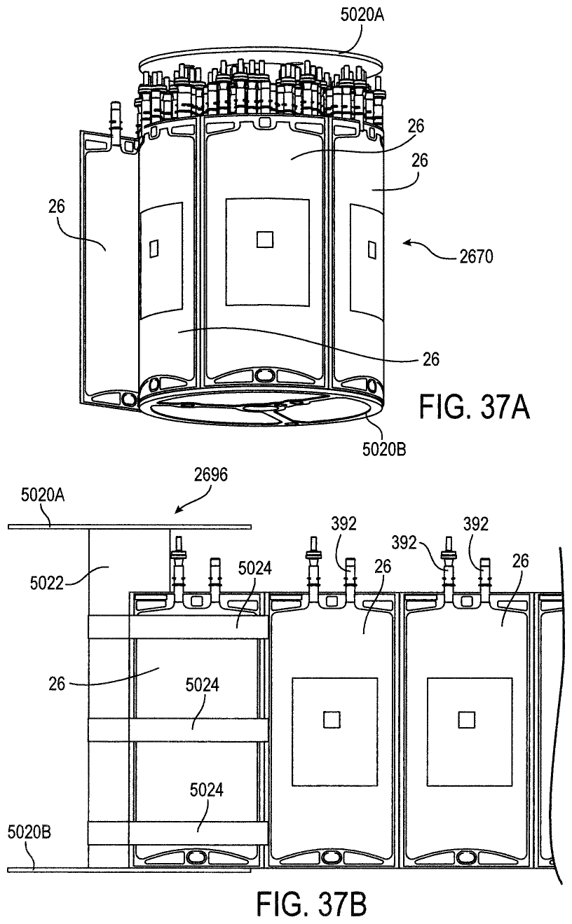

The pivotal coupling established when adjacent chaining linkages 200 are engaged with one another may allow for the bags 26 to be rolled on a spool (see, e.g., FIG. 37A). In some embodiments, at least one hinge 208 may be included in an intermediate portion of the bridge 170 between the projection bearing bodies 142. The hinge(s) 208 may facilitate rolling of a bag 26 onto a spool. The hinge(s) 208 allow the ports 392 of the bag 26 freedom to displace relative to one another. Thus, the ports 392 will more easily assume positions which allow the bag 26 to be curved around the spool. In alternative embodiments, the bridge 170 may be formed of a highly flexible material and the bridge 170 itself may bend to adapt to curvature of the bag 26 on the spool.

Referring primarily to FIGS. 19A-B, the chaining linkages 200 may be an assembly of multiple sections. In FIG. 19A, the chaining linkage 200 includes a first and second portion 210A, B. Alternatively, each portion 210A, B may be integrally formed and connected via a living hinge. The first and second portion 210A, B may be coupled together (e.g. sonically welded, snap fit, solvent bonded, adhered, etc.) around the ports 392 of the bag 26 to form the chaining linkage 200. The ports 392 would extend through sleeves 148 formed by the chaining linkage 200 when the two portions 210A, B are coupled together. The chaining linkage 200 of FIGS. 20A-B may be similarly constructed.

The embodiment of FIG. 19B may include a first side and a second side which are coupled to one another at the hinge 208. The first side may be constructed of a first body 212A and second body 212B. The first and second body 212A, B may be coupled to one another in any suitable manner. The second side may be constructed of a third body 212C and a fourth body 212D which may be coupled together in any suitable manner. The hinge portion of the first side may be pivotally coupled to the hinge portion of the second side to complete the chaining linkage 200.

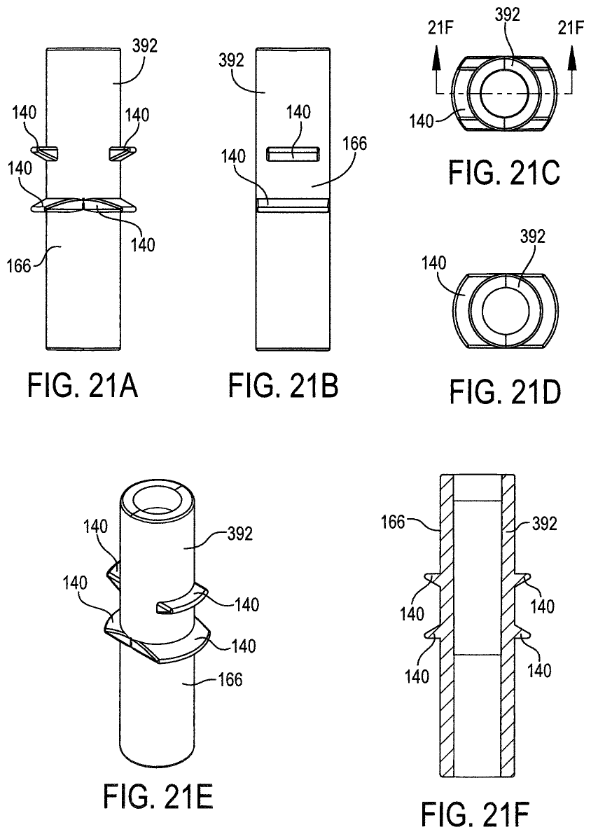

Referring now to FIGS. 21A-F, in certain examples, the ribs 140 may be formed integral to the ports 392. Though the example embodiment is described as including ribs 140 any desired variety of projection may be included and ribs 140 are merely exemplary. The ports 392 may be formed from a rigid material. Various injection moldable plastics may be used such as a polypropylene blend. In certain examples, PolyCine App-147 may be used. The polymer used may be selected based on its ability to bond with the sheeting 44A, B material used in a given bag 26 during welding. As the ports 392 are formed of a rigid material, the ports 392 have less tendency to flop, bow, or bend and have better constrained positions. Ports 392 may optionally be connected via a bridge 170 in some examples.

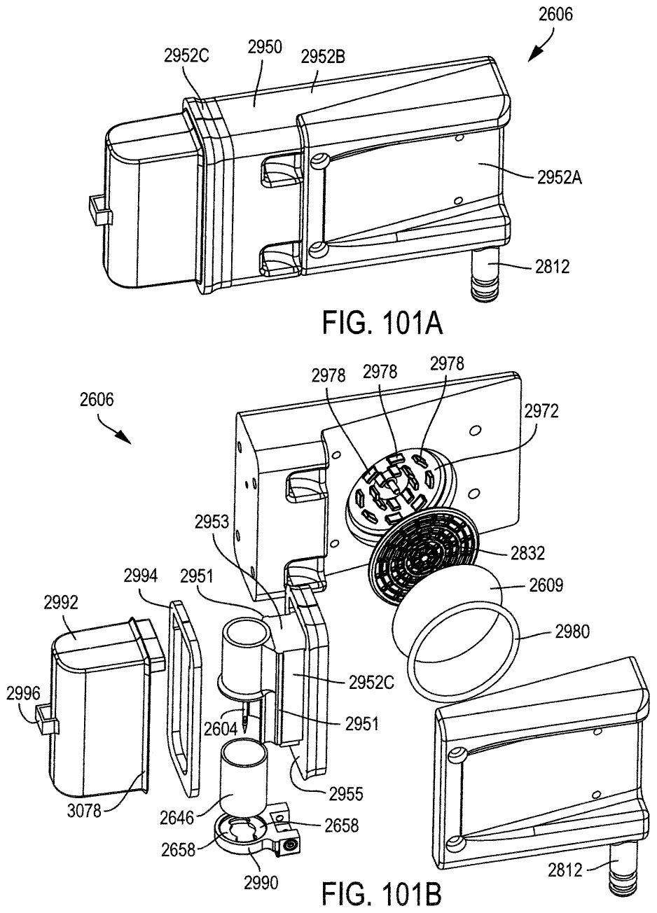

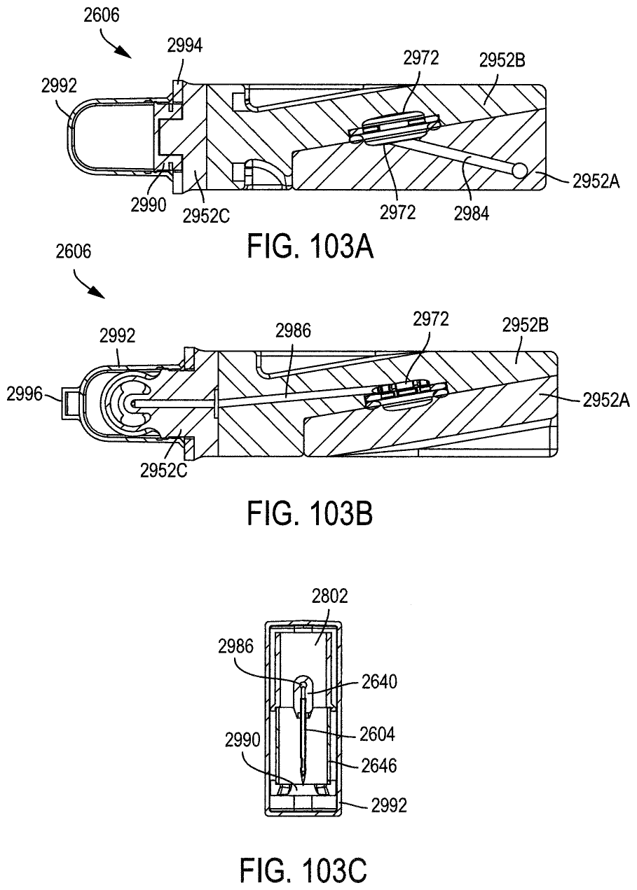

As shown, the ribs 140 may not surround the entirety of the exterior surface 166 of the ports 392 (though could in alternative examples). The example ribs 140 are positioned in pairs. Each pair includes a set of ribs 140 disposed in opposition to one another at the same height on the port 392. One pair of ribs 140 may be present along respective arcs on opposing portions of the exterior surface 166 of the port 392. The other pair of ribs 140 may be present along respective larger arcs on opposing portions of the exterior surface 166 of the port 392. At least the ribs 140 (or other projection) most proximate the septum 393 may be shaped (e.g. extend over a smaller arc) so as to facilitate introduction of the port 392 into an airflow channel 2802 of a cartridge 2606 (see, e.g., FIG. 101B and FIG. 103C).

The port 392 may be symmetric about a medial plane extending along the axial dimension of the port 392. The faces of each set of ribs 140 most proximal to those in the other set of ribs 140 may be slanted away from one another. Thus, the thickness of the ribs 140 may decrease as distance from the exterior surface 166 increases. The slanted faces may assist in guiding jaws 162 of a grasper (or a passive port retaining clip or cradle) into position between the two ribs 140. The faces of each set of ribs 140 most distal to those in the other set ribs 140 may be oriented substantially perpendicular to the axis of the port 392. This may provide a lip which resists slippage of a port 392 through a grasper or passive clip positioned under the rib 140.

The ports 392 may be molded to have a relatively smooth surface finish. A semi-gloss, near glossy, or high gloss finish may be used. Molds for the ports 392 may be polished to an SPI B-1 finish or greater. This may provide a mold with a Ra surface roughness of 0.1 μm or less. In some examples, the mold may be finished to an SPI A-2 surface finish. The smooth finish may facilitate creation of a robust seal when septa 393 (see, e.g., FIG. 7B) or other stoppers 38 (see, e.g., FIG. 7B) are assembled into the ports 392. It may also facilitate use of a wider range of materials for use in molding of the ports 392 while still achieving a robust seal against an insert. Additionally, the flexural modulus of the polymer used for the ports 392 may be in the range of 470-630 MPa (at 23°, 50% rH) though values above and below that range are also possible.

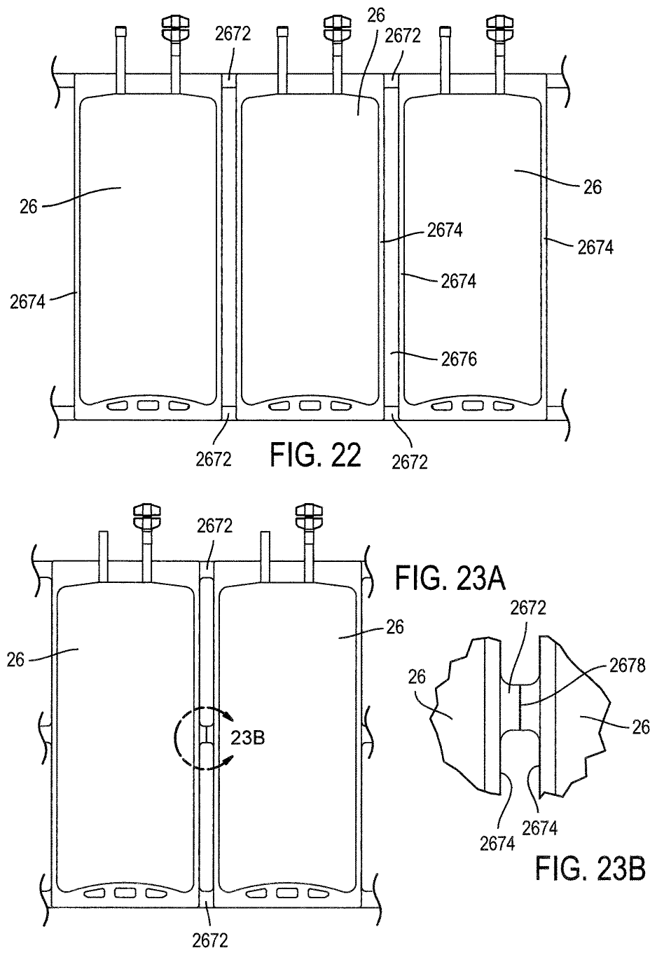

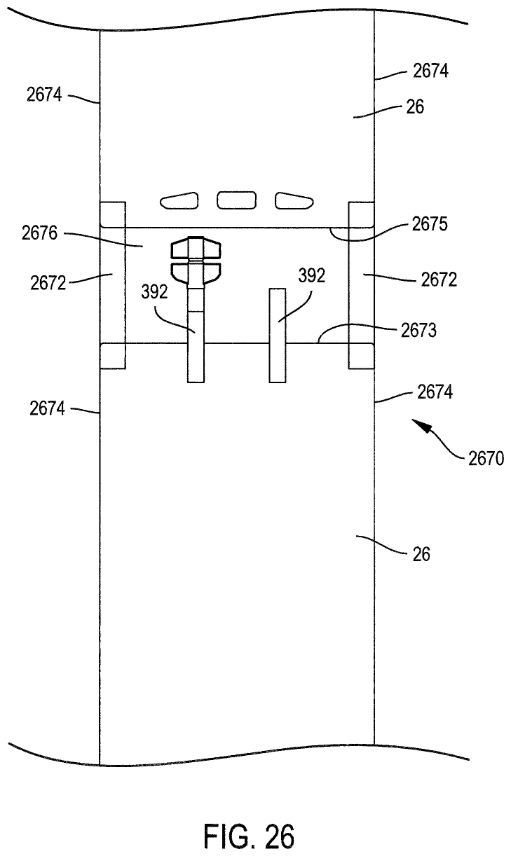

Referring now to FIGS. 22-23B, in certain examples, a system 10 may receive a supply of bags 26 which are provided coupled to one another. The bags 26 may be fed into or installed in the system 10 in a coupled state and may be separated or individualized as needed when filled or prior to being filled. Individualization of each bag 26 may be done in an automated manner (see, e.g., FIG. 74). Bags 26 may, for instance, be provided coupled together in a chain 2670. Portions of example chains 2670 of bags 26 are depicted in FIGS. 22-26. Each bag 26 may be coupled to at least one adjacent bag 26 in a side by side or end to end manner. Where bags 26 are coupled side by side, a chain 2670 may include a number of coupled bags 26 positioned abreast or laterally to one another. Where bags 26 are coupled end to end (see, e.g., FIG. 26), an end of a bag 26 including ports 392 may be coupled to an end of an adjacent bag 26 opposite the ports 392 of that bag 26 to form the chain 2670. The terminal bags 26 on each end of a chain 2670 may only be coupled to a single adjacent bag 26 which in turn is coupled to the rest of the chain 2670. The bag 26 at the beginning of a chain 2670 may optionally be coupled to a leader. The bag 26 at the opposing end of the chain 2670 may be coupled to a sacrificial tail 2683. Any chain 2670 arrangement described herein may, for example, be used with any of the exemplary bags 26 described herein. Any bags 26 of the types described in U.S. Pat. No. 11,965,766, issued Apr. 23, 2024, entitled Medical Treatment System and Methods Using a Plurality of Fluid Lines (Attorney Docket No. 00101.00343.Z55) or U.S. Pat. No. 11,980,587, issued May 14, 2025, entitled Systems, Methods, and Apparatuses for Producing and Packaging Fluids (Attorney Docket No. 00101.00325.AA697) which are each hereby incorporated by reference herein in their entireties may additionally be coupled together to form the chains 2670 described herein.

A chain 2670 may include any desired number of bags 26 and in certain examples may include anywhere from 5-500 bags 26. Some chains 2670 may include 50 bags or more, others may include 100 bags 26 or more. In some embodiments, 3-5 unconsumable bags 26 may be attached to the chain 2670 to form a sacrificial tail 2683 (further described elsewhere herein). The chain 2670 may include, for example, a desired number of bags 26 (e.g. 100 or 50) plus the bags 26 of the sacrificial tail 2683. At least some bags 26 in the chain 2670 may be multi-compartment bags 26 with at least one of the compartments, for example, being filled with a solid or liquid concentrate. Any bags 26 containing concentrate shown or described herein may, for example, be used (see, e.g., FIGS. 4B-C, 6B, 7A, 9A). Single compartment bags 26 are depicted in FIGS. 22-23A solely for exemplary purposes. The bags 26 may, in some embodiments, be provided in a roll or on a reel and may be spooled out to another component of the system 10.

Where compartmented bags 26 are used, the position of the concentrate 42 containing compartment may differ from bag 26 to bag 26. The concentrate 42 containing compartments may be in a staggered pattern over a series of bags 26. This may facilitate spooling of the bags 26 onto a reel. For instance, a concentrate 42 containing compartment for a first bag 26 may be disposed near the bottom of the bag 26. A concentrate 42 containing compartment of a second bag 26 may be disposed near the top of the bag 26. A concentrate containing compartment for a third bag 26 may be disposed intermediate the position of the concentrate 42 containing compartments in the first and second bags 26. The staggered pattern of concentrate 42 containing compartments may repeat over the length of the chain 2670. This may prevent the spool of bags 26 from having a bulged region where all of the concentrate 42 containing compartments are rolled on top of one another.

As shown in FIG. 22, in some embodiments, there may be a gap 2676 between each bag 26 though in alternative embodiments, no such gap 2676 (see, e.g., FIGS. 24A-B) may be present between adjacent bags 26. Where a gap 2676 is present, the lateral edges 2674 of each bag 26 in a chain 2670 may be coupled to respective adjacent bags 26. Each bag 26 may be coupled to an adjacent bag 26 at at least one point along a given lateral edge 2674. In the example embodiment depicted in FIG. 22, adjacent bags 26 are coupled together by bridges 2672 of material at opposing ends of the lateral edges 2674. In the example embodiment in FIG. 23A, adjacent bags 26 are additionally coupled together in a central region of the lateral edges 2674 as well. The bridges 2672 may be formed of the same material from which the bags 26 are made, though a dissimilar material may be used in alternative embodiments.

In some examples, the connection or at least one of the connections between adjacent bags 26 may include a weakened region 2678. In some embodiments, the weakened region 2678 may be a score line. Alternatively and as shown in FIG. 23B, the weakened region 2678 may be perforated. The weakened region 2678 may be formed by cutting at least partially through, punching at least partially through, and/or otherwise weakening the sheeting 44A, B material forming the bags 26. Bags 26 may preferentially separate from the chain 2670 at the weakened region 2678. The weakened region 2678 may be referred to herein as a tear guide in relation to certain exemplary embodiments.

Referring now to FIGS. 24A-25B, in some examples, a bridge 2672 between adjacent bags 26 may be present along the majority or entirety of the lateral edges of adjacent bags 26. As shown in FIG. 24A, bags 26 of the chain 2670 may be coupled together at coupling regions 2677 (e.g. formed integrally with one another or joined by a strip of material) present between each adjacent bag 26. Each coupling region 2677 may include a weakened region 2678 at which two adjacent bags 26 may be more easily separated. A perforation is included in the exemplary embodiment shown in FIGS. 24A-B, however, any suitable weakened region 2678 may be included. Score lines are depicted in FIG. 25B for example. When bags 26 are separated, a portion of the coupling region 2677 may remain with each bag 26. This portion of the coupling region 2677 may also form a section of the peripheral seal 30 of a separated bag 26 and partially define the interior volume of that bag 26. Alternatively, the coupling region 2677 may be an unsealed region 2679 extending between the lateral most peripheral seal 30 edges of adjacent bags 26 of the chain 2670.

The opposing ends (or at least one end) of each coupling region 2677 of exemplary chains 2670 may define guide edges 2698 which lead toward the weakened region 2678. The guide edges 2698 may aid in directing a separating sled (see, e.g., FIGS. 75A-C) toward the weakened region 2678 to facilitate automated separation of bags 26 in the chain 2670. Guide edges 2698 which form a notch or recess substantially in the shape of the Latin character “V” are shown in the example embodiment in FIG. 24A. The guide edges 2698 may also assist in forming a stress concentration where separation preferentially starts as bags 26 are individualized from the chain 2670. Alternatively, the weakened region 2678 may be broken apart or be provided with a pre-separated region 2671 at at least one of the opposing ends of the weakened region 2678. A small fraction of the weakened region 2678 may be pre-separated (e.g. less than 5%). This may similarly facilitate automated separation of bags 26 from the chain 2670. A pre-separated region 2671 in combination with guide edges 2689 may also be used in certain examples.

Referring now to FIG. 26, a portion of another exemplary chain 2670 of bags 26 is depicted. The chain 2670 in FIG. 26 includes bags 26 coupled in an end to end as opposed to a side by side manner. A gap 2676 between each bag 26 may be included to accommodate any ports 392 of the bags 26. The gap 2676 may be sized to be slightly larger than the longest projection distance of a port 392 from the bag 26. Each bag 26 may be coupled to an adjacent bag 26 by at least one bridge 2672 of material. In the example embodiment depicted in FIG. 26, two bridges 2672 are included. Each bag 26 may include a ported end 2673 and an opposing second end 2675. Bridge(s) 2672 may extend from the ported end 2673 to the second end 2675 of an adjacent bag 26 to form the chain 2670. In the example, the bridges 2672 are arranged such that their most lateral edges are in line with the lateral edges 2674 of the bags 26 forming the chain 2670.

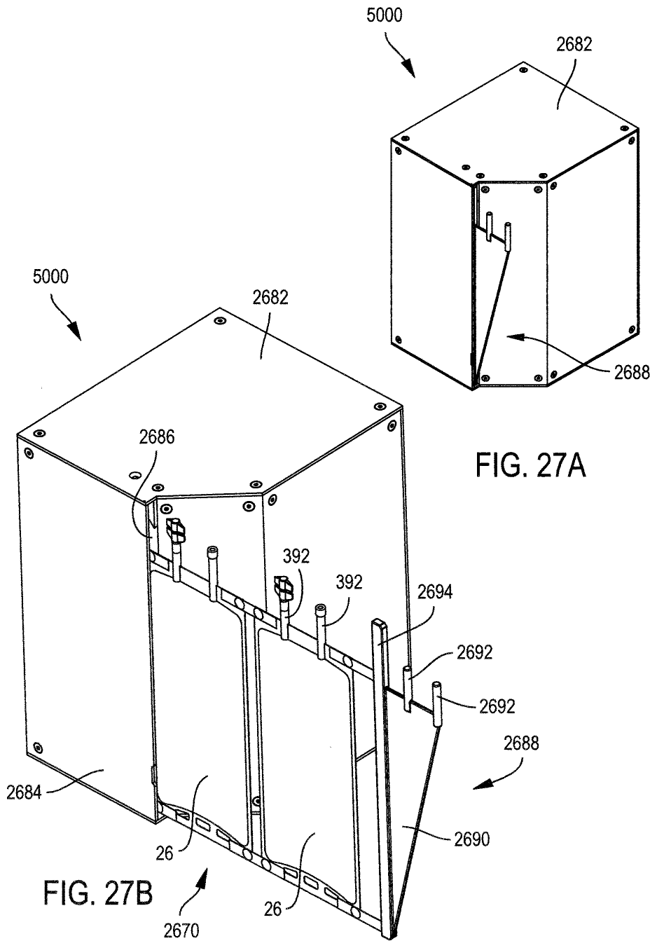

Referring now to FIGS. 27A-29, in some embodiments, chains 2670 of bags 26 may be provided in a dispenser 5000. Example dispensers 5000 may include a housing 2682. The housing 2682 may enclose a chain 2670 of bags 26 which may, for example, be placed on a reel or spool 2696 which may rotate within the housing 2682. The dispenser 5000 may include an outlet 2684. The outlet 2684 may include a slit 2686 through which bags 26 may be dispensed out of the dispenser 5000. The leading end of the chain 2670 of bags 26 may be attached to a leader 2688. The leader 2688 may include a main body 2690 with at least one dummy port 2692 projecting therefrom. The dummy port(s) 2692 may be sized and spaced to mimic the ports 392 on bags 26 of a chain 2670 such that automation equipment of the system 10 may easily interface with the leader 2688. For example, a grasper of a bag retainer assembly 4306 (see, e.g., FIG. 68A) may approach and grasp the dummy port(s) 2692 allowing the leader 2688 to be displaced within the system 10 in the same manner as a bag 26. As the leader 2688 is displaced, the chain 2670 of bags 26 may advance out of the slit 2686 and into the system 10 as shown in, for example, FIG. 27B.

Example leaders 2688 may also include a sealing member 2694. The sealing member 2694 may be a bar or plug which seats in the slit 2686 of the dispenser 5000 until the dispenser 2680 is ready for use. The sealing member 2694 may be formed of or covered at least partially with a complaint material. In certain examples, the walls of the slit 2686 may alternatively or additionally be covered by a compliant material. When in place within the slit 2686 the complaint material on the sealing member 2694 may be compressed against the walls of the slit 2686 and form an environmental seal which separates the interior of the dispenser 5000 from the surrounding environment. The dispenser 5000 may be provided in a terminally sterilized state (e.g. within an over pack) and the interior of the dispenser 5000 may remain in this state until the sealing member 2694 is dislodged from the slit 2686.

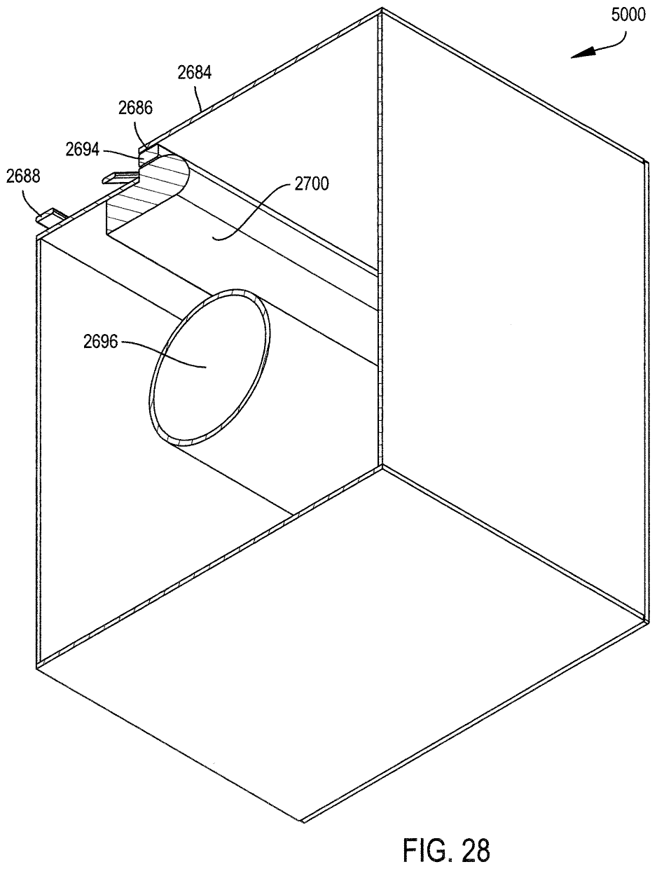

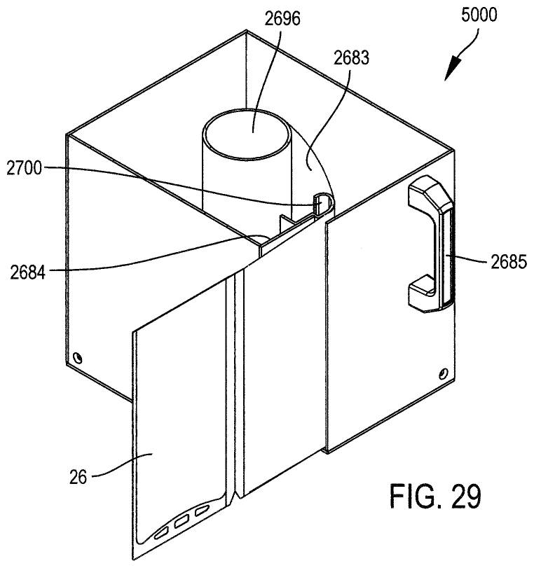

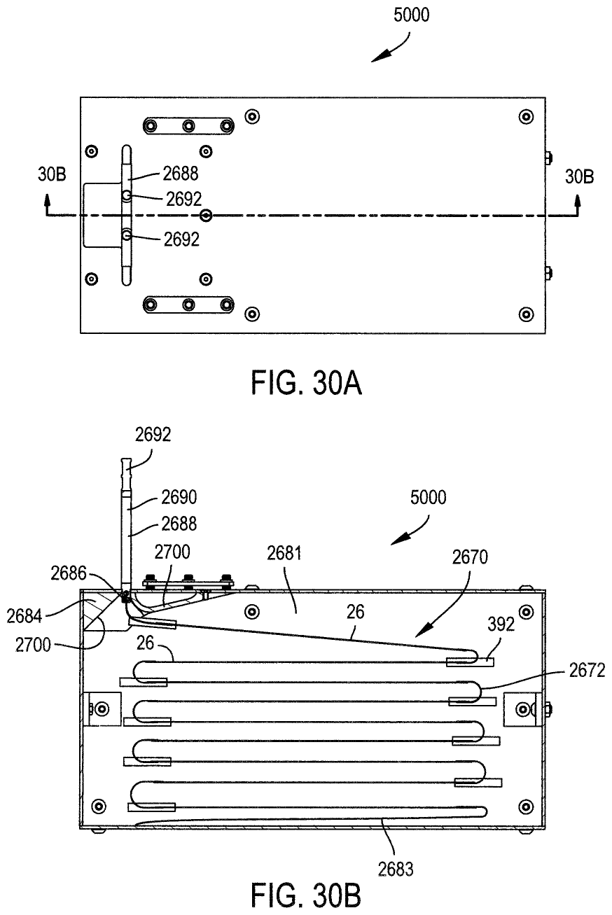

With reference primarily to FIG. 28, a sectioned view of the example dispenser 5000 shown in FIGS. 27A-B is depicted. The chain 2670 of bags 26 is omitted to better illustrate the interior of the dispenser 5000. The leader 2688 is shown in place with the plug portion 2694 of the leader 2688 sealing the slit 2686 of the dispenser 5000. As shown, the dispenser 5000 includes a reel 2696. The chain 2670 of bags 26 may be wrapped around the reel 2696 when packaged into the dispenser 5000 such that it may be spooled out of the dispenser 5000 during use. In some embodiments, the reel 2696 may be paired with a tensioning assembly 5100 (see, e.g., FIGS. 41A-B) so that the bags 26 are kept under some degree of tension as they are spooled out of the dispenser 5000. The outlet 2684 of the dispenser 5000 may include one or more guide 2700 (see also, e.g., FIG. 30B). Referring now also FIG. 29, guides 2700 may be included to mitigate potential for bags 26 to snag or catch as they are advanced out of a dispenser 5000. In various embodiments, the guides 2700 may be formed of (or at least partially covered with) a low friction coefficient material such as PTFE. Walls of the dispenser 5000 may similarly be formed or at least partially covered with such a material.

Referring now to FIGS. 27B-29, the last bag 26 in the chain 2670 may be coupled to a sacrificial tail 2683 (see, e.g., FIG. 29, FIG. 30B). The sacrificial tail 2683 may couple to a portion of a reel 2696 or dispenser 5000. The sacrificial tail 2683 may provide a length of material which may be at least long enough to reach the slit 2686 or dispensing outlet of the dispenser 5000. As the chain 2670 is consumed, the sacrificial tail 2683 allows the final bags 26 of the chain 2670 to be advanced through the outlet 2684 and out of the slit 2686 as best shown in FIG. 29. The sacrificial tail 2683 may remain in the dispenser 5000 once the bags 26 in the chain 2670 have been completely consumed. In some embodiments, a tensioner assembly 5100 (see, e.g., FIGS. 41A-B) may be included and would automatically retract the sacrificial tail 2683 into the dispenser 5000 when the supply of bags 26 in the dispenser 5000 has been exhausted.

In some examples, the sacrificial tail 2683 may be a set of bags 26. The bags 26 forming the sacrificial tail 2683 may optionally be simplified. For example, the bags 26 in the sacrificial tail 2683 may not include ports 392 and may include a single interior compartment. Weakened regions between the bags 26 forming the sacrificial tail 2683 may be absent. The bags 26 forming a sacrificial tail 2683 may include reinforced couplings therebetween. Alternatively, the sacrificial tail 2683 may be a strip of sheeting 44A, B material which has no bags 26 defined therein (see, e.g., FIG. 29). In still other embodiments, the sacrificial tail 2683 may include a sacrificial bag 26 adjacent a last consumable bag 26 in the chain 2670. The remainder of the sacrificial tail 2683 may be plain sheeting 44A, B. In other embodiments, the sacrificial tail 2683 may be sheeting 44A, B which includes a set of ports 392 coupled thereto. The ports 392 may be spaced from one another at a spacing which mimics that of ports 392 on a bag 26. The ports 392 may be positioned in the sacrificial tail 2683 at a distance from the last consumable bag 26 which is substantially equal to the distance between ports 392 on adjacent bags 26 in the chain 2670. Including a sacrificial bag 26 or sacrificial ports 392 in the sacrificial tail 2683 may allow the sacrificial tail 2683 to be more easily held in place as the last consumable bag 26 is removed from the chain 2670. The sacrificial tail 2683 may be coupled to the reel 2696 in any suitable manner. In some embodiments, a tape or adhesive may be used.

Referring now to FIGS. 30A-B, another example embodiment of a dispenser 5000 is depicted. The example dispenser 5000 includes an interior bay 2681 sized to accept a chain 2670 of bags 26 coupled together in an end to end manner. The bags 26 may be placed atop one another such that ports 392 of adjacent bags 26 in the chain 2670 face opposite directions. The bridges 2672 of the chain 2670 may be sufficiently flexible to allow the chain 2670 to be folded upon itself at the bridges 2672 such that the bags 26 may be placed in the bay 2681 in a stacked configuration. The bags 26 are shown with some spacing between one another in FIG. 30B for illustrative purposes, but would rest upon one another in practice.

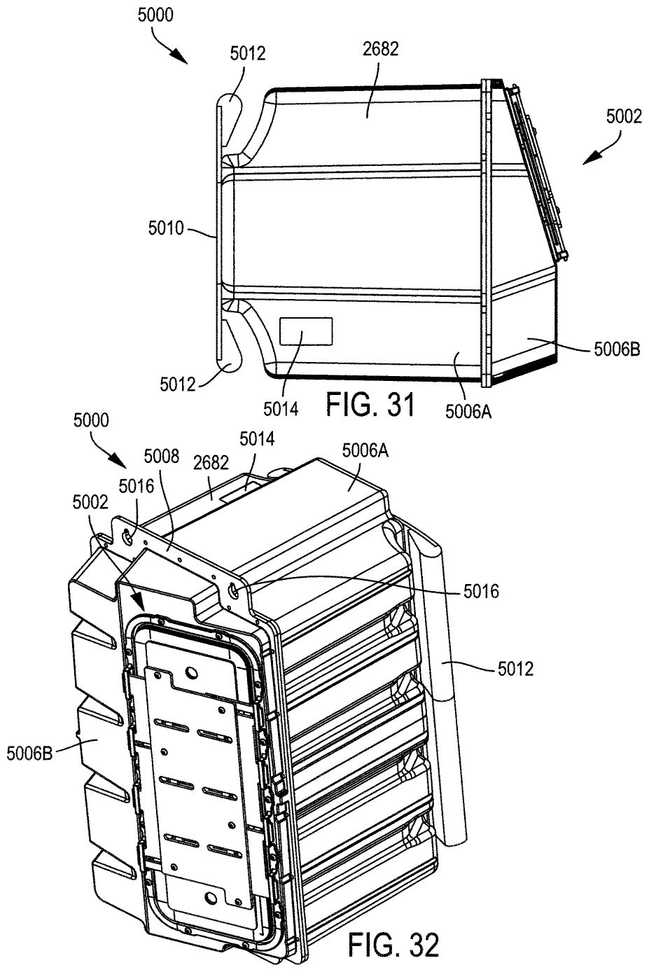

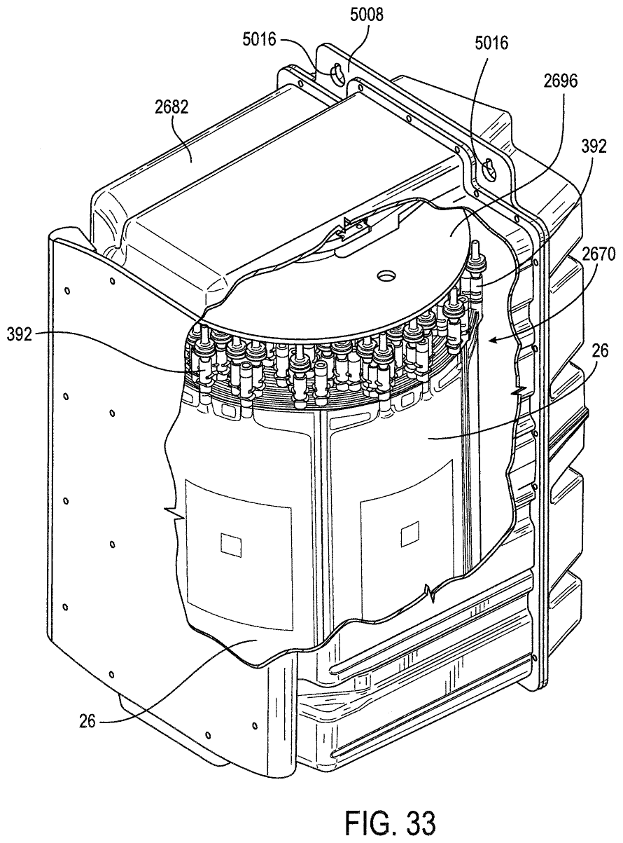

Referring now to FIGS. 31-32, another exemplary dispenser 5000 is depicted. As shown, a dispenser 5000 may include a housing 2682. A chain 2670 of bags 26 coupled to a reel 2696 may be included in the housing 2682 (best shown in FIG. 33). The housing 2682 may include a side panel 5010 including a set of handles 5012. The housing 2682 may be formed of a first housing portion 5006A and a second housing portion 5006B which may each be injection molded components. The handles 5012 may be included on a face of the first housing portion 5006A most distal the second housing portion 5006B. In some examples, one or more handle or grasping recess may be included near the top of the dispenser 5000. The first and second portion 5006A, B may be corrugated or ridged to add strength. In some examples, a recess in a series of corrugations for the first portion 5006A may double as a handle for lifting the dispenser 5000.

The first and second housing portion 5006A, B may be coupled together with fasteners or in any other suitable manner. One or more compliant member may be included to form a fluid tight seal between the first and second housing portions 5006A, B when the housing 2682 is assembled. A flange 5008 may be included in at least one of the first and second housing portions 5006A, B along a portion of the peripheral edge of the respective housing portion 5006A, B. The flange 5008 may include a set of retaining apertures which in the example are depicted as pin retainers 5016. The pin retainers 5016 may be passages through the flange 5008 which have a wider portion and a narrower portion. The pin retainers 5016 may be shaped as teardrop shaped passages through the flange 5008 in some embodiments. Though two pin retainers 5016 are shown, the flange 5008 may include additional pin retainers 5016.

The housing 2682 may also include at least one indicia 5014 such as barcodes, QR codes, GS1 codes, data matrices, bokodes, RFID tag, NFC tag, etc. The indicia 5014 may include various information about the dispenser 5000. For example, the indicia 5014 may include a unique dispenser 5000 identity, a lot identity for the dispenser 5000, production line identity, manufacturing date, an expiration or use by date, information defining the type of bags 26 contained therein (e.g. the fill capacity and type or volume of concentrate in the bags 26), number of bags 26 contained in the dispenser 5000, etc. Where multiple varieties or dispensers 5000 are accepted by a system 10, the indicia may also specify what variety the particular dispenser 5000 is. For example, where systems 10 accept dispensers 5000 of chained bags 26 and individual bags 26, the indicia 5014 may define the if the bags 26 are chained or separated. Where multiple dispensing mechanisms are possible depending on the dispenser 5000 variety, the indicia 5014 may also document the type of mechanism contained in the dispenser 5000 so that automation equipment may be operated by the control system 15 to appropriately interface with the dispenser 5000.

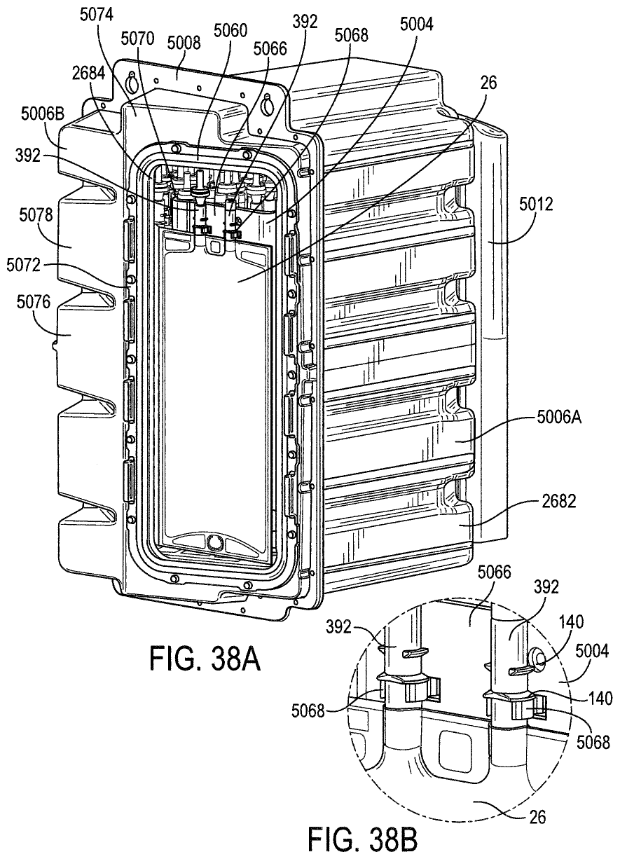

The housing 2682 may also include a removable cover assembly 5002. The removable cover assembly 5002 may be disposed on the side of the dispenser 5000 opposite the handles 5012. The removable cover assembly 5002 may cover and block the outlet 2684 (see, e.g., FIG. 34C) of the dispenser 5000 during shipping and handling of the dispenser 5000. The removable cover assembly 5002 may preferably be machine removable and arranged to make manual removal difficult without a tool. The handles 5012 may be near or on the opposing side to direct user contact away from removable cover assembly 5002. Typically, a lock assembly 5090 may be included which may allow the removable cover assembly 5002 to be locked in place on the dispenser 5000. The removable cover assembly 5002 may include or compress against one or more sealing member 5060 (see, e.g., FIG. 38A) which isolates the interior volume of the dispenser 5000 from the surrounding environment when the removable cover assembly 5002 is in place. Thus, the removable cover assembly 5002 may maintain the interior volume of the dispenser 5000 in a sterilized state.

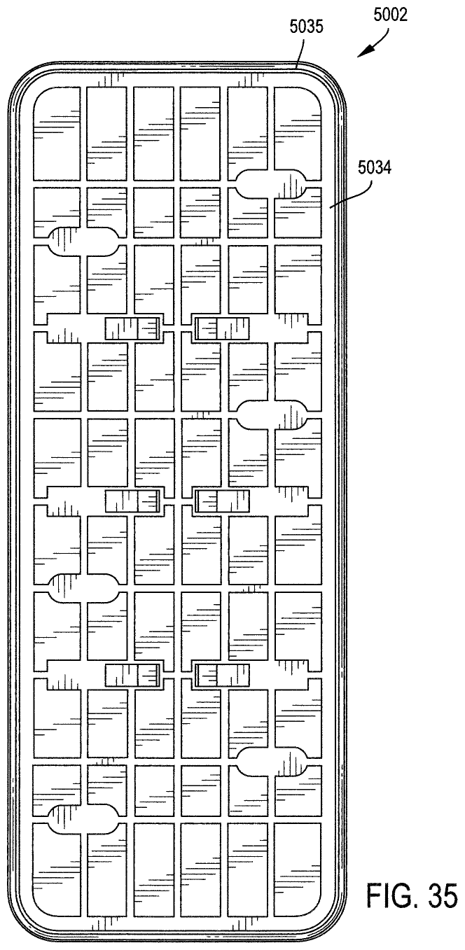

Referring now to FIGS. 34A-35, the removable cover assembly 5002 may include an overlay body 5030, a set of lock bodies 5032A, B, and a backing body 5034. The locking bodies 5032A, B may be disposed intermediate the overlay body 5030 and the backing or sealing body 5034. The backing body 5034 may have a sealing member 5035 or gasket (best shown in FIG. 35) about the periphery of the side of the backing body 5034 opposite the locking bodies 5032A, B. The locking bodies 5032A, B may be captured between the overlay body 5030 and backing body 5034 when the overlay body 5030 is coupled to the backing body 5034. In the example, the overlay body 5030 is coupled to the backing body 5034 via fasteners 5036. In alternative embodiments, the overlay body 5030 may be coupled to the backing body 5034 via welding (e.g. sonic), adhesive, solvent bonding, rivets, heat stake, or any other suitable manner. Each locking body 5032A, B may include a set of bolt projections 5050.

As shown, the example overlay body 5030 is a plate and includes a number of overlay slits 5038. The backing body 5034 includes a number of variable depth troughs 5042 recessed therein. The overlay slits 5038 may be aligned with the troughs 5042 when the removable cover assembly 5002 is assembled. The locking bodies 5032A, B each include a set of apertures 5040. When the removable cover assembly 5002 is in an assembled state, the apertures 5040 may be in alignment with the overlay slits 5038 and troughs 5042.

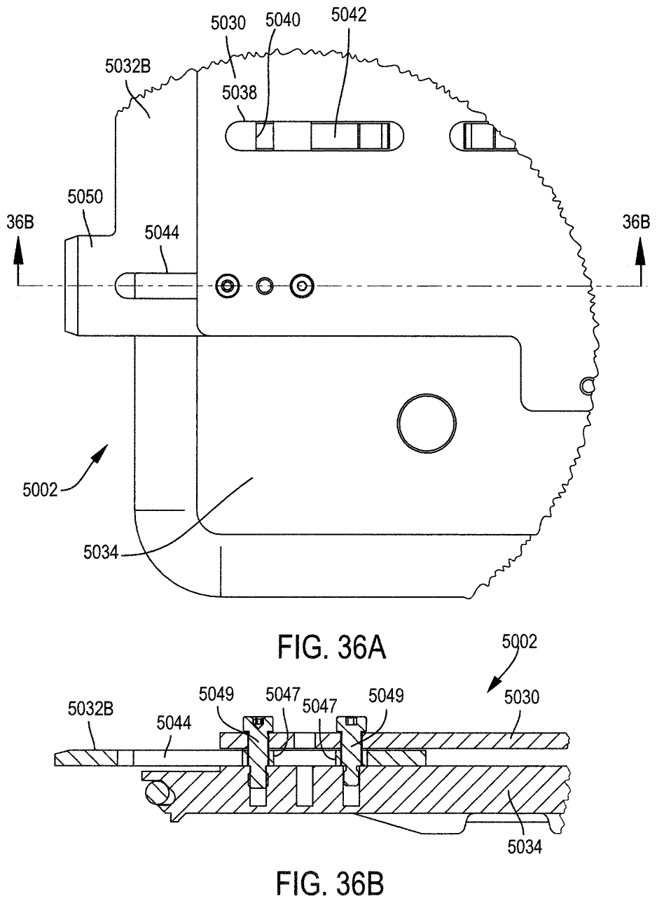

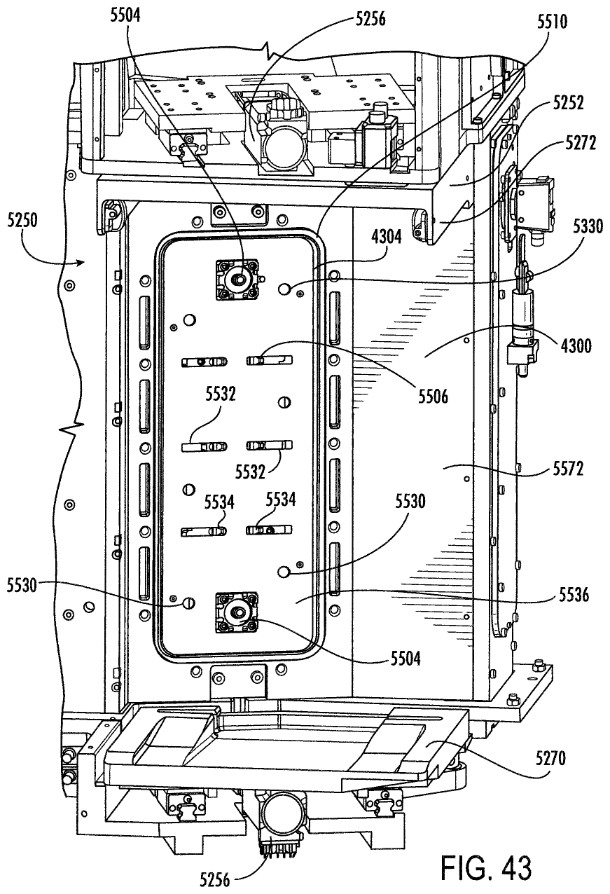

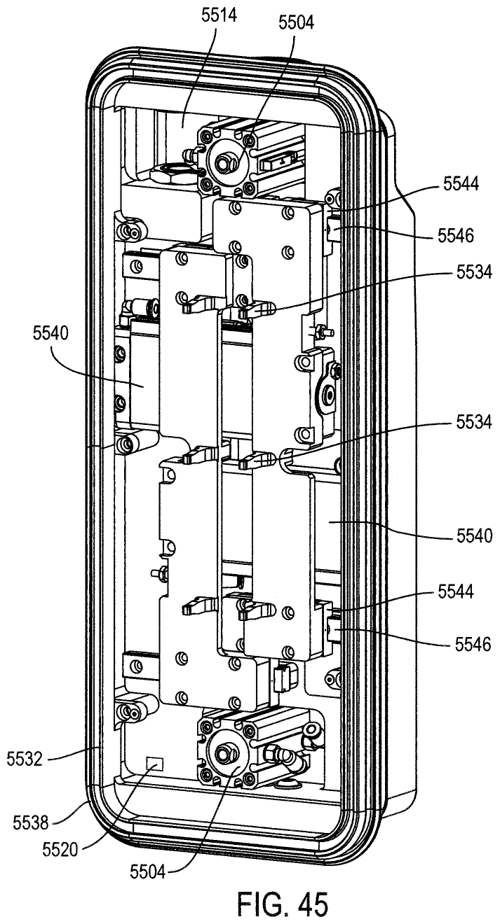

Each locking body 5032A, B also includes a number of guide slots 5044. The guide slots 5044 may accept respective elongate guide rails 5046 which extend proud of the adjacent face of the backing body 5034. Alternatively, a roller arrangement may be used in place of the guide rails 5046 (see, e.g., FIGS. 36A-B). A sanitary interface assembly 5500 (see, e.g., FIG. 2) may include a number of projections 5534 (e.g. pins, rods, fingers see FIG. 43) which are translationally displaceable via actuators 5540 (see, e.g., FIG. 45) in the sanitary interface assembly 5500. A projection 5534 may be passed into each of the overlay slits 5038, through the aligned aperture 5040 in a lock body 5032A, B and to the trough 5042. Translational displacement of the projections 5534 would engender a translational displacement of the lock body 5032A, B through which it extends. Thus, the lock bodies 5032A, B may be translated toward and apart from one another. The lock bodies 5032A, B may be displaced until the ends of the guide slots 5044 collide with edge of the guide rails 5046. Thus, the displacement range of the lock bodies 5032A, B may be limited.

In the spread apart position (see, e.g. FIG. 34A), the bolt projections 5050 of the lock bodies 5032A, B may extend into respective bolt retainers 5052 defined in a rim 5054 surrounding the outlet 2684 of the dispenser 5000. The rim 5054 may be constructed of one more pieces of rigid material and may be rigid plastic or metallic (e.g. stainless steel or anodized aluminum) in some embodiments. As shown, the terminal regions 5056 of the bolt projections 5050 may be ramped. As the bolt projections 5050 are advanced into the bolt retainers 5052, the ramped terminal regions 5056 may cause the removable cover assembly 5002 to be driven or wedged against the housing 2682. This, in turn, may press a sealing member 5035 (best shown in FIG. 35) of the backing body 5034 against a surface of the housing 2682 surrounding the outlet 2684. Thus, when the lock bodies 5032A, B are in a spread apart or locked state, a fluid tight seal may be formed between the removable cover assembly 5002 and the interior of the dispenser 5000.

The lock bodies 5032A, B may be considered to be in an unlocked state or position when retracted toward one another (see, e.g., FIG. 34B). In the unlocked position, the lock bodies 5032A, B may be out of contact with the bolt retainers 5052 of the rim 5054. The removable cover assembly 5002 may be free to be removed from the dispenser 5000 when the lock bodies 5032A, B are disengaged from the bolt retainers 5052.

Referring now to FIGS. 36A-B a portion of an alternative removable cover assembly 5002 and cross-sectional view thereof are respectively depicted. As shown, the guide slots 5044 of the lock bodies 5032A, B do not interface with guide rails 5046 of the backing body 5034 in FIGS. 36A-B. A set of rollers 5047 are included and the guide slot 5044 rides along the rollers 5047 as the lock bodies 5032A, B are displaced. The rollers 5047 may be coupled in place via shoulder bolts 5049 extending through the overlay body 5030. Alternatively, the rollers 5047 may be placed on bosses projecting from the backing body 5034.

Referring now to FIGS. 37A-B, an exemplary chain 2670 of bags 26 on a reel 2696 is depicted. The chain 2670 may include any desired number of bags 26 and the dispenser 5000 may be sized accordingly. Preferably, the weight of the dispenser 5000 with a full chain 2670 of bags 26 may be under 10 kg. Though the bags 26 depicted are those shown in FIGS. 7A-B, any bags 26 shown or described herein may be included and may include any type of concentrate described herein. Bags 26 forming the chain 2670 may have a capacity of 50 ml, 100 ml, 250 ml, 500 ml, 1000 ml, 2000 ml, or 5000 ml, though other sizes may also be used. The example includes 1000 ml bags 26.

As shown, the reel 2696 includes a set of opposing end flanges 5020A, B. A barrel or core 5022 extends between the end flanges 5020A, B. The chain 2670 of bags 26 is shown coupled to the core 5022 of the reel 2696 by adhesive bearing tape 5024 in the example embodiment. Any other cleanroom suitable manner of anchoring the chain 2670 to the core 5022 may be used. Though the portion of the chain 2670 coupled to the core 5022 is a series of bags 26, any sacrificial tail 2683 shown or described herein may be used.

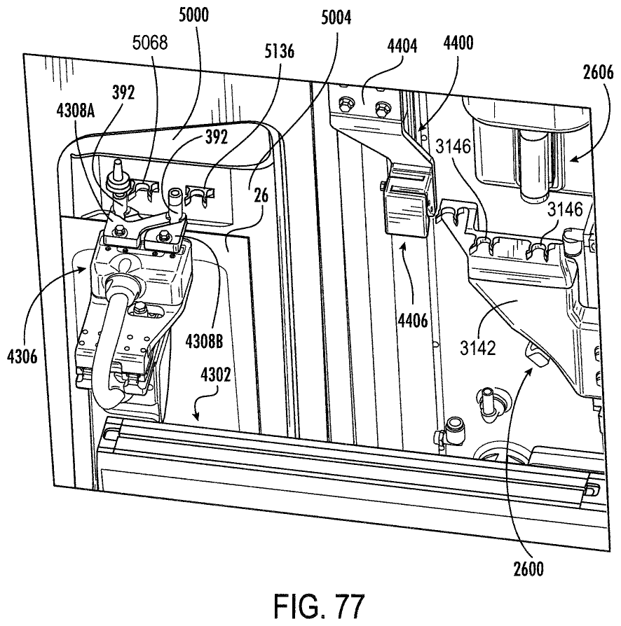

Referring now to FIG. 38A-B, when the removable cover assembly 5002 has been unlocked from the dispenser 5000 and removed, the chain 2670 of bags 26 within the dispenser 5000 may be accessed. As shown, the dispenser 5000 includes a bag presenter 5004. The bag presenter 5004 may be coupled to the housing 2682 or may be formed as an integral part of the first or second housing portion 5006A, B. A bag presenter 5004 may include a guide body 5066 on which a set of port retainers 5068 are disposed. The ports 392 of a lead bag 26 in the chain 2670 may be engaged with the port retainers 5068. This may hold the lead bag 26 in place against any bias force exerted against the chain 2670 by a tensioning assembly 5100 (see, e.g., FIG. 41A-B) of the dispenser 5000. The port retainers 5068 may be the same as those included on the fill displacement stage 3142 and may passively retain the ports 392 of the lead bag 26. The port retainers 5068 may keep the lead bag 26 in an expected position within the dispenser 5000. This may facilitate retrieval of the lead bag 26 by a bag retention assembly 4306 (see, e.g., FIG. 77) of a bag displacement assembly 2618 of the system 10.

The guide body 5066 may be formed as a cantilevered panel. The unsupported end of the guide body 5066 may be rounded or include a lip 5070. The guide body 5066 may also be formed of a low friction coefficient material such as PTFE in some examples. As the chain 2670 is pulled out of the dispenser 5000, the guide body 5066 may assist in directing the chain 2670 as it unravels from the reel 2696. The guide body 5066 may constrain the chain 2670 such that it will tend to exit the dispenser 5000 near the upstream edge 5072 of the outlet 2684. As shown, the outlet 2684 of the dispenser 5000 may be included in an angled face 5074 of the housing 2682. The angled face 5074 may be at a 10-25° angle (e.g. 16.5°) to a second portion 5078 of the front face 5076 of the housing 2682. The second portion of the front face 5076 may be substantially parallel to the opposing side of the housing 2682 including the side panel 5010. The chain 2670 of bags 26 may generally be removed from the dispenser 5000 such that the chain 2670 extends out of the dispenser 5000 substantially parallel (e.g. within 15°) to the second portion 5078 of the front face 5076. Providing the angled face 5074 may assist in providing clearance for the chain 2670 as the chain 2670 is advanced out of the dispenser 5000. The angled face 5074 may further help encourage the chain 2670 to extend through the outlet 2684 near the upstream edge 5072 of the outlet 2684. The angled face 5074 also allows the bag presenter 5004 to be recessed with respect to the exit point of the chain 2670 from the outlet 2684 of the dispenser 5000. This may help to inhibit bags 26 from snagging on the port retainers 5068 as the chain 2670 is consumed.

In certain examples, the port retainers 5068 may be pivotally coupled to the bag presenter 5004 and may be pivoted against the main portion of the bag presenter 5004 after the lead bag 26 is removed. In some embodiments, a bias member (e.g. torsion spring) may be associated with each port retainer 5068 to assist in displacing the port retainers 5068 to a stowed position once the lead bag 26 has been collected.

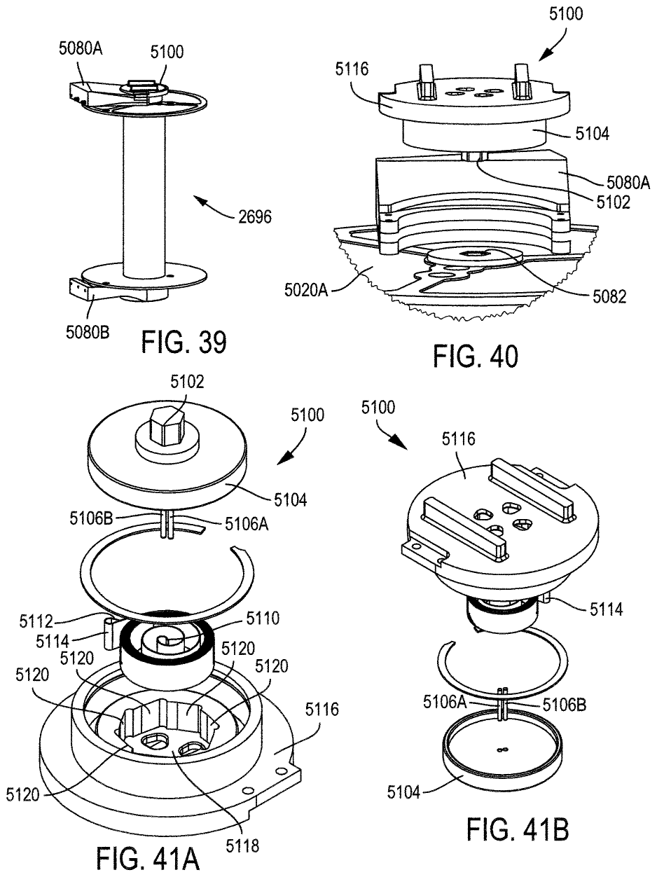

Referring now to FIGS. 39-41B, the spool 2696 of the dispenser 5000 may be associated with a tensioner assembly 5100. As shown, the spool 2969 may be retained within the dispenser 5000 by spool brackets 5080A, B. A plain bearing type interface may be present between the spool flange 5020B and spool bracket 5080B. The spool flange 5020B may, for example, include a bearing projection which seats in a receptacle of the spool bracket 5080B. The bearing projection and/or bracket receptacle may be formed of a low friction coefficient material such as PTFE.

The opposing spool flange 5020A may include a keyed receptacle 5082 which interfaces with a keyed projection 5102 of the tensioner assembly 5100. Alternatively, the tensioner assembly 5102 may include a keyed receptacle and the spool flange 5020A may include a keyed projection. In the example shown, the keyed projection 5102 is included on a base 5104 of the tensioner assembly 5100. In the example, the projection 5102 and receptacle 5082 have a hexagonal cross-section. In other embodiments, any other polygon, elongate round shape, obround, star shape, semi-circular, or other cross-section may be used. Due to the keyed interface between the spool 2696 and the tensioner assembly 5100, the spool 2696 may rotate in tandem with the base 5104 of the tensioner assembly 5100.

The exemplary tensioner assembly 5100 includes a bias member 5108. The bias member 5108 may be a coiled ribbon spring or constant force spring. A set anchors, such as a set of pins 5106A, B, may be coupled to the base 5104 and may capture a first end 5110 of the bias member 5108. The opposing end 5112 of the bias member 5108 may be routed or bent so as to generate a rounded end region 5114. The rounded end region 5114 may be teardrop shaped in certain examples.

The tensioner assembly 5100 may further include a cover body 5116 which is fixedly coupled to the spool bracket 5080A. The cover body 5116 includes a recess 5118 sized to accept the bias member 5108. The sidewalls of the recess 5118 are formed of a plurality of detents 5120 which the rounded end region 5114 of the bias member 5108 may seat in. As the spool 2696 rotates about its axis, the base 5104 of the tensioner assembly 5100 rotates with the spool 2696 while the cover body 5116 is stationary. Since the first end 5110 of the bias member 5108 is retained by the pins 5106A, B this may cause distortion of the bias member 5108. The bias member 5108 may exert a restoring force when distorted which applies tension through the base 5104 and spool 2696 to the chain 2670 of bags 26 (see, e.g., FIG. 37A). When the supply of consumable bags 26 in the dispenser 5000 is exhausted, the chain 2670 may be released from any grippers or retainers in the system 10. The bias member 5108 would then be allowed to return to a relaxed state. As the bias member 5108 transitions to the relaxed state, the spool 2696 would be rotated and the sacrificial tail 2683 of the chain 2670 would be wound back on to the spool 2696 or at least into the interior volume of the dispenser 5000. After the bias member 5108 is sufficiently relaxed, all remnants of the chain 2670 may be retracted into the dispenser 5000 and the removable cover assembly 5002 may be reinstalled.

The sidewall of the recess 5118 of the cover body 5116 may be formed of a series of detents 5120 to prevent excessive tension being built up by the tensioner assembly 5100. As a threshold tension force is breached, the bias member 5108 may relieve tension on the chain 2670 by slipping out of one detent 5120 and into an adjacent detent 5120. Thus, the tension generated by the tensioning assembly 5100 may be limited and may be maintained within a predetermined range dependent on the detent 5120 arrangement. The dispenser 5000 may be provided pre-tensioned to ensure outfeed from the dispenser 5000 is generally consistent from the first bag 26 of the chain 2670 to the end of the chain 2670.

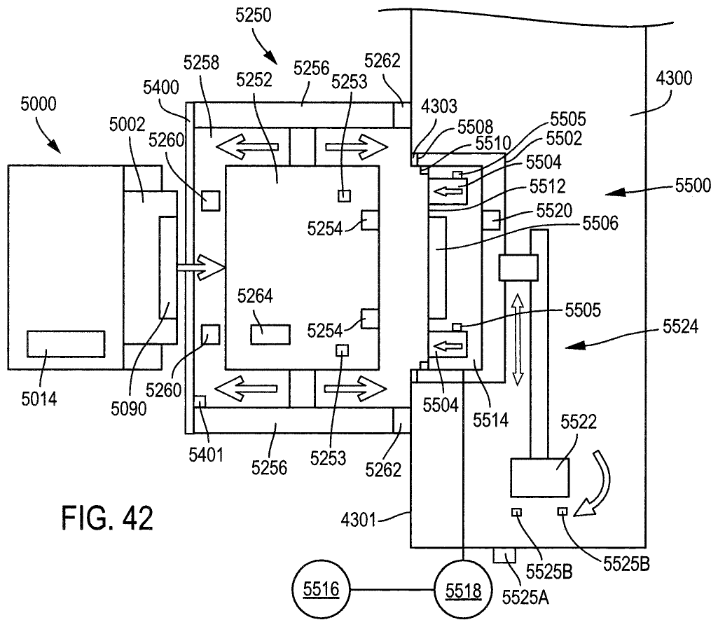

Referring now to FIG. 42, a block diagram of a portion of a docking assembly 5250 and a processing compartment 4300 with a sanitary interface assembly 5500 is depicted. To install a dispenser 5000 in a system 10, a user may place the dispenser 5000 in a dispenser carriage 5252 in a docking compartment 5258 of the docking assembly 5250. To do so, a docking assembly door 5400 (see also, e.g., FIG. 1) may be unlocked by the system 10 and opened by a user. The docking assembly door 5400 may include a lock assembly 5401 which includes built in lock latch state sensors and a door state sensor in data communication with the control system 15.

The carriage 5252 may include a set of retainers 5254. The retaining apertures of the dispenser 5000 may be engaged with the retainers 5254 to locate the dispenser 5000 within the carriage 5252. At least one lighting assembly 5260 including one or more light emitters (e.g. LEDs) may be included in the docking assembly compartment 5258. The lighting assembly 5260 may be powered when the door 5400 is in an open state to facilitate placement of the dispenser 5000 into engagement with the retainers 5254 (or removal of the dispenser 5000 from the carriage 5252). The control system 15 may monitor the door state sensor of the lock assembly 5401 to detect opening and closure of the door 5400.

The dispenser carriage 5252 may be coupled to one or more carriage actuator 5256. The control system 15 may govern displacement of the carriage 5252 within the docking compartment 5258 via commands to the carriage actuators 5256. At least one carriage position sensor 5262 may be included. For example, each carriage actuator 5256 may be associated with a position sensor 5262 which may output a data signal indicative of the position of the carriage 5252. In some embodiments, the carriage actuators 5256 may be leadscrew or ballscrew type actuators and the position sensors 5262 may be rotary encoders. Commands from the control system 15 to the carriage actuators 5256 may be based on the data signals received from any carriage position sensors 5262. The carriage 5252 may be displaced between a loading position (most distal the processing chamber 4300), a partially installed position, and a fully installed position in which a sealing member 5060 (see, e.g., FIG. 38A) of the dispenser 5000 is sealed against the exterior of the processing chamber 4300.

After a dispenser 5000 is loaded onto a carriage 5252, the door 5400 to the docking compartment 5258 may be closed and locked. The control system 15 may command displacement of the carriage 5252 to the partially installed position. In certain examples, the control system 15 may prohibit actuation of the carriage actuators 5256 until the lock assembly 5401 sensors indicate that the door 5400 is locked with the door 5400 in a closed state. The control system 15 may also inhibit actuation of the lock assembly 5401 to an unlocked state unless the data from the position sensors 5256 confirms the carriage 5258 is in the loading position. The carriage 5252 may include one or more dispenser detection sensors 5253. Any suitable sensor variety may be used, but certain implementations may employ limit switches which are actuated when a dispenser 5000 is present and properly positioned. Output from these sensors 5253 may be used to confirm a dispenser 5000 is installed in the carriage 5252 and properly seated.

An indicia reader 5264 may be included in the docking assembly 5250 and may read at least one indicium 5014 included on the dispenser 5000. The indicia reader 5264 may be a barcode reader, QR code reader, GS1 code reader, data matrix reader, bokode reader, RFID interrogator, NFC interrogator, or other variety of reader as appropriate for the indicia 5014 on the dispenser 5000. In certain examples, the indicia reader 5264 may be an imager. The control system 15 may receive data from the indicia reader 5264 and verify that the dispenser 5000 is acceptable for use. For example, the control system 15 may verify that a use by or expiration date determined from the indicium 5014 has not passed. The control system 15 may also check a unique identifier for the dispenser 5000 against a database (e.g. cloud database) to ensure that dispenser 5000 has not already been used, subject to recall, etc. The control system 15 may also set one or more operating parameter for the system 10 based on data obtained from the indicium 5014. For example, the control system 15 may determine a fill set point (e.g. volume) for each bag 26 based on information about the capacity of the bags 26 in the dispenser 5000 or the concentrate in the bags 26 in the dispenser 5000. Where multiple varieties of dispensers 5000 are compatible with a system 10, the type of dispenser 5000 may also be determined from data included in the indicia 5014.

In the event the dispenser 5000 is acceptable for use, a set of cover holders 5504 in the infeed aperture plug 5502 may be deployed against the removable cover assembly 5002 of the dispenser 5000. The cover holders 5504 may be air springs in certain examples and may be pressurized against the removable cover assembly 5002. In alternative examples, air spring type cover holders 5504 may be previously pressurized and the removable cover assembly 5002 may be driven into the cover holders 5504 as it is advanced toward the infeed aperture plug 5502 (e.g. may be in contact when or before the dispenser 5000 reaches the partially installed position). This may similarly hold the removable cover assembly 5002 in place via a bias exerted from the cover holders 5504. In still other examples, the removable cover assembly 5002 may be retained on the dispenser 5000 via an interference or friction fit. Magnets may also be used. One of the removable cover assembly 5002 and dispenser 5000 may include magnet(s) which attract respective magnet(s) or metallic bodies on the other of the removable cover assembly 5002 or dispenser 5000. The friction fit or magnetic coupling may hold the removable cover assembly 5002 in place when the lock bodies 5032A, B are disengaged. An adhesive coupling could also be utilized. In such embodiments, cover holders 5504 in the infeed aperture plug 5502 may be omitted.

A lock actuation assembly 5506 may also be included in the infeed aperture plug 5502. As the carriage 5252 is displaced to the partially installed position, the lock actuation assembly 5506 may engage the lock assembly 5090 of the dispenser 5000. With the removable cover assembly 5002 held in compression against the dispenser 5000 by the cover holders 5504 (or friction fit, magnetic coupling, adhesive, etc.), the lock actuation assembly 5506 may be powered to transition the lock assembly 5090 of the dispenser 5000 to a disengaged state. This may decouple the removable cover assembly 5002 from the dispenser 5000 while maintaining the seal between the dispenser 5000 and removable cover assembly 5002 with the cover holders 5504.

Still referring to FIG. 42, the infeed aperture plug 5502 may include a sealing member 5508 which forms a seal around the infeed aperture 4303 on the interior face of the wall 4301 of the processing compartment 4300. The infeed aperture plug 5502 may also include an exposed section spanning the infeed aperture 4303 which is in communication with the docking compartment 5258. A central region 5512 of the exposed section may be recessed. The carriage 5252 may be displaced to the fully installed position. As the carriage 5252 reaches the fully installed position, a sealing member 5060 of the dispenser 5000 may compress against the exterior of the processing compartment 4300 around the infeed aperture 4303. A perimeter region of the removable cover assembly 5002 (e.g. a face of the backing body 5034) may compress against a second sealing member 5510 of the infeed aperture plug 5502 at the perimeter of the exposed region of the infeed aperture plug 5502. In various examples, this establishes a sealed volume intermediate the removable cover assembly 5002 and the central region 5512 of the exposed section of the infeed aperture plug 5502. Surfaces of the removable cover assembly 5002 and infeed aperture plug 5502 exposed to the ambient environment may be isolated by the seal formed between the removable cover assembly 5002 and second sealing member 5510.

The central region 5512 may include one or more passages extending into an interior bay 5514 of the infeed aperture plug 5502. The interior bay 5514 may be in selective fluid communication with a vacuum source 5517 via a valve 5518. The valve 5518 may be opened to draw a vacuum within the sealed volume between the removable cover assembly 5002 and the infeed aperture plug 5502. The vacuum may serve to hold the removable cover assembly 5002 in sealing relationship with the second sealing member 5510 of the infeed aperture plug 5502. The vacuum may also be sufficient to overcome any frictional, magnetic, adhesive, etc. engagement of the removable cover assembly 5002 with the dispenser 5000 when the infeed aperture plug 5502 is displaced away from the dispenser 5000.

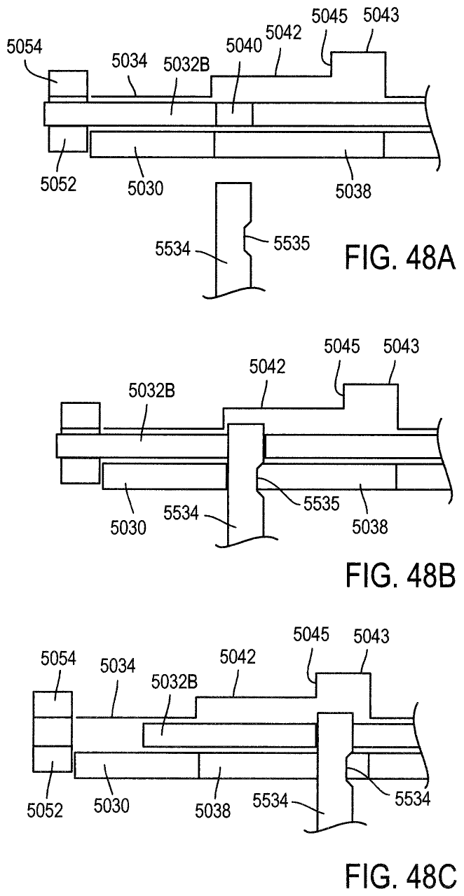

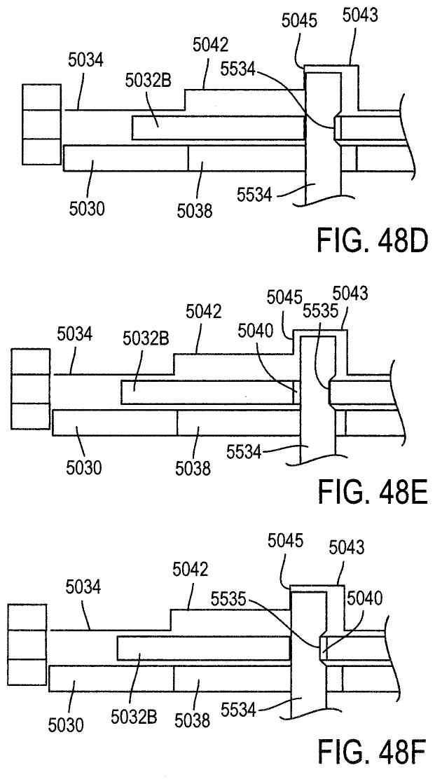

A pressure sensor 5520 may be included in the interior bay 5514 to verify an acceptable negative pressure has been established and maintained. The cover holders 5504 may be deactivated once the control system 15 determines a stable vacuum has been established. Alternatively, the cover holders 5504 may remain active and the vacuum established may be sufficient to retain the removable cover assembly 5002 in place despite the bias exerted by the cover holders 5504. In alternative examples, the lock actuation assembly 5506 may positively engage the removable cover assembly 5002 when the lock bodies 5032A, B are disengaged (see, e.g., FIGS. 48A-F. This may robustly retain the removable cover assembly 5002 in sealing engagement with the infeed aperture plug 5502. In such examples, the vacuum may be optional or may be weaker. A positive engagement between the lock actuation assembly 5506 and the removable cover assembly 5002 may also facilitate disengagement of a frictional, magnetic, adhesive, etc. type coupling between the removable cover assembly 5002 and the dispenser 5000.

With the removable cover assembly 5002 retained thereon, the infeed aperture plug 5502 may be displaced away from the infeed aperture 4303 via one or more plug displacement actuator 5522, 5524. In the example embodiment, a rotary actuator 5522 is included and may be powered to pivotally displace the infeed plug assembly 5502 relative to the infeed aperture 4303. A linear actuator 5524 is also included and may translationally displace the infeed aperture plug 5502. The actuators 5522, 5524 may be commanded by the control system 15 to drive the infeed aperture plug 5502 and removable cover assembly 5002 to a stowed position within the processing compartment 4300. In the stowed position, the infeed aperture plug 5502 and removable cover assembly 5002 are out of the way of any automation (e.g. a bag displacement assembly 2618) within the processing compartment 4300. The infeed aperture plug 5502 is shown displaced to a non-interfering position in FIG. 2 for instance. The rotary actuator 5522 and the linear actuator 5524 may each be associated with a brake or clamp which may be engaged to lock the actuators 5522, 5524. This may hold the infeed aperture plug 5502 in place when displacement is not desired.

One or more position sensor 5525A, B may be associated with each of the rotary actuator 5522 and linear actuator 5524. In some examples, the position of the infeed aperture plug 5502 within its linear displacement range may be sensed by a laser range finder type sensor. In such examples, this position sensor 5525A may be disposed on the exterior of the processing chamber 4300 with a line of sight to a portion of the removable cover assembly 5000 via a window in the processing chamber 4300. The rotary position of the infeed aperture plug 5502 may be sensed at the ends of its rotational range with one or more sensor(s) 5525B providing a binary output in certain examples (e.g. inductive sensors). A rotary encoder or potentiometer could be used in alternative embodiments. In certain embodiments, rotational displacement of the infeed aperture plug 5502 may be achieved by driving a crank arm with a linear actuator. The crank arm may depress switches or include a magnetic body which may alter the output of the rotation sensor(s) 5525B at the ends of its displacement range. The control system 15 may monitor the output signal from the respective position sensors 5525A, B to confirm the position of the infeed aperture plug 5502 and inform actuation commands to the rotary and linear actuators 5522, 5524.

Referring now to FIG. 43, a view of a docking assembly 5250 and infeed aperture plug 5502 in place in an infeed aperture 4303 to a processing compartment 4300 is depicted. As shown, the carriage 5252 includes a first and second support body 5272, 5272 for the dispenser 5000. Each of the support bodies 5270, 5272 is coupled to a respective carriage actuator 5256. The support bodies 5270, 5272 may include guide rails 5274 which ride along a set of bearings 5276 which are fixedly coupled in place within the docking assembly 5250. The support bodies 5270, 5272 may respectively accept a top and bottom portion of the dispenser 5000. The first support body 5270 may include a trough 5278 which may accept the flange 5008 of a dispenser 5000. The first support body 5270 may include at least one ramp feature 5280 extending toward the trough 5278. The ramp feature(s) 5280 may assist in directing the dispenser 5000 into position as the dispenser 5000 is loaded into the carriage 5252. A dispenser 5000 is shown in place within the example carriage 5252 in FIG. 44.

Referring now to FIGS. 43-44, the opposing second support body 5272 may include retainers 5254 which extend through the retaining apertures of a dispenser 5000 when the dispenser 5000 is installed. The retainers 5254 may be pins in some implementations. The pins may fit through a wide section of a respective pin retainer 5016 in a flange 5008 of a dispenser 5000. When the dispenser 5000 is released, the dispenser 5000 may displace or drop such that a narrow section of the pin retainers 5016 passes into a detent in the retainer 5254 pins. The head of the retainer 5254 pins may not pass through the narrow section of the pin retainers 5016. Thus, it may be necessary to lift the dispenser 5000 before decoupling it from the carriage 5252.

As shown, the infeed aperture plug 5502 includes an exposed central region 5512. The exposed central region 5512 may be formed by a housing section or panel 5536. Two cover holders 5504 are disposed extending through opposing end regions of the central region 5512. There are also a number of vacuum passages 5530 extending through the central region 5512. The vacuum passages 5530 may also provide clearance for features of the removable cover assembly 5002 when the removable cover assembly 5002 is adjacent the central region 5512. At the perimeter of the central region 5512 may be a raised ledge 5532 on which the second sealing member 5510 of the infeed aperture plug 5502 may be disposed. A rim 5538 surrounding and proud of the raised ledge 5532 may be present and the first sealing member 5508 may be associated therewith. As the infeed aperture plug 5502 is sealed against the surface of the interior wall of the processing compartment 4300 directly adjacent the infeed aperture 4303, the rim and first sealing member 5508 are not visible in FIG. 43. The central region 5512 additionally includes a number of slits 5532. Projections 5534 of the lock actuation assembly 5506 extend through the slits 5532.

Referring now also to FIG. 45, a view of an example infeed aperture plug 5502 with the housing panel 5536 removed is depicted (in some embodiments the housing panel 5536 may be entirely omitted). When the dispenser 5000 is displaced to the partially installed position by the carriage 5255, the projections 5534 may engage with the lock assembly 5090 of the dispenser 5000. Actuators 5540 within the interior bay 5514 of the infeed aperture plug 5502 may displace the projections 5534. In the example, the projections 5534 may extend from a pair of elongate bodies 5542A, B. The elongate bodies 5542A, B may each include or be outfitted with a set of guides 5544 which ride along bearings 5546 fixedly coupled to the interior bay 5514. Each elongate body 5542A, B may be driven by a dedicated pneumatic actuator 5540 in certain examples. In other examples, electromechanical actuators may be included. Where pneumatic actuators are used, pressure may be supplied to the pneumatic actuators 5540 to drive the elongate bodies 5542A, B toward and away from one another. The actuators 5540 may also have a neutral position in which they are unpressurized. The elongate bodies 5542A, B may remain in position with the pneumatic actuators 5540 in the neutral position. Where electromechanical actuators are used, position of the elongate bodies 5542A, B may be governed by the control system 15 based on sensor data from the actuators (e.g. encoder counts from motor encoders).

With reference to the example dispenser 5000 of FIGS. 34A-C, the projections 5534 may extend through respective overlay slits 5038 (see, e.g., FIG. 34C), through the aligned aperture 5040 in a lock body 5032A, B (see, e.g., FIG. 34C) and to a trough 5042 (see, e.g., FIG. 34C) of the backing body 5034 of the removable cover assembly 5002. Displacement of the elongate bodies 5542A, B and the projections 5534 extending therefrom may actuate the lock bodies 5032A, B as described in relation to FIGS. 34A-C. Thus, a removable cover assembly 5002 may be uncoupled and coupled to the dispenser 5000. With the lock bodies 5032A, B out of the way, the dispenser 5000 may then be advanced to a fully installed position. A sealing member 5060 on the dispenser 5000 may seal around infeed aperture 4303 when the dispenser 5000 reaches the fully installed position. The variable depth troughs 5042 may be deepest at a region aligned with the position of the projections 5534 when the lock bodies 5032A, B are disengaged. This may provide clearance for the projections 5534 as the dispenser 5000 is driven from the partially installed position to the fully installed position.

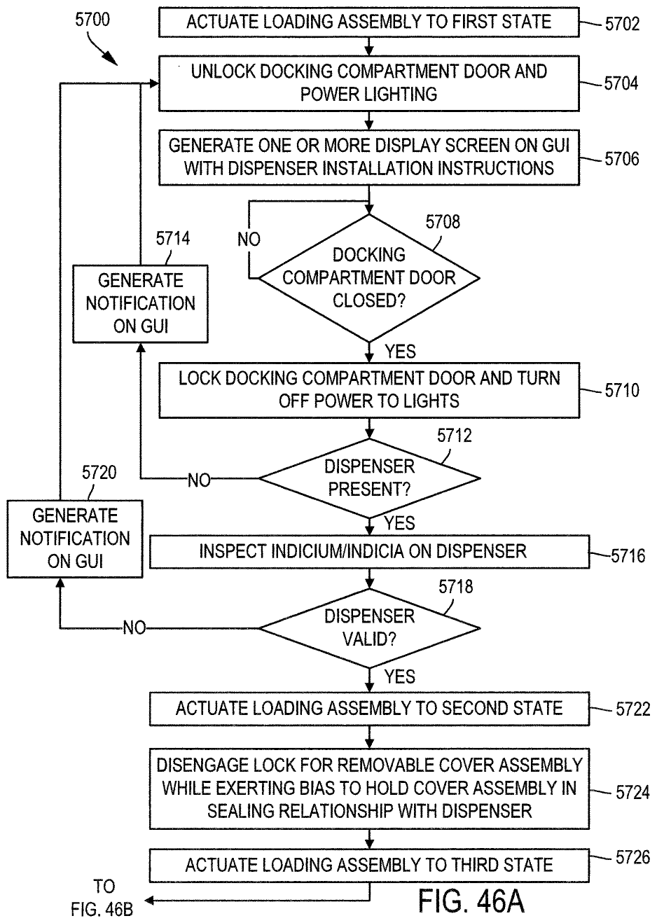

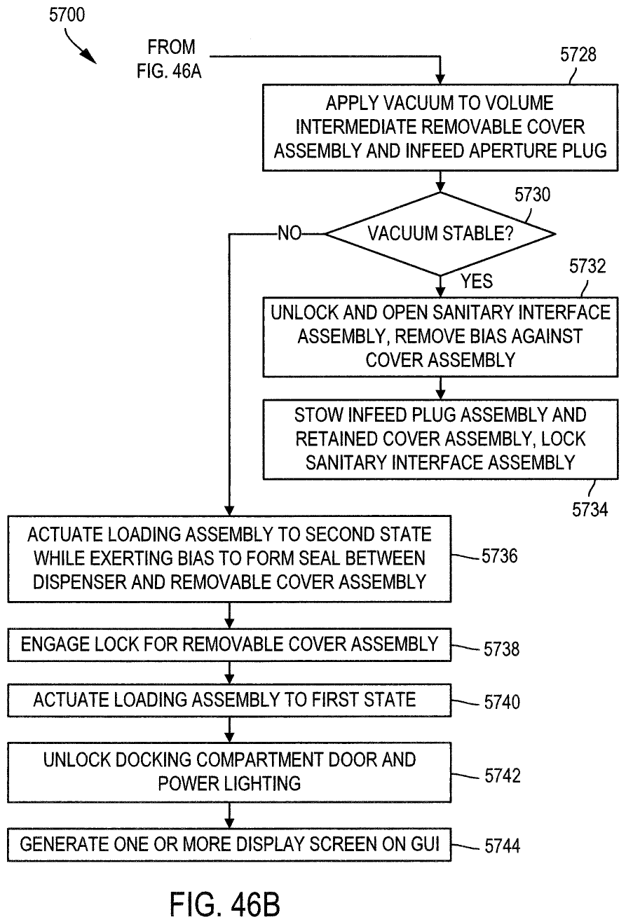

Referring now to FIGS. 46A-B, a flowchart 5700 is depicted illustrating a number of example actions which may be executed to load and access a dispenser 5000 with an example system 10. As shown, a carriage 5252 of a docking assembly 5250 may be actuated to a first state in block 5702. The first state may be a loading state in which the carriage 5252 is most distal the processing chamber 4300 of the system 10. In block 5704, the docking compartment door 5400 may be unlocked and any lighting assemblies 5260 in the docking assembly 5250 may be powered. In block 5706, the control system 15 may generate one or more screen for display on a graphical user interface 6100 of the system. The one or more screen may provide instructions in the form of text, images, illustrations, animations, or some combination thereof. The user may load the dispenser 5000 in accordance with the instructions on the graphical user interface 6100. When the docking compartment door 5400 is registered to be closed in block 5708, the control system 15 may actuate a lock for the door 5400 and terminate power to any lighting assemblies 5260 of the docking assembly 5250. A sensor such as a microswitch, magnetic sensor monitoring a metallic body in the door 5400, rotary potentiometer associated with a hinge of the door 5400, etc. may be provided to output a signal indicative of the position (e.g. closed, open, intermediate) of the door 5400. The position of the lock may also be monitored by a lock state sensor. In the event that control system 15 determines the output signal from the lock state sensor is indicative the lock is unlocked, the control system 15 may generate a notification. In some examples, all sensors relating to the position of the door 5400 and state of the lock may be included in a lock assembly 5401 (see, e.g., FIG. 42).

In some embodiments, at least one dispenser presence sensor 5253 (see, e.g., FIG. 42) may be included in the docking assembly 5250. The dispenser presence sensor(s) 5253 may, for instance, be a microswitch which is depressed when the dispenser 5000 is properly loaded in the carriage 5252. Optical sensors such as a beam break or reflectivity based sensor may be used. A load cell monitoring for the presence of a payload in the carriage 5252 may also be used. In some embodiments, the indicia reader 5264 may double as the dispenser presence sensor 5253. The control system 15 may monitor the data signal from the dispenser presence sensor(s) 5253 to determine if a dispenser 5000 has been loaded into the docking assembly 5250. If, in block 5712, a dispenser 5000 is determined to be absent (or misloaded), the control system 15 may generate a notification for display on the graphical user interface 6100 in block 5714 and the flowchart 5700 may return to block 5704. If a dispenser 5000 is detected in block 5712, the indicium 5014 on the dispenser 5000 may be inspected in block 5716. For example, the control system 15 may command an indicia reader 5264 to capture an image of the indicium 5014 and analyze the image. The control system 15 may cap the number of attempts to read the indicium 5014 at a predetermined number of retries and generate a notification in the event that the cap has been met. The control system 15 may also orchestrate actuation of the loading assembly to the first state and the notification may request the user reload the dispenser 5000 when the cap is exceeded.

The control system 15 may compare data gleaned from the indicium 5014 to a database to verify that the dispenser 5000 is valid or acceptable for use in block 5716. Various data on the indicium 5014 may be associated with a batch record for each bag 26 of fluid produced by the system 10. For example, a dispenser expiration date, serial number, lot number, number of bags 26 in the dispenser 5000, concentrate type in bags 26 within the dispenser 5000, etc. may be collected from the indicium 5014 and stored in the batch record created for each bag 26 in the dispenser 5000. The database may also be updated to indicate that the dispenser 5000 has been consumed.

If, in block 5718, the dispenser 5000 is determined to be invalid or unacceptable, the control system 15 may generate a notification for display on the graphical user interface 6100 in block 5720 and the flowchart 5700 may return to block 5704. If, in block 5718, the dispenser 5000 is valid, the loading assembly or carriage 5252 may be actuated to a second position in block 5722. The second position may be referred to elsewhere herein as a partially installed position. In the second position, the lock actuation assembly 5506 of the infeed aperture plug 5502 may be in engagement with the lock assembly 5090 of the dispenser 5000. The lock assembly 5090 of the dispenser 5000 may be actuated to a disengaged or unlocked state by the lock actuation assembly 5506 in block 5724. A bias may also be applied to the removable cover assembly 5002 of the dispenser 5000 to hold the removable cover assembly 5002 in a sealing relationship with the dispenser 5000 in block 5724. This bias may be exerted via a bias member (e.g. a cover holder 5504 such as one or more air spring) included in the infeed aperture plug 5502. The output signal from a bias member sensor 5505 (see, e.g. FIG. 42) may be analyzed to confirm bias is being applied to the removable cover assembly 5002. Where the bias is exerted by one or more air spring, a position sensor (e.g. a magnetic or Hall sensor monitoring a displaceable cylinder of each air spring) signal output may be checked to confirm force is being applied to the removable cover assembly 5002. The control system 15 may not command actuation of the lock assembly 5090 to the unlocked state absent a signal indicative that bias force is being applied to the removable cover assembly 5002. One or more retry may be performed (up to a preset cap) in the event such a signal is not detected. The carriage 5522 may be displaced away from and back to the second position each retry. As mentioned above, the bias may be optional if the removable cover assembly 5002 is otherwise (e.g. frictionally, magnetically, adhesively) held in place on the dispenser 5000.

The carriage 5252 may be displaced to a third position in block 5726. A sealing member 5060 of the dispenser 5000 may establish a sealing relationship around the infeed aperture 4303 in the third position. The periphery of the removable cover assembly 5002 may also form a fluid tight seal against a gasket or sealing member disposed at the periphery of the exposed surface of the infeed aperture plug 5502. In some embodiments, the bias member sensor (e.g. magnetic sensors monitoring the position of the cylinders of any air springs) output signal may be analyzed by the control system 15 to verify displacement to the third position has occurred. In block 5728, vacuum may be applied to the sealed volume intermediate the removable cover assembly 5002 and the infeed aperture plug 5502.

As mentioned elsewhere, vacuum may be optional if the removable cover assembly 5002 is brought into positive engagement with the infeed aperture plug 5502 before being doffed from the dispenser 5000. For example, the actuators 5540 of the infeed aperture plug 5502 may be powered to drive the projections 5534 of the lock actuation assembly 5506 further in the disengaging direction in block 5728 (instead or in addition to applying vacuum). This may drive a detent or notch 5535 on the projections 5534 into engagement with respective portions of the removable cover assembly 5002 as further described in relation to FIGS. 48A-F.

The control system 15 may monitor data from one or more pressure sensor 5520 in fluid communication with the sealed volume to determine whether the pressure in the sealed volume is at least below a predefined set point. The control system 15 may also monitor the pressure to ensure that the decay rate of pressure in the sealed volume is below a predefined threshold. If, in block 5730, a stable vacuum has been established, the sanitary interface assembly 5500 may be unlocked and opened in block 5732. Alternatively, if a vacuum is not utilized and the removable cover assembly 5002 is determined to be positively engaged with the lock actuation assembly 5506, the sanitary interface assembly 5500 may be unlocked and opened in block 5732. The bias against the removable cover assembly 5002 may also be alleviated in block 5732. This may, for instance, be accomplished by depressurizing the air springs. Alternatively, the bias may remain present and the vacuum (or positive engagement between the removable cover assembly 5002 and the infeed aperture plug 5502) may be sufficient to retain the removable cover assembly 5002 in place in spite of the bias. In block 5734, the infeed aperture plug 5502 and the vacuum retained removable cover assembly 5002 may be displaced to a stowed position. The infeed aperture plug 5502 may also be unlocked in block 5734. A locking clamp for a pneumatic cylinder (e.g. linear actuator 5524) may be disengaged and/or a brake for an electromechanical actuator (e.g. rotary actuator 5522) may be disengaged. Once in the stowed position, the sanitary interface assembly 5500 may be locked (e.g. any clamps and/or brakes on actuation components may be engaged). The data signal(s) from one or more infeed aperture plug position sensor may be checked to verify that the infeed aperture plug 5502 is in the stowed position. In the event that signal(s) from the infeed aperture plug position sensor(s) indicate that the infeed aperture plug 5502 is not in the stowed position, a notification may be generated by the control system 15 (e.g. a screen may be displayed on a user interface 6100).

If, in block 5730, a stable vacuum is not established, the carriage 5252 may be actuated to the second position while the bias compressing the removable cover assembly 5002 against the dispenser 5000 is exerted in block 5736. In block 5738, the lock assembly 5090 of the dispenser 5090 may be re-engaged or returned to a locked state by the lock actuation assembly 5506 of the infeed plug assembly 5502. The infeed plug assembly 5502 may also be locked in place. The carriage 5252 may then be displaced to the first position in block 5740. The docking compartment door 5400 may be unlocked and any lighting assemblies 5260 of the docking assembly 5250 may be powered in block 5742. In block 5744, one or more screens may be generated for display on the graphical user interface 6100 by the control system 15. The screens may notify the user that the dispenser 5000 could not be opened and present troubleshooting information. Alternatively, the graphical user interface 6100 may display a screen requesting the dispenser 5000 be replaced with a different dispenser 5000. In alternative examples, flowchart 5700 may pass from block 5738 to block 5724 for a capped number of retries. If a stable vacuum cannot be established after the available number of retries has been exhausted, the flowchart 5700 may proceed from block 5738 to block 5740.

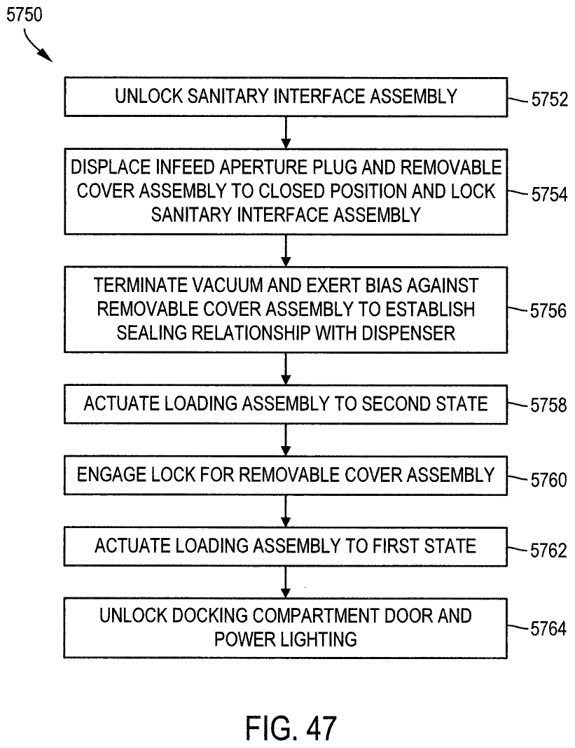

Referring now to FIG. 47, a flowchart 5750 detailing a number of example actions which may be executed to unload a dispenser 5000 from a system 10 is depicted. As shown, the sanitary interface assembly 5500 of the system 10 may be unlocked in block 5752. In block 5754, the infeed aperture plug 5502 and removable cover assembly 5002 of the to-be-removed dispenser 5000 may be displaced to a closed or occluding position and the sanitary interface assembly 5500 may be locked in place. The closed position may be a position in which the infeed aperture plug 5502 is sealed around the infeed aperture 4303 (see, e.g. FIG. 43). In block 5756, a bias pressing the removable cover assembly 5002 against the dispenser 5000 may be applied and application of vacuum to the sealed volume between the infeed aperture plug 5502 and removable cover assembly 5002 may be terminated. Where the removable cover assembly 5002 is positively retained on the lock actuation assembly 5506, the removable cover assembly 5002 may be released in block 5756 (see, e.g., progression of FIGS. 48E-F). The carriage 5252 may be actuated to the second position in block 5758. In block 5760, the lock assembly 5090 of the dispenser 5000 may be transitioned to a locked state. The bias against the removable cover member 5002 may also be removed in block 5760 as the removable cover assembly 5002 will seal the interior volume of the dispenser 5000 when the lock assembly 5090 is in a locked state. Alternatively, a frictional, magnetic, or adhesive engagement may be formed to hold the removable cover assembly 5002 in place and the bias may not be applied. In block 5762, the carriage 5252 may be displaced to the first state. The docking compartment door 5400 may be unlocked and any lighting assemblies 5260 of the docking assembly 5250 may be powered in block 5764. The dispenser 5000 may then be accessed and removed by a user.

Referring now primarily to FIGS. 48A-F, a progression of block diagrams showing a removable cover assembly 5002 being unlocked and positively retained by projections 5534 of a lock actuation assembly 5506 are depicted. The remainder of the dispenser 5000 (with the exception of the rim 5054) is hidden for sake of simplicity. Additionally, only one projection 5534 interfacing with a portion of one lock body 5032B is depicted. A number of projections 5534 may interface with respective portions of a given lock body 5032B in the same manner as illustrated in FIGS. 48A-F. Additionally, the opposing lock body 5032A (see, e.g., FIG. 34C) would interface with projections 5534 in the same manner, however, the displacement of the components would be in the opposing direction (symmetric about the midplane of the removable cover assembly 5002).

As shown, in an initial position (e.g. just after a dispenser 5000 has been loaded by a user), the removable cover assembly 5002 may be spaced from the projections 5534 of the infeed aperture plug 5502 (see, e.g., FIG. 42). The lock bodies 5032A, B may be engaged with bolt retainers 5052 of the rim 5054 of the dispenser 5000 locking the removable cover assembly 5002 in sealing relation with the dispenser 5000. As the dispenser 5000 is displaced toward the processing chamber 4300, the dispenser 5000 may reach a partially installed position as shown in the FIG. 48B. In the partially installed position, the projections 5534 may extend at least partially into respective apertures 5040 in the lock bodies 5032A, B. In the example, the projections 5534 extend through the apertures 5040 and partially into the variable depth troughs 5042 of the backing body 5034 of the removable cover assembly 5002.

Referring now to FIG. 48C, to unlock the removable cover assembly 5002 from the dispenser 5000, actuators 5540 (see, e.g., FIG. 45) of the infeed aperture plug 5502 may be pressurized (or otherwise powered if not pneumatic) to displace the projections 5534. As the projections 5534 extend through the apertures 5040, this may drive the lock bodies 5032A, B to displace out of engagement with the bolt retainers 5052 of the rim 5054 of the dispenser 5000. The lock bodies 5032A, B may hit a stop which prevents further displacement of the lock bodies 5032A, B in the unlocking direction. Where pneumatic actuators 5540 are used, this stop may be encountered before the end of the displacement range afforded by the actuators 5540. The pneumatic actuators 5540 may be put in a neutral or unpressurized state after the lock bodies 5032A, B have reached an end of their displacement range in the unlocking direction. The projections 5534 may be in line with a pocket 5043 of the variable depth trough 5042 in the backing body 5034 of the removable cover assembly 5002 as shown in FIG. 48C. As described elsewhere herein, a bias may be applied to the removable cover assembly 5002 to maintain it in place on the dispenser 5000 if it is not otherwise held in place.

After the removable cover assembly 5002 is unlocked, the dispenser 5000 may be driven to a fully installed position as shown in FIG. 48D. The projections 5534 may extend into pockets 5043 of their respective variable depth troughs 5042 in the backing body 5034. The projections 5534 may include detents 5535 sized to accept a portion of the removable cover assembly 5002. In the example shown, the detent 5535 is sized to accept the thickness of a lock body 5032A, B. When the dispenser 5000 is advanced to the fully installed position, the detents 5535 may be aligned with the sidewalls of the apertures 5040 through which the projections 5534 extend. Referring now to FIG. 48E, to positively engage the removable cover assembly 5002, the actuators 5540 may be pressurized (or otherwise powered if not pneumatic) to drive the projections 5534 further in the unlocking direction. This may drive the detents 5535 against the sidewalls of the respective apertures 5040 such that a portion of the projections 5534 overhang the front and rear faces of the lock bodies 5032A, B. The detents 5535 may thus present an interference to inadvertent release of the removable cover assembly 5002 from the projections 5534 or jostling relative thereto. Once in this position, the infeed aperture plug 5502 may be brought to a stowed position removing the removable cover assembly 5002 from the dispenser 5000 in the process. As the lock bodies 5032A, B are captured in the detents 5535, the removable cover assembly 5002 may be robustly held in place on the infeed aperture plug 5502.

Referring now to FIG. 48F, when the removable cover assembly 5002 is reinstalled on a dispenser 5000, the infeed aperture plug 5502 may be brought into a sealing relationship with the wall of the processing chamber 4300 (see, e.g., FIG. 43). Where pneumatic actuators 5540 are used, the actuators 5540 may be powered to drive the projections 5534 in a locking direction. The projections 5534 may hit a stop wall 5045 formed by the pocket 5043 of the variable depth trough 5042 of the backing body 5034. This may free the lock bodies 5032A, B from the detents 5535 without resulting in substantial movement of the lock bodies 5032A, B toward the locked position. The lock bodies 5032A, B may be re-engaged with the bolt retainers 5052 of the rim 5054 of the dispenser 5000 by displacing the dispenser 5000 to its position in FIG. 48C and powering the actuators 5540 to drive the projections 5534 to the position shown in FIG. 48B. With the removable cover assembly 5002 reinstalled on the dispenser 5000, the dispenser 5000 may then be removed from the system 10.

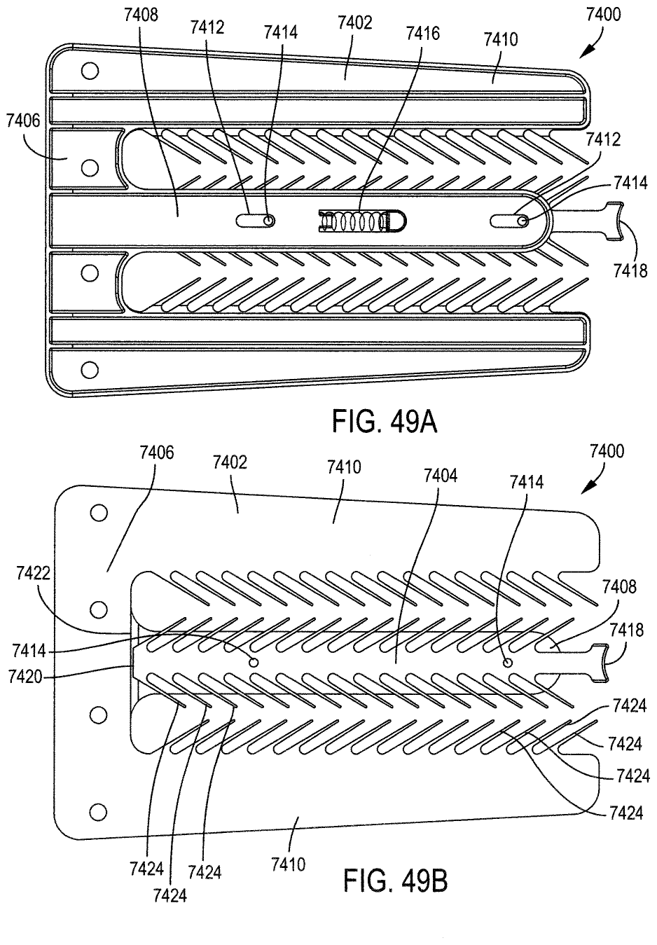

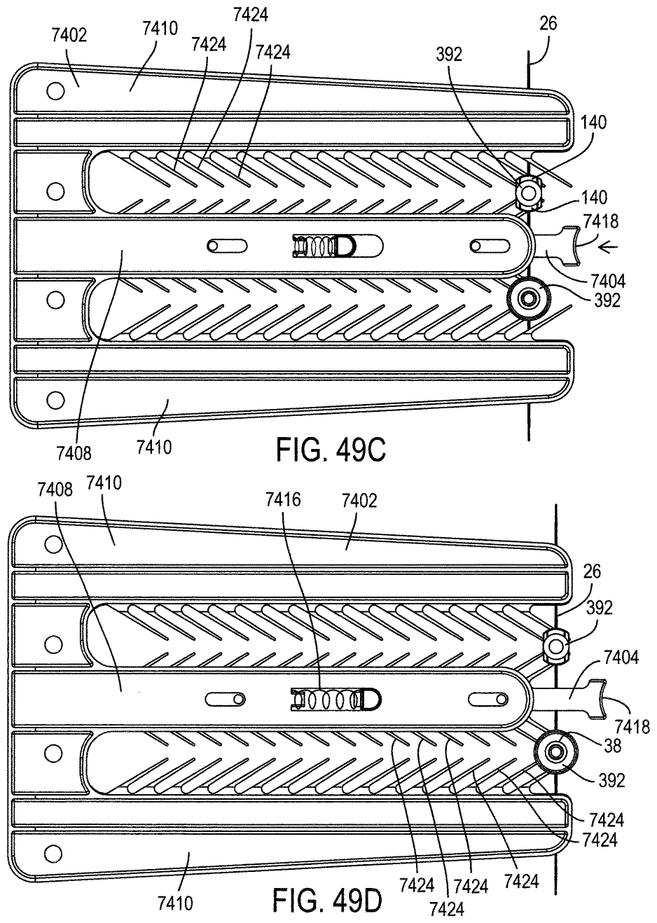

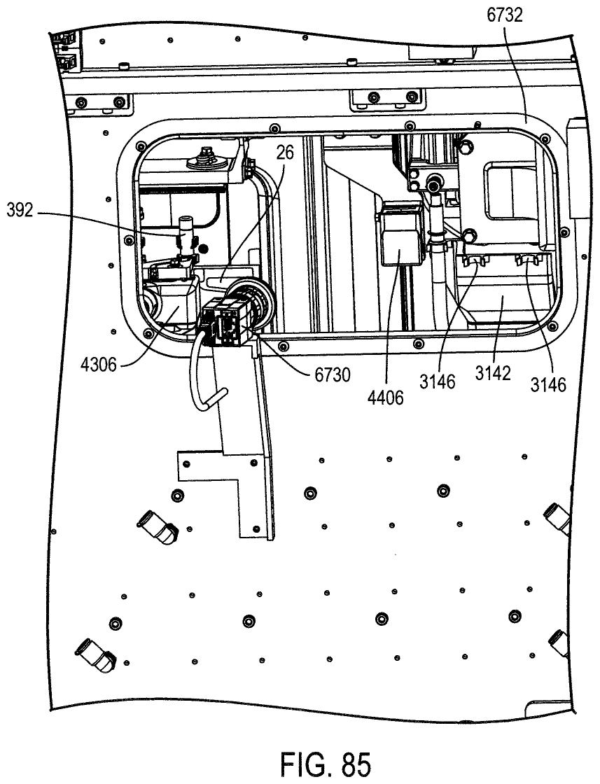

Referring now to FIG. 49A, in certain examples a system 10 may accept bags 26 which are included in a chain 2670 (or otherwise coupled to one another) as well as discrete bags 26 which are separate from one another. Alternatively, a chain 2670 of bags 26 may not be utilized in certain systems 10. A system 10 may accept multiple varieties of dispensers 5000. A first accepted dispenser 5000 variety may house bags 2670 on a chain 2670 which are included on a spool 2696 (see, e.g., FIG. 37B) and a second accepted dispenser 5000 may include bags 26 which are housed individually. Where a system 10 only accepts dispensers 5000 of individual bags 26, certain components of the system 10 may be absent. For example, a singulation assembly 4400 (see, e.g. FIG. 74) and a bag chain orientation sensor 6730 (see, e.g., FIG. 85) are preferably absent.

As shown in FIGS. 49A-B, where a dispenser 5000 contains a plurality of isolated bags 26, the dispenser 5000 may include a bag vending assembly 7400. The example bag vending assembly 7400 shown in FIG. 49A includes a main or first portion 7402 and a comb or second portion 7404. The first portion 7402 may remain static and may be mounted to a portion of the dispenser 5000 such as housing portion 5006B (see, e.g., FIG. 34C). The first portion 7404 may include a crosspiece 7406 including a central cantilevered projection 7408 and lateral cantilevered projections 7410 extending from each end region of the crosspiece 7406. Each of the central cantilevered projection 7408 and lateral cantilevered projections 7410 may extend parallel to one another and perpendicular to the axis of the crosspiece 7406. Various ribs or other strengthening features may be included on the crosspiece and cantilevered projections 7408, 7410 to inhibit deflection of the members during operation of the vending assembly 7400.

The comb or second portion 7404 may be coupled to the main portion 7404 and may be displaceable relative to the main portion 7404. As shown, the main portion 7404 may include one or more guide 7412 for the comb portion 7404. In the example, the guides 7412 are depicted as slots in the central cantilevered projection 7408. The comb portion 7404 may be coupled to the main portion 7402 via guide pins 7414 which mate into the comb portion 7404 through the guides 7412. Other guiding interfaces are possible. For example, some embodiments may include cooperating dovetail or other rail features in the central cantilevered projection 7408 and comb portion 7404. The comb portion 7404 may be biased to a first end of its displacement range by one or more bias member 7416 (e.g. a compression spring). The comb portion 7404 may include a contact surface 7418 at a first terminal end of the comb portion 7404 which serves as an upstream displacement assembly 4302 (see, e.g. FIG. 68A) interface. The opposing second terminal end of the comb portion 7404 may be a stop surface 7420 which may contact a wall 7422 of the cross piece 7406 to inhibit travel of the comb portion 7404 to define a second end of the comb portion's 7404 displacement range.

Referring now also to FIGS. 49C-D, each of the first and second portion 7402, 7404 may include a complimentary number of bag retainers 7424. The face of each of the lateral cantilevered projections 7410 most proximate the central cantilevered projection 7408 may include a desired number of bag retainers 7424. Complimentarily, the faces of the comb portion 7404 most proximal the lateral cantilevered projections 7410 may include bag retainers 7424. The number shown is merely exemplary and bag vending assemblies 7400 with a greater or lesser number of bag retainer 7424 sets and thus differing bag 26 capacities are possible. In the example embodiment, the bag retainers 7424 are flexures such as fingers or tines. The bag retainers 7424 are at an angle to the lateral cantilevered projections 7410 and the axial dimension of the comb portion 7404. In the example, the bag retainers 7424 extend at an oblique angle to the long axis of the lateral cantilevered projections 7410 and comb portion 7404. In certain examples the bag retainers 7424 may extend at an angle of 140°-160° (e.g. substantially 150°). Ports 392 of bags 26 may be captured in between the bag retainers 7424 of the main portion 7402 and the comb portion 7404. Only one bag 26 is shown in FIGS. 49C-D though the number of bags 26 may equal the number of sets of bag retainers 7424. As the comb portion 7404 is driven toward the second end of its displacement range, the flexure type bag retainers 7424 on the comb portion 7404 may elastically deflect around respective ports 392 held in the bag vending assembly 7400. The bias member 7416 may also enter a stressed state as this occurs. The angle of the bag retainers 7424 of the comb portion 7404 may assist in providing a ramped or sloped face which facilitates deflection of the comb portion 7404 bag retainers 7424 around the ports 392 as the comb portion 7404 is displaced toward the second end of its displacement range. When the comb portion 7404 is displaced toward the second end of its displacement range, the bag retainer 7424 of the comb portion 7404 previously on a first or downstream side of a given port 392 may be displaced to an opposing or upstream side of the port 392 (see, e.g., FIG. 49C). The flexure type bag retainers 7424 may also relax to an undeflected state. The bag retainers 7424 of the lateral cantilevered projections 7410 may resist displacement of the port 392 maintaining the position of the port 392 during displacement of the comb portion 7404.

When force on the comb portion 7404 is released, the bias member 7416 may relax from the stressed state toward a more relaxed state. In turn, this will drive the comb portion 7404 toward the first end of its displacement range. The bag retainers 7424 on the comb portion 7404 may press into the upstream side of the port 392. The angle of the bag retainers 7424 on the lateral cantilevered projections 7410 may facilitate deflection of these bag retainers 7424 and allow the port 392 to advance in a downstream direction as the comb portion 7404 is advanced toward the first end of its displacement range. Thus each displacement of the comb portion 7404 toward the second end of its displacement range and back will displace all bags 26 in the bag vending assembly 7400 one set of bag retainers 7424 in the forward or in upstream direction. The lead bag 26 (upstream most) would be ejected from the bag vending assembly 7400 as the comb portion 7404 is returned to its resting position under the force exerted by the bias member 7416. Typically, this bag 26 would be held by a bag displacement assembly 2618 and ejection of the bag 26 would free the bag 26 for downstream filling and other processing. Thus, the downstream most bag retainers 7424 may behave as a vending output for the bag vending assembly 7400.

Where ports 392 include ribs 140 or an attachment that includes projections such as ribs 140, the ribs 140 (or other protrusions) may be positioned above the bag retainers 7424. The lower set of ribs 140 (see, e.g., FIG. 21A-F) may rest on the top of the bag retainers 7424. The same may be true for other bag vending assembly 7400 embodiments described herein. Attachments such as any of the projection bearing bodies 142 shown in FIGS. 10A-B, 16A-18C, the ribbing clip 168 of FIG. 13, the ribbed rings 180 of FIGS. 14A-C, or the ribbed spacers 141 of FIGS. 15A-B may also provide rigid surfaces surrounding flexible ports 392. The rigid surfaces may interface with a bag vending assembly 7400 and may assist in feeding of bags 26 within example bag vending assemblies 7400.

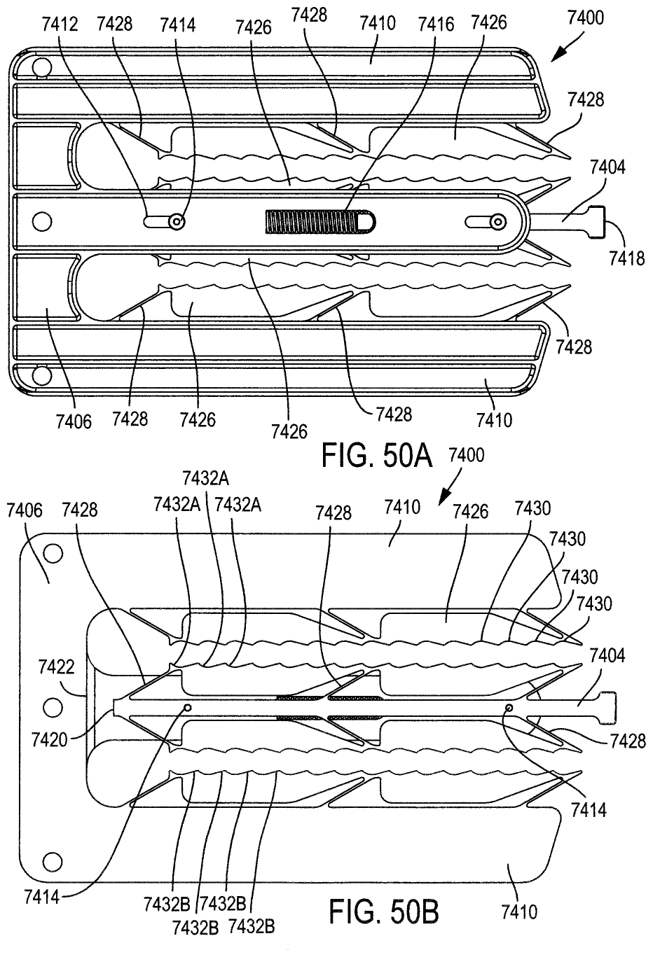

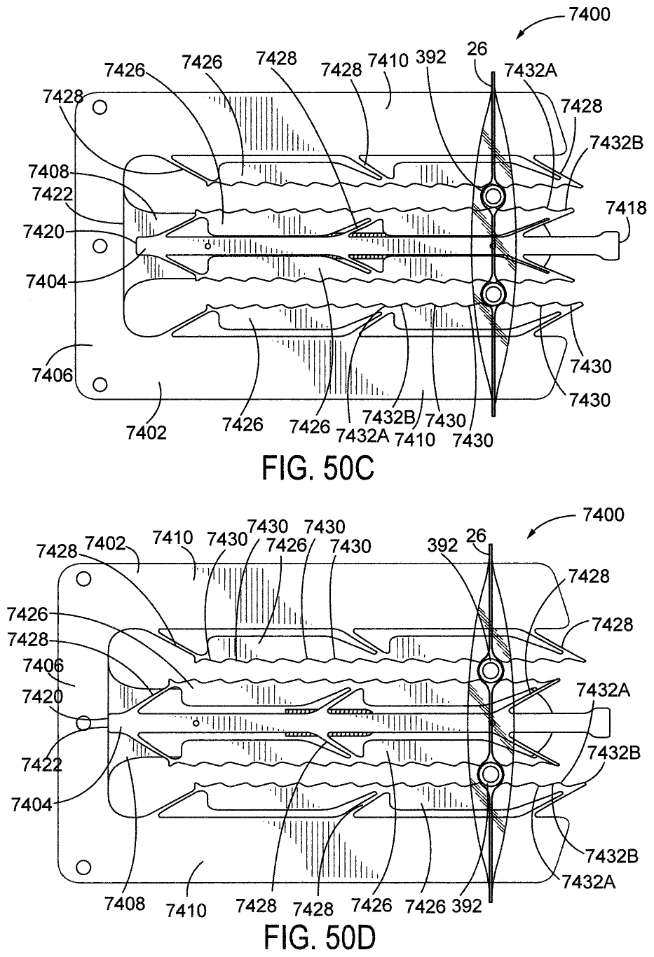

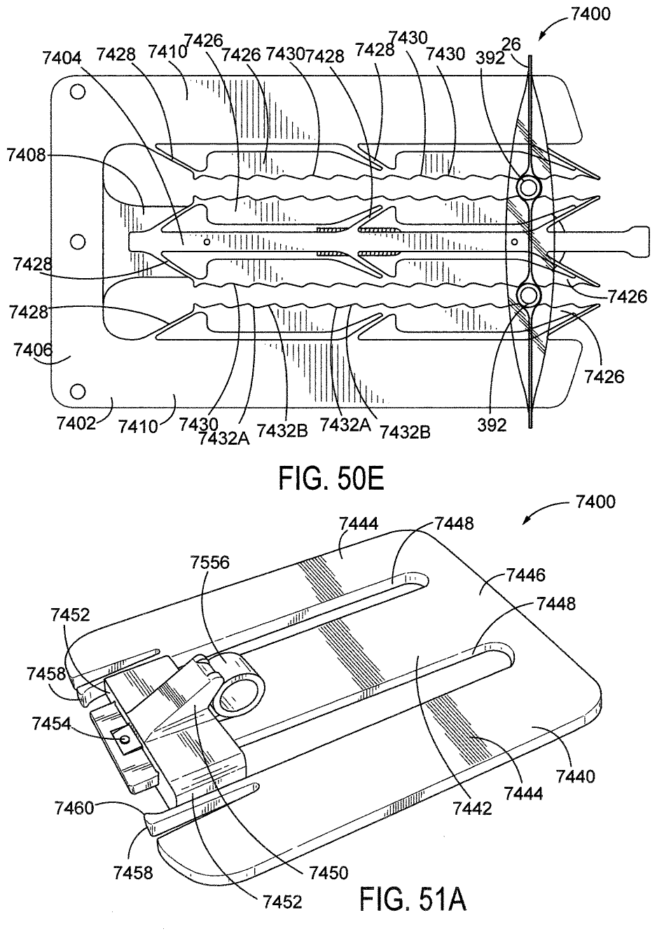

Referring now to FIGS. 50A-B, in certain examples, the bag retainers 7424 on each lateral cantilevered projection 7410 and each side of the comb portion 7404 may be amalgamated into ratcheting members 7426. Each ratcheting member 7426 may be connected to a respective lateral cantilevered projection 7410 or side of the comb portion 7404. The ratcheting members 7426 may be displaceable relative to the respective lateral cantilevered projection 7410 or side of the comb portion 7404 to which they are coupled. In the example shown, the ratcheting members 7426 are formed integrally with the lateral cantilevered projections 7410 or comb portion 7404. As shown, a number of flexures 7428 couple the ratcheting members 7426 to the respective lateral cantilevered projection 7410 or side of the comb portion 7404. Distortion of the flexures 7428 permits displacement of the ratcheting members 7426 relative to the respective lateral cantilevered projection 7410 or side of the comb portion 7404.

As shown, the faces of the ratcheting members 7426 on the lateral cantilevered projections 7410 most proximal the comb portion include a series of detents 7430. Complimentarily the faces of the ratcheting members 7426 of the comb portion 7404 most proximate the lateral cantilevered projections 7410 may have a series of detents 7430 as well. The number of sets of detents 7430 shown in the example is merely exemplary, any number of sets of detents 7430 may be included in alternative embodiments to change the bag 26 capacity for a bag vending assembly 7400.

Each detent 7430 includes an upstream portion 7432A and a downstream portion 7432B which are sloped in opposite directions. The downstream portion 7432A of each detent 7430 may be sloped in a shallower manner than the upstream portion 7432B of each detent. The term sloped in relation to the detents 7430 encompasses both flat ramped features and curved features (as shown in FIGS. 50A-B). The detents 7430 on the ratcheting members 7426 of the lateral cantilevered projections 7410 are mirrored relative to the detents 7430 on the most proximate ratcheting member 7426 of the comb portion 7404. The gap between the ratcheting member 7426 on a lateral cantilevered projection and the nearest ratcheting member 7426 of the comb portion 7404 may be widest where the upstream and downstream portions 7432A, B of a given set of corresponding detents 7430 meet. This gap may be narrowest at a transition region between sets of adjacent detents 7430 on the ratcheting members 7426. In the example, the gap is narrowest where the upstream portions 7432A of a first set of detents 7430 meets the downstream portion 7432B of the adjacent downstream set of detents 7430. At its narrowest, the gap is smaller than the diameter of a port 392 of a bag 26. At its widest, the gap may be slightly smaller than the diameter of the port 392 of a bag 26 such that the flexures 7428 are partially distorted when a bag 26 is present. This may lead the flexures 7428 to apply some compressive holding force against the ports 392.

Referring now also to FIGS. 50C-E, the comb portion 7404 may be displaced toward one end of its displacement range and returned under the urging of at least one bias member 7416 to advance bags 26 through the bag vending assembly 7400. As the comb portion 7404 begins to displace toward the second end of its displacement range, the downstream portions 7432B of the detents 7430 in the comb portion 7404 may slide over the ports 392 held in the bag vending assembly 7400. As this occurs, the flexures 7428 coupling the ratcheting members 7426 of the comb portion 7404 to the base of the comb portion 7404 may distort to allow the ratcheting members 7426 to displace toward the base of the comb portion 7404 (see, e.g., FIG. 50C). As the stop surface 7420 reaches the wall 7422 of the crosspiece 7406, the adjacent upstream detent 7430 on the comb portion 7404 may have displaced to a position where it is cradling the port 392. The comb portion 7404 is displayed in this position in FIG. 50D. As shown, the flexures 7428 coupling the ratcheting member 7426 of the comb portion 7404 to the base of the comb portion 7404 may substantially relax to a non-distorted state as the comb portion 7404 reaches the second end of its displacement range.

Referring now primarily to FIG. 50E, as the comb portion 7404 is released, the bias member 7416 may drive the comb portion 7404 toward the first end of its displacement range. The sharper sloped portion of each detent 7430 on the comb portion may press against the upstream side of any respective port 392 as the comb portion 7404 is displaced toward the first end of the displacement range. This may drive the ports 392 along the shallow sloped downstream portions 7432B of the detents 7430 in the opposing ratcheting members 7426 extending from the lateral cantilevered projections 7410. As this occurs, the flexures 7428 coupling the ratcheting members 7426 of the lateral cantilevered projections 7410 to the remainder of the lateral cantilevered projections 7410 may distort. This may permit the ratcheting members 7426 of the lateral cantilevered projections 7410 to displace toward the remainder of the respective lateral cantilevered projections 7410. As the ports 392 are displaced into the adjacent downstream detents 7430 of the ratcheting members 7426 of the lateral cantilevered projections 7410, the flexures 7428 may restore to a less distorted state. Thus, the ports 392 for each bag 26 may be indexed in the downstream direction in the bag vending assembly 7400. The foremost bag 26 may be ejected from the ratcheting members 7426. Thus, the last set of detents 7430 may behave as a vending output for the bag vending assembly 7400.

Still referring to FIGS. 50A-E, the flexures 7428 of example vending assembly 7400 are arranged such that the force require to depress the comb portion 7404 to the second end of its displacement range is largely untethered to the capacity of the bag vending assembly 7400. The number of flexure 7428 may be kept minimal such that the force require to depress the comb portion 7404 is closer to the force required to distort the bias member 7416 when compared to, for example, the embodiment shown in FIGS. 49A-D. Thus, the bag vending assembly 7400 depicted in FIGS. 50A-E may provide more latitude in selection of bag displacement assembly 2618 hardware.

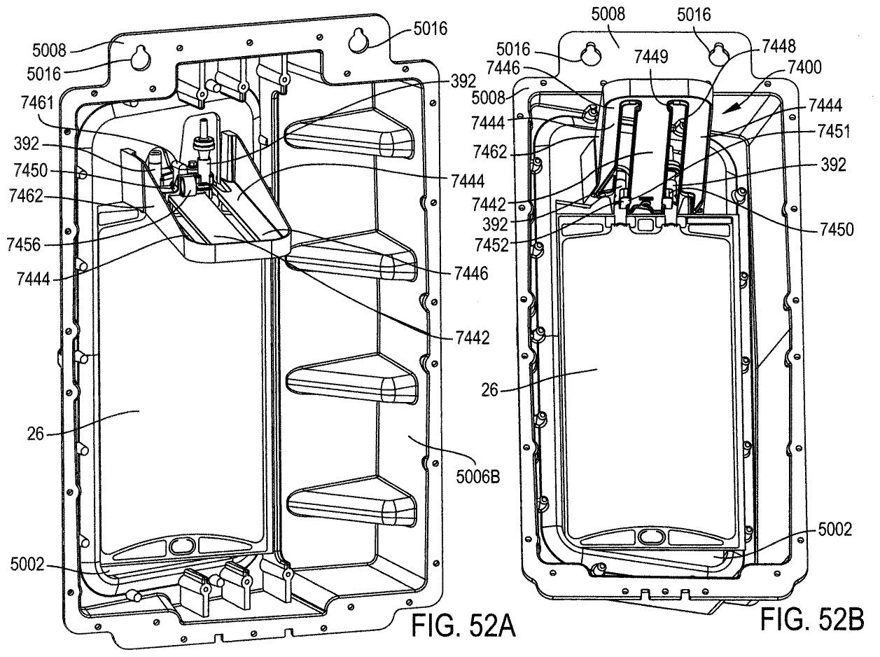

Referring now to FIGS. 51A-51C, another example bag vending assembly 7400 is depicted. As shown, the bag vending assembly 7400 may include a main body 7440. The main body 7440 may include a central cantilevered projection 7442 and a set of lateral cantilevered projections 7444 flanking the central cantilevered projection 7442. Each of the cantilevered projections 7442, 7444 may extend from a crosspiece 7446. In the example, each of the cantilevered projections 7442, 7444 are parallel to one another and each extend in a direction substantially perpendicular to the crosspiece 7446. Various ribs 7462 may be included to provide additional rigidity to the main body 7440 (see, e.g., FIG. 52A).

Channels 7448 are present between the central cantilevered projection 7442 and the flanking lateral cantilevered projections 7444. The channels 7448 may have a width which is about equal to the diameter of ports 392 of bags 26 to be held in the bag vending assembly 7400. Any ribs 140 on the ports 392 may overhang the channels 7448 and rest upon the cantilevered projections 7442, 7444. The depth of the channels 7448 may dictate the number of bags 26 which may be stored in a bag vending assembly 7400. The bag vending assembly 7400 shown in FIGS. 51A-51C is merely exemplary and the length of the cantilevered projections 7442, 7444 may be adjusted in other embodiments to alter the capacity of the bag vending assembly 7400 as desired.

A follower 7450 may be coupled to the central cantilevered projection 7442. The central cantilevered projection 7442 or part of the central cantilevered projection 7442 may provide a guide or track for the follower 7450. The follower 7450 may include legs 7452 which at least partially fill and displace within the channels 7448 as the follower 7450 is displaced along the central cantilevered projection 7442. As shown, the end region of the central cantilevered projection 7442 opposite the crosspiece 7446 includes a prong 7454. A mounting end of a constant force spring 7456 may be coupled to the prong 7454. The follower 7450 may include a bay which accepts the coil of the constant force spring 7456. A portion of the constant force spring strip extending from the coil to the mounting end may be routed between the follower 7450 and the central cantilevered projection 7442. Though the mounting end is coupled to the central cantilevered projection 7442 via the prong 7454 in the example depicted, any other suitable coupling scheme could be used (e.g. fasteners).

The central cantilevered projection 7442 may include a stop projection 7461 (see, e.g., FIG. 52A) which prevents displacement of the follower 7450 off of the central cantilevered projection 7442 and defines an end of the displacement range for the follower 7450. In alternative examples, there may be a ramp which the follower 7450 may slide over (and perhaps elastically distort around) when the bag vending assembly 7400 is assembled. The ledge face of the ramp may inhibit removal of the follower 7450 once the follower has been advanced over the sloped face of the ramp. Additionally, the follower 7450 may, in some examples, include runner projections 7451 (see, e.g., FIG. 52B) which extend from the legs 7452 toward the opposing leg 7452. The channels 7448 may include wide segments 7449 at ends thereof most proximate the crosspiece 7446. The wide segments 7449 may facilitate installation of the follower 7450. The follower 7450 may be installed on the central cantilevered projection 7442 may passing the legs 7452 and runners 7451 into the wide portion 7449 of the channels 7448. Displacement of the follower toward the opposing end of the central cantilevered projection 7442 may cause the runners 7451 to overhang the underside of the central cantilevered projection 7442 inhibiting removal of the follower 7450 from the bag vending assembly 7400.

Referring now primarily to FIGS. 51B-C, the face of the end region of each lateral cantilevered projection 7444 most proximal the central cantilevered projection 7442 may include a bag retainer 7458. In the example embodiment, the bag retainers 7458 are cantilevered fingers with nubs 7460 that extend toward the central cantilevered projection at their unsupported ends. When the example bag vending assembly 7400 is filled with bags 26, the nubs 7460 and resiliency of the bag retainers 7458 may prevent the lead bag 26 in the bag vending assembly 7400 from being ejected under the urging of the constant force spring 7456. The ports 392 of the lead bag 26 may be grasped by automation equipment (e.g. a bag retainer assembly 4306, see FIG. 68A) and pulled out of the bag vending assembly 7400. The bag retainers 7458 may elastically deflect as the bag 26 is pulled allowing the bag 26 to be freed from the bag vending assembly 7400. Thus, the bag retainers 7458 may act as the vending output for the bag vending assembly 7400. The constant force spring 7456 may drive the follower 7450 in the downstream direction to advance the ports 392 in the downstream direction along the channels 7448 to fill the vacancy created by the removed bag 26.

Though certain example bags 26 are shown in the bag vending assemblies 7400 shown and described in FIGS. 49A-51C, these bags 26 are merely exemplary. Bag vending assemblies 7400 may accept any individual bags 26 such as those shown or described herein. The bags 26 may be provided in an empty state or may be at least partially filled by contents shown or described herein. It may be preferred that the bag 26 include a stopper 38 of the variety depicted in FIG. 6B to increase the capacity or compactness of a bag vending assembly 7400. Flatter butterfly type stoppers 38 may allow bags 26 to be more stored more densely within a bag vending assembly 7400. Bag vending assemblies 7400 may be arranged to store bags 26 with flat, butterfly type stoppers 38. Bag vending assemblies 7400 may be arranged to maximize storage density for bags 26 with butterfly type stoppers 38 while spacing bag retainers 7424 or detents 7430 to allow every other set of bag retainer 7424 or detents 7340 to be populated with a bag 26 having a less space efficient stopper 38 variety (see, e.g., the tulip type stopper 38 shown in FIG. 7A). Additionally, though certain example ports 392 are depicted for the bags 26 is again merely exemplary. Any suitable port 392 may be utilized. Where the ports 392 for a desired bag 26 do not include ribs 140, the ports 392 may be outfitted with at least one attachment or adapter including ribs 140 or other projections. For example, any of the projection bearing bodies 142 shown in FIGS. 10A-B, 16A-18C, the ribbing clip 168 of FIG. 13, the ribbed rings 180 of FIGS. 14A-C, or the ribbed spacers 141 of FIGS. 15A-B may be used.

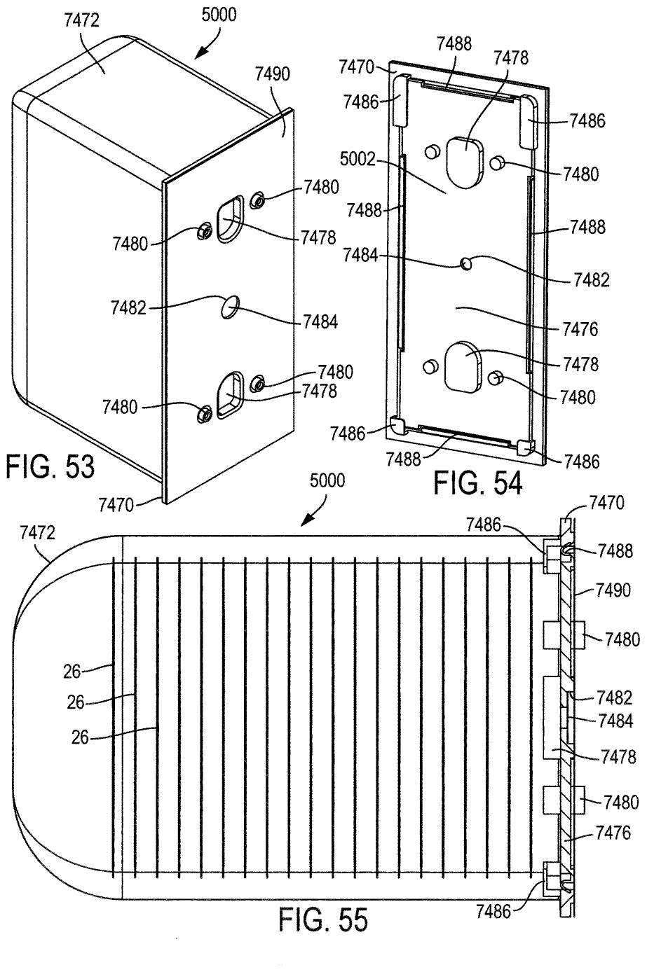

Referring now primarily to FIG. 52A any of the example bag vending assemblies 7400 described and/or shown herein may be fitted into any of the above described dispensers 5000 in place of a spool 2696 of bags 26 in a coupled in a chain 2670. For purposes of example only, a variant of the bag vending assembly 7400 of FIGS. 51A-C is depicted coupled to a second housing portion 5006B of the dispenser 5000 shown in, for example, FIGS. 31-34C. In place of the bag presenter 5004 (see, e.g. FIGS. 38A-B), a bag vending assembly 7400 may be coupled to the interior wall of the second housing portion 5006B adjacent the area where the removable cover assembly 5002 (see, e.g., FIG. 34C) is disposed. In the example, the main body 7440 of the bag vending assembly 7400 includes a peripheral flange 7462 with protrusions or lobes which may accept fasteners for coupling the bag vending assembly 7400 in place. The first portion 7402 of bag vending assemblies 7400 such as those shown and described across FIGS. 49A-50E may include a similar peripheral coupling flange. Bag vending assemblies 7400 may, however, be coupled to the second housing portion 5006B in any suitable manner. Any bag vending assembly 7400 may be disposed such that the lead bag 26 is positioned in line with the outlet 2684 of the dispenser 5000. The ports 392 of bags 26 held by the bag vending assembly 7400 may be aligned in a plane substantially parallel to the front face 5076 (see, e.g. FIG. 38B) of the dispenser 5000. The housing portions 5000A, B could also be modified to have a width closer to the width of the bag vending assembly 7400 to give the dispenser 5000 are narrower footprint as shown in FIG. 52B. The dispenser 5000 may have an aspect ratio closer to that of a bag 26. Though shown coupled to the second housing portion 5006B, a bag vending assembly 7400 may also be similarly coupled to the frame portion 7470 of a dispenser 5000 such as that shown in FIG. 53.

Referring now to FIG. 53-55, certain systems 10 may accept a variety of dispensers 5000. A system 10 may accept dispenser 5000 types with spools 2696 of bags 26 in a chain 26, as well as alternative dispensers 5000 including bag vending assemblies 7400 such as those shown and/or described herein. An example alternative dispenser 5000 well suited for a bag vending assembly 7400 is depicted in FIGS. 53-55. The dispenser 5000 shown in FIGS. 53-55 may include a frame portion 7470 to which a removable cover assembly 5002 is coupled. The frame portion 7470 may include a flange 5008 with a set of pin retainers 5016 similar to those shown in FIG. 33 to interface with a dispenser carriage 5252 of a docking assembly 5250 (see, e.g., FIG. 43). The flange 5008 may interface with, for example, support bodies 5270, 5272 as described in relation to FIG. 43. Alternatively, the frame portion 7470 may also be coupled to a housing portion 5006B with the removable cover portion 5002 positioned in line with an outlet 2684 defined in the housing portion 5006B (see, e.g., FIG. 58A). The flange 5008 on the housing portion 5006B may be utilized for interfacing with the docking assembly 5250 as described elsewhere herein.

The frame portion 7470 may be coupled to a shell 7472 which isolates the bags 26 and bag vending assembly 7400 from the surrounding environment. The shell 7472 may be a tub which is thin and lightweight but resists substantial deformation under typical shipping and handling. Such a tub could for instance be a thermoformed or blow molded component. Alternatively, the shell 7472 may be constructed of a flaccid material which forms an environmental barrier around bag vending assembly 7400 and any bags 26 therein. The shell 7472 could also be formed of a nonwoven spun bond olefin fiber such as a flashspun high-density polyethylene fiber flexible sheeting material. Examples include Tyvek® products available from DuPont. A thin LDPE sheeting material may also be used. In such embodiments, handles may be present on the interior of the shell 7472 (e.g. extending off of a bag vending assembly 7400) and a user may grasp these handles through the flexible shell 7472. Shells 7472 which are partially flaccid and partially formed of a rigid material are also possible (handles would be included preferably in the rigid portion). Where a shell 7472 is at least partially flexible or flaccid the dispenser 5000 may include a rigid skeleton or scaffold (e.g. attached or extending from the frame portion 7470 or a housing portion 5006B which the flexible portion of shell 7472 may drape over. This may assist in keeping shell 7472 material clear of bags 26 or other components within the dispenser 5000.

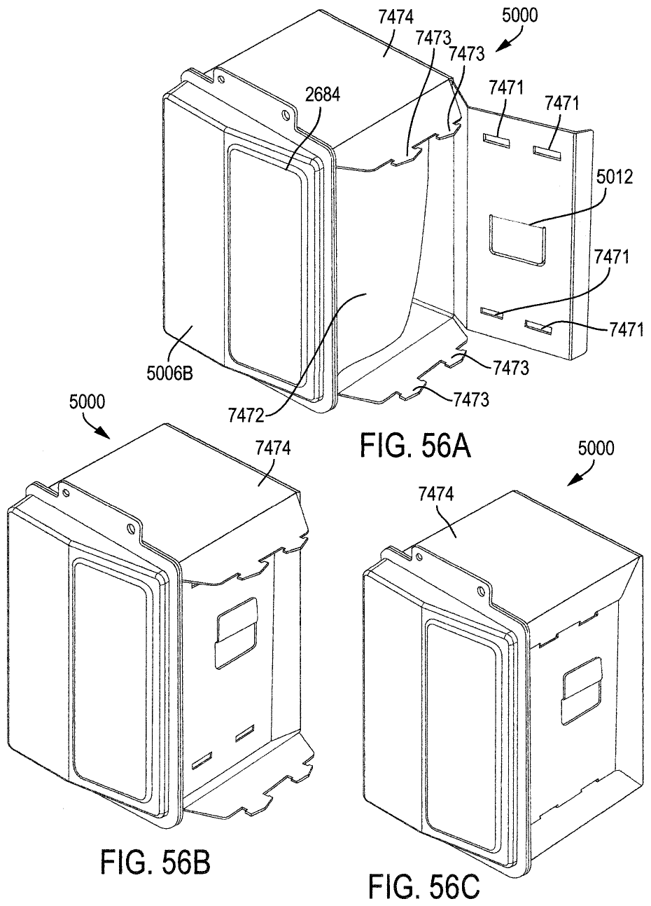

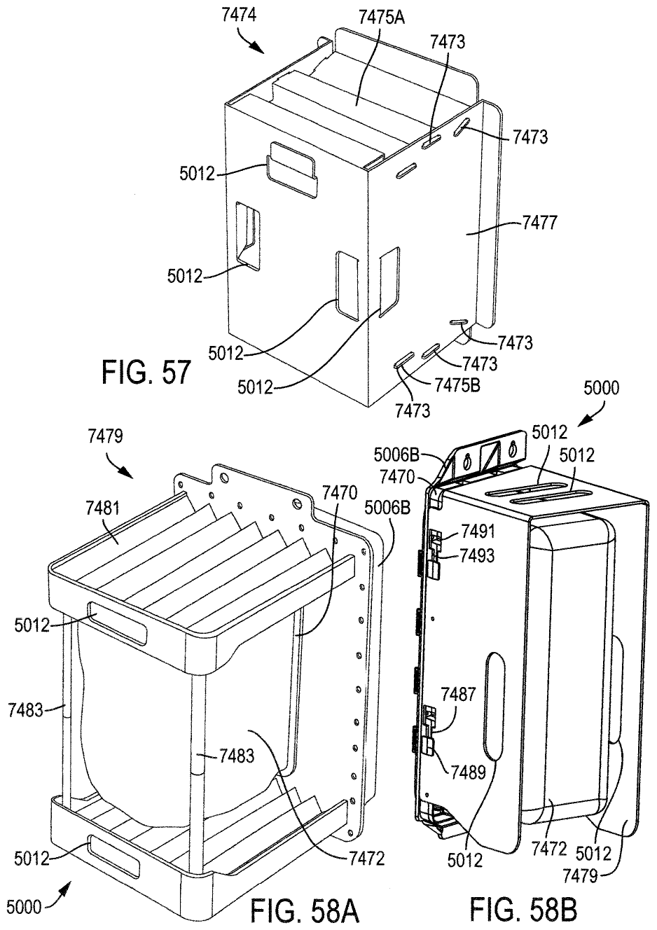

Referring now also to FIGS. 56A-57, in certain examples, the dispenser 5000 may include an outer housing 7474. In the example shown, the outer housing 7474 is coupled to a housing portion 5006B within which a shell 7472 is contained. This is merely exemplary, an outer housing 7474 may also be coupled to a frame portion 7470. The outer housing 7474 may have a rigidity greater than the shell 7472. The outer housing 7474 may provide no environmental seal (though may play a role in assisting in blocking ingress of particles and other detritus). The shell 7472 may provide an environmental seal. Where the shell 7472 material is flaccid, for example, the outer housing 7474 may block contact with the shell 7472 during handling to inhibit inadvertent application of force to the bags 26 within the dispenser 5000. The outer housing 7474 may be constructed of a material such as a corrugated plastic (e.g. polypropylene other suitable polymer) plate sheet material. This may assist in providing a lightweight dispenser 5000 which may be easily handled.

Where such material is used, the outer housing 7474 may be a folding carton type construction. For example, the material may include panels with tabs, flaps, or other closure projections 7473 which may be folded and/or tucked into slits 7471 of other panels to hold the outer housing 7474 in an intended shape. Handles 5012 may be defined in the outer housing 7474 to facilitate grasping of the dispenser 5000 by a user. FIG. 56A depicts an example dispenser 5000 where the outer housing 7474 is partially assembled. The flaps and tabs 7473 may be folded and interlocked into place with other panels to complete assembly of the outer housing 7474 (see, e.g., FIG. 56B-C). Another example outer housing 7474 is depicted in FIG. 57. End panels 7475A, B may be coupled to the top and bottom of the frame 7470 or a housing portion 5006B (neither shown) and may have tabs 7473 about their peripheries. A sidewall portion 7477 of the outer housing 7474 may be folded around the peripheries of the end panels 7475A, B and retained in place via the tabs 7473. The end panels 7475A, B may be folded in a peak and trough pattern to provide additional rigidity.

In still other examples, and referring now to FIG. 58A, a partial outer housing 7479 may be included. The partial outer housing 7479 may act as a bumper or cage around the shell 7472 which assists in grasping of the dispenser 5000 and limits opportunities for the shell 7472 to be contacted during shipping or handling. The partial outer shell 7479 may include end cap portions 7481 which include handles 5012. A number of struts 7483 may end between the end cap portions 7481. The struts 7483 may also be grasped and serve as handles for the dispenser 5000. The shell 7472 may be open to the surrounding environment but the end cap portions 7481 and struts 7483 may help block, for example, impacts from other dispensers 5000 during shipping or manipulation during inventory management. Again, this may assist in providing a lightweight and easy to maneuver dispenser 5000.

Referring now to FIG. 58B, another example partial outer housing 7479 is depicted. The partial outer housing 7479 may be formed of corrugated plastic and may surround three sides of the shell 7472. The partial outer housing 7479 may include a number of handles 5012 which may be cut or punched out of the material forming the partial outer housing 7479. As shown, a housing portion 5006B or the periphery of the frame portion 7470 of a dispenser 5000 may include projections 7487 with enlarged head regions 7489. The partial outer housing 7479 (and other outer housings 7474) may include passages 7491 with thin width tail extension 7493. The head regions 7489 may be passed through the passage 7491 and the partial outer housing 7479 may be displaced to side the projections 7487 into the tail extensions 7493. This may couple the partial outer housing 7479 to the dispenser 5000 as head regions 7489 may be too wide to pass through the tail extensions 7493. Typically, the tail extensions 7493 may be oriented such that the weight of the dispenser will tend to maintain the projections 7487 in the tail extensions 7493 as the dispenser 5000 is handled.

Referring again primarily to FIGS. 53-55, the removable cover assembly 5002 may include a panel body 7476. The panel body 7476 may be substantially planar but may include a set of depressions 7478 spaced along the centerline of the panel body 7476. The panel body 7476 may also include a number of guide recesses 7480. In the example embodiment, a pair of guide recesses 7480 flank either side of the depressions 7478. The panel body 7476 additionally includes a port 7482 which is spanned by a sterile barrier 7484 which permits passage of air/vapor. The barrier 7484 may, for example, be formed of a nonwoven spun bond olefin fiber such as a flashspun high-density polyethylene fiber flexible sheeting material. Examples include Tyvek®_products available from DuPont. As best shown in FIG. 54, the frame 7470 may include retainers 7486 which assist in holding the removable cover assembly 5002 in the dispenser 5000 and prevent excessive displacement of the removable cover assembly 5002 toward the interior of the dispenser 5000. At least two opposing edges of the panel body 7476 may include spring clips 7488 along at least a portion of those edges. In the example embodiment, the spring clips 7488 are formed monolithically with the panel body 7476 (e.g. during injection molding) but could be coupled to the panel body 7476 in alterative examples.

Still referring to primarily to FIGS. 53-55, the dispenser 5000 may include a frangible 7490. The frangible 7490 may be provided in the form of a sheet of flexible material or membrane material which is coupled over the exterior surface of the frame 7470 and removable cover assembly 5002. The frangible 7490 may include apertures which align with the depressions 7478, guide recesses 7480, and port 7482 of the panel body 7476. The frangible 7490 may be coupled to the frame 7470 and the panel body 7476 in any suitable manner (e.g. heat stake, sonic welding, laser welding, solvent bonding, adhesive, etc.). In certain examples, a polymer film (e.g. polyethylene terephthalate) may be used for the frangible 7490. The frangible 7490 may form an environmental barrier for the dispenser 5000 which maintains the interior volume of the dispenser 5000 in a sealed state after terminal sterilization of the dispenser 5000.

Though the example frangible 7490 covered frame portion 7470 and removable cover assembly 5002 are described in relation to the embodiment shown in FIGS. 53-55, it should be understood that such an arrangement may be included on any dispenser 5000 shown or described herein. For example, the overlay body 5030, lock bodies 5032A, B, backing body 5034, rim 5054, bolt retainers 5052 (see, e.g., FIG. 34C for each of the foregoing), and other components associated with the removable cover assembly 5002 shown on the dispenser 5000 of FIGS. 31-41B may be omitted. These components may be replaced with the example frangible 7490 covered frame portion 7470 and removable cover assembly 5002 depicted in FIGS. 53-55.

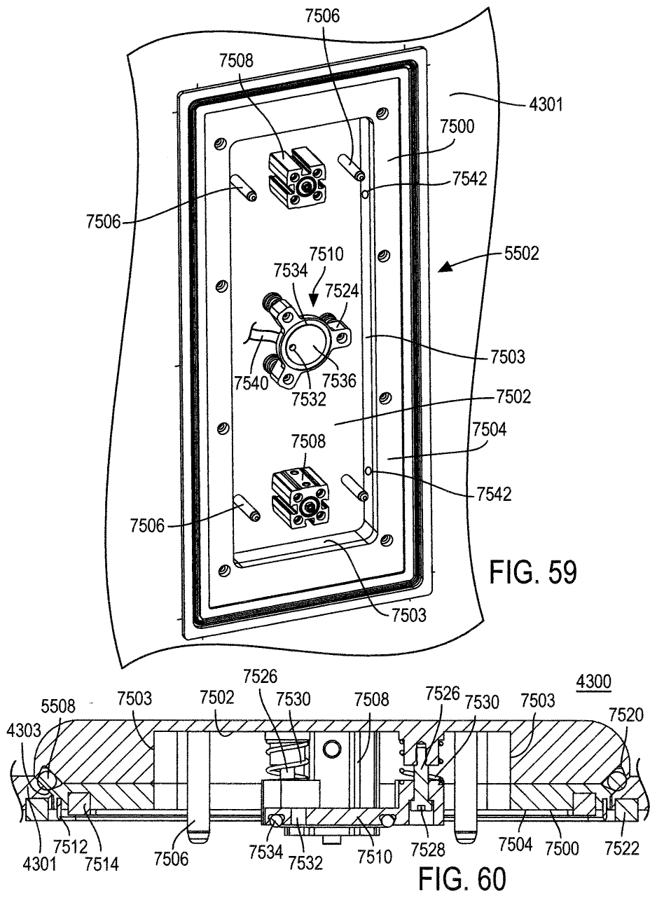

Referring now to FIGS. 59-60, a perspective and cross-sectional view of an example infeed aperture plug 5502 in a plugging state against an infeed aperture 4303 of a processing compartment 4300 are respectively depicted. The infeed aperture plug 5502 may be included in any system 10 which interfaces with a dispenser 5000 including a frangible 7490 covered frame portion 7470 and removable cover assembly 5002 such as that shown and described in relation to FIGS. 53-55. The infeed aperture plug 5502 may, for example, replace that shown in FIG. 43 and FIG. 45. The example infeed aperture plug 5502 described in relation to FIGS. 59-60, may, however, be coupled to plug displacement actuators 5522, 5524 (see, e.g., FIG. 42) and displace between a plugging and stowed position as described elsewhere herein.

As shown, the infeed aperture plug 5502 may include an exposed side 7500 including a recessed central region 7502 and a surrounding raised plateau face 7504. The recessed central region 7502 may include sidewalls 7503 extending from the raised plateau face 7504 to the deepest portion of the recessed central region 7502. Within the recessed central region 7502 may be a set of guidance pins 7506. The guidance pins 7506 may be tapered at their unsupported ends. The guidance pins 7506 may be positioned to generally align with respective guide recesses 7480 of the removable cover assembly 5002 of a dispenser 5000. The guidance pins 7506 may help to place the dispenser 5000 in a prescribed positional relationship relative to the infeed aperture plug 5502 as a docking assembly advances the dispenser 5000 in the direction of the infeed aperture plug 5502. A set of linear actuators 7508 may also be disposed in the recessed central region 7502 and may be spaced along the midline of the infeed aperture plug 5502. The linear actuators 7508 may align with respective depression 7478 of the removable cover assembly 5002. A port sealing assembly 7510 is additionally disposed within the recessed central region 7502 and may form a seal against the frangible 7490 around the port 7482 when the dispenser 5000 is advanced a certain amount toward the fully installed position by a docking assembly 5250 of a system 10.

Adjacent the periphery of the surrounding raised plateau face 7504, a cutter 7512 may be disposed. The cutter 7512 may extend entirely around the recessed central region 7502. The cutter 7512 may, in some examples, be a steel (or other metallic material) rule die. The blade of the cutter 7512 may preferably be serrated. Inboard of, but adjacent to the cutter 7512 a sealing member 7514 may be coupled to the surrounding raised plateau face 7504. The sealing member 7514 may similarly surround the entire recessed central region 7502.

The infeed aperture 4303 in the processing chamber wall 4301 may include a chamfered face which increases the cross-sectional area of the aperture 4303 as distance to the interior of the processing chamber 4300 decreases. A second portion 7516 of the infeed aperture plug 5502 may include a perimeter sealing member 5508 which may compress against the chamfered face when in a plugging state. This may form a seal between the interior of the processing compartment 4300 and the docking compartment 5258 of the docking assembly 5250 (see, e.g., FIG. 42). The surrounding raised plateau face 7504 of the exposed portion 7500 of the infeed aperture plug 5502 may be included as a face plate which is coupled to the second portion 7516. This may facilitate creation of a dovetail type groove 7520 geometry for retention of the perimeter sealing member 5508 as a portion of the rear side of the face plate may form a portion of such a groove 7520. The exterior face of the processing chamber wall 4301 may also include a sealing member 7522 which may surround the infeed aperture 4303.

Still referring to FIGS. 59-60, the port sealing assembly 7510 may include a carriage body 7524 which may displace toward and away from the surface of the recessed central portion 7502 of the infeed aperture plug 5502 along guide bodies 7526. In the example, the guide bodies 7526 are shoulder bolts which extend through passages in the carriage body 7524 and thread into receptacles in the recessed central portion 7502. The carriage body 7524 is displaceable along the unthreaded portion of the guide bodies 7526 with the displacement range of the carriage body 7524 being limited by the heads 7528 of the guide bodies 7526. The carriage body 7524 may be biased toward a first end of its displacement range against these heads 7528 by one or more bias member 7530. The carriage body 7524 includes a passage 7532 extending through the carriage body 7524 to a portion of an exterior face 7536 of the carriage body 7524 which is surrounded by a carriage sealing member 7534.

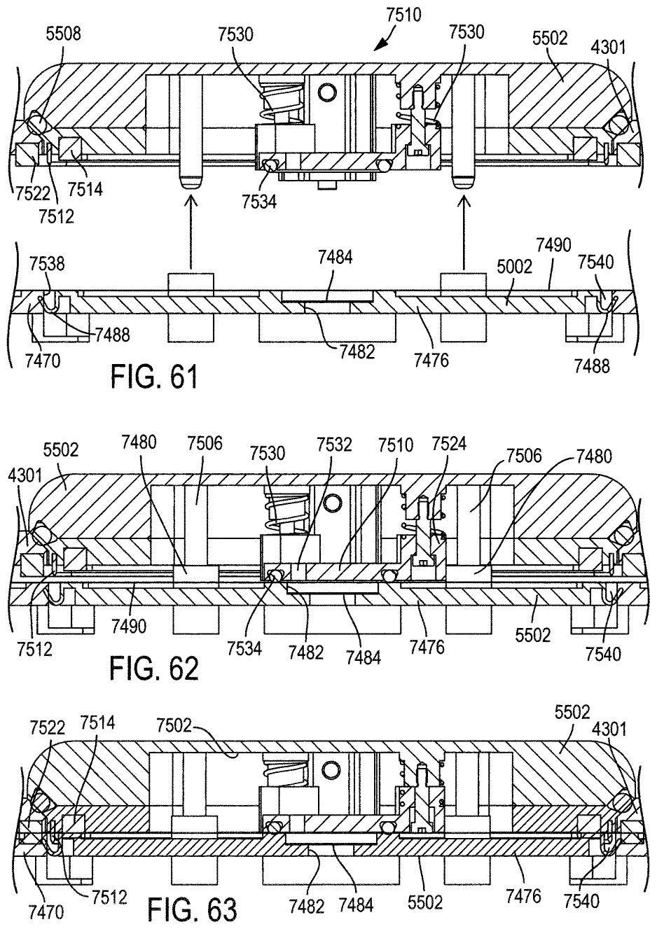

Referring now to the progression of FIGS. 61-63, a dispenser 5000 is shown being displaced through a partially installed position and into a fully installed position against the infeed aperture plug 5502 and processing compartment wall 4301. The shell 7472 (see, e.g., FIG. 53) is not depicted in FIGS. 61-63 for simplicity of illustration. As shown in FIG. 61, the dispenser 5000 may be advanced toward the infeed aperture plug 5502 (by e.g. a docking assembly 5250 as described elsewhere herein) with the frangible 7490 in an uninterrupted state. The spring clips 7488 may press against sidewalls 7538 of the frame portion 7470 holding the removable cover assembly 5502 in place. A small gap or crevice 7540 may be present between the frame portion 7470 and the portion of the panel body 7476 from which the spring clips 7488 extend.

As the dispenser 5000 is brought to a partially installed position (see, e.g., FIG. 62), the guide recesses 7480 of the removable cover assembly 5002 may advance onto the guidance pines 7506. This may ensure that the dispenser 5000 is located in a prescribed relative position to the infeed aperture plug 5502 as the dispenser 5000 is further displaced in the installation direction. As installation proceeds, the frangible 7490 may contact the port sealing assembly 7510 (see, e.g., FIG. 62) and the carriage sealing member 7534 may transition to a compressed state as it is pressed against the panel body 7476 with the frangible 7490 being sandwiched between the two. This may form a seal around the port 7482 of the removable cover assembly 5002. The bias member(s) 7530 of the port sealing assembly 7510 may assist in forming a seal against the frangible 7490 with any displacement of the carriage body 7524 toward the recessed central portion 7502 (see, e.g., FIG. 63). As shown best in FIG. 62, a seal between the carriage sealing member 7536 and the frangible 7490 may be established prior to the cutter 7512 contacting the frangible 7490.

The passage 7532 may be in selective fluid communication with a pressure supply via a run of tubing 7540 (see, e.g., FIG. 59) and one or more valve (not shown). Thus, the passage 7532 may be referred to herein as a pressure port. A pressure sensor 5520 may also be or may be selectively placed (e.g. via a valve) in communication with the passage 7532. Pressure (preferably positive) may be supplied through the passage 7532 and barrier 7484 to the interior volume of the dispenser 5000. The control system 15 may monitor the pressure indicated by the output signal of the pressure sensor 5520 and may isolate the pressure supply from the interior volume of the dispenser 5000 when a predetermined test pressure is reached. The control system 15 may continue to monitor the output signal for a preset period of time. In the event that the pressure decays by more than a predefined threshold amount or the derivative of the pressure exceeds a predefined threshold, the control system 15 may inhibit use of the dispenser 5000. In the event pressure decay characteristics observed by monitoring the output of the pressure sensor 5520 are acceptable, the control system 15 may continue orchestrating installation of the dispenser 5000. Preferably, the control system 15 may also return the interior volume of the dispenser 5000 into communication with the pressure supply to maintain a positive pressure in the dispenser 5000.

Though described in relation to FIG. 62, dispenser 5000 integrity testing may be performed with any dispenser 5000 shown or described herein. It shall be understood that other dispenser 5000 varieties shown and described herein may be modified to include a port 7482 and barrier 7484. Likewise, other infeed aperture plugs 5502 may be modified to include a port sealing assembly 7510. Other dispensers 5000 may similarly be maintained at a positive pressure during at least part of the installation by means of these components as well.

Additionally, though dispensers 5000 are described in relation to supply of reservoirs to a system 10, it shall be appreciated that dispensers 5000 of the types shown and described herein may supply other components, materials, agents, etc. to controlled environments other than that exemplary ones shown and described herein. Any controlled environment may incorporate an infeed aperture plug 5502 such as those shown and described herein which interfaces with dispensers 5000 such as those shown and described herein. In certain examples, it may be desirable to supply negative pressure through the pressure port 7532. Where a dispenser 5000 may include, for example, cytotoxic, caustic, or other or hazardous materials, negative pressure may be utilized to evacuate the dispenser 5000 before it is accessed. Evacuated gas from the dispenser 5000 may be sampled or tested before the dispenser 5000 is accessed.

Referring now primarily to FIG. 63, the dispenser 5000 is shown in a fully installed position against the infeed aperture plug 5502. In the fully installed position, the frangible 7490 portion overlaying the frame portion 7470 may be compressed against the sealing member 7522 surrounding the infeed aperture 4303. The portion of the frangible 7490 overlaying the periphery of the removable cover assembly 5002 may be compressed against the sealing member 7514 surrounding the recessed central region 7502 of the infeed aperture plug 5502. The cutter 7512 may compromise the frangible 7490 as the dispenser is displaced to the fully installed position. As shown, the crevice 7538 between the frame portion 7470 and panel body 7476 may accept the blade portion of the cutter 7512 in the fully installed position. The carriage 7524 of the test port assembly 7510 may also be displaced toward the second end of its displacement range compressing any bias member(s) 7530 included in the test port assembly 7510.

Still referring to primarily to FIG. 63, the seal formed between the frame portion 7470 and the sealing member 7522 surrounding the infeed aperture 4303 may provide an environmental seal which isolates the infeed aperture 4303 from the ambient environment. The seal formed between the perimeter of the removable cover assembly 5502 and the sealing member 7514 surrounding the recessed central region 7512 may be used to permit application of a retaining vacuum to the volume intermediate the recessed central region 7502 and the removable cover assembly 5002. The retaining vacuum may be sufficient to firmly retain the removable cover assembly 5002 against the infeed aperture plug 5502. One or more vacuum port 7542 (see, e.g., FIG. 59) associated with the recessed central region 7502 may be placed into communication with a vacuum source 5517 (see, e.g., FIG. 42) via a valve 5518 (see, e.g., FIG. 42). In some examples, negative pressure may be supplied to the recessed central region 7502 prior to the dispenser 5000 being brought to the fully installed position. The bias member(s) 7530 of the test port assembly 7510 may be selected to overcome the force from the differential pressure between the interior volume of the dispenser 5000 and the retaining vacuum applied to the volume between the recessed central region 7502 and removable cover assembly 5002 (without over powering the retaining vacuum). Preferably, a seal between the dispenser 5000 and the sealing member 7522 surrounding the infeed aperture 4303 is formed prior to the cutter 7512 breaching the frangible 7490. Alternatively, the interior volume of the dispenser 5000 may be kept at a positive pressure as the dispenser 5000 is displaced to a fully installed position. Thus, in the event the frangible 7490 is breached prior to a seal between the sealing member 7522 and frame portion 7470 forming, positive pressure in the interior volume of the dispenser 5000 may ensure no ambient air enters the dispenser 5000.

With the dispenser 5000 at the fully installed position and the vacuum source 5517 in communication with the volume intermediate the recessed central region 7502 and removable cover assembly 5002, at least one second integrity test may be conducted. A data signal from a pressure sensor 5520 (see, e.g., FIG. 42) in communication with the volume intermediate the recessed central region 7502 and removable cover assembly 5002 may be monitored to ensure that vacuum established in this intermediate volume meets one or more predefined criteria. For example, the communication to the pressure source 5517 may be interrupted and the data signal from the pressure sensor 5520 may be monitored by the control system 15 to check that decay characteristics (e.g. total pressure decay, derivative of pressure) are in accordance with predefined thresholds. The control system 15 may also monitor the decay or decay rate of any positive pressure in the interior volume of the dispenser 5000 is in conformity with predefined thresholds. A pressure sensor 5520 may be placed into communication with this volume through the test port 7480 and the control system 15 may monitor the data signal produced by this sensor. In some embodiments, the system 10 may instead or additionally include flow sensor monitoring flow of air to the interior of the dispenser 5000 and from the intermediary volume between the removable cover assembly 5002 and recessed central region 7502. The control system 15 may monitor the data signal from any such flow sensors and ensure the flow or flow rate is in conformance with predetermined thresholds. The control system 15 may prohibit removal of the removable cover assembly 5002 from the dispenser 5000 in the event any integrity criteria are violated.

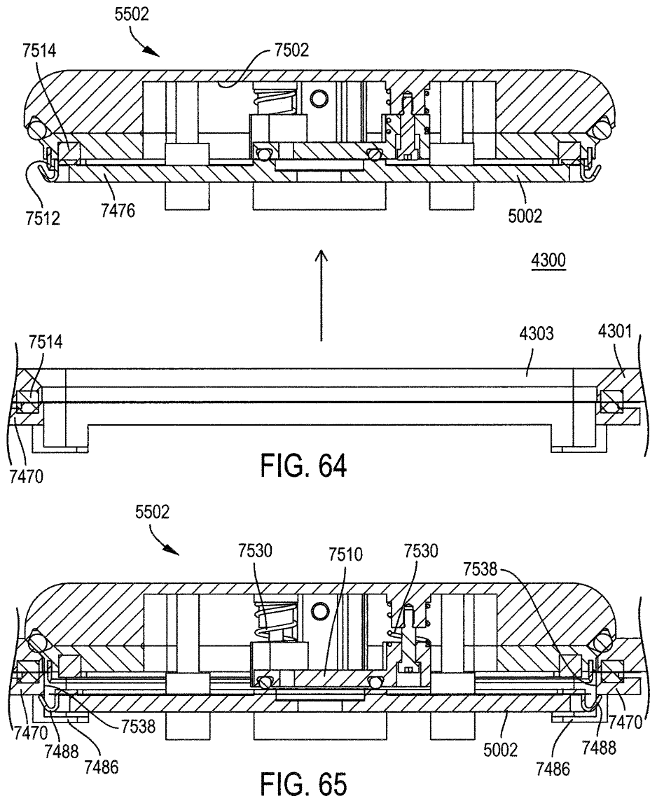

Referring now to FIG. 64, the infeed aperture plug 5502 is shown displaced toward a stowed position with the removable cover assembly 5002 of the dispenser 5000 retained on the infeed aperture plug 5502. As mentioned above, this retention may be accomplished by maintaining a retaining vacuum between the removable cover assembly 5002 and the infeed aperture plug 5502. The stowed position may, for example, be the position shown in FIG. 2. The infeed aperture plug 5502 may be displaced via plug displacement actuators 5522, 5524 (see, e.g., FIG. 42) as described in relation to FIG. 42. The seal formed due to compression of the sealing member 7514 surrounding the recessed central region 7502 against the removable cover assembly 5502 may keep the previously exposed areas of the removable cover assembly 5502 and infeed aperture plug 5502 segregated from the interior of the processing compartment 4300.

The frame portion 7470 is shown in place against the sealing member 7522 on the exterior of the processing compartment wall 4301. The shell 7472 (see, e.g., FIG. 53) is not depicted in FIG. 65 for simplicity of illustration. Compression of the sealing member 7522 surrounding the infeed aperture 4303 against the frame portion 7470 may maintain environmental isolation of both the processing compartment 4300 and interior volume of the dispenser 5000 from the surrounding ambient environment. The portion of the frangible 7490 coupled to the frame portion 7470 may be sandwiched between the sealing member 7522 and the frame portion 7470. The portion of the frangible 7490 cut out by the cutter 7512 may be carried to the stowed position with the infeed aperture plug 5502. Positive pressure supply to the test port assembly 7510 may optionally be halted. With the infeed aperture plug 5502 and removable cover assembly 5002 displaced to the stowed position, a bag displacement assembly 2618 may collect bags 26 from the dispenser 5000 for downstream filling/processing.

Once the dispenser 5000 is depleted, the infeed aperture plug 5502 and removable cover assembly 5002 may be displaced back to the infeed aperture 4303. The infeed aperture plug 5502 and cover assembly 5002 would be in the positions shown in FIG. 63. The retaining vacuum applied to the intermediary volume between the removable cover assembly 5002 and the infeed aperture plug 5502 may be terminated. The vacuum ports 7542 (see, e.g., FIG. 59) may be placed into communication with a vent (e.g. to ambient) via actuation of one or more valves to assist in terminating the vacuum. As shown in FIG. 65, this may result in displacement of the removable cover assembly 5002 toward the interior volume of the dispenser 5000 (the shell 7472 is not depicted in FIG. 65 for ease of illustration). This displacement may occur as any bias member(s) 7530 of the port sealing assembly 7510 relax to a less stressed state once the retaining vacuum has abated. In some examples, the bias exerted by the bias member(s) 7530 of the port sealing assembly 7510 may be sufficient to ensure that the spring clips 7488 of the removable cover assembly 5002 are driven off the sidewalls 7538 of the frame portion 7470 toward the interior volume of the dispenser 5000. The spring clips 7488 may be in a deflected state when in alignment with the sidewalls 7538 and may restore to an unstressed state when displaced off the sidewall 7538. The spring clips 7488 may then catch on a face of the frame portion 7470 most proximal the interior volume of the dispenser 5000 presenting a mechanical interference to removal of the removable cover assembly 5002 from the dispenser 5000. The retainers 7486 of the frame portion 7470 may prevent the removable cover assembly 5002 from becoming loose within the dispenser 5000.

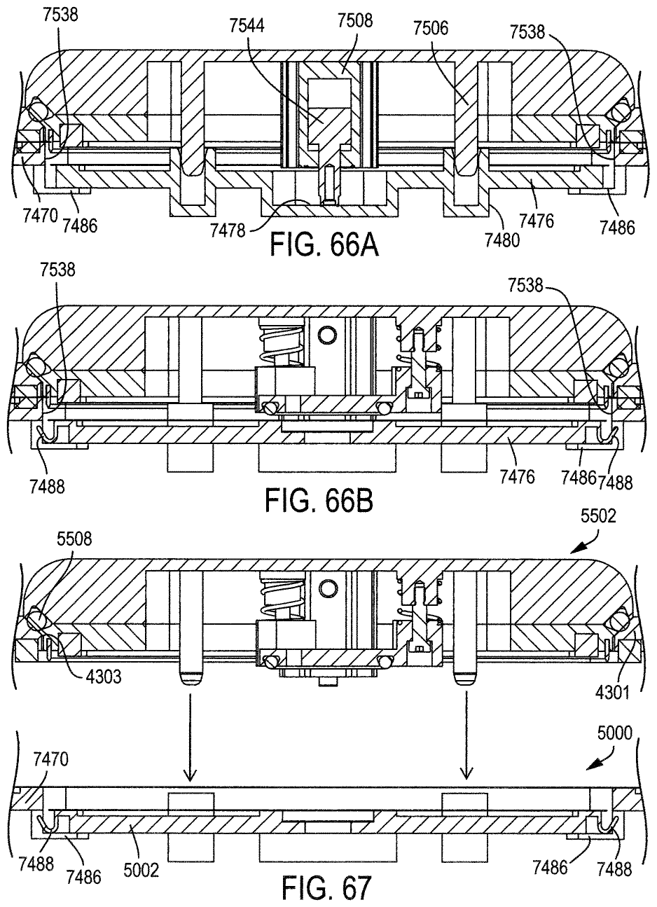

Optionally and referring now to FIGS. 66A-B, in certain examples, the infeed aperture plug 5502 may optionally include a set of linear actuators 7508 (best shown in FIG. 66A). The linear actuators 7508 may each include a pneumatically driven plunger 7544. Pressure may be supplied to the linear actuators 7508 to drive the plungers 7544 in an ejection direction and against the depressions 7478 of the panel body 7476. This may force the removable cover assembly 5002 to displace such that the spring clips 7488 are driven off the sidewalls 7538 of the frame portion 7470 as shown best in FIG. 66B. The retainers 7486 of the frame portion 7470 may ensure that displacement of the removable cover assembly 5002 into the interior of the dispenser 5000 (shell 7472 not shown for simplicity of illustration) is limited as the plungers 7544 are extended to an ejecting state. Each linear actuator 7508 may include a plunger position sensor (e.g. a magnetic sensor monitoring a magnetic body on the plunger 7544) which is monitored by the control system 15. The control system 15 may confirm the plungers 7544 deploy to the ejection state and may generate a notification for display on the user interface 6100 and halt further operation in the event this is not detected. As shown, the guidance pins 7506 and guide recesses 7480 may maintain the removable cover assembly 5002 in a prescribed orientation relative to the infeed aperture plug 5502 as the removable cover assembly 5002 is ejected.

Referring now to FIG. 67, with the removable cover assembly 5002 retained behind the frame portion 7470 by the spring clips 7488 the dispenser 5000 may be undocked from the infeed aperture plug 5502. The dispenser 5000 is shown with the shell 7472 hidden in FIG. 67 for ease of illustration. Carriage actuators 5256 (see, e.g., FIG. 43) of docking assembly 5250 (see, e.g., FIG. 43) may power dispenser carriages 5252 (see, e.g., FIG. 43) away from the processing compartment 4300. The docking compartment 5258 may then be made user accessible (as described in relation to FIG. 42 for example) and the depleted dispenser 5000 may be removed and replaced. The infeed aperture plug 5502 may maintain a seal against the infeed aperture 4303 via sealing member 5508 which keeps the processing compartment 4300 isolated from the environment in the docking compartment 5258.

Referring now to FIG. 68A, a top plan view of a portion of a system 10 is depicted. The top down view includes an example bag displacement assembly 2618 disposed in a processing compartment 4300 of the enclosure 12. Various components for the system 10 have been hidden for sake of illustration. The example bag displacement assembly 2618 may be arranged to be particularly compact as well as hygienic. This may help to ensure that the processing compartment 4300 has minimal sources of particulate and can be kept to a relatively constrained footprint.

Referring now also to FIG. 68B, the bag displacement assembly 2618 may include an upstream displacement assembly 4302 and a downstream displacement assembly 4304. Each includes a bag retainer assembly 4306 which in the example embodiment includes pneumatic graspers. The exemplary bag retainer assemblies 4306 each include a set of jaws 4308A, B. At least one of the jaws 4308A, B of each set is actuatable (e.g. pneumatically) toward the other. The jaws 4308A, B may include recesses 4310 which may accept and close around ports 392 of the bags 26 in order to retain a bag 26 in place. The ports 392 of each bag 26 may be structured to facilitate interface with the jaws 4308A, B as described in greater detail elsewhere herein (see, e.g., FIGS. 10A-21F). The bag retainer assemblies 4306 may be coupled to respective displacement stages or carriages 4318A, B. The bag retainer assembly 4306 of the downstream displacement assembly 4304 is disposed on the unsupported end of an arm 4348 extending from the displacement stage 4318B of the downstream displacement assembly 4304. The displacement stages 4318A, B may displace along dedicated linear guides 4316A, B under power of respective motors 4320 (see, e.g., FIG. 2).

Motion may be communicated from the motors 4320 to the carriages 4318A, B via any suitable transmission, though ball screws may be preferred. The motors 4320 are positioned outside of the processing chamber 4300 to help facilitate maintenance of the controlled environment(s) within the processing chamber 4300 in the example. Alternatively, cleanroom rated motors 4320 could be utilized and positioned inside the processing chamber 4300. The position of the displacement stages 4318A, B may be tracked via a signal output from a rotary encoder monitoring the motor 4320 included in the motor assembly. Any other suitable position sensor may be utilized in alternative examples.

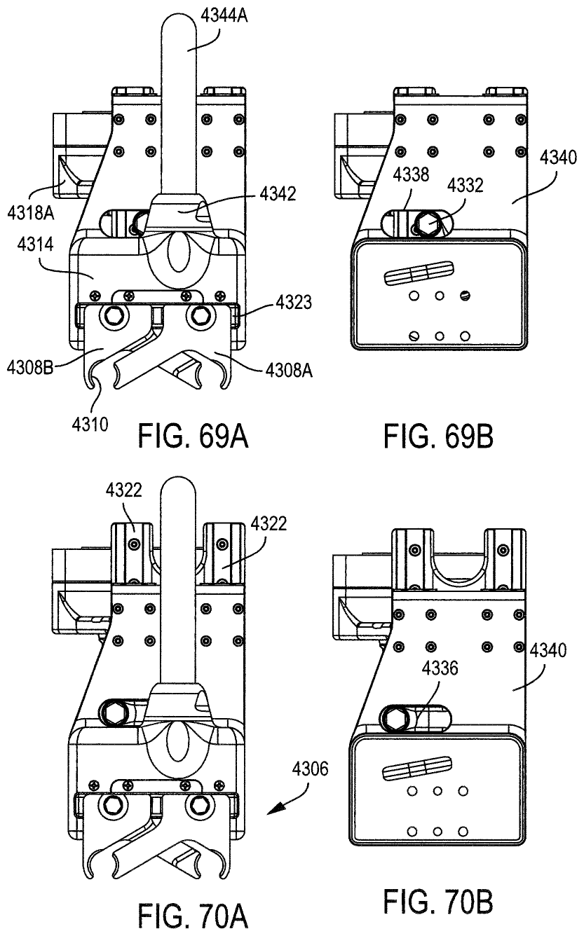

Referring now also to FIGS. 69A-72A, the bag retainers 4306 may also be translationally displaceable relative to their respective displacement stage 4318A, B. A bag retainer housing 4314 is included in each of the example bag retainer assemblies 4206. Each bag retainer housing 4314 may include bearings 4324 which may displace along guides 4322 that are fixed relative to their parent displacement stage 4318A, B. Thus, the bag retainers 4306 may be extended from and retracted towards their parent displacement stage 4318A, B. The extension actuator assembly 4330 for each bag retainer 4306 may be enclosed within an actuator housing 4328. The actuator housing 4328 may be defined by a portion of the respective displacement stage 4318A, B and a cover 4362. One or more sealing member may be included at the interface between the cover 4362 and the displacement stage 4318A, B. Each jaw 4308A, B may include a bearing 4325 which may displace along a jaw bearing guide 4323 defined on the retainer housing 4314.

In certain examples, each extension actuation assembly 4330 includes a cam follower 4332 on a crank 4336 which is displaced about a rotation axis by a rotary actuator 4334 (see, e.g., FIG. 72A). The rotary actuator 4334 may preferably include a harmonic gear motor in various examples. The cam follower 4332 may be disposed within an elongate slot 4338 defined in a base body 4340 of the bag retainer assembly 4306. As shown best in FIG. 69B and FIG. 70B, as the crank 4336 is rotationally displaced, the cam follower 4332 may engender translational displacement of the bag retainer assembly 4306. Rotation of the crank 4336 in a first direction may displace the bag retainer assembly 4306 distal to the displacement stage 4318A, B. Rotation in a second, opposite direction retracts the bag retainer assembly 4306 toward the displacement stage 4318A, B. The displacement range of the bag retainer assembly 4306 relative to the respective displacement stage 4318A, B may be dictated by the elongated slot 4338 and the dimensions of the crank 4338. In the example embodiment, the elongate slot 4338 is sized to permit a 180° rotation of the crank 4338 (the crank 4336 could be rotated another 90° from in position in FIG. 70B). In some embodiments, the crank 4338 may be rotated in a single direction. A first 180° of each full rotation would displace the bag retainer assembly 4306 in a first translational direction and the other 180° of rotation would engender translational displacement of the bag retainer assembly 4306 in the opposing direction. The rotary actuator 4334 may include a rotary encoder (e.g. absolute rotary encoder) which may output a signal indicative of the position of the bag retainer assembly 4306 relative to the parent displacement stage 4318A, B. This signal may be monitored by the control system 15 to track the position of the bag retainer assembly 4306 and inform commands supplied to the rotary actuator 4334. In other embodiments, any other suitable position sensor may be used.

At least one bag sensor 4312A, B may be included in a housing 4314 for the bag retainers 4306. Each of the at least one sensor 4312A, B may be disposed so as to detect the presence of a port 392. The bag sensors 4312A, B may be any suitable variety of sensor. In some examples, reflectivity based sensors may be used. Reflective fiber optic based sensors may be used in certain implementations. Other embodiments could include, for example, microswitches which are actuated when the ports 392 are present. The output signal from any bag sensors 4312A, B may be checked by the control system 15 before a command to actuate the bag retainer 4306 of a respective displacement assembly 4202, 4304 is issued. The bag retainer 4306 may not be actuated in the event that the output signal from one or more bag sensor 4312A, B indicates a bag 26 is absent or improperly positioned. The control system 15 may also monitor the bag sensors 4312A, B for a change in sensor output signals indicating a bag 26 has become dislodged. Any other passive or active holders for ports 392 (and/or priming lines 3120) described herein may include such sensors and be similarly monitored.

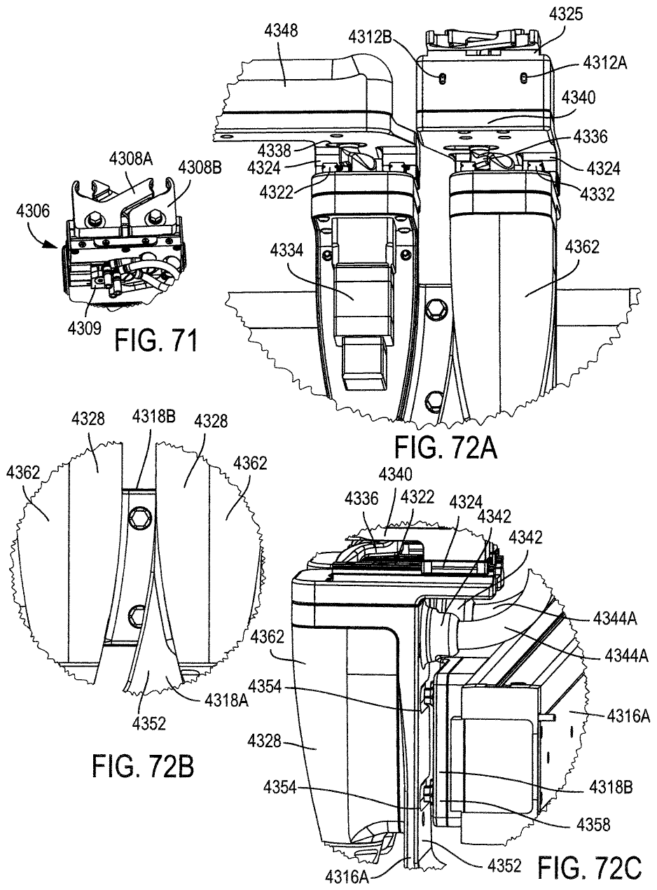

As shown in FIG. 71, the position of the jaws 4038A, B may also be monitored by one or more jaw position sensor 4309. In the example, the jaw position sensor 4309 may be disposed within the bag retainer housing 4314 (hidden in FIG. 71). The jaws 4308A, B may be displaced via a pneumatic cylinder. Jaw position sensors 4309 may be Hall effect type sensors monitoring a magnetic body coupled to the pneumatic cylinder piston in some implementations. Any other suitable sensor variety may be used. Such jaw position sensors 4309 may be included in any of the active gripping assemblies described herein.

The output from the jaw position sensor(s) 4309 may be used to confirm a bag 26 is retained by a bag retainer 4306 (or other active gripper). For example, the jaws 4308A, B may have a closed position and an open position. When commanded closed upon a port 392, the jaws 4308A, B may be blocked from reaching the closed position by the port 392. When a bag 26 is expected to be held by a bag retainer 4306, the control system 15 may verify that the output signal of the jaw position sensor(s) 4309 indicate the jaws 4308A, B are in a partially closed position indicating the bag 26 ports 392 are present in the jaws 4308A, B. The control system 15 may also verify that the output signal of the jaw position sensor(s) 4309 is/are indicative of the jaws 4308A, B being in an open position after commanding a bag 26 be released by a bag retainer assembly 4306. In the event that an unexpected jaw 4308 position is detected, the control system 15 may inhibit displacement of the bag retainer assembly 4306. A notification may also be generated by the control system 15 (e.g. on the user interface 6100). The control system 15 may also monitor the jaw position sensor(s) 4309 for a change in sensor output signals indicating a bag 26 retained in the jaws 4308A, B has become dislodged. Any other active holders for ports 392 (and/or priming lines 3120) described herein may include such sensors and be similarly monitored. Both the bag sensors 4312A, B and jaw position sensor(s) 4309 may be compared for sake of redundancy. In the event that the output signal of the bag positions sensors 4312A, B is unexpected based on the output signal received from a jaw position sensor(s) 4309, the control system 15 may generate a notification (e.g. message on a user interface 6100). The control system 15 may also place the system 10 in a standby state until serviced.

All wiring, cables, pneumatic lines, other lines, etc., in the bag retainer housing 4314 may be bundled together and passed through a gland 4342 defined in the bag retainer housing 4314 (see, e.g., FIG. 69A). A bundle sheath 4344A may be provided around the portion of the wiring, cables, pneumatic lines, etc. disposed outside of the bag retainer housing 4314 to facilitate wipe down and cable routing. In various embodiments, the bundle sheath 4344A may be routed into the actuator housing 4328 through a gland 4342. The constituent lines in the bundle are then routed through the actuator housing 4328 and joined by any wiring, cables, etc. for the rotary actuator 4334. All lines may exit the respective upstream or downstream displacement assembly 4302, 4304 via a gland 4342 into another bundle sheath 4344B. The bundle sheath 4344B may extend to the wall 4301 of the processing compartment 4300 and exit the processing compartment 4300 through a respective compartment wall gland 4346. Such bundling and gland arrangements may be used for other actuators, sensors, and assemblies described herein.

Referring now primarily to FIG. 72A-C, to help ensure that the bag displacement assembly 2618 is particularly compact, the displacement stages 4318A, B may be arranged to nest when brought in close proximity. As shown, the base bodies 4340 of each bag retainer assembly 4306 include cooperating shapes which allow the two base bodies 4340 to be displaced directly abreast one another. The cantilevered arm 4348 of the downstream displacement assembly 4304 may provide clearance for the housing 4314 of the bag retainer assembly 4306 on the upstream displacement assembly 4302 when the base bodies 4340 are so positioned. Thus, the bag retainer assemblies 4306 may be at substantially even height while still allowing the bag displacement assembly 2816 to be relatively compact.

The linear guides 4316A, B may be positioned in substantially the same plane and may be mounted to a wall of the processing chamber 4300. The displacement stage 4318B of the upstream displacement assembly 4302 includes a main portion 4350 (best shown in FIG. 68B) which interfaces with the linear slide 4316B and a riser 4352 which extends past the superiorly disposed linear guide 4316A. The riser 4352 may be the portion of the displacement stage 4318A defining the actuator housing 4328. As shown in FIG. 72B, the riser 4352 may have a profile which accepts the actuator housing 4328 of the downstream displacement assembly 4304 when the displacement stages 4318A, B are directly abreast one another.

Additionally, the surface of the riser 4352 most proximal the linear guide 4316A may include one or more flute or recess 4354. The displacement stage 4318B for the downstream displacement assembly 4304 is shown with a main body 4358. The portion of the displacement stage 4318B defining the actuator housing 4328 is coupled to a side of the main body 4358. As best shown in FIG. 72C, the flutes 4354 accept any structures extending proud of the face of the main body 4358 (e.g. fasteners). Thus, the main body 4358 of displacement stage 4318B may be displaced to a position intermediate linear guide 4316A and the riser 4354 of displacement stage 4318A.

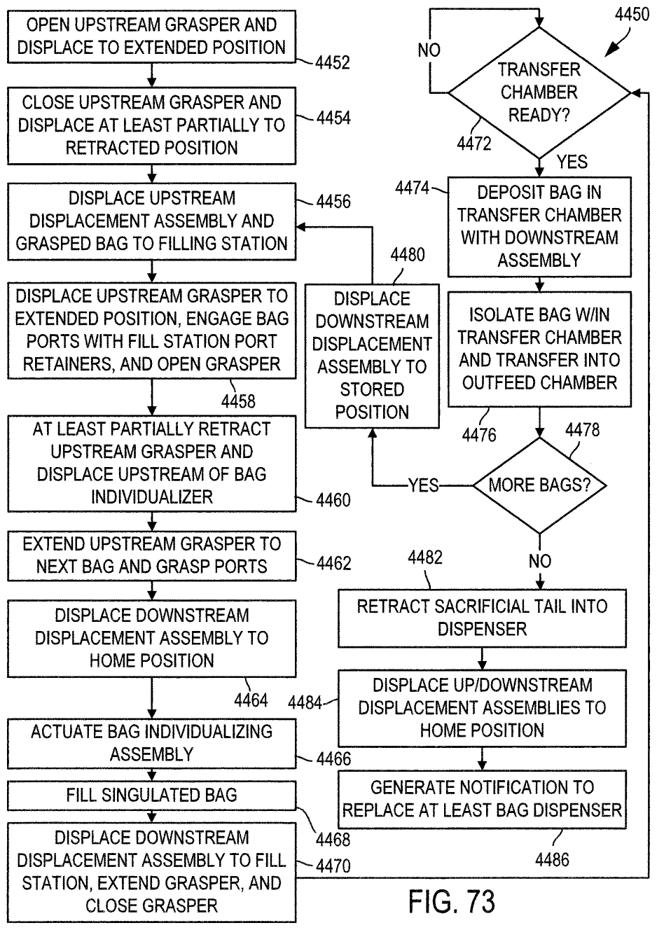

Referring now to FIG. 73, a flowchart 4450 detailing a number of example actions which may be executed to advance bags 26 through a processing chamber 4300 is depicted. The flowchart 4450 begins with a fresh dispenser 5000 loaded into the system 10 with a bag 26 retained in a bag presenter 5004 of the dispenser 5000. As shown, the bag retainer assembly 4306 of the upstream displacement assembly 4302 may be actuated (e.g. pneumatically) to an open or spread apart position in block 4452. The bag retainer assembly 4306 may also be displaced to an extended position in block 4452. In block 4454, the bag retainer assembly 4306 of the upstream displacement assembly 4302 may then be closed around ports 392 of the bag 26 at the bag presenter 5004 of the dispenser 5000 (see, e.g. FIG. 77). The bag retainer assembly 4306 may also be displaced at least partially to a retracted position in block 4452.

Where one or more bag sensor 4312A, B (see, e.g., FIG. 68B) is included in the housing 4314 for a bag retainer assembly 4306, the control system 15 may analyze the output signal from the bag sensor(s) 4312A, B to ensure the ports 392 are captured in place within the bag retainer assembly 4306. The output of any jaw position sensor(s) 4309 may also be analyzed. This may be done any time a bag 26 is grasped by a bag retainer assembly 4306 including bag sensor(s) 4312A, B and or jaw position sensor 4309. The control system 15 may analyze the output signal for the bag sensor(s) 4312A, B after grasping the ports 392, after displacement of the bag retainer assembly 4306 once the ports 392 are grasped, or at both of these time points. The control system 15 may confirm the ports 392 are grasped via the output signal from the jaw position sensor(s) 4309. Likewise, any time a bag retainer assembly 4306 hands off a bag 26 to another bag retainer 4306, the control system 15 may analyze the output signal for the bag sensor(s) 4312A, B after the bag retainer assembly 4306 releases the ports 392 and displaces away from the other bag retainer 4306. The control system 15 may confirm the ports 392 have been released by checking the output signal of the jaw position sensor(s) 4309. The control system 15 may verify that the output signal(s) of the bag sensor(s) 4312A, B indicate ports 392 are no longer present in the bag retainer assembly 4306. In the event that ports 392 are unexpectedly absent or present, a notification may be generated by the control system 15.

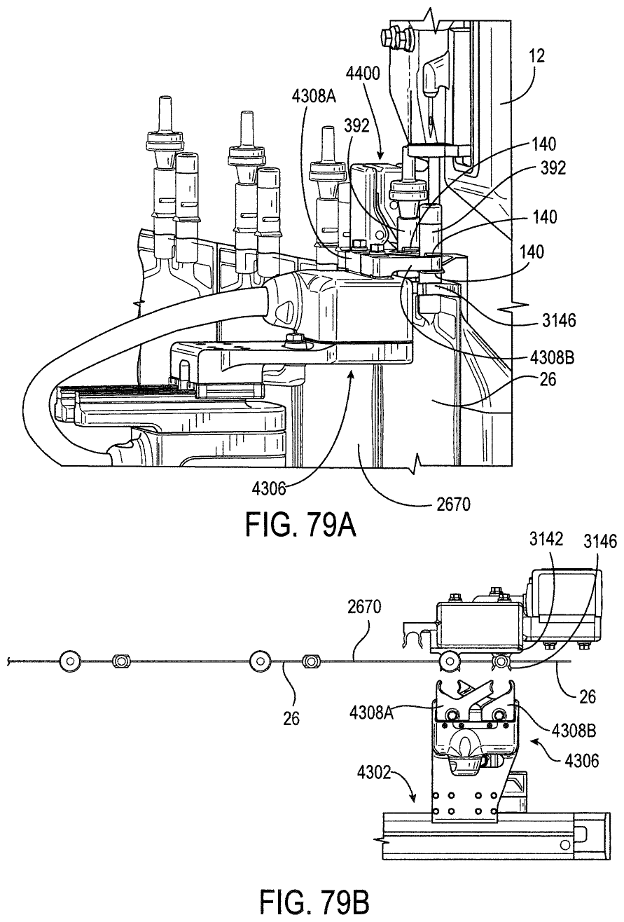

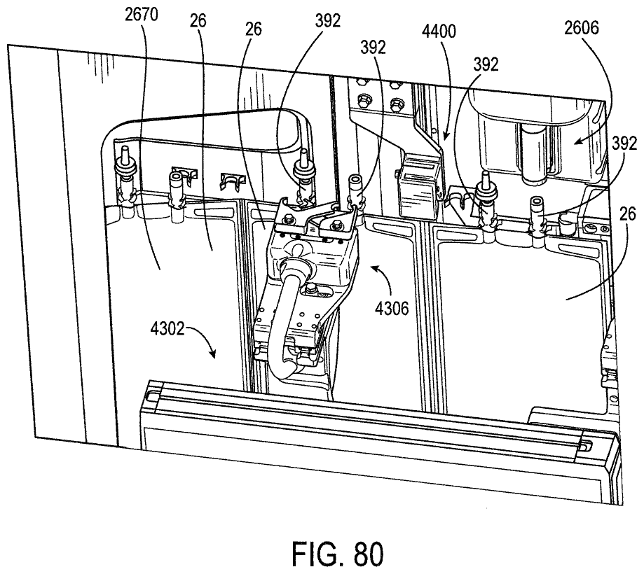

Still referring to FIG. 73, in block 4456, the upstream displacement assembly 4302 and grasped bag 26 may be displaced to a fill station 2600 (see, e.g., FIG. 78). The bag retainer assembly 4306 of the upstream displacement assembly 4302 is displaced to the extended position in block 4458. The ports 392 of the bag 26 may be engaged with the port retainers 3146 on the fill displacement stage 3142 of the fill station 2600 in block 4458 (see, e.g. FIG. 79A). The bag retainer assembly 4306 of the upstream displacement assembly 4302 may also be opened in block 4458. In block 4460, the bag retainer assembly 4306 of the upstream displacement assembly 4302 may be at least partially retracted (see, e.g., FIG. 79B) and displaced upstream of a bag individualizer assembly 4400 (see, e.g., FIG. 80). The bag retainer assembly 4306 of the upstream displacement assembly 4302 may be displaced to an extended position in block 4462. The bag retainer assembly 4306 may be closed about the ports 392 of the next bag 26 in the chain 2670 in block 4462 as well (see, e.g., FIG. 81B).

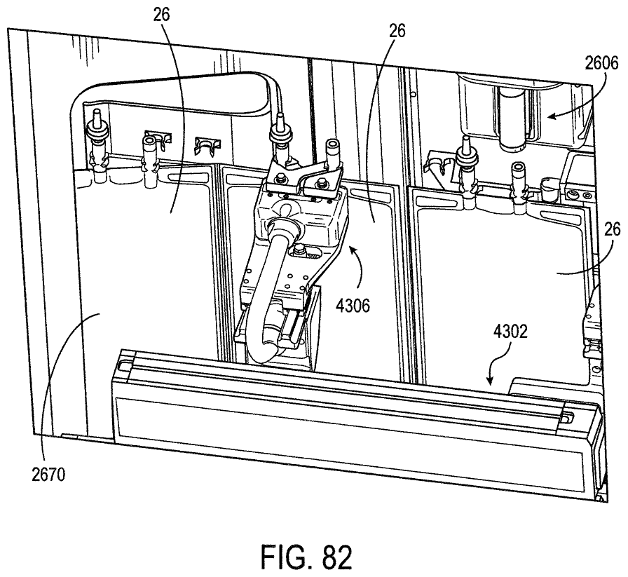

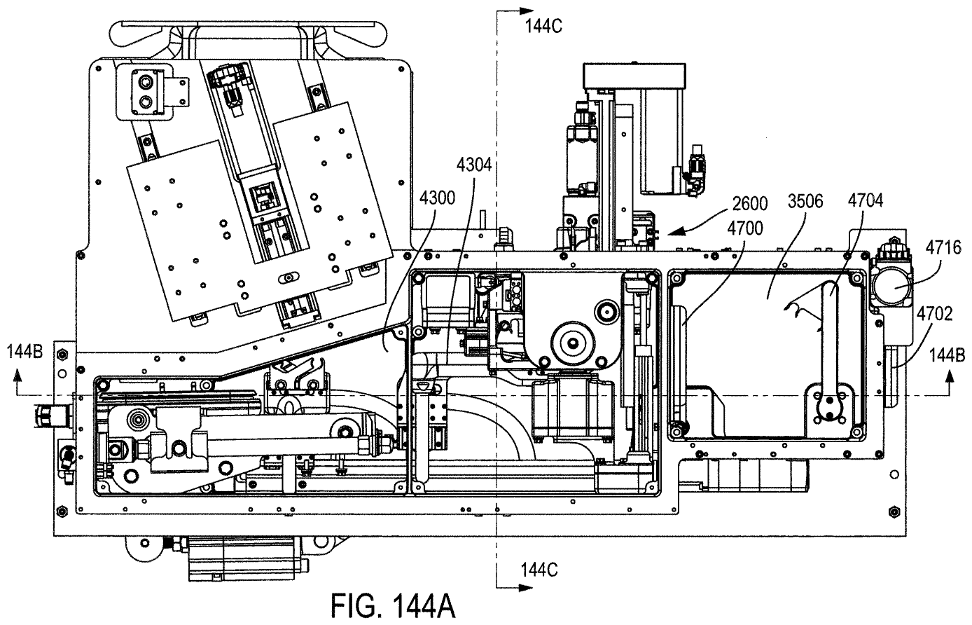

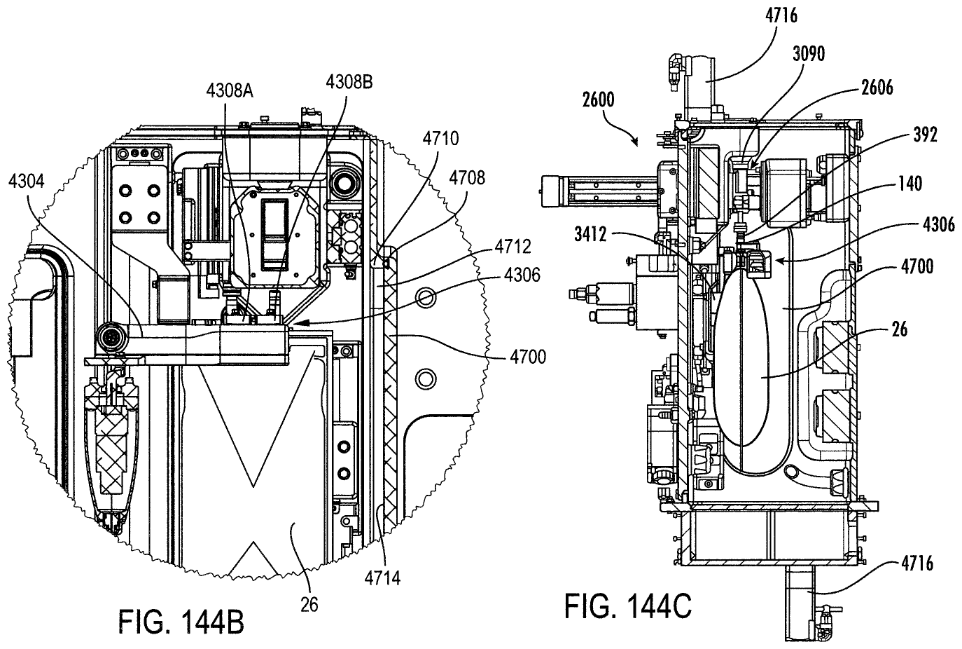

With a lead bag 26 of the chain 2670 retained at the fill station 2600 and the next bag 26 held by the bag retainer assembly 4306 of the upstream displacement assembly 4302, the downstream displacement assembly 4304 may be displaced to a home position in block 4464. The lead bag 26 may be separated from the chain 2670 in block 4466 by actuation the bag singulating assembly 4400 (see, e.g., FIG. 81B-82). The separated bag 26 may be filled at the fill station 2600 in block 4468. The downstream displacement assembly 4304 may be displaced such that its bag retainer assembly 4306 is aligned with the ports 392 of the filled bag 26 on the fill displacement stage 3142 in block 4470. The bag retainer assembly 4306 of the downstream displacement assembly 4304 may be also displaced to an extended state and closed about the ports 392 of the filled bag 26 in block 4470 (see, e.g., FIG. 144B).

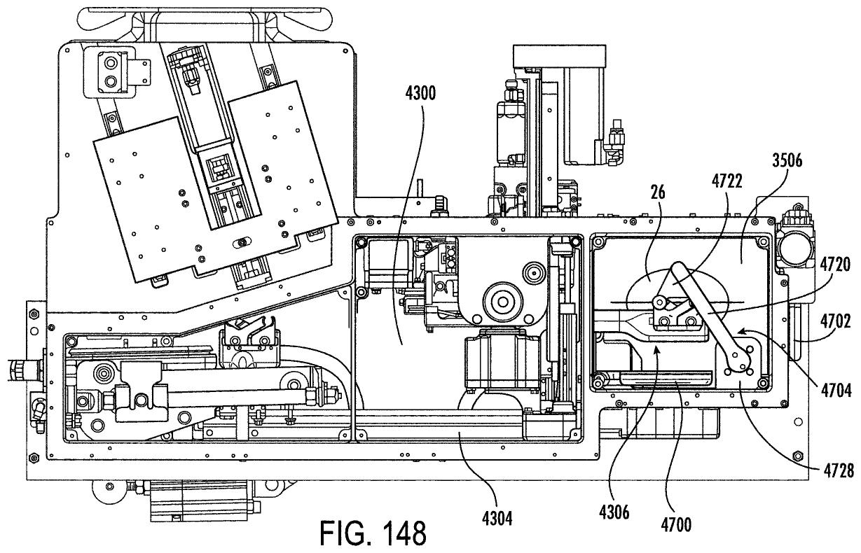

A transfer chamber 3506 may be positioned between the processing compartment 4300 and an outfeed compartment 4202 of the system 10. The transfer chamber 3506 may act as an airlock between the processing compartment 4300 and the outfeed compartment 4202. The transfer chamber 3506 may be opened to either the processing compartment 4300 or outfeed compartment 4202. Each time the transfer chamber 3506 is opened at least one recovery condition may be required to be met before it is opened again. For example, a predefined number of air exchanges through the transfer chamber 3506 may be required to occur before the control system 15 permits the transfer chamber 3506 to be accessed again. Set points for any characteristics relating to any other environmental control set point described herein may also be imposed as part of a set of recovery criteria. Alternatively, the control system 15 may permit the transfer chamber 3506 to establish communication with the outfeed chamber 4202 without recovery condition(s) being met, but inhibit communication of the transfer chamber 3506 with the processing compartment 4300 until the recovery condition(s) is/are met. When the transfer chamber 3506 is ready in block 4472, the bag retainer assembly 4306 of the downstream displacement assembly 4304 may be displaced into the transfer chamber 3506 in block 4474 (see, e.g., FIG. 145). The filled bag 26 may also be deposited in the transfer chamber 3506 in block 4474 (see, e.g., FIG. 148).

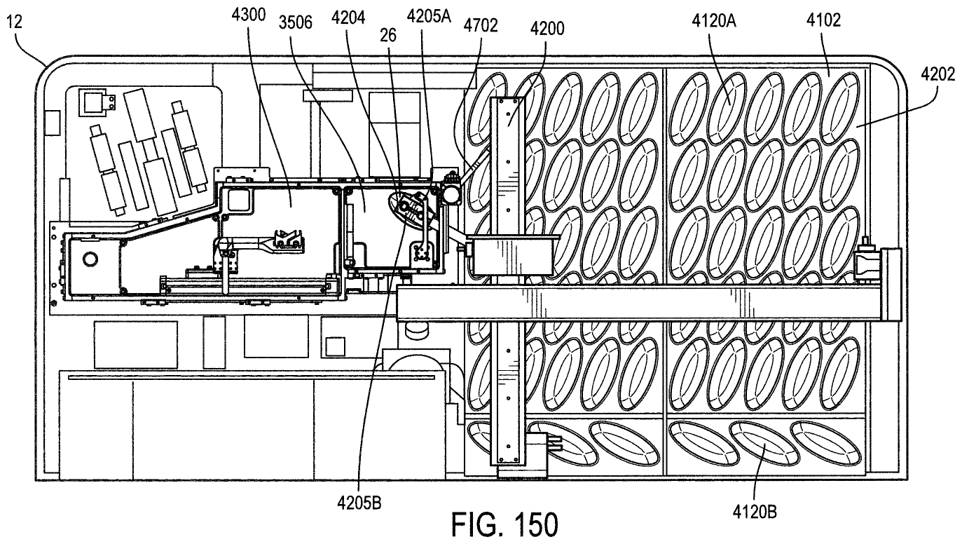

The filled bag 26 may be isolated in the transfer chamber 3506 in block 4476 (see, e.g., FIG. 149). The downstream displacement assembly 4304 may be displaced out of the transfer chamber 3506 (e.g. to the home position) to facilitate isolation of the filled bag 26. The filled bag 26 may also be displaced into the outfeed compartment 4202 in block 4476. A gantry assembly 4200 may for example, collect the bag 26 from the transfer chamber 3506 and displaced it to the outfeed compartment 4202 (see, e.g., FIGS. 150-151).

The control system 15 may write information relating to environmental control of the processing chamber 4300 and transfer chamber 3506 to a batch record for that particular bag 26. For example, data from the period of time before a bag 26 is exposed to the outfeed compartment 4202 may be included in the batch record for that bag 26. Alternatively or additionally, an indication that environmental control of the processing compartment 4300 and transfer chamber 3506 conformed to environmental control targets may be written to the batch record (e.g. as a pass/fail). The batch record may also include an indication that any recover conditions imposed on the transfer chamber 3506 have been conformed to. In the event environmental control of the processing compartment 4300 or transfer chamber 3506 is in breach of predefined control requirements, data relating to the offending control set point or requirement (e.g. particulate, pressure, air flow related information) may be written to the batch record.

Still referring to FIG. 73, if, in block 4478, there are additional bags 26 to be filled, the downstream displacement assembly 4304 may be displaced to a stored position in block 4480. In some embodiments, the stored position may be a position in which the downstream displacement assembly 4304 extends at least partially into the transfer chamber 3506. This may assist in keeping the system 10 as compact as possible. Where part of the downstream displacement assembly 4304 is disposed in the transfer chamber 3506 in the stored position, the control system 15 may ensure any recovery condition(s) are met before commanding displacement of the downstream actuation assembly 4304 to the stored position. The flowchart 4450 may then return to block 4456.

If, in block 4478, all consumable bags 26 have been filled and the dispenser 5000 is emptied of consumable bags 26, the sacrificial tail 2683 of the bag chain 2670 may be retracted into the dispenser in block 4482. Where the sacrificial tail 2683 includes a sacrificial bag or ports 392, the bag retainer assembly 4306 of the upstream displacement assembly 4302 may displace the sacrificial tail 2683 toward the dispenser 5000. The bag retainer assembly 4306 of the upstream displacement assembly 4302 may then open to release the sacrificial tail 2683. A tensioner assembly 5100 (see, e.g., FIGS. 41A-B) within the dispenser 5000 may pull the sacrificial tail 2683 into the dispenser 5000. Alternatively, the bag retainer assembly 4306 of the upstream displacement assembly 4302 may be actuated to advance ports 392 of the sacrificial tail 2683 into retained engagement with the port retainers 5068 (see, e.g., FIG. 38B) of the dispenser's 5000 bag presenter 5004. It should be noted that as bags 26 are consumed, any bag 26 currently forming the end of the chain 2670 may be re-engaged with the bag presenter 5004 port retainers 5068. This may allow the removable cover assembly 5002 to be re-engaged with the housing 2682 so that the dispenser 5000 may be removed or swapped out (e.g. with a dispenser of bags 26 containing a different concentrate).

Still referring to FIG. 73, with the sacrificial tail 2683 (or some leading bag in the chain 2670) stowed in the dispenser 5000, the upstream and downstream displacement assemblies 4302, 4304 may be displaced to home positions in block 4484. The removable cover assembly 5002 may also be reinstalled on the dispenser 5000 to seal the dispenser 5000. A notification may be generated for display on the user interface in block 4486. The notification may indicate that the dispenser 5000 is empty and needs to be replaced (or is ready to be exchanged with a different dispenser 5000). The notification may include text, graphics, and/or animations, and may be accompanied by audio output by at least one speaker.

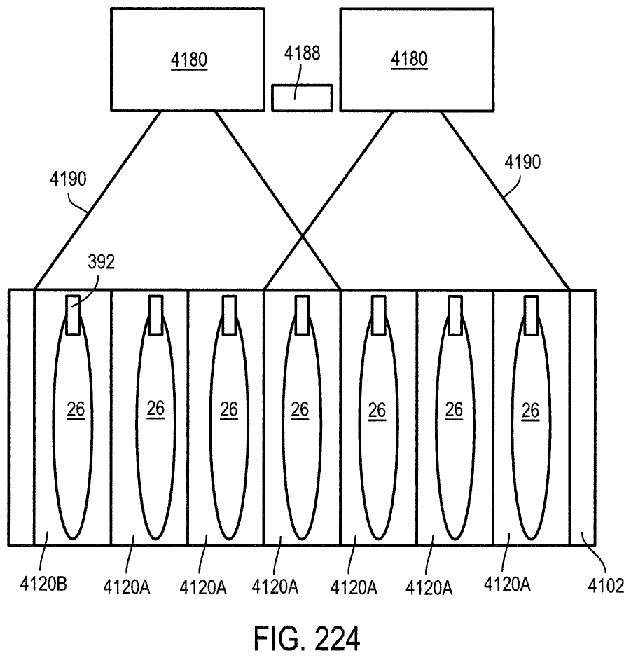

The control system 15 may also confirm space is available in the outfeed assembly 4100 (see, e.g. FIG. 221) in block 4478. In such embodiments, the control system 15 may keep a table or other record of which receptacles 4120A, B in an outfeed assembly 4100 are populated by bags 26. Alternatively, one or more imager 4180 (see, e.g. FIG. 224 and associated description) and images of the receptacles 4120A, B may be analyzed to verify space is present. In the event space is available in both acceptable bag receptacles 4120A, and rejected bag receptacles 4120B, the downstream displacement assembly 4304 may be displaced to a stored position in block 4480 and bags 26 may continue to be filled. If the amount of open receptacles 4120A, B for each respective type of receptacle 4120A, B is below a predefined threshold (e.g. <1), a notification may be generated and filling of bags 26 may be halted. The notification may instruct appropriately credentialed staff to empty the outfeed assembly 4100. Once emptied (and optionally verified by one or more receptacle sensor such as an imager 4180), the filling of bags 26 may resume and the flowchart 4450 may return to block 4456 after passing through block 4480.

In some implementations, the control system 15 may also check to see if the number of bags 26 specified in a current order has been met in block 4478. If less than the ordered number of bags 26 have been filled, the control system 15 may continue to orchestrate filling of bags 26 and the flowchart 4450 may return to block 4456 after passing through block 4480. In the event that the order has been satisfied, filling of bags 26 may be paused. In some embodiments, the lead bag 26 may be reengaged with the port retainers 5068 of the bag presenter 5004 as described above. In other embodiments, the system 10 may simply continue filling of bags 26 until all bags 26 in the dispenser 5000 have been consumed.

Referring now to FIG. 74, an example embodiment of a bag individualizer assembly or bag singulation assembly 4400 is depicted. As shown, the bag individualizer assembly 4400 includes a displaceable stage 4404. An exemplary splitter assembly 4406 is mounted to the displaceable stage 4404. The displacement stage 4404 may be displaced translationally through a displacement range by a singulation actuator 4402 from a raised position to an end of stroke position. The singulation actuator 4402 may define a guide for the displacement stage 4404. The singulation actuator 4402 may include a motor which drives a leadscrew or ball screw which is engaged with the displacement stage 4404. Alternatively, a pneumatic actuator may be used. The singulation actuator 4402 may include a position sensor 4408 which may output a signal indicative of the location of the displacement stage 4404 along its displacement range. The control system 15 of the system 10 may monitor the signal from the position sensor 4408 and commands to the singulation actuator 4402 may be based, at least in part, on the output from the position sensor 4408. A linear potentiometer may be included and a wiper may be included on the displacement stage 4404. Alternatively, a motor encoder may be used as the position sensor 4408. The singulation actuator 4402 may be disposed outside of the processing chamber 4300 and may transmit motion into the processing chamber 4300 via a sealed interface.

Referring now to FIGS. 75A-C, splitter assemblies 4406 may be driven into the coupling region 2677 between bags 26 on a chain 2670 to separate or individualize a bag 26 from the chain 2670. The splitter assembly 4406 may include a blade which cuts through the coupling region 2677 along the weakened region 2678 in some examples. Thus, bags 26 may be cut from the chain 2670 to singulate bags 26. Preferably, the splitter assembly 4406 is devoid of blades or other sharp bodies. In such embodiments, the splitter assembly 4406 may include a plow or wedge which may be driven into the weakened region 2678. Splitter assemblies 4406 incorporating a set of rollers which create a tearing action along the weakened region 2678 as shown in FIGS. 75A-C may also be used.

As shown in FIGS. 75A-C, example splitter assemblies 4406 may include a first body 4410A and second body 4410B which couple to one another (e.g. via a fastener or in any other suitable fashion). The first body 4410A may be integral to the stage 4404 or a separate component coupled to the stage 4404. A feed roller 4412A, B may be coupled into each of the first and second bodies 4410A, B. The feed rollers 4412A, B may be mounted on bearings 4416A, B and may be formed of a low friction coefficient material such as PTFE. A set of separating rollers 4414A, B may be captured between the first and second body 4410A, B. The separating rollers 4414A, B may share a common bearing 4416C and may not be coupled to one another. Thus, the separating rollers 4414A, B may counter rotate relative to one another.

As best shown in FIG. 75C, when assembled, a splitter assembly 4406 may include a leading end 4418A and a trailing end 4418B. Each of the first and second bodies 4410A may include a ramped surface 4420. The ramped surfaces 4420 may be arranged such that the first and second bodies 4410A, B taper thinner as distance toward the leading end 4418A decreases. Thus, the leading end 4418A of the splitter assembly 4406 may form a guide in the shape of the Latin character “V”. As the leading end 4418A is displaced toward a chain 2670 of bags 26, the guide formed by the ramped surfaces 4420 allows significant tolerance in the position of the top of the chain 2670. As the chain 2670 enters the splitter assembly 4406 through the leading end 4418A, the ramped surfaces 4420 may direct the chain material 2670 generally into a feed channel 4422 positioned in a central region of the splitter assembly 4406. The feed rollers 4412A, B may assist in directing the chain 2670 material into the feed channel 4422. The feed rollers 4412A, B may also assist in mitigating particulate generation.

Referring now also to FIG. 74, the feed channel 4422 may furcate into a first channel branch 4424A, and second channel branch 4424B. The first and second channel branch 4424A, B may extend substantially parallel to one another on opposing sides of the separating rollers 4414A, B. The first channel branch 4424B may be defined by a recess into the upstream side 4426A (that most proximate the dispenser 5000) of the splitter assembly 4406. The second channel branch 4424B may be defined by a recess into the opposite, downstream side 4426B of the splitter assembly 4406. As the splitter assembly 4406 is displaced into a chain 2670, the upstream portion of the chain 2670 may be routed through the first channel branch 4424A. This may displace the upstream portion of the chain 2670 in a first direction generally perpendicular to the plane of the unfilled bags 26 in the chain 2670. The bag 26 on the end of the chain 2670 may be routed through the second channel branch 4424B. This may displace the lead bag 26 on the chain 2670 in a second direction opposite the first. The opposite displacement directions of the lead bag 26 and the adjacent upstream bag 26 may generate tearing action which separates the bag 26 on the end of the chain 2670 as the splitter assembly 4406 is driven through the chain 2670. The tear may propagate along the tear guide or weakened region 2678 between the bags 26. As mentioned elsewhere herein, an end of the weakened region 2687 may be pre-separated or pre-torn to facilitate tearing. Counter rotation of the separating rollers 4414A, B may assist in feeding the upstream bag 26 on the chain 2670 and the end bag 26 into the respective first and second channel branches 4424A, B. This tearing type splitting action may be particularly desirable as it may mitigate potential for the splitter assembly 4406 to snag as it is displaced through the chain 2670. The absence of blades may also help to ensure that bags 26 are not compromised during separation.

The first and second channel branches 4424A, B may each extend across 70-90% of the width of the splitter assembly 4406. This may support the weakened region 2678 of the bags 26 being introduced over a wide range of positions without deleterious effect on the separation of the lead bag 26 from the chain 2670. Thus, the splitter assembly 4406 may be relatively tolerant to misalignment of the chain 2670 relative to the singulation assembly 4400.

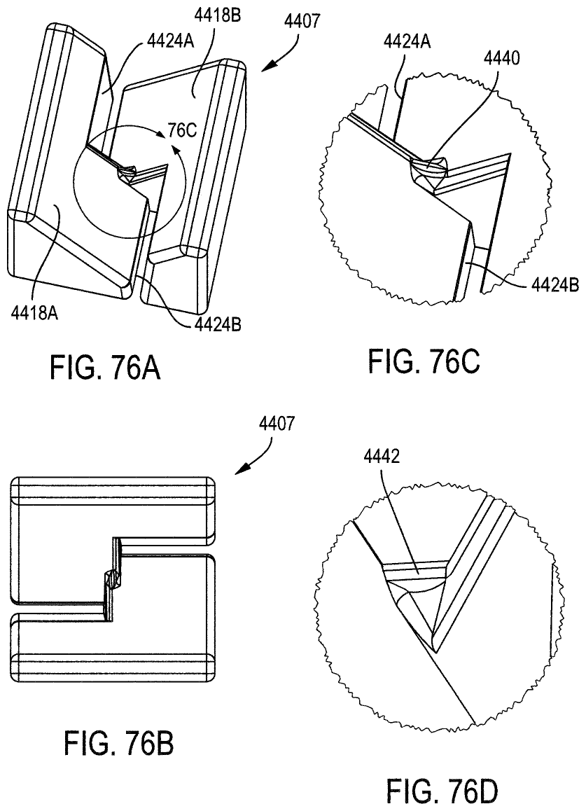

Referring now to FIGS. 76A-D, a number of exemplary splitters 4407 are depicted. As shown, the example splitter 4407 of FIGS. 76A-C is formed as a single monolithic component which includes a splitting wedge 4440. The splitting wedge 4440 may be replaced by a blade 4442 in certain examples. Where a blade 4442 is used, a ceramic blade may be preferable. As shown, the splitter 4407 may include ramped surfaces 4418A, B which may direct a chain 2760 of bags 26 into the splitting wedge 4440. The splitter 4407 may include a first channel branch 4424A and second channel branch 4424B to direct bags 26 in opposite directions as the splitter 4407 is actuated through the chain 2670. Splitters 4407 may be coupled to a displacement stage 4404 similar to that shown in FIG. 74.

Referring now to the progression of FIGS. 77-82, a chain 2670 of bags 26 is shown being displaced into a processing chamber 4300 with a lead bag 26 of the chain 2670 being removed by a singulation assembly 4400. A number of components of the system 10 are not depicted or simplified in FIGS. 77-82 for ease of illustration. As shown in FIG. 77, the bag retention assembly 4306 of an upstream displacement assembly 4302 may be displaced to a bag 26 retained in a bag presenter 5004 of a dispenser 5000. The jaws 4308A, B of the bag retention assembly 4306 may be closed about the ports 392 of the bag 26 retained on the bag presenter 5004.

The upstream displacement assembly 4302 may then be displaced to the fill station 2600 as shown in FIG. 78. Since the lead bag 26 is held by the bag retention assembly 4306 of the upstream displacement assembly 4302, the chain 2670 of bags 26 may be spooled out of the dispenser 5000 as this occurs. The bag retention assembly 4306 of the upstream displacement assembly 4302 may be aligned with the port retainers 3146 on a fill displacement stage 3142 of the fill station 2600. In some examples, the fill displacement stage 3142 may be lowered by a bag displacement actuator 3144 (see, e.g., FIG. 131) such that the port retainers 3146 are at a height below projections (e.g. ribs 140) of ports 392 held in the jaws 4308A, B of port retention assembly 4306. The bag retention assembly 4306 may be driven to an extended position as depicted in FIG. 79A. This may press the ports 392 of the lead bag 26 into engagement with the port retainers 3146 on the fill displacement stage 3142. As shown, the port retainers 3146 may preferably be passive. The ribs 140 on the ports 392 may ensure that the bag 26 is held in place. Though the jaws 4308A, B are shown gripping between the ribs 140, the jaws 4308A, B may be directly below the rib 140 most proximate the bag 26 in other examples. The space intermediate the ribs 140 would then be pressed into the port retainers 3146. The bag retainer assembly 4306 of the upstream displacement assembly 4302 may then be retracted from the fill displacement stage 3142 as shown in FIG. 79B.

The upstream displacement assembly 4302 may be displaced such that the jaws 4308A, B of its bag retention assembly 4306 are aligned with the ports 392 of the bag 26 adjacent the lead bag 26 as shown in FIG. 80. The bag retention assembly 4306 may then be extended and the jaws 4308A, B of the bag retention assembly 4306 may be closed about the ports 392 as shown in FIG. 81A. This may place and hold the coupling region 2677 between the lead bag 26 and the adjacent bag 26 in alignment with the splitter assembly 4406 (or splitter 4407) of the bag individualizing assembly 4400. The control system 15 may command the singulation actuator 4402 to drive the splitter assembly 4406 through is displacement range. As best shown in FIG. 81B, the splitter assembly 4406 may separate the lead bag 26 from the chain 2670 along the tear guide or weakened region 2678 (see, e.g., FIG. 81B) between the bags 26. The material of the lead bag 26 may exit the second channel branch 4424B of the splitter assembly 4406 while the material of the chain 2670 is directed into the first channel branch 4424A. The lead bag 26 is shown separated from the chain 2670 in FIG. 82.

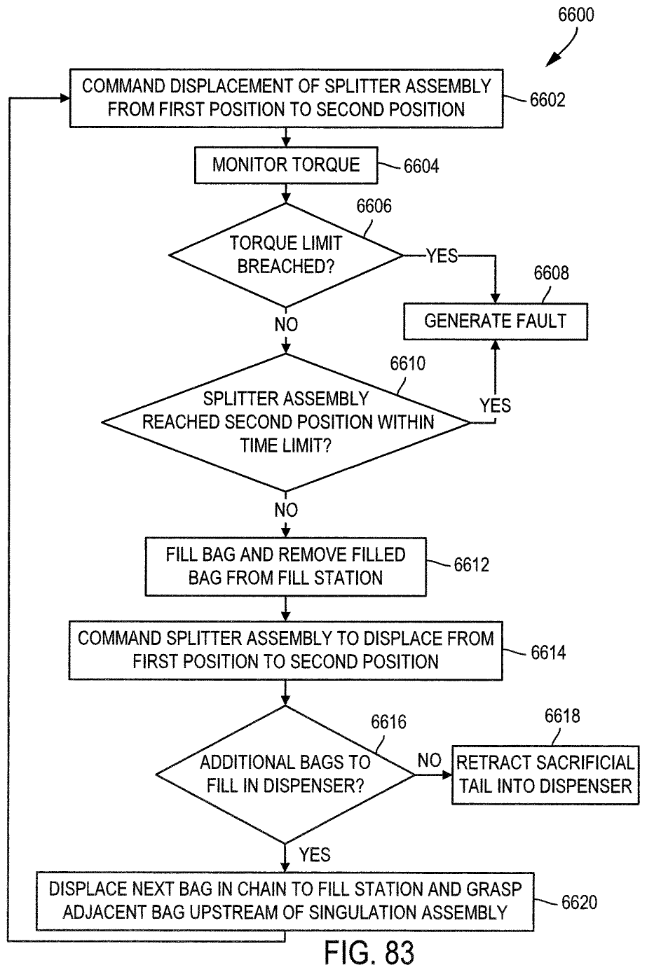

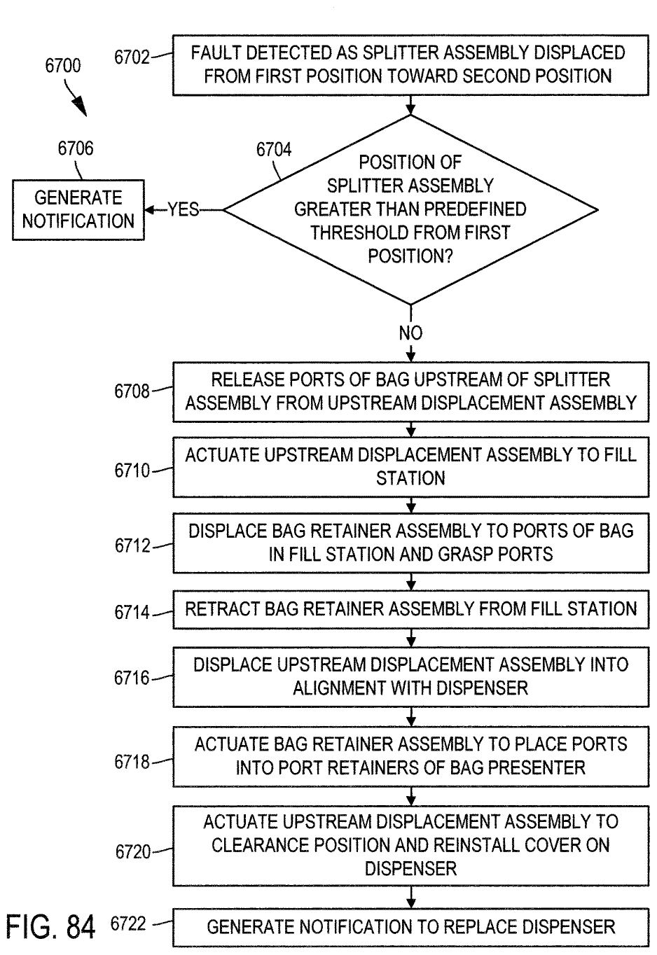

Referring now to FIG. 83, a flowchart 6200 depicting a number of example actions which may be executed to separate bags 26 from a chain 2670 at a singulating assembly 4400 is depicted. With a bag 26 in place at a fill station 2600 and a grasper 4306 holding the ports 392 of the adjacent bag 26 (see, e.g., FIG. 81A), the control system 15 may command displacement of a splitter assembly 4406 from a first position to a second position in block 6202. Though described in relation to the splitter assembly 4406 any splitter 4407 could be used. The control system 15 may monitor the singulation actuator 4402 as the splitter assembly 4406 displaces. In the example, the control system 15 monitors motor torque (which may be determined based on the current flow through the motor) in block 6604. If a torque limit (or current limit) is breached in block 6606, a fault may be generated in block 6608 (further described in relation to FIG. 84). The control system 15 may also monitor the travel of the splitter assembly 4406. The control system 15 may analyze data from a position sensor 4408 such as an encoder of the singulation actuator 4402 to determine the position of the splitter assembly 4406. If, in block 6610, the splitter assembly 4406 does not displace to the second position within a time limit, a fault may be generated in block 6608. If the splitter assembly 4406 reaches the second position within an appropriate time in block 6610, the bag 26 at the fill station 2600 may be filled in block 6612. The bag 26 may also be removed from the fill station 2600 in block 6612. The control system 15 may command the splitter assembly 4406 to return to the first position in block 6614. If, in block 6616, there are no further consumable bags 26 in the chain 2670, the sacrificial tail 2683 of the chain 2670 may be retracted into the dispenser 5000 in block 6618. The bag displacement assembly 2618 may, for example, grasp a portion of the sacrificial tail 2683 (e.g. a sacrificial bag) and displace it toward the outlet of the dispenser 5000. The bag 26 may then be released and a tensioning assembly 5100 of the dispenser 5000 may pull the sacrificial tail 2683 fully into the dispenser 5000. The sacrificial tail 2683 may also include ports in some examples, and these ports may alternatively be retained in place on the bag presenter 5004 of the dispenser 5000. If, in block 6616, there are additional consumable bags 26, the next bag 26 in the chain 2670 may be displaced to the fill station 2600 via the bag displacement assembly 2618 in block 6620. The adjacent bag upstream of the singulation assembly 4400 may also be grasped by the bag displacement assembly 2618 in block 6622. The flowchart 6600 may then return to block 6602.

The control system 15 may also write certain information collected as each bag 26 is singulated to a batch record for that bag 26 (and optionally the subsequent bag 26 on the chain 2670). For example, the control system 15 may use the torque and position data to determine a position of the splitter assembly 4406 where peak torque was recorded. Information related to the torque data may also be documented. For example, the maximum, average, and standard deviation for torque readings may be written to the batch record for the bag 26. The time taken to singulate the bag 26 and an indication of whether the bag 26 was successfully individualized may be written to the batch record of the bag 26 as well.

Referring now to FIG. 84, a flowchart 6700 detailing a number of example actions which may be executed in the event a fault is encountered during individualization of a bag 26 from a chain 2670 is depicted. As mentioned in relation to FIG. 83, a fault may be generated if sensor data from the singulating assembly 4400 is indicative a snag of the splitter assembly 4406 has occurred. In certain embodiments, this may be determined by the control system 15 if torque data from the singulation actuator 4402 exceeds a predefined threshold or exceeds a predefined threshold in a predefined range of positions. As shown, in block 6702, the fault may be detected by the control system 15 as the splitter assembly 4406 is actuated from the first position (e.g. a raised position) to a second position (e.g. an end of its displacement opposite the raised position). If, in block 6704, the output signal from the position sensor of the singulating assembly 4400 indicates the splitter assembly 4406 is more than a predefined distance from the first position, the control system 15 may generate a notification in block 6706. The notification may communicate the fault (e.g. via a user interface 6100) and instruct the user that a service event is necessary. The control system 15 may also enter an indication that singulation from the chain 2670 has failed in the batch record for that particular bag 26 in block 6704.

If, in block 6704, the output signal from the position sensor indicates the splitter assembly 4406 is less than the predefined distance from the first position, the ports 392 of the bag 26 upstream of the singulating assembly 4400 may be released by the upstream displacement assembly 4302 in block 6708. The control system 15 may also enter an indication that singulation from the chain 2670 has failed in the batch record for that particular bag 26 in block 6708. In block 6710, the upstream displacement assembly 4302 may be actuated into alignment with the fill station 2600. The bag retainer assembly 4306 may be displaced to the ports 392 of the lead bag 26 of the chain 2670 (which would be retained in port retainers 3146 of the fill station 2600) in block 6712. The control system 15 may also command the jaws 4308A, B of the bag retainer assembly 4306 into a closed state to grasp the ports 392 of the lead bag 26 in block 6712. The bag retainer assembly 4306 may be retracted in block 6714 removing the lead bag 26 from the fill displacement stage 3142. In block 6716, the control system 15 may orchestrate actuation of the upstream displacement assembly 4302 into alignment with the bag presenter 5004 of the dispenser 5000. The bag retainer assembly 4306 of the upstream displacement assembly 4302 may be actuated to drive the ports 392 of the lead bag 26 of the chain 2670 into engagement with the port retainers 5068 of the bag presenter 5004 in block 6718. In block 6720, the control system 15 may command actuation of the upstream displacement assembly 4302 to a clearance position. The removable cover assembly 5002 may also be reinstalled onto the dispenser 5000 in block 6720 (further described in relation to FIG. 47). In block 6722, a notification (e.g. user interface screen or animation) may be generated by the control system 15 instructing that the dispenser 5000 be removed and replaced.

Referring now to FIG. 85, the system 10 may include at least one bag chain orientation sensor 6730. The chain orientation sensor(s) 6730 may monitor the position of the chain 2670 as the chain 2670 is spooled out of a dispenser 5000. The output signal(s) for the at least one chain orientation sensor 6730 may be used to adjust the position of the lead bag 26 in the chain 2670 when the lead bag 26 is in place in the port retainers 3146 of the fill displacement stage 3142. The fill displacement stage 3142 may be actuated to displace the lead bag 26 until the data from the chain orientation sensor(s) 6730 indicates the angle of the chain 2670 from the dispenser 5000 to the fill displacement stage 3142 is within a predefined acceptable range. This may facilitate grasping of the bag 26 adjacent the lead bag 26 (upstream of the singulation assembly 4400) with the bag retainer assembly 4306 of the upstream displacement assembly 4302. It may also facilitate individualization of the lead bag 26 from the chain 2670 at a singulation assembly 4400. Any suitable bag chain orientation sensor 6730 arrangement may be used. In some embodiments, one or more set of beam break sensors detecting the top and bottom edges of the chain 2670 may be included. Alternatively, and as depicted in FIG. 85, the bag chain orientation sensor 6730 may be an imager. Such an imager may be mounted on the wall of the processing chamber 4300 on which the upstream and downstream displacement assemblies 4302, 4304 are disposed. The imager may also be mounted outside of the processing chamber 4300 (as shown) and have a line of sight into the interior of the processing chamber through a window 6732. Any imager type chain orientation sensor 6730 may have a field of view which encompasses at least a portion of the space where the chain 2670 extends to the fill displacement stage 3142. This space may be referred to herein as a chain infeed pathway. The field of view may include a region of the processing chamber 4300 in which ports 392 of a bag 26 adjacent the lead bag 26 are positioned prior to singulation of the lead bag 26. This may be a region between where the chain 2670 is introduced into the processing chamber 4300 and the fill displacement stage 3142.

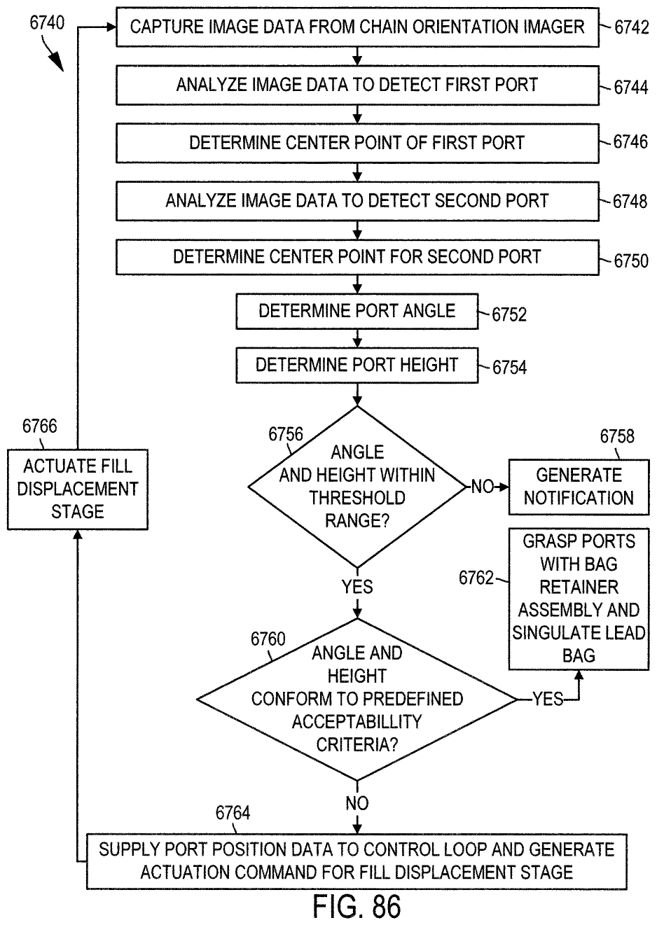

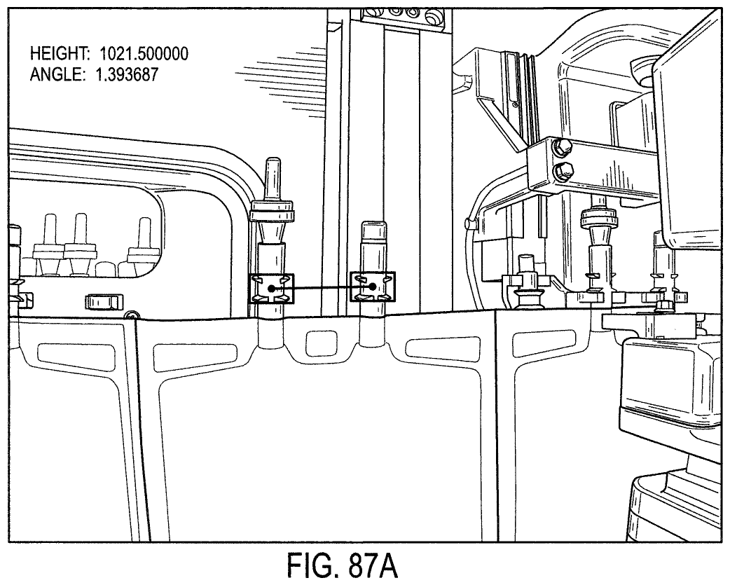

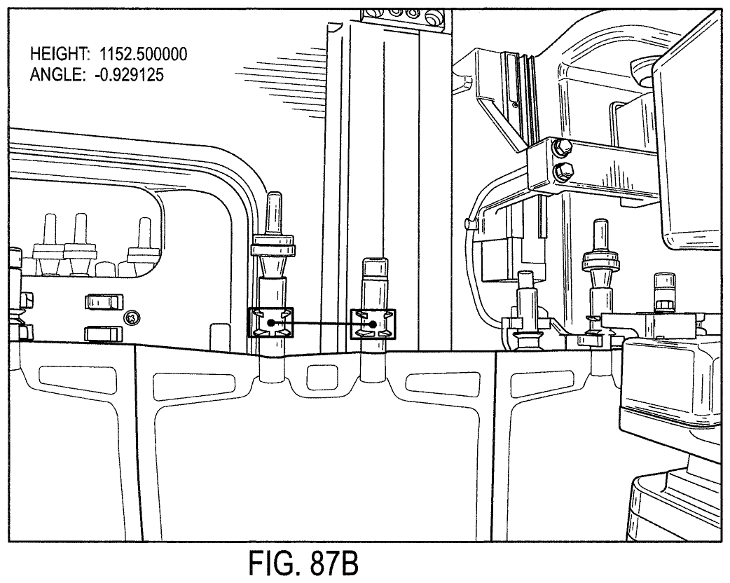

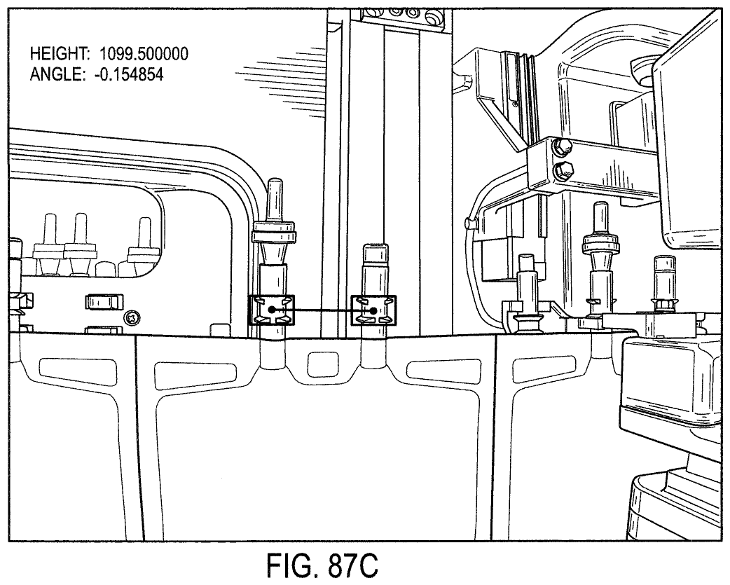

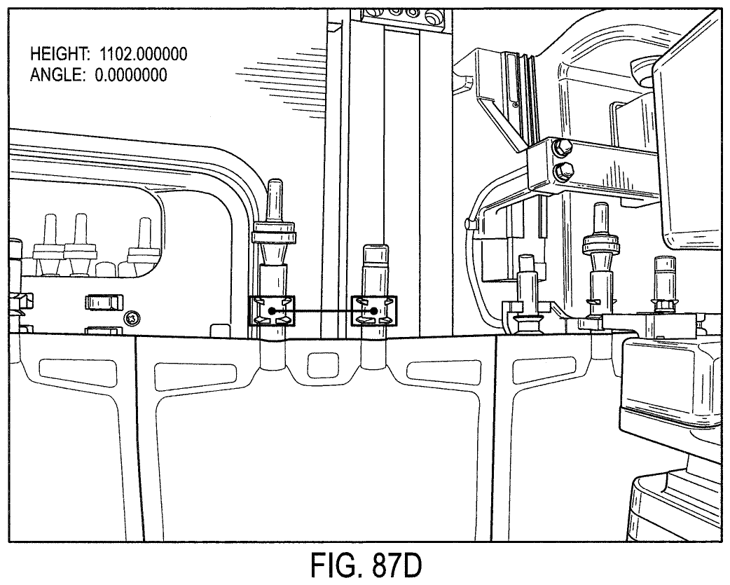

Referring now to FIG. 86, a flowchart 6740 depicting a number of example actions which may be executed to adjust the orientation of a chain 2670 of bags 26 spooling out of a dispenser 5000 is shown. For sake of example, position data for the chain 2670 is collected from an imager type chain orientation sensor 6730 in FIG. 86. Throughout the discussion of FIG. 86, reference is made to FIGS. 87A-D, a series of illustrative representations of images from an imager type chain orientation sensor 6730. Position data relating to the ports 392 may be determined by detecting the ports 392 within image data captured by an imager type chain orientation sensor 6730. The position data may be stored in memory and used to create a positional characterization for the chain 2670 by the control system 15. The positional characterization may include a height characterization aspect and a levelness characterization aspect.

With reference primarily to FIG. 86, in block 6742, image data from a chain orientation sensor 6730 may be captured. The image may include the portion of the chain 2670 including an expected location of the ports 392 of the bag 26 adjacent the lead bag 26 of the chain 2670. In block 6744, the control system 15 may analyze the image data to detect a first port 392 of the bag 26 adjacent the lead bag 26 in the captured image. A center point 6745A of the detected first port 392 may be determined by the control system 15 in block 6746. The control system 15 may also analyze the image data to detect a second port 392 of the 26 adjacent the lead bag 26 in block 6748. In block 6750, a center point 6745B of the detected second port 392 may be determined by the control system 15. The example depicted in the flowchart of FIG. 86 is for a chain 2670 of bags 26 each having a pair of ports 392. When a differing number of ports 392 are included, the image data may be analyzed to find each of the ports 392 in addition to a center point 6745A, B for each port 392. If no ports 392 are determined to be present in the captured image, a notification may be generated by the control system 15 (e.g. indicating a fault or error has occurred).

In various embodiments, the analysis may be a template matching analysis using template images of the respective ports 392. The template images may be collected from images of the ports 392 of a bag 26 adjacent the lead bag 26 within an example processing chamber 4300. The ports 392 may be placed in a controlled position corresponding with their ideal location as a chain 2670 is spooled out of a dispenser 5000. In some examples, the template for each port 392 may be a masked template with pixels relevant to the ports 392 being isolated using the template mask.

Any suitable template matching analysis may be used by the control system 15. In certain examples, a linear spatial filtering based template matching analysis may be used. A square difference or normalized square difference analysis may be conducted by the control system 15. Alternatively a cross-correlation, normalized cross-correlation, correlation coefficient, or normalized correlation coefficient matching analysis may be utilized. In some embodiments, an image pyramid may be generated and employed to exclude regions of the captured image from the analysis in order to hasten processing time. A low-resolution version of the captured image may be compared to a low-resolution version of the template to attempt to quickly identify the likely location of the port 392. The corresponding region of the raw image may then be analyzed for matches to the template image. In certain embodiments, instead of using respective templates for each port 392, a single port template may be employed and thresholding may be used on the output of the matching analysis. Regions of the image data which match the template with a greater degree of correspondence than a predefined threshold may be determined to be port 392 detections by the control system 15. In various examples, an eigenspace template matching analysis with multiple templates of a port 392 (or each respective port 392) may be utilized. Port 392 templates may be at different distances from the imager and at different orientations relative to the imager (the different templates may be collected over the range of anticipated port 392 positions during system 10 operation). Alternatively, other object detection approaches may be employed. For example, a convolutional neural network trained on images of each port 392 may be utilized by the control system 15. The control system 15 may also use feature matching such as, but not limited to, brute-force matching or Fast Library for Approximate Nearest Neighbor (FLANN) based matching.

In block 6752, the angle of the ports 392 relative to a perfectly level orientation for a chain 2670 or a chain levelness characterization may be determined. Referring to FIG. 87A for example, in certain implementations where ports 392 are identified via template matching, a bounding box 6770 may be drawn around the matching portion of the captured image. The control system 15 may, for example, determine a set of coordinates defining the height and width for each bounding box. The Y-coordinates of corresponding locations of the bounding box for each port 392 and the distance 6772 between these corresponding locations may be used to determine the angle of the ports 392. The Y-coordinate difference or height difference, as well as the distance between each port 392 (X-coordinate difference), may be measured in pixels. The corresponding locations are referred to in example embodiments as center points (e.g. of the bounding boxes). Points corresponding to the location of common features or like regions on each port 392 (e.g. the most distal point of a specified protrusion on each detected port 392) could also be utilized as the corresponding locations. Any suitable corresponding test points on each detected port 392 may be used.

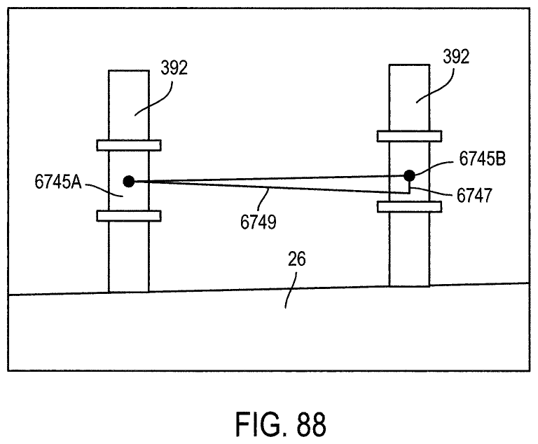

Small angle formula may, for example, be used to determine the tilt of the ports 392 as described in relation to FIG. 88. As shown in FIG. 86, a height position for the ports 392 may be determined in block 6754. Again referring to FIG. 87A, the Y-coordinates of corresponding locations on the respective bounding boxes 6770 for each port 392 may, for example, be averaged to determine a height position. Aspects of the positional characterization for the chain 2670 may be compared to various acceptability criteria. There may be a first set of acceptability criteria and a second set of acceptability criteria. If the positional characterization is in conformity with both, the chain 2670 may be deemed acceptably level. If at least one aspect of the positional characterization is in breach of the first acceptability criteria and none are in breach of the second acceptability criteria, an adjustment may be made to alter the positon of the chain 2670. If at least one aspect of the positional characterization is in breach of the second criteria, an error condition may be determined to exist.

With reference again to example illustrated in FIG. 86, if, in block 6756, the angle and/or height are outside of respective predetermined ranges, a notification may be generated in block 6758. The notification may, for example, be a screen for display on the user interface 6100 communicating a feed issue to the user. Otherwise, the control system 15 may check if the height and/or angle need to be adjusted. This may be done by comparing the determined height and angle to respective predefined acceptability criteria (e.g. a predefined height range and predefined angle range). For instance, if in block 6760, the height and angle conform to the acceptability criteria, the control system 15 may orchestrate grasping of the ports 392 with the bag retainer assembly 4306 of the upstream displacement assembly 4302 in block 6762. The control system 15 may also orchestrate actuation of the singulation assembly 4400 to individualize the lead bag 26 of the chain 2670 in block 6762.

If, in block 6760, the height and/or angle are in breach of acceptability criteria, position data (e.g. port angle) may be fed to a control loop (e.g. a P, PI, or PID loop) executed by the control system 15 in block 6764. The control loop may output a position target for the fill displacement stage 3142 and an actuation command for the bag displacement actuator 3144 may be generated. In block 6766, the fill displacement stage 3142 may be displaced via actuation of the bag displacement actuator 3144. The flowchart 6740 may then return to block 6742.

Referring primarily to FIGS. 87A-D, position data from images of the ports 392 may continue to be analyzed and fed to the control loop until the height and angle of the ports 392 achieve an orientation that is in conformity with acceptability criteria. For sake of example, the progression of FIGS. 87A-D depict an overshoot and correction to perfect levelness for the chain 2670. In certain implementations, the control system 15 may attempt to correct the angle to substantially 0° via actuation commands to the bag displacement actuator 3144. In some examples, the control system 15 may simply attempt to correct the angle to substantially 0° as long as the detected height is in an acceptable range. In alternative embodiments, the control system 15 may generate actuation commands to the bag actuator 3144 to both level the chain 2670 and adjust the height toward an ideal height position (e.g. without breaching a predefined range of angles having acceptable levelness).

Referring now to FIG. 88, a representational diagram showing a chained bag 26 being fed into a processing chamber 4300 at a slight angle is depicted. As shown, center points 6745A, B are depicted on each of the ports 392. These may be points corresponding to the center of the region matching a template for the ports 392. Center point 6745A may be understood to have coordinates (Xl, Yl), while center point 6745B may be understood to have coordinates (Xr, Yr). A height difference 6747 between the ports 392 may be determined by subtracting Yl from Yr. The spacing distance 6749 between the ports 392 may be determined by subtracting Xl from Xr. The height difference 6747 divided by the spacing distance 6749 should provide the tangent of the tilt angle between the ports 392. Relying on small angle approximation, the tangent function may be replaced with the angle itself. Thus, the angle between the ports 392 may be defined as height difference 6747 divided by the spacing distance 6749.

Though a chain orientation sensor 6730 may be included, alternative embodiments may displace the chain 6730 through a range of predetermined positions until the upstream displacement assembly 4302 is able to grasp the ports 392 of the bag 26 adjacent the lead bag 26. The control system 15 may command the bag retainer assembly 4306 of the upstream displacement assembly 4302 to grasp the ports 392 of the bag 26 adjacent the lead bag 26 after the lead bag 26 is retained on the fill displacement stage 3142. The jaw position sensors 4309 and bag sensors 4312A, B may be checked (further described in relation to FIGS. 69A-72A) by the control system 15 to determine whether the ports 392 were grasped. If the ports 392 are not detected in the jaws 4308A, B of the bag retainer assembly 4306, the control system 15 may command the fill displacement actuator 3144 to move the fill displacement stage 3142 to a predefined position. After displacement to this position, another attempt to grab the ports 392 may be commanded. If the ports 392 are not detected in the jaws 4308A, B, the control system 15 may command the fill displacement actuator 3144 to move the fill displacement stage 3142 to a second predefined position. Another attempt may be made to grasp the ports 392. The fill displacement stage 3142 may continue to be moved to additional predefined positions until the ports 392 are successfully grasped or until all positions in a list of predefined positions have been exhausted. The control system 15 may generate a notification once it is determined the ports 392 could not be grasped in any of the predefined positioned. The control system 15 may place the system 10 in a standby state until a service intervention is performed.

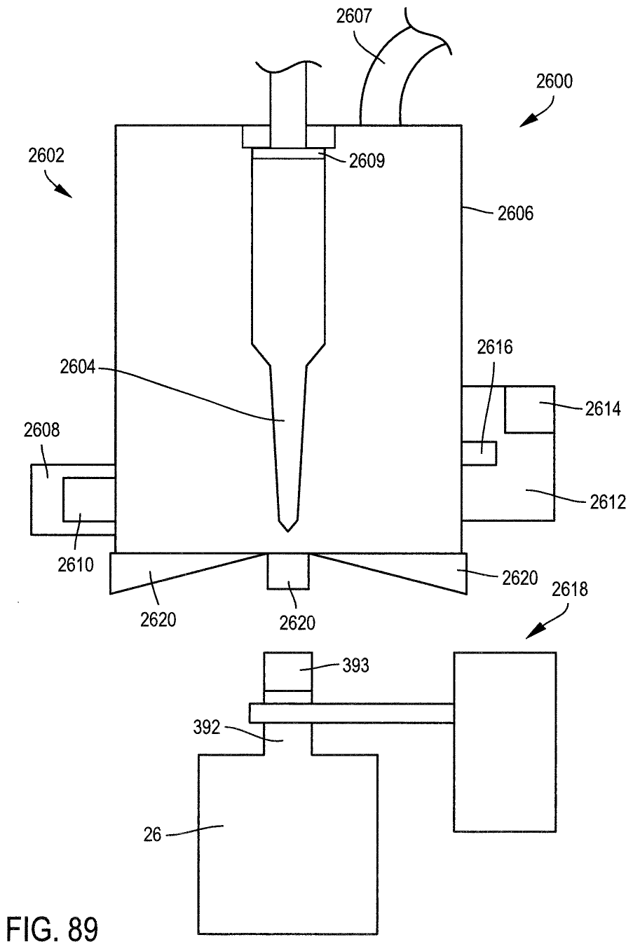

Referring now to FIG. 89, in certain system 10 embodiments, it may be desirable to perform filling of a bag 26 while limiting requirements for tight environmental control of a large area in which bags 26 are filled. In certain systems 10, an enclosure 12 which is controlled to a clean room standard may not be included. Alternatively, in certain systems 10 it may be desired that any enclosure 12 used be controlled to the least stringent clean room standard possible to limit complexity of a system 10. In such embodiments, bags 26, which have been previously sterilized, may be filled in a locally established or miniaturized clean room like environment. This environment or fill zone may only be present in a small space, for example, a chamber or partitioned region where dispensing of fluid into a bag 26 takes place. The fill zone may also be in open communication with other less stringently controlled regions zones. A fill zone may be sized to accept a portion of a bag 26, for example, a port 392 or end portion of a port 392. In some embodiments, the fill zone may have a volume of not more than 100 ml (e.g. 75 ml or less). In other examples, the fill zone may have a volume of not more than 200 ml (e.g., 125 ml or less). The fill zone volume may for example conform to or exceed particle requirements, as well as appropriate relative pressure, air exchange rate, and/or air velocity set points for a selected cleanroom level (e.g. ISO 5 clean room set points). The fill zone may also be arranged to support substantially unidirectional or laminar airflow.

Still referring to FIG. 89, an example fill station 2600 is depicted. The fill station 2600 may include a dispenser assembly 2602. In the example embodiment, the dispenser assembly 2602 includes a dispensing sharp 2604. The dispensing sharp 2604 may be or be placed in fluid communication with a fluid source. The fluid source may be an output of a mixing system which combines injection quality water with constituent to form a desired fluid for filling of a bag 26. Any systems described in relation to U.S. Pat. No. 11,980,587, issued May 14, 2024, and entitled Systems, Methods, and Apparatuses for Producing and Packaging Fluids (Attorney Docket No. 00101.00325.AA697) may be used. Alternatively, the fluid source may be a source of purified water or water conforming to a predefined set of criteria. In one such example, fluid may be generated from a distillation device (e.g. vapor compression distillation device) and potentially one or more additional water treatment apparatus (e.g. charcoal filter(s), ultrafilter(s), electrodeionizer, capacitive deionization assembly, etc. In other examples, fluid may be generated from at least one reverse osmosis assembly and potentially one or more additional water treatment apparatuses (e.g. any of those described above). Combinations of the above are also possible. A filter 2609 may be disposed intermediate the output of the fluid source and the dispensing sharp 2604. The filter 2609 may, for example, be a 0.2 micron filter. Where a distillation device is used, any such device described in U.S. Patent Publication No. US 2021-0395109, filed Mar. 29, 2021, and entitled Water Vapor Distillation Apparatus, Method, and System (Attorney Docket No. AA542) which is hereby incorporated by reference in its entirety may be used. Any of the fluid handling systems 3900 described herein (see, e.g., FIGS. 229A-C) may also be used.

The dispensing sharp 2604 may be disposed within a chamber or cartridge 2606 that surrounds the dispensing sharp 2604. Alternatively, in some embodiments, the dispensing sharp 2604 may be included as part of a replaceable cartridge 2606. Though a variety of exemplary cartridges 2606 are described herein, certain example cartridges 2606 may include a first end and a second end. In such examples, the first end may be an input end and may receive at least one fluid (e.g. clean air and/or a liquid for filling the bag 26). The second end may be a bag 26 interface end which may, for instance, accept a port 392 of a bag 26. The first end may be closed or open depending on the embodiment. The second end may be open to provide a receptacle for the portion (e.g. port 392) of the bag 26 through which the dispensing sharp 2604 gains access to the interior volume of the bag 26.

The dispensing sharp 2604 may be removably coupled to the cartridge 2606, form a part of a cartridge 2606, or may be coupled to a portion of the dispensing assembly 2602 which may be separable from the remainder of the dispensing assembly 2602. Thus, the dispensing assembly 2062 may include a durable portion and a disposable portion (e.g. cartridge 2606) or replaceable unit which is swapped out as needed. Other portions of the dispensing assembly 2602 may remain in place at the fill station 2600 as a cartridge 2606 is exchanged. This may allow for the dispensing sharp 2604 (and other cartridge 2606 components) to be replaced on some periodic schedule or in the event that the dispensing sharp 2604 dulls. Alternatively, the dispensing assembly 2602 may be a consumable which is periodically replaced as the fill station 2600 is used. In still other embodiments, the dispenser assembly 2602 may be a durable assembly and may be replaced in its entirety after a designated usage life has been met (see, e.g., FIGS. 141-142C).

The dispensing sharps 2604 described herein may be any suitable type of sharp. Preferably, the dispensing sharp 2604 may withstand repeated punctures of septa 393 without damage or significant dulling. Additionally, the dispensing sharp 2604 may preferably be chosen based on its ability to cleanly pierce a septum 393 without compromising the septum's 393 ability to reseal. Atraumatic needle varieties may for example be used for the dispensing sharp 2604. In certain example embodiments, the dispensing sharp 2604 may be a pencil tipped type sharp including at least one side port. In some embodiments, the dispensing sharp 2604 may be a Whitacre, Sprotte, Pencan, or Gertie Marx type needle with two side ports disposed in opposition to one another (see, e.g. FIG. 103A and FIG. 142C). Exemplary dispensing sharps 2604 may have a gauge of 16-18 and a length of less than one inch (e.g. 0.75 inches). Dispensing sharps 2604 may be constructed of a metallic material such as stainless steel.

A cartridge 2606 may be plumbed or otherwise coupled into communication with a source of clean air (e.g. HEPA or ULPA filtered air). A clean air supply feed 2607 is depicted coupled to the input end of the cartridge 2606 in the example embodiment depicted in FIG. 89. Clean air may pass through the cartridge 2606 from the input end to the open end to establish a positive pressure within the cartridge 2606 relative to the surrounding or downstream environment. Additionally, the rate of air flow through the cartridge 2606 may be sufficient to ensure a predefined rate of air exchanges within the cartridge 2606 per given unit of time. The air flow passing through the cartridge 2606 may be substantially laminar and may be preconditioned to increase its laminar character.

In some examples, the clean air may be directed through a volume in the immediate vicinity of the dispensing sharp 2604 and any hub 2640 (see, e.g., FIG. 103C) to which the dispensing sharp 2604 is coupled. The cartridge 2606 may include a wall or walls to define this volume and partition it off from the surrounding environment. Such wall(s) may be discrete components of example cartridges 2606 and may be installed into place when a cartridge 2606 is assembled. In such examples, certain components of the dispensing assembly 2602 may be fully outside of the clean air flow path. The clean air flow path through the cartridge 2606 or a portion thereof may define the fill zone for the fill station 2600.

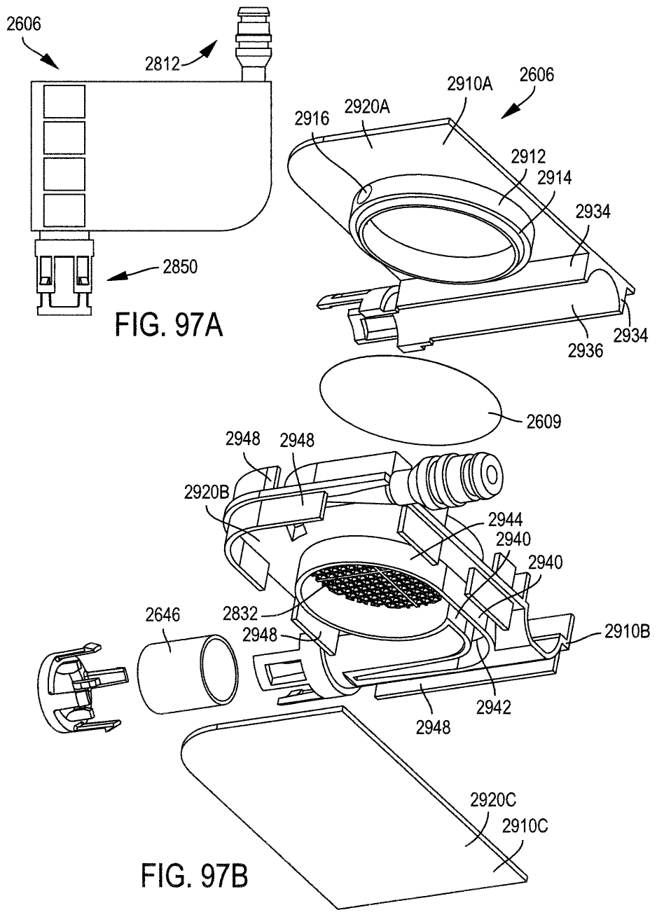

An example dispensing assembly 2602 may include an irradiation assembly 2608. At least a portion of the irradiation assembly 2608 may, for example, be disposed outside of a clean air flow path around the dispensing sharp 2604. Cartridges 2606 may be separable from irradiation assemblies 2608 such that the irradiation assembly 2608 is a reusable component of the fill station 2600 which does not need to be replaced when a cartridge 2606 is replaced. Example irradiation assemblies 2608 may include at least one antimicrobial light emitter 2610. The at least one antimicrobial light emitter 2610 may emit light in a portion of the spectrum or at a wavelength which is damaging to microbes. For example, an irradiation assembly 2608 may include a plurality of UV light emitters which emit light in the UVC range (e.g. at 265 nm+/−5 nm). The antimicrobial light emitter(s) 2610 may be any suitable light emitter, but in various embodiments may be LED light emitters. Where multiple light emitters 2610 are used, the light emitters 2610 may be spaced about the cartridge 2606 at, for example, even angular intervals (e.g. 120° or 90°). The light emitters 2610 may also be arrange to direct emitted light at a common point within the interior volume or fill zone defined within the cartridge 2606. The irradiation assembly 2608 may also include one or more heat sink and/or fan to assist in dissipation of heat generated by the irradiation assembly 2608 or individual light emitters 2610 of the irradiation assembly 2608. At least one temperature sensor may be included in the irradiation assembly 2608. For example, at least one temperature sensor (see, e.g. 2611 of FIG. 90) such as an RTD may be in association with each individual light emitter 2610. A control system 15 may monitor the temperature signal output from the temperature sensor(s). In the event that the temperature sensor(s) output a signal indicative of a temperature in excess of a threshold, the control system 15 may power off the light emitters 2610 and/or require a cool down period elapses before again commanding illumination of the light emitters 2610. In certain embodiments, the interior of the cartridge 2606 may be at least partially coated or covered with a reflective surface which reflects light emitted by the light emitter(s) 2610. In various embodiments, a UVC transparent or at least translucent barrier 2646 (see, e.g., FIG. 97B) may be disposed between each of the light emitters 2610 and the dispensing sharp 2604. Preferably, a material which is at least 75% transmissive to UVC ((e.g. 87% or over 85% at 265 nm) may be used. Fused quartz tubing may be used in some examples.

In some examples, and as shown in FIG. 89, an example dispensing assembly 2602 may also include one or more agent dispensing assembly 2612. An agent dispensing assembly 2612 may include an agent source reservoir 2614 which may be removably coupled to the agent dispensing assembly 2612 and replaced once depleted. The agent dispenser 2612 may include an output 2616 which may dispense agent from the agent source reservoir 2614 into the cartridge 2606. The output 2616 may be a sprayer, atomizer, or other ejector. In certain examples, the agent source 2614 may be an antimicrobial chemical agent. For example, the agent source 2614 may be a reservoir of 70% isopropyl alcohol.

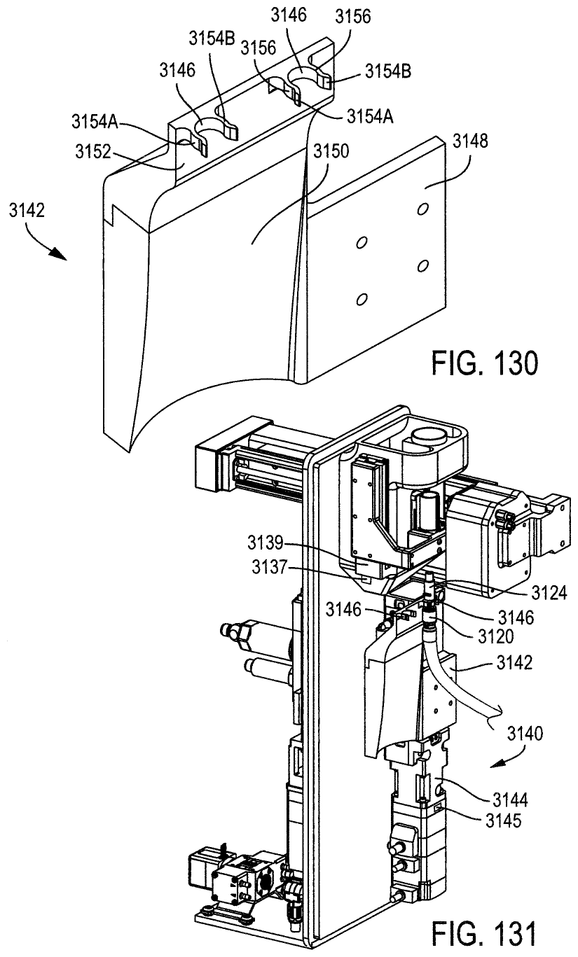

Still referring primarily to FIG. 89, sealed bags 26 including a port 392 with a septum 393 or internal membrane may be displaced to the fill station 2600 by a bag displacement assembly 2618. The bag displacement assembly 2618 may, for example, include a gantry, one or more linear displacement stage, or robotic arm with an electromechanical, pneumatic, or hydraulic gripper though any automated assembly may be used (see, e.g., upstream displacement assembly 4302 and bag retainer assembly 4306 of FIG. 68A). In some embodiments, the bag 26 may not be actively held by a gripper, but may instead be retained by a displaceable passive holder. Clean air may be provided to the cartridge 2606. The port 392 and septum 393 of the bag 26 may enter the cartridge 2606. The cartridge 2606 may be displaced to the port 392 or the bag 26 may be displaced toward the cartridge 2606 with a displacement stage (see, e.g. fill displacement stage 3142 of FIG. 130). At least one guide 2620 may be provided on the cartridge 2606 to help direct a port 392 into the cartridge 2606 and help align a port 392 with the dispensing sharp 2604. A control system 15 may activate the irradiation assembly 2608 after commanding relevant actuators to displace the port 392 into place. The irradiation assembly 2608 may be active for a predetermined period of time such that any exposed surfaces of the septum 393 receive at least a prescribed dose of irradiation (e.g. 180-190 mJ/cm²). The prescribed dose of irradiation may differ from embodiment to embodiment though the dosage may be selected to achieve an at least 6 log reduction of any microbial presence on the septum 393 surface. The dispensing sharp 2604 may also be in the illumination field of the irradiation assembly 2608 and may receive its own prescribed dosage of irradiation as this occurs. The dispensing sharp 2604 may puncture the septum 393 (e.g. by the port 392 being driving into the dispensing sharp 2604) such that an outlet opening in the dispensing sharp 2604 is in communication with the interior volume of the bag 26. Fluid may then be delivered through the dispensing sharp 2604 into the bag 26 to fill the bag 26. Where included, an agent dispenser 2612 may also direct agent onto the port 392 and/or septum 393 prior to puncturing the septum 393.

Where description herein refers to displacement, positioning, piercing, etc. of a port 392 or septum 393, it should be understood that the port 392 or septum 393 may be substituted for an access to any of a variety of reservoirs. Vials, cartridges, cassettes, partially flexible reservoirs, collapsible reservoirs, variable volume reservoirs, or any other fluid containing reservoir may be filled at fill stations 2600 described herein.

Referring now to FIG. 90, an example dispensing assembly 2602 embodiment is schematically depicted. As shown, the dispensing assembly 2602 may include a cartridge 2606. The cartridge 2606 may include an air flow channel 2802 which may extend from a first side of the cartridge 2606 to an opposing second side of the cartridge 2606. The air flow channel 2802 may form a generally straight-line path through the cartridge 2606 and may have a round, and in the example shown, substantially circular cross-section. As shown in FIG. 90, the air flow channel 2802 may be partially formed by a barrier 2646 such as a section of fused quartz tubing. The barrier 2646 may be captured within the cartridge 2606 as the cartridge 2606 is assembled. The air flow channel 2802 may receive clean air from a supply such as an air handling assembly 6200 which introduces filtered, laminarized air into an enclosure 12 of the system 10. As indicated by arrows 2804, air flow displacing through the air flow channel 2802 may substantially maintain is laminar character as it transits the cartridge 2606. The dispensing sharp 2604 and a portion of a hub 2640 to which the dispensing sharp 2604 is coupled may be disposed within the air flow channel 2802. Preferably, the hub 2640 is streamlined in shape and presents a minimal profile to limit impact on the laminar flow of air through the air flow channel 2802.

The example cartridge 2606 may also receive fluid from a fluid source 2810 (e.g., injection quality water or medical fluid mixed by the system 10). As shown the cartridge 2606 may include a connection port 2812. The connection port 2812 may include a series of sealing members 2814A, B which are in spaced relation to one another about the exterior surface of the connection port 2812. In the example, the connection port 2812 has sections of different diameters with the smallest diameter section being at the end of the connection port 2812. A sealing member 2814A, B is included on each section. Additionally, a terminal end of the connection port 2812 may be covered by a frangible 2816.

To establish fluid communication with the fluid source 2810, the connection port 2812 may be introduced into a receptacle 2818 of a supply manifold 2820. A spike 2822 in the receptacle 2818 may interrupt the frangible 2816. The spike 2822 may be in fluid communication with the fluid source 2810. Fluid may flow from the fluid source 2810 through the spike 2822 and connection port 2812 and through the cartridge 2606 via one or more cartridge 2606 flow paths 2830. The supply manifold 2820 may be coupled to a manifold displacement assembly 2821 which may be actuated to displace the supply manifold 2820 relative to the connection port 2812.

In certain embodiments, the connection port 2812 may be displaced into a disinfection position within the receptacle 2818 before the frangible 2816 is contacted or compromised with the spike 2822. In the disinfect position, the connection port 2812 may be partially installed in the receptacle 2618 such that a sealing member 2814B most distal the frangible 2816 establishes a fluid seal with the sidewall of the receptacle 2818. This fluid seal may be formed intermediate the open end of the receptacle 2818 and a disinfection outlet path 2824 extending from the receptacle 2818 (e.g. to a drain or other discard destination 2826). Disinfecting fluid may be dispensed through the spike 2822 and into the receptacle 2818. The disinfecting fluid may be water which has been heated to a disinfection temperature set point or range. In certain examples, injection quality water from the fluid source 2810 may be heated to 60° C. or higher and fed to the receptacle 2818. As the sealing member 2814B blocks the fluid from exiting the receptacle 2818, the fluid may flow into the disinfection outlet path 2824. As this occurs, the terminal end of the connection port 2812 up to the sealing member 2814B may be exposed to the disinfection fluid. As shown, the receptacle 2818 includes different diameter portions such that when the connection port 2812 is in the partially installed position only sealing member 2814B is in contact with the receptacle wall 2818. This ensures disinfecting fluid may reach the disinfection outlet channel 2824 and also bathes the connection port 2812 up to the sealing member 2814B on the larger diameter portion of the connection port 2812. Disinfection fluid may be supplied through the receptacle 2818 for a preset period of time. The period of time may be selected based on the disinfection fluid and the temperature of the disinfection fluid. Once the disinfection cycle has completed, the connection port 2812 may be advanced to a fully installed position in the receptacle 2818. In the fully installed position, the frangible 2816 may be interrupted by the spike 2822 and the fluid source 2810 may be in fluid communication with a dispensing sharp 2604 via the cartridge flow path(s) 2630. The sealing member 2814A most proximal the end of the connection port 2812 may establish a seal against the receptacle 2818 as the connection port 2812 is brought to the fully installed position.

In various examples, at least one filter 2609 may be included in the cartridge 2606 and may be disposed in an intermediate point in the flow path through the cartridge 2606. The filter 2609 may provide redundant purification for fluid provided from the fluid source 2810. The filter 2609 may also provide greater latitude in choice of frangible 2816 as the filter 2609 may serve to catch any particulate formed as the connection port 2812 is spiked. The filter 2609 may be any suitable filter. In certain examples, the filter 2609 may be a filter membrane with a pore size down to 0.2 micron. In examples where a filter membrane is used, a filter support 2832 may also be included in the form of a grate against which the filter 2609 may be retained. The filter support 2832 may be on the downstream side of the filter 2609. The filter 2609 may be included in a filter assembly 2838 (see, e.g., FIG. 93) disposed within the cartridge 2606. The filter assembly 2838 may, in certain examples, be assembled separately and placed into the cartridge 2606.

As with other embodiments described herein, the dispensing assembly 2602 may also include an irradiation assembly 2608 with one or more light emitters 2610. An irradiation assembly 2608 with an aperture 2630 for accepting a cartridge 2606 may, for example, be used. A dock 2624 or other arrangement for retaining the cartridge 2606 may be included in the dispensing assembly 2602.

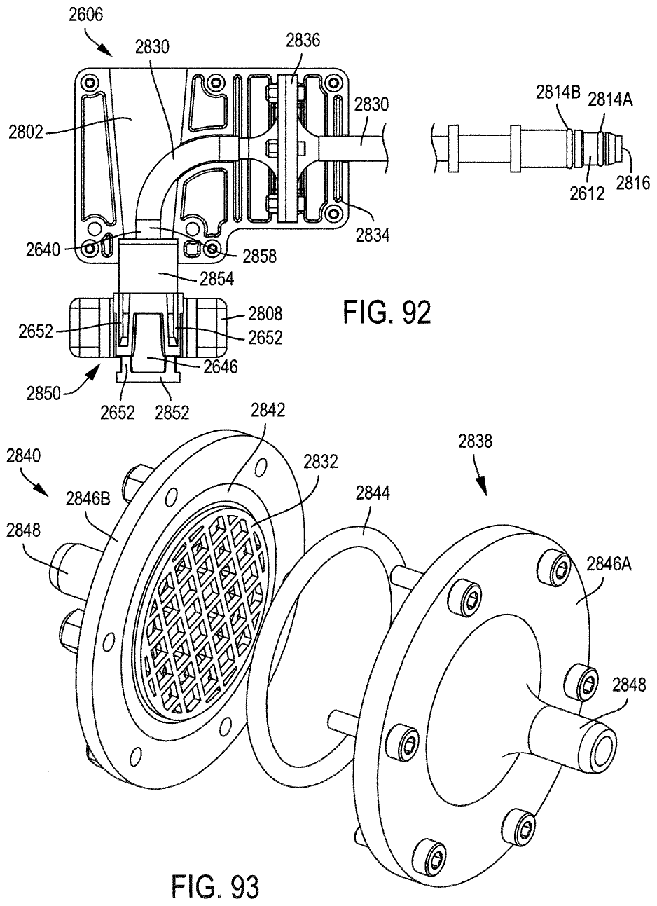

Referring now primarily to FIGS. 91-92, an example embodiment of the cartridge 2606 described in relation to FIG. 90 is depicted. An irradiation assembly 2608 is also depicted. As shown, the cartridge 2606 may include a connector port 2812 having a set of sealing members 2814A, B and a frangible 2816. The connector port 2812 may be disinfected and placed into fluid communication with a fluid source 2810 as described in relation to FIG. 90. The cartridge 2606 may include a housing assembly 2834 with a fluid flow path extending therethrough. A portion of the housing assembly 2834 is removed in FIG. 92 to illustrate the interior portions of the cartridge 2606. A bay 2836 for receiving a filter assembly 2838 may be defined in the housing assembly 2834. The filter assembly 2838 may be disposed at an intermediate point in the flow path through the cartridge 2606. Fluid from the fluid source 2810 may flow through a cartridge flow path 2830 to an inlet side of the filter assembly 2838 and through the filter 2609. Fluid may exit the filter assembly 2838 through an outlet side of the filter assembly 2838 and transit through another cartridge flow path 2830 to the dispensing sharp 2604.

Referring primarily to FIG. 93, a partially exploded view of an example filter assembly 2838 is depicted. The filter 2609 is hidden in FIG. 93, but could be any desired membrane filter. As shown, the filter assembly 2838 includes a filter support 2832 which in the example embodiment is a grated disk. The filter 2609 may be placed against the upstream side of any filter support 2832 such that the filter support 2832 buttresses the filter 2609 against the pressure of the fluid flowing through the cartridge 2606. The filter assembly 2838 also includes a filter housing 2840 having an inlet section 2846A and an outlet section 2846B. Each of the inlet and outlet sections 2846A, B may include a fitting 2848 for attachment of a cartridge fluid flow path 2830. The filter housing 2840 may include a seat for the filter support 2832 which is surrounded by a trough 2842. The trough 2842 may accept a sealing member 2844 such as an o-ring. The filter 2609 may be placed against the filter support 2832 and overhang the sealing member 2844. When the inlet and outlet section 2846A, B of the filter housing 2840 are coupled to one another, the filter 2609 may be captured within the filter housing 2840 and a seal surrounding the portion of the filter 2609 adjacent the filter support 2832 may be established. Thus, fluid entering the filter assembly 2838 may only pass to the outlet of the filter assembly 2838 through the filter 2609. Though fasteners are shown in the example embodiment, the inlet and outlet section 2846A, B of the filter housing 2840 may be coupled to one another in any suitable manner (e.g. sonic welding, laser weld, etc.).

Referring again to FIGS. 91-92, a barrier retention assembly 2850 is included as part of the housing assembly 2834 of the example cartridge 2606. The barrier retention assembly 2850 is coupled into the portion of the air flow channel 2802 defined in the remainder of the housing assembly 2834. The barrier retention assembly 2850 may retain a barrier 2646 (e.g. fused quartz tube segment) through which an irradiation assembly 2608 may irradiate the dispensing sharp 2604 and a septum 393 advanced into the cartridge 2606. In the example embodiment, the barrier 2646 is retained by first and second bodies 2852, 2854 with cantilevered members 2652. The cantilevered members 2652 may be engaged with one another to capture the barrier 2646. The cantilevered members 2652 may be positioned so as to not offer obstruction or minimally obstruct the illumination path from the light emitters 2610 to the interior of the barrier 2646.

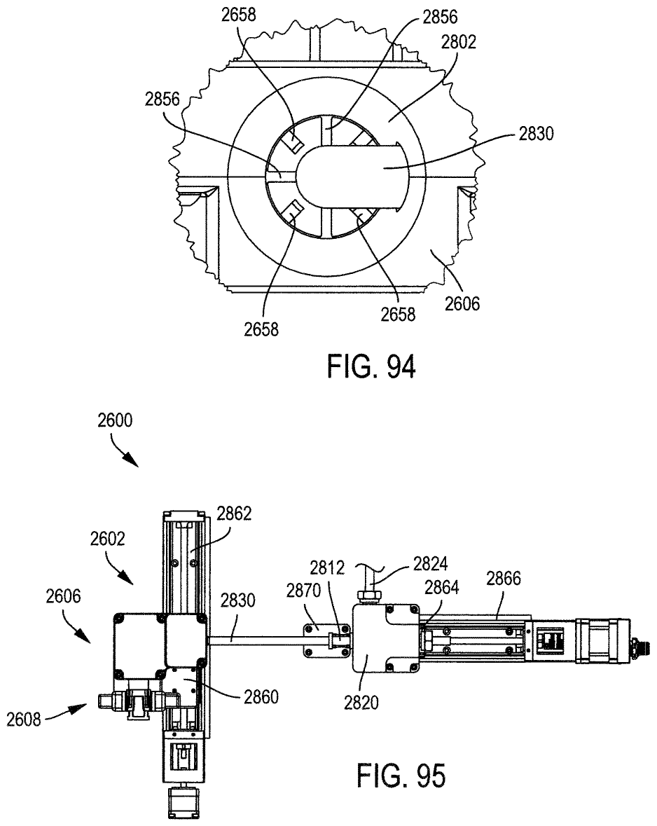

Referring now also to FIG. 94, a detailed top plan view of a portion of the example cartridge 2606 of FIGS. 91-92 is depicted. As shown, the air flow channel 2802 has a cross section which tapers from a largest area to a smaller area as distance from the input end of the cartridge 2606 increases. The hub 2640 for the dispensing sharp 2604 is suspended by a number of cross pieces 2856 in line with the axis of the air flow channel 2802. A cartridge flow path 2830 is shown extending into communication with a fitting 2858 (see, e.g., FIG. 92) of the hub 2640. Guides 2658, which may be included on the first body 2852 are also visible in FIG. 94. The guides 2658 may assist in directing a port 392 into position along the axis of the air flow channel 2802 as the port 392 is brought into proximity of the dispensing sharp 2604.

Referring now to FIG. 95, an example filling station 2600 is depicted including the dispensing assembly 2602 of FIGS. 91-92. As shown, the cartridge 2606 and irradiation assembly 2608 may be coupled to a stage 2860 of a filling actuator 2862. The filling actuator 2862 may be driven via commands from the control system 15 to displace the cartridge 2606 and irradiation assembly 2608. Additionally, the supply manifold 2820 may also be placed on a stage 2864 of a connection actuator 2866. The connection actuator 2866 may be driven via commands from the control system 15 to displace the supply manifold 2820 relative to the connection port 2812. The filling station 2600 may also include a retainer 2870 to which the connection port 2812 may be coupled in order to render the connection port 2812 stationary as it introduced and spiked into the receptacle 2818 of the supply manifold 2820.

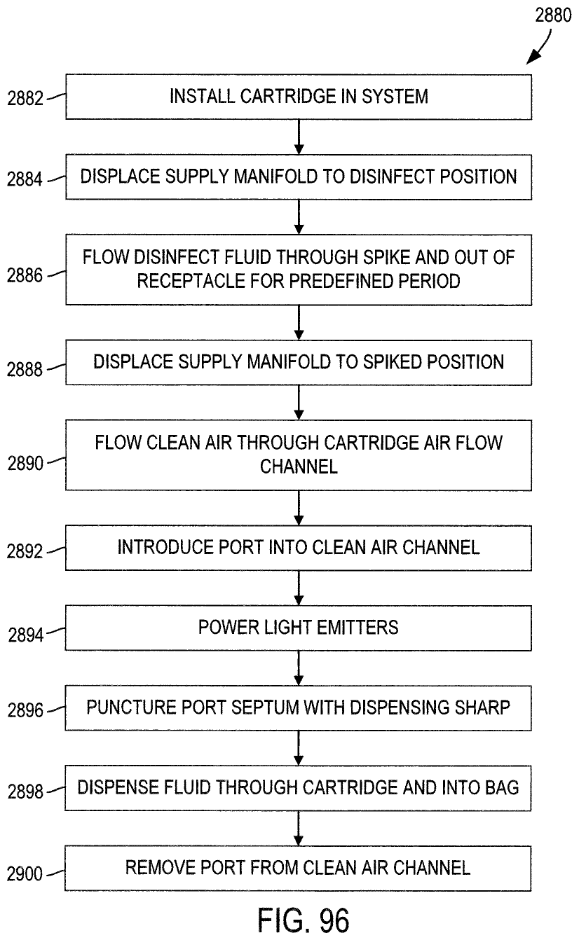

Referring now also to FIG. 96, a flowchart 2880 detailing a number of example actions which may be executed to fill a bag 26 at a fill station 2600 is depicted. As shown, in block 2882, a cartridge 2606 may be installed in a fill station 2600. In block 2884, a supply manifold 2820 may be displaced into a disinfection position. As described above in relation to FIG. 90, in this position a connection port 2812 of the cartridge 2606 may be partially inserted into a receptacle 2818 of the supply manifold 2812. Disinfecting fluid (e.g. high temperature water) may flow through the spike 2822 of the receptacle 2818 and out of an outlet 2824 of the receptacle 2818 for a predefined period of time in block 2886. The supply manifold may be displaced, in block 2888, to a spiked position. As this occurs, a spike 2822 of the receptacle 2818 may puncture through a frangible 2816 on the connection port 2812 and establish a fluid communication pathway with the dispensing needle 2804 though the cartridge 2606. In block 2890, clean air may flow through the air flow channel 2802 in the cartridge 2606. A port 392 of a bag 26 may be introduced into the air flow channel 2802 in block 2892. To accomplish this, the filling actuator 2862 may be commanded to displace the cartridge 2606 and irradiation assembly 2608 to position in which a port 392 of a bag 26 is disposed within the portion of the air flow channel 2802 formed by the barrier 2646. Alternatively, the bag 26 could be retained on an actuator stage which is displaced to drive the port 392 into the air flow channel 2802. Once the port 392 is in a ready position, the irradiation assembly 2608 may be powered such that the light emitters 2610 irradiate the septum 393 in block 2894. The septum 393 may be punctured by the dispensing sharp 2804 in block 2896. In block 2898, fluid may be dispensed into the bag 26 via the dispensing sharp 2604. The port 392 may be removed from the air flow channel 2802 in block 2900. This may be accomplished by commanding an actuator to lower a stage on which the bag 26 is retained with a control system 15. Alternatively, the control system 15 may govern operation of a filling actuator 2862 to displace the irradiation assembly 2608 and cartridge 2606 away from the port 392.

Referring now to FIG. 97A-B, another exemplary embodiment of a cartridge 2606 which may be installed in a dispensing assembly 2602 is depicted. The cartridge 2606 includes a stack of plates 2910A-C. The plates 2910A-C may be coupled together in any suitable fashion (e.g. sonic welding, laser welding, adhesive, solvent bonding, etc.). Where laser welding is used, the second plate 2910B may be formed of a material which absorbs the wavelength of light emitted by the welding laser (e.g. black polymer). The first and third plates 2910A, C may be constructed of material which is substantially translucent to the wavelength (e.g. various clear polymers). Each of the plates 2910A-C may include a main body 2920A-C which may include one or more raised or recessed features. In some examples, one of the plates 2910C may be a substantially flat member devoid of raised or recessed features. When the plates 2910A-C are coupled together, the raised and recessed features of the plates 2910A-C may cooperate to form the cartridge flow paths 2630, portions of the filter assembly 2838, and the air flow channel 2802. A portion of the barrier retention assembly 2850 may also be formed by the plates 2910A-C.

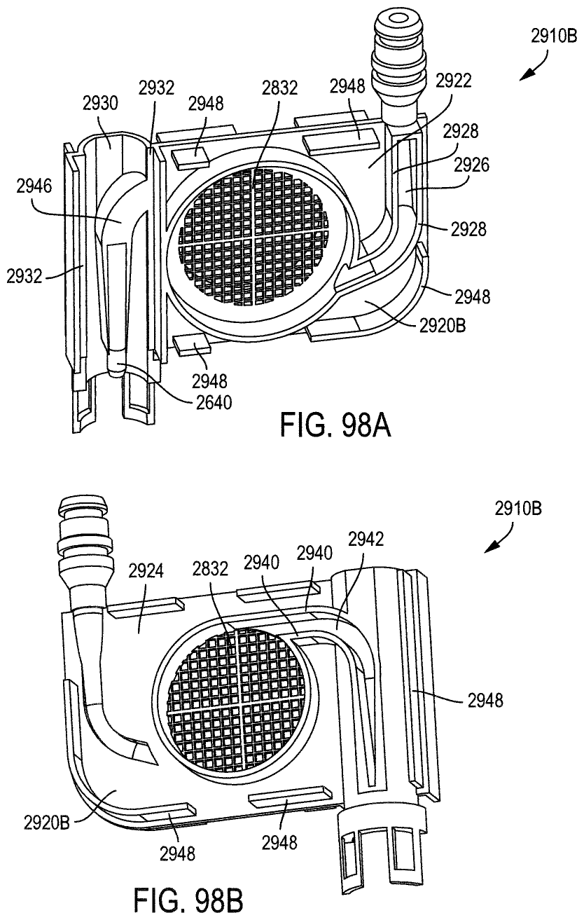

Referring now primarily to FIG. 97B in conjunction with FIGS. 98A-B, the first plate 2910A may include a filter capture ridge 2912. The filter capture ridge 2912 in the example embodiment is in the shape of a ring. The face of the filter capture ridge 2912 most distal to the main body 2920A of the first plate 2910A may include a sealing member seat 2914 which may accept a sealing member (e.g. o-ring). A filter membrane 2609 may be captured against the filter support 2832 when the first plate 2910A is coupled to the second plate 2910B or mid plate. The filter support 2832 may be a grated region of the main body 2920B of the second plate 2910B. The sealing member may also be compressed between the second plate 2910B and the filter capture ridge 2912 when the first and second plates 2910A, B are coupled together.

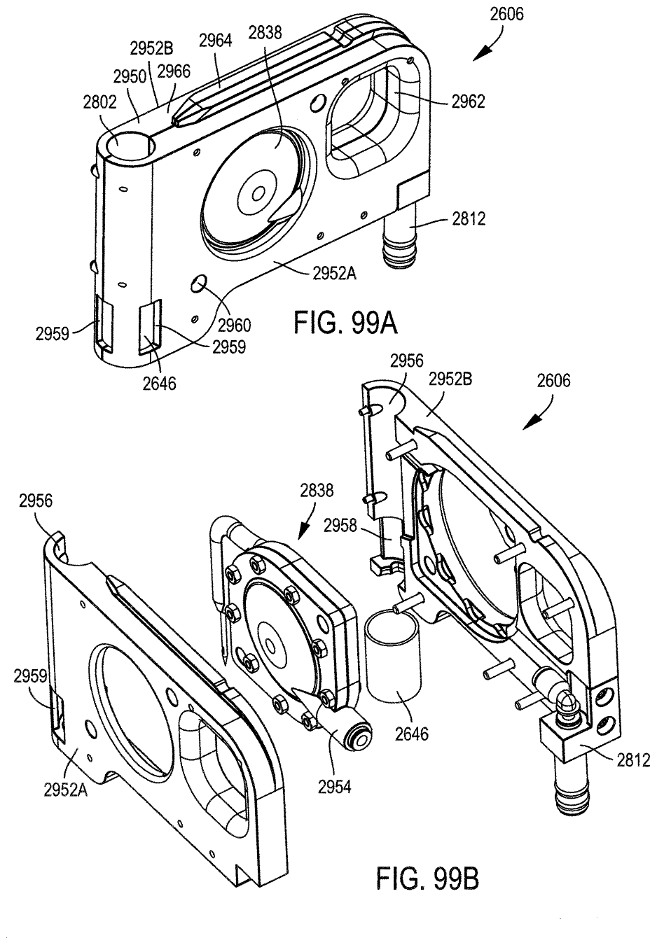

The second plate 2910B may form a midbody of the cartridge 2606 and include an inlet face 2922 (best shown in FIG. 98A) and an outlet face 2924 (best shown in FIG. 98B). The inlet face 2922 may include an inlet flow channel 2926 defined by a set of inlet walls 2928. The inlet flow channel 2926 may extend from the connection port 2812 to the filter assembly 2838. To facilitate ease of manufacture, the connection port 2812 may be a separate assembly which may couple to the cartridge 2606 (see, e.g., FIG. 99B). This may allow each of the plates 2910A-C to be injection molded. When the first plate 2910A is coupled to the second plate 2910B, the first plate 2910A may be coupled to the face of the inlet walls 2928 most distal the main body 2920B of the second plate 2910B. Thus, a sealed fluid conduit within the cartridge 2606 may be formed placing the connection port 2812 in fluid communication with the filter assembly 2938. As best shown in FIG. 97B, the filter capture ridge 2912 may include a passage 2916 therethrough which aligns with the inlet flow channel 2926. Thus, the filter capture ridge 2912 may not block flow to the filter assembly 2838 from the inlet flow channel 2926.

The inlet face 2922 may also include a recessed trough 2930. The recessed trough 2930 may form a section of the air flow channel 2802 through the cartridge 2606. As shown, the trough 2930 may be flanked by shelf regions 2932. The first plate 2910A may include a set of raised walls 2934 with a trench 2936 therebetween. In the exemplary embodiment depicted in FIG. 97B, the trench 2936 is a half-circular shape. When the second plate 2910B is coupled to the first plate 2910A, the raised walls 2934 may seat against the shelf regions 2932. In some embodiments, the raised walls 2934 may be coupled to the shelf regions 2932 (e.g. via laser or sonic weld). The trough 2930 and trench 2936 may cooperate to form the air flow channel 2802 of the cartridge 2606. In some examples, the airflow channel 2802 may have a substantially constant cross-sectional area or may have a varying cross-sectional area over its length (see, e.g., FIG. 90). Each of the first plate 2910A and the second plate 2910B include a portion of the barrier retention assembly 2850 in the example embodiment. When the first and second plates 2910A, B are coupled together, a first body 2852 of the barrier retention assembly 2850 may be formed by these plates 2910A, B. A second body 2854 may be coupled to the first body 2852 (e.g. as described in relation to FIGS. 91-92) to capture the barrier 2646 in place within the cartridge 2606.

The outlet face 2924 of the second plate 2910B may include a set of outlet walls 2940 which define an outlet flow path 2942. The outlet flow path 2942 may extend from the filter assembly 2838 to the dispensing sharp 2604. The filter support 2832 may also be surrounded by a wall 2944 which, in the example, is continuous with the outlet walls 2940. When the third plate 2910C is coupled to the second plate 2910B, the third plate 2910C may be coupled to the face of the outlet walls 2940 most distal the main body 2920B of the second plate 2910B. Thus, a sealed fluid conduit within the cartridge 2606 may be formed from the connection port 2812, through the filter assembly 2938, and to the dispensing sharp 2904. Wall 2944 may be similarly coupled to the third plate 2910C to seal the volume surrounding the filter support 2932. As shown, a relief region 2946 may be present in the trough 2930 on the inlet face 2922 to afford greater depth to the outlet flow path 2942. The relief region 2946 may also form the hub 2640 to which dispensing sharp 2604 (see, e.g., FIG. 103C) is coupled.

Preferably, and as shown in FIGS. 97A-98B, any outlet flow path 2942 leading from a filter assembly 2838 in a cartridge 2606 may be positioned at or near a top portion of the filter assembly 2838. Filter membranes 2609 tend to resist passage of gas when wetted. This position of the outlet flow path 2942 may facilitate priming of the cartridge 2606 with liquid as gas in the filter assembly 2838 may rise as the priming process transpires. Trapping of air or gas on the upstream side of the filter 2609 may thus be inhibited. In turn, this may help to ensure consistent flow rates through filter assemblies 2838 in each cartridge 2606 installed in a system 10. Flow rates used during priming of any cartridges 2606 described herein may be selected to be above a minimum priming flow threshold. This threshold may be empirically determined and may be selected to ensure that gas is suitably primed out of the upstream side of the filter assembly 2832 prior to a point where the filter membrane 2609 becomes fully wetted by wicking of priming fluid.

Each of the inlet face 2922 and outlet face 2924 of the second plate 2910B may include peripheral ribs 2948 at the periphery of the second plate 2910B. The peripheral ribs 2948 may provide additional material which the first and third plates 2910A, C may be coupled to when the cartridge 2606 is assembled.

Referring now to FIGS. 99A-B, another example cartridge 2606 is depicted. As shown, the cartridge 2606 may include a housing 2950 formed by a first and second body 2952A, B. The first and second body 2952A, B may be coupled to one another and capture a filter assembly 2838 therebetween. The first and second body 2952A, B may be coupled to one another in any suitable manner. Though fasteners are depicted, the first and second body 2952A, B could be welded (e.g. laser, ultrasonic), adhered, solvent bonded, etc. to one another to form the housing 2950. The housing 2950 may include a set of guides 2960. The guides 2960 in the example embodiment are apertures which extend through the cartridge 2606. The guides 2960 may accept pins or projections on a mounting or docking assembly of the system 10. The housing 2950 also includes a grip passage 2962 which may provide a grasp point for a user when loading and unloading a cartridge 2606 into a system 10. A peripheral rib 2964 may also be included on at least one sidewall 2966 of the housing 2950. The peripheral rib 2964 may, for example, bestow asymmetry to the cartridge 2606 such that it may only fit into a receiver of the system 10 in a prescribed orientation.

Still referring to FIGS. 99A-B, a connection port 2812 may be coupled to the housing 2950. In the example embodiment, the connection port 2812 is coupled to the second body 2952B of the housing 2950. A cartridge flow path 2830 (e.g. tubing, not shown) may extend from the connection port 2812 to an inlet port 2954 of the filter assembly 2838 to place the connection port 2812 into fluid communication with the filter assembly 2838. The dispensing sharp 2604 and hub 2640 may be formed as part of the filter assembly 2838.

Each of the first and second body 2952A, B may include an air flow channel section 2956. The air flow channel sections 2956 may each have a recessed region 2958. A number of fenestrations 2959 which extend through the first and second body 2952A, B may be present at the recessed region 2958. A barrier 2646 (e.g. a tube segment formed of fused quartz) may be captured within the recessed regions 2958 when the housing 2950 is assembled. Thus, the first and second body 2952A, B and barrier 2646 may cooperate to form the air flow channel 2802 through the cartridge 2606. The fenestrations 2959 may align with the positions of light emitters 2610 of an irradiation assembly 2608 when the cartridge 2606 is in place within a system 10.

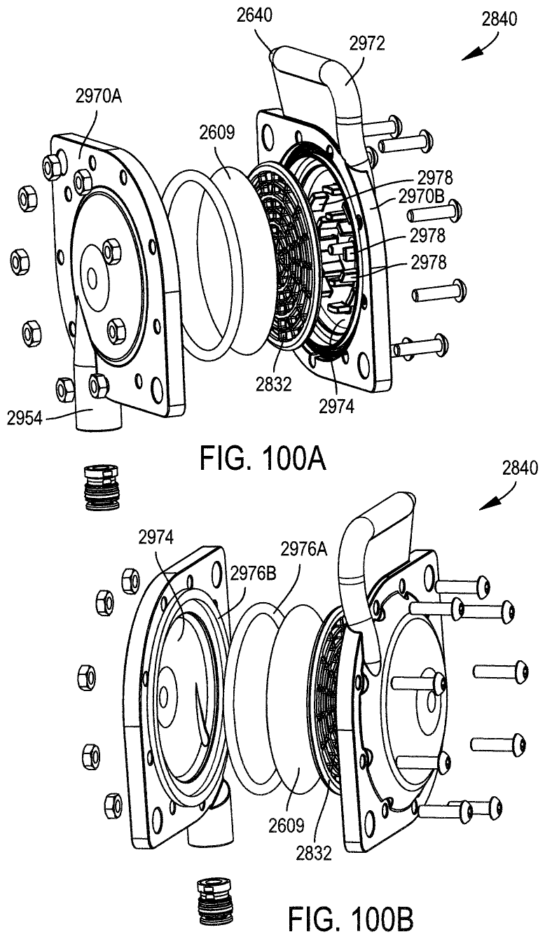

Referring now to FIGS. 100A-B, exploded views of the example filter assembly 2838 of FIG. 99B are depicted. Example filter assemblies 2838 may include a filter housing 2968 (see, e.g., FIG. 99B). The filter housing 2968 may be constructed of a first and second filter housing body 2970A, B. The filter housing bodies 2970A, B may be coupled together in any suitable manner (e.g. laser welding, ultrasonic welding, adhesive, solvent bonding, fasteners, etc.). One of the filter housing bodies 2970A, B may define a filter assembly inlet 2954. Alternatively, the filter assembly inlet 2954 may be formed when the filter housing bodies 2970A, B are coupled together and each of the filter housing bodies 2970A, B may include a portion of the filter assembly inlet 2954. One of the filter housing bodies 2970A, B may define a filter assembly outlet 2972. Alternatively, the filter housing bodies 2970A, B may cooperate to form the filter assembly outlet 2972 when coupled together. In some embodiments, and as shown, the filter assembly outlet 2972 may form the hub 2640 to which the dispensing sharp 2604 (see, e.g., FIG. 99B) is coupled. The internal face of each filter housing body 2970A, B may include a central depression 2974.

When the filter housing bodies 2970A, B are coupled together, a filter 2609 may be captured within the filter assembly 2838. One or more sealing members 2976A, B may become compressed against the filter 2609 to inhibit any flow around the filter 2609. Additionally, one or more sealing members 2976A, B may assist in preventing leaks from the filter assembly 2838 when the filter assembly 2838 is operating under high pressure. A filter support 2832 may also be retained within the filter assembly 2838 when the filter housing bodies 2970A, B are coupled. The central depression 2972 in the filter housing body 2970B on the permeate side of the filter 2609 may include a set of projections 2978 which may provide a rest for the filter support 2832 and inhibit distortion of the filter support 2932 under pressure.

Referring now to FIGS. 101A-103C, another example embodiment of a cartridge 2606 is depicted. The cartridge 2606 includes a cartridge housing 2950 formed from a first and second body 2952A, B. Each of the first and second body 2952A, B form a portion of the filter assembly 2838. In the example, the first and second body 2852A, B both include a depression 2982. The depression 2982 in the second body 2952B includes a number of projections 2978. When the first and second body 2952A, B are coupled together, the filter support 2832 may rest on the projections 2978 and the filter 2609 may be captured within the filter assembly 2838. One or more sealing member 2980 may become compressed inhibiting flow around the filter 2609 and leaks from the filter assembly 2838. Fluid may flow into the cartridge 2606 through a connection port 2812 and inlet flow path 2984 (see, e.g., FIG. 103A) which communicates with the depression 2972 in the first body 2952A. Permeate through the filter 2609 may pass to the dispensing sharp 2604 through an outlet flow path 2986 (see, e.g., FIG. 103B) extending from the depression 2972 in the second body 2952B.

In some examples, each of the depressions 2982 may include projections 2978. Two filter supports 2823 may be included and the filter 2609 may be sandwiched between the filter supports 2823. In some embodiments, this may facilitate withdrawal of fluid from the cartridge 2606. For example, a small volume of fluid may be withdrawn from the cartridge 2606 after each reservoir is filled. This may help to inhibit dripping of fluid from the dispensing sharp 2604 during use.

In some embodiments, the filter 2609 may be compromised when fluid is displaced in a direction other than the dispensing direction. The filter 2609 may be arranged to tear or pull out of the seal formed by the sealing member 2980 when fluid is withdrawn from the cartridge 2606 (at least when pressure in excess of a predefined magnitude is applied). The fluid handling system 3900 may only withdraw fluid from the cartridge 2606 when the cartridge 2606 is to be exchanged (e.g. usage life has been met). Flow and pressure characteristics detected by appropriate sensors in the fluid handling system 3900 may be monitored to check for a compromised filter 2609. Thus, the control system 15 may check sensor data from the fluid handling system 3900 to confirm that the cartridge 2606 is new and has not previously been used. The integrity of the filter 2609 may also be confirmed by ensuring sensor data is within predetermined ranges.

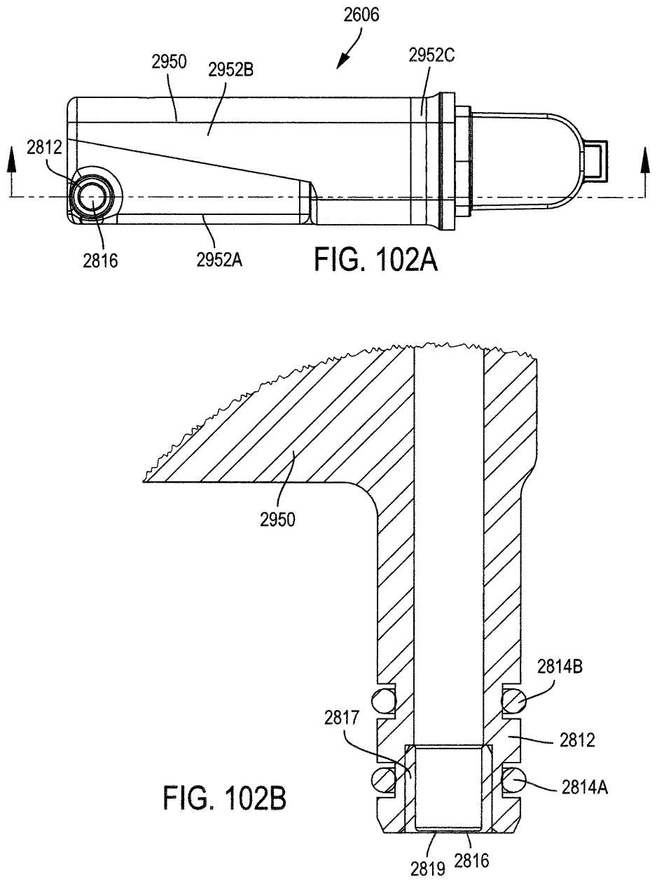

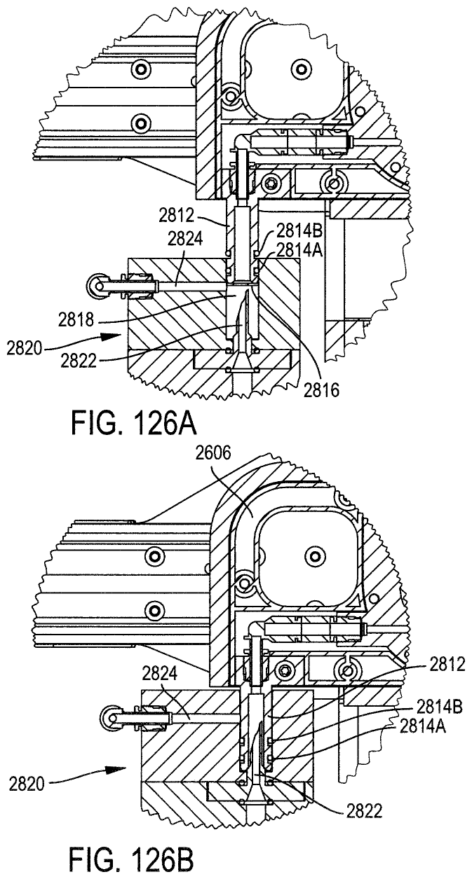

Referring primarily to FIGS. 102A-B, the connection port 2812 may be sealed with a frangible 2816. The frangible 2816 may be a plug with an elongate body 2817 which is closed on one end by an end wall 2819. The end wall 2819 may be relatively thin (e.g. 1-5 tenths of a millimeter thick). This end wall 2819 may be easily punctured by a spike 2822 (see, e.g. FIG. 126A) in a receptacle 2818 of a supply manifold 2820, but robust against inadvertent disruption. The frangible 2816 may be formed of an injection molded plastic material as a monolithic body. In some embodiments, a plastic such as polyphenylsulfone (PPSU) may be used. The frangible 2816 may be retained in the connection port 2812 via a friction fit though adhesive, solvent bonding or other manners of coupling may be used in alternative embodiments. Such frangibles 2816 may be particularly desirable as they do not include aluminum or other metals which it would be undesirable to have in communication with the fluid flow path. Additionally, a PPSU frangible 2618 would be compatible with exposure to high temperature fluid which may be used during disinfection of the connection port 2812.

The example cartridge 2606 in FIGS. 101A-103C further includes a third body 2952C. The third body 2952C may include a section of the air flow channel 2802. A retainer 2990 may be coupled to the third body 2952C and may capture a barrier 2646 between the retainer 2990 and the end of the air flow channel 2802 defined in the third body 2952C. The retainer 2990 may include guides 2658 which may help to position a port 392 of a bag 26 when the port 392 is introduced into the air flow channel 2802. When the retainer 2990 and barrier 2646 are in place, they may complete the air flow channel 2802 through the cartridge 2606. This may be desirable as this may remove seams running along the axial dimension of the air flow channel 2802.

As best shown in FIGS. 103B-C, the outlet flow path 2986 may extend to a hub 2640 which may protrude into the air flow channel 2802. A sealing member 2988 may be present at the transition of the outlet flow path 2986 from the second body 2952B to the third body 2952 and may be compressed when cartridge 2606 is assembled. The dispensing sharp 2604 may be coupled to the hub 2640.

Cartridges 2606 described herein may include a cover 2992 which may be coupled in place over the air flow channel 2802 of the cartridge 2606. The cover 2992 may seal against a sealing member 2994 (e.g. compliant gasket or overmolded material) on an exterior face of the housing 2950. In the example embodiment, the cover 2992 seats against a sealing member 2994 on the third body 2952C of the cartridge 2606. The third body 2952C includes a central projecting region 2953 in which the section of the air flow channel 2802 is defined. The third body 2952C also includes a perimeter region 2955 surrounding the central region 2953. The sealing member 2994 may be coupled to the perimeter region 2955.

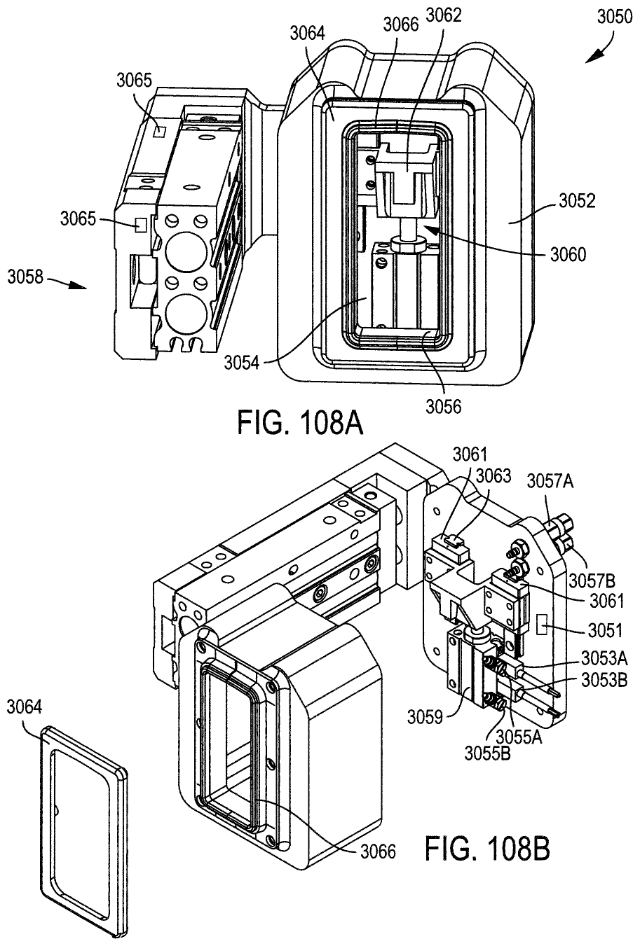

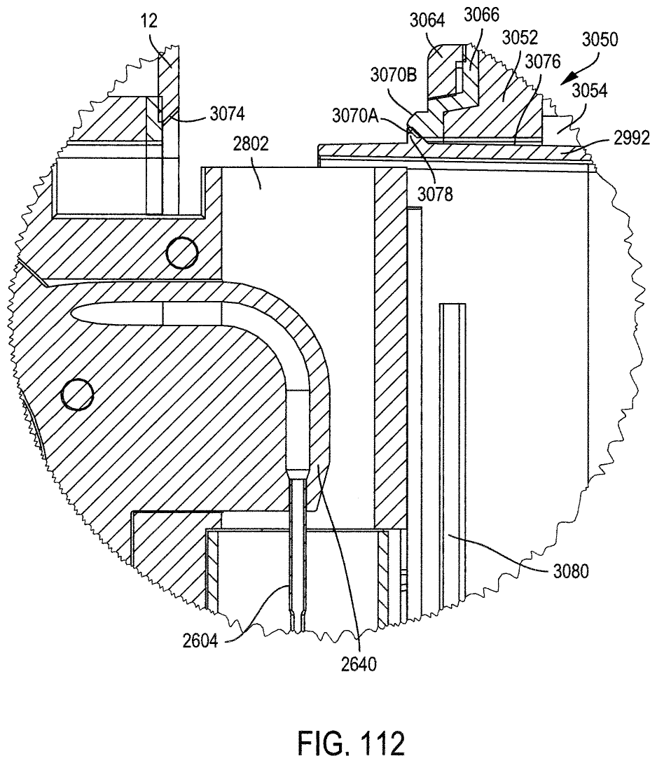

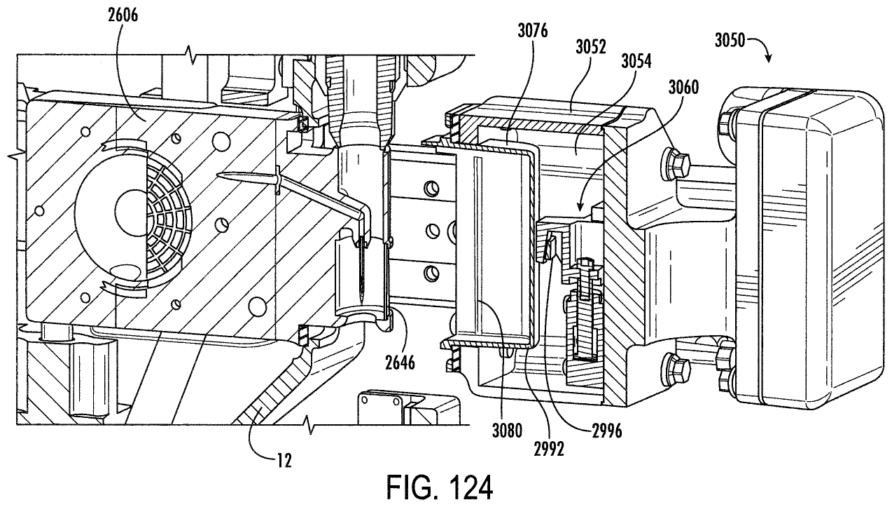

The cover 2992 may be retained in place by an interference fit, snap fit, magnets, etc. depending on the embodiment. In the example embodiment, the cartridge housing 2950 includes ridges 2951 (or alternatively recessed grooves). The interior surface of the cover 2992 may include latches 3080 (see, e.g., FIG. 112 and FIG. 124) which may snap in place or engage the ridges 2951 (or grooves) to couple the cover 2992 to the rest of the cartridge 2606. Preferably, the coupling formed by the interaction of the ridges 2951 and latches 3080 may be robust to inadvertent removal or dislodgement. When coupled in place, the cover 2992 may compress against the sealing member 2994. This forms an environmental seal which partitions and isolates the air flow channel 2802 and dispensing sharp 2604 from the surrounding environment outside of the cover 2992. The cartridge 2606 may be provided in a sterilized state and the cover 2992 may ensure that the air flow channel 2802 and the dispensing sharp 2604 maintain this state until use. The cover 2992 may remain in place when the cartridge 2606 is installed within a system 10 and may be removed by the system 10. In the example, the cover 2992 includes a catch 2996 for interfacing with a cover removal assembly 3050 (see, e.g., FIG. 108A-B) of the system 10.

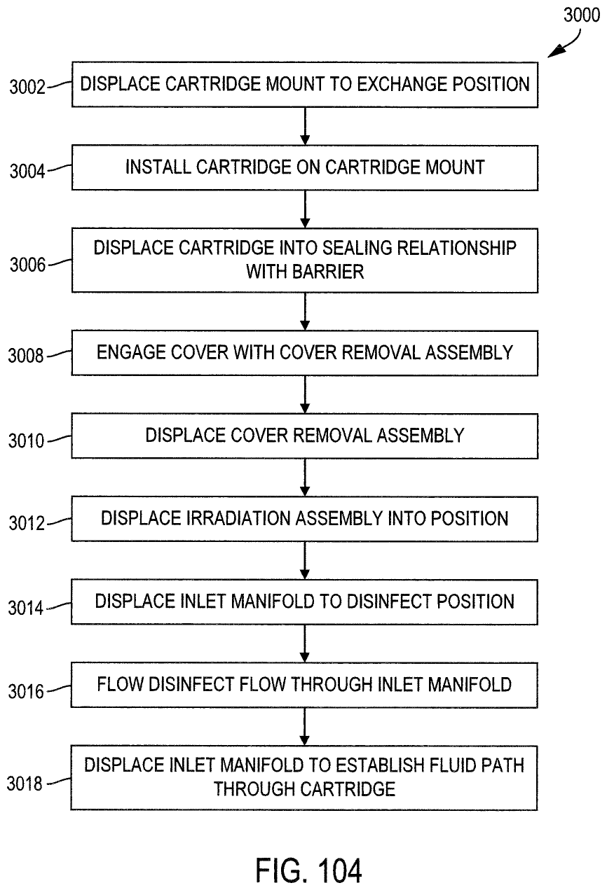

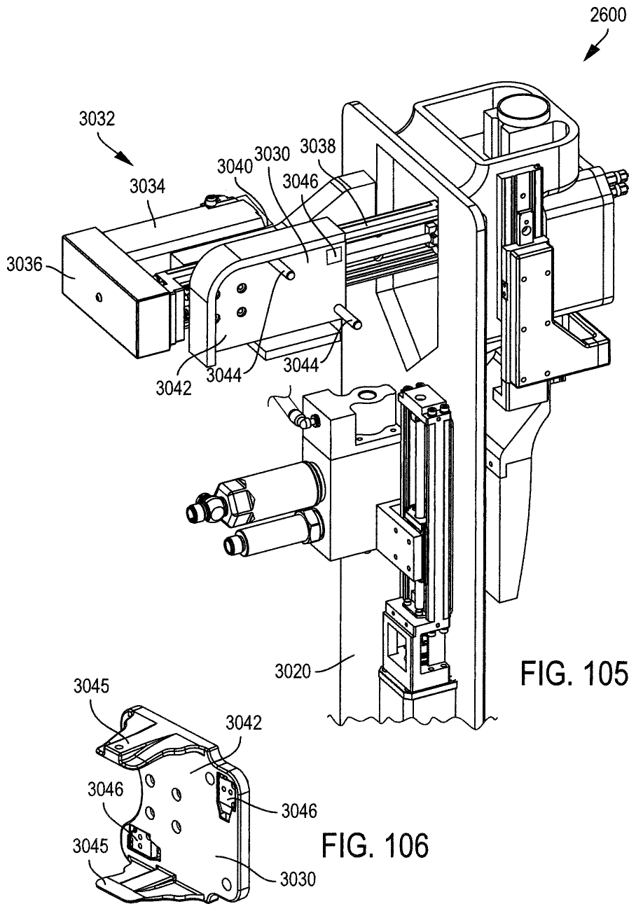

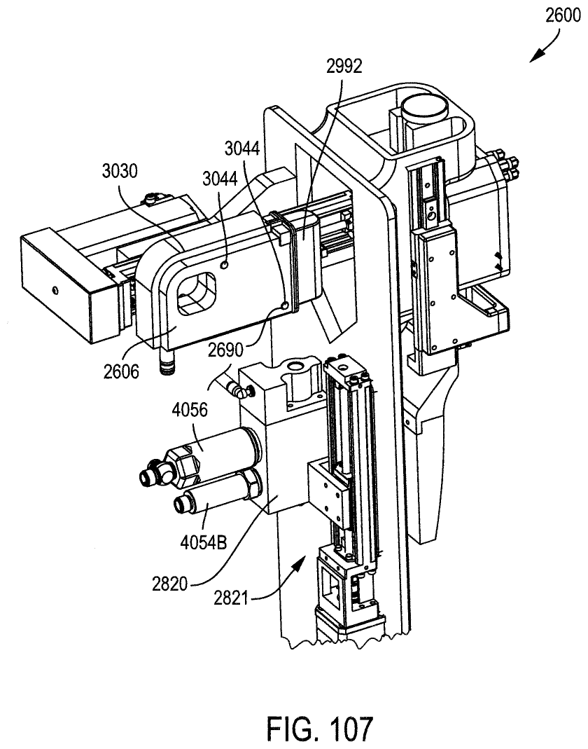

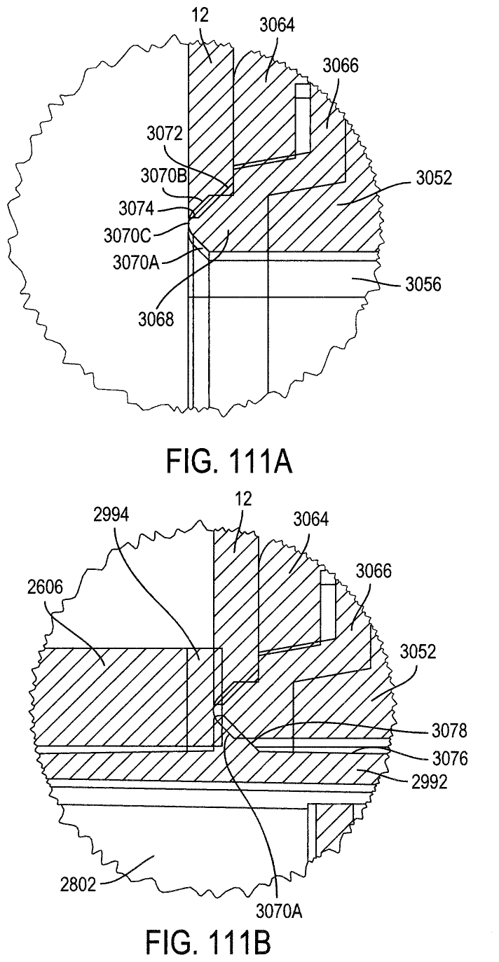

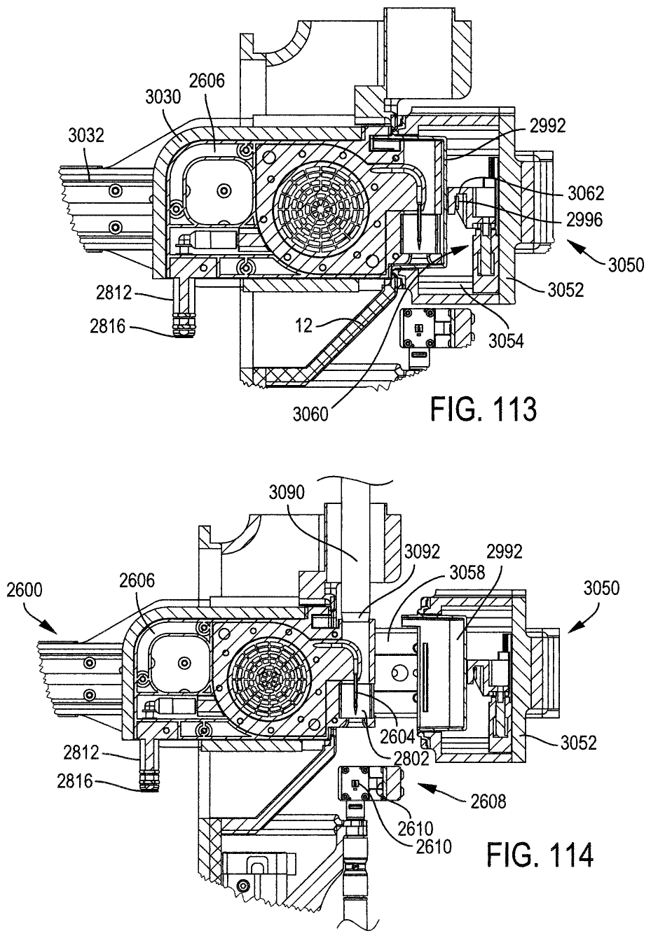

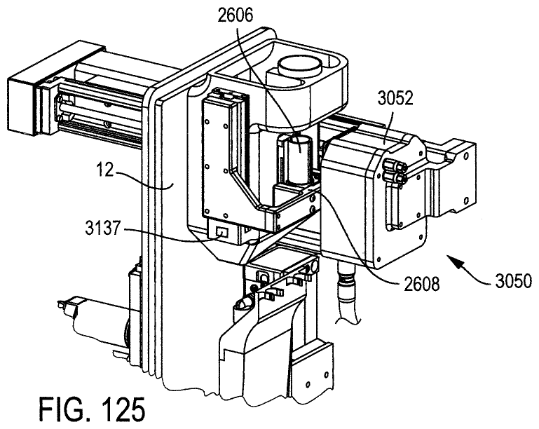

Referring now to FIG. 104, a flowchart 3000 depicting a number of example actions which may be executed to install a cartridge 2606 within a fill station 2600 is depicted. In block 3002, a cartridge mount or dock 3030 may be displaced to a cartridge exchange position (see, e.g., FIG. 105). A new cartridge 2606 may be installed into the cartridge mount 3030 in block 3004 (see, e.g., FIG. 107). In block 3006, the cartridge 2606 may be displaced to an access position (see, e.g., FIG. 110). This may be a position where the cartridge 2606 has been displaced into sealing relationship with a barrier (e.g. a portion of an enclosure 12 or wall 4301 of a processing compartment 4300). In some embodiments, the control system 15 may check the signal from one or more sensor monitoring the cartridge mount 3030 to confirm the cartridge 2606 is properly installed before displacing the cartridge 2606 to the access position. Where the cartridge 2606 includes a cartridge indicium such as a barcode, data-matrix, GS-1 code, etc., the indicium may be read and checked against an inventory database to verify the cartridge 2606 is acceptable for use. If the cartridge 2606 is unacceptable, the control system 15 may prohibit use of the cartridge 2606 and generate a notification to the user (e.g. via a user interface 6100). If the cartridge 2606 is verified acceptable, a cover removal assembly 3050 may, in block 3008 engage the cover 2992 on the cartridge 2606 (see, e.g., FIG. 113). Any trackers for odometry data (further described in relation to FIG. 106) relating to the cartridge 2606 may also be reset in block 3008. With the cover 2992 engaged, the cover removal assembly 3050 may be displaced away from the cartridge 2606 to separate the cover 2992 from the cartridge 2606 in block 3010 (see, e.g., FIG. 114). As further described in relation to FIGS. 111A-112, this may aseptically place the air flow channel 2802 and dispensing sharp 2604 into communication with a stringently controlled environment on the interior of the enclosure 12. In block 3012, the irradiation assembly 2608 may be displaced into alignment with the barrier 2646 on the cartridge 2606 (see, e.g., FIG. 125). The supply manifold 2820 may be displaced to a disinfect position in block 3014 (see, e.g., FIG. 126A). Disinfect fluid may flow through the supply manifold 2820 in block 3016. The supply manifold 2820 may be displaced so as to interrupt the frangible 2816 of the cartridge 2606 and establish fluid communication through the cartridge 2606 in block 3018 (see, e.g., FIG. 126B).

Referring now to FIG. 105 (see also, e.g., FIGS. 107, 125, 131, 133, 138, 139), an example fill station 2600 is depicted. The example fill station 2600 is depicted in isolation, however, typically would be included as one of a number of stations in a system 10 (see, e.g., FIG. 2). The example fill station 2600 includes portions which are partitioned into differing levels of environmental control. An enclosure 12 may form the partition between these portions. For purposes of illustration, the enclosure 12 is generally shown in abbreviated form in the forthcoming description. An example of an enclosure 12 which may be included in a system 10 is shown in unabbreviated from in FIG. 1 and various compartments forming the enclosure 12 are shown in FIG. 2.

Referring now primarily to FIG. 105, an example fill station 2600 which may be included in a system 10 is depicted. As shown, the fill station 2600 may include a cartridge dock or mount 3030. The cartridge mount 3030 may be attached to a cartridge displacement assembly 3032 which may include a motor 3034, transmission 3036, and an output actuator 3038 to which the cartridge mount 3030 is coupled. The cartridge displacement assembly 3032 may include at least one position sensor 3040. In certain examples, the cartridge displacement assembly 3032 may include a motor encoder. A linear potentiometer could be included and may monitor the position of the cartridge mount 3030. Any other suitable sensor may be used. A control system 15 may be in data communication with the at least one position sensor 3040 and motor 3034. The control system 15 may govern displacement of the cartridge mount 3030 based on the signals received from the at least one position sensor 3040.

The cartridge mount 3030 may include a receptacle 3042 sized to accept a cartridge 2606 (e.g. any of those described herein). The receptacle 3042 may be shaped such that the cartridge 2606 may only be installed in a prescribed orientation (e.g. be asymmetric as shown). The receptacle 3042 may include at least one guide 3044 which may help direct the cartridge 2606 into place during installation. Each of the at least one guide 3044 may be a positive projection or recess in the mount 3030 which interfaces with a cooperating feature of the cartridge 2606. Cartridges 2606 may include recesses or apertures which accept positive projections in the cartridge mount 3030 and/or may include positive projections which are received in recesses of the cartridge mount 3030. In the example embodiment in FIG. 105, the at least one guide 3044 in the cartridge mount 3030 includes a set of pins.

Referring now also to the example cartridge mount 3030 of FIG. 106, in some examples, cantilevered spring clips 3045 may be included to assist in retaining a cartridge 2606 on a cartridge mount 3030. Spring clips 3045 may inhibit misalignment and may center the cartridge 2606 within the receptacle 3042. When the cartridge 2606 is in place, the spring clips 3045 may be in a deflected state and may exert a bias against the cartridge 2606 which firmly holds the cartridge 2606 in place.

The receptacle 3042 of the cartridge mount 3030 may also include at least one cartridge presence sensor 3046. Though any suitable sensor may be utilized, the cartridge presence sensor 3046 may be a microswitch which is actuated when a cartridge 2606 is properly installed in the cartridge mount 3030. Alternatively, the cartridge 2606 may include a magnet or ferromagnetic body and the cartridge presence detector 3046 may include a magnetic sensor which may alter an output when the cartridge 2606 is properly installed in the cassette mount 3030 and the magnet or ferromagnetic body of the cartridge 2606 is in close proximity. A Hall effect sensor may, for example, be used in certain embodiments. Other sensors such as reflectivity based sensors may be used. Multiple presence sensors 3046 may be included in certain examples in order to help ensure a cartridge 2606 is not only present, but seated properly.

In some embodiments, at least one cartridge presence sensor 3046 may read a cartridge indicia which may contain information associated with the cartridge 2606 (e.g. a unique identifier for the cartridge 2606). In such examples, a bar code reader, data matrix reader (e.g. imager), RFID interrogator, NFC interrogator, etc. may be used. The cartridge 2606 may include a barcode, data matrix, RFID, or NFC tag as appropriate. Thus, the cartridge presence sensor 3046 may collect data related to the cartridge 2606 as an indication that a cartridge 2606 is installed in the cartridge mount 3030. The control system 15 may verify that use of the installed cartridge 2606 is permissible before, for example, commanding displacement of the output actuator 3038 or engaging the cover 2992 with a cover engagement assembly 3060 (see, e.g., block 3008 of FIG. 104). If the cartridge 2606 unique identifier is indicated in a database as having been used, recalled, or expired, use of the cartridge 2606 may be prohibited. The database may be updated to indicate that the cartridge 2606 has been used if the consumption of the cartridge 2606 is permissible. Information from the cartridge indicium may also be associated with the batch record for each bag 26 produced using that particular cartridge 2606. For example, the cartridge expiration date, serial no., lot no., etc. may be associated with the batch record for each bag 26 filled using the cartridge 2606. In some embodiments, odometry data relating to the cartridge 2606 may be tracked and included in the batch record for each bag 26. A tally of bags 26 filled or total amount of fluid dispensed using the cartridge 2606 may be tracked by the control system 15 and associated with batch records for each bag 26 filled through a particular cartridge 2606. Other information such as the period of time since last cartridge replacement, period of time since last disinfection, number of disinfections performed (and/or started) on a given cartridge 2606 may also be tracked and likewise stored in batch records for respective bags 26. Any cartridge 2606 odometry tracking may be reset upon installation of a new cartridge 2606 and verification that the cartridge 2606 is acceptable for use.

Referring now to FIG. 107, the filling station 2600 of FIG. 105 is depicted with a cartridge 2606 in place. The cartridge 2606 shown is exemplary and is constructed similarly to that depicted in FIGS. 99A-100B. The example cartridge 2606 also includes a cover 2992 similar to that described further in relation to FIGS. 101A-103C. As shown, the cartridge 2606 includes guides 2690 which may interact with the guides 3044 of the cartridge mount 3030. The example cartridge mount 3030 may include a clasp, clip, or other retainer which may be engaged with a cartridge 2606 to maintain the cartridge 2606 in the cartridge mount 3030 after a cartridge 2606 is installed. For example, the cartridge mount 3030 may be modified to include the spring clips 3045 of FIG. 106.

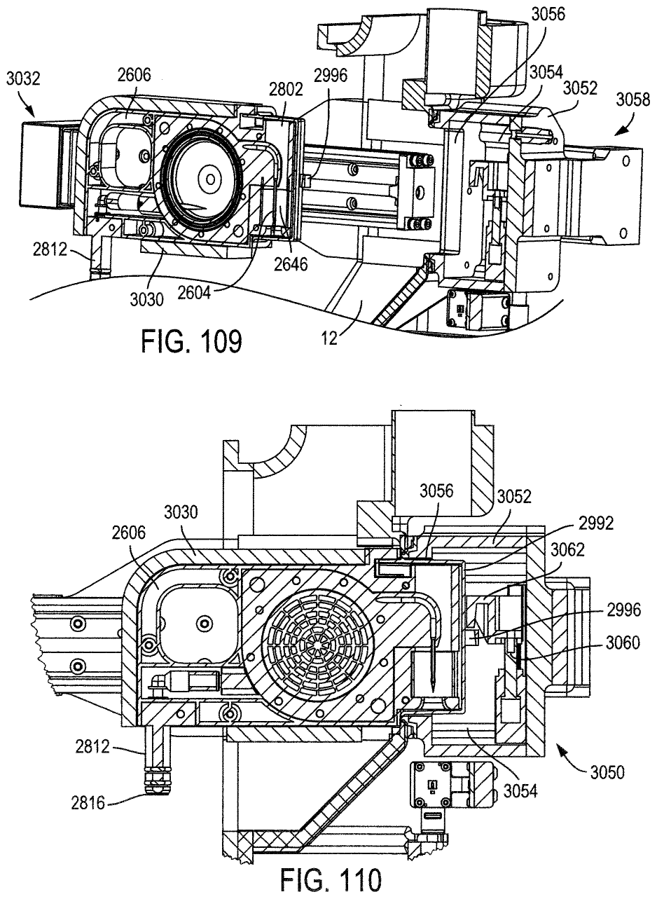

Referring now primarily to FIGS. 108-109, views of an example cover removal assembly 3050 and a perspective cross-sectional view of a portion of a fill station 2600 are respectively depicted. The cartridge 2606 in FIG. 109 is in a position where it has just been loaded onto a cartridge mount 3030. As mentioned in relation to FIG. 104, the cover 2992 of the cartridge 2606 may be removed by the cover removal assembly 3050. This may provide access to the air flow channel 2802, dispensing sharp 2604, and barrier 2646 of the cartridge 2606. As shown, an example cover removal assembly 3050 may include a housing 3052 with an interior bay 3054. The housing 3052 may define a bay ingress 3056. As shown in FIG. 110, when the cartridge 2606 is displaced to an access position by the cartridge displacement assembly 3032, the cover 2992 of the cartridge 2606 may be displaced at least partially into the bay 3054 via the bay ingress 3056.

Referring to FIGS. 108A-110, the housing 3052 may also be attached to a cover displacement assembly 3058. The cover displacement assembly 3058 may be actuated (e.g. pneumatically) by the control system 15 to displace the housing 3052 with respect to the enclosure 12. Where pneumatically actuated, the cover displacement assembly 3058 may be driven by a relatively high pressure pneumatic source (e.g. 80 psi). Pressure from the pneumatic source may be applied to the cover displacement assembly 3058 when the housing 3052 is displaced against the enclosure 12. This may ensure the sealing member 3066 of the housing 3052 is maintained in compression against the enclosure 12.

A cover engagement assembly 3060 (best shown in FIG. 108B) may be disposed within the bay 3054. In the example embodiment, the cover engagement assembly 3060 is positioned opposite the bay ingress 3056. A cover engagement assembly 3060 may be actuated (e.g. pneumatically) from a retracted state to a deployed state to retain a cover 2992 of a cartridge 2606. Once a cover 2992 is retained, the cover displacement assembly 3058 may be actuated (e.g. pneumatically) to disassociate the cover 2992 from a cartridge 2606 and displace the cover 2992 to a storage position.

The cover displacement assembly 3058 may include at least one position sensor 3065 which may output a signal indicative of the location of the housing 3052. In certain embodiments, two position sensors 3065 may be included and may output signals indicative that the housing 3052 has been displaced to respective ends of its displacement range. The control system 15 may be in data communication with any of the at least one position sensor 3065 and may command displacement of the housing 3052 via the cover displacement assembly 3058 based on data signals from the at least one position sensor 3065.

The example cover engagement assembly 3060 may include an engagement catch 3062 which may be displaced to engage and disengage a cover 2992 of a cartridge 2606. Catches 3062 may, for example, include a hook. In such embodiments, the cover 2992 may include a cover catch 2996 through or into which the hook may extend when the cover 2992 and cover engagement assembly 3060 are in an engaged state. The engagement catch 3062 of the cover engagement assembly 3060 may include wing assemblies 3061 which travel along bearing surfaces 3063 in the housing 3052. The engagement catch 3062 may be coupled to an actuator 3059 which may be operated to displace the engagement catch 3062. In the example, the actuator 3059 is a pneumatic actuator and may be selectively placed into communication with at least one pressure source (e.g. via one or more valve) to drive displacement of the engagement catch 3062. The housing 3052 includes positive and negative pressure inputs 3057A, B for connection to pneumatic pressure sources. Though not shown, pneumatic lines may run from the pressure inputs 3057A, B to pressure supply connections 3055A, B associated with the actuator 3059.

One or more position sensor 3053A, B may be included to monitor the position of the engagement catch 3062. In the example embodiment, two position sensors 3053A, B are included and may each output a signal indicative that the engagement catch 3062 is at a respective end of its displacement range (e.g. microswitches). In other embodiments, a linear potentiometer or the like may be included and may output a signal which varies in relation to the location of the engagement catch 3062 along its displacement range. Any other suitable sensor(s) may be included in alternative examples. The control system 15 may be in data communication with any of the at least one position sensor 3053A, B and may command displacement of the engagement catch 3062 based on data signals from the at least one position sensor 3053A, B.

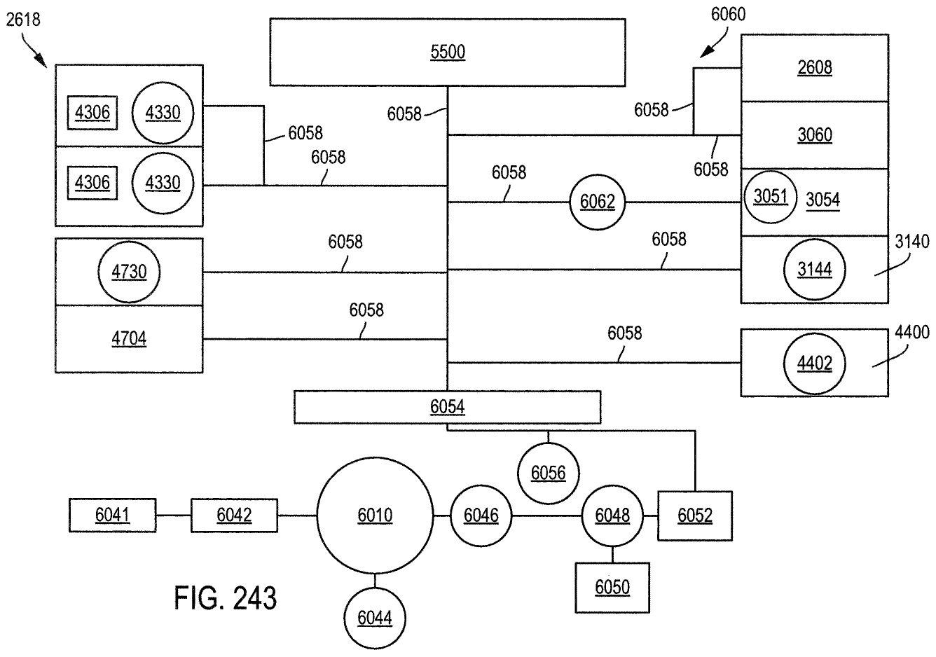

In certain examples, the bay 3054 may be placed into communication with the negative pressure supply or vacuum source 6010 (see, e.g., FIG. 243). This may be accomplished via toggling of a valve included in the cap removal assembly 3050 or in a negative pressure distribution assembly 6060 (see e.g. FIG. 243). In such embodiments, at least one pressure sensor 3051 may be included to monitor pressure within the bay 3054. The control system 15 may be in data communication with the at least one pressure sensor 3051. Pressure sensors 3051 are further described in relation to FIG. 111B.

Referring now to FIGS. 111A-112, a cartridge 2606 may be loaded to the cartridge mount 3030 exterior to an enclosure 12 of the system 10. Bags 26, however, may be filled on an interior of the enclosure 12 which is under more stringent environmental control. The enclosure 12 may include a fenestration 3074 through which a portion of the cartridge 2606 may extend so as to be accessible from the interior of the enclosure 12. In various embodiments, when a cartridge 2606 is installed in a fill station 2600, the cover 2992 may be removed in an aseptic manner to access the air flow channel 2802 and dispensing sharp 2604. Thus, the environment on the interior of the enclosure 12 may not be fouled when the air flow channel 2802 and dispensing sharp 2804 are accessed. Additionally, when cartridges 2606 are exchanged, environmental communication between the exterior side of the enclosure 12 and a more stringently controlled interior side of the enclosure 12 may be inhibited.

Referring to FIG. 111A, a cross-sectional view of a portion of an enclosure 12 and cap removal assembly 3050 is depicted. As shown, the cap removal assembly 3050 may include a sealing member 3066 which surrounds the bay ingress 3056 defined in the housing 3052 (see also FIG. 108B). The sealing member 3066 may be captured in place on the housing 3052 by a retainer plate 3064. The retainer plate 3064 may couple to the housing 3052 via fasteners, snap fit, or any other suitable manner with the sealing member 3066 disposed intermediate the housing 3052 and the retainer plate 3064. In other examples, the sealing member 3066 may be coupled to the housing 3052 in other manners. For example, the sealing member 3066 may be overmolded onto the housing 3052. In such examples, the retainer plate 3064 may be omitted.

The sealing member 3066 may include a ridge 3068 which may surround the bay ingress 3056. The ridge 3068 may include an interior sidewall 3070A and an exterior sidewall 3070B which may be angled toward one another. The ridge 3068 may also include an intermediate span 3070C extending from the interior sidewall 3070A to the exterior sidewall 3070B. The intermediate span 3070C may be the portion of the ridge 3068 most distal to the housing 3052 and may be a rounded span (as shown) or plateau. In other examples, the interior and exterior sidewalls 3070A, B may extend until they meet substantially at a point.

When a cartridge 2606 is exchanged, the housing 3052 of the cover removal assembly 3050 may be positioned such that the sealing member 3066 is compressed against the walls 3072 of the fenestration 3074 in the enclosure 12. This may prevent communication between the environment on the exterior of the enclosure 12 and the environment on the interior of the enclosure 12. That is, the seal may isolate the interior of the enclosure 12 from the exterior of the enclosure 12. As shown, the walls 3072 may be contoured to match the angle of the exterior sidewall 3070B of the sealing member 3066. When the cover removal assembly 3050 is sealed around the fenestration 3074, the seal may be primarily or entirely established by compression of the exterior sidewall 3070B against the walls 3072 of the fenestration 3074.

Referring now to FIG. 111B, as a cartridge 2606 is displaced to the access position by the cartridge displacement assembly 3032, the cover 2992 may project through the fenestration 3074 and into the bay 3054 of the housing 3052 of the cover removal assembly 3050. When the cartridge 2606 is in the access position, the sealing member 3066 of the cover removal assembly 3050 may establish a seal around the exterior surface 3076 of the cover 2992. As shown, the cover 2992 may include a protrusion 3078 on its exterior surface 3076 with a face contoured to match the angle of the interior sidewall 3070A of the sealing member 3066. The seal may be primarily or entirely established by compression of the interior sidewall 3070A against the protrusion 3078 on the exterior surface 3076 of the cover 2992. Once this seal is formed, the bay 3054 and portion of the exterior surface 3076 of the cover 2992 previously exposed to the exterior of the enclosure 12 may be isolated from the interior as well as the exterior of the enclosure 12.

In certain examples, the control system 15 may place the bay 3054 into communication with a negative pressure source (e.g. via toggling of at least one valve). The output of at least one pressure sensor 3051 (see, e.g., FIG. 108B) in communication with the bay 3054 may be monitored by the control system 15. The signal from the one or more pressure sensor 3051 may be monitored to ensure that a proper seal has been formed between the cover removal assembly 3050 and a cover 2992. In the event a robust seal is formed between the sealing member 3066 and the cover 2992, the negative pressure in the bay 3054 will exhibit minimal decay over time. In the event that the data from the pressure sensor 3051 indicates pressure is steady (decay rate is less than a threshold), the control system 15 may proceed to remove the cover 2992 from the cartridge 2606. Negatively pressurizing the bay 3054 may also assist in retaining a cover 2992 against the cover removal assembly 3050.

In the event the pressure sensor(s) 3051 indicates the pressure in the bay 3054 is above a threshold, the control system 15 may prohibit actuation of the cover engagement assembly 3060. Additionally or alternatively, in the event that data from the pressure sensor 3051 indicates the pressure within the bay 3054 is decaying beyond a threshold rate, the control system 15 may prohibit removal of the cover 2992. In some examples, a notification may be generated for display on a user interface if one or more predefined pressure characteristic is not observed with data from the one or more pressure sensor 3051. In some examples, the control system 15 may attempt to re-establish a seal against the cover 2992 up to a preset number of retry attempts. The cartridge 2606 may, for example, be backed away from and advanced back toward the cover removal assembly 3050 by commanding actuation of the cartridge displacement assembly 3032. The control system 15 may also generate a notification and potentially prohibit one or more functionality of the system 10 if the pressure in the bay 3054 rises above a threshold after the cover 2992 is engaged and removed by the cover removal assembly 3050.

As mentioned in relation to FIG. 103A, example cartridges 2606 may include a cartridge sealing member 2994. The cartridge sealing member 2994 may establish a seal against the exterior surface 3076 of the cover 2992 when the cover 2992 is in place on the cartridge 2606. In the example, the cartridge sealing member 2994 is compressed against the protrusion 3078 on the exterior surface 3076 of the cover 2992.

Referring primarily to FIG. 111B, the cartridge 2606 may establish a seal, via the cartridge sealing member 2994, around the fenestration 3074 in the enclosure 12 when the cartridge 2606 is in the access position. This may isolate the environment on the exterior of the enclosure 12 and areas of the cartridge 2606 exposed to that environment from the interior of the enclosure 12.

Referring now to FIG. 112, when the cover 2992 is removed from the cartridge 2606, the sealing member 3066 of the cover removal assembly 3050 may maintain a sealing relationship against the cover 2992. Thus, the surfaces of the cover 2992 and bay 3054 which were exposed to the environment on the exterior of the enclosure 12 may remain isolated from the interior of the enclosure 12. The sealing member 3066 may be displaced away from the walls 3072 of the fenestration 3074 when the cover 2992 is removed from the cartridge 2606. The seal established around the fenestration 3074 by the cartridge sealing member 2994 may, however, be maintained. This may prevent communication between the interior and exterior of the enclosure 12. The exterior sidewall 3070B of the sealing member 3066 of the cover removal assembly 3050 may only be exposed to conditions on the interior of the enclosure 12.

The cartridge 2606 may be provided terminally sterilized (e.g. via gamma, ethylene oxide, etc.). When the cover 2992 is in place on the cartridge 2606, the seal between the cartridge sealing member 2994 and cover 2992 may ensure the interior of the cover 2992 and unexposed portions of the cover 2992 remain sterile. The portion of the cartridge sealing member 2994 against which the cover 2992 seals may also be maintained sterile. As these regions are in a sterile state before removal of the cover 2992, the environment on the interior of the enclosure 12 may not be negatively impacted when the cover 2992 is doffed from the cartridge 2606. Thus, the air flow path 2802 and dispensing sharp 2604 of the cartridge 2606 may be introduced into the interior of the enclosure 12 in aseptic manner.

A cartridge 2606 may also be aseptically removed from the filling station 2600 with the example arrangements described herein. The cover displacement assembly 3058 of the cover removal assembly 3050 may displace the cover 2992 back into engagement with the cartridge 2606 (into the state depicted and described in relation to FIG. 111B). In this position, the interior sidewall 3070A of the sealing member 3066 of the cover removal assembly 3050 may continue to isolate the bay 3054 and received portion of the cover 2992 from the remainder of the system 10. The exterior sidewall 3070B may establish a seal isolating the interior of the enclosure 12 from the exterior of the enclosure 12. The cartridge 2606, with a reinstalled cover 2992, may then be displaced by the cartridge displacement assembly 3032 to an exchange position. The seal between the cartridge sealing member 2994 and the enclosure 12 may be eliminated as the cartridge 2606 is brought to the exchange position (see, e.g., FIG. 111A). Isolation between the exterior of the enclosure 12 and interior of the enclosure 12 may be maintained by the seal created between the exterior sidewall 3070B of the sealing member 3066 and the walls 3072 of the fenestration 3074.

Referring now to FIG. 113, a cross-sectional view of a portion of a fill station 2600 with a cover engagement assembly 3060 engaged with a cover 2992 is depicted. As shown, the cover engagement assembly 3060 may include an engagement catch 3062. The cover 2992 may include a cover catch 2996. The engagement catch 3062 is in the shape of a hook and the exemplary cover catch 2996 is a bridge of material extending proud of the exterior surface 3076 of the cover 2992. The cover catch 2996 may be a pocket or recess in alterative examples. The hook may be displaced under the bridge of material to place the cover engagement assembly 3060 into an engaged state with the cover 2992 in the example embodiment. As shown, the hook may include a ramped face 3082. Thus, the hook may taper thicker as distance from its unsupported end increases. As the hook is driven into the cover catch 2996, the cover 2992 may be cinched up against the sealing member 3066 of the cap removal assembly 3050. This may help to ensure the seal generated between the cover 2992 and the sealing member 3066 is robustly formed.

As mentioned above, in certain embodiments, the bay 3054 may be negatively pressurized and the pressure in the bay 3054 may be monitored by at least one pressure sensor 3051. Data from the pressure sensor 3051 may be analyzed by the control system 15 to verify an acceptable seal has been formed. The negative pressure within the bay 3054 may also assist in holding the cover 2992 firmly against the sealing member 3066. Data from the engagement catch position sensors 3053A, B (see, e.g., FIG. 108B) may be monitored to ensure the engagement catch 3062 is in an engaged state with the cover catch 2996. For example, in some embodiments, the engagement catch 3062 may have an over-travel position which would be reached in the event the cover catch 2996 does not present an interference. The control system 15 may prevent an attempt to remove the cover 2992 in the event data from the engagement catch position sensors 3053A, B indicates the engagement catch 3062 is in the over-travel position (or failed to displace from its retracted state, or is not within an expected position range). In some embodiments, the control system 15 may command one or more retry actuations of the cover engagement assembly 3060 (up to an allotted cap) before generating a notification.

Referring now to FIG. 114, a cross-sectional view is depicted of a portion of a fill station 2600 where the cover 2992 has been removed from a cartridge 2606 in an access position. As discussed above in relation to FIGS. 111A-112, the air flow channel 2802 and dispensing sharp 2604 may be exposed to the interior environment of the enclosure 12 in an aseptic manner when the cover 2992 is removed from the cartridge 2606. Once the air flow channel 2802 is accessible, a clean air output 3090 (best shown in FIG. 118) may be displaced against an upstream end of the air flow channel 2802. In certain embodiments, the clean air output 3090 may include a tapered end 3092 which seats against the upstream end of the air flow channel 2802. In some examples, a sealing member may be included on one or both of the tapered end 3092 and upstream end of the air flow channel 2802. Depending on, for example, the pressure and flow rate desired through the air flow channel 2802, sealing member(s) may be omitted.

Clean air may be provided to the processing chamber 4300 and clean air output 3090 from a laminar air flow source. Clean air may flow from the clean air output 3090 and through the air flow channel 2802 in a highly laminar character. The pressure, flow rate, particle count, etc. of the clean air passing through the air flow channel 2802 may be equivalent to requirements of a desired clean room standard (e.g. ISO 5 set points). Thus, the air flow channel 2802 may be held to a stringent level of environmental control. This level may, moreover, be more stringent than that imposed on the interior volume of the enclosure 12 with which the air flow channel 2802 communicates. Thus, a small fill zone volume which is under relatively strict environmental control may be established.

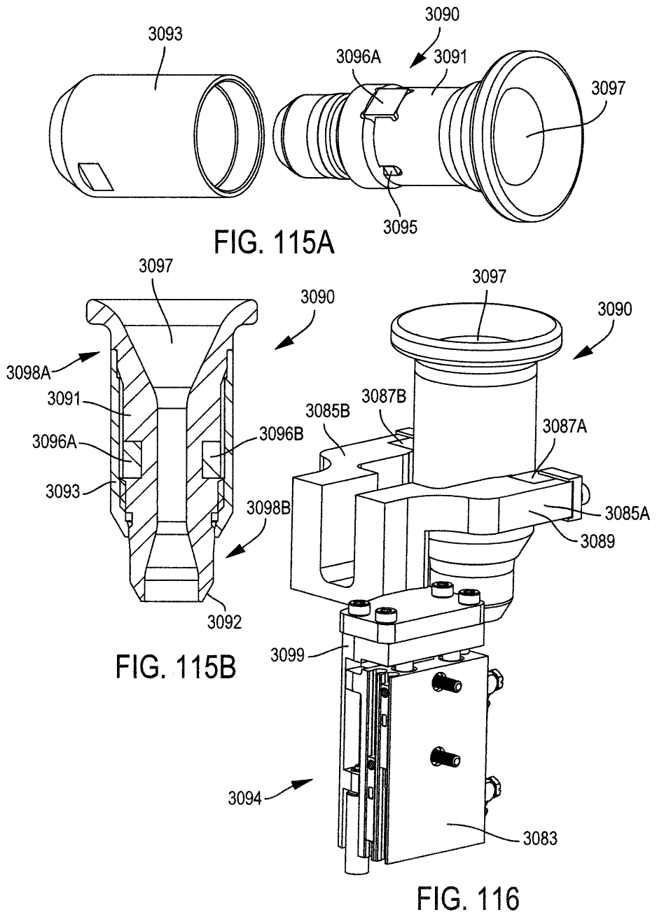

Referring now to FIGS. 115A-B, an exemplary clean air output 3090 is depicted. As shown, the clean air output 3090 may include a main body 3091 covered by a sleeve 3093. A set of magnets 3096A, B and a tracking body 3095 may be coupled to the main body 3091. The tracking body 3095 may be magnetic or ferromagnetic in certain examples. A bore 3097 may extend through the main body 3091. The bore 3097 may taper from a largest diameter at a first end region 3098A to a smallest diameter. The bore 3097 may taper from the smallest diameter to an intermediate diameter at a second end region 3098B opposite the first end region. The bore 3097 may be maintained at the smallest diameter through a portion of the main body 3091 intermediate the first and second end region 3098A, B. The diameter of the bore 3097 may be substantially constant over the region where the tapered end 3092 of the main body 3091 extends. Thus, the bore 3079 may be arranged as a Venturi which increases flow rate while decreasing pressure. This may assist in keeping flow substantially laminar as it passes through an airflow channel 2802 of a cartridge 2606 and into the processing chamber 4300.

In some embodiments, turbulence reducing flow conditioners (e.g. vanes) may be included at the upstream side of the bore 3097 (or may be separate the clean air output 3090 and positioned upstream of the bore 3097). Additionally, in some embodiments, an irradiation assembly 2608 may also or instead be included for the clean air output 3090. The irradiation assembly 2608 may include antimicrobial light emitters 2610 (e.g. UVC LEDs) positioned around the exterior of a portion of the clean air output 3090. In such examples, the clean air output 3090 may include an irradiation transmissive barrier 2646 (see, e.g., FIG. 101B) which is in the illumination field of the antimicrobial light emitters 2610 throughout the displacement range of the clean air output 3090.

Referring now also to FIGS. 116-118, the clean air output 3090 may be displaced via clean air output actuation assembly 3094 through a non-contact coupling. The clean air output actuation assembly 3094 may include a displacement stage 3099 with a yoke body 3089 coupled thereto. The displacement stage 3099 may be translationally displaced by a pneumatic actuator 3083 (though any other variety of actuator may be utilized). The yoke body 3089 may include a set of arms 3085A, B each including a respective magnet 3087A, B of the clean air output actuation assembly 3094. As the displacement stage 3099 is displaced, the clean air output 3090 may displace in tandem due to the magnetic attraction between the magnets 3096A, B of the clean air output 3090 and the magnets 3087A, B on the yoke body 3089.

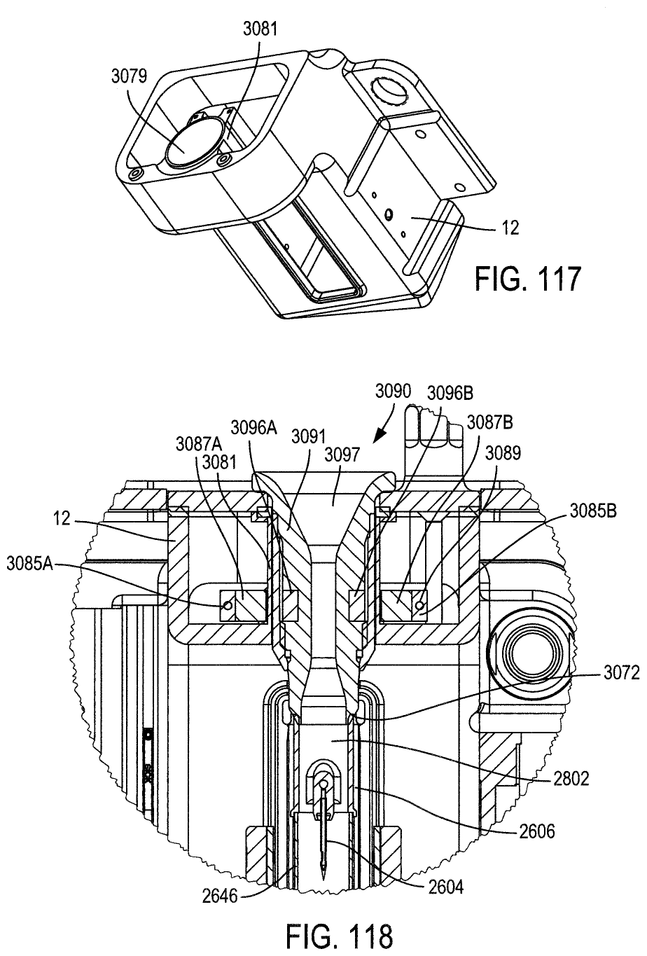

Referring now primarily to FIGS. 117-118, the enclosure 12 of the system 10 may be disposed intermediate the yoke body 3089 and the clean air output 3090. Thus, the clean air output actuation assembly 3094 may be disposed outside of the processing chamber 4300 and displace the clean air output 3090 inside the processing chamber 4300 through the enclosure 12 wall. A portion of the enclosure 12 is depicted in FIG. 117. A clean air output guide 3081 may be defined in the enclosure 12. The guide 3081 may include an interior passage 3079 with a diameter slightly larger than the outside diameter of the sleeve 3093 of the clean air output 3090. The clean air output 3090 may displace within this interior passage 3079. The yoke body 3089 may travel along the exterior surfaces of the guide 3081. The guide 3081 may be sufficiently thin that a robust magnetic coupling through the guide 3081 is created between the yoke body 3089 and the clean air output 3090.

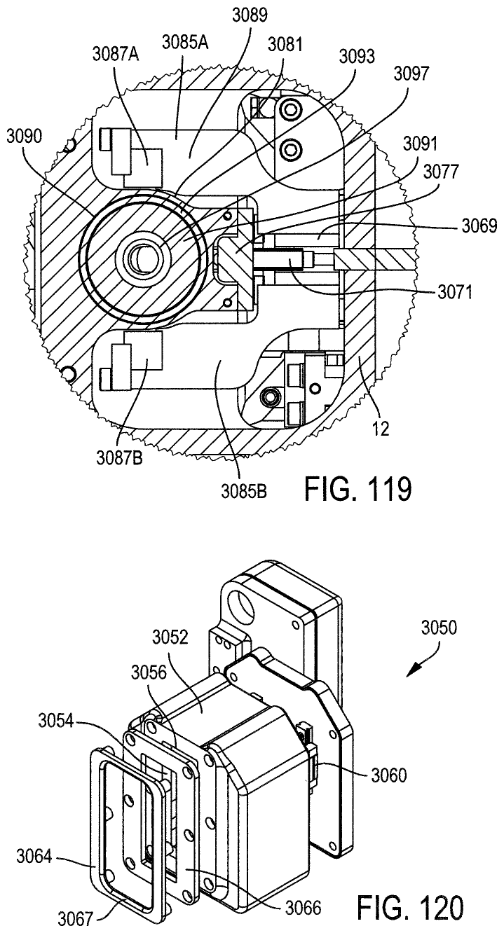

Referring now also to FIG. 119, the clean air output 3090 may be displaced between a raised position and a lowered position (see, e.g., FIG. 118) by displacing the yoke body 3089 via the pneumatic actuator 3038. In the raised position, the clean air output 3090 may provide clearance for a cover removal assembly 3050 to displace toward and away from a cartridge 2606. In the lowered position, the tapered end 3092 of the clean air output 3090 may be seated against the end of the air flow channel 2802 of the cartridge 2606. A sensor mount 3077 may be coupled to the guide 3081. One or more clean air output sensor 3071 may be coupled to the sensor mount 3077. The clean air output sensor(s) 3071 may be inductive sensors which generate an output signal which varies as the tracking body 3095 displaces. In some embodiments, a first clean air output sensor 3071 may be included substantially in line with the position of the tracking body 3095 when the clean air output 3090 is in the lowered position. A second clean air output sensor 3071 may be included and be substantially in line with the position of the tracking body 3095 when the clean air output 3090 is in the retracted or withdrawn position. The control system 15 may govern operation of the pneumatic actuator 3038 based on the output signals from the clean air output sensors 3071. The control system 15 may generate a fault if the clean air output sensors 3071 fail to register displacement of the clean air output 3090 when the actuator 3038 is powered. The yoke body 3089 may include a recessed channel 3069 which provides clearance for the clean air output sensors 3071 over the stroke of the yoke body 3089 between the raised and lowered positions.

Referring now to FIG. 120, a portion of an example alternative cover removal assembly 3050 is depicted. The cover displacement assembly 3058 is hidden in FIG. 120 for sake of illustration, but may be the same as that depicted in FIGS. 108A-B. As shown, the housing 3052 may include a sealing member 3066 which is substantially planar. The sealing member 3066 may be coupled to the housing 3052 via a retainer plate 3064 and fasteners (though could alternatively be overmolded to the housing 3052). The retainer plate 3064 may include a central aperture 3067 which leaves a portion of the sealing member 3066 adjacent the bay ingress 3056 of the housing 3052 exposed. The walls of the central aperture 3067 may be chamfered or rounded.

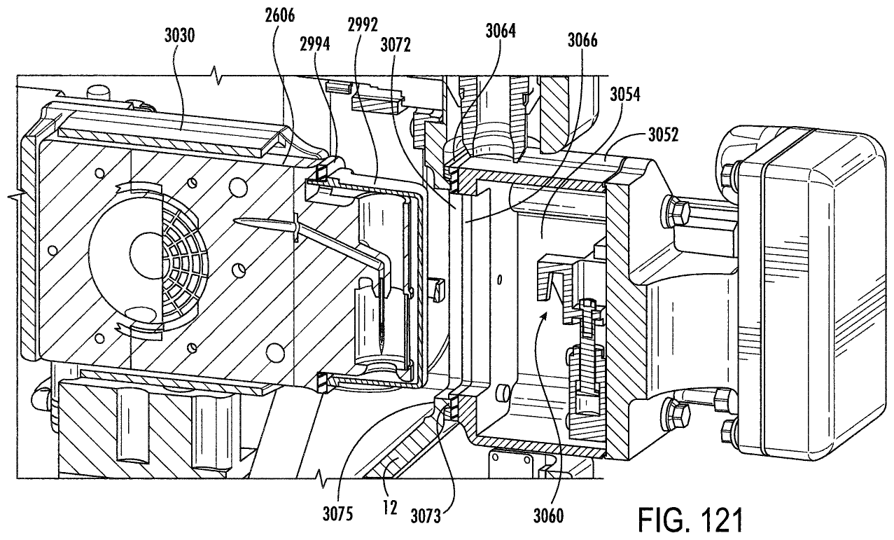

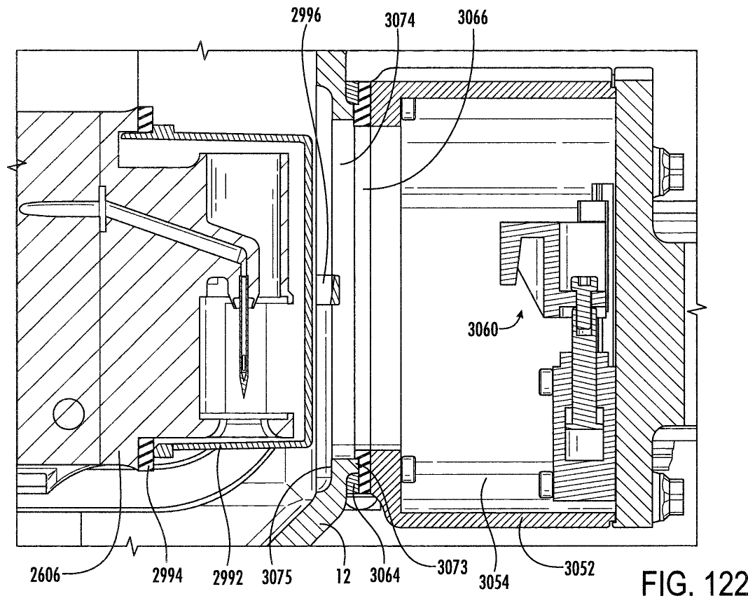

Referring now to FIGS. 121-122, cross-sectional views of a portion of an example fill station 2600 are depicted. As shown, a cartridge 2606 is in place on the cartridge mount 3030, but has yet to be advanced to the access position. The cover removal assembly 3050 is that depicted in FIG. 120. As shown, the cover removal assembly 3050 may be advanced (via commands from the control system 15 to a cover displacement assembly 3058) to form a seal with respect to the enclosure 12. The example enclosure 12 includes a fenestration 3072 with a raised wall 3073 surrounding the periphery of the fenestration 3072 on the interior side of the enclosure 3072. The sealing member 3066 of the cover removal assembly 3050 may compress against the raised wall 3073 to establish a seal which isolates the interior of the enclosure 12 from the exterior of the enclosure 12. As shown, the edge of the raised wall 3073 most distal the fenestration 3072 may be contoured to match the central aperture 3067 of the retaining plate 3064. When the sealing member 3066 is in a sealing relationship with the raised wall 3073, a portion of the sealing member 3066 immediately adjacent the bay ingress 3056 to the bay 3054 of the housing 3052 may remain exposed.

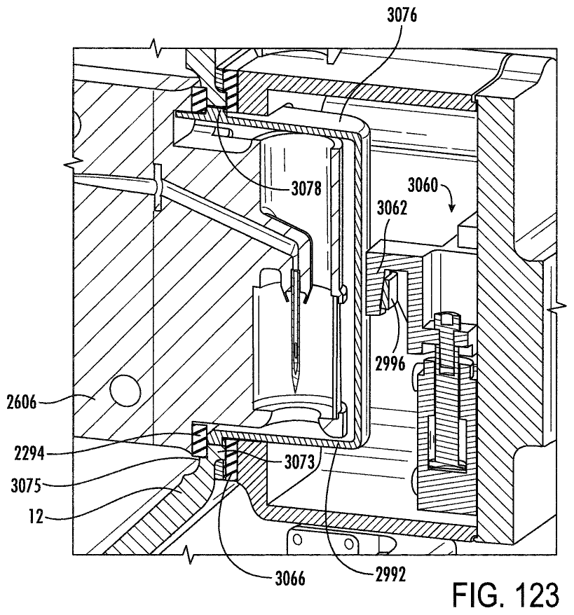

Referring now to FIG. 123, as the cartridge 2606 is advanced to the access position by the cartridge displacement assembly 3032, the sealing member 2994 of the cartridge 2606 may compress against a sealing surface 3075 of the enclosure 12. The sealing surface 3075 may surround the fenestration 3072. As the sealing member 2994 compresses against the sealing surface 3075 a seal which isolates the exterior of the enclosure 12 from the interior of the enclosure 12 may be formed. The exposed region of the sealing member 3066 on the housing 3052 of the cover removal assembly 3050 may compress against a face of a protrusion 3078 on the exterior surface 3076 of the cover 2992. The cover engagement assembly 3060 may be actuated via a command from the control system 15 to drive the engagement catch 3062 into an engaged state with the cover catch 2996. The control system 15 may then command displacement of the cover displacement assembly 3058 (see, e.g., FIG. 108A) to remove the cover 2992 from the cartridge 2606.

Referring now to FIG. 124, once the cover 2992 has been removed from the cartridge 2606, the seal between the sealing member 3066 on the housing 3052 of the cover removal assembly 3050 and the raised wall 3073 may be absent. The exterior of the enclosure 12 is still prevented from communicating with the interior of the enclosure 12 by the seal formed between the cartridge sealing member 2994 and the sealing surface 3075. Portions of the cover 2992 exposed to the environment on the exterior of the enclosure 12 are sealed within the bay 3054 of the housing 3052 of the cover removal assembly 3050 by the seal between the sealing member and the cover 2992 protrusion 3078.

Referring now primarily to FIG. 125, the irradiation assembly 2608 of the fill station 2600 may be displaceable between a retracted positions (shown in, e.g., FIG. 114) and a deployed position (shown in FIG. 125). The irradiation assembly 2608 may be coupled to an actuation assembly 3139 which may be commanded, via the control system 15, to displace the irradiation assembly 2608. Commands from the control system 15 may be based on a data signal from at least one position sensor 3137 monitoring the position of the irradiation assembly 2608. The irradiation assembly 2608 may be retracted so as to not infringe on the displacement path of the housing 3052 of the cover displacement assembly 3050 when a cartridge 2606 is being installed or exchanged. Once a cartridge 2606 is in the access position and the cover 2992 has been removed, the irradiation assembly 2608 may be displaced to the deployed position. In the deployed position, the light emitters 2610 of the irradiation assembly 2608 may be aligned with the barrier 2646 (see, e.g., FIG. 124) of the cartridge 2606. In some embodiments, the light emitters 2610 may be mounted on a rigid flex PCB including a span of slack which may support displacement of the irradiation assembly 2608 between the retracted and deployed positions.

Referring now to FIGS. 126A-B, once a cartridge 2606 has been displaced to the access position, the connection port 2812 of the cartridge 2606 may be placed in fluid communication with a fluid source 2810 (see, e.g., FIG. 90). As shown in FIG. 126A, a fill station 2600 may include a supply manifold 2820 including a receptacle 2818 for receiving the connection port 2812. The supply manifold 2820 may be disposed on the exterior of the enclosure 12. The supply manifold 2820 may be coupled to a manifold displacement assembly 2821 (see, e.g., FIG. 107). The receptacle 2818 may include a spike 2822 at an end thereof. A disinfect outlet channel 2824 may also be included in the sidewall of the receptacle 2818.

Referring primarily to FIG. 126A, the supply manifold 2820 may be displaced toward the connection port 2812 until the connection port 2812 is in a partially received state within a receptacle 2818 of the supply manifold 2820. Displacement of the supply manifold 2820 may be governed by commands to the manifold displacement assembly 2821 (see, e.g., FIG. 107) generated by the control system 15. When in the partially received state, the frangible 2816 of the connection port 2812 may be positioned such that the disinfect outlet channel 2824 is intermediate the frangible 2816 and the spike 2822. A first sealing member 2814A of the connection port 2812 may form a seal against the sidewall of the receptacle 2818. Disinfect fluid may be passed through the receptacle 2818 and out of the disinfect outlet channel 2824 for a predefined period of time. In certain examples, the disinfect fluid may be injection quality water which has been heated to at least 60° C. This may disinfect portions of the connection port 2812 and receptacle 2818 before fluid communication with the sterilized interior of the cartridge 2606 is established.

In various examples, one or more fluid characteristic sensors 4054B, 4056 (see, e.g., FIG. 107) may be included in the supply manifold 2820. At least one temperature sensor may for example be included. Data from any temperatures sensors may be monitored by the control system 15 to ensure the disinfection fluid is maintained in a desired temperature range. In the event data from any temperature sensors indicates that the disinfect fluid temperature does not conform to the desired range, the control system 15 may generate an alert and/or prolong the time period which disinfect fluid is passed through the receptacle 2818. The fluid characteristic sensors 4054B, 4056 may include at least one pressure sensor in certain examples. A pressure sensor may provide information indicative of the state of a filter 2609 (see, e.g., FIG. 101B) of the cartridge 2606. If a pressure sensor outputs a signal outside of an expected range, the control system 15 may determine the filter 2609 requires replacement (e.g. has fouled or become compromised). The control system 15 may generate an alert and require replacement of the cartridge 2606 before further bags 26 may be filled by the system 10. The alert may differ in the event that the pressure is in excess of the expected range or falls below that range. The fluid characteristic sensors 4054B, 4056 may include at least one conductivity sensor in certain examples. Data from any conductivity sensors may be monitored by the control system 15 to ensure the fluid entering the receptacle or cartridge 2606 conforms to preset conductivity criteria. In the event data from any conductivity sensors indicates that the fluid does not conform to the conductivity criteria, the control system 15 may generate an alert. Additionally, the control system 15 may ensure that any bag 26 which received the non-conforming fluid is handled so as to inhibit its use (e.g. labeled as rejected and/or segregated from other bags 26).

Referring now also to FIG. 126B, after flow of disinfection fluid through the receptacle 2818 and over the connection port 2812, the supply manifold 2820 may be displaced toward the cartridge 2606 until the connection port 2812 is in a fully installed position within the receptacle 2818. As this occurs, the spike 2822 may puncture the frangible 2816 sealing the connection port 2812. This may place the fluid source 2810 (see, e.g., FIG. 90) in fluid communication with the dispensing sharp 2604 through the cartridge 2606. The sealing members 2814A, B of the connection port 2812 may advance along the receptacle 2818 as the connection port 2812 is brought to the fully installed position. In this position, the disinfect outlet channel 2824 may be upstream of the sealing member 2814A, B and flow from the spike 2822 to the disinfect outlet channel 2824 may be inhibited.

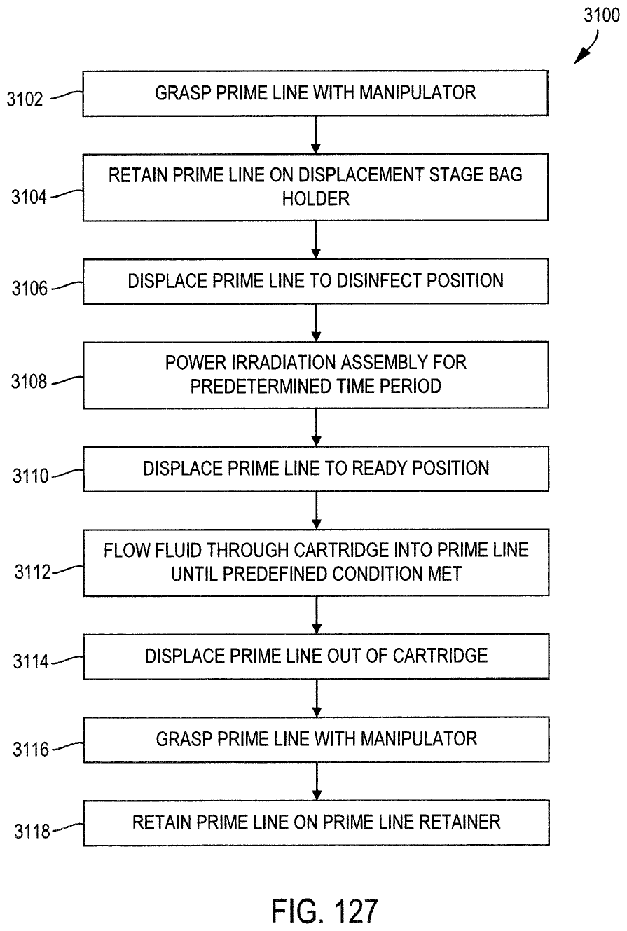

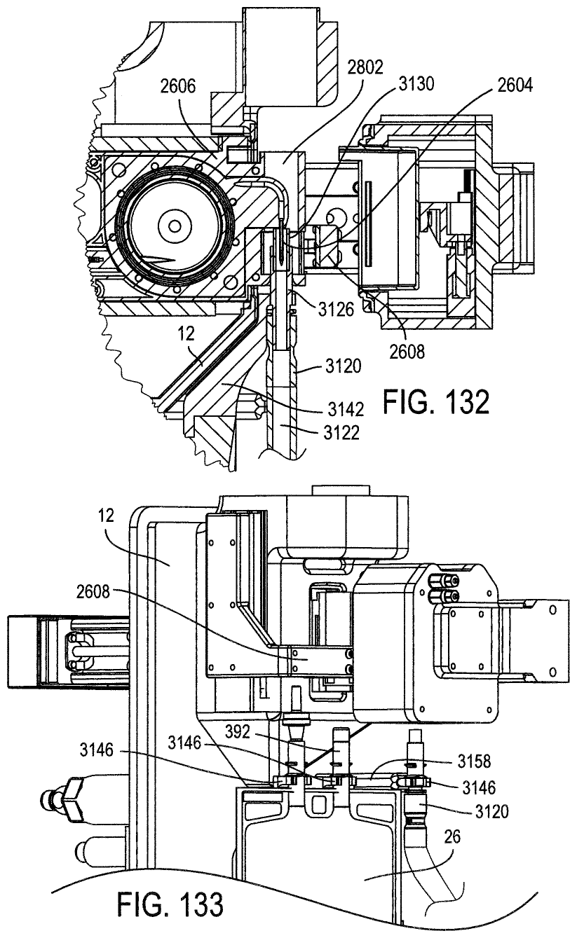

Referring now to FIG. 127, a flowchart 3100 detailing a number of example actions which may be executed to prime a freshly installed cartridge 2606 at a fill station 2600 is depicted. As shown, in block 3102 a priming line 3120 within the enclosure 12 may be grasped by a robotic gripper or manipulator (see, e.g., bag retainer assemblies 4306 of FIG. 68A). The priming line 3120 may be displaced to and retained on a displacement stage 3142 in block 3104 (see, e.g., FIG. 131). The robotic manipulator may also be displaced, in block 3104, to a retracted position after depositing the priming line 3120 on the fill displacement stage 3142. The priming line 3120 may be displaced, in block 3106, to a disinfect position. In block 3108, an irradiation assembly 2608 may be powered for a predetermined period of time. The prime line 3120 may be displaced to a priming position in block 3110 (see, e.g., FIG. 132). Fluid may be delivered through the cartridge 2606 and into the priming line 3120 through the dispensing sharp 2604 of the cartridge 2606 in block 3112. In some examples, a predetermined volume of fluid (based on data from a flow meter) may be delivered through the cartridge 2606 and into the priming line 3120. The flow rate of priming fluid may be controlled to a minimum priming fluid flow rate or a predefined priming fluid flow rate range to assist in ensuring gas is primed out of the cartridge 2606. The priming line 3120 may be displaced out of the cartridge 2606 in block 3114. In block 3116, the prime line 3120 may be grasped by the robotic gripper or manipulator and removed from the retainer on the displacement stage 3142. The prime line 3120 may be returned to a prime line holder or dock in block 3118.

Still referring to FIG. 127, where the robotic gripper is a bag retainer assembly 4306, one or more bag sensor 4312A, B (further described in relation to FIGS. 69A-72A) and/or one or more jaw position sensor 4309 (further described in relation to FIG. 71) may be included in the housing 4314 for the bag retainer assembly 4306. The control system 15 may analyze the output signal from the bag sensor(s) 4312A, B and/or jaw position sensor(s) 4309 to ensure the priming line 3120 is captured in place within the bag retainer assembly 4306 when grasped. The control system 15 may analyze the output signal from the bag sensor(s) 4312A, B and/or jaw position sensors 4309 any time the priming line 3120 is grasped by a bag retainer assembly 4306. The control system 15 may analyze the output signal for the bag sensor(s) 4312A, and/or jaw position sensor(s) 4309 after grasping the priming line 3120, after displacement of the bag retainer assembly 4306 once the priming line 3120 has been grasped or at both of these time points. Likewise, any time a bag retainer assembly 4306 hands off the priming line 3120 to another retainer, the control system 15 may analyze the output signal for the bag sensor(s) 4312A, B after the bag retainer assembly 4306 releases the priming line 3120 (which may be verified by monitoring the output signal from jaw position sensor(s) 4309) and displaces away from the other bag retainer. The control system 15 may verify that the output signal(s) indicate priming line 3120 is no longer present in the bag retainer assembly 4306. In the event that the priming line 3120 is unexpectedly absent or present, a notification may be generated by the control system 15.

Additionally, in examples including an imager type chain orientation sensor 6730 (see, e.g., FIG. 85), the field of view for the imager may be selected to include the priming line 3120 when the priming line 3120 is in any of its expected usage positions. Thus, the chain orientation sensor 6730 may also act as a priming line position sensor. In such embodiments, image data from the chain orientation sensor 6730 may be utilized to detect the priming line 3120 position. The control system 15 may capture an image with the chain orientation sensor 6730 and analyze the image to determine the coordinates of the priming line 3120 within the image. Any suitable approach for detecting the priming line 3120 may be utilized such as any of those described in relation to FIG. 86. The detected coordinates of the priming line 3120 may be checked against an acceptable coordinate range for its expected position. For example, a set of acceptable coordinate ranges may be defined for the priming line 3120 after placement on the fill displacement stage (block 3104), after the priming line 3120 is displaced to each of the disinfect position (block 3016) and ready position (block 3110), and after stowing on the prime line retainer (block 3118). In the event that analysis of the image data by the control system 15 does not detect the priming line 3120 or detects the priming line 3120 in an unexpected or out of range location, a notification may be generated (e.g. indicating user intervention is required). The system 10 may be placed in a standby state until the issue is remedied.

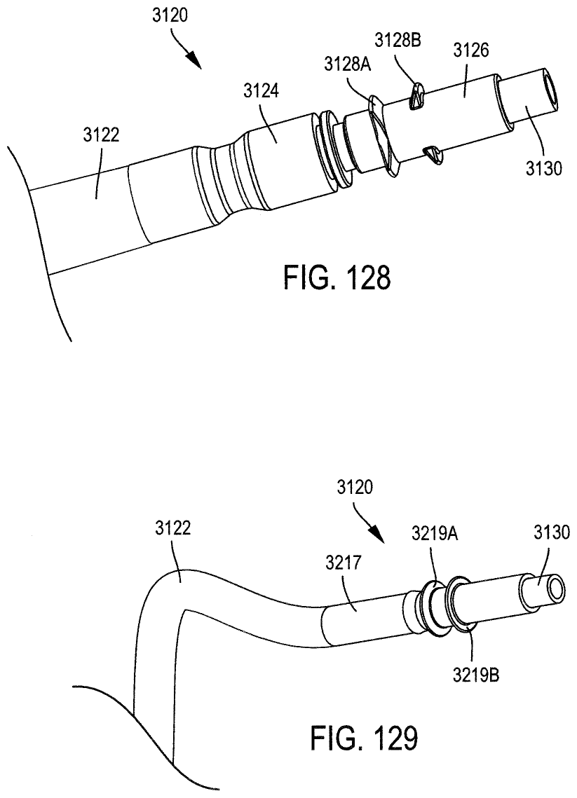

Referring now to FIG. 128, a perspective view of an example prime line 3120 is depicted. As shown, the prime line 3120 may include a section of flexible tubing 3122 which extends from a discard destination (e.g. drain or the input to the water treatment system, see, FIGS. 229A-C). The section of tubing 3122 may be coupled to a prime port hub 3124. A port replica 3126 may be coupled to and extend from the port hub 3124. The port replica 3126 may be shaped to mimic ports 392 of bags 26 for use with the system 10. In the example shown, the port replica 3124 includes retention projections 3128A, B mimicking the projections included on various bag 26 embodiments shown and described herein. The port replica 3126 may be constructed of materials which differ from the port 392. The port replica 3126 may, for instance, be constructed of rigid plastic, ceramic, metal, glass, etc. The terminal end of the prime line 3120 may be formed by a section of tubing 3130. The section of tubing 3130 may be formed of a UVC transparent or at least substantially transmissive material. In some examples, the section of tubing 3130 may be formed of the same material as the barrier 2646 of various cartridges 2606 described herein. Fused quartz or fused silica may be used to form the section of tubing 3130.

An alternative example prime line 3120 is depicted in FIG. 129. In place of a prime port hub 3124 and a port replica 3216, some example prime lines 3120 may include an elongate hollow body 3217 having a spaced set of ribs 3219A, B. The elongate hollow body 3127 may be constructed of rigid plastic, ceramic, metal, glass, etc. The ribs 3219A, B may form rings which project outwardly from the elongate hollow body 3217. The ribs 3219A, B may assist in retention of the prime line 3120 by a holder or automation end effector (see, e.g., bag retention assembly 4306 of FIG. 68B). Including ribs 3219A, B which encircle the elongate hollow body 3217 may allow the prime port 3120 to be grasped without concern for the rotational orientation of the prime port 3120 about the axis of the elongate hollow body 3217. A section of tubing 3130 (e.g. fused quartz) like that described in relation to FIG. 128 may extend from the terminal end of the elongate hollow body 3217 opposite that coupled to the section of tubing 3122.

Referring now to FIG. 130-131, a perspective view of a fill displacement stage 3142 and an example fill station 2600 with a prime line 3120 retained on the fill displacement stage 3142 are respectively depicted. The fill displacement stage 3142 may be part of a bag displacement assembly 3140 including a bag displacement actuator 3144. The fill displacement stage 3142 may be coupled to the bag displacement actuator 3144 via a mounting plate 3148. The fill displacement stage 3142 may include a support plate 3150 which may be shaped to cradle a filled bag 26. The bag displacement actuator 3144 may include an electromagnetic brake which may be engaged when a filled bag 26 is in place on the fill displacement stage 3142. A position sensor 3145 which generates a signal which alters in relation to the position of the fill displacement stage 3142 may be included. For example, a motor encoder may be provided in association with the fill displacement actuator 3144.

Referring primarily to FIG. 130, at least one port retainer 3146 may also be included on the fill displacement stage 3142. In the example embodiment, two port retainers 3146 are depicted though the number of port retainers 3146 may typically match the number of ports 392 on bags 26 used by the system 10. The port retainers 3146 may passively hold ports 392 of a bag 26 which has been placed at the fill station 2600. As the prime line 3120 includes a port replica 3124, the port retainers 3146 may also passively hold the prime line 3120 in place as if it is a bag 26 port 392. Prime lines 3120 with annular ribs 3219A, B may similarly be passively retained. The following description of the fill displacement stage 3142 will refer to ports 392 for sake of brevity, but it should be understood that prime limes 3120 may be held in like manner by port retainers 3146.

In the example, each port retainer 3146 includes a pair of arms 3154A, B which are cradle shaped and able to clip around a port 392 installed in a port retainer 3146. The arms 3154A, B may splay outward from one another as a port 392 is pressed in between their unsupported ends. Thus, the unsupported ends of the arms 3154A, B may form guide regions which assist in directing ports 392 into the port retainers 3146. The distance between the arms 3154A, B adjacent the guides regions may be smaller than the outer diameter of a port 392. The arms 3154A, B may be spread apart and/or a port 392 may be distorted as a port 392 is installed in a port retainer 3146. This may help prevent inadvertent dislodgement of a port 392 from a port retainer 3146 as some force may be required to remove the port 392 from the port retainer 3146. Additionally, the port retainers 3146 may include rest surfaces 3156. In the example embodiment, the rest surfaces 3156 are the top faces of the arms 3154A, B. When installed within a port retainer 3146, a retention projection 140 of a port 392 (see, e.g., FIG. 21E) may abut against the rest surfaces 3156. This may create a mechanical interference which prevents a bag 26 from displacing in a direction parallel to the axial dimension of a port 392. Retention projections 140 may also be present on a component coupled to a port 392. The retention projections 140 may be on projection bearing bodies 142 shown in FIGS. 10A-B, 16A-18C, the ribbing clip 168 of FIG. 13, the ribbed rings 180 of FIGS. 14A-C, or the ribbed spacers 141 shown in FIGS. 15A-B. Ribs 3219A, B of a prime line 3120 may similarly prevent axial displacement of the prime line 3120. In alternative embodiments, one or more active gripper (e.g. a pneumatically powered gripper) may be included on the displacement stage 3142 and may be opened and closed to grasp ports 392 or the prime line 3120.

Referring now primarily to FIGS. 131-132, with the prime line 3120 retained on the fill displacement stage 3142, the prime line 3120 may be displaced into the air flow channel 2802 of a cartridge 2606. Though not shown, the clean air output 3090 (see, e.g., FIG. 114) may be in place and clean air may be flowing through the air flow channel 2802 to achieve the desired level of environmental control in the fill zone volume. Before transferring fluid into the cartridge 2606 to prime the cartridge 2606, the prime line 3120 may be brought to a disinfect position. In the disinfect position the tubing section 3130 may be spaced from the dispensing sharp 2604. The irradiation assembly 2608 may be powered for a predetermined period of time to irradiate the tubing section 3130. The fill displacement stage 3142 may then be actuated to advance the tubing section 3130 over the dispensing sharp 2604 to a cartridge priming position. When the prime line 3120 is in the cartridge priming position, fluid may be delivered to the cartridge 2606 to prime the cartridge 2606. Any priming fluid which exits the cartridge 2606 via the dispensing sharp 2604 may be received by the prime line 3130 and directed to a discard destination through the tubing 3122 of the prime line 3120. In various examples, the control system 15 may command fluid to be delivered to the cartridge 2606 until a flow meter indicates at least a predefined volume of priming fluid has been dispensed to the cartridge 2606. In certain systems 10, the cartridge 2606 may be periodically disinfected with disinfection fluid (e.g. heated water, peracetic acid solution). The prime line 3120 may similarly be positioned in the priming position as disinfection fluid is transferred through the cartridge 2606 and out of the dispensing sharp 2604.

Referring now primarily to FIG. 133, once a cartridge 2606 has been primed, bags 26 may be filled by transferring fluid through the cartridge 2606 into a bag 26. As shown, the prime line 3120 may be withdrawn from the cartridge 2606 and placed on a prime line retainer 3158 once priming is complete. The prime line retainer 3158 may include a port retainer 3146 of the variety described above in relation to FIG. 130. The prime line 3130 may be collected from the fill displacement stage 3142 and stowed in the prime line retainer 3158 by a robotic gripper or manipulator (see, e.g., bag retention assembly 4306 of FIG. 68A).

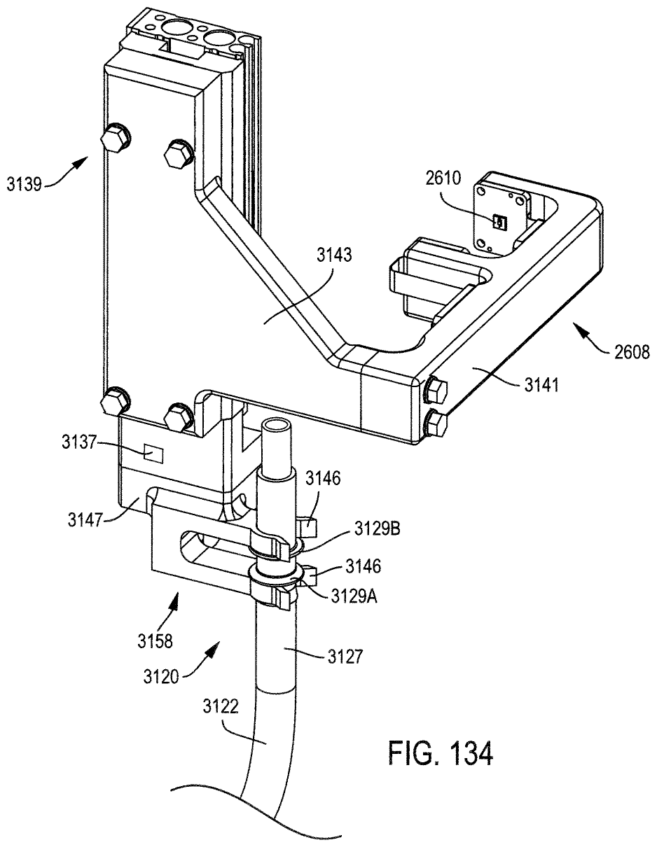

Referring now to FIG. 134, in certain examples, the prime line retainer 3158 may be coupled to the irradiation assembly 2608. As shown, the irradiation assembly 2608 includes an arm 3141 which is coupled to a displacement stage 3143 of the actuation assembly 3139. The prime line retainer 3158 includes a set of port retainers 3146 which extend from a base body 3147 coupled to the displacement stage 3143. As the actuation assembly 3139 is commanded to displace the displacement stage 3143, the prime line retainer 3158 (and prime line 3120 if retained therein) will displace in tandem with the displacement stage 3143. This may, among other benefits, simplify routing and draining of the tubing segment 3122 of the prime port 3120. The port retainers 3146 may be spaced such that the ribs 3129 may be positioned between the port retainers 3146 when the prime line 3120 is retained on the prime line retainer 3158. The spacing between the most proximal faces of the opposing port retainers 3146 may be slightly greater (e.g. 5% or less) than the spacing between the greatest distance between the faces of the ribs 3129A, B. This may inhibit axial displacement of the prime line 3120.

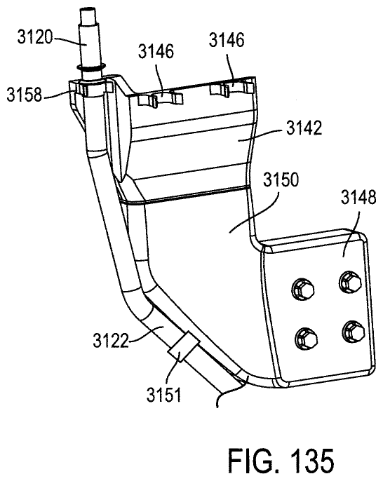

Referring now to FIG. 135, in still other embodiments, the prime line retainer 3158 may be formed as part of the fill displacement stage 3142 of a fill displacement assembly 3140. In some examples, the prime line retainer 3158 may be formed integrally to the fill displacement stage 3142. In such embodiments, the prime line 3120 may displace with the displacement stage 3142. It may also conveniently be ensured to be at even height with the port retainers 3146 on the fill displacement stage 3142. A clip, tether, or constraint member 3151 may be included as part of the fill displacement assembly 3140 to hold or route the tubing 3122 of the prime line 3120 in a desired manner. The constraint member 3151 in the example holds the tubing 3122 adjacent a support plate of the fill displacement assembly 3140. The constraint member 3151 may allow enough displacement of the prime line 3120 that it may be swapped between a port retainer 3146 on the fill displacement stage 3142 and the prime line retainer 3158.

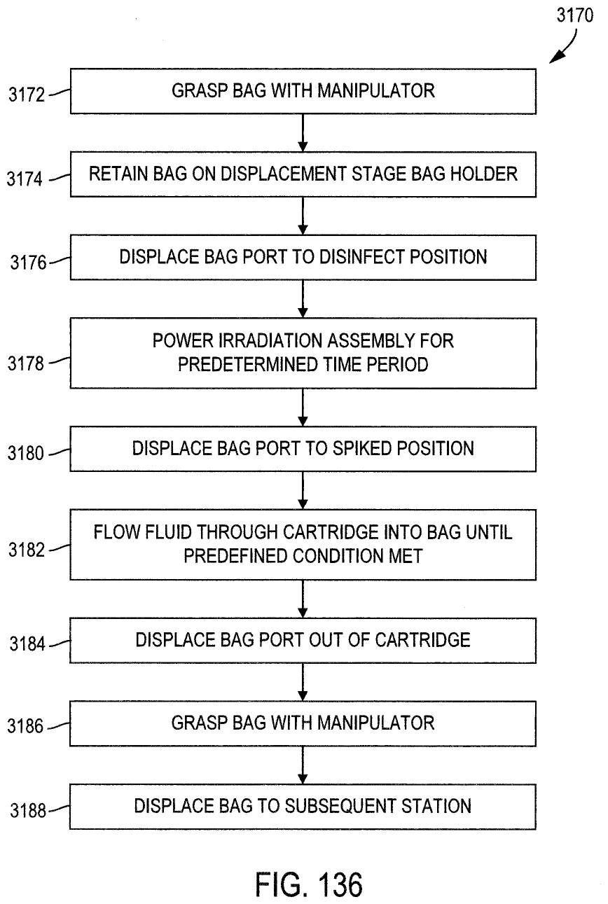

Referring now to FIG. 136, a flowchart 3170 detailing a number of example actions which may be executed to fill a bag 26 is depicted. As shown, in block 3172, a bag 26 may be grasped with a robotic grasper or manipulator (see, e.g., bag retention assembly 4306 of FIG. 68A). The bag 26 may be retained on the fill displacement stage 3142 in block 3174 (see, e.g., FIG. 133). The ports 392 of a bag 26 may, for example, be pressed into port retainers 3146 on the fill displacement stage 3142 to retain the bag 26 on the fill displacement stage 3142. In various embodiments, a singulation assembly 4400 (see, e.g., FIG. 74) may individualize the bag 26 from a chain 2670 of bags 26 (see, e.g., FIGS. 83-86) and associated description) after block 3174.

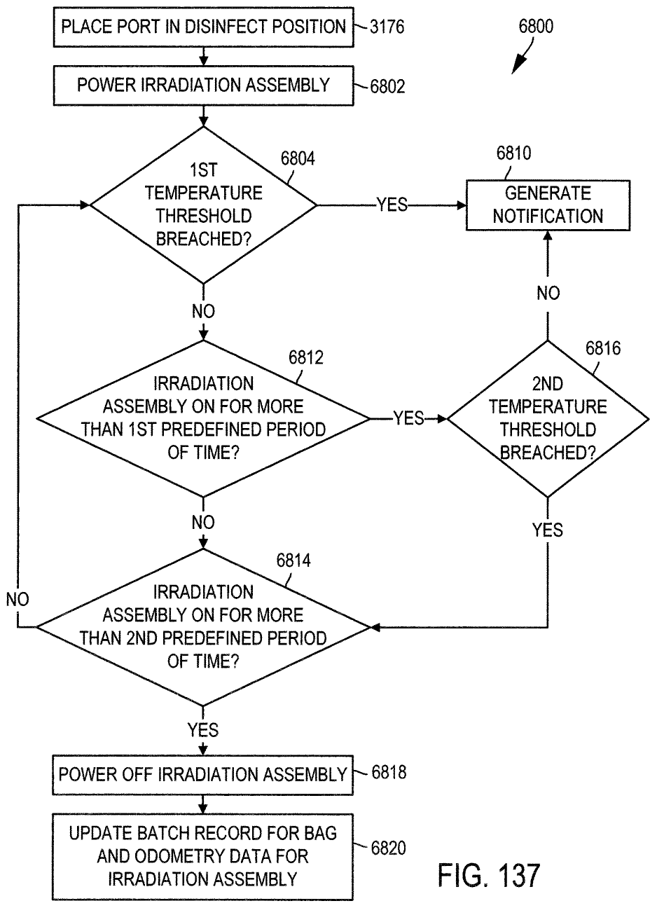

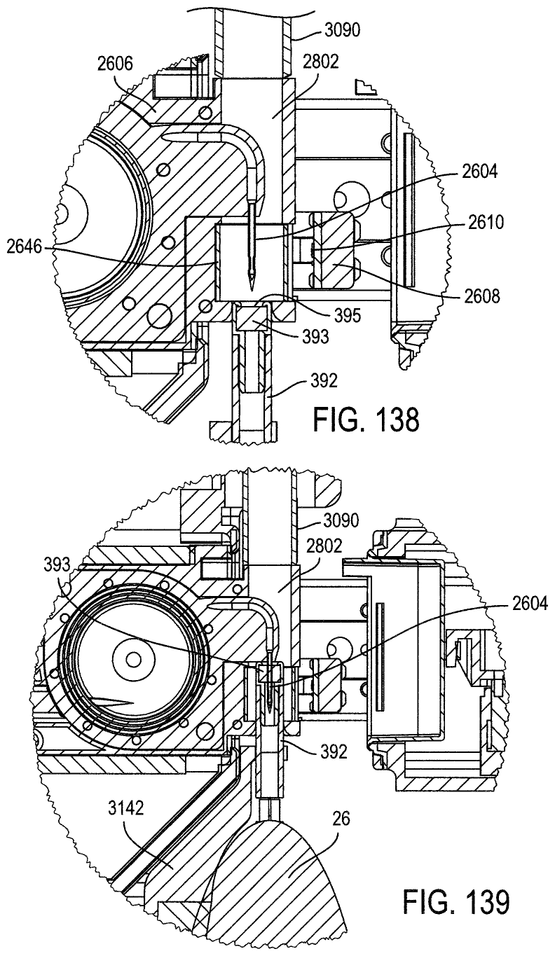

In block 3176, a port 392 of the bag 26 may be displaced to a disinfect position (see, e.g., FIG. 138). The disinfect position may be a position in which the exposed surface 395 of the septum 393 of the bag 26 has been introduced into the air flow channel 2802 and is spaced from the dispensing sharp 2604. The control system 15 may command actuation of the bag displacement actuator 3144 to displace the bag 26 until the port 392 reaches the disinfect position. The irradiation assembly 2608 may be powered for a predetermined period of time in block 3178 (further described in relation to FIG. 137).

The port 392 may be displaced to a spiked position in block 3180 (see, e.g., FIG. 139). Again this may be orchestrated via commands to the bag displacement actuator 3144 generated by the control system 15. Fluid may be delivered into the bag 26 through the dispensing sharp 2604 in block 3182. This may be done until a predefined condition has been met (e.g., the signal from a flow meter indicates a predefined volume of fluid has been dispensed). In block 3184, the port 392 of the bag 26 may be withdrawn from the cartridge 2606. This may be accomplished by commanding actuation of the bag displacement actuator 3144 with the control system 15. The ports 392 of the bag 26 may be grasped by a robotic grasper or manipulator in block 3186. The bag 26 may then be displaced to a subsequent station within the system 10 in block 3188.

Referring now to FIG. 137, a flowchart 6800 is depicted detailing a number of example actions which may be executed to irradiate a port 392 and septum 393 of a bag 26 (or portion of a priming line 3120) with an irradiation assembly 2608. As shown, the port 392 may be placed in a disinfect position in block 3176 (as described in relation to FIG. 136). In block 6802, the irradiation assembly 2608 may be powered. For example, the irradiation assembly 2608 may be powered at 100% duty cycle for 30 seconds. In certain embodiments, the control system 15 may monitor an output signal of at least one temperature sensor on the irradiation assembly 2608 to confirm an over-temperature threshold is not exceeded. In, if in block 6804, the output signal from the temperature sensor(s) indicate that the over-temperature threshold has been exceeded, the control system 15 may generate a notification (e.g. a display screen on the user interface 6100) in block 6810. The system 10 may be placed in a standby state until the issue is remedied (e.g. service personnel indicate service has occurred). The control system 15 may also verify that the irradiation assembly 2608 was successfully turned on. The control system 15 may, for example, verify in block 6816 that the output signal of the temperature sensor(s) indicate the temperature or its derivative breached a predefined threshold after a first preset period of time has elapsed in block 6812. The preset period of time may be, for instance, 10 seconds after the irradiation assembly 2608 has been powered on. A notification may be generated in block 6810 in the event that the temperature threshold is not breached. The system 10 may be placed in standby until the issue is remedied. Once, in block 6814, the irradiation assembly 2608 has been powered for a second preset period of time, the irradiation assembly may be powered off in block 6818. In block 6820, an indication that the irradiation assembly 2608 operated as expected and/or data from the temperature sensor(s) may be written to the batch record for the bag 26 by the control system 15. Additionally, odometry data relating to the irradiation assembly 2608 may be tracked and updated in block 6820. For instance, a total on time for the irradiation assembly 2608 may be tracked and updated. The control system 15 may verify the total on time is below a replacement threshold and generate a notification to service the irradiation assembly 2608 in the event the replacement threshold is exceeded.

Referring now to the progression of FIGS. 133 and 138-139, a bag 26 is shown being displaced from an initial position after being retained by the port retainers 3146 to a spiked position. Though not depicted in FIGS. 133 and 138-139, a clean air outlet 3090 (see, e.g., FIG. 117) may be in place and clean laminar air may be flowing through the air flow channel 2802 of the cartridge 2606 as described elsewhere herein. As shown in FIG. 138, when the bag 26 is displaced by the bag displacement actuator 2144 toward the cartridge 2606, a port 392 of the bag 26 may enter the downstream end of the air flow channel 2802. The exposed portion 395 of the septum 393 in the port 392 may be in the field of illumination of the light emitters 2610 of the irradiation assembly 2608 when the port 392 is in the disinfect position. The port 392 may be illuminated by the irradiation assembly 2608 for a period of time and the port 392 may then be displaced into the dispensing sharp 2604. Fluid may be dispensed through the cartridge 2606 into the bag 26 until the bag 26 is filled as shown in FIG. 139.

The fill station 2606 may continue to fill bags 26 with the installed cartridge 2606 until the cartridge 2606 is to be replaced. The cartridge 2606 may be swapped on a usage based schedule. The control system 15 may, for example, monitor the number of septa 393 pierced by the dispensing sharp 2604 and may require the cartridge 2606 be exchanged after a predefined threshold number of bags 26 have been filled. In some examples, the control system 15 may also require that the cartridge 2606 be exchanged if a predetermined period of time has elapsed since a cartridge 2606 was installed in the fill station 2600. Odometry information in addition to elapsed time may also be utilized. For example, total number of disinfect cycles the cartridge 2606 has been subject to and the total amount of fluid transferred through the cartridge 2606 may trigger the control system 15 to require replacement after respective thresholds for each have been breached. The control system 15 also require the cartridge 2606 be exchanged in the event that certain fault conditions are determined to be present. For example, if an occlusion is detected downstream of the supply manifold 2820 (see, e.g., FIG. 126A), the control system 15 may require the existing cartridge 2606 be replaced. The control system 15 may receive flow rate and pressure data from sensors monitoring the flow of fluid to the cartridge 2606. The control system 15 may generate an alert and/or require a filter be replaced in the event that analysis of this data indicates that the filter 2609 has failed or become fouled. A failed filter 2609 may be determined in the event that a target flow rate may be achieved at a pressure which is below an expected threshold. A fouled filter 2609 may be detected in the event that pressure rises above a threshold or when the pressure required to maintain a target flow rate exceeds an expected threshold. The control system 15 may also require replacement of the cartridge 2606 in the event that the environment within the processing chamber 4300 does not conform to quality standards or is accessed by service personnel. In some examples, the current draw of an electromechanical actuator for the fill displacement stage 3142 may be monitored as the septum 393 for each bag 26 is spiked by the dispensing sharp 2604 of the cartridge 2606. In the event the motor torque indicated by the current draw is in excess of a predefined threshold or is in excess of the threshold for a number of septa 393 pierces, the control system 15 may require replacement of the cartridge 2606.

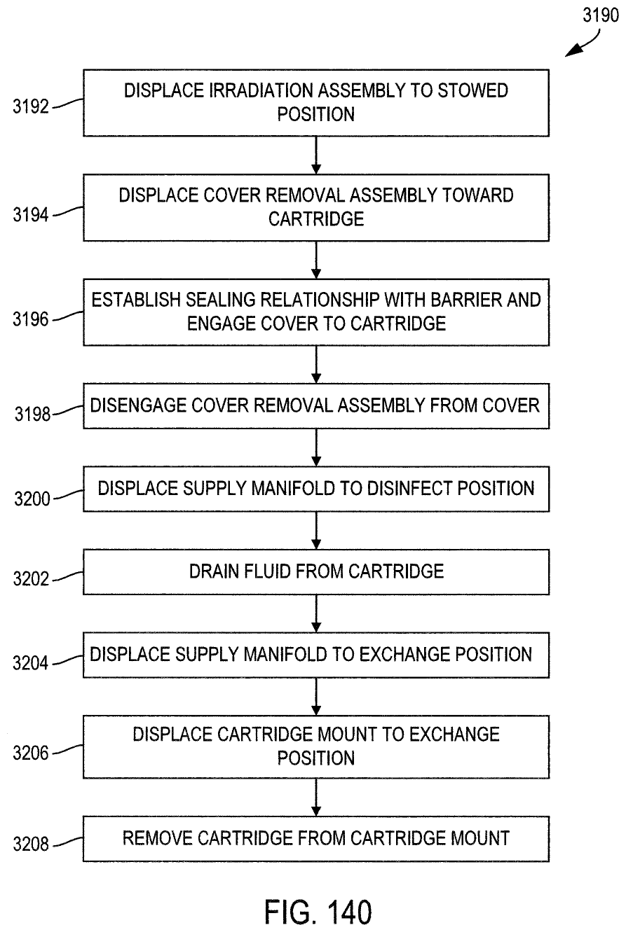

Referring now to FIG. 140, a flowchart 3190 detailing a number of example actions which may be executed to extract a cartridge 2606 from a fill station 2600 is depicted. As shown, the irradiation assembly 2608 may be displaced to a stowed position in block 3192. The housing 3052 of the cover removal assembly 3050 may be displaced toward the cartridge 2606 in block 3194. In block 3196, a sealing relationship may be established against enclosure 12 with the sealing member 3066 of the housing 3052. The cover 2992 may additionally be reengaged to and form a seal against the cartridge 2606 in block 3196. The cover removal assembly 3050 may be disengaged from the cover 2992 in block 3198. In block 3200, the supply manifold 2820 may be displaced to the disinfect position. The cartridge 2606 may be drained in block 3202. This may be accomplished by powering a pump in communication with a fluid line leading from the disinfect outlet channel 2824 of the supply manifold 2820. The supply manifold 2820 may be displaced such that the connection port 2812 is in the partially installed position in the receptacle 2818 for this purpose. In certain examples, the cartridge 2606 may not be drained. This may depend on the type of filter 2609 (if any) included in the cartridge 2606. The supply manifold 2820 may be displaced to a stowed or exchange position in block 3204. In block 3206, the cartridge mount 3030 may be displaced to the exchange position. The cartridge 2606 may be removed from the cartridge mount 3030 in block 3208.

As mentioned in relation to FIG. 89, in certain examples, the entire dispensing assembly 2602 may be a durable component with no removable cartridge 2606. An example of a fill station 2600 with a dispensing assembly 2602 that is replaced as a single unit is shown in FIG. 141. The dispensing assembly 2602 may be directly plumbed to a fluid source 2810 (see, e.g., FIG. 90) and the irradiation assembly 2608 as well as clean air output 3090 may be integrated into the dispensing assembly 2602. This may allow a number of moving components to be omitted from a system 10. For example, a cartridge displacement assembly 3032 (see, e.g., FIG. 105), a cover removal assembly 3050 (see, e.g., FIG. 108A), air output actuation assembly 3094 (see, e.g., FIG. 116), irradiation assembly actuation assembly 2821 (see, e.g., FIG. 125), supply manifold 2820 and accompanying manifold displacement assembly (see, e.g., FIG. 107) may be omitted. A filter assembly 2838 may be included in the flow path 7600 leading from the fluid source 2810 to the dispensing assembly 2602. The prime line 3120 may be brought to the dispensing assembly 2602 and used for priming and or disinfecting the dispensing assembly 2602 as described elsewhere herein. Likewise, ports 392 of bags 26 may be displaced into the dispensing assembly 2602, disinfected, spiked, and filled, as described elsewhere herein.

Referring now also to FIGS. 142A-C, the cartridge 2606 portion of the dispensing assembly 2602 which surrounds the dispensing sharp 2604 may be attached to a base plate 7602. The base plate 7602 may have a cross-sectional area which increases (in the example embodiment in a stepwise fashion) as distance from the cartridge 2606 increases. The enclosure 12 may include a fenestration 3074. The smaller cross-section tier 7606A of the base plate 7602 may be shaped complimentarily to the fenestration 3074 and snuggly fit within the fenestration 3074. A sealing member (e.g. O-ring, not shown) may be included in a groove on the sidewall of the smaller cross-section tier 7606A and compress against the sidewall of the fenestration 3074 when the dispensing assembly 2602 is in place. The larger cross-section tier 7606B may be coupled (e.g. fastened) to the exterior wall of the enclosure 12 surrounding the fenestration 3074. The fenestration 3074 may be sized to allow the cartridge 2606 portion of the dispensing assembly 2602 to fit through the fenestration 3074 when the dispensing assembly 2602 is tilted.

In some embodiments, dispensing assembly 2602 may be coupled to a panel 7601 which is itself removable. This panel 7601 may be decoupled from the remainder of the enclosure 12 during service to allow a gowned individual access to the processing compartment 4300 for service or cleaning.

The smaller cross-section tier 7606A of the base plate 7602 may include a stage 7604 to which the cartridge 2606 may be coupled (e.g. via fasteners). A sealing member 7608 may be compressed between the stage 7604 and the cartridge 2606 when the two are coupled together. The cartridge 2606 may include a clean air output 3090, fluid inlet portion 7610, irradiation assembly housing 7612, and an end cap portion 7614. At least one sealing member 7616 may be disposed and compressed intermediate the clean air output 3090 and the fluid inlet portion 7610. At least one sealing member 7618 may be disposed and compressed between the fluid inlet portion 7610 and the irradiation assembly housing 7612. At least one sealing member 7620 may also be disposed and compressed between the irradiation assembly housing 7612 and the end cap portion 7614. An air flow channel 2802 may extend through each of the clean air output 3090, fluid inlet portion 7610, irradiation assembly housing 7612, and end cap portion 7614. The shape of the bore 3097 may be as described above in relation to FIGS. 115A-B. The portion of the air flow channel 2802 extending through the remainder of the cartridge 2606 may have a substantially constant diameter. Though not shown, the end cap portion 7614 may include guides 2658 (see, e.g., FIG. 101B) which may assist in constraining a port 392 of a bag 26 in a prescribed orientation relative to the cartridge 2606 when the port 392 is introduced to the air flow channel 2802.

Referring now primarily to FIGS. 142B-C, the fluid inlet portion 7610 may include a hub 2640 to which the dispensing sharp 2604 of the dispensing assembly 2602 is coupled. An inflow passage 7622 may extend though the hub 2640 to the dispensing sharp 2604 from a fitting 7624 for attachment to a conduit forming the flow path 7600 to the fluid source 2810. As shown, the fluid inlet portion 7610 includes a threaded fitting 7626 which may mate with a corresponding threaded region of the clean air output 3090 to couple the clean air output 3090 to the fluid inlet portion 7610. In alternative embodiments, the clean air output 3090 may be coupled to the fluid inlet portion 7610 in any other suitable manner. The portion of the airflow channel 2802 defined by the clean air output 3090 may be of variable cross-sectional area as shown and described in relation to FIG. 115B.

The irradiation assembly housing 7612 (best shown in FIG. 142B) may house the irradiation assembly 2608 of the dispensing assembly 2602. As shown, a number of antimicrobial light emitters 2610 may be included in the irradiation assembly 2608. The antimicrobial light emitters 2610 may be positioned to at least partially surround a barrier 2646 of UVC translucent or transparent material. The barrier 2646 may be captured within the cartridge 2606 between the end cap portion 7614 and a ledge of the irradiation assembly housing 7612. The inner wall of the barrier 2646 may form a portion of the wall of the air flow channel 2802. As shown, the entirety of the airflow channel 2802 may be formed by the clean air output 3090, fluid inlet portion 7610, irradiation assembly housing 7612, barrier 2646, and end cap portion 7614. None of the sealing members 7616, 7618, 7620 may be in communication with the air flow channel 2802. This may help ensure the wall of the air flow channel 2802 is free of bumps (e.g. due to bulging of compressed sealing members 7616, 7618, 7620).

The antimicrobial light emitters 2610 may be mounted on respective rigid portions 7628 of a rigid-flex PCB 7630. The rigid-flex PCB 7630 may routed though a cavity between the barrier 2646 and an internal wall of the irradiation assembly housing 7612 surrounding the barrier 2646. Preferably the rigid portions 7628 of the rigid-flex PCB 7630 may be coupled to the irradiation assembly housing 7612 with a thermal adhesive 7632. The irradiation assembly housing 7612 may also preferably be constructed of a material having a thermal conductivity at least equal to that of aluminum (and may be aluminum). The rigid-flex PCB 7630 and antimicrobial light emitters 2610 may be isolated from the volume of the air flow channel 2802 by the barrier 2646 shielding them from contact with various agents which may be present in the air flow channel (e.g. VHP during cleaning).

Aside from allowing removal of a cartridge displacement assembly 3032 (see, e.g., FIG. 105), cover removal assembly 3050 (see, e.g., FIG. 108A), air output actuation assembly 3094 (see, e.g., FIG. 116), irradiation assembly actuation assembly 2821 (see, e.g., FIG. 125), supply manifold 2820 and accompanying manifold displacement assembly (see, e.g., FIG. 107), a dispensing assembly 2602 of the variety shown in FIGS. 141-142C may provide additional advantages. For example, the clean air output 3090 may be fixed in direct engagement with the air flow channel 2802 in the cartridge 2606. The input end of the clean air output 3090 may also be disposed in fixed relation to the controlled environment supply plenum 6228 (see, e.g., FIG. 248) of an air handling assembly 6200 (see, e.g., FIG. 244). Thus, a direct pathway from the controlled environment supply plenum 6228 through the air flow channel 2802 of the dispensing assembly 2602 may consistently be present after a dispensing assembly 2602 is installed. This may simplify air handling in the processing chamber 4300 of the system 10. The bulk of processing chamber 4300 may be kept at a first level of environmental control stringency while the air flow channel 2802 may be kept at a second, higher level of environmental control stringency. Use of a dispensing assembly 2602 of the variety shown and described in relation to FIGS. 141-142C may avoid a transitory period where the entire processing chamber 4300 is brought to the second level of stringency. This may be done in certain embodiments where the air flow channel 2802 is out of engagement with the clean air output 3090 for periods of time. This may, for instance, occur in some embodiments when a disposable cartridge 2606 is first installed and the cover 2992 (see, e.g., FIG. 112) is removed by a cover removal assembly 3050 (see, e.g., FIG. 108A).

Still referring to FIGS. 141-142C, during use of the shown dispensing assembly 2602 various odometry data may be collected. Any of the odometry data described above in relation to various disposable cartridges 2606 may be tracked and included in batch records for bags 26. Additionally, any odometry data related to the irradiation assembly 2608 described above may similarly be tracked and included in batch records.

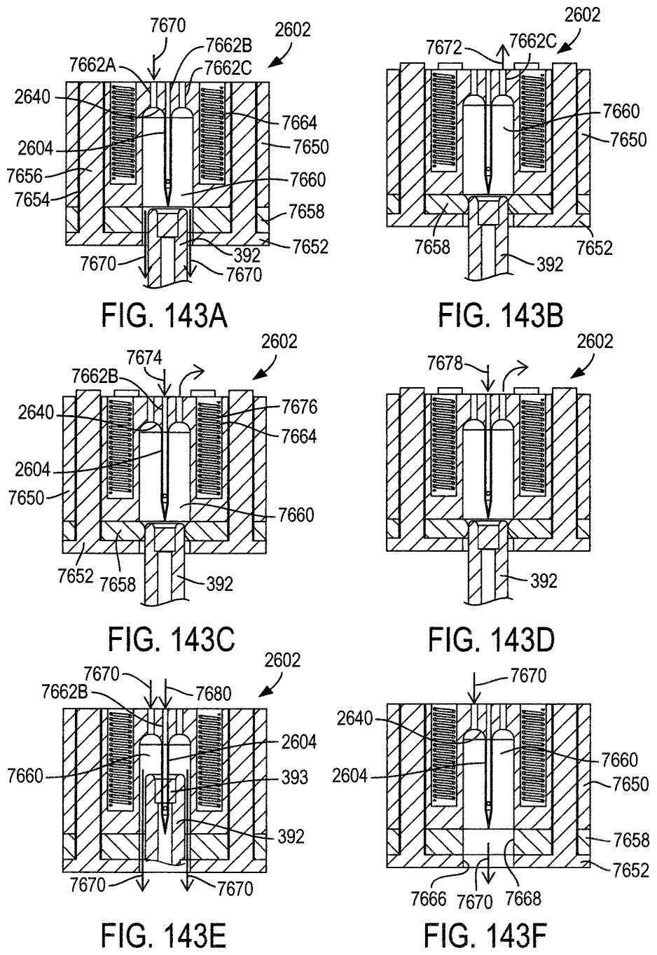

Referring now to FIGS. 143A-F, another exemplary dispensing assembly 2602 is depicted. The example dispensing assembly 2602 may include a housing 7650. The housing 7650 may include a receptacle 7660 having an open end and an opposing end with a number of fluid transfer ports 7662A-C. The opposing end is shown as closed in the example embodiment though it should be understood that the example dispensing assemblies 2602 depicted in FIGS. 143A-F are schematic representations. In alternative embodiments, the opposing end may be open and the hub 2640 may be disposed intermediate the two ends of the receptacle 7660 (see, e.g., FIG. 103C). In such examples, an end of the receptacle 7660 upstream of the hub 2640 may act as a fluid transfer port 7662A and may engage with or couple to a clean air output 3090 (see, e.g., FIG. 118 and FIG. 142A). Other fluid transfer ports 7662B-C may be routed through the sidewall of the receptacle 7660 in such examples.

A hub 2640 may also project into the receptacle 7660 at the closed end. A dispensing sharp 2604 may be coupled to the hub 2640 and be in communication with one of the fluid transfer ports 7662B though the hub 2640. The housing 7650 may also include a plurality of pockets 7664 in a wall of the housing 7650 surrounding the receptacle 7660. In alternative embodiments, the pockets 7664 may be joined into a single continuous depression which surrounds the receptacle 7660.

The dispensing assembly 2602 may also include an end cap 7652. The end cap 7652 may be disposed at the open end of the housing 7650. The end cap 7652 may include an aperture 7666 aligned with the receptacle 7660 which extends through the end cap 7652. The end cap 7652 may be displaceable relative to the housing 7650. The end cap 7652 may, for example, be coupled to a linear actuator (not shown) which may drive the end cap 7652 toward and away from the housing 7650. The housing 7650 may include tracks 7654 through which guide portions 7656 of the end cap 7652 extend. This may constrain displacement of the end cap 7652 relative to the housing 7650 along a prescribed path.

A compliant body 7658 may be disposed intermediate the housing 7650 and the end cap 7652. The compliant body 7658 may be constructed of a soft durometer elastomer (e.g. 80 durometer tetrafluoroethylene/propylene copolymer or FEPM). The compliant body 7658 may include an aperture 7668 aligned with the receptacle 7660. The end cap 7652 may be displaced from a home position (see, e.g., FIG. 143F) towards the housing 7652 to a compressing position (see, e.g., FIG. 143B). As this occurs, the compliant body 7658 may compress and material may be forced toward the axis of the receptacle 7660. This may decrease the diameter of the aperture 7668 in the compliant body 7658.

Still referring to FIGS. 143A-F, bags 26, which have been previously sterilized, may be filled in a locally established or miniaturized clean room like environment provided by the dispensing assembly 2602. This environment or fill zone may be present in a small space (e.g. 100 ml or less in volume) within the dispensing assembly 2602. The fill zone may also be placed into open communication with other less stringently controlled zones. A fill zone may be sized to accept a portion of a bag 26, for example, a port 392 or end portion of a port 392. The fill zone volume may for example conform to or exceed particle requirements, as well as appropriate relative pressure, air exchange rate, and/or air velocity set points for a selected cleanroom level (e.g. ISO 5 clean room set points). Thus, the receptacle 7660 may be held to a stringent level of environmental control. This level may, moreover, be more stringent than that imposed on the interior volume of the processing chamber 4300 with which the receptacle 7660 communicates. Thus, a small fill zone volume which is under relatively strict environmental control may be established. The small fill zone volume of the example dispensing assembly 2602 may also be selectively isolated from the surrounding and potentially less stringently controlled environment. When isolated, the small fill zone may be utilized as a miniaturized flash sterilization chamber.

Referring now primarily to FIGS. 143A-B, to fill a bag 26, a port 392 of the bag 26 may be introduced into the dispensing assembly 2602 through the apertures 7666, 7668 of the end cap 7652 and compliant body 7658. Clean air 7670 may be passed through the receptacle 7660 as the port 392 is introduced. The port 392 may be advanced into the dispensing assembly 2602 to a disinfecting position where the port 392 is in spaced relation to the tip of the dispensing sharp 2604. The end cap 7652 may then be displaced to the compressing position and forcing the compliant body 7658 to bulge toward the axis of the receptacle 7660. This may cause a radial seal to form against the exterior surface of the port 392. The flow of clean air 7670 may be cut and a vacuum 7672 may optionally be drawn on the receptacle 7660 by placing a fluid transfer port 7662C in communication with a vacuum source (e.g. by actuation of one or more valve).

Referring now to FIG. 143C, the fluid transfer port 7662B in fluid communication with the dispensing sharp 2604 via the hub 2640 may be connected to a clean steam source and first clean stream 7674 may be delivered through the dispensing sharp 2604 to the receptacle 7660. Other fluid transfer ports 7662A, C may also be placed into communication with the clean stream source as well. The first clean steam 7674 may be saturated steam at 132° and 28 psig in certain examples. The first clean steam 7674 may have a dryness factor of at least 0.8 and more preferably 0.95. The steam may be generated from the product output by a fluid handling system 3900 (see, e.g., FIGS. 229A-C). Alternatively, steam may be sourced from a vapor compression distillation device included in the fluid handling system 3900. Example vapor compression distillation devices include any of those shown and described in U.S. Pre-Grant Publication No. US20210395109A1, filed Mar. 29, 2021, and entitled Water Vapor Distillation Apparatus, Method, and System (Atty, Docket No. AA542) which is hereby incorporated by reference herein in its entirety. Where a vapor compression distillation device is used, pressure regulators, heaters, etc. may be included to assist in conditioning the steam to desired set points. The first clean steam 7674 may disinfect the dispensing sharp 2604, receptacle 7660, and port 392. First clean steam 7674 may be exhausted out of a fluid transfer port 7662C in fluid communication with the receptacle 7660. In some examples, a small vacuum may be applied through the fluid transfer port 7662C to assist in exhausting the first clean stream 7674.

The pockets 7664 in the housing 7650 may also be supplied steam and serve as a steam jacket for the receptacle 7660. As shown, the pockets 7664 include heating coils 7676. The heating coils 7676 may be powered to heat the steam in the pockets 7664 and maintain the steam jacket. The receptacle 7660 may be subjected to the first clean steam 7674 for a first predefined period of time (e.g. at least 3 min.). Alternatively, no steam jacket may used and the housing 7650 may be heated by heating coils 7676.

Referring now to FIG. 143D, a second clean steam 7678 may be dispensed through the fluid transfer port 7662B and out of the dispensing sharp 2604 to the receptacle 7660. The second clean stream 7678 may also be supplied through other fluid transfer ports 7662A, C. The second clean steam 7678 may be superheated. Any condensate resulting from application of the first clean steam 7674 may be removed from the receptacle 7660 by re-evaporation or mechanical entrainment. The second clean steam 7678 may be supplied for a second predefined period of time. The first and second steams 7674, 7678 may be provided from different source supplies. Alternatively, the first and second steam 7674, 7678 supplies may be from a single supply source. The second steam may be route to the dispensing assembly 2602 through a different pathway which may for example include a heater.

Referring now primarily to FIG. 143E, after disinfection and drying, the end cap 7652 may be displaced to the home position and the radial seal against the exterior of the port 392 may be removed. Clean air 7670 may be delivered through the receptacle 7660 and the port 392 may be displaced relative to the dispensing assembly 2602 to pierce the dispensing sharp 2604 through a septum 393 of the port 392. A filling liquid 7680 (e.g. WFI) may be delivered through the fluid transfer port 7662B to the dispensing sharp 2604 to fill the bag 26. Once a target amount of filling liquid has been dispensed, the flow of filling liquid 7680 may be halted and the port 392 may be removed from the dispensing assembly 2602. Clean air 7670 may continue to be delivered through the receptacle 7660 to keep the receptacle 7660 at a more stringent level of environmental control than the surround portions of the processing chamber 4300.

Referring now to FIGS. 144A-C, once a bag 26 has been filled at a filling station 2600 it may be retrieved from the filling station 2600 and displaced through the processing chamber 4300 to a transfer chamber 3506 of the system 10. A top plan view of a processing chamber 4300 and transfer chamber 3506 are depicted in FIG. 144A. A filled bag 26 is shown in place at the filling station 2600 in FIGS. 144B-C (the chain 2670 is hidden). A bag 26 may be collected by the downstream displacement assembly 4304 after filling. The bag retention assembly 4306 of the downstream displacement assembly 4304 may be extended toward the fill displacement stage 3142. The jaws 4308A, B of the bag retention assembly 4306 may be closed around the ports 392 of the filled bag 26. At least one set of ribs 140 on the port 392 may be disposed above the jaws 4308A, B of the bag retention assembly 4306 (best shown in FIG. 144C). The bag retention assembly 4306 may then be displaced at least partially toward the retracted position. This may dislodge the ports 392 of the filled bag 26 from the port retainers 3146 on the fill displacement stage 3142. The ribs 140 (or other protrusions) on the ports 392 may assist in ensuring the filled bag 26 is robustly held by the bag retention assembly 4306. The ribs 140 may inhibit the bag 26 from falling through the bag retention assembly 4306 and may allow the bag retention assembly 4306 to only lightly grip the ports 392 despite the weight of the filled bag 26. This may allow greater latitude in selection of actuators for the jaws 4308A, B.

Referring primarily to FIGS. 144B-C, the doors 4700, 4702 of the transfer chamber 3506 may make a fluid tight seal against walls of the transfer chamber 3506. Each door 4700, 4702 may include a sealing member 4708 near a peripheral edge of the face 4714 of the door 4700, 4702 most proximal the wall against which it is to seal. The walls of the transfer chamber 3506 may optionally have a rib 4710 surrounding the fenestration 4712 in the wall associated with the door 4700, 4702. Each door 4700, 4702 may be pivotally displaceable. The doors 4700, 4702 may each be coupled to a respective rotary door actuator 4716 which may be powered by the control system 15 to displace the coupled door 4700, 4702. In the example, the rotary door actuators 4716 are disposed outside of the transfer chamber 3506 and transmit rotation to the doors 4700, 4702 via a sealed pass-through in the wall of the transfer chamber 3506. Preferably, the doors 4700, 4702 and associated fenestrations 4712 in the transfer chamber 3506 are kept as small as is practicable. Fenestrations 4712 may be sized to accept a bag retention assembly 4306 holding a filled bag 26 with the bag retention assembly 4306 in a fully retracted state.

As mentioned elsewhere herein, the control system 15 may prohibit both doors 4700, 4702 being in an open state at the same time. When one door 4700, 4702 is opened, the other door 4700, 4702 would be in a closed state. After a door 4700, 4702 is transitioned to a closed state, a recovery period may be imposed by the control system 15 before another door 4700, 4702 is permitted to be opened. In some examples, the recovery period may only be present (or may be longer) when the door 4700 to the processing compartment 4300 is to be opened after the door 4702 to the outfeed compartment 4202 has been closed. The recovery period may be defined by one or more of a preset number of air exchanges or a preset period of time. A predefined pressure may also be required to be maintained in the transfer chamber 3506 during the recovery period. In various embodiments, requirements related to any other environmental control characteristic mentioned herein may also be imposed on the recovery period.

Referring now to FIG. 145, the door 4700 to the transfer chamber 3506 may be actuated to an open position and the downstream displacement assembly 4304 may be driven at least partially into the transfer chamber 3506. The bag retention assembly 4306 may be in a fully retracted state as it is displaced through the fenestration 4712. The door 4700 may be opened relatively slowly so as to mitigate perturbation of airflow through the processing compartment 4300. The door 4700 may, for example, be opened 30-50° per second or slower.

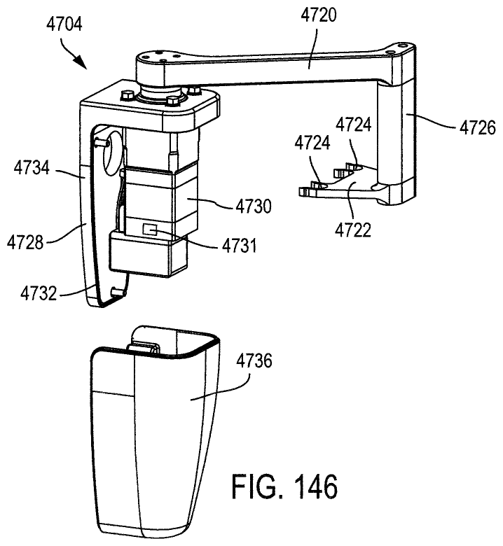

Referring now also to FIG. 146, a bag retainer 4704 may be disposed in the transfer chamber 3506. The bag retainer 4704 may include an arm 4720 to which a retention plate 4722 is coupled. The retention plate 4722 may include a set of port retainers 4724. The port retainers 4724 may be the same as the port retainers 3146 on the fill displacement stage 3142 (see, e.g., FIG. 130). In the example shown in FIG. 146, the retention plate 4722 is coupled to the arm 4720 by a standoff 4726 which disposes the port retainers 4724 slightly above or below the jaws 4308A, B of the bag retention assembly 4306 of the downstream displacement assembly 4304. Though the bag retainer 4704 is shown as including passive retention features for holding a bag 26 in alternative embodiments an actuatable gripper may be included. A pneumatically actuated set of jaws 4308A, B similar to those included in the bag displacement assembly 2618 (see, e.g., FIG. 68A) may, for example, be included. In various examples, the port retainers 4724 may be associated with at least one bag sensor 4312A, B such as any of those shown or described herein (see, e.g., FIG. 68B). Jaw position sensor(s) 4309 (see, e.g., FIG. 71) may also be included.

The arm 4720 is operatively coupled with a rotary actuator 4730. The rotary actuator 4730 may include a sensor 4731 such as a motor encoder or the like which is in data communication with the control system 15. The control system 15 may generate commands to the rotary actuator 4730 based on data from the motor encoder to drive the arm 4720 and retention plate 4722 to a desired position. The rotary actuator 4730 may include a motor brake which may be engaged when the arm 4720 has been positioned as desired. As shown, the rotary actuator 4730 may be enclosed within a housing 4728 (shown exploded apart in FIG. 146). The housing 4728 may include a base 4734 and a cover 4736. A sealing member 4732 such as an o-ring or gasket may be included to ensure the interface between the base 4734 and cover 4736 is airtight. In FIG. 146, the sealing member 4732 is coupled to the base 4734.

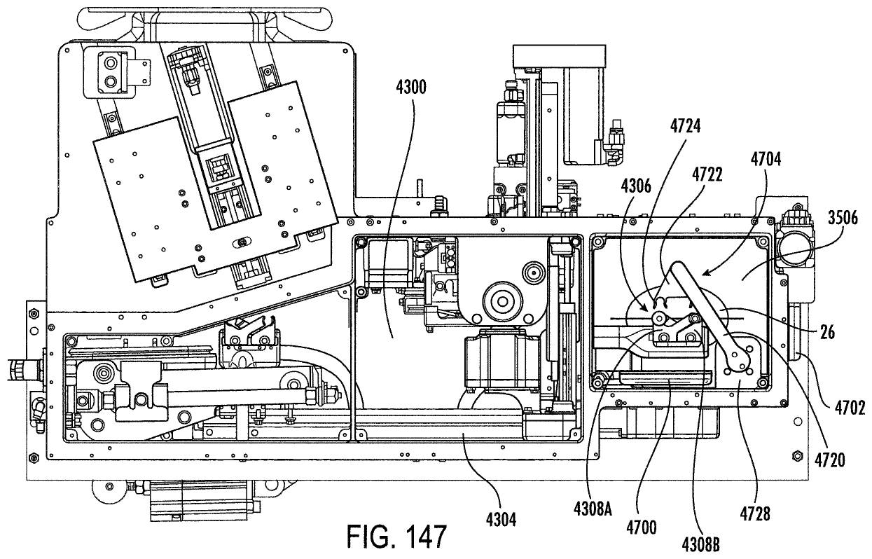

Referring now also to FIG. 147, the rotary actuator 4730 of the bag retainer 4704 may be powered to drive the retention plate 4722 to a first handoff position. In the first handoff position, the bag retainers 4724 of the retention plate 4722 are aligned with the ports 392 of the bag 26 held by the bag retention assembly 4306 of the downstream displacement assembly 4304. The bag retention assembly 4306 of the downstream displacement assembly 4304 may then be displaced to an extended state as shown in FIG. 148. This may press the ports 392 of the filled bag 26 into engagement with the port retainers 4724 of the retention plate 4722. The bag retention assembly 4306 may then be retracted away from the retention plate 4722. At least one set of ribs 140 on each port 392 may be above the port retainers 4724 of the retention plate 4722 to assist in ensuring that the filled bag 26 will not slip through the port retainers 4724.

Referring now to FIG. 149, once the filled bag 26 has been handed off to the bag retainer 4704, the downstream displacement assembly 4304 may be driven out of the transfer chamber 3506. At least one bag sensor 4312A, B (see, e.g., FIG. 68B) included in the bag retention assembly 4306 may be monitored by the control system 15 to verify no bag 26 is present in the bag retention assembly 4306. Alternatively or additionally, where at least one bag sensor 4312A, B is included in the bag retainer 4704, the control system 15 may also monitor the output of any such sensor(s) to verify the port retainers 4724 are occupied by ports of a bag 26. In the event a bag 26 is determined to be unexpectedly present or absent in the bag retention assembly 4306 or bag retainer 4704, a notification (e.g. screen on a user interface 6100) may be generated and the system 10 may be placed in a standby state. In some examples, additional attempted exchanges (up to a predefined cap) may be orchestrated by the control system 15 in the event the bag 26 is determined to remain in the bag retention assembly 4306.

With the bag 26 transferred to the bag retainer 4704, the bag retainer 4704 may be displaced to a second handoff position. The door 4700 between the transfer chamber 3506 and processing chamber 4300 may be actuated closed. The door 4702 between the transfer chamber 3506 and outfeed compartment 4202 may then be opened (optionally after a recovery condition is met). A gripper assembly 4204 of the gantry assembly 4200 may be introduced through the fenestration 4712 associated with the door 4702. Jaws 4205A, B of the gripper assembly 4204 may be closed about the ports 392 of the bag 26 as shown in FIG. 150. The gantry assembly 4200 may then be commanded by the control system 15 to displace the filled bag 26 out of the port retainers 4724 and extract the bag 26 from the transfer chamber 3506 as shown in FIG. 151. At least one of the sets of ribs 140 on each port 392 may be disposed above the jaws 4205A, B of the gantry gripper 4204. This may assist in ensuring the bag 26 is robustly held even if loosely gripped. The door 4702 may then be actuated closed to isolate the transfer chamber 3506. The control system 15 may ensure a recovery period or a recovery condition has been met before opening the door 4700 to receive a next bag 26 from the processing chamber 4300.

In some examples, before the door 4702 is closed, the control system 15 may verify data from at least one sensor indicates the handoff to the gantry gripper 4204 was successful. At least one bag sensor 4312A, B (see, e.g., FIG. 68B) included in the bag retainer 4704 may be monitored by the control system 15 to verify no bag 26 is present in the bag retainer 4704. Alternatively or additionally, where at least one bag sensor 4312A, B is included in the gantry gripper 4204, the control system 15 may also monitor the output of any such sensor to verify the ports 392 are present in the gantry gripper 4204. In some examples, the at least one bag sensor 4312A, B of the gantry gripper 4204 may monitor the position of the jaws of the gantry gripper 4204. The control system 15 may determine a bag 26 is held by the gantry gripper 4204 in the event that the jaws are within a range of, but not at a fully closed position after they are actuated to a closed state. In the event a bag 26 is determined to be unexpectedly present or absent in the bag retainer 4704 or gantry gripper 4702, a notification (e.g. screen on a user interface 6100) may be generated and the system 10 may be placed in a standby state. In some examples, additional attempted exchanges (up to a predefined cap) may be orchestrated by the control system 15 in the event the bag 26 is determined to remain in the bag retainer 4704.

The batch record for the bag 26 may be updated each time an exchange between retainers and/or graspers is completed (or determined to have failed). The batch record may for example be updated by the control system 15 to indicate that outcome of the exchange (e.g. pass/fail) and potential number of retries attempted (if any).

Referring now to FIGS. 152A-C, an exemplary mix assisting assembly 2500 and bag 26 are depicted. Mix assisting assemblies 2500 may be included in systems 10 where the bags 26 include a concentrate. The concentrate may be a solid (e.g. powder, crystalline concentrate, lyophilized medicament, etc.) or liquid (e.g. concentrated medical solution, brine, etc.). In certain implementations, a mix assisting assembly 2500 may be included in systems 10 which includes a bag 26 with a plurality of internal chambers which are user or machine interruptible, though use with bags 26 having a single, continuous interior volume is also possible. Example mix assisting assemblies 2500 may act upon an exterior of a bag 26 to encourage mixing the contents of the bag 26. Alternatively or additionally, mix assisting assemblies 2500 may also displace an entire bag 26 to encourage mixing of the contents of the bag 26. In certain embodiments, an example mix assisting assembly 2500 may be operated until a solution formed in a bag 26 has substantially uniform characteristics throughout the bag 26. Mix assisting assemblies 2500 may, for example, mix concentrate with fluid, excipient, or diluent (e.g. Water for Injection) dispensed into a bag 26. In embodiments where the concentrate is a solid (e.g. crystalline salt), the mix assisting assembly 2500 may also encourage dissolution of the concentrate into fluid dispensed into the bag 26. A mix assisting assembly 2500 may further cause any particulate within a bag 26 to displace within the bag 26. Thus, the mix assisting assembly 2500 may also be a particulate identification assist assembly where bags 26 are checked for particulate via a machine vision system. A mix assisting assembly 2500 may be included in any location within a system 10, however, in certain examples, a mix assisting assembly 2500 may be included as a part of any fill station 2600 (see, e.g., FIG. 133) described herein. Alternatively, a mix assisting assembly 2500 may be a separate station which a bag 26 may be passed to after a fill station 2600. This may allow a bag 26 to be mixed while another bag 26 is being filled at a given fill station 2600.

Certain example mix assisting assemblies 2500 may displace the entire bag 26 in order to encourage generation of a homogenous solution within the bag 26. The bag 26 may for example be rotated about an axis by the mix assisting assembly 2500 to engender movement of the fluid within the bag 26. This movement may generate eddies which may persist after the bag 26 has been rendered stationary helping to thoroughly mix contents of the bag 26.

Referring now to FIGS. 152A-C, another exemplary mix assisting assembly 2500 is depicted. The example mix assisting assembly 2500 displaces the entire bag 26 to engender mixing of solution within the bag 26. As shown in FIGS. 152A-C, the bag 26 may be held in place on a rotary displacement stage 3312 included in the mix assisting assembly 2500. A bag retainer assembly 3310 may, for example, be coupled to the rotary displacement stage 3312 to capture the bag 26. Example bag retainer assemblies 3310 may have arms or clips which hold the bag 26 in place. A gripper or the like may be included to hold the ports 392 of the bag 26 in certain examples. A cradle 3311 or the like may also be included in the bag retention assembly 3310. The cradle 3311 may be coupled to the rotary displacement stage 3312 and may have a depression which is sized and shaped to accept at least a portion of a filled bag 26. The cradle 3311 and bag retention assembly 3310 may substantially prevent the bag 26 from displacing relative to the rotary displacement stage 3312. The gripper, clips, arms, etc. of the bag retention assembly 3310 may be mounted to the cradle 3311. One or more sensor 3314 which outputs a signal which varies in relation to the position of the rotary displacement stage 3312 may be monitored by a control system 15. The control system 15 may command displacement of the rotary displacement stage 3312 based upon the sensor 3314 output. In the example embodiment, the rotary displacement stage 3312 displaces the bag 26 such that the bag 26 rotates about an axis substantially perpendicular to a plane of the bag 26 in which the axes of the ports 392 fall. The rotation axis may be positioned so as to extend through a central location of the bag 26. The rotary displacement stage 3312 may be commanded to displace the bag 26 to one or more preset position. Typically, the bag 26 may be rotated to a sequence of predefined positions. The rotation may be alternated between clockwise and counterclockwise rotational directions between positions of the sequence of predefined positions. The bag 26 may be rotated at at least a certain rate as the bag 26 is rotated from one position to another. The bag 26 may remain stationary at each of the predefined positions for a period of time. In certain examples, the bag 26 may be rotated to a position at least 180° from its starting position (a fully inverted position) as the bag 26 is displaced with the mix assisting assembly 2500.

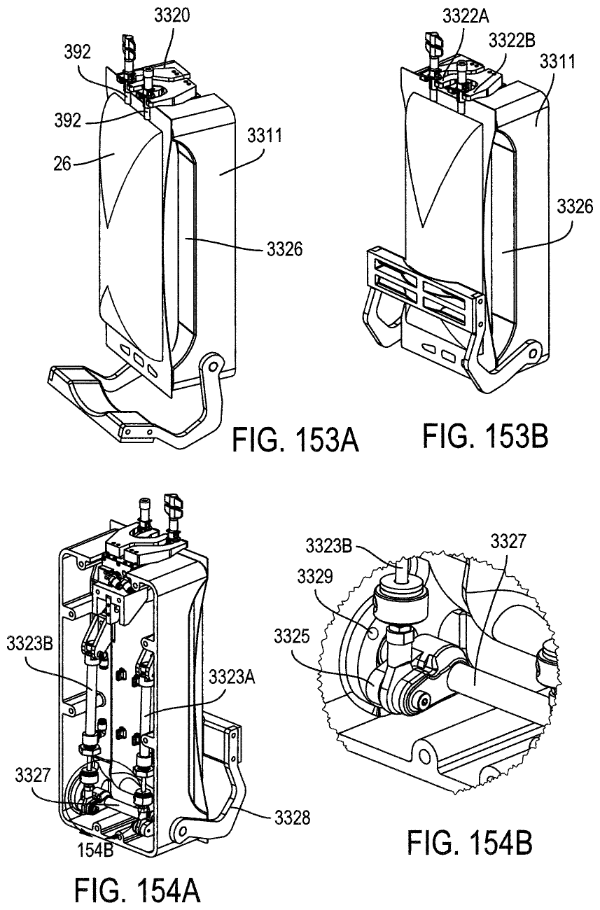

Referring now to FIGS. 153A-154B a number of views of an example bag retention assembly 3310 which may be included in a mix assisting assembly 2500 are depicted. As shown, the bag retention assembly 3310 includes a cradle 3311 which may include mounting points or hardware to couple the bag retention assembly 3310 to a rotary displacement stage 3312 (see, e.g., FIGS. 152A-B). The cradle 3311 may include a trough flanked by a set of opposing sidewalls 3326 which may support a bag 26 as the bag 26 is rotationally displaced. A gripper 3320 is attached to the cradle 3311 and may be actuated (e.g. pneumatically) to open and close a set of jaws 3322A, B for each port 392 included on the bag 26.

The bag retention assembly 3310 may also include a displaceable holder 3328 which may be displaced from a retracted position (see, e.g., FIG. 153A) and a deployed position (see, e.g., FIG. 153B). The holder 3328 may swing about an axis which runs substantially parallel to the width dimension of the bag 26. With the holder 3328 in the retracted position, the cradle 3311 may be accessible for a gantry or other robotic manipulator to install or remove a bag 26 from the mix assisting assembly 2500. Any imagers 3518 (see, e.g. FIG. 159) of a particulate inspection system 3500 (see, e.g. FIG. 159) may also have a clear field of view of the bag 26 when the holder 3328 is in the retracted position. In the deployed position, the holder 3328 may contact and press against the bag 26. This may firmly retain the bag 26 against the cradle 3311 to inhibit movement of the bag 26 relative to the cradle 3311 as the bag 26 is rotated. In some examples, the surface of the holder 3328 which contacts the bag 26 may be formed of or covered with a material with a high coefficient of friction against the bag 26 material. This may further assist in preventing undesired movement of the bag 26 as the bag 26 is rotated.

Referring now primarily to FIGS. 154A-B, the cradle 3311 may form a housing for one or more actuator 3323A, B (e.g. pneumatic cylinders) of the bag retention assembly 3310. A rear panel of the cradle 3311 is hidden in FIGS. 154A-B to depict the interior of the housing. The rotary displacement stage 3312 (see, e.g., FIGS. 152A-C) may couple to the rear panel when assembled. The actuators 3323A, B may be driven to displace respective linkages 3325 coupled to a pivot body 3327 to which the holder 3328 is fixedly coupled. As the actuators 3323A, B act on the linkages 3325 rotation of the pivot body 3327 and thus displacement of the holder 3328 is engendered. One or more position sensor 3329 may be included to monitor the position of the holder 3328. A rotary encoder may for example output a signal which varies in relation to the rotational position of the pivot body 3327 in certain examples. Alternatively, where the actuators 3323A, B are pneumatic cylinders, a cylinder proximity sensor (reed switch, Hall effect sensor, magnetoresistive sensor (AMR or GMR), or other magnetic sensor monitoring a magnet coupled to the cylinder) may be used.

Referring now to FIGS. 155A-156B, another example of a bag retention assembly 3310 which may be included in a mix assisting assembly 2500 is depicted. As shown, the bag retention assembly 3310 includes a cradle 3311 in which bags 26 may be partially seated when in place at a mix assisting assembly 2500. A gripper 3320 may be coupled to the cradle 3311. The gripper 3320 may have jaws 3322A, B which may be actuated (e.g. pneumatically) to open and close around ports 392 of a bag 26. Additionally, the bag retention assembly 3310 includes a holder in the form of a set of doors 3400A, B. Each door 3400A, B may swing about a pivot point which runs substantially parallel to the longitudinal axis of the bag 26. The doors 3400A, B may be displaceable from an open state (see, e.g., FIG. 155A) to a closed state (see, e.g., FIG. 156A). In the open state, a robotic manipulator or gantry may have a clear path to deposit or retrieve a bag 26 from the bag retention assembly 3310. In the closed state, the bag 26 may be firmly held against the cradle 3311.

In some examples, the doors 3400A, B may also be displaceable to a third, overly closed position. With a bag 26 present in the bag retention assembly 3310, the bag 26 may present an interference to displacement of the doors 3400A, B to this third position. The control system 15 may monitor the position of the doors 3400A, B to confirm a bag 26 is appropriately in place in the bag retention assembly 3310. One or more sensor may be included for this purpose (e.g. at least one sensor may monitor each door 340A, B). In the event that a bag 26 is expected to be present and a door position sensor indicates one or more door 3400A, B has reached the third position (or is within a predefined range of the third position), the control system 15 may generate an error notification. This may indicate that a bag 26 is improperly retained in the bag retention assembly 3310 or a bag 26 is absent when expected to be present.

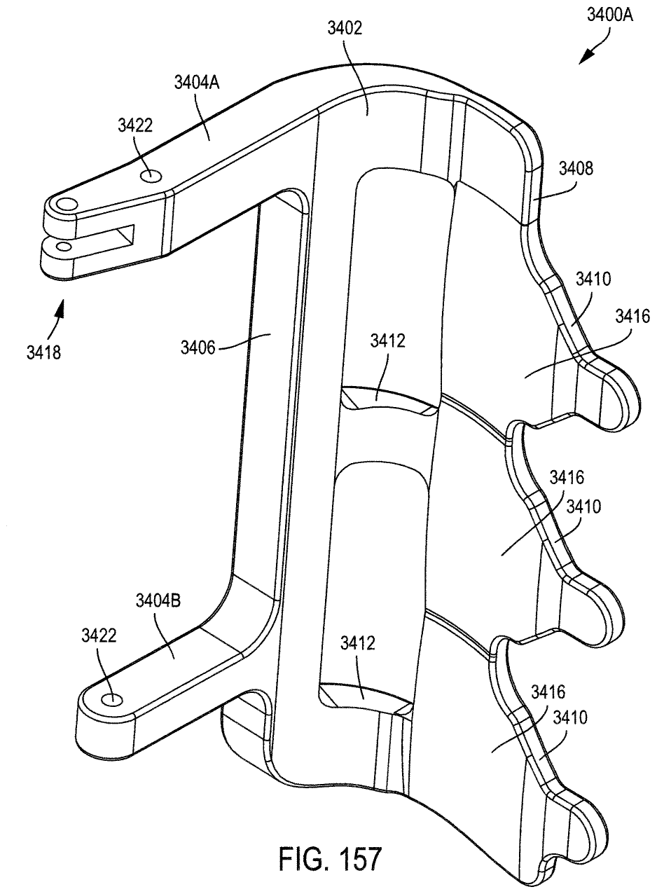

Referring now also to FIG. 157, a perspective view of a door 3400A in isolation is depicted. Each of the doors 3400A, B may be identical to one another for sake of simplicity, though this need not be the case in all examples. In examples where the doors 3400A, B are identical, the doors 3400A, B may be installed in a state where they have been flipped 180° from one another on opposing sides of the cradle 3311. The doors 3400A, B may be coupled to the cradle 3311 so as to swing in symmetric displacement paths lateral to the cradle 3311.

Still referring to FIGS. 155A-157, each door 3400A, B may include a main body 3402. The main body 3402 may, though need not, include one or more aperture 3412. A set of arms 3404A, B may project from opposing end regions of a first side 3406 of the main body 3402 of each door 3400A, B. One arm 3402A may be longer than the other and include an actuator coupling 3418 at its terminal end for coupling the arm 3402A to the output 3422 of a door actuator 3420A, B. Each arm 3402A, B may include a pivot pin hole 3422 via which the respective arm 3402A, B may be pivotally coupled to the cradle 3311. The pivot pin holes 3422 may be substantially coaxial.

A plurality of protrusions 3410 may extend from a second, opposing side 3408 of the main body 3402. The protrusions 3410 may be spaced apart by gaps which mimic the shape of the protrusions 3410. Thus, when the doors 3400A, B are closed (see, e.g., FIG. 156A), the protrusions 3410 of each door 3400A, B may be received in the gaps between the protrusions 3410 of the opposing door 3400A, B. This interlocking arrangement may assist in preventing pinching of the bag 26 when the doors 3400A, B are actuated. In the example embodiment, each door 3400A, B includes three protrusions 3410. All corners on the protrusions 3410 may be rounded. Each protrusion 3410 may include a base region. A rounded transition may be present between the base region and the end of the door 3400A, B from which the protrusion 3410 projects. The base region of the protrusions 3410 may taper thinner and include a rounded transition to a nub at their terminal ends. The tip of the nub of the protrusions 3410 may also be rounded. The doors 3400A, B may generally taper thinner in thickness as proximity to the side of the doors 3400A, B including the projections 3410 increases.

As shown, the main body 3402 may be arcuate in cross section such that the arms 3402A, B extend in a direction which is 80-100° from the direction of extension of the protrusions 3410. This arcuate shape may assist in providing a rolling contact against the exterior surface of the bag 26 as the doors 3400A, B are displaced to a closed position against the bag 26. A portion of the main body 3402 and protrusions 3410 may include a contact surface which abuts a bag 26 in the mix assisting assembly 2500 when the doors 3400A, B are in a closed state. As shown, one or more panel of gripping material 3416 which has a high friction coefficient with respect to the bag 26 may be placed on the contact surface. Additionally, the protrusions 3410 may at least partially extend in a plane which is offset from the second side of the main body 3402. When the doors 3400A, B are in a closed state, the portion of the doors 3400A, B formed by the protrusions 3410 may thus be more proximate the midplane of the bag 26 in which the ports 392 fall. Force exerted by the doors 3400A, B against the bag 26 may thus be concentrated along a centerline of the bag 26. This may depress the medial portion of the bag 26 pushing fluid within the bag 26 laterally.

As shown best in FIG. 155B and FIG. 156B, the output 3424 of each door actuator 3420A, B may be coupled to an actuator coupling 3418 on a respective door 3400A, B. In the example, the outputs 3424 are pivotally coupled such that the respective door 3400A, B may pivot relative to the output 3424 to which it is coupled. Each door actuator 3420A, B may also be pivotally coupled to the cradle 3311. Each door actuator 3420A, B may pivot relative to the cradle 3311 as the door 3400A, B is driven through its displacement range by the door actuator 3420A, B. In the example embodiment, the end of each door actuator 3420A, B opposite the output 3424 is coupled to a hinge 3430 extending laterally from the cradle 3311. A position sensor 3426 may be associated with each door actuator 3420A, B and may output a signal indicative of the position of the respective door 3400A, B as the door actuator 3420A, B is driven. The data signals from these sensors 3426 may be monitored by the control system 15 to determine whether the doors 3400A, B are in the open, closed, or in an overly closed position as mentioned above. In the example, the door actuators 3420A, B are pneumatic actuators and the position sensors 3426 may be magnetic sensors tracking the position of a magnetic body on the pneumatic cylinders. Any such sensor variety described herein may be used. Additionally shown in FIG. 155B and FIG. 156B is a mount point 3428 for coupling of the cradle 3311 to a rotary displacement stage 3312 or assembly. The mount point 3428 is shown coupled to a rotary actuator 3470 in FIG. 160C for example.

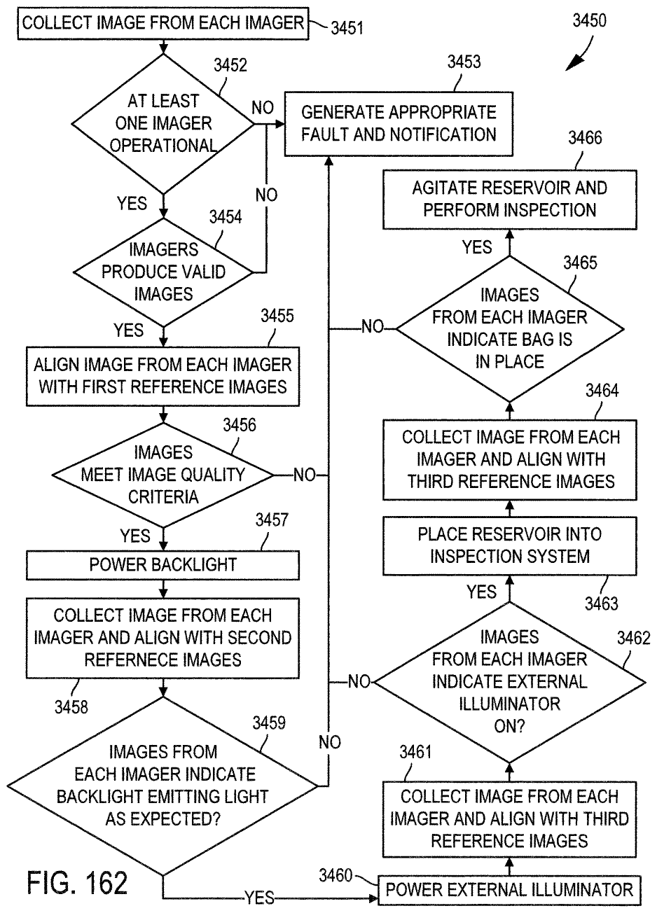

Referring now to FIG. 158, a flowchart 3380 is depicted detailing a number of example actions which may be executed to displace a bag 26 to encourage creation of a homogenous solution within the bag 26. As shown, in block 3381 a bag 26 may be displaced to the mix assisting assembly 2500. This may be accomplished by a robotic gripper or gantry under direction of the control system 15. Where the system 10 performs a functionality check of the mix assisting assembly 2500 and/or particulate inspection system 3500 (see, e.g., FIG. 162), this functionality check may be required to have been successfully completed before a bag 26 is displaced to the mix assisting assembly 2500 in block 3381.

In block 3382, the bag 26 may be retained in the mix assisting assembly 2500 by a bag retention assembly 3310. This may include actuating jaws 3322A, B of the gripper 3320 attached to the cradle 3311 to close upon and retain the ports 392 of the bag 26. Additionally, this may include actuating doors 3400A, B or a displaceable holder 3328 against the bag 26. The control system 15 may verify the output signal from at least one bag retention assembly sensor indicates that a bag 26 has been successfully retained in bag retention assembly 3310. Where the gripper 3320 is paired with at least one bag sensor 4312A, B (see, e.g., FIG. 68B), the control system 15 may confirm the output signal(s) of the at least one bag sensor 4312A, B is/are indicative of ports 392 being held in the jaws 3322A, B. The output from any jaw position sensor(s) 4309 (see, e.g., FIG. 71) may also be checked by the control system 15 to confirm the jaw 4308A, B positions are consistent with a bag 26 being held. The control system 15 may further check to confirm the output signal of at least one position sensor 3426 monitoring the bag doors 3400A, B or displaceable holder 3328 is indicative of a bag 26 being present within the bag retention assembly 3310 (further described, e.g., in relation to FIGS. 155A-156B). The output of at least one bag sensor 4312A, B of the gantry gripper 4204 may also be check to verify absence of a bag 26 in the gantry gripper 4204. In the event that the control system 15 determines a bag 26 is not retained or correctly held in the mix assisting assembly 2500, the control system 15 may generate a notification. The system 10 may be transitioned to a standby state until an indication that the issue is resolved has been received by the control system 15.

In the example shown in FIG. 158, the bag 26 may be rotated between pairs of preset clockwise and counterclockwise positions. The order in which the bag 26 is displaced to the clockwise position and counterclockwise position in each pair may differ from embodiment to embodiment. The bag 26 may be displaced in a first rotational direction to the first position of a pair and rotated in a second, opposing rotational direction to the second position in that pair. FIG. 158 describes displacing the bag 26 to the clockwise position and then to the counterclockwise position though this merely exemplary.

The bag 26 may be rotated between each position in a pair some preset number of times. For some pairs, the number of iterations may be only a single iteration. For other pairs, the bag 26 may be rotated between each position in a pair a plurality of times (e.g. three times). A control system 15 may govern operation of a rotary actuator 3470 (see, e.g. FIG. 159) based on data from at least one rotational position sensor 3472 (see, e.g., FIG. 159) to rotate the bag 26 to any preset positions. The rotation rate of the bag 26 may be controlled to a predefined rate. In certain examples, the bag 26 may be rotated at a rate of 200-300 degrees per second (e.g. 250 degrees per second) during transitions between all positions. In certain examples, the bag 26 may be displaced between preset positions at rates specific to each individual transition between positions. The specified rates may differ depending on the transition, though at least some of the individual transitions may be specified to occur at the same rate. There may be a dwell period where the bag 26 is kept substantially stationary. This dwell period may elapse after the bag 26 is rotated to each preset position. In the example, a predefined dwell period is allowed to elapse each time the bag 26 is displaced to a new position (see, e.g., blocks 3383, 3384, 3387, 3388). The dwell time period may be specified for each individual displacement of the bag 26 or a dwell time for use with each pair of positions may be specified in non-limiting examples. Target accelerations and decelerations may also be specified for each individual displacement of for each set of positions pairs. A set of position pairs, rotation/acceleration/deceleration rates, and dwell times may be referred to as a motion profile.

Conformity to the motion profile may be tracked for each bag 26 and written to the batch record for each bag 26 by the control system 15. Deviation data may be collected as the bag 26 is displaced through the motion profile. For example, the deviation from the target positions for each displacement, deviations from the target velocity for each displacement, and the deviations from the target acceleration/deceleration for each displacement may be captured and documented in the batch record. Alternatively, average deviations for each position pair may be used. The information may be collected from a rotational position sensor 3472 as a bag 26 is displaced through a motion profile. The deviation for the target dwell time may also be tracked for each displacement (or the average for each position pair). If motion of the bag 26 does not meet predefined conformity criteria, mixing of the bag 26 may be determined to have failed. In the event that the any of the deviations exceed respective predetermined thresholds, mixing of the bag 26 may be determined to have failed. A failure (and supporting deviation data) may be documented in the batch record for the bag 26 by the control system 15. The control system 15 may place the system 10 in a standby state and generate a notification that the mix assisting assembly 2500 requires service in the event of a failure.

In some examples, the type of bag 26 in place at the mix assisting assembly 2500 may be known. For example, the bag 26 may have an identifier (see, e.g., indicium 56 of FIG. 159) which is read by an imager, barcode reader, etc. in communication with the control system 15. Bag 26 identifying information may also be manually input via a user interface of the system 10 in certain examples. The motion profile used for a given bag 26 may be chosen by the control system 15 based on the type of bag 26 in place at the mix assisting assembly 2500. A look-up table or the like may be used to facilitate selection of a motion profile based on a bag 26 identity. For example, the position pairs may be held constant across bags 26. Maintaining the same dwell times for each rotation pair, the rotational rate may be increased as the volume of the bag 26 increases. Alternatively, dwell times could be increased while maintaining the rotational rate constant for each position pair as bag 26 volumes increase. It is not necessary that only one of the dwell time and rotation rate values be adjusted; certain implementations may adjust both values as bag 26 volume increases.

Without being tied to any particular theory, it is believed that rotating the bag 26 to a given position generates primary or initial eddies within the fluid contained in the bag 26. The size of the primary eddies may generally be a factor of the amount and rate of angular displacement for a given reservoir (e.g. bag 26). As the bag 26 dwells in position, the primary eddies may decay into smaller eddies due to inertial instabilities. These eddies have a smaller Reynolds number and thus are less responsive to changes in momentum of the bag 26. These eddies may primarily respond to the viscosity of the surrounding solution rather than rotation of the bag 26. When the bag 26 is rotated to the opposing position of a position pair, the intervening dwell period may allow more of the energy from the counter-rotation to go towards creating new primary eddies instead of devoting some of the energy to slowing down existing primary eddies. Additionally, the remaining smaller eddies from previous preset positions may feed into the advective mixing action engendered by the primary eddies induced as the bag 26 is rotated to subsequent preset positions. As the bag 26 continues to be rotated between positions, the eddies formed within the bag 26 may mix fluid within the bag 26 with greater and greater vigor. The primary eddies formed may act on the fluid in conjunction with the mixing effects provided by secondary, tertiary, quaternary, etc. eddies from previous rotations. Additionally, as the bag 26 is rotated a greater magnitude from the starting position, the primary eddies formed may become larger resulting in larger child eddies (and longer cascades of progeny child eddies) as they break down. Thus repeated displacement of the bag 26 between positions of increasing (though not necessarily strictly increasing) magnitude may efficiently and quickly homogenize fluid within a bag 26 and dissolve any solid constituent within the bag 26.

Still referring to FIG. 158, the bag 26 may be rotated clockwise from a starting position to a first preset position in block 3383. The bag 26 may be rotated counterclockwise to a second preset position in block 3384. In some examples, the first preset position may be 40° from the starting position and the second preset position may be −40° from the starting position. The magnitude of rotational displacement from the starting position for the clockwise and counterclockwise position for each position pair may not be equal in all embodiments. In certain examples, the bag 26 may dwell at each of the first and second preset positions for less than half a second (e.g. 0.275-0.325 seconds or 0.390-0.410 seconds) for a one liter bag 26. The control system 15 may determine any dwell times (and/or rotation rates) based on bag 26 volume in certain embodiments (e.g. via a look up table).

If, in block 3385, the preset number of displacement iterations between the first and second position has not been met, the bag 26 may again be rotated between the first and second position in blocks 3383, 3384 and the accompanying dwell periods may be allowed to elapse. In some examples, the bag 26 may be displaced three times between the first and second positions. In certain implementations, the number of iterations may depend on the type of concentrate included in the bag 26. A greater number of iterations may be used for bags 26 which were filled with solid constituent. Additionally, a greater number of iterations may be used in the event that the bag 26 was filled with liquid concentrate and a particulate inspection system 3500 (see, e.g., FIG. 159) determined the presence of solid precipitate.

After the preset number of displacement iterations has been satisfied in block 3385, the bag 26 may be rotated clockwise and counterclockwise to the next preset positions in blocks 3387, 3388 (if in block 3386 there are additional preset positions). Dwell periods associated with the positions for blocks 3387, 3388 may be allowed to elapse. The bag 26 may be rotated between this next pair of positions until a preset number of displacement iterations has been achieved in block 3389. This may repeat until the bag 26 has been displaced to all predefined positions.

Typically, the magnitude of rotational displacement may increase with each subsequent pair of preset positions. The dwell time may also increase as the magnitude of displacement increases. In certain embodiments, the bag 26 may be rotated to a third and fourth position. The third position may be 85° from the starting position and the fourth position may be −85° from the starting position. The bag 26 may dwell at each of the third and fourth position for less than 0.75 seconds (e.g. 0.6-0.7 seconds). The bag 26 may also be rotated to fifth and sixth positions. The fifth position may be 120° from the starting position and the sixth position may be −120° from the starting position. The bag 26 may dwell at each of the fifth and sixth position for less than a second (e.g. 0.775-0.825 seconds). The bag 26 may further be rotated between seventh and eight positions. The seventh position may be 180° from the starting position and the eighth position may be −180° from the starting position. The dwell time at each of the seventh and eighth positions may be less than seven seconds (e.g. 4-6 seconds). In other examples, a lesser or greater number of positions pairs may be used.

The number of displacement iterations between each pair of positions may decrease (though non-strictly in certain examples) as the magnitude of rotational displacement increases. The number of displacement iterations between the last pair of positions may be lower than the number of displacement iterations for the first pair of positions. At least for bags 26 where the constituent within the bag 26 is a crystalline solid or powder, the number of displacement iterations may be greater for pairs of positions where the bag 26 is rotated less than 90° from its starting position. This may prevent constituent from displacing and lodging into a port 392 of the bag 26.

Still referring to FIG. 158, once, in block 3386, the bag 26 has been displaced through all preset positions, the bag 26 may be returned to the starting position in block 3390. In some embodiments, the control system 15 may track a number of bags 26 mixed by the mix assisting assembly 2500. In such examples, the control system 15 may increment also increment the count of bags 26 mixed in block 3390. Depending on the embodiment, the bag 26 may be monitored for particulate in block 3391. A wait period may optionally be allowed to elapse prior to any such monitoring. Additionally or alternatively, a maximum wait period may be enforced. The control system 15 may ensure that the period of time since completion of the motion profile is not greater than a preset time prior to orchestrating any such monitoring. In block 3391, a control system 15 may, for example, analyze one or more image of the bag 26 supplied by one or more imager 3518 (see, e.g., FIG. 159) to determine whether particulate is present. Particulate monitoring will be further described later in the specification (see, e.g., FIG. 159). The bag 26 may also be checked to determine whether any solid constituent present in the bag 26 has dissolved in block 3391. In certain embodiments, the bag 26 may be rotationally displaced to further positions in the event that undissolved constituent is determined to be present within the bag 26. This may repeat until the control system 15 determines all solid constituent in the bag 26 has been dissolved in block 3391. In some examples, it may be determined that the particulate is insoluble in the event that particulate is continually detected after further displacement of the bag 26. After a certain number of additional displacements or a supplement motion profile has been completed, it may be determined any remaining particulate is insoluble. Detection of insoluble particulate may result in the control system 15 flagging the bag 26 for rejection.

In alternative embodiments, the bag 26 may be rotated between a set of positions or sets of positions prior to block 3383 and the bag 26 may be subsequently monitored for particulate. This may create movement of particulate within the bag 26 which may assist in detection of the particulate with a particulate detection system 3500 (see, e.g., FIG. 159). The number of rotation iterations for each position pair may be adjusted by the control system 15 based upon the particulate analysis. The control system 15 may also halt operation of the mix assisting assembly 2500 in the event that certain undesired particulate is determined to be present. For example, in the event that insoluble foreign particulate is detected, the control system 15 may flag the bag 26 as rejected and the bag 26 may be directed to quarantine without mixing.

After mixing and inspection is complete, the control system 15 may command actuation of the gantry assembly 4200 to collect the bag 26 from the bag retention assembly 3310. The control system 15 may check the output from at least one bag sensor 4312A, B of the gantry gripper 4204 to verify the gantry gripper 4204 is closed upon ports 392 of a bag 26. The doors 3400A, B of displaceable holder 3328 may be actuated out of contact with the bag 26 in the bag retention assembly 3310. The output signal of the at least one position sensor 3426 may be checked by the control system 15 before attempting to remove a bag 26 from the bag retention assembly 3310 with the gantry grasper 4204. It may be verified the doors 3400A, B or displaceable holder 3328 are in a withdrawn state away from the bag 26 before the gantry assembly 4200 is actuated to remove the bag 26. Where the gripper 3320 is paired with at least one bag sensor 4312A, B (see, e.g., FIG. 68B), the control system 15 may confirm the output signal(s) of the at least one bag sensor 4312A, B is/are indicative of ports 392 being absent in the jaws 3322A, B after the gantry gripper 4204 has been displaced away from the bag retention assembly 3310. The bag 26 may be marked and deposited in an outfeed receptacle 4120A, B (see, e.g. FIG. 221) by the gantry assembly 4200 based on data in the batch record built for that bag 26.

Referring now to FIG. 159, a diagram of an example particulate inspection system 3500 is depicted. Though example particulate inspection systems 3500 are described in relation to embodiments of systems 10 described herein, example particulate inspection systems 3500 may be implemented as stand-alone systems or be integrated into other systems. Additionally, the example particulate inspection systems 3500 are described in relation to flexible reservoirs (e.g. IV bags) typical of the variety used in in medical settings. These reservoirs are generally described in the context of injectable fluids. This is merely exemplary. The example particulate inspection systems 3500 described herein may be utilized with other varieties of reservoirs used in a wide variety of applications. For example, rigid reservoirs such as vials, jars, bottles, etc. may be used. The example particulate inspection systems 3500 described herein may also be used with reservoirs which include both rigid and flexible portions. Reservoir retention hardware of the particulate inspection systems 3500 may be adapted to retain the desired reservoir variety. Additionally, the displacement profile during mixing may be adjusted based on the subject reservoir. Applications may include preparation of medical solutions, laboratory solutions, and biological applications such as cell culturing, biomanufacturing, etc. Applications may also include particulate detection for industrial products (e.g. oils conforming to a stringent ISO cleanliness code). Examples described herein are generally described in relation to liquids, though may be used to detect particulate in other fluids such as gases. Though referred to herein as particulate inspection systems 3500, it will be clear that such systems are also capable of detecting, for instance, bubbles and may be utilized as bubble detection systems for reservoirs. Particulate inspection systems 3500 may also be referred to herein as reservoir content detecting and/or differentiating systems. Particulate inspection systems 3500 described herein typically detect, track, and classify contents of interest within reservoirs. In certain alternatives, a particulate inspection system 3500 may perform a subset of these activities. Some particulate inspection systems 3500 may detect and optionally classify components of interest without necessarily tracking them.

In general, particulate inspection (or content detection and differentiating in general) of reservoirs is a particularly difficult challenge. Commonly it is accomplished with a human inspector which engenders a high degree of subjectivity. Best practices require frequent breaks, recurring training, and other recurring reverification (e.g. eye exams) for inspectors. Even so, FDA data indicates that around one third of recalls for sterile injectables are related to the presence of particulate. Within the time period of 2010-2021 there were between 6 and 25 recall events per year relating to visible particulate in sterile injectables. A relatively small footprint automated particulate inspection system which is not prohibitively expensive would be welcome.

Particles or other content of interest for identification may be quite small. For example, it may be desired that a particulate inspection system 3500 identify particles of 200 μm or less (e.g. 100 μm or less or even 50 μm or less) at a level which at least achieves parity with a human inspector. These particles may exist in a solution which also contains gas bubbles. Some microbubbles within the solution may be in the same size range as the particulate of interest making differentiation between particulate and bubbles a challenge. False rejection of a reservoir due to misidentification of an air bubble as particulate is undesirable. It may be particularly undesired in scenarios where storage for rejected reservoirs is limited and is access controlled or in an environmentally controlled enclosure. Excessive false rejection may require a user intervention with a fluid packaging system and may limit throughput of a parent system 10.

Adding to the challenge, certain particulate may possess properties which are not ideal for visual detection. Some particulate (e.g. particulate from IV bag material) may be substantially transparent or have a high degree of translucence. Other particulate may absorb or reflect substantial amounts of light making it difficult to view against certain backgrounds. The density of particulates may also present challenge as certain particulate may tend to collect at the top or bottom of a reservoir. Head space in a reservoir may further complicate detection. The type of reservoir can also present optical challenges. IV bags, for example, are flexible and malleable. Each bag will tend to crease and seat differently when installed in a particulate inspection system 3500 giving respective bags differing optical properties. Reservoirs may also include print (e.g. text, logos, etc.) which may obfuscate view into the reservoir. Some reservoirs such as IV bags may also display a tendency to settle or shift over time while observations are made.

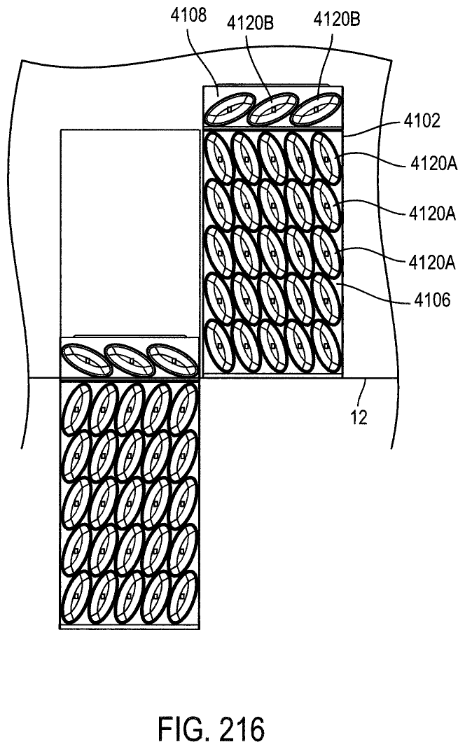

Exemplary particulate inspection systems 3500 described herein may image a filled bag 26 and perform machine vision analyses to identify contents of interest within the fluid contained in a bag 26. Particulate inspection systems 3500 may also differentiate between different types of contents of interest within the fluid. Bubbles of air or gas may be differentiated from solid particulate for example. In certain embodiments, particulate inspection systems 3500 may also differentiate between different types of particulate. A particle of undissolved constituent (e.g. salt, sugar, etc.) may be differentiated from other types of particulate for instance. Any determination as to the presence of at least certain contents of interest may be documented in a log which may be committed to a database 3502 or other memory. A determination as to whether the subject bag 26 meets one or more acceptance criteria may also be made. This determination, any raw data, analyzes, digests thereof, summaries, etc. may be included in the log communicated to the database 3502. In the event that the bag 26 fails acceptance criteria, the bag 26 may be routed to a discard or quarantine destination in the system 10. This could for example be dedicated, access controlled receptacles 4120B or an outfeed drawer 4102 in a system 10 (see, e.g., FIG. 216). The bag 26 may fail acceptance criteria in the event that analysis is indicative that undesired contents of interest such as particulate are determined to be present in the bag 26.

As shown, example particulate inspection systems 3500 may be disposed within an enclosure 12. The enclosure 12 may be substantially light tight. Thus, ambient light may be obstructed from reaching the interior of the particulate inspection system 3500. The interior of the enclosure 12 may be unlit, but for light generated by components of the particulate inspection system 3500. In some embodiments, the enclosure 12 may house one or more additional station of a system 10 (e.g. a marking assembly 3700, outfeed assembly 4100, etc.). In such embodiments, light generating components in these stations may be minimized. There may for example be one or more small, low lumen output LED indicator light on components in the enclosure 12 while still considering the enclosure 12 to be unlit. To the extent other stations which utilize lighting are present within the enclosure 12, the particulate inspection system 3500 may not image bags 26 coincident with lighting in other stations being powered. Shielding or masking of light emitting components within the enclosure 12 may also be implemented. The particulate inspection system 3500 may be within a partitioned portion of the enclosure 12 separate from light producing components. An irradiation assembly 2608 with antimicrobial light emitters 2610 may be in a processing compartment 4300 while the particulate inspection system 3500 is in a separate outfeed compartment 4202 for example.

The enclosure 12 may be environmentally controlled to limit microbes, detritus, and humidity in certain examples. Where the particulate inspection system 3500 receives bags 26 from a fill station 2600, the enclosure 12 in which the particulate inspection system 3500 is disposed may be environmentally controlled to a less stringent level than the enclosure 12 in which the fill station 2600 (see, e.g., FIG. 105) is disposed. Alternatively, the enclosure 12 may not be subjected to environmental control. A transfer chamber 3506 (see, e.g., FIG. 2) may be disposed intermediate a processing compartment 4300 of the enclosure 12 including the fill station 2600 and compartment of the enclosure 12 including the particulate inspection system 3500 (see, e.g., outfeed compartment 4202 of FIG. 2). The transfer chamber 3506 may include a set of doors 4700, 4702 which may be opened via respective actuators under control of the control system 15. The control system 15 may inhibit both of the doors 4700, 4702 from being in an open state at the same time.

The particulate inspection systems 3500 may include a retainer assembly for holding a bag 26. In the example embodiments described herein, the retainer assembly is described as a mix assisting assembly 2500 (further described in relation to FIGS. 155A-158). Where a mix assisting assembly 2500 is described as part of a particulate inspection system 3500, it shall be understood that this is merely exemplary. The retainer assembly of a particulate inspection system 3500 may be a dedicated assembly and a system 10 may include a separate mix assisting assembly 2500. Features of mix assisting assemblies 2500 described in relation to example particulate inspection systems 3500 disclosed herein may be included in a dedicated retainer assembly for a particulate inspection system 3500. Additionally, any of the features of the mix assisting assemblies 2500 described in relation to the particulate inspection system 3500 may be included in any other mix assisting assemblies 2500 shown or described herein.

As shown, the mix assisting assembly 2500 may include a cradle 3311 against which the bag 26 may be placed. A gripper 3320 may be attached to the cradle 3311 and may be actuated (e.g. pneumatically) to open and close a set of jaws around respective ports 392 of the bag 26. The cradle 3311 may be attached to a rotary actuator 3470 via a mount point 3428 (see, e.g., FIG. 155B). A motor encoder or other rotational position sensor 3470 may be included and may output a signal indicative of the rotational position of the cradle 3311. A holder may be attached to the cradle 3311. Any holder described herein may be used, though in the example embodiment, a set of doors 3400A, B (only one shown in FIG. 159, see, e.g., FIG. 155A) are included. The holder may be actuated to firmly retain the bag 26 against the cradle 3311 when the rotary actuator 3470 is driven.

The cradle 3311 may include a rest body 3510 which a surface of the bag 26 may contact when retained at the cradle 3311. The rest body 3510 may be contoured to mimic the shape of a filled bag 26. The rest body 3510 may be transparent or at least partially translucent. For example, rest bodies 3510 may be constructed of a transparent or at least partially translucent material. Alternatively, the rest body 3510 may be constructed of a transparent material to which a surface treatment is applied. The rest body 3510 may, for instance, be frosted. The cradle 3311 may also include an illuminator 3512. The illuminator 3512 may include at least one light source and may provide substantially uniform illumination of the rest body 3510. A diffuser may also be included. In certain examples, a polarizer 3503 may additionally or instead be included. When the illuminator 3512 is powered, a bag 26 in place on the cradle 3311 may be backlit. As shown in FIG. 159, the illuminator 3512 is positioned opposite the rest body 3510. Alternatively or additionally, the rest body 3510 may be side lit and/or otherwise lit from another vantage point. The illuminator 3512 may be a side emitting panel type light source with a built in diffuser. The illuminator 3512 may emit white light, though in some embodiments, the illuminator 3512 may emit specified wavelengths or may be adjusted to emit a selection of different color light(s) under the direction of a processor of the control system 15. The illumination output intensity of the illuminator 3512 may be adjustable.

The rest panel 3510 may be patterned. The pattern may include a collection of dark 3514A and light regions 3515B (see, e.g., FIG. 160A). The dark regions 3514A may be formed by masking or otherwise obstructing the passage of light through the rest body 3510. Thus, in various examples, the rest panel 3510 may include a collection of light obscuring regions and regions which permit passage of light. The dark regions 3514A may be black when the illuminator 3512 is powered. The light regions 3514B may be left uncovered or be unaltered regions of the rest body 3510. The light regions 3514B may be white (or substantially the color output by the illuminator 3512) when the illuminator 3512 is powered. The pattern may consist of alternating dark and light regions 3514A, B. There may be an even or approximately even distribution of dark and light regions 3514A, B within the pattern. In certain examples, a checker or chessboard pattern may be used. Other example patterns may include spirals, horizontal lines, vertical lines, polka dot, herringbone, houndstooth, a tessellation, zebra-pattern stripes, etc. In some preferred embodiments, a checkered pattern with 6-8 mm squares (e.g. 7 mm) may be utilized. The size of the dark and light regions 3514A, B may be selected based on expected typical content velocities and a desired frame capture rate for imagers 3518 of the particulate inspection system 3500. The illuminator 3512 may light the rest panel 3510 at a value of at least 3000 lux.

In alternative embodiments, the illuminator 3512 may be otherwise controlled to generate contrast. For example, the illuminator 3512 may be powered to generate contrast in backlight color over time. In one example, no pattern may be included on the rest panel 3510, and the illuminator 3512 may be rapidly switched on and off, transitioned through different colors, or transitioned through different light output intensities at a high rate. This would similarly generate a contrasting light and dark background albeit with a temporal offset. The speed at which different illuminator 3512 set points are transitioned through may be dependent on the frame rate 3518 of any imagers 3518. The frame rate of the imagers 3518 may similarly be synchronized with the illumination transitions of the illuminator 3512. A transition between illuminator 3512 set points may occur a plurality of times per second. In some examples, ten or more transitions per second may occur.

In still other examples, a pattern (e.g. a chessboard pattern or any other pattern mentioned herein) may be produced on a display. In such examples, the pattern may be dynamic and may change during imaging by the imager 3518. In certain examples, the display may switch from a first pattern to a second pattern (and potentially other patterns). Though described as patterns, it would also be possible to use one or more solid background of a specific color (e.g. white, black, some preset color space value) in the series of transitions. The color(s) of a solid background (or portion(s) of a patterned background) may change at different time points. The same pattern may also be adjusted such that it is reduced/enlarged. For example, squares of a chessboard may be increased or decreased in size. A first pattern may be used for a first series or set of images and a second pattern may be used for a second series or set of images (and so on if desired). The first and second sets of images may be sets of consecutive images, but need not be in all examples. Any changes may be synchronized with the frame rate to ensure that the desired pattern is displayed for frames which will belong to a given set. Each pattern may be tuned for a particular content type (e.g., for certain varieties of particulate or for different content size thresholds).

An external illuminator 3516 may also be included in certain examples. The external illuminator 3516 may be separate from the cradle 3311 may be positioned to direct light into the bag 26. In the example embodiment, the external illuminator 3516 is an underlight. The external illuminator 3516 may emit collimated light or have light sources which emit light in a narrow beam angle. In certain examples, the external illuminator 3516 may include a number of light emitters (e.g. LEDs) and one or more lens. The lens may include collimating or Fresnel lenses for each of the light emitters such that the external illuminator light 3516 sources emit collimated light. It should be understood that the term collimated would encompass light output from such an arrangement and should not be interpreted as meaning strictly perfectly collimated. Narrow beam angle lenses may also be used. Where narrow beam angle is used herein, it refers to beam angles of 30° of less (e.g. no greater than) 20°. In other examples, the external illuminator 3516 may include light sources with wider beam angles. Additionally, in certain examples, at least one polarizer 3517 may be included. A polarizer 3517 may be included for the external illuminator 3516 and for each of the imagers 3518. Polarized light reduces glare off of certain reservoirs such as bags 26. Polarized light may also tend to better highlight air bubbles within a reservoir. Polarizers 3517 may not be included in all embodiments. Though shown as an underlight in the example, external illuminators 3516 may instead or additionally be positioned at other vantage points. For example, sidelight type external illuminators 3516 may be included in some examples.

The external illuminator 3516 may produce light of a specific color, wavelength, or band of wavelengths. For example, the external illuminator 3516 may produce red or orange light. In some embodiments, the external illuminator 3516 may output light in the 600-630 nm range. The color of light selected for the external illuminator 3516 may be chosen based on potential anticipated sources of particulate. Preferably, the color may be selected to differ from anticipated sources of potential particulate. The intensity of the light emitted by the external illuminator 3516 may be greater than the intensity of light emitted by the illuminator 3512 in the cradle 3311. In alternative examples, the color of light emitted by the external illuminator 3516 may be adjustable and may be user specified. Different colors may be emitted by the external illuminator 3516 at different time points during inspection of a bag 26 or other reservoir. The external illuminator 3516 may produce an illuminance of at least 20,000 lux (e.g. 23,000-25,000 lux). Each light emitter assembly 3522 (see, e.g., FIG. 160C) of the external illuminator 3516 may also emit at a narrow beam angle such as 5-15° (e.g. 10°).

Still referring to FIG. 159, exemplary particulate inspection systems 3500 may include at least one imager 3518. An imager array 3519 including three imagers 3518 is shown in FIG. 159. Each of the at least one imager 3518 may be positioned in opposition to the rest body 3510 such that a bag 26 is disposed intermediate the rest body 3510 and imager(s) 3518 when in place at the cradle 3311. Thus the pattern may be provided on a first side of the reservoir (e.g. bag 26) while the imager(s) 3518 may be positioned on a second, opposing side of the reservoir. Each of the at least one imager 3518 may include a CCD or CMOS sensor and may capture monochrome or color image data. Each imager 3518 may have a depth of field which is at least equal to the thickness of a filled bag 26. The f-number for the imager(s) 3518 may be kept under f-8 and preferably lower. This may limit the amount of diffractive blur introduced to any images generated by the imager(s) 3518. Each imager 3518 may preferably have an at least 10 megapixel camera (e.g. 12 megapixel). Each pixel may correspond to a square region at the midplane of a nominal bag 26 which includes the ports 392. For exemplary 1 liter bags 26, a 100 μm particle may fill a pixel of an image generated by an example imager 3518. Higher resolution image sensors (e.g. 64 MP) may be utilized in alternative examples.

In the example embodiment shown in FIG. 159, three imagers 3518 are included. The imagers 3518 are positioned at an angle other than perpendicular to the midplane of the bag 26 including the ports 392. In the example, the imagers 3518 are disposed such that the optical axis or viewing axis of each imager is inclined 30-35° (e.g. 32°) from an orientation perpendicular to the midplane of the bag 26 including the ports 392 or height dimension of the cradle 3311. A hypothetical spherical lens within the bag 26 receiving collimated light from an underlight type external illuminator 3516 may produce a light return which is particularly strong at this angle.

The imagers 3518 may each have a field of view which includes a portion of the bag 26. A first of the imagers 3518 may have a field of view including a portion of the bag 26 most proximal to the ports 392. Another of the imagers 3518 may have a field of view encompassing a portion of the bag 26 most distal to the ports 392. The remaining imager 3518 may have a field of view which captures the central portion of the bag 26 intermediate the fields of view of the former imagers 3518. Typically, the field of view of each imager 3518 may overlap with that of at least one other imager 2518. In some embodiments, the field of view of the imager 3518 dedicated to the central portion of the bag 26 may overlap with the respective fields of view of the adjacent imagers by at least 5% (e.g. 5-10%). Including multiple imagers 3518 may allow the particulate inspection system 3500 to remain relatively compact, however, fewer (e.g. a single imager 3518) may be used by spacing the imager 3518 a greater distance from the bag 26. In such examples, the imager sensor may have a greater pixel density (e.g. 20-30 megapixel or higher). Other or additional variables such as lens focal length may be adjusted to alter the number of imagers 3518 without necessarily changing the footprint of the particulate inspection system 3500. The focal length of one or more imager 3518 included in a particulate inspection system 3500 may be adjustable in certain embodiments. Such imagers 3518 may sweep through their focal length range as image data of the bag 26 is captured to collect in focus data at across various depths within the bag 26. The bag 26 may be placed in the particulate inspection system 3500 with any text or graphics of the bag 26 adjacent the rest body 3510 so that they are not intermediate the imagers 3518 and the interior of the bag 26. Any embodiments described or shown herein as having a certain number of imagers 3518 may be modified to include a greater of fewer number of imagers 3518.

In certain examples, each of the imager(s) 3518 may be associated with at least one filter 3542. For example, imagers 3518 may each be paired with a neutral density filter in some examples. Alternatively or additionally, a polarizer 3517 may be placed in front of the lens for each imager 3518. Where polarizers 3517 are used, the polarizers 3517 for each imager 3518 may be oriented at a lensed light transmitting orientation. This orientation is an angle related to the polarization angle of the light emitted from the external illuminator 3516. This orientation may differ for each imager 3518 and may substantially block light from the external illuminator 3516 reflecting off bag material and particulate within the bag 26. The lensed light transmitting orientation may be selected to permit transmission polarized light emitted from the external illuminator 3516 and lensed or reflected by a hypothetical spherical lens within the bag 26. In alternative embodiments, light from the illuminator 3512 may be polarized and the polarizers 3517 on each image may be oriented to at a cross-polarizing orientation. This may assist in better highlighting certain varieties of particles.

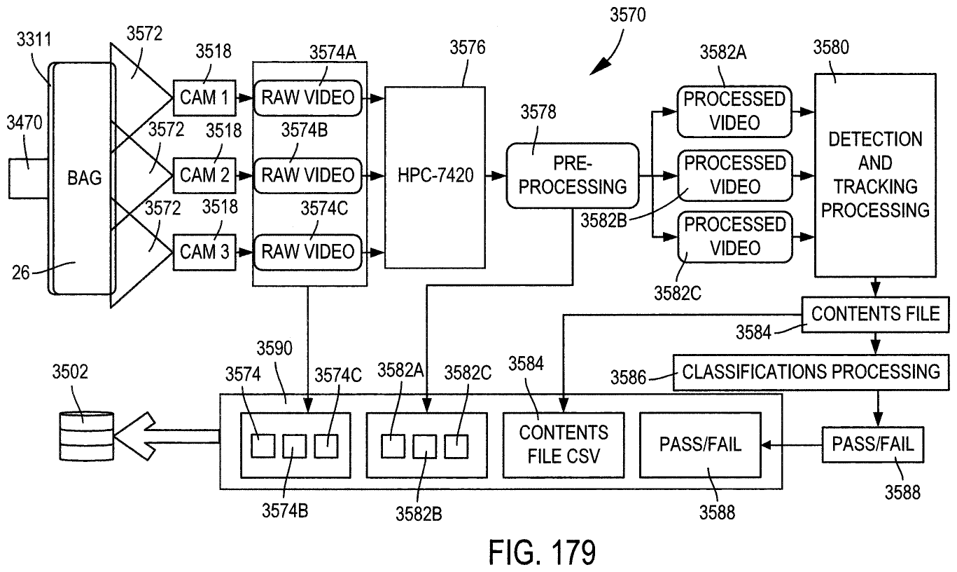

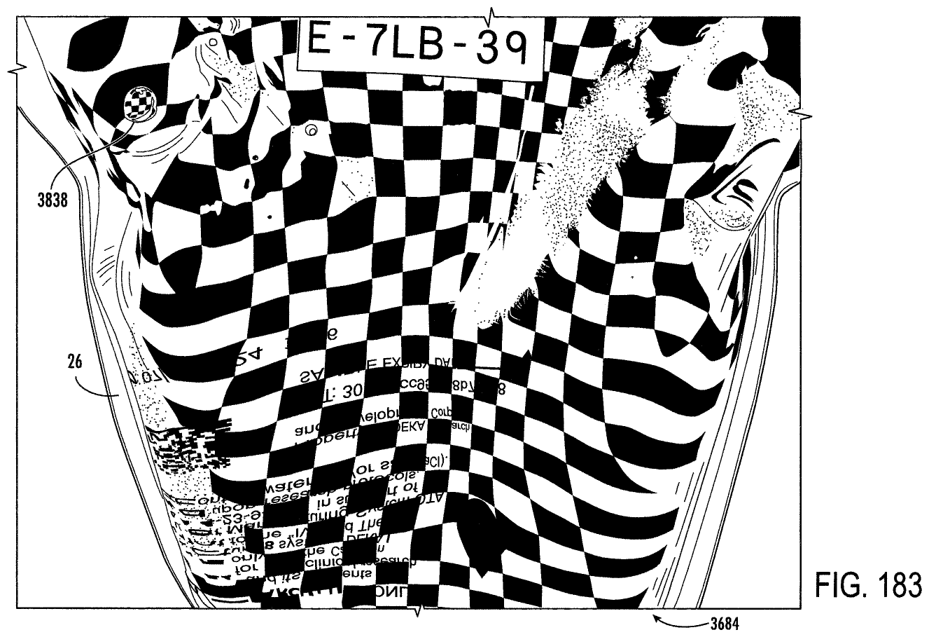

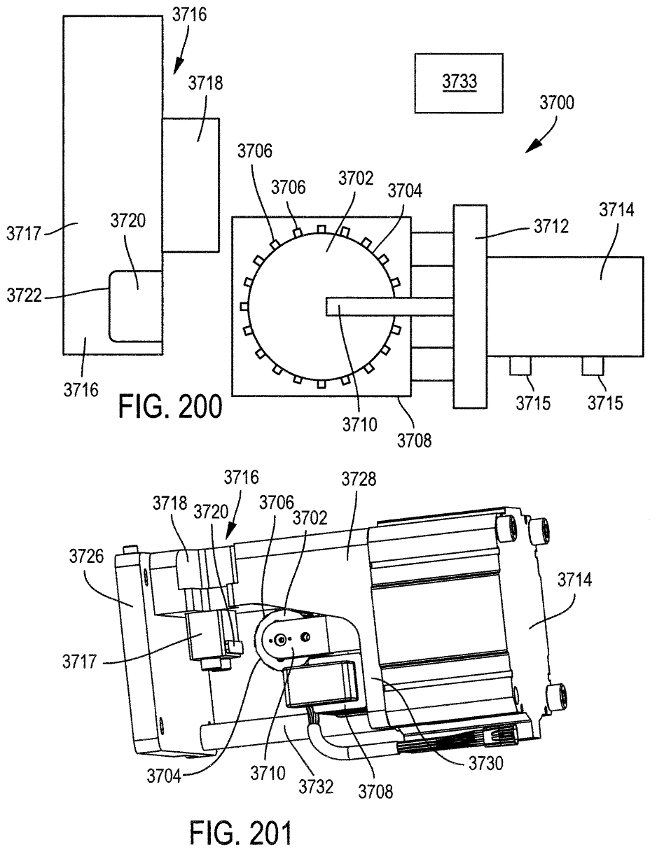

The control system 15 may command the rotary actuator 3470 to displace the bag 26 with the doors 3400A, B actuated closed upon the bag 26. In some embodiments, the control system 15 may command displacement of the bag 26 as described in relation to FIG. 158 and may mix fluid within the bag 26. The bag 26 may be returned to a starting position and the doors 3400A, B may be opened to provide a clear field of view for the imagers 3518. After a wait period has elapsed (e.g. 5-10 seconds) the control system 15 may command the imager(s) 3518 to capture frames of the bag 26. Depending on the embodiment, frames may be captured at a rate of at least 10 frames per second (though higher or lower capture rates are possible). Frames may be captured for at least 10 seconds (though capture periods greater than or less than 10 seconds are also possible). The control system 15 may process the captured frames from each imager 3518 to determine the presence of one or more contents of interest in the bag 26. In some examples, the control system 15 may include an FPGA or dedicated processor for this task. The control system 15 may create an inspection log 3590 (see, e.g., FIG. 179) which may be communicated to a database 3502 or other memory. Each inspection log 3590 may include at least one of the raw frames 3684 (see, e.g., FIG. 183) from the imagers 3518, analysis results for the frames (type of content identified, number of particles, size of particles, location of particles, etc.), information about the bag 26 (e.g. lot, unique identifier, solution type within bag 26), and a pass/fail determination for the bag 26. The system 10 may ensure that bags 26 which are determined to have failed inspection criteria are routed to a discard or quarantine destination after leaving the particulate inspection system 3500. The system 10 may additionally ensure that such bags 26 are clearly labeled as rejected (see, e.g., FIGS. 200-212B and associated description). The control system 15 may also save the inspection log 3590 (or at least some information from the inspection log 3590) as part the batch record for the inspected bag 26. Any information in the inspection log 3590 may be included in a batch record.

As shown in FIG. 159, a particulate inspection system 3500 may also include an indicia reader 3543 for an indicium 56 on the bag 26. The indicia reader 3543 may be an imager, barcode reader, QR code reader, RFID interrogator, NFC interrogator, etc. In certain examples, the bag 26 may include a GS-1 code as the indicium 56 and the indicia reader 3543 may be an imager. In alternative embodiments, one or more of the imagers 3518 may be utilized as the indicia reader 3543 and a dedicated indicia reader 3543 may be omitted. The control system 15 may determine at least a unique identifier for the bag 26 from data captured by the indicia reader 3543 (or one or more imager 3518) and may associate any particulate inspection records with that unique identifier when records are communication to a database 3502. In some embodiments, such an indicia reader 3543 may also be disposed in the processing compartment 4300. The indicia reader 3543 may determine various information from the indicium 56 (e.g. concentrate type, volume, mass, etc.) which may be used to inform dispensing of fluid into the bag 26 from a filling station 2600. Any information read from the indicium 56 on the bag 26 may also be written to the batch record for that bag 26 by the control system 15.

Referring now to FIG. 160A-C, a number of illustrations of an example particulate inspection system 3500 are depicted. The example particulate inspection system 3500 includes the mix assisting assembly 2500 depicted in FIGS. 155A-156B. As shown, the rest body 3510 includes a checker pattern or repeating dark and light regions 3514A, B. This pattern, though distorted by the bag 26 (see, e.g., FIG. 160B), may be visible through the bag 26 from perspective of the imagers 3518. The light and dark regions 3514A, B may be an applique which is placed on the rest body 3510 (e.g. the exterior surface of the rest body 3510). In alternative examples, the pattern may be generated on the rest body 3510 by, hydro dip painting, stenciling, screen printing, or laser etching the pattern into a painted rest body 3510. Laser etching may also be used to generate roughness in a pattern on a rest body 3510 which is otherwise too smooth for paint to adhere. The rest body 3510 would then be painted and the paint may be removed from the unetched surface. A photoreactive coating could also be applied, exposed to light, and developed to generate the desired pattern.

The particulate inspection system 3500 may include a mount assembly 3520 to which the imager(s) 3518 may be coupled. The mount assembly 3520 may be coupled to the enclosure 12 and may ensure that the imagers 3518 are positioned at a prescribed angle (e.g. 32°) to the medial transverse plane of a bag 26. As shown, each of the imagers 3518 may be associated with a filter 3542. In various embodiments, the filter 3542 may be neutral density filter. Alternatively or additionally, a polarizer 3517 may be associated with each imager 3518 (see, e.g., FIG. 159).

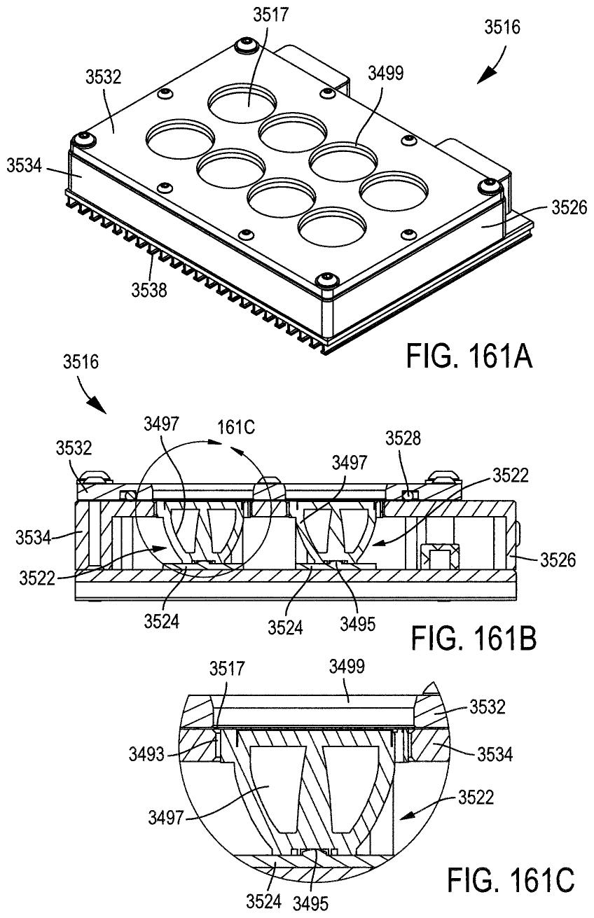

The external illuminator 3516 is also depicted in FIGS. 160A-C. Another example is depicted in isolation throughout FIGS. 161A-C. As best seen in FIG. 160C and FIG. 161B, the external illuminator 3516 may include a plurality of light emitter assemblies 3522. Each light emitter assembly 3522 may include an LED 3495 and collimating lens or narrow beam angle lens 3497. The LED's 3495 and PCB 3524 to which they are coupled may be disposed within a housing 3526. The housing 3526 may include one or a series of sealing members 3528 (e.g. o-rings) which may inhibit ingress of any liquid into the housing 3526. The sealing member(s) 3528 may be formed of a compliant material which may be compressed by a face member or plate 3532 coupled to a main section 3534 of the housing 3526. This may facilitate placement of the external illuminator 3516 under the bag 26 within a particulate inspection assembly 3500. The external illuminator 3516 may also include a polarizer 3517. The polarizer 3517 may form part of the housing 3526 and span across a light emission aperture 3536 of the housing 3526. Alternatively, multiple light emission apertures 3536 may be included and may be spanned by a polarizer 3517. This may help to leverage the polarizer 3517 as a retainer for the lenses 3497 associated with each LED 3495. The main body 3534 may include receiving apertures 3493 for an emitting end of each of the lenses 3497 which may further assist in preventing displacement of the lenses 3497. Where multiple light emission apertures 3536 are included, the apertures may include a tapered region 3499 which increases the light emission aperture 3536 width as distance from the respective lens 3497 increases. This may ensure that the light emitted from the light emitter assemblies 3522 is substantially unblocked by the housing 3526.

A heat sink 3538 may be coupled to the housing 3538 to assist in dissipating heat generated by the light emitter assemblies 3522. The heat sink 3538 (or a base intermediate the heat sink 3538 and remainder of the housing 3526) may include guides which ensure that the main portion 3534 of the housing 3526 is aligned with the light emitting assemblies 3522 during assembly. The housing 3526 may include a mount 3540 via which the external illuminator 3516 may be fixed in place relative to the imagers 3518. The mount 3540 may, for example, couple to the enclosure 12 (see, e.g., FIG. 159).

Depending on the layout of the array of individual light emitting assemblies 3522 in an external illuminator 3516, the angle of the light with respect to the height axis of the bubble 3550 may change. In the example shown in FIG. 160C for instance, the light emitting assemblies 3522 of the external illuminator 3516 are arrayed in two rows. The collimated light from these rows may be generally directed at the midplane of a bag 26 retained in the particulate inspection system 3500. To accomplish this, the light emitting assemblies 3522 may be positioned to emit light at an angle non-parallel to the midplane. For certain external illuminators 3516, the light emitting assemblies 3522 in each row of the array may be spaced an even amount from respective opposing sides of the midplane. The light emitting assemblies 3522 may emit collimated light at an angle of 15° to the midplane with each row tilted in opposite directions. In other embodiments with additional rows or different light emitting assembly 3522 array layouts, the light emitting assemblies 3522 may be angled to emit collimated light at a center point or central plane of a bag 26.

Alternatively, the narrow beam angle engendered by the lenses 3497 of each light emitting assembly 3522 (see, e.g., FIGS. 161A-C) may ensure that the entire bag 26 is bathed substantially uniformly in light from the external illuminator 3516 when the external illuminator 3516 is powered. The light emitting assemblies 3522 may not be oriented other than substantially normal to the base of the external illuminator 3516.

The light emitter assemblies 3522 may be wired in series in certain examples (though this is not necessary in all embodiments). With such an arrangement, the failure of one light emitter assembly 3522 would result in failure of the entire external illuminator 3516. This prohibits a scenario in which one or more light emitter assembly 3522 fails and is not detected. Thus, the external illuminator 3516 may be ensured to operate at only 100% output. Detection of a light emitter assembly 3522 failure would also be simplified as no light would be generated when the external illuminator 3516 is commanded on.

In some embodiments, a photodetector in data communication with the control system 15 may be positioned opposite the external illuminator 3516. In the event the signal output by the photodetector is less than a threshold, the control system 15 may generate a fault. Alternatively, image data from the imagers 3518 may be utilized to detect a failure of the external illuminator 3516. The control system 15 may, for example determine an overall light intensity value for the field of view of an imager 3518 and may generate a fault if the field of view is below a darkness threshold when the external illuminator 3516 is commanded on. In some embodiments, the control system 15 may additionally or instead determine failure of the external illuminator by analyzing the color space values for image data from an imager 3518. The control system 15 may compare the image data to expected color space value ranges (e.g. RGB color space). If the external illuminator 3516 emits red light, for instance, a high R value and lower G and B values would be expected to be observed in at least portions of an image In the event that the color space values observed are unexpected (e.g. substantially uniformly low RGB values) the control system 15 may generate a fault. Alternatively, a neural net may be trained with images where the external illuminator 3516 is powered and verified to be functional. The neural net would also be trained with images taken when the external illuminator 3516 is unpowered and in a non-illuminating state. An image may periodically be supplied for neural net analysis to check for failures of the external illuminator 3516. A fault may be generated by the control system 15 in the event of a determination by the neural network that the external illuminator 3516 has failed. Where a fault is generated, the control system 15 may generate a notification for display on the user interface 6100 (see, e.g., FIG. 1) communicating the external illuminator 3516 failure.

Referring now to FIG. 162, a flowchart 3450 detailing a number of exemplary actions which may be executed to verify a particulate inspection system 3500 is functioning as expected is depicted. As shown, in block 3451 the control system 15 may command image capture from each of the imagers 3518 of the particulate inspection system 3500. If, in block 3452, no imagers 3518 are operational (e.g. connections cannot be established) or if, in block 3454 an imager outputs an image which is not valid (e.g. blank or all black) a fault notification may be generated in block 3453. If, in block 3452, at least one imager 3518 is operational and operational imagers 3518 output valid images in block 3454, the functionality check may continue. Proceeding even with non-operational imagers 3518 is optional, though may be preferred as it may generate more information for service personnel tasked with troubleshooting the particulate inspection system 3500. In such instances, images may be captured from any operational imagers 3518 which are generating valid images and any image analysis may be limited to data from imagers 3518 which are determined to be functioning properly.

As shown, in block 3455, images from each imager 3518 may be aligned with respective first reference images by a processor of the control system 15. The images captured by the imagers 3518 for comparison against reference images are referred to as test images in relation to FIG. 162. The first reference images may be specific to each imager 3518 in the particulate inspection system 3500 and may be captured during set up or by service personnel after a particulate inspection system 3500 is verified to be functioning appropriately. The image registration or alignment may be performed by matching points on the image captured by an imager 3518 to one or more corresponding reference point in the reference image for that imager 3518. This could for example be a feature (e.g. a point where corners of a number of squares of a chessboard pattern meet) of the background pattern on the rest body 3510 for example. A perspective transformation may then be performed to support a comparison of the reference images with the respective test images. If, in block 3456, the images do not meet predefined quality criteria, a fault notification may be generated in block 3453.

In various examples, the images captured in block 3455 may be compared with a pixel by pixel comparison, feature based matching, a trained neural network, or any other suitable alternative. In certain embodiments, a mean square error comparison may be conducted by the control system 15. In the event the mean square error comparison indicates a difference in excess of a predefined threshold, the quality criteria may be failed. If the threshold is exceeded for the comparison of any one test image to the respective reference image, the quality criteria may be deemed failed. The output of the mean square error analysis may be in excess of the threshold in the event that the focus of the test images is unacceptable, occlusions (e.g. detritus/liquid on imager 3518 lens, or rest body 3510 including the background pattern) are present, or other distortion of the test images is present. Though examples described in relation to FIG. 162 use a mean square error analysis, other analysis or analyses may be used instead or in combination with a mean square error analysis in alternative embodiments.

If, in block 3556, the quality criteria is met, the control system 15 may command the backlight (e.g. illuminator 3512) to be powered in block 3457. With the backlight powered, an image from each imager 3518 (or operational imager 3518) may be collected in block 3458. These test images may be aligned or registered against a second set of respective reference images for each imager 3518 in block 3458 as well. The second reference images may be captured during service or setup of a particulate inspection system 3500 when the backlight is verified to be operating as expected by a technician or service personnel. If, in block 3459, analysis of the images indicates that the backlight is not emitting light as expected, a fault notification may be generated in block 3453. In certain examples, a mean square error analysis may, for example, be executed by the control system 15 using the second reference images and the test images from block 3458. If the difference output by the mean square error analysis is in excess of a threshold, the comparison may be deemed to have failed. The threshold may be breached in the event that the colors and/or light intensity in the test images does not correspond to the respective reference images. If the comparison of any one test image to the respective reference image exceeds the threshold, it may be determined the backlight is not functioning as expected.

If, in block 3459, the analysis determines that the backlight (e.g. illuminator 3512) is functioning as expected, an external illuminator 3516 may be commanded on by the control system 15 in block 3460. The backlight may typically be turned off, though need not necessarily be in all embodiments. With the external illuminator 3516 powered, an image from each imager 3518 may be collected in block 3461. These images may also be aligned with respective third reference images in block 3461. As with other reference images, the third references images may be captured by a technician or service personnel after verification that the external illuminator 3516 and any other powered lighting sources are working properly. If, in block 3462, the analysis by the control system 15 indicates the external illuminator 3516 is not emitting light, a fault may notification may be generated in block 3453. In certain examples, a mean square error analysis may, for example, be executed by the control system 15 using the third reference images and the test images from block 3461. If the difference output by the mean square error analysis is in excess of a threshold, the comparison may be deemed to have failed. The threshold may, for instance, be breached in the event that the colors and/or light intensity in the test images does not correspond to the respective reference images. If the comparison of any one test image to the respective reference image exceeds the threshold, it may be determined the external illuminator 3516 is not functioning as expected.

If, in block 3462, the comparison between the third reference images and the images captured in block 3461 indicates the external illuminator 3516 is emitting light as expected, a reservoir may be installed in the particulate inspection system 3500 in block 3463. Optionally, images may be collected from each imager 3518 after the reservoir is retained in the particulate inspection system 3500 in block 3464. These images may be utilized to verify a reservoir is properly retained in the particulate inspection system 3500. Alternatively, the doors 3400A, B of the particulate inspection system 3500 may be closed and the position of the doors 3400A, B may be monitored by the control system 15 to verify a reservoir is in place at the particulate inspection system 3500. Where reservoir installation is verified via imaging, the collected images may be aligned against reference images in block 3464 as well. The reference images used in block 3464 may be fourth reference images or the second or third reference images in some examples. Where the second reference images are used, the backlight (e.g. illuminator 3512) may be powered when the images of block 3464 are taken. Where the third reference images are used, the external illuminator 3516 may be powered when the images of block 3464 are taken. Where fourth reference images are used, both the backlight and external illuminator 3516 may be powered when the images of block 3464 are taken (no reservoir would be present). The fourth reference images may be captured by service personnel when the backlight and external illuminator 3516 are verified to be functioning as expected.

If, in block 3465, comparison of the images from block 3464 to the appropriate reference images indicates that a reservoir is absent, a fault notification may be generated in block 3453. In certain examples, a mean square error analysis may, for example, be executed by the control system 15 using the test images from block 3464 and the appropriate reference images. If the difference output by the mean square error analysis is in below a threshold, the comparison may be deemed to have failed. The threshold may be breached in the event that the test images closely match the respective reference images for each imager 3518. This would indicate that no reservoir is in place and the test images are of an empty particulate inspection system 3500. If the comparison of any one test image to the respective reference image is in breach of the threshold, it may be determined the reservoir is not properly retained.

If, in block 3465, the comparison indicates a reservoir is present, the reservoir may be agitated and inspected in block 3466. In some examples, the functionality test may be performed before each reservoir is inspected. A record of the functionality test may be included in the log file 3590 (see, e.g., FIG. 179) associated with each reservoir. In the event, that lighting components or imaging components are determined not to be functioning as expected, the reservoir may be indicated to have not passed inspection. The reservoir may be directed to a quarantine or access controlled repository where the particulate inspection system 3500 is part of a larger system. The parent system 10 may also prohibit filling of any further reservoirs until the particulate inspection system 3500 has been serviced.

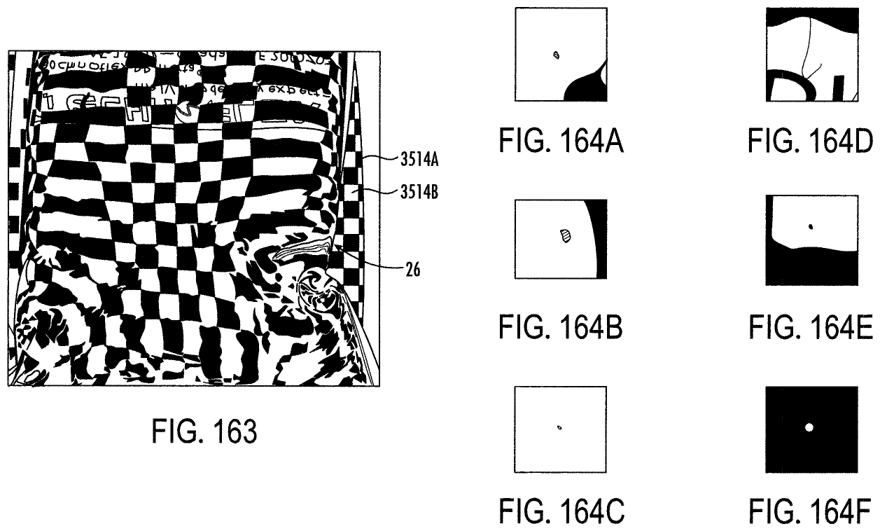

Referring now to FIG. 163, a portion of an IV bag 26 against a backlit pattern 20 that extends at least across the entire width of a bag 26 is depicted. Various content types in a bag 26 are shown in enlarged representations of images across FIGS. 164A-F. FIG. 164A depicts an enlarged portion of a representation of an image of a particle of an example septum for a bag 26 against a light region 3514B of a background pattern. FIG. 164B depicts an enlarged portion of a representation of an image of a particle of poly(methyl methacrylate) (PMMA) material (highly transparent thermoplastic) against a light region 3514B of a background pattern. FIG. 164C depicts an enlarged portion of a representation of an image of a particle of glass against a light region 3514B of a background pattern. FIG. 164D depicts an enlarged portion of a representation of an image of a fiber against light and dark regions 3415A, B of a background pattern. FIG. 164E depicts an enlarged portion of a representation of an image of a particle of black material against a light region 3514B of a background pattern. FIG. 164F depicts an enlarged portion of a representation of an image of a bubble 3550, illuminated by an example particulate inspection system 3500, against a dark region 3514A of a background pattern.

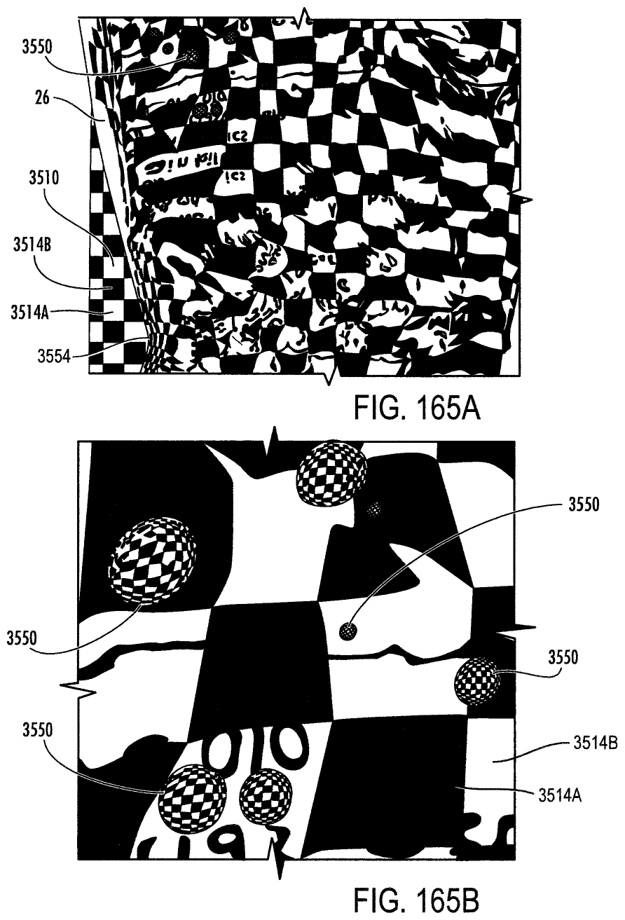

Referring now to FIGS. 165A-175D, a particulate inspection system 3500 may differentiate between certain contents of interest within a bag 26. Bubbles 3550 of air or gas may be commonplace within bags 26, especially after the bag 26 has been agitated by a mix assisting assembly 2500. Bubble 3550 content should be benign and the presence of bubbles 3550 alone preferably would not trigger a bag 26 to fail inspection. The external emitter 3516, background pattern, and imager(s) 3518 may be selected, outfitted, and positioned to help ensure bubbles 3550 within the solution contained in a bag 26 are readily differentiable from other contents of interest.

Referring to FIGS. 165A-B, illustrations of portions of a bag 26 in place at a cradle 3311 of a particulate inspection system 3500 are depicted. The illustrations are representative of what would be seen from an imager 3518, but not exact reproductions of actual images. A representational frame is depicted in FIG. 165A. An enlarged portion of the representational frame of FIG. 165A is depicted in FIG. 165B. The dark and light regions 3514A, B of the rest body 3510 pattern are also visible in FIGS. 165A-B. As shown, the bag 26 may distort this pattern as light from the back light transits through the bag 26. A portion of that pattern unadulterated by passage through bag 26 is visible lateral to a crinkle or fold 3554 in the side of the bag 26 in FIG. 165A.

Still referring to FIG. 165A-B, a number of large bubbles 3550 are present in the bag 26 and positioned against the wall of the bag 26 material. As shown, the optical properties of the bubbles 3550 distorts the backlight pattern in a similar manner for each of the bubbles 3550. Each of the bubbles 3550 creates a distorted version of a section of the pattern. In the example, the pattern is a chessboard pattern and a distorted checkered pattern is visible in each of the bubbles 3550. Bubbles 3550 suspended in solution may also distort the background pattern in a consistent manner. As the distorted versions of the background pattern are hallmarks indicating the existence of bubbles 3550, images may be analyzed for the presence of these specific patterns in order to identify the presence of bubbles 3550 within a bag 26. The control system 15 may employ a pattern recognition algorithm such as a statistical pattern recognition algorithm or syntactic pattern recognition algorithm. Template matching may for example be used to match instances of the distorted background pattern to an expected distorted background pattern template. A neural network may also be trained on images of bubbles 3550 creating the distorted checkered pattern and employed to detect the presence of such bubbles 3550 based on presence of the pattern.

The resolution of the imager 3518 capturing the image frame limits the size of bubble 3550 which may be identified by the presence of the distorted background patterns. Higher resolution imagers 3518 may detect smaller bubbles 3550 by analyzing frames for the presence of the distorted background pattern. In some embodiments, recognition of the distorted background pattern may be used to differentiate relatively large volume bubbles 3550 from other large contents of interest.

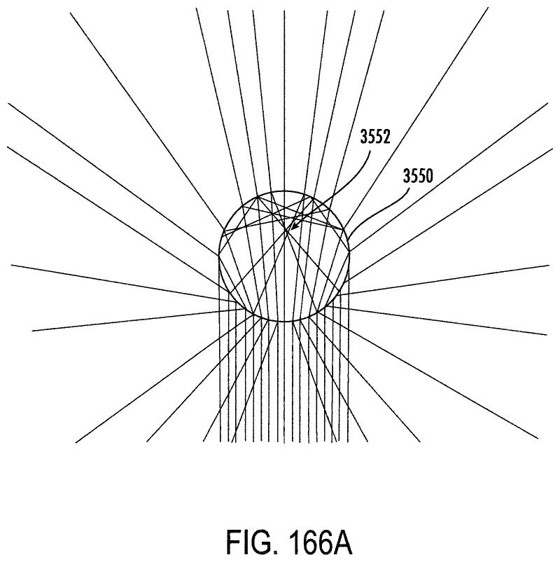

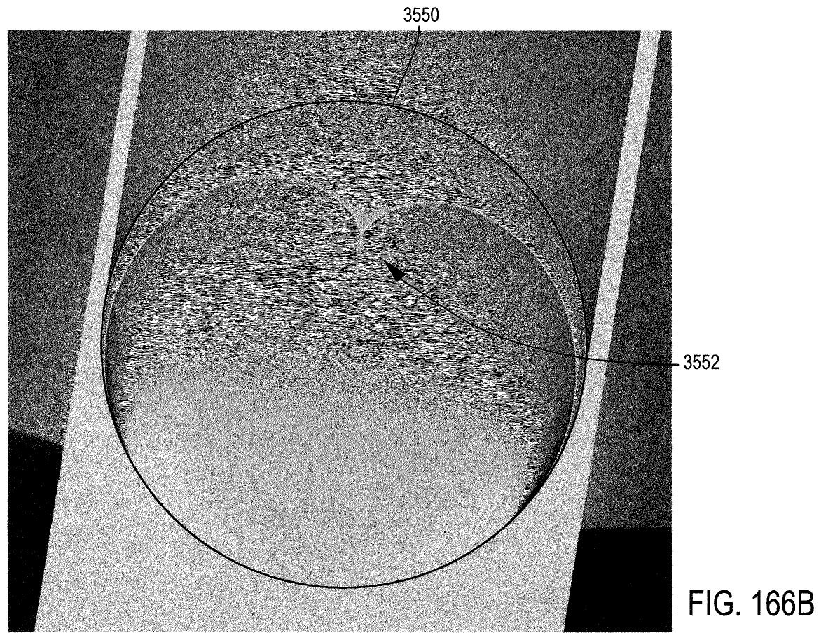

Referring now to FIGS. 166A-B, bubbles 3550 may be detected based on the manner in which they interact with light emitted from an external illuminator 3516. This may be particularly useful for identifying small bubbles and differentiating them from other similarly sized contents of interest. Representational diagrams of bubbles 3550 are depicted in FIGS. 166A-B. In general, due to the constant and evenly distributed pressures on the inside and outside of the bubbles 3550, a bubble 3550 takes on a shape which minimize its surface area. Thus, bubbles 3550 are typically spherical. Other contents of interest, however, have a very low likelihood of being as close to spherical as any bubbles 3550 present in a bag 26. The optical properties bestowed by the spherical shape of a bubble 3550 may be exploited to cause bubbles 3550 to have a consistent and highly perceptible appearance when imaged by a particulate inspection system 3500.

FIG. 166A depicts an example illustrative diagram of a bubble 3550 being illuminated with collimated light (e.g. from an external illuminator 3516 of the type described in relation to FIG. 159). The light may approach the bubble 3550 in collimated manner as shown. As mentioned elsewhere herein, the light may also be polarized. As required by Snell's Law, the path of the light will be perturbed by the bubble 3550. An illustration based on a computer simulation of the same is provided in FIG. 166B. The collimated light is directed at the bubble 3550 at an angle of 15° in FIG. 166B.

As shown, in each of FIGS. 166A-B, the light reflected within the bubble 3550 may be concentrated at a focused region 3552. Other interactions with the bubbles 3550 may further lead the bubbles 3550 to create bright points of light within the bag 26 (further elaborated in relation to FIG. 168 for example). These bright points of light may be particularly discernable when viewed from certain angles (e.g. 30-35° inclined from an axis perpendicular to the bag 26 midplane). Bright points engendered by bubbles 3350 may be significantly brighter than other contents of interest within the bag 26. The color emitted by the external illuminator 3516 may also be selected to be different from that of any potential anticipated source of particulate. Thus, not only will bubbles 3550 generate bright points within a bag 26, these points may be in a known, predefined, and controllable in color. The color preferably is selected to be a color which would not be expected from any other contents of interest with a potential to be present in the bag 26. Bright points may be generated regardless of the volume of a bubble 3550. Large volume bubbles 3550 may generate bright points of light as well as very small microbubbles (e.g. 50-100 μm diameter bubbles 3550 or smaller).

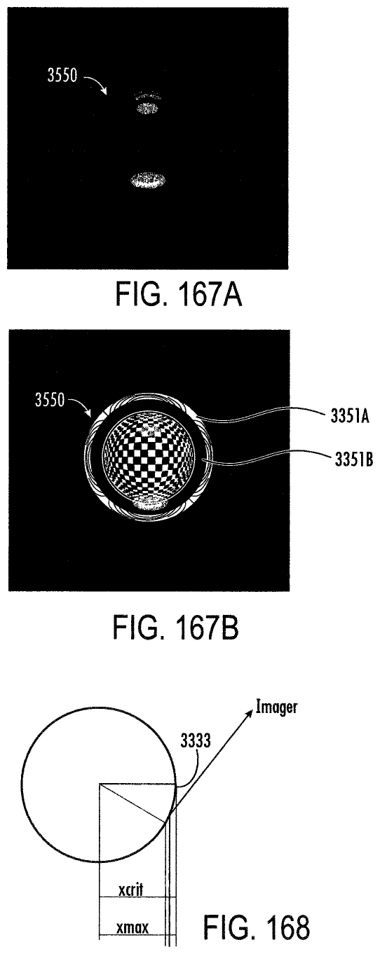

Referring now primarily to FIGS. 167A-B, a bubble 3550 may be readily distinguishable from other content types by the large amount of light reflected by the bubble 3550. FIGS. 167A-B both simulate a bubble 3550 in a filled bag that reflects high-intensity light projected from the bottom of the page. FIGS. 167A-B are from a vantage point where the line of sight extends along the medial transverse plane of the bubble 3550 and through the center of the bubble 3550. FIG. 167A is demonstrative of a bubble 3550 illuminated from below with no background pattern present. As shown in FIG. 167B, reflection and refraction of a checkered pattern (e.g. backlit background pattern) is also shown. Refraction can be seen at the central region of the bubble 3550. A ring 3351A of reflection of background may surround a dark ring 3351B. The dark ring 3351 may be due to total internal reflection (T.I.R.) of light from the background pattern in this region of the bubble 3550. These rings 3351A, B may assist in distinguishing bubbles 3350 from other content types. The stippling generally depicts regions of the particle 3505D where light from an underlight type external illuminator 3516 is perceptible. The density of the stippling generally approximates the brightness of this light.

As illustrated by the diagram in FIG. 168, a bubble 3550 reflects up to 100% of incident light at angles greater than the critical angle. This is due to the index of refraction of the bubble 3550 being less than that of the surrounding medium. As shown best in FIG. 168, a ray of light reaching the bubble 3550 in a region 3333 where the angle of incidence is greater than the critical angle will reflect entirely off the surface of the bubble 3550. Consequently, reflected light from a bubble 3550 can exhibit an intensity that approaches the intensity of the light emitted by an external illuminator 3516 of FIG. 160C for instance.

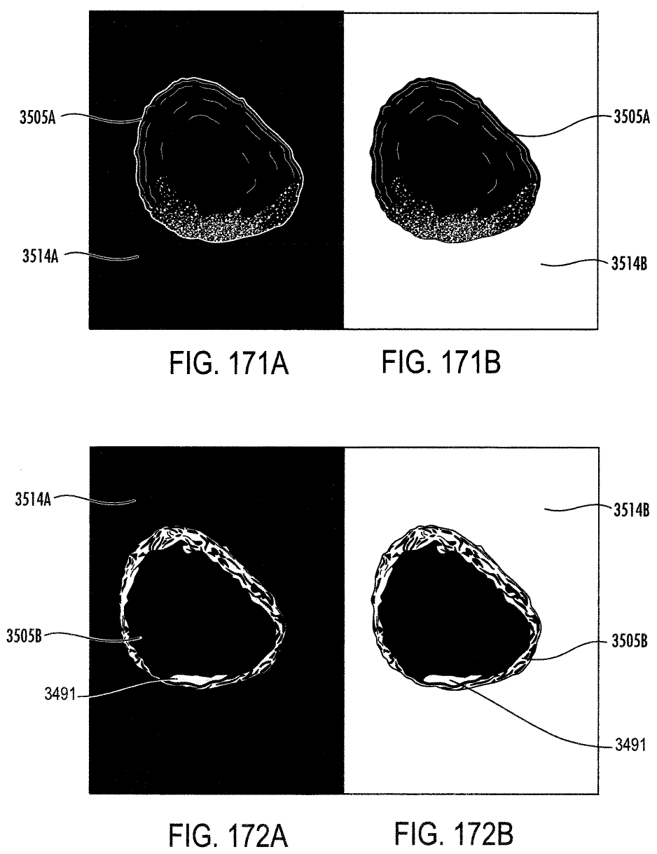

In contrast, typical particulate (see, e.g., FIGS. 171A-174B), for example, tends to reflect only a fraction of incident light resulting in a reduced light intensity. This may be utilized to distinguish such particulate from bubbles 3350 even if the bubbles 3350 are particularly small (e.g. less than 200 μm in diameter). The maximum luminance of a particle with Lambertian surfaces may be approximated by the equation L=(ρ*E)/π. where ρ is the reflectivity of the material and E is the illuminance of light on the surface in lux.

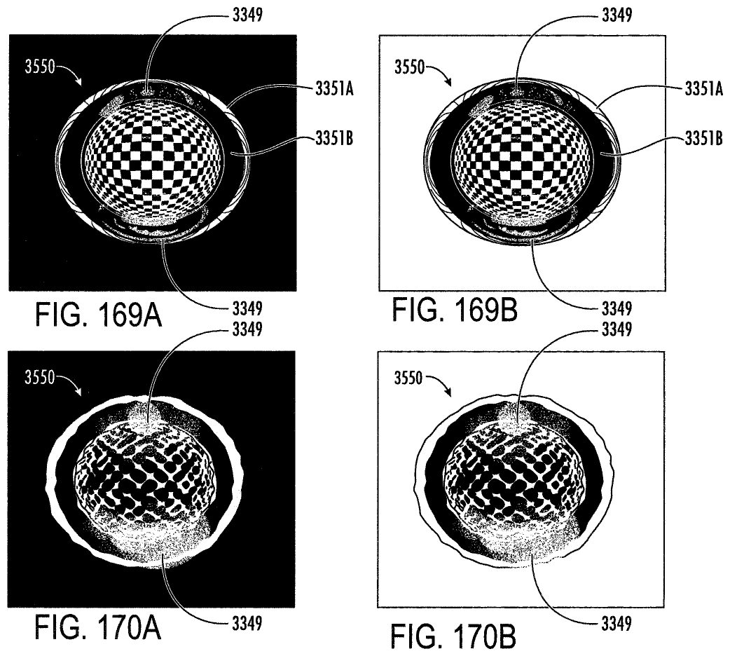

Referring to FIGS. 169A-170B, the amount of light directed to the imagers 3518 as a result of the optics of a bubble 3550 (in a medium with a higher index of refraction) allows bubbles 3350 to be robustly distinguished from other content types. It may additionally facilitate detection of bubbles 3350 in a subject reservoir which has a depth in excess of the crisp depth of field range for an imager 3518. Despite being out of focus, the bubble 3550 would remain abundantly visible. As depicted in the representations of simulated images in FIGS. 169A-B crisply in focus bubbles 3550 provide not only a high intensity bright regions 3349, but also a distorted background pattern and characteristic rings 3351A, B. If out of focus, a bubble 3550 will still be clearly observed due to the high intensity bright regions 3349 captured in images from imagers 3518. FIGS. 170A-B depict illustrative representations of simulated images of bubbles 3550 which are not in crisp focus. The high intensity bright regions 3349 and other stippling are due to interactions of the bubble 3550 with light emitted by an underlight type external illuminator 3516. The density of the stippling in FIGS. 169A-170B generally approximates the brightness of the light reaching the imager 3518 from the external illuminator 3516.

Referring to FIGS. 171A-B, a representation of a simulated image of a particle of opaque white material 3505A is shown respectively against a dark region 3514A (FIG. 171A) and a light region 3514B (FIG. 171B) of a background pattern (e.g. a chessboard pattern). As mentioned elsewhere herein, these representations, and others described herein, are not exact reproductions of actual images and are provided for illustrative purposes. The particle 3505A is simulated in a medium with an index of refraction of 1.33 (e.g. water) and itself has an index of refraction of about 1.49. The particle of opaque white material 3505A is illuminated from below by at least one light source (e.g. an external illuminator 3516) and a second light source (e.g. backlight illuminator 3512) is illuminating the background pattern. As shown, the particle of opaque white material 3505A may be clearly visualized against both light and dark regions 3514A, B of a background pattern. The stippling generally depicts regions of the particle 3505A where light from an underlight type external illuminator 3516 is being reflected. The density of the stippling generally approximates the brightness of the reflection of light emitted by the external illuminator 3516.

Referring now to FIGS. 172A-B, particles of reflective materials, for example, those with high albedo values (0.5-1) may also be discernable against both regions 3514A, B of a background pattern. Various metallic materials for example may be highly reflective. FIGS. 172A-B depict a simulation of an aluminum particle 3505B in a gas. The periphery of the particle 3505B displays a distortion of the background pattern with the center of the particle appearing dark. Additionally, a strong reflection 3491 of light from an underlight type external illuminator 3516 is present at the bottom of the particle 3505B.

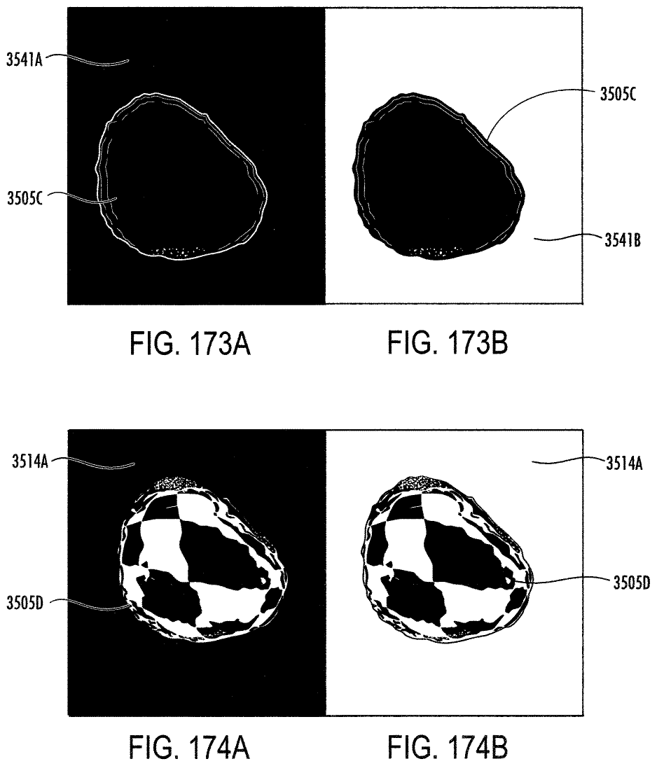

Referring now to FIGS. 173A-B, a representation of a simulated image of an illustrative particle 3505C which is fully opaque (to visible light) and which strongly absorbs visible light shining thereon is depicted. The particle 3505A is simulated in a medium with an index of refraction of 1.33 (e.g. water) and itself has an index of refraction of about 1.49. This illustrative particle 3505C may be strongly discerned as it passes across light regions 3514B of the background pattern, but also is perceptible to a degree in front of dark regions 3514B. The stippling generally depicts regions of the particle 3505C where light from an underlight type external illuminator 3516 is being reflected. The density of the stippling generally approximates the brightness of the reflection of light emitted by the external illuminator 3516.

Referring now to FIG. 174A-B, a representation of a simulated image of an illustrative particle 3505D of transmissive material is depicted. Transmissive materials have less than total opacity, passing at least some light. The example shown is a clear particle 3505D of quartz in water. As shown, such particles 3505D may be clearly discerned in front of dark and light regions 3514A, B of the background pattern and generate a refracted region of distorted background pattern. The stippling generally depicts regions of the particle 3505D where light from an underlight type external illuminator 3516 is perceptible. The density of the stippling generally approximates the brightness of this light.

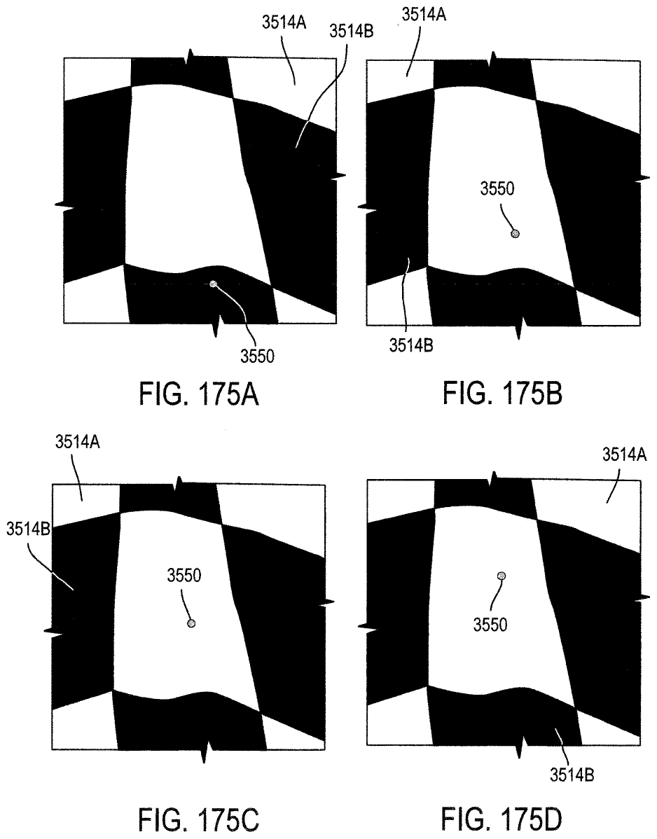

Referring now primarily to FIGS. 175A-D, a number of illustrations representative (though not exact reproductions) of portions of a bag 26 seen from an imager 3518 are depicted. Each of the illustrations depicts a bubble 3550 illuminated by an external illuminator 3516 such as that shown in FIG. 160C. The progression of FIGS. 175A-D depict the bubble 3550 displacing through the bag 26 due to, for example, buoyancy and/or agitation of the bag 26. The bubble 3550 is depicted as a grey circle which is representative of the bright light points described above. As shown, the bubble 3550 is readily distinguishable against light regions 3514A and dark regions 3514B of the background pattern.

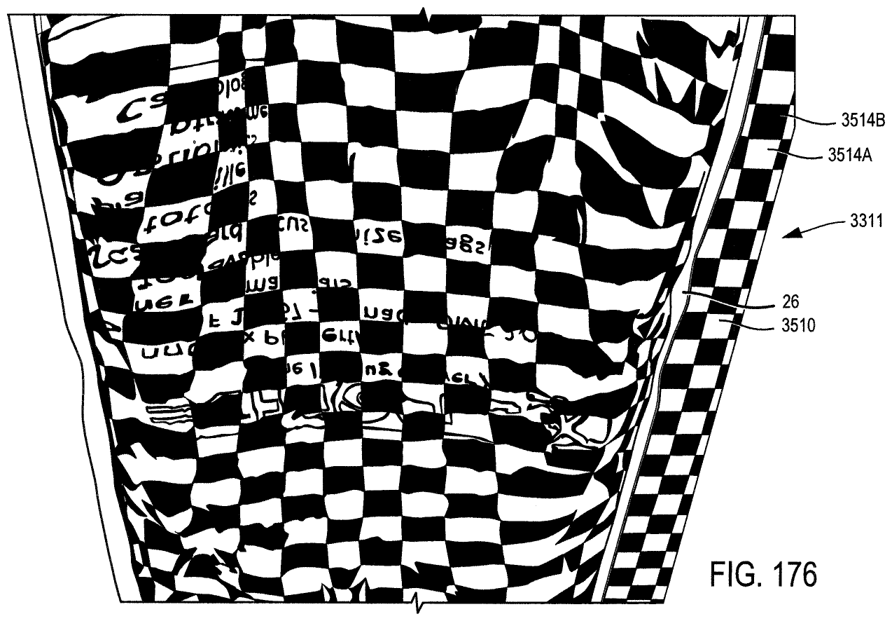

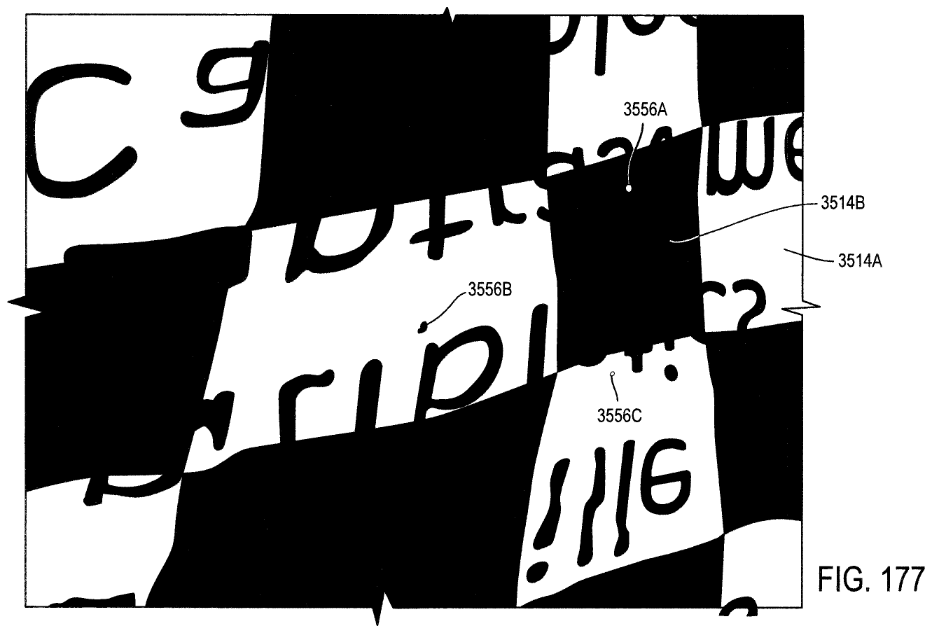

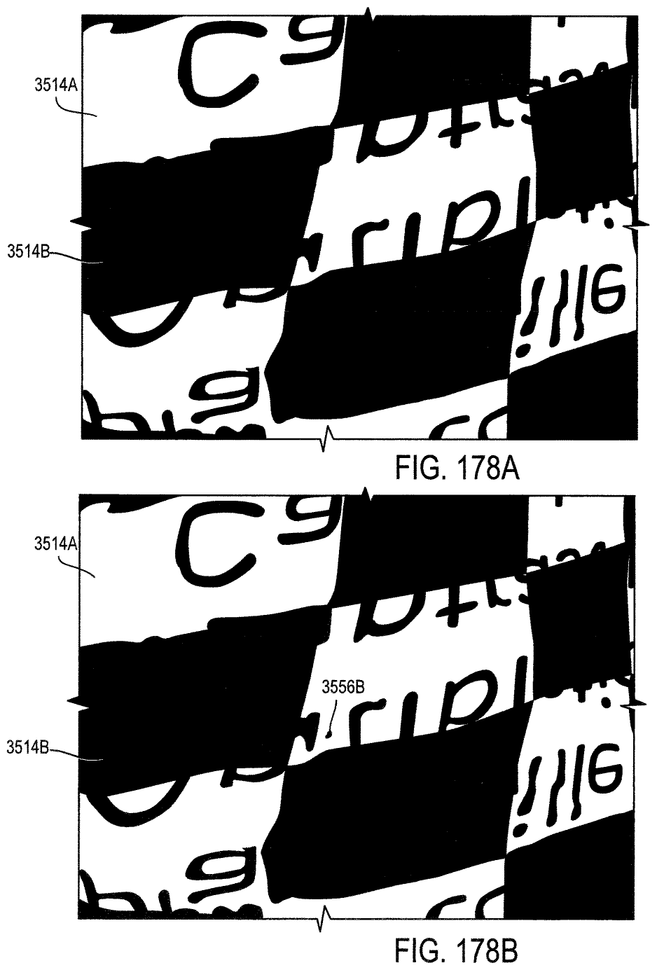

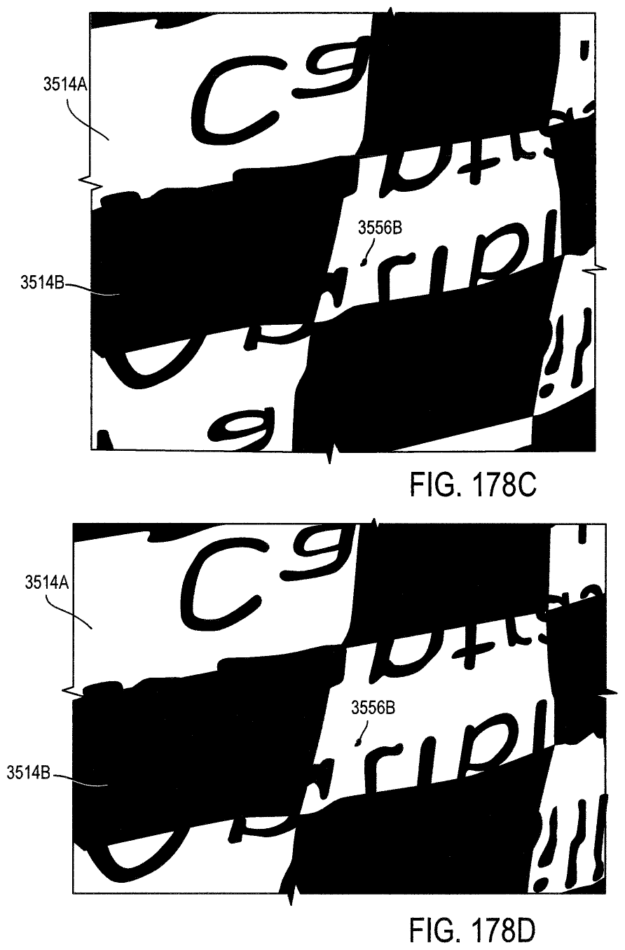

Referring to FIGS. 176-178D, further illustrations of portions of a bag 26 in place at a cradle 3311 of a particulate inspection system 3500 are depicted. The illustrations are representative of what would be seen from an imager 3518, but not exact reproductions of actual images. A representational frame is depicted in FIG. 176. An enlarged portion of the representational frame of FIG. 176 is depicted in FIG. 177. The dark and light regions 3514A, B of the rest body 3510 pattern are also visible in FIGS. 176-177. FIGS. 178A-D depict the portion of FIG. 176 shown in FIG. 177 over a period of time with an illustrative piece of particulate present (other particles in FIGS. 176-177 have been removed in FIGS. 178A-D).

Referring specifically to FIG. 176-177, a number of pieces of particulate 3556A-C of differing colors are shown. For sake of illustration white particulate 3556A, black particulate 3556B, and clear particulate 3556C are depicted. These particulate colors are selected as worst case colors in order to illustrate various advantageous aspects of example particulate inspection systems 3500 described herein. Colors of particulate from potential particulate sources in a system 10 may have greater variety and may not include the colors shown.

As shown, the backlight pattern (or a backlight otherwise controlled to generate contrast, e.g., temporally) may facilitate visualization of various pieces of particulate 3556A-C as they displace within the bag 26. This may be true regardless of the color of a particular piece of particulate 3556A-C. Prior to capturing frames with the imagers 3518 of the particulate inspection system 3500, the rotary actuator 3470 (see, e.g., FIG. 160C) may be commanded to rotate the cradle 3311 and bag 26 through an agitation sequence or motion profile. The agitation sequence may, in some examples, be a series of rotations as described in relation to FIG. 158. In such embodiments, mixing of the fluid within the bag 26 may also serve to agitate the fluid within the bag 26 for the particulate inspection system 3500. In alternative embodiments, the agitation sequence may occur at a different time point (e.g. subsequent) mixing. In such embodiments, the agitation sequence may include serial rotations between pairs of angular position. The rotation rate commanded by the control system 15 to the rotary actuator 3470 may be lower than the rotation rate used during mixing. The rotation rate used for agitation may be a function of the frame capture rate of the imagers 3518.

As best shown in FIGS. 178A-D, the agitation sequence may cause particulate within the bag 26 to displace. As the cradle 3311 includes a pattern of repeating dark and light regions 3514A, B, a particle may displace across contrasting regions as a result of the agitation. Thus, particles which may be difficult to visualize on one background color may transit over a second background color against which they may be more easily seen. Providing contrast by temporally adjusting an illuminator 3512 may similarly facilitate visualization of particles which are difficult to see against certain background colors. FIGS. 178A-D depict a black particle 3556B for sake of example. The black particle 3556B is not visible in FIG. 178A as it is in alignment with a dark region 3514B of the background pattern. Due to the agitation of the fluid within the bag 26, the black particle 3556B transits over light regions 3514A of the background pattern as time progresses. As an imager 3518 captures additional frames with the black particle 3556B over light regions of the background pattern, the black particle 3556B is readily distinguishable. This is best shown in FIGS. 178B-D. The size of the light and dark regions 3514A, B may typically be made relatively small to assist in maximizing the number of transitions across light and dark regions 3514A, B for a given particle. A white or light colored particle would complimentarily be readily distinguishable as it moves over dark regions 3514B of the background pattern.

With respect to substantially transparent particles, movement of the particle may similar assist in visualizing the particle within the reservoir or bag 26. The transparent particle will distort light passing through it. The reflected or refracted light would be able to be visualized against the background pattern as the particle displaces. Transparent particles will also distort the background pattern. Again, this may assist in allowing visualization of such particles.

Referring now to FIG. 179, an example data flow diagram 3570 of a particulate inspection system 3500 is depicted. As shown, a number of imagers 3518 are included and each have a field of view 3572 which includes a portion of a bag 26 in place at a cradle 3311. Each of the fields of view 3572 overlaps with that of at least one other imager 3518. The imagers 3518 may capture raw video streams 3574A, B, C. The raw video streams 3574A, B, C may be committed to a log file 3590 associated with the subject bag 26 via a unique identifier for the bag 26. The log file 3590 may also include other identifying information. For example, the log file 3590 may include a date and timestamp. Other potential information may include a unique identifier specific to the individual system 10 in which the particulate detection system 3500 is included or for the particulate detection system 3500 itself. The type of solution in the bag 26 may be included in some embodiments. Software version for the system 10, particulate detection system 3500, and/or neural network version(s) (further described elsewhere herein), any calibration data or settings, user ID information, etc. may also be included in a log file 3590.

In the example, each of the raw video streams 3574A, B, C is also communicated to a server 3576. In other examples, the server 3576 may be replaced with a microprocessor, graphical processing unit, application specific integrated circuit, programmable logic controller, complex programmable logic device, field programmable gate array, or other dedicated processing hardware, and combinations thereof. Dedicated hardware such as an FPGA may be desirable as it may speed processing and remove burden from other processors in the control system 15. Communication may be via a wireless protocol or communication may be wired (e.g. via USB 3.2) depending on the embodiment. The server 3576 may, in some examples, be remote (e.g. cloud) and may receive data from a plurality of client systems 10. Distributed computing environments may also be used.

The server 3576 may feed the raw video stream 3574A, B, C from each imager 3518 into a respective queue. Frames may be extracted from the respective queues and fed into a respective thread. Frames from each thread may then be analyzed for the presence of particles. Analysis on all frames corresponding to a subject bag 26 may be required to be completed before data from another bag 26 is analyzed. In some embodiments, frames of a given bag 26 from each imager 3518 may be processed in series. In other examples, threads of frames from each imager 3518 for a given bag 26 may be processed concurrently.

Analysis of the video may include pre-processing 3578 and detections and tracking processing 3580 (see, e.g., FIGS. 180-182, 192). The pre-processing 3578 may include background subtraction, foreground isolation, filtering of a foreground mask, kernel morphological transformations such as dilations, erosions, openings, closings, morphological gradients, image noise reduction filtering, etc. The pre-processing 3578 may generate processed video streams 3582A, B, C from the raw video streams 3574A, B, C. The processed video streams 3582A, B, C may also be committed to the log file 3590. The pre-processing may also include an analysis of the image data to identify features captured in the frames which are to be determined invalid candidates for further analysis. For example, the pre-processing may identify features which are defined by more than a predetermined number of constituent pixels and flag them as invalid.

The detection and tracking processing 3580 may utilize the processed video streams 3582A, B, C and generate a contents analysis file 3584 (e.g. CSV). The contents analysis file 3584 may specify displacement paths or tracks for any regions of interest within frames that are identified in the processed video streams 3582A, B, C. Image regions of interest may be pixels or pixel collections that appear to displace across the background from frame to frame. Tracks may be determined by comparing adjacent or temporally proximate frames for a pixel cluster that has moved between the frames. If, for instance, the temporally proximate frame includes a pixel cluster within a threshold distance from a pixel cluster's location in the earlier frame, it may be identified as a displacing region of interest. A prediction could be made (e.g. via a Kalman filter) for a pixel cluster's position in a temporally proximate frame and if a pixel cluster in the proximate frame sufficiently corresponds with the prediction, a displacing region of interest may be identified or declared. Clusters of pixels which move across the background may be documented in the contents analysis file 3584, though the contents analysis file 3584 may also include data on single pixel size outputs that displace in this manner as well. The displacement path may be a set of coordinates for each frame in the processed video stream 3582A, B, C. Snapshots of the regions of interest in each frame may also be placed in the contents analysis file 3584. The content analysis file 3584 may also be committed to the log file 3590.

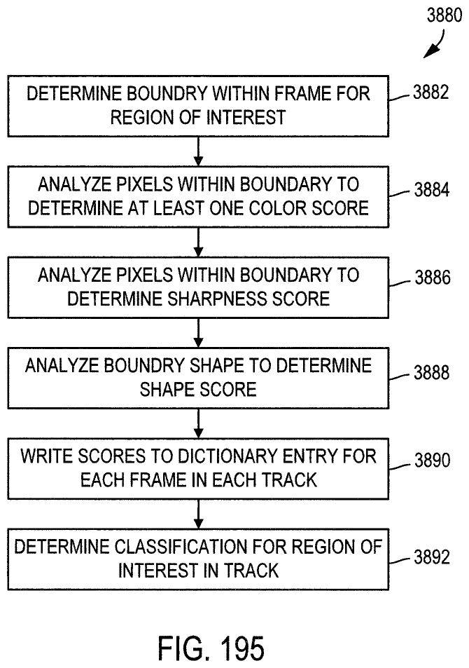

Classifications processing 3586 may be performed to analyze the contents analysis file 3854 to determine if the subject bag 26 includes contents of a first classification, second classification, and potentially further classifications (further described in relation to FIG. 195). The classifications may determine if any of the image regions of interest meet one or more undesirable content identification criteria. Regions of interest may be assigned classifications by the classification processing 3586. Classifications may be binary (e.g. benign or undesirable, particle or bubble, first classification or other, bubble or not bubble) or may include additional information. For example, certain embodiments may classify a region of interest in a genus and potentially a species. For example, the classification processing may identify a region of interest as a particle (genus) of undissolved concentrate (species). Alternatively, the classifications processing could class a region of interest as a particle (genus) and a fiber, hair, or elongate particle (species), or particle of a particular color (species). Various other genus and species type classifications are also possible. The above are merely non-limiting examples. An inspection pass/fail indication 3588 may be generated based on the classifications and output by the classification processing 3586. In the event that no image regions of interest are detected or that none of the image regions of interest meet an undesirable content identification criteria, the classifications processing 3586 may determine the bag 26 is acceptable. In the event that one or more region of interest meets an undesirable content identification criterion, the classifications processing 3586 may determine the bag 26 fails inspection. The classifications processing 3586 may in some embodiments be performed by at least one convolutional neural network which receives as input a cropped image of every region or pixel cluster of interest associated with a given track. Alternatively or additionally, regions of interest may be analyzed by the classifications processing 3586 and assigned various scores (color, blurriness, shape, size, brightness, trajectory, etc.). The scores may be analyzed to generate a classification of the region of interest.

Where a convolutional neural network is used, the convolutional neural network may be trained using bags 26 with known contents. The system 10 used to fill the bags 26 may be reviewed to determine potential sources of particulate. Potential sources of particulate from the bag 26 (or other reservoir) manufacturing process or bag 26 itself may also be identified. Identified potential particulates may then be deliberately introduced into, included in, or created within training bags 26 which are then placed in a particulate inspection system 3500. The training particulate would be representative (size, aspect ratio, density, buoyancy, solubility, color, roughness, material type, etc.) of any particulate encountered during true usage scenarios.

Additionally, the training bags 26 may be generated to entirely or substantially remove confounding contents of interest. For example, in bags 26 dosed with training particulate, the bags 26 may be filled with fluid which has been filtered and degassed. For example, the fluid may be degassed via heating, ultrasonic degassing, vacuum, or some combination thereof. Additionally, for any training bags 26 including particulate, a syringe or similar implement may be introduced into the bag 26 once filled. Remaining air in the bag 26 is withdrawn with the syringe to eliminate or substantially eliminate any headspace in the bag 26. The bag 26 may be utilized for training relatively quickly to prevent any transmission of gas through bag 26 material.

For undissolved concentrate, bags 26 may be dosed with concentrate to their saturation point and additional concentrate may then be added. Alternatively, water vapor may be allowed or encouraged to evaporate through the bag 26 material until, for example, at least some concentrate in a brine contained with the bag 26 falls out of solution. Degassed, filtered water may be added to the bags 26. The bags 26 may be agitated, though not agitated aggressively enough to redissolve the crystallized concentrate and quickly utilized for training to prevent the crystallized concentrate from fully dissolving. Alternatively or for additional training bags 26, a filtered and degassed saturated solution of the same variety as the concentrate already in the bag 26 may be introduced to prevent or mitigate redissolution of the crystallized concentrate.

Training bags 26 containing bubbles but no particulate may also be generated. Two bags 26 may for example be placed into communication with one another. A fluid path from a first to the second bag may be present and may include a check valve upstream of a filter. Fluid from the first bag may be passed through the filter to the second bag. A fluid path from the second bag to the first bag may be present and may include a check valve upstream of a filter. Fluid may be passed from the second bag to the first bag. This may be repeated a number of times. The check valves may ensure flow through each filter is unidirectional. Thus, any particulate in the bags 26 may be collected by the filters and removed from the liquid in the bags 26. Integrity of the filters may be subsequently verified (e.g. with a bubble point test). This may allow a bag 26 which contains air bubbles, but substantially no particulate, to be produced for training.

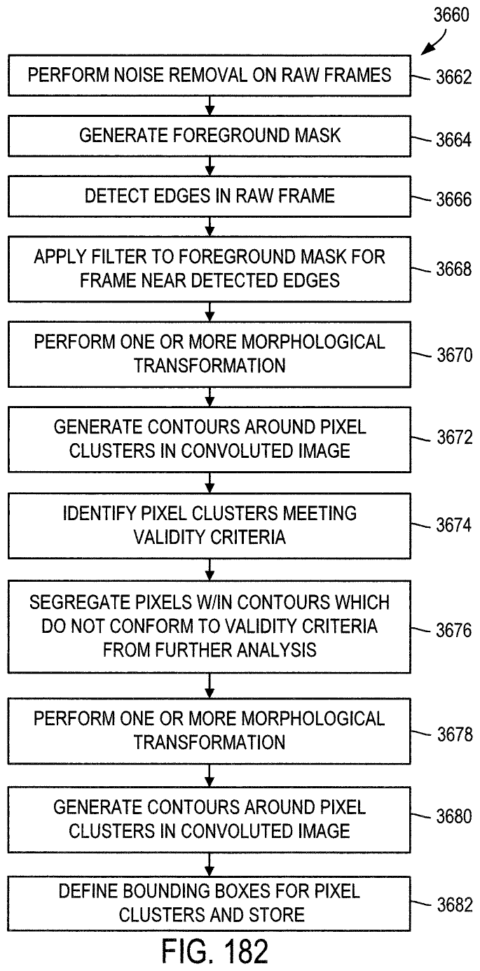

The training bags 26 may be displaced in a mix assisting assembly 2500 according to the displacement profile used during normal operation and images may be captured of the bag 26. Illumination, imager 3518 placement, lens filters, etc. may be kept consistent with true usage conditions. Data from such images may be used as ground truth datasets to train the convolutional neural network. Regions of interest corresponding to the known bag 26 contents may be detected (see, e.g., FIG. 182) and input to the convolutional neural network as training data.

In certain examples, multiple convolutional neural networks may be used. A convolutional neural network for each imager 3518 may, for example, be used. Where individual frames from an imager 3518 are segmented for analysis, each segment may be analyzed by a respective convolutional neural network. This may, for instance, allow for a convolutional neural network to be specialized on images of a specific region of a reservoir. The training data may be sorted by region. Training data from a first region (e.g. the field of view of a first imager 3518) may be used to train a convolution neural network intended to analyze the first region. Training data from a second region (e.g. the field of view of a second imager 3518) may be used to train a convolutional neural network for the second region. Further region specific convolutional neural networks may be trained on training data from the respective regions associated with each of the neural networks.

A pass or fail indication from the classifications processing 3568 may be written to the log file 3590. In the event the bag 26 fails inspection it may be marked as rejected and placed in a quarantine or discard location after removal from the particulate inspection system 3500. The log file 3590 may be communicated to a database 3589 for remote review and analysis.

In some embodiments, a notification may be generated in the event a fail indication is determined. The notification may be generated for display on a graphical user interface 6100 (see, e.g., FIG. 1) of the system 10. Depending on the classification, a suggestion may also be made (and potentially displayed in the notification) as to a likely source (or sources) for the particulate. For instance, a processor of the control system 15 may check the classification against a list of potential sources of particulate. The list could include, for example, score sets for respective potential sources determined using bags deliberately dosed with particulate from those sources (or representative of those sources). Scores closely matching those of an item in the list may cause the suggestion to identify that specific item (or a set of best matches from the list). If the particulate is, for example, classified as green and the only potential green source of particulate is present in the manufacturing environment for the reservoir, the suggestion may indicate the source is likely the manufacturing environment (or a specific component therein).

In the event that excessive computation time has been required for analysis, a notification may similarly be generated for display. For example, if the control system 15 takes greater than a threshold amount of time to detect, track, and classify regions of interest in a bag 26, a fault may be triggered and a notification to that effect may be communicated to a user via a graphical user interface 6100 (see, e.g., FIG. 1). In the event that the number of regions of interest determined to be particles is higher than a threshold, the control system 15 may trigger a fault. A notification may, for example, be generated by the control system 15 indicating an upstream problem in the system 10 or manufacturing environment may be present.

Referring now to FIG. 180, a flowchart 3600 depicting a number of example actions which may be executed to detect the presence of contents of interest within a bag 26 is depicted. A particulate inspection system 3500 may agitate a bag 26 (or other reservoir) in block 3602. This may be accomplished by issuing commands from a control system 15 to a rotary actuator 3470 of a particulate inspection system 3500 (see, e.g., FIG. 158). A dwell time may then elapse in block 3604. The dwell time may vary from embodiment to embodiment, but may be 3-10 seconds (e.g. 5 seconds) in some examples. Once the dwell time has elapsed, the control system 15 may command capture of video frames by each of imager 3518 of the particulate inspection system 3500 in block 3606. When frames are captured, the bag 26 may typically be oriented such that the axes of the ports 392 of the bag 26 extend substantially vertically. In alternative embodiments, the port 392 axes may extend horizontally or at any other angle. At least 70 (e.g. 100) frames from each imager 3518 may be captured over some predetermined period of time for each bag 26. In other examples, less than 70 frames may be collected. Each raw video stream 3574A, B, C may, for example, be captured at a rate of 10 frames per second. Raw video streams 3574A, B, C captured from each imager 3518 for each bag 26 may be ten seconds in length. In block 3608, the raw video streams may be processed by a processor of the control system 15. The processed video frames may be analyzed by a processor of the control system 15 in block 3610.

If, in block 3612, particulate identification criteria are not met, it may be determined that the bag 26 has passed inspection in block 3614. The control system 15 may document the pass and save a log file in block 3616. The control system 15, in block 3618, may command the bag 26 be collected from the particulate inspection system 3500 and displaced to a labeling or marking assembly 3700. The bag 26 may also be labeled with an indication that the bag 26 is acceptable (e.g. embossed with an expiration date) in block 3618. In block 3620, the bag 26 may be displaced to an outfeed from the system 10.

If, in block 3612, particulate identification criteria are met, it may be determined that the bag has failed inspection in block 3622. The control system 15 may document the failure and save a log file in block 3624. The control system 15, in block 3636, may command the bag 26 be collected from the particulate inspection system 3500 and displaced to a labeling or marking assembly 3700. The bag 26 may also be labeled with an indication that the bag 26 is not to be used (e.g. embossed with the word “REJECT”) in block 3626. In block 3628, the bag 26 may be segregated within the system 10. The bag 26 may be placed in a quarantine or discard receptacle, for instance (see, e.g., receptacles 4120B of FIG. 216). Quarantine and labeling may, in some implementations, be accomplished as described in U.S. Pat. No. 11,980,587, issued May 14, 2025, entitled Systems, Methods, and Apparatuses for Producing and Packaging Fluids (Attorney Docket No. 00101.00325.AA697).

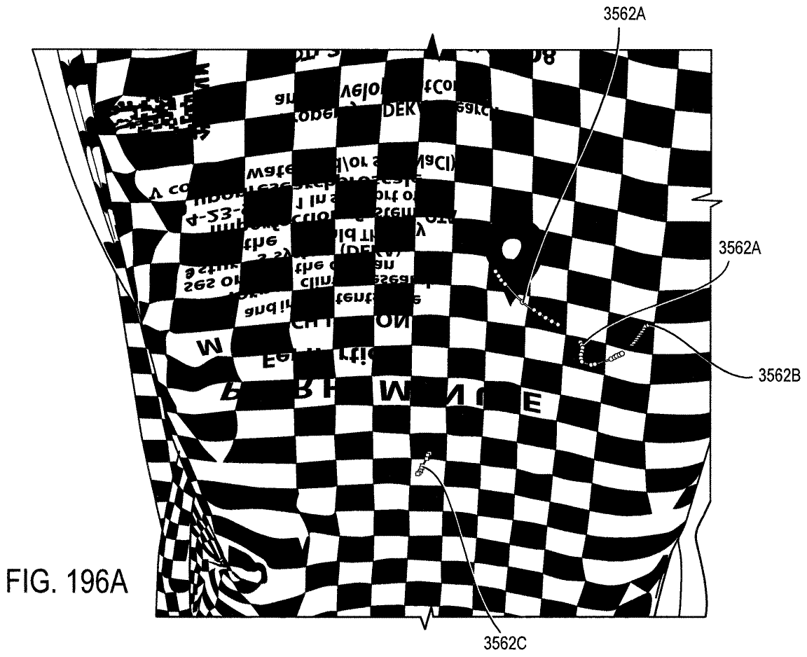

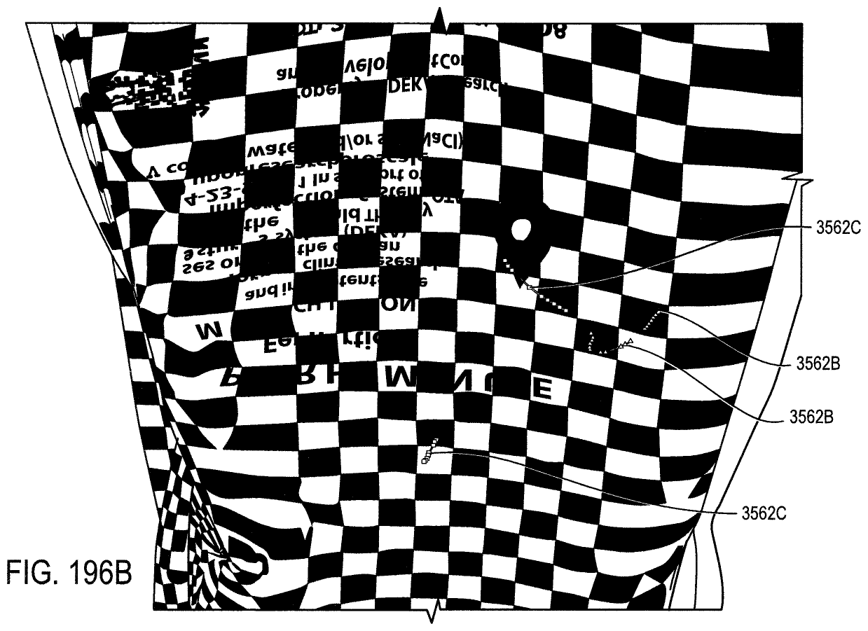

The log files 3590 saved in blocks 3616, 3624 may include any of the raw video, processed video(s), frames thereof, an analysis summary, pass/fail determination, bag 26 or lot identifying information, information about the contents of the bag 26, a unique identifier for the bag 26, etc. In some embodiments, the processed video streams 3582A, B, C may be scaled down before addition to the log file 3590. In other embodiments, a trail may be generated for each region of interest identified and may grow over each frame to show the historical position of the region of interest over the course of the video. The trail may be generated by interpolating between the coordinates of each specific region of interest over adjacent frames. In other examples, a summary may be generated for inclusion in the log 3590. In some examples, the summary may be an annotated version of a frame (e.g. the final captured frame) from each imager 3518. The annotated frame (see, e.g., FIG. 196B) may include a trail of any contents of interest identified in the video frames and a classification of the content of interest (e.g. bubble or particle, bubble/not bubble). Any inspection log files 3590 may be communicated to a database for storage and remote review. Summaries may be or include a data file (e.g. CSV) giving coordinates for each tracked content of interest and respective classification determinations.

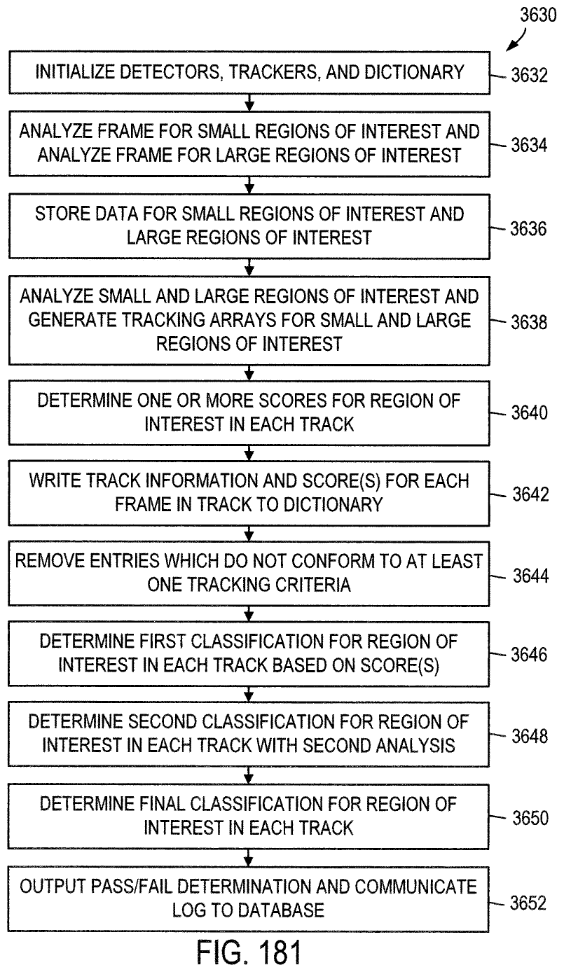

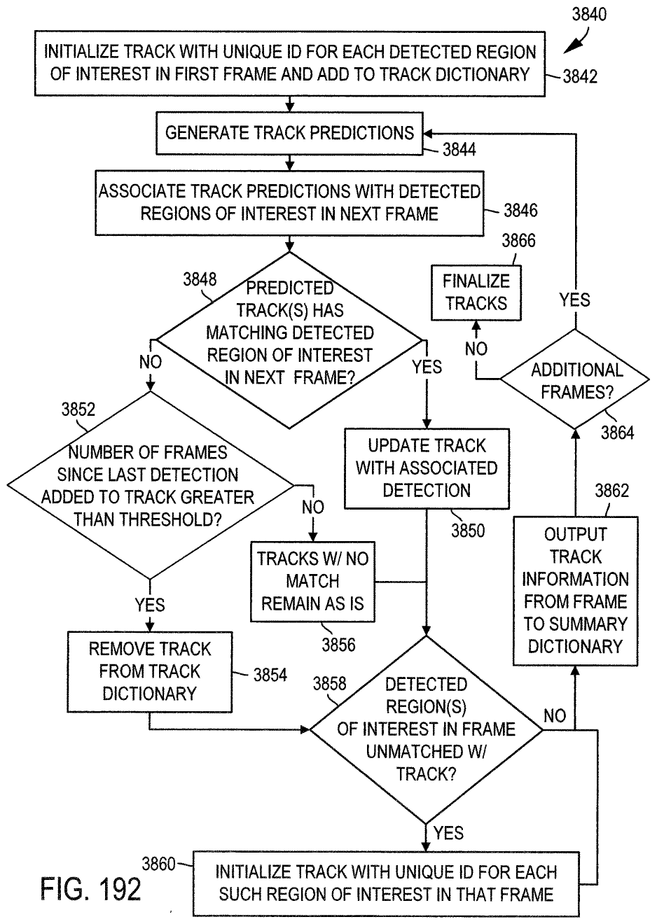

Referring now to FIG. 181, another flowchart 3630 detailing a number of exemplary actions which may be executed to detect and track contents of interest within a bag 26 over a number of video frames is depicted. In block 3632, a processor of the control system 15 may initialize a number of detectors (see, e.g., FIG. 182), trackers (see, e.g., FIG. 192), and at least one dictionary. In some embodiments, a detection dictionary (e.g. an array for storing detection information), a tracking dictionary (an array for storing track information during tracking analysis), and a tracking record dictionary may be initialized. The tracking record dictionary may be a data repository including a selection of information relating to tracks for regions of interest in frames captured by imagers 3518 of a particulate inspection system 3500. Coordinates within a frame, various scores such as any of those discussed herein (see, e.g., FIG. 195), and a clipped snapshot of any regions of interest for each frame may for example be stored in the tracking record dictionary.

In block 3634, each frame from each imager 3518 may be analyzed for small regions of interest and large regions of interest. Frames may be analyzed sequentially and may be analyzed one imager 3518 at a time. Small regions of interest may be, for example, those having an area of 5,000 pixels or less and large regions of interest may be those having an area greater than 5,000 pixels. Regions beyond a certain size (e.g. area greater than 20,000 pixels) may be ignored in certain embodiments. Alternatively, each frame may be analyzed for additional region of interest size ranges (e.g. extra-large having an area greater than 20,000 pixels). Detection of regions of interest is further described in relation to FIG. 182.

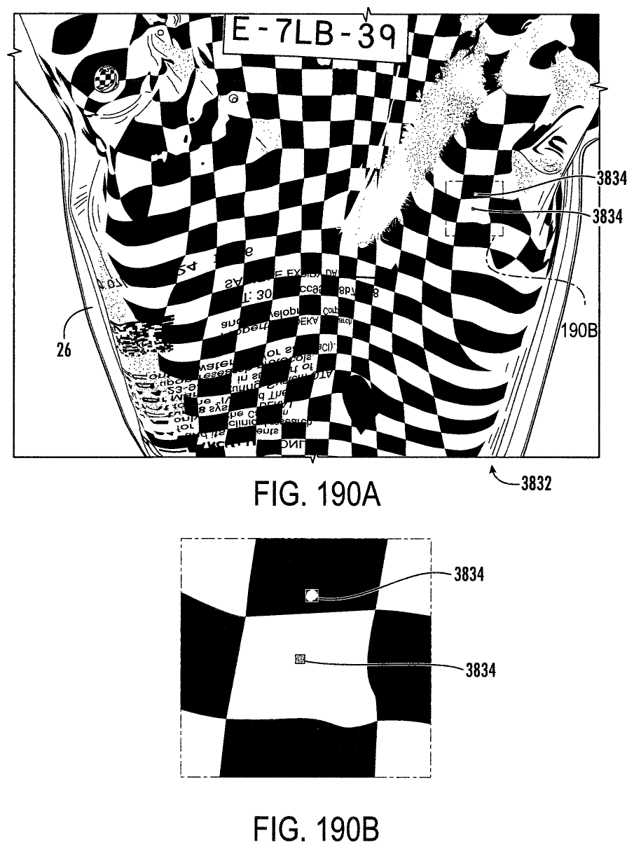

Each identified region of interest for each frame may be assigned a bounding box 3834 (see, e.g. FIG. 190A-B) which delineates the region of interest. The bounding boxes 3834 may be stored (e.g. written to an array) in block 3636. Small and larger regions of interest may be analyzed by a processor of the control system 15 in block 3638 to identify displacement tracks for individual regions of interest across a plurality of frames. Tracking of regions of interest is further described in relation to FIG. 192. Any suitable tracking algorithm may be utilized. For example, a multiple object tracking algorithm may be utilized. In some implementations a SORT (Simple Object Realtime Tracking) algorithm, MHT (multiple hypothesis Tracking) algorithm, JPDA (Joint Probabilistic Data Association) algorithm, etc. may be used.

In block 3640, a processor of the control system 15 may analyze the tracks and assign one or more scores to each identified track. Scoring is further described in relation to FIG. 195. The track information and associated scores may be saved (e.g. written to a tracking record dictionary) in block 3642.

In some embodiments, and as shown in block 3644, entries which do not conform to one or more tracking criteria may be removed. A track may be required to persist over at least some predefined number of frames. A region of interest generating the track may also or instead be required to displace at least some predefined distance (e.g. number of pixels). Tracks which do not meet these criteria may be removed from further consideration. They may be deleted from a tracking record dictionary or separate dictionary used to facilitate region of interest tracking in certain example embodiments.

A first classification for the region of interest associated with each track may be determined based on the at least one score in block 3646. At least one additional classification for the region of interest associated with each track may be determined in block 3648. Each additional classification may be determined with a different variety of analysis. In certain embodiments, a convolutional neural network trained on images of bags 26 with known contents may be used. In block 3650, a final classification may be determined for the region of interest associated with each track. The final classification may be determined based on at least one of the first classification and additional classifications. Alternatively, data (e.g. scores) used to determine any of the non-final classifications may be analyzed to arrive at the final classification. Certain data from one analysis may be weighted heavily or be controlling on the classification determination. For example, in the event a shape score indicates the region of interest is highly elongate, the region of interest has a very low likelihood of being representative of a bubble. The shape score may become controlling or strongly predominate toward as identifying the region of interest as a non-bubble when it is indicative of an aspect ratio beyond a predetermined threshold.

An acceptability determination (e.g. pass/fail) may be made based on the final classification in block 3652. A log 3590 may also be generated in block 3652 and communicated to a database 3502 (see, e.g., FIG. 179) or other electronic storage medium. Electronic memories described herein may be any of a variety of types including optical memories (Blu-Ray, DVD, CD, etc.), USB sticks, flash drives, solid state storage devices, hard drives or other magnetic storage media, secure digital or SD card, database, EEPROM, etc.

The log 3590 (see, e.g., FIG. 179) may include a CSV of track identities and frame by frame coordinates for each region of interest. The log 3590 may also include raw images generated by the imager 3518. At least one set of processed frames (frames after background subtraction, kernel convolution, etc.) generated from the raw images may be included in a log 3590. The log 3590 may also include an annotated version of the final frame collected by each imager 3518 with the tracks overlaid thereon. Lines interpolating the travel path between frame by frame coordinates for each region of interest may be drawn over the image. The final classification may also be indicated on the annotated image. In some embodiments, the frames from each imager 3518 may be stitched together to form a single image of the entire reservoir. An annotated image (see, e.g., FIG. 196B) may be included in the log 3590.

Referring now to FIG. 182, a flowchart 3660 depicting a number of example actions which may be executed to detect regions of interest within images of a reservoir is depicted. Throughout the description of FIG. 182, reference is made to FIGS. 183-191B which provide representations of exemplary images relating to aspects of the flowchart 3660. The representations are not exact reproductions of actual images and are provided for illustrative purposes. Certain features may be removed and others adjusted or exaggerated to facilitate illustration and reproducibility. Where image is used herein to describe the content of a particular drawing, it should be understood the “images” are representative illustrations.

As shown, in block 3662, a processor of the control system 15 may receive each raw frame 3684 and denoise the frames. A representation of a raw frame 3684 is shown in FIG. 183. To denoise the image, values assigned to pixels may, for instance, be adjusted based on an average or weighted average of surrounding pixels. Some specific examples implement Gaussian smoothing for denoising raw frames 3648

The frames from each imager 3518 may be analyzed by a processor of the control system 15 to generate a foreground mask in block 3664. This may be accomplished in any suitable manner. In various examples, a Gaussian mixture based background/foreground segmentation algorithm may be used. An Open CV MOG or MOG2 algorithm may be utilized for instance. In other embodiments, statistical background image estimation combined with per pixel Bayesian segmentation may be used. Deep neural networks, robust principal component analysis (RPCA) segmentation, Dynamic RPCA, etc. are non-limiting alternative options.

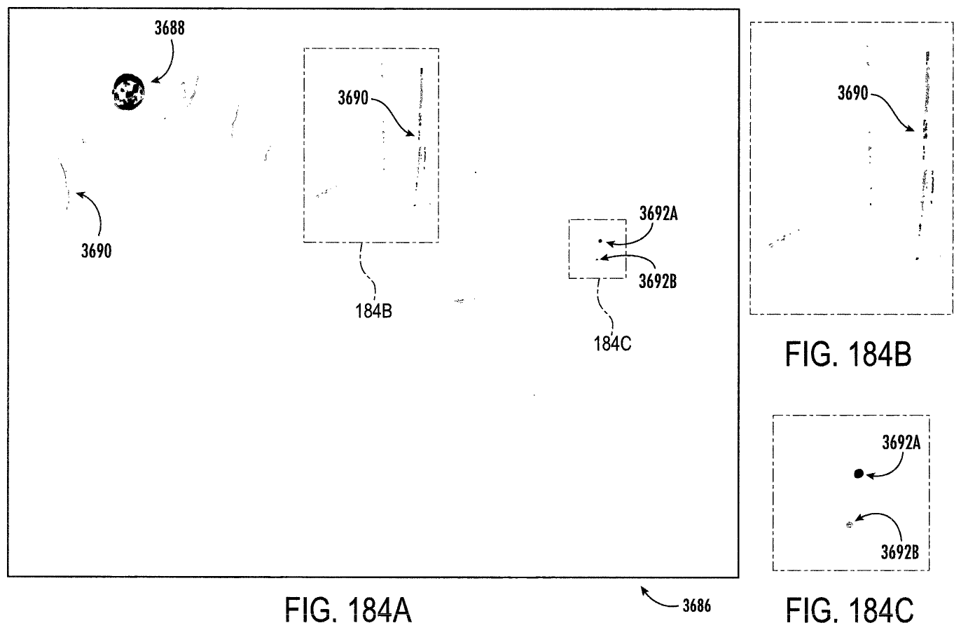

Referring now also to FIGS. 184A-C, a foreground segmented image 3686 representative of what would be generated from the raw frame 3684 depicted in FIG. 183 is depicted. As shown, the dark and light regions 3514A, B of the background pattern on the rest panel 3510 have been removed in the foreground segmented image 3686. Additionally, various edges, crinkles, and folds in the bag 26 or other stationary features are removed. Text on the bag, scratches, logos, appliqués, etc. would be removed. The representative foreground segmented image 3686 provided in FIG. 184A includes a first collection of pixels 3688 in a region corresponding to the location of a relatively large bubble 3838 in FIG. 183. The bubble 3838 is against the wall of the bag and the collection of pixels 3688 may be present due to slight movement of this bubble 3838 and/or settling of the bag 26 over time. As best shown in FIG. 184B, an enlarged portion of the indicated region in FIG. 184A, there may be some small remnants 3690 of the background in the foreground segmented image 3686. These remnants 3690 could be representative of features of the bag 26 or the pattern on the rest body 3510 for instance. The background remnants 3690 may be present, due to subtle settling or shifting movement(s) of the bag 26 over the progression of raw frames 3684 taken by an imager 3518. The representational background segmented image 3686 also includes some putative regions of interest 3692A, B as shown in FIG. 184C (an enlarged portion of the indicated region of FIG. 183A).

Again referring to FIG. 182, in block 3666, a processor of the control system 15 may analyze the raw frames 3684 and detect edges within these frames 3684. An edge detection algorithm may be used for this purpose. Any suitable algorithm may be used. In some examples, a multi-stage algorithm such as a Canny edge detection based algorithm may be used. Gradient based (e.g. Sobel, Scharr, Prewitt, Roberts Cross edge detection) or second order derivative type (e.g. Laplacian) edge detection algorithms may be used. An edge image may be created from each of the raw frames 3684. Binary thresholding may be applied to ensure the edge image is a black and white only output.

A filter may be applied to the foreground segmented image 3686 created from each raw frame 3684 near the detected edges in the respective raw frame 3684 in block 3668. Applying a filter to the foreground segmented image 3686 may assist in filtering out edges detected due to settling motion of flexible reservoirs (e.g. bags 26) over the imaging window. In some embodiments, a morphological transformation may be applied to the edge image. For example, a dilation type kernel convolution may be applied. This may thicken the detected edges to better accommodate shifting of certain types of reservoirs over time. The convolution used may depend on the size of region of interest the detection analysis is tuned for. In some examples, a smaller dilation may be used when detecting large regions of interest. In some examples, no morphological transformation may be used for detecting regions of interest in certain size ranges (e.g. large regions and extra-large regions).

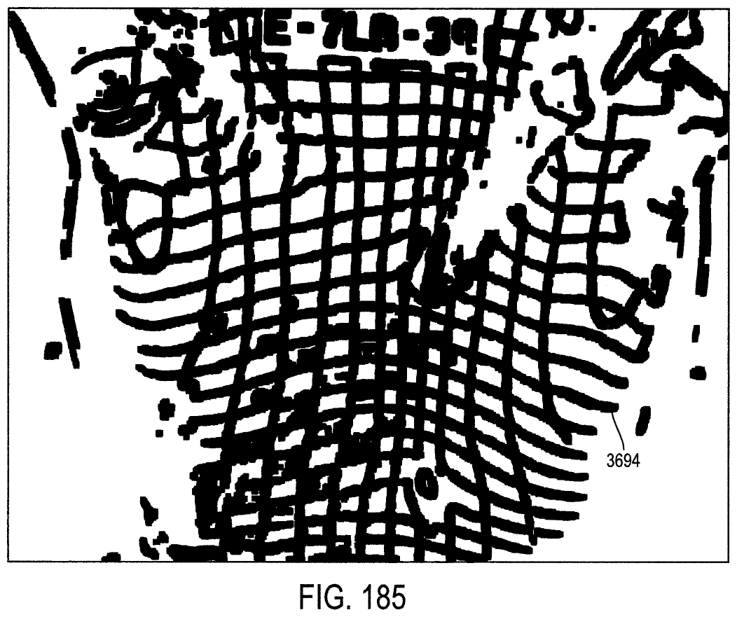

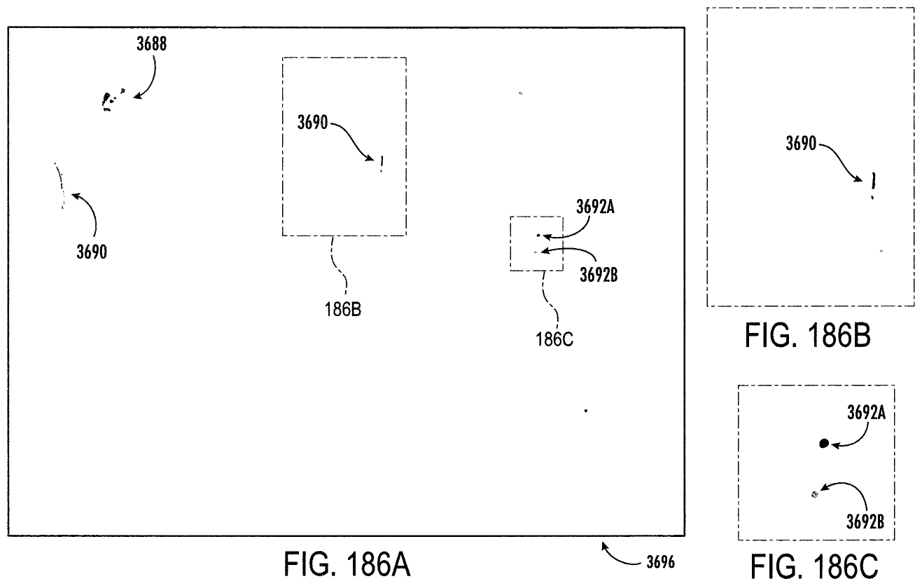

FIG. 185 depicts a visualization of detected edge regions 3694 (after dilation) in the raw frame 3684 represented by FIG. 183. Regions of the raw frame 3684 where edges are determined to be present are shown in black in FIG. 185. A representative near edge filtered foreground segmented image 3696 is depicted in FIG. 186A. The near edge filtered foreground segmented image 3696 is exemplary of what would be generated from the foreground segmented image 3686 of FIG. 184A. As shown best in FIG. 186B the background remnants 3690 may be entirely removed or substantially diminished after applying filtering in regions of the image near detected edges. The example putative regions of interest 3692A, B may be relatively unaltered. Such near edge filtering may not be utilized for detections of certain size regions of interest (e.g. large/extra large).

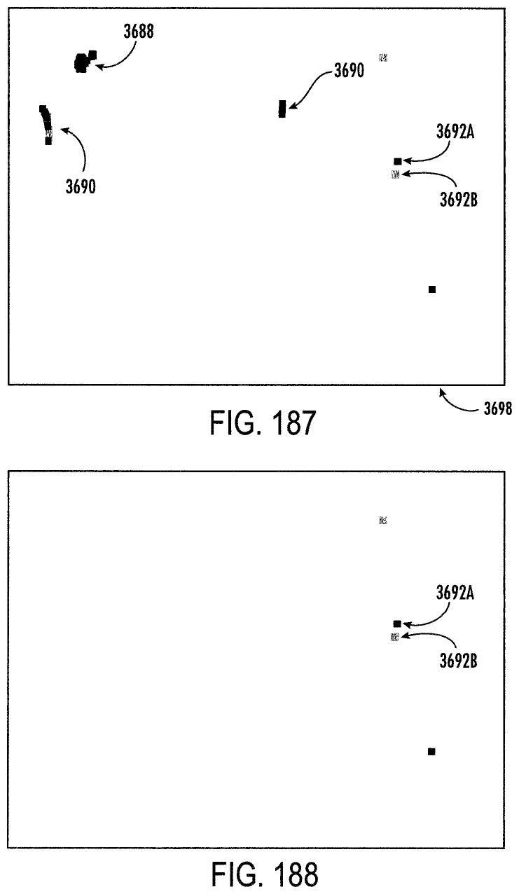

Referring again to FIG. 182, in block 3670 one or more morphological transformation is performed on the near edge filtered segmented foreground image 3696 corresponding to each raw frame 3684 by a processor of the control system 15. In certain implementations a kernel convolution such as a dilation may be performed. A representation of an exemplary dilated image 3698 generated from the near edge filtered foreground segmented image 3696 of FIG. 186A is depicted in FIG. 187. A dilation may make collections of pixels ultimately corresponding to large features in the raw image 3684 become one or more amalgamated regions of pixels. As shown, the collection of pixels 3688 corresponding to the large bubble 3838 in the raw frame 3684 (see FIG. 183) is represented by a contiguous region of black pixels in the dilated image 3698. The background remnants 3690 are also dilated into continuous streaks of pixels. The regions of pixels defining the putative regions of interest 3692A, B are enlarged, but no so much as to merge.

With reference to FIG. 182, contours may be generated around pixel clusters of interest in the convoluted image in block 3672. Contours may be generated via a contour tracing algorithm such as a pixel or border following algorithm, a vertex following algorithm, or a run-data based algorithm. In some examples, a square tracing algorithm, Moore-Neighbor Tracing (e.g. with a Jacob's stopping criterion) algorithm, radial sweeping algorithm, Pavlidis algorithm may be used. The contours may be compared against one or more validity criteria in block 3674. The validity criteria may be a range for the total number of pixels, or area in pixels within the identified contour. If more than a threshold number of pixels are included within the bounds of a contour, the contour may be determined to be invalid. Alternatively or additionally, if less than a threshold number of pixels are included within the bounds of a contour, the contour may be determined to be invalid. This may for example be true when detecting larger regions of interest. Pixels in the dilated image 3698 corresponding to large features in the raw image 3684 and background remnants 3690 may typically be characterized as invalid due to their merger and resultant size at least when detecting small regions of interest. Pixels within contours which do not conform to the validity criteria may be removed from further analysis in block 3676. A representation of the dilated image 3698 of FIG. 187 with pixel clusters in invalid contours (those larger than some preset threshold in the example) removed is shown in FIG. 188.

With reference to FIGS. 187-188, in some examples, a statistical analysis for each pixel cluster of interest may be determined by the control system 15. A confidence interval may, for example, be calculated for each pixel cluster of interest which is representative of the strength of the signal for that pixel cluster. Clusters with a lower signal to noise ratio (e.g. after Gaussian smoothing) would have a wider confidence interval. For sake of example, black is used to depict clusters of interest with tight confidence intervals and gray is used to depict clusters of interest with confidence intervals wider than some predefined threshold. In some examples, if a confidence interval calculation is wider than some predefined threshold, the pixel cluster of interest may be flagged as invalid and removed from further analysis. Alternatively, a confidence interval may be determined for each non-background pixel in an image (see, e.g., FIG. 184A) and carried through the rest of the detection analysis.

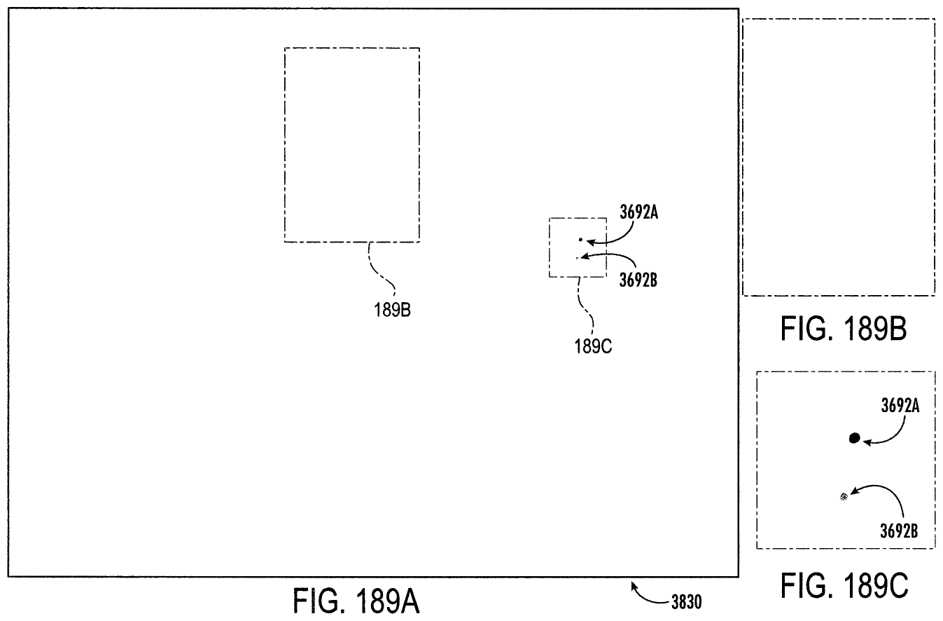

In block 3678, one or more morphological transformation different from that in block 3670 may be performed on the near edge filtered foreground segmented image 3696 created from each raw frame 3684. A kernel convolution such as an erosion may be used. A representation of an exemplary eroded image 3830 generated from the near edge filtered foreground segmented image 3696 of FIG. 186A is depicted in FIG. 189A. The erosion may return or substantially return pixel clusters to their original size and shape. At the same time, the erosion may also cause the pixel clusters have a sharper appearance. As shown, the eroded image 3830 includes the putative regions of interest 3692A, B (best shown in FIG. 189C) but the collection of pixels 3688 is absent. Additionally, as illustrated by FIG. 189B, the background remnants 3690 are absent as well.

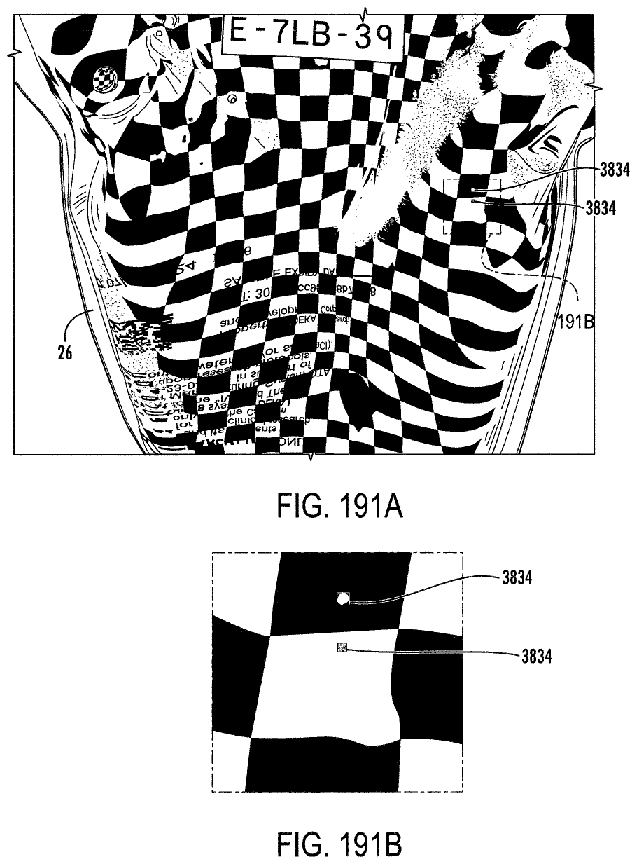

Contours may be generated around pixel clusters of interest in the convoluted image in block 3680. Though reference is made to pixel clusters, it should be understood that a single pixel could also provoke generation of a contour and a single pixel should be understood to be encompassed by the term pixel cluster. Bounding boxes 3834 for each pixel cluster of interest may be generated and stored (e.g. in an array) in block 3682. Bounding boxes 3834 may be defined based on a pixel at a corner of the bounding box 3834 along with a height and width in pixels for that bounding box 3834. A representative annotated image 3832 with bounding boxes 3834 superimposed thereon is depicted in FIG. 190A. An enlarged view of the portion of the image 3832 including bounding boxes 3834 is shown in FIG. 190B. Once pixel clusters are given a bounding box 3834 they transition from putative regions of interest 3692A, B to detected regions of interest. The detected regions of interest may be analyzed to determine respective tracks and classifications if warranted (e.g. are associated with tracks which satisfy validity criteria). As shown in FIGS. 191A-B, the bounding boxes 3834 associated with regions of interest may displace frame to frame. The annotated image 3836 in FIGS. 191A-B is generated from an analysis of a raw frame 3684 taken a number of frames after that shown in FIG. 183. The detected regions of interest may be used in multiple classifications. They may be scored (see, e.g., FIG. 195) and/or may be input to a neural net trained to classify regions of interest in specific embodiments.

Depending on the embodiment, each frame may be processed and analyzed to detect regions of interest a plurality of times. A first time may detect regions of interest which are within a first size range. A second time may detect regions of interest which are in a second size range different from the first (and so on). The pixel count within the area representing the region of interest may define the size of the region of interest. Different tuning may be used for processing and analysis of each frame depending on the size range of regions of interest to be identified. For example, the manner in which the foreground mask is generated or filtered may differ. Kernel convolutions may be more or less aggressive depending on the size range and/or the kernel convolutions may be bigger or small depending on the size range. For the first size range, the size of the kernel may be 20×20 pixels for example. For the second, larger size range, the size of the kernel may be 40×40 pixels. The validity criteria for the analysis of contours in block 3672 may also vary depending on the size range of regions of interest to be identified. The invalid contour criteria (see block 3674) may be arranged to ensure gross motion (e.g. of the bag 26 or large bubbles) is filtered out and not considered for later analysis. That is, regions of interest having a size beyond some predefined threshold may form invalid contours regardless of the size region of interest a particular detection analysis is tuned for. Regions of interest in the first size range may be deemed invalid when contours are generated and analyzed for detections of regions of interest in the second size range.

Additionally, depending on the embodiment, frames from each imager 3518 of a particulate inspection system 3500 may be analyzed differently. Different tuning may be used for processing and analysis of each frame depending on which imager 3518 captured a given frame. This may allow, for example, the bottom of a reservoir, top of a reservoir, and one or more intermediate segments of a reservoir to be treated differently in a content of interest detection analysis. Specific processing and tuning for each size range may be defined for each imager 3518. Likewise, frames collected from a certain imager 3518 may be segmented (e.g. bottom of reservoir, region directly above bottom of reservoir, etc.). Each segment of the frame may be processed and analyzed with its own respective tunings in certain examples.

Referring now to FIG. 192, a flowchart 3840 is depicted detailing a number of example actions which may be executed to track regions of interest within images of a reservoir from an imager 3518. In some embodiments, as with detections, separate analyses may be conducted for regions of interest falling within each respective size range. The same actions may be executed, but would be differently tuned to cater to the respective size ranges for the detected regions of interest. Likewise, separate and different analyses may be conducted depending on which imager 3518 a frame was collected from. Separate distinct analyses may also be conducted for specific regions of a frame from each imager 3518 where frames are segmented.

As shown, in block 3842, a track may be initialized and assigned a unique identifier for each detected region of interest in a first frame. A coordinate and velocity may be determined for each region of interest and associated with the respective track identity. The information may be stored in a tracking dictionary. This information may also be passed to a separate tracking record dictionary in block 3842. In block 3844, the position of the region of interest in the next frame may be predicted for each track. A Bayesian filter such as a Kalman filter may be utilized in certain examples. The track predictions may be associated with matching detected regions of interest identified in the next frame in block 3846. An assignment algorithm may be utilized for this purpose. For example, a bipartite matching algorithm or specifically the Hungarian Method algorithm may be utilized. The total cost used for such an assignment algorithm may be measured in, but is not limited to being measured in, distance (e.g. in pixels), intersection over union, mean square error, or some combination thereof.

If, in block 3848, a predicted track has a matching detection in the following frame, the respective track may be updated with the associated detection in block 3850. The coordinates for the region of interest and the velocity of the region of interest for the frame may be added to the track. If a predicted track has no matching detection in block 3848, a check may be made as to the number of frames since a detection matching that track has been found.

In the event that the number of frames since the last detection assigned to a track is greater than a predefined threshold in block 3852, the track may be removed from the tracking dictionary in block 3854. This may decrease resource demand as predictions for this track would no longer be determined. It may also increase the rapidity with which frames may be analyzed for tracking purposes. Where the particulate inspection system 3500 is included in a fluid production and packaging system 10, this may facilitate an increase in system 10 throughput. If, in block 3852, the number of frames is less than the threshold, the track may be retained in the tracking dictionary, but not updated in block 3856.

If, in block 3858, there are detected regions of interest in the current frame which have not been matched with a track, respective new tracks with unique identities may be initialized in block 3860. In the event a frame includes a detection for a region of interest which generated a previously removed track (see block 3854), a new track would be initialized and the region of interest would be tracked under a different unique identity. Optionally, a processor of the control system 15 may assign removed tracks to tracks initialized in later frames. Thus separate tracks may be assembled into reconnected tracks under a single identity in certain embodiments. For example, if a detection several frames after a track has been terminated is within a predicted position range of the end of the terminated track, the terminated track may be connected to the track associated with that subsequent detection. In some examples, this may be done only if there are no other detections initializing new tracks in the predicted position range from the terminated track for a preset number of frames.

After initializing any new tracks in block 3860, or if no detections requiring new track initializations are present in block 3858, the track information for the frame may be output to the tracking record dictionary in block 3862. Thus, though tracks may be removed from the tracking dictionary, the tracking record dictionary would contain a record of all trackings with unique identifiers and frame by frame detection information for each track.

If, in block 3864, there are no additional frames, the tracks may be finalized in block 3866. If, in block 3864, additional frames are present, the flowchart 3840 returns to block 3844.

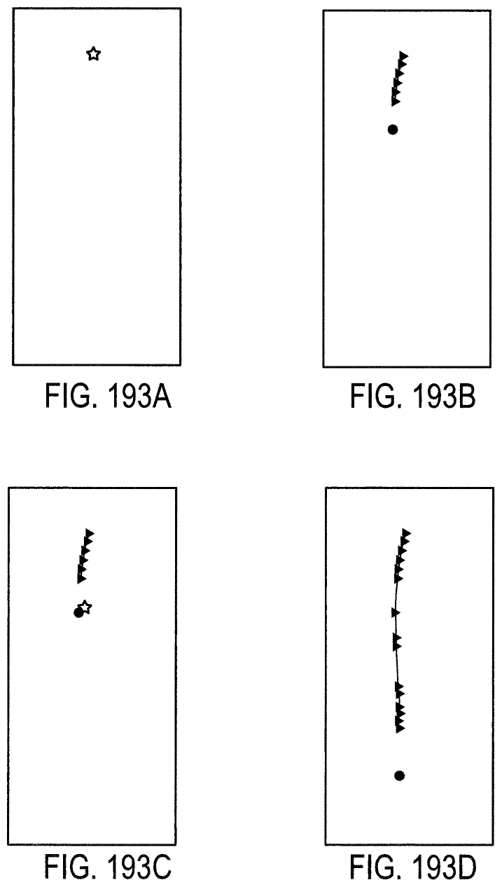

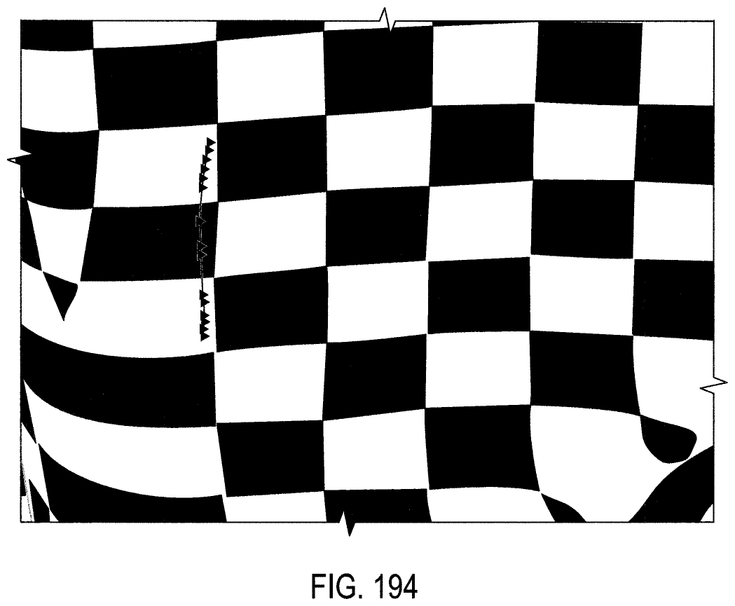

With reference now also to FIGS. 193A-194, a series of example visualizations illustrating tracking of an example region of interest are depicted. FIGS. 193A-D depict representations of various frames after detections processing (see, e.g., FIG. 181) has been completed. The detected region of interest in each frame (if present) is represented with a star. Past locations for the detected region of interest are superimposed on the frames (triangles) along with location predictions (circular dots) for the region of interest in each frame. FIG. 194 depicts the example track shown throughout FIGS. 193A-D overlaid onto a representation of a portion of an image of a bag 26.

As shown in FIG. 193A, a detected region of interest may be present in a first frame. This may result in initialization of a track (see block 3842 of FIG. 192). Track predictions may be generated and associated with detected regions of interest in subsequent frame (see blocks 3884, 3886 of FIG. 192). This may begin to create a track which documents displacement of the region of interest over the series of frames (see, e.g., FIG. 193B).

As indicated in blocks 3848, 3852 and 3856 of FIG. 191, the prediction for a region of interest may not match with a detection in a subsequent frame. In this event, predictions may continue to be generated based on the most recent known information for the region of interest associated with the track. FIG. 193B shows a prediction a number of frames after the last match for a detected region of interest. If a subsequent frame includes a detected region of interest corresponding to the prediction for that frame, the detected region of interest may be assigned to the track. In FIG. 193C, a detection is present in close proximity to the prediction for that frame and would be assigned to the example track. This may, for example, help to prevent a dark region of interest passing over a dark background section from causing a track to terminate. Additionally, it may assist in accommodating near edge filtering of a foreground segmented image. A track may, however, terminate if no detected region of interest matching a track prediction has been found for some threshold number of consecutive frames (see, e.g., block 3852 of FIG. 192). FIG. 193D depicts a track with a prediction having no matching detection the threshold number of frames since the last detected region of interest was added to the example track. As no matching detected region of interest was found in the threshold frame, the example track may be determined to be complete. This completed track is shown overlaid on an illustrative representation of an illustration of a raw image in FIG. 194. As described in FIG. 192, data corresponding to the example track would be memorialized in the tracking record dictionary, but removed from the working tracking dictionary used for generating predictions in the next frame to limit computational burden during processing.

Referring now to FIG. 195, a flowchart 3880 is depicted detailing a number of example actions which may be executed to score regions of interest associated with tracks in images of a reservoir. In the following discussion, the score is generated based off of an analysis of each frame in a given track. In other embodiments, a subset of the frames or a single frame of the track (e.g. final frame) may be utilized. In block 3882, a processor of the control system 15 may define a scoring boundary around the region of interest in each frame from a track. The scoring boundary may be a fixed and preset size. For example, a scoring boundary of not more than 20×20 pixels centered on the region of interest may be used. The size of the scoring boundary may be chosen to ensure an entire region of interest would be captured by the scoring boundary. The scoring boundary size may differ in alternative embodiments. Different size scoring boundaries may also be used depending on the size of the region interest the detection process was tuned for. For example, a larger boundary may be used if the detected region of interest was identified by a detection process tuned for extra-large regions of interest. Different size scoring boundaries could also be used for each respective imager 3518 or for different segments of a frame collected by a single imager 3518.

In block 3884, a processor of the control system 15 may analyze the pixels within the scoring boundary for each frame to determine at least one color space score. In some implementations, the processor may determine a count for the number of pixels which have color space values which fall within predefined color space value ranges (e.g. ranges of RGB values). This count may be assigned as the color space score. As mentioned in relation to FIG. 159, an external illuminator 3516 may be selected to emit light in a color (e.g. red) which is selected to differ from the color of potential sources of particulates. In example embodiments, this helps to highlight bubbles within a bag 26 or other reservoir. The color space value ranges may be specified to correspond to the light color emitted by the external illuminator 3516. Thus, scoring boundaries yielding a high color score in a particular defined value range may be suggestive that the tracked region of interest is a bubble. The predefined color space value ranges may also be selected to correspond to bright regions with a reservoir. Again, due to the optical properties of bubbles, bubbles should appear particularly bright. Regions with a high brightness score may be suggestive that the tracked region of interest is a bubble.

In block 3886, a processor of the control system 15 may analyze the pixels within the scoring boundary to determine a sharpness score. A fast Fourier transform or the variance of the Laplacian of the image within the scoring boundary could be used. Bubbles may typically be relatively sharp due to their interaction with light from the light emitter 3516. A scoring box with a high sharpness score may be indicative that the tracked region of interest is a bubble.

In block 3888, a processor of the control system 15 may determine a shape score for the region of interest over the frames of a track. The shape score may, for example, be determined based on the bounding box generated from the contours of the region of interest when the region of interest was detected (see, e.g., blocks 3680, 3682 of FIG. 182). The shape score may be determined based upon the aspect ratio of the bounding box. As bubbles are substantially spherical, they should have an aspect ratio of 1:1 (or nearly 1:1). More rectangular aspect ratios are suggestive of particulate. The aspect ratio of the shape score for a region of interest may be averaged over all frames in a given track to determine an average shape score. This in turn may be used to determine the shape score. In some examples, additional or alternative statistical processing may be performed. For example, a standard deviation of the aspect ratios for the region of interest across frames in the track may be determined. Standard deviations above a certain threshold may be indicative of rotating particulate. In certain examples, invalid shape scores may be identified and the corresponding regions of interest may be excepted from further analysis. This captures artefacts created during filtering or other image processing allowing them to be removed from the analysis.

In block 3890, scores from each analysis may be stored (e.g. written to a tracking record dictionary). A classification for the tracked region of interest may be determined based on the scores in block 3892. Certain scores may be weighted more heavily than others. For example, if a very high color space score is present, it may heavily weight the determination towards classifying the region of interest as a bubble 3550. If a shape score indicative of a highly elongate region of interest is present, it may heavily weight the determination towards classifying the region of interest as particulate. Weighting of scores may also differ depending on the imager 3518. For example, scores indicative of a bubble may receive heavier weighting in frames from an imager 3518 capturing the highest portion of the reservoir.

In some embodiments, additional scores may be determined. For example, a trajectory score may be determined. The frame to frame position data for a region of interest may be analyzed to determine a direction of motion characterization for the region of interest. Velocity data may further be analyzed. The trajectory score may be an indicator of whether the region of interest appears to be sinking or rising within the bag 26. Bubbles 3550, for example, may be particularly buoyant and may tend to rise while certain types of particulate may tend to sink due to their density. Additionally, trajectory scores may be an indicator of size. For example, a larger particle of dense material would be more likely to sink (or rapidly sink).

Referring now to FIG. 196A, a log file 3590 may be generated after analysis and classification of images. An example visualization 3560 of an analysis of frame captures from an imager 3518 monitoring a bag 26 including particulate is shown in FIG. 196B. The example visualization 3560 is an annotated image. The image selected for annotation may, for example, be a final frame capture of the raw video taken by an imager 3518. In some embodiments, the images from all imagers 3518 may be stitched together (e.g. corresponding frames taken at the same time point) to from the annotated image or individual annotated images for the frame sets output by each respective imager 3518 may be generated. As described in detail above, once a region of interest in a set of frame captures has been detected, its displacement path or track within the bag 26 may be determined. The control system 15 may make a classification determination on the region of interest. As shown, each trace 3562A-C within the visualization 3560 depicts the path of a region of interest within the bag 26. The traces 3562A-C included in the visualization 3560 may be drawn from the coordinates associated with any track identities present in the tracking record dictionary (further described in relation to FIG. 192) for a set of frames. The traces 3562A-C are given different indicia to communicate their classification. Circles, squares, and triangles are used in the example, however, color, text, symbols, or other indicators may instead or additionally be used. Such visualizations 3560 may be generated for each bag 26 and placed in a log 3590 (see, e.g., FIG. 179) associated with each bag 26. A unique identifier for each trace 3562A-C (e.g. the associated track identity in the tracking record dictionary) may be superimposed over the image in the visualization 3560. The unique identifier may also be used in the log file 3590 in association with data related to the region of interest forming the respective trace 3562A-C. The log 3590 may, for instance, include the data or at least a portion of the data from the tracking record dictionary. Where a bag 26 includes no particulate, the visualization 3560 may be devoid of traces 3562A-C. The annotated image may explicitly state no tracks were identified.

Referring now also to FIG. 196B, in certain embodiments, multiple classifications may be made for the region of interest data sets collected for each bag 26. One classification may, for example, be based off classification logic which determines a classification for each region of interest based on score characteristics determined for the region of interest. Another classification may be generated by a trained machine learning model. The trained machine learning model may be a convolutional neural network trained on images of bags 26 with known contents. Though various examples described herein refer to convolutional neural networks as an exemplary trained machine learning model, any suitable alternative trained machine learning model may be used in alternative embodiments. The multiple classifications for each respective region of interest may be analyzed to determine a final classification. This may be a weighted determination with the output of one or more of the classifications being more controlling than others. The annotated image in FIG. 196B may be representative of the final classifications determined for a given bag 26. Thus, each individual classification could be associated with a respective annotated image and an annotated image for the final classification may be all be included in the log 3590.

In some embodiments, a second classification may only be made for regions of interest with unclear classification or low confidence classifications. For example, the traces 3562A classed with a circle in FIG. 196A may represent regions of interest with an unclear classification. The data associated with these regions of interest may be passed to secondary classification processing which may attempt to assign a classification. As shown in FIG. 196B, no traces 3562A classed with a circle are present. The traces 3562A classed with a circle have been replaced by traces 3563B-C classed with squares and triangles. Using the example of an IV bag 26, the squares may represent particulate while the triangles may represent bubbles. Thus, a second of the multiple classifications may be used to clarify any regions of interest with unclear or low confidence classification.

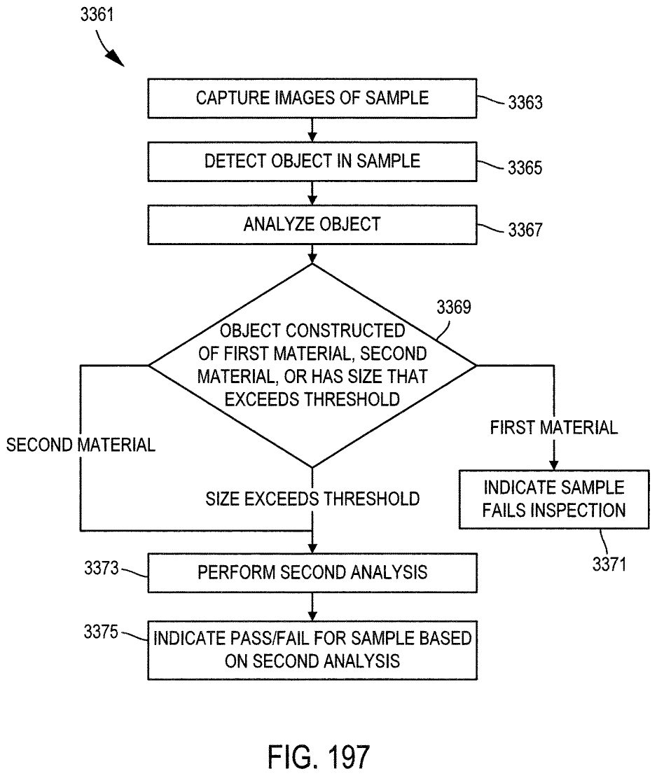

Referring now to FIG. 197, a flowchart 3361 detailing a number of example actions which may be executed to inspect a sample is depicted. Images of the sample (e.g. bag 26) may be captured in block 3363. In block 3365, objects may be detected in the image. In block 3367, detected objects may be analyzed. If, in block 3369, the analysis indicates an object is of a first material, the sample may be indicated to have failed inspection in block 3371. If, in block 3369, the analysis indicates the object is constructed of a second material, a second analysis may be performed in block 3373. Similarly, if, in block 3369, the analysis indicates that the object has a size that exceeds a threshold, a second analysis may be performed in block 3373. The sample may be assigned a pass/fail indication in block 3375 based on the second analysis.

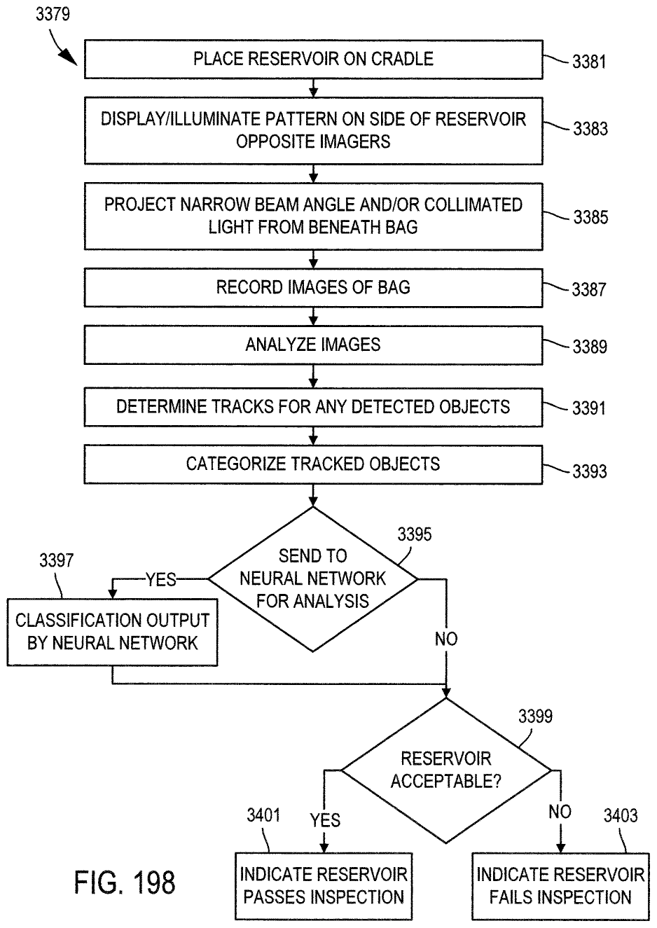

Referring now to FIG. 198, a flowchart 3379 detailing a number of example actions which may be executed to inspect a sample is depicted. In block 3381, a sample reservoir (e.g. bag 26) may be placed on a frame 3309 or cradle 3311. A pattern may be displayed or illuminated behind the sample reservoir in block 3383. In block 3385, narrow beam angle and/or collimated light may be projected from beneath the sample reservoir. Images of the sample reservoir may be recorded in block 3387. Typically, the sample reservoir is agitated or mixed prior to imaging. In block 3389, the images may be analyzed. Tracks for any detected objects may be determined in block 3391. Detecting and tracking may be as described elsewhere herein. Tracked objects may be categorized in block 3393. If, in block 3395, the object has a certain categorization, the object may be classified by a neural network in block 3397. Otherwise, a determination may be made as to whether the object causes the reservoir to fail inspection without classification by the neural network. The categorization or classification from the neural network may be used to determine whether the reservoir passes or fails inspection. If, in block 3399, the reservoir is determined to be acceptable, the reservoir may be indicated as having passed in block 4301. If, in block 3399, the reservoir is determined to be unacceptable, the reservoir may be indicated as having failed inspection in block 4303.

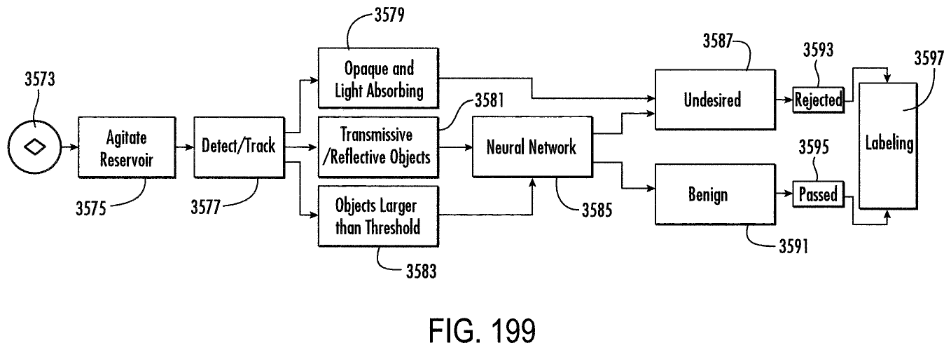

Referring now to FIG. 199, an example data flow diagram 3571 for a particulate inspection system 3500 is depicted. At block 3573, a bag 26 (or alternative reservoir type) may be placed in a particulate inspection system 3500. The bag 26 may be agitated or mixed at block 3575. The bag 26 may then be illuminated by first light source(s) 3521 and a backlight for the background pattern (e.g. second light source 3511). Image data for the bag 26 may be captured by one or more imagers 3518. At block 3737, the captured images may be analyzed to detect contents of interest within the bag 26. Tracking for any detected contents of interest may also be performed. Detection and tracking of contents of interest may be as described elsewhere herein.

Detections (and potentially tracks associated with the detections) may be categorized as at least one of an opaque and light absorbing object 3579, a light transmissive or reflective object 3581, and objects having a size greater than a predefined threshold (e.g. 180 μm) 3583.

An object determined to be opaque and light absorbing with a size greater than the threshold may be classified as an undesirable content. Such objects may include free floating materials having a high opacity (e.g. at least 60% opaque to visible wavelengths) and may create moving shadows detected by background subtraction (and potentially various other processing described herein). Other objects, such as one determined to be transmissive, reflective or a sized in excess of the predefined threshold, may be classified, in some embodiments, via a neural network (block 3585). In certain embodiments, the neural network may be a convolutional neural network or CNN trained on a wide set of data pertaining to microbubble/particulate identification. The neural network may determine which detections/tracks are classified as being benign contents 3591 (e.g. bubbles 3350) and which are undesired contents 3587 (e.g. particulate). The neural net may help to limit false classification of bubbles 3550 as other, undesirable contents which would otherwise cause a bag 26 to be failed and discarded. As shown benign contents 3591 may be logged with a pass in block 3595. Reservoirs having at least one undesired content may be logged with a fail in block 3593. Bags 26 may be labeled accordingly (e.g. via a marking assembly) at block 3597.

Referring now to FIG. 200, a marking assembly 3700 may be included in various systems 10. The marking assembly 3700 may create one or more marking on a bag 26 before a bag 26 is dispensed from the system 10. Though described in relation to a bag 26, it should be understood marking assemblies 3700 described herein may be used for any of a variety of other target marking substrates. For example, marking assemblies 3700 described herein may be particularly well suited for use with flexible packaging, film packaging, blister packaging, etc. Description of the marking assembly 3700 in the context of bags 26 is non-limiting and merely exemplary.

Markings on, for example, bags 26 may be made in any of a variety of different manners. For example, the marking may be embossed to the bag 26, printed or stamped onto the bag 26, created on a label applied to the bag 26, generated via a laser, etc. Preferably, a marking assembly 3700 may mark the bag 26 without a consumable component (e.g. ink, adhesive backed labels, etc.). Additionally, preferred marking assemblies 3700 may not generate or generate minimal vapor, fumes, particulate, or other detritus when placing a marking on a bag 26. In certain examples, the marking assembly 3700 may mark the bag 26 by exerting a mechanical stress upon a portion of the bag 26 which is sufficient to alter an optical property of the bag 26. For example, an optical property of a region of the peripheral seal of the bag 26 may be subjected to mechanical stress sufficient to cause stress whitening of the bag 26 material. The marking assembly 3700 may emboss or peen the bag 26 to generate a marking.

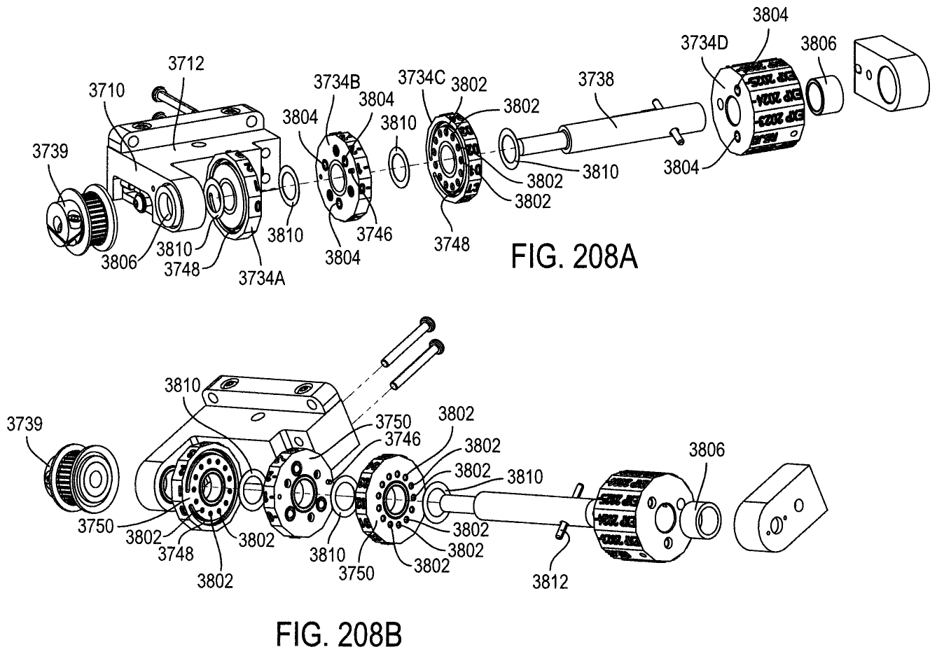

Still referring to FIG. 200, a block diagram of an example marking assembly 3700 with an embossing head 3702 is depicted. The embossing head 3702 may include an exterior surface 3704. The exterior surface 3704 may include a number of marking bodies 3706 raised therefrom. The marking bodies 3706 may include one or more typeset character. For example, the marking bodies 3706 may include one or more alphabetic, numerical, or punctuation character. In alternative embodiments, the marking bodies 3706 may additionally or instead include pictograms, symbols, or any other desired design. The embossing head 3702 may be rotatable such that different marking bodies 3706 may be displaced into a marking position depending on the mark desired to be created with the marking assembly 3700. In the example embodiment, a rotary actuator 3708 is coupled to the embossing head 3702 to rotate the embossing head 3702. Though the example embossing head 3702 is depicted as a round body, other embodiments (see, e.g., FIG. 208A) may include an embossing head 3702 which has a polygonal cross-section. The exterior surface 3704 may thus include a number of faces. Each face may include one or more marking bodies 3706 thereon.

As described in detail below, the embossing head 3702 may be divided into one or more independently displaceable segments. The segments may be individual rotors 3734A-D (see e.g. FIG. 208A) on a shaft 3738 of the embossing head 3702 in some embodiments. Each of the independently displaceable segments may be rotated to place a desired marking body 3706 into a marking position. In such examples, the independently displaceable segments may be displaced in order to create a desired combination of marking bodies 3706 in a marking position to create a specified mark. The mark may be a string of text (e.g. letters, numbers, punctuation, some combination thereof), may be a date, lot code, unique identifier for the bag 26 or system 10, indication of solution type within the bag, or a combination thereof. Preferably, the independently displaceable segments may each be driven through a single rotary actuator 3708.

The embossing head 3702 may be coupled to a trunnion 3710 including a base 3712. The trunnion 3710 may be coupled to an embossing actuator 3714. The embossing actuator 3714 may be any suitable variety of actuator and may in certain implementations include one or more pneumatic cylinder. Preferably, the embossing actuator 3714 may provide 750-1000 flb of force or more. Alternatively, the embossing actuator 3714 may provide up to 490 flbs.

The embossing actuator 3714 may displace the embossing head 3702 and coupled components, via commands from the control system 15, from a retracted position to an embossing position against a bag 26 (or other work piece). The embossing actuator 3714 may hold the embossing head 3702 in an impressing position for a dwell period (e.g. less than a second) and then the control system 15 may command retraction of the embossing head 3702. One or more position sensor 3715 may be included to monitor the position of the embossing head 3702. Though any suitable position sensor variety may be used, in certain examples, magnetic switch type sensors may be utilized. In the example, a first position sensor 3715 and second position sensor 3715 are included. A magnetic body on the cylinder of the pneumatic piston of the embossing actuator 3714 may trip the first position sensor 3715 when the embossing head 3702 is in a retracted state. The piston magnet may trip the second position sensor 3715 when the embossing head 3702 is in an impressing position. The control system 15 may monitor the output signal from the embossing head position sensor 3715 to confirm that the embossing head 3702 has displaced to the impressing position before beginning a timer from the dwell period. The control system 15 may also verify that the embossing head 3702 was successfully retracted via the output signal of the position sensor 3715 before commanding displacement of the bag 26 from the marking assembly 3700. Where the embossing actuator 3714 includes one of more pneumatic cylinder, the control system 15 may confirm the pressure supplies for driving the embossing actuator 3714 are within predetermined respective target ranges before orchestrating actuation of the embossing head 3702.

As shown in FIG. 200, a backstop assembly 3716 is included. The backstop assembly 3716 may include a backstop 3717 disposed opposite the embossing head 3702. The backstop 3717 may be a rigid member which is supported against deflection or other movement against any force exerted by the embossing actuator 3714. The target region of a bag 26 may be displaced into a gap between the backstop assembly 3716 and the embossing head 3702. In some examples, the backstop assembly 3716 may include one or more guide 3718 which may assist in leading the target region of the bag 26 into alignment with the embossing head 3702. The backstop assembly 3716 may include a compliant body 3720 coupled to the backstop 3717 and disposed directly opposite the embossing head 3702. As the embossing actuator 3714 drives the embossing head 3702 against the target region of the bag 26, the marking bodies 3706 in the marking position may press into the complaint body 3720 through the bag 26. The compliant body 3720 preferably may not permanently deform as the marking bodies 3706 press into the compliant body 3720. In some examples, the compliant body 3720 may be formed of a polyurethane material. In such examples, a polyurethane with a shore durometer of 75D may be utilized.

In various examples, the complaint body 3720 may be disposed in a pocket 3722 in the backstop 3717. The width dimension of the pocket 3722 may be less than the width of the embossing head 3702, but greater in width than the greatest distance between any marking bodies 3706 in the marking position. The pocket 3722 may have a depth greater than the distance the marking bodies 3706 are raised from the exterior surface 3704 of the embossing head 3702. In the event the compliant body 3720 degrades, is dislodged, or otherwise compromised, unadorned edge portions of the embossing head 3702 may bottom out on the flanks of the pocket 3722 defined by the backstop 3717. This may establish a mechanical interference which prevents the marking bodies 3706 from being driven into the material at the bottom of the pocket 3722. Thus, damage to the marking bodies 3706 may be avoided. Alternatively or additionally, marking bodies 3706 may be positioned on a face of the exterior surface 3704 of the embossing head 3702 leaving an unadorned margin above and below the marking body 3706 on that face. That is the height of the marking bodies 3706 may be less than the height of the face of the exterior surface 3704 from which they project. The height dimension of the pocket 3722 for the complaint body 3720 may be greater than the height of the marking bodies 3706, but less than the height of the faces of the exterior surface 3704 on which the marking bodies 3706 are included. In the event of a compromised compliant body 3720, the margins above and below a marking body 3706 may bottom out on the material surrounding the pocket 3722. Again, this may inhibit marking bodies 3706 from being driven into the material at the bottom of the pocket 3722 similarly avoiding damage to the marking bodies 3706.

As shown, an imager 3733 may be included as part of the marking assembly 3700. The imager 3733 may capture an image of the marking made on the bag 26. The image may then be analyzed to ensure that the marking is as expected and is sufficiently visible. The control system 15 may, for example, perform an optical character recognition to determine the content of the marking. This determined marking content may be compared to the desired mark to ensure the marking on the bag 26 is acceptable. In the event that the marking is determined to be unacceptable, at least one additional different marking may be made on top of the unacceptable marking to make the initial marking unintelligible. The marking may again be analyzed to ensure that no intelligible marking may be determined via optical character recognition. Additional different markings may be overstamped on the initial marking until recognition analysis fails to detect an intelligible marking.

After marking of a bag 26, various data may be written to the batch record for that bag 26. The control system 15 may for example write one or more of the intended marking for the bag 26 (e.g. character string), the determined marking content from imager 3733 data (e.g. a character string), the raw image from the imager 3733, an annotated version of the raw image 3733 noting the determined marking content, the pressure applied during embossing, and temporal data relating to the marking operation (e.g. start and stop time, dwell time). Preferably, the field of view of the imager 3733 may capture both the marking and the indicia 56 (see, e.g., FIG. 159) on the bag 26. A pass/fail indication for the marking may also be included in the batch record for the bag 26. The control system 15 may also track and update odometry data relating to the marking assembly 3700. Various odometry values may be tracked. For example, the control system 15 may increment a number of bags 26 marked by the marking assembly 3700 each time a bag 26 is marked. Additionally, the number of bags 26 may be tracked and marked for each marking body 3706. In some embodiments, the control system 15 may generated a notification instructing a user to schedule service in the event that one or more odometry value exceeds a threshold.

Referring now to FIG. 201, a perspective view of an example embodiment of a marking assembly 3700 is depicted. As shown, the backstop assembly 3716 may be coupled to a mounting plate 3726. The mounting plate 3726 may couple the marking assembly 3700 to a portion of an enclosure 12 of a system 10. The backstop assembly 3716 may include a guide body 3728 which may include a flange 3730. The embossing actuator 3714 may be coupled to the flange 3730. The embossing actuator 3714 may also be coupled to the mounting plate 3726 via a post 3732. The guide 3718 may be defined in the guide body 3728. In the example, the guide 3718 is arranged to accept and direct ports 392 of a bag 26 into position as the bag 26 is introduced to the marking assembly 3702. A guide 3718 may be a slot in the guide body 3728 which tapers from a widest point at its open end to a smaller width at an end opposite the opening. The taper on the guide 3718 may tend to lead a bag 26 into place and aid in straightening out the flexible material of the bag 26 at and in the vicinity of the target region to be marked. Example guides 3718 may also include a stepwise change in width. The tiered guide 3718 show in FIG. 201 may provide space to accept various features present on the port 392 which alter the outer diameter of the port 392 over its length.

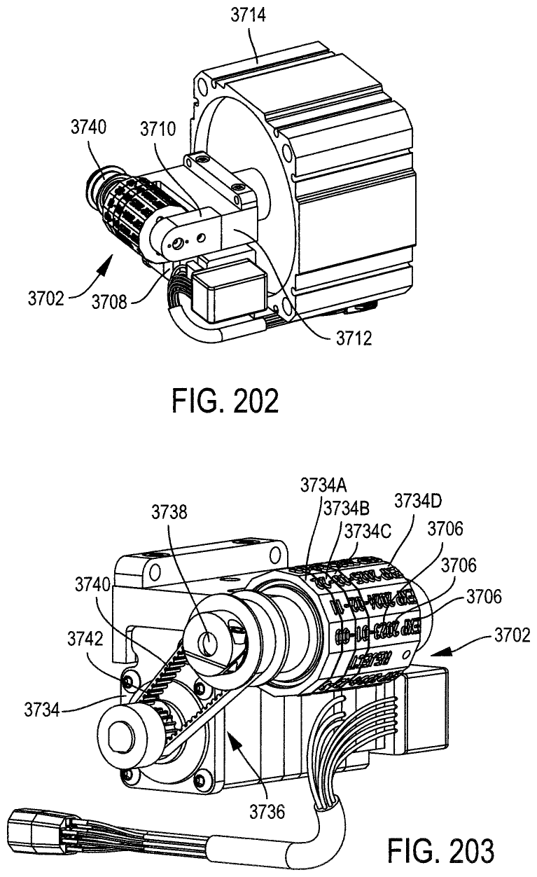

Referring now also to FIGS. 202-203, perspective views of the marking assembly 3700 of FIG. 201 with the backstop assembly 3716 and mounting plate 3726 removed are depicted. The embossing actuator 3714 is also removed in FIG. 203. As shown, various marking assemblies 3700 may include an embossing head 3702 which may be segmented into a number of embossing head rotors 3734A-D. Though four embossing head rotors 3734A-D are shown, any suitable number embossing head rotors 3734A-D may be included. The embossing head rotors 3734A-D may be constructed of a metallic, rigid, hard, durable material. In some examples 416 stainless steel may be used. The embossing head rotors 3734A-D may be the same width or at least one of the embossing head rotors 3734A-D may have a width which differs from companion rotors 3734A-D of the embossing head 3702. Each of the example embossing head rotors 3734A-D has a polygonal cross-section. As shown, the embossing head rotors 3734A-D are tridecagonal in cross-section, though any polygonal shape may be used. Each of the 13 faces of the exterior surface 3704 of each embossing head rotor 3734A-D may include one or more marking body 3706. Some faces of the exterior surface 3704 of each embossing head rotor 3734A-D may be nude, blank, or unadorned as shown in FIG. 203. In the example embodiment, the marking bodies 3706 include alphabetic, numeric, and punctuation characters. The embossing head rotors 3734A-D may be rotated such that the desired marking bodies 3706 are aligned at a marking position to create a desired string of characters for marking a bag 26. The face of the exterior surface 3704 of an embossing head rotor 3734A-D most distal the embossing actuator 3714 and normal to the displacement axis of the embossing head 3702 may be considered to be in the marking position.

For purposes of example, the marking bodies 3706 included on the depicted embossing head 3702 support the marking of an expiration date on the bag 26. The expiration date format is shown as “EXP YYYY-MM-DD”, though any desired date format could be used. Expiration dates may be calculated by the control system 15 based on the time point at which filling of the bag 26 began or completed. The expiration date may be a predefined amount of time after filling of the bag 26 began or completed. In some embodiments, the predefined amount of time may be specific to the solution the bag 26 contains.

The marking bodies 3706 may also include characters which may be arranged to mark the word “REJECT”, “DISCARD”, “NOT FOR HUMAN USE”, or the like on a bag 26 in the event the bag 26 is determined unacceptable (e.g., particulate has been determined to be present in the bag 26). Tables 1 and 2 below provide example marking body 3706 placements which may be included on embossing head rotors 3734A-D for an embossing head 3702. Preferably, the marking bodies 3706 may be positioned on the embossing head rotors 3734A-D such that force is substantially centered on the embossing head 3702 regardless of the character arrangement being used to mark. As depicted, for example, in FIG. 203, the “REJE” marking bodies 3706 on embossing rotor 3734A are justified proximal embossing rotor 3734B for this purpose.

TABLE 1

| Face | 3734D | 3734C | 3734B | 3734A |
|---|---|---|---|---|
| 1 | EXP(SPACE)2023- | 01 | −0 | 0 |
| 2 | EXP(SPACE)2024- | 02 | −1 | 1 |
| 3 | EXP(SPACE)2025- | 03 | −2 | 2 |
| 4 | EXP(SPACE)2026- | 04 | −3 | 3 |
| 5 | EXP(SPACE)2027- | 05 | −0 | 4 |
| 6 | EXP(SPACE)2028- | 06 | −1 | 5 |
| 7 | EXP(SPACE)2029- | 07 | −2 | 6 |
| 8 | EXP(SPACE)2030- | 08 | −3 | 7 |
| 9 | EXP(SPACE)2031- | 09 | −0 | 8 |
| 10 | EXP(SPACE)2032- | 10 | −1 | 9 |
| 11 | EXP(SPACE)2033- | 11 | −2 | (BLANK) |
| 12 | EXP(SPACE)2034- | 12 | −3 | (BLANK) |
| 13 | REJE | CT | (BLANK) | (BLANK) |

TABLE 2

| Face | 3734D | 3734C | 3734B | 3734A |
|---|---|---|---|---|
| 1 | REJEC | T | (BLANK) | (BLANK) |
| 2 | EXP(SPACE)2023- | 01 | −0 | 0 |
| 3 | EXP(SPACE)2024- | 02 | −1 | 1 |
| 4 | EXP(SPACE)2025- | 03 | −2 | 2 |
| 5 | EXP(SPACE)2026- | 04 | −3 | 3 |
| 6 | EXP(SPACE)2027- | 05 | −0 | 4 |
| 7 | EXP(SPACE)2028- | 06 | −1 | 5 |
| 8 | EXP(SPACE)2029- | 07 | −2 | 6 |
| 9 | EXP(SPACE)2030- | 08 | −3 | 7 |
| 10 | EXP(SPACE)2031- | 09 | −0 | 8 |
| 11 | EXP(SPACE)2032- | 10 | −1 | 9 |
| 12 | EXP(SPACE)2033- | 11 | −2 | (BLANK) |
| 13 | EXP(SPACE)2034- | 12 | −3 | (BLANK) |

The embossing head 3702 in FIGS. 202-203 includes marking bodies 3706 as specified in Table 1. As indicated in relation to rotor 3734B above, some rotors 3734A-D may have repeat, identical marking bodies 3706 on various faces of the exterior surface 3704. This may allow adjustments between desired strings of characters to be made more efficiently as multiple relative rotor positions may be acceptable to generate the same desired alignment of marking bodies 3706. Thus, less rotation may be needed to achieve a desired alignment of marking bodies 3706. In some embodiments, marking bodies 3706 which are rarely used (e.g. “3” for the tens place of a month) may be included less frequently on a rotor 3734A-D. Certain faces of rotors 3734A-D may also be left blank. Additionally, repeat marking bodies 3706 may increase life of the embossing head 3702. Where certain marking bodies 3706 are used with high frequency, it may be desired that duplicates be present. This may facilitate use of an alternative, equivalent marking body 3706 in the event a given marking body 3706 is determined to have reached a maximum defined number of embossing cycles. In some embodiments, the number of embossing cycles for each face of each rotor 3734A-D may be tracked and incremented by the control system 15 with each embossing operation. The control system 15 may use an alternative, equivalent marking body 3706 in the event that embossing cycles for another marking body 3706 is in breach of a predefined maximum threshold.

Still referring to FIGS. 202-203, a marking assembly 3700 may include a rotary actuator 3708 which may be powered to displace the various embossing head rotors 3734A-D of the embossing head 3702. As described in greater detail below, the rotary actuator 3708 may displace the embossing head rotors 3734A-D in tandem or may displace at least one embossing head rotor 3734A-D relative to another. A rotary position sensor 3724 may be included to monitor the position of a shaft 3738 on which the embossing head rotors 3734A-D are disposed. The rotary position sensor 3724 may be a rotary encoder or absolute rotary encoder monitoring the output of the rotary actuator 3708 in certain examples. In some embodiments, a rotary position sensor 3724 may instead or additionally directly monitor the rotational position of the shaft 3738. In certain examples, a transmission 3736 may be disposed intermediate the rotary actuator 3708 and the shaft 3738. Any transmission 3736 may be utilized and may include one or more gears, belts, and pulleys. In the example, a timing belt 3740 is included. The output of the rotary actuator 3708 and the shaft 3738 may each include a teethed region 3742 which may interdigitate with the teeth of the timing belt 3740.

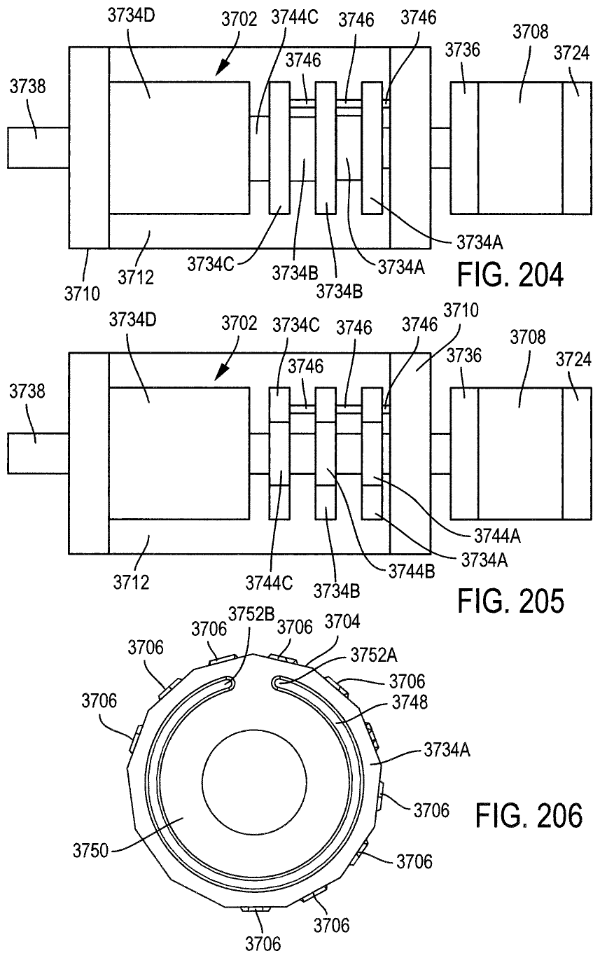

Referring now to FIGS. 204-205, two block diagrams of embossing heads 3702 are depicted. The embossing heads 3702 are operatively coupled to a rotary actuator 3708, optionally via a transmission 3736. A rotary positon sensor 3724 may be included and may generate a signal that varies in relation to the rotational position of the shaft 3738 on which each of the embossing head rotors 3734A-D is disposed. As shown, all but one of the embossing head rotors 3734A-D are paired with a clutch 3744A, B, C. The remaining embossing head rotor (rotor 3734D in the examples) may be fixedly retained on the shaft 3738 and rotate therewith. The clutches 3744A, B, C may be slip clutches. The example embodiment shown in FIG. 204 includes axial clutches 3744A, B, C which interact with adjacent embossing head rotors 3734A-D to constrain pairs of embossing head rotors 3734A-D to rotate in tandem. The example embodiment illustrated in FIG. 205 includes radial clutches 3744A, B, C. The radial clutches 3744A, B, C may constrain respective embossing head rotors 3734A-D to rotate in tandem with the shaft 3738. In either embodiment, the clutches 3744A, B, C may be made to slip to engender relative motion between an embossing head rotor 3434A-D and one or more of the companion embossing head rotors 3734A-D (and shaft 3738). Preferably, the clutches 3744A-C may generate minimal or no particulate.

Adjacent embossing head rotors 3734A-D may form a relative rotation limiter between them when the embossing head 3702 is assembled. The relative rotation limiter may prevent relative rotation between the adjacent rotors 3734A-D beyond a maximum amount in both the clockwise and counterclockwise directions. In certain examples, this may be established by a mechanical interference which is engaged as the relative rotation limit in the clockwise or counterclockwise direction has been reached. Slippage of clutches 3744A-C may occur once relative rotation has been inhibited by a respective relative rotation limiter.

Referring now also to FIG. 206, a view of a side face 3750 of an example embossing head rotor 3734A is depicted. Embossing head rotors 3734A-D may include an interrupted annular track 3748 in at least one side face 3750. In various embodiments, the interrupt may be present for an arc of (360°/n), where “n” is the number of side faces on the exterior surface 3704 of the embossing head rotors 3734A-D. The interrupted annular track 3748 may be a continuous track and may have first and second ends 3752A, B. Each interrupted annular track 3748 may span from a point aligned with the center a first face of the exterior surface 3704 of a respective embossing head rotor 3734 to a second point aligned with the center of the last or “n”th face (adjacent the first face) of the embossing head rotor 3734A-D. Embossing head rotors 3734A-C may also include a pin 3746 projecting from at least one side face 3750. Any pins 3746 may extend into and travel along the interrupted annular track 3748 of an adjacent embossing head rotor 3734A-C. The interaction of the pin 3746 of one embossing head rotor 3734A-D with the interrupted annular channel 3748 of the adjacent embossing head rotor 3734A-D may form an example of a relative rotation limiter.

A pin 3746 may also project from the trunnion 3710 adjacent at least one of the embossing head rotors 3734A-D. Alternatively, the trunnion 3710 may include an interrupted annular track 3748. In such examples, the adjacent embossing head rotor 3734A may include a pin 3746 which travels within the interrupted annular track 3748 of the trunnion 3710. The pin 3746 or interrupted annular track 3748 in the trunnion 3710 may be stationary.

For sake of brevity, the following description refers to embodiments including a pin 3746 in the trunnion 3710 which rides along an interrupted annular channel 3748 in the adjacent rotor 3734A. In alternative embodiments, the annular interrupted channel 3748 may be in the trunnion 3710 and the pin 3746 may be disposed in the adjacent rotor 3734A. Similarly, where a pin 3746 is described as extending from an embossing head rotor 3734A-C to the interrupted annular track 3748 of an adjacent embossing head rotor 3734A-C, this is exemplary. The interrupted annular track 3748 could be included in the embossing head rotor 3734A-C and the pin 3746 could be disposed in the adjacent embossing head rotor 3734A-C instead in alternative embodiments.

As the shaft 3738 is rotated in a first direction, the embossing head rotors 3734A-D may rotate in tandem with the shaft 3738. One embossing head rotor 3734D may be fixedly retained on the shaft 3738. The other embossing head rotors 3734A-C may rotate in tandem with the shaft 3738 due to the presence of the clutches 3744A-C. The pins 3746 may be disposed in the interrupted annular tracks 3748 of the adjacent embossing head rotor 3734A-D (or trunnion 3710). As the pin 3746 in the trunnion 3710 is stationary, it may ride along the interrupted annular track 3748 of the adjacent rotor 3734A as rotation of the rotors 3734A-D occurs.

Eventually, as the shaft 3738 continues to rotate in the first direction, the pin 3746 of the trunnion 3710 may reach an end 3752A, B of the interrupted annular channel 3748 in the rotor 3734A adjacent the trunnion 3710. This may present a mechanical interference to additional rotation of the rotor 3734A (sometimes referred to herein as first rotor 3734A) as the shaft 3738 rotates further in the first direction. The relative rotation limiter may be said to be in an engaged state in the first rotation direction for the rotor 3734A adjacent the trunnion 3710 at this point. The clutch 3744A paired with the first rotor 3734A may slip allowing the shaft 3738 to continue to rotate in the first direction in tandem with the other rotors 3734B-D.

As the first rotor 3734A is rendered stationary by engagement of the respective relative rotation limiter, further rotation of the shaft 3738 in the first direction may eventually engage the relative rotation limiter between the first rotor 3734A and second rotor 3734B. Thus, the relative rotation limiter may be in an engaged state in the first rotation direction for the second rotor 3734B. In various examples, a pin 3746 extending from the second rotor 3748B may be displaced along the interrupted annular track 3748 in the adjacent face 3750 of the first rotor 3734A. This may occur until the pin 3746 is rotated into contact with an end 3752A, B of the interrupted annular track 3748 preventing further relative rotation of the second rotor 3734B relative to the first rotor 3734A. The clutch 3744B paired with the second rotor 3734B may slip allowing the shaft 3738 to continue to rotate in the first direction in tandem with rotors 3734C-D.

With the first and second rotors 3734 A, B rendered stationary by engagement of the respective relative rotation limiters, further rotation of the shaft 3738 in the first direction may eventually engage the relative rotation limiter between the second rotor 3734B and third rotor 3734C. Thus, the relative rotation limiter may be in an engaged state in the first rotation direction for the third rotor 3734B. In various examples, an interrupted annular track 3748 of third rotor 3748B may be displaced over a stationary pin 3746 extending from the adjacent side face 3750 of the second rotor 3734A. This may occur until an end 3752A, B of the interrupted annular track 3748 contacts the pin 3746 preventing further relative rotation of the third rotor 3734C relative to the second 3734B. The clutch 3744C paired with the third rotor 3734C may slip allowing the shaft 3738 to continue to rotate in the first direction in unison with the fourth rotor 3734D.

With the relative rotation limiters of the first, second, and third rotors 3734A-C in engaged states, the remaining fourth rotor 3734D may rotate in tandem with the shaft 3738 as the shaft 3738 continues to rotationally displace in the first direction. The fourth rotor 3734D may not have an associated relative rotation limiter. As the remaining rotor 3734D rotates, the relative position of the fourth rotor 3734D may be specified relative to the third rotor 3734C. Once this relative position is specified, the shaft 3738 may then be rotated in a second direction opposite the first direction. As relative rotation limiters are engaged in the first rotation direction, rotation in the second direction may be permitted. The rotors 3734A-D may all rotate in tandem with the shaft 3738 in the second direction.

Eventually, the pin 3746 in the trunnion 3710 may contact the opposite end 3752A, B of the interrupted annular track 3748 of the first rotor 3734A. This may present a mechanical interference to additional rotation of the first rotor 3734A as the shaft 3738 rotates further in the second direction. The relative rotation limiter may be said to be in an engaged state in the second rotation direction for the rotor 3734A adjacent the trunnion 3710 at this point. The clutch 3744A paired with the first rotor 3734A may slip allowing the shaft 3738 to continue to rotate in the second direction in tandem with the other rotors 3734B-D.

With the first rotor 3734A being rendered stationary by engagement of the respective relative rotation limiter, further rotation of the shaft 3738 in the second direction may eventually engage the relative rotation limiter between the first rotor 3734A and second rotor 3734B. Thus, the relative rotation limiter may be in an engaged state in the second rotation direction for the second rotor 3734B. In various examples, a pin 3746 extending from the second rotor 3748B may displace along the interrupted annular track 3748 in the adjacent face 3750 of the first rotor 3734A. This may occur until the pin 3746 is rotated into contact with an end 3752A, B of the interrupted annular track 3748 preventing further relative rotation of the second rotor 3734B relative to the first 3734A. The clutch 3744B paired with the second rotor 3734B may slip allowing the shaft 3738 to continue to rotate in the second direction in tandem with rotors 3734C-D. As the first and second rotors 3734A, B are rendered stationary, further rotation of the shaft 3738 in the second direction may set the desired position of the third rotor 3734C relative to the second rotor 3734B.

The rotation direction of the shaft 3738 may be reversed until the relative rotation limiter for the first rotor 3734A is again engaged in the first rotation direction. Continued rotation in the first direction may establish a desired relative position of the second rotor 3734B to the first rotor 3734A. Thus, the marking bodies 3706 across the exterior surfaces 3704 of the embossing head rotors 3734A-D may be aligned in a desired character string. The shaft 3738 may then be rotated in the second direction. All rotors 3734A-D may be rotated in tandem with the shaft 3738 until the desired character string is disposed in the marking position. An embossing actuator 3714 may then be driven to displace the embossing head 3702 against a bag 26, other reservoir, or work piece.

The example embossing head 3702 may also be driven to a home position if necessary. The home position may provide a known position of each rotor 3734A-D relative to one another. Where the final rotor 3734D is fixed relative to the shaft 3738, the rotary position sensor 3724 may directly track to the position of the final rotor 3734D. The control system 15 may command the rotary actuator 3708 to rotate the shaft 3738 a predetermined amount. This amount may be a number of degrees which ensures that the relative rotation limiters for each rotor 3734A-D have been engaged in that rotation direction. As the rotation position of a given rotor 3734A-D may be known when the respective relative rotation limiter is engaged, this may allow the control system 15 to establish a known starting position for all rotors 3734A-D. This may, for example, be commanded by the control system 15 upon installation of a new embossing head 3702 or after an interruption of power to the system 10.

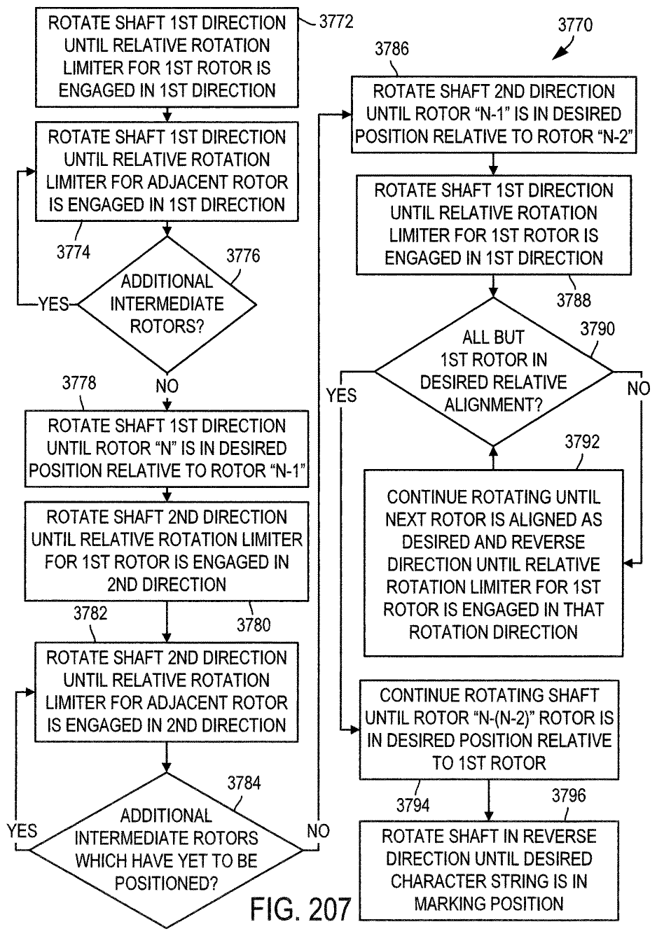

Referring now to FIG. 207, a flowchart 3770 depicting a number of example actions which may be executed to displace desired marking bodies 3706 on rotors 3734A-D of an embossing head 3702 into a marking position is depicted. Though four rotors 3734A-D are shown in the example embodiment, embossing heads 3702 may include a lesser or greater number of rotors 3734A-D in alternative embodiments. The flowchart 3770 describes placing desired marking bodies 3706 in position for an embossing head 3702 with “n” rotors where “n” is any desired number of rotors. Rotors of interest are described in relation to “n” within the flowchart 3770. For example, rotor “n−1” would be adjacent to rotor “n” and would be the penultimate rotor. Rotor “n−(n−2)” would be the rotor second most distal to rotor “n” and adjacent the first rotor of the embossing head 3702. As with various examples described herein, rotor “n” is fixed relative to the shaft 3738.

As shown, in block 3772 the shaft 3738 may be rotated in a first direction until a relative rotation limiter for the first rotor of the embossing head 3702 is engaged in the first direction. The first rotor may be rendered stationary relative to the trunnion 3710 with further rotation of the shaft 3738 in the first direction. The shaft 3738 may continue to be rotated in the first direction until a relative rotation limiter for the adjacent rotor (the rotor next most distal to rotor “n”) is engaged in the first direction in block 3774. If there are additional intermediate rotors (those between the first and last rotor) in block 3776, block 3774 may repeat. Once there are no further intermediate rotors in block 3776, the shaft 3738 may continue to be rotated in the first direction in block 3778 until the last rotor (rotor “n”) is in a desired position relative to the penultimate rotor (rotor “n−1”). The final rotor and the penultimate rotor may be considered to be positioned once block 3778 has completed. In block 3780, the shaft 3738 may be rotated in a second direction opposite the first direction until a relative rotation limiter for the first rotor is engaged in the second direction. The shaft 3738 may continue to be rotated in the second direction until a relative rotation limiter for the adjacent rotor (the rotor next most distal to rotor “n”) is engaged in the second direction in block 3782. If, in block 3784, there are additional intermediate rotors which have yet to be positioned, block 3782 may repeat. Once all intermediate rotors which were yet to be positioned have had their relative rotation locks engaged in block 3784, the shaft 3738 may continue to be rotated in the second direction until rotor “n−1” is in a desired relative position to rotor “n−2” in block 3786. In block 3788, the shaft 3738 may be rotated in the first direction until the relative rotation limiter for the first rotor is engaged in the first direction. If there are additional rotors (other than the first rotor) to be positioned in block 3790, rotation of the shaft 3738 may continue until the next rotor (the most proximal unpositioned rotor with respect to rotor “n”) is in the desired relative alignment with respect to the positioned rotors in block 3792. Also in block 3792, the rotation direction of the shaft 3738 may be reversed and the shaft 3738 may rotate until the relative rotation limiter for the first rotor is engaged in that rotation direction. Block 3792 may repeat until all but the first rotor have been positioned in block 3790. If, in block 3790, all but the first rotor are positioned as desired, the shaft 3738 may continue to be rotated until the “n−(n−2)” rotor is in the desired position relative to the first rotor in block 3794. The first rotor may be considered to be positioned at this point. With all rotors positioned, a desired string of characters to emboss will be in alignment with one another. The rotation direction of the shaft 3738 may be reversed and the rotors may be rotated until the desired character string is in the marking position in block 3796.

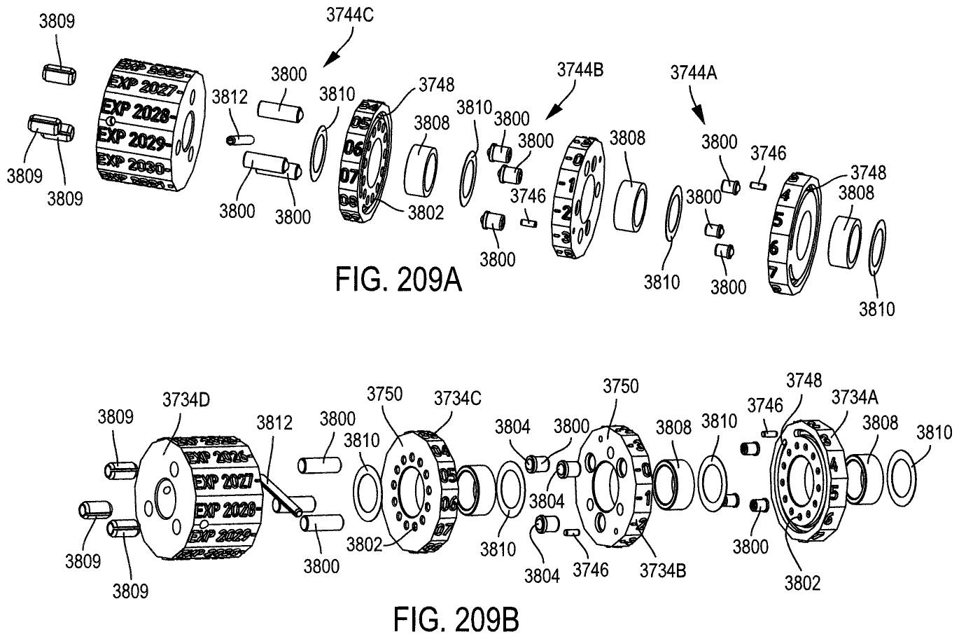

Referring now to FIGS. 208A-209B, exploded views of an example embossing head 3702 are depicted. An example trunnion 3710 and base 3712 are depicted in FIGS. 208A-B as well. The example embossing head 3702 of FIGS. 209A-B is axially clutched. The final rotor 3734D is fixedly retained on the shaft 3738 by a pin 3812. In the example embodiment, the clutches 3744A-C of the embossing head 3702 each include at least one spring plunger 3800. The spring plungers 3800 may be ball spring plungers. As shown, the spring plungers 3800 may be disposed within certain of the rotors 3734A-D and be oriented parallel to the axial dimension of a rotor 3734A-D in which they are housed. The spring plungers 3800 may be coupled or anchored into respective rotors 3734A-D in any suitable fashion. In some examples, retaining compound may be used. Alternatively, the spring plungers 3800 may be provided with a flange having a diameter larger than at least a portion of a receiving orifice for the spring plunger 3800 in a given rotor 3734A-D. In such examples, the spring plungers 3800 may be held in place by the compression in the rotor stack. In certain examples, a spring pin may be introduced to the opposing side of a rotor 3734A-D to set the depth of the spring plunger 3800 within a given rotor 3734A-D (see, e.g. rotor 3734D of FIG. 209B). The side face 3750 of the adjacent rotor 3734A-D may include a set of detents 3802. The plungers 3804 of the spring plungers 3800 may be biased in a direction normal to the side face 3750 of the rotor 3734A-D in which they are housed. When the embossing head 3702 is assembled, the plungers 3804 may be biased into contact with the side face 3750 of the adjacent rotor 3734A-D. As relative rotation between the rotor 3734A-D housing the spring plungers 3800 and the adjacent rotor 3734A-D occurs, the detents 3802 may come into alignment with the plungers 3804. The plungers 3804 may be urged into the detents 3802 by the bias exerted by the spring 3820 (see, e.g., FIG. 211) of the spring plungers 3800. The detents 3802 may have a depth which is insufficient to permit the plungers 3804 to reach a resting state. This may ensure the plungers 3804 may be firmly held in position in the detents 3802 by the spring bias of the spring plungers 3800.

The detents 3802 may be spaced at even angular increments. Where the rotor 3734A-D has a polygonal cross section with “n” sides, the spacing may be 360°/“n”. The transition from the side face 3750 to the detents 3802 may also be contoured such that the spring bias of the spring plungers 3800 tends to self-center the plungers 3804 in respective detents 3802. In the example embodiment, each clutch 3744A-C includes three spring plungers 3800. The number of spring plungers 3800 may differ in alternative examples. The spring plungers 3800 may be spaced at even or approximately even angular increments in order to help balance forces. Where the number of detents 3802 is not a multiple of the number of spring plungers 3800 in a given clutch 3744A-C, exact even spacing will not be possible. The spring plungers 3800 may be considered approximately evenly spaced when the spring plungers 3800 are spaced as close to even as the number of detents 3802 permit. The pins 3746 and interrupted annular tracks 3748 may be spaced radially inboard or outboard (as shown) with respect the detents 3802 and spring plungers 3800.

With plungers 3804 of a rotor 3734A-D pressed into respective detents 3802 of an adjacent rotor 3734A-D, relative rotation between the rotors 3734A-D may require sufficient force to overcome the bias of the spring plungers 3800 to depress the plungers 3804. Thus, the spring plungers 3800 and detents 3802 may act as a clutch. The rotors 3734A-D may tend to rotate in tandem with one another until a relative rotation limiter is engaged. With one of the rotors 3734A-D inhibited from further rotation, its respective clutch (provided by the interaction of the spring plungers 3800 and detents 3802) may slip as increasing force is applied to drive rotation of the shaft 3738. The embossing head 3702 may include a series of clutches which get progressively stronger as proximity to the final or “n”th rotor 3734D increases. This may ensure that the clutches slip in a prescribed order as the string of marking bodies 3706 is brought into a desired alignment. Strength of the axial clutches may be adjusted by altering the spring plunger 3800 (e.g. by adjusting the strength of the spring 3820). Alternatively or additionally, the strength of the clutches may be adjusted by altering the lead in angle to the detents 3802. The depth of the detents 3802 and/or the distance the plungers 3804 protrude from their respective rotor 3734A-D may be adjusted to alter the strength of a clutch. Sharper angles leading into the detents 3802 may increase the slip resistance of the respective clutch. The number of spring plungers 3800 may also be adjusted to alter the strength of a given axial clutch of the embossing head 3702. The radial distance from the rotation axis may also be adjusted. The greater the distance from the rotation axis, the more torque required to slip the clutch. In general, the force required to slip a particular clutch may be chosen to be less than that require to depress a plunger 3804 of the next weakest clutch. Thus, plungers 3804 of the next weakest clutch may remain engaged with respective detents 3802 while motion relative to the weaker clutch occurs. This may ensure deterministic movement of the rotors 3734A-D.

As shown, the embossing head 3702 includes a collet 3739. The collet 3739, in conjunction with the rotor 3734D fixedly coupled to the shaft 3738 may be used to ensure the rotor stack is under a desired amount of compression after assembly. The shaft 3738 may be passed through a portion of the trunnion 3710 such that the rotors 3734A-D are on a first side of the portion of the trunnion 3710 and a section of the shaft 3738 protrudes from an opposing side of the trunnion 3710. The protruding portion of the shaft 3738 may be displaced so as to protrude a greater distance from that side of the trunnion 3710 portion. Rotor 3734D may also be displaced as this occurs. This may cause the clutches 3744A-C to be placed under compression. The collet 3739 may then be tightened on the shaft 3738 locking the shaft 3738 position relative to the trunnion 3710. In some embodiments, a shim of a desired thickness may be placed in the rotor stack as the shaft 3738 position is adjusted. The shim may be removed after tightening the collet 3739 on the shaft 3738 into a clamping state. The gap created by the removal of the shim may propagate to the strongest clutch. In the example, the gap would propagate to the clutch between rotor 3734C and rotor 3734D.

Still referring to FIGS. 208A-209B, the trunnion 3710 may include bearings 3806 for the shaft 3738. A rotor bearing 3808 may also be included for the rotors 3734A-C not fixed to the shaft 3738. Each rotor 3734A-D may be separated from any adjacent components (e.g. rotors 3734A-D or trunnion 3710) by a thrust bearing 3810. In some examples, thrust bearings 3810 may be PTFE washers.

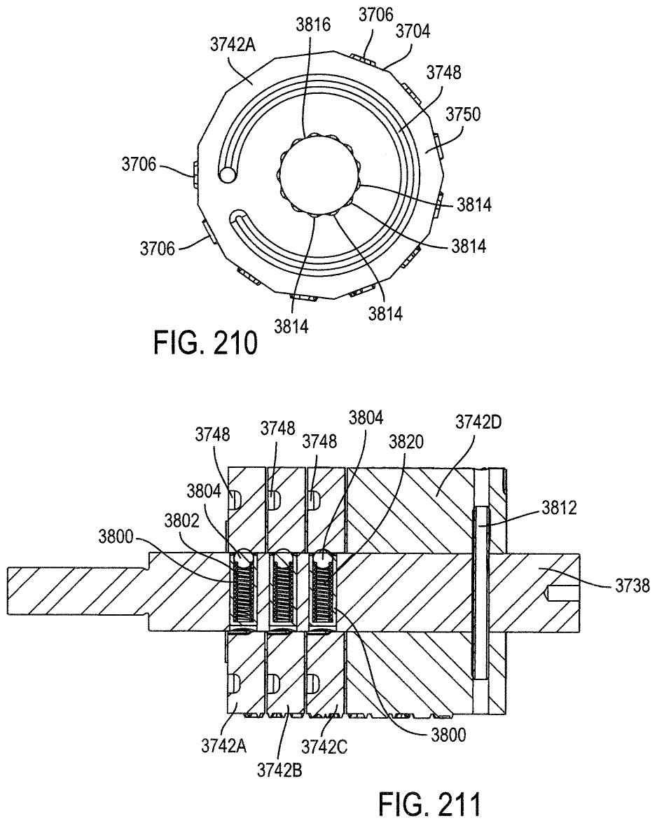

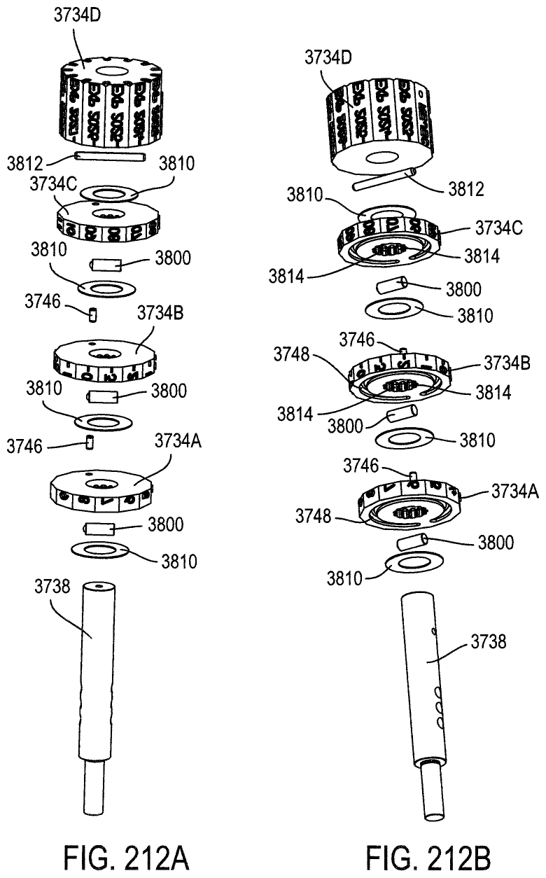

Referring now to FIGS. 210-212B, an example rotor 3734A and embossing head 3702 are depicted. FIG. 210 depicts a side view of an example rotor 3734A. A cross-sectional view of an embossing head 3702 is depicted in FIG. 211. Exploded views of the example embossing head 3702 are shown in FIGS. 212A-B. The example embossing head 3702 of FIGS. 211-212B is radially clutched with the final rotor 3734D fixedly retained on the shaft 3738 by a pin 3812.

As best shown in FIG. 210, the side face 3750 of the example rotors 3734A-D may be devoid of detents 3802. Instead, the radial detents 3814 may be included in the aperture 3816 of each rotor 3734A-D through which the shaft 3738 extends. In certain examples, the radial detents 3814 may be formed as depressions which extend from a first side face 3750 and partially toward the opposing side face 3750 (best shown in FIG. 212A). The number of radial detents 3814 may be equal to the number of faces of a rotor's 3734A-D cross-sectional shape. The shaft 3738 may include a number of spring plungers 3800 disposed therein. The plungers 3804 of the spring plungers 3800 may be proud of the surface of the shaft 3738. In the example, the plungers 3804 are balls.

In certain examples, each rotor 3734A-D that rotates relative to the shaft 3738 may include a bushing insert for the shaft 3738. The radial detents 3814 may be provided in the bushing insert. The bushing insert may be made of a self-lubricating material. An oil containing bronze or other metallic material (e.g. Oilite) may be used in certain examples. In some embodiments, the bushing inserts may extend past each side of the parent rotor 3734A-D and may also serve as thrust bearings in certain implementations. A single sleeve bushing may also be placed over the shaft 3738 and may be accepted by the aperture 3816 of each rotor 3734A-D in alternative embodiments. The plungers 3804 may extend through and be proud of the sleeve bushing in embodiments where a sleeve bushing is used.

The springs 3820 of the spring plungers 3800 may bias the plunger 3804 into a radial detent 3814 of the paired rotor 3734A-D. The spring 3820 of each spring plunger 3800 may be under compression when the respective plunger 3804 is disposed in a radial detent 3814. This, in conjunction with the lead in to the radial detents 3814, may assist in self-centering the radial detent 3814 on the plunger 3804. When a plunger 3804 is disposed in a radial detent 3814, the rotor 3734A-D may rotate in tandem with the shaft 3738 until a relative rotation limiter is engaged in that direction. Thus, the spring plungers 3800 and radial detents 3814 may form radial clutches. Once a relative rotation limiter is engaged, further rotation of the shaft 3738 may cause slippage of the respective radial clutch.

The example embossing heads 3702 depicted in FIGS. 208A-212B may be particularly compact with minimal gap between the rotors 3734A-D. In some examples, the gap may be 0.011″ or less between rotors 3734A-D. This may allow for a maximized font size to be utilized with a given target impression area on a work piece such as a bag 26. Additionally, in various embodiments, the rotors 3734A-D may be under rigid as well as spring compression. This may inhibit canting of the rotors 3734A-D and help to ensure individual rotors 3734A-D remain out of contact with one another despite the tight spacing.

Referring now to FIG. 213A, in certain examples, a marking assembly 3700 may be paired with a mark verification assembly. The mark verification assembly may include at least one imager 3733. The imager 3733 may be positioned such that the marked region of the bag 26 (or other reservoir) is in the field of view of the imager 3733. Alternatively, the bag 26 may be gripped by a gripper assembly 4204 of a gantry assembly 4200 and the marking on the bag 26 may be displaced into the field of view of the imager 3733. As shown, the marking is in the enlarged region 36A of the peripheral seal 30 of the example bag 26. The marking could alternatively be placed in an uncoupled region 60. An image 3735 may be captured of the marking. Preferably, the imaged side of the bag 26 may be the side opposite that contacted by the embossing head 3702. One or more light emitter may be positioned to backlight the portion of the bag 26 including the marking. In some embodiments, a lighting panel may, for example, be mounted to an interior wall of the enclosure and the bag 26 may be displaced between the imager 3733 and lighting panel during mark verification.

A representation of such an image 3735 is depicted in FIG. 213B. The raw image may be pre-processed (e.g. subjected to binary thresholding, despeckled, de-skewed, etc.). An optical character recognition algorithm may then be used to determine the content of the marking on the bag 26. Any suitable optical character recognition algorithm such as a matrix matching or feature extraction type algorithm may be used. Alternatively, the image 3735 may be provided to a neural network trained on images of markings on bags 26 made from each of the marking bodies 3706 in the marking assembly 3700. Any character recognition may be constrained by a lexicon or dictionary of potential markings built from the marking bodies 3706 present in the marking assembly 3700. In the event that the determined marking from the image 3735 matches the desired mark, the bag 26 may be collected by a gantry assembly 4200 and displaced to an appropriate receptacle 4120A, B in an outfeed drawer 4102 for example. In the event that the determined marking does not match the desired mark (or the control system 15 cannot determine the content of the marking in the image 3735), the bag 26 may be re-marked at least one time by the marking assembly 3700. The subsequent marking(s) may indicate the bag 26 is rejected or may make any marking on the bag 26 unintelligible. In the latter scenario, several different markings may be made on top of any existing mark. The bag 26 may then be displaced to an access-controlled receptacle 4120B of an outfeed assembly 4100. Alternatively, the marking may be rendered unintelligible as describe above and the bag 26 may be removed from the marking assembly 3700. The bag 26 may be rotated 180°, the opposing unmarked side of the bag 26 may be advanced into a marking assembly 2700, and an unmarked portion of the bag 26 on the opposing side may be marked to indicate the bag 26 is rejected. This marking may also be verified the control system 15 via data from the mark verification assembly as described above.

Referring now to FIG. 213C, an annotated image 3737 may be generated in certain embodiments. The annotated image 3737 may include an indicator 3741 around each character recognized in the optical character recognition analysis. The indicator 3741 may be representative of the region analyzed for character recognition. A superimposed character determination 3743 indicative of the character determined to be present in the indicated region may be associated with each indicator 3741 in the annotated image 3737. A marking boundary 3745 indicative of where within the image the marking on the bag 26 was determined to be may also be included. An annotated image 3737 may be saved and provided in a log file associated with the unique identifier for each respective bag 26. Such log files may, for example, be stored on a database 3502.

A notification may be generated by the control system 15 in the event that a marking does not match the desired mark. In certain examples, the control system 15 may place the system 10 in a standby state until the marking assembly 3700 is serviced. Alternatively, the control system 15 may determine which marking body or bodies 3706 on which rotor(s) 3742A-D left marks that were unable to be verified based on data from the marking verification assembly. The control system 15 may check if these flagged marking bodies 3706 are redundant or duplicative of another marking body 3706 on the same parent rotor 3742A-D. The control system 15 may adjust relative displacement commands to the rotors 3742A-D to avoid use of the flagged marking body 3706 in favor of a redundant marking body 3706 on the parent rotor 3742A-D.

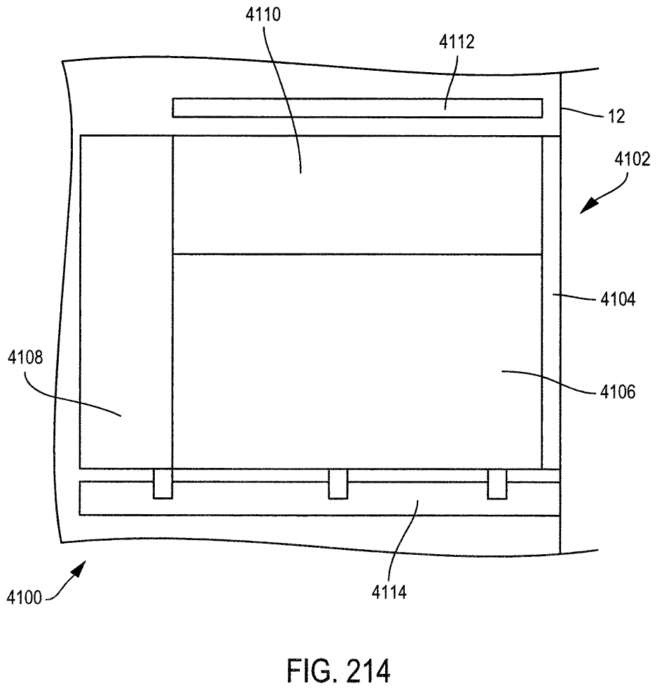

Referring now to FIG. 214, a block diagram of an example outfeed assembly 4100 is depicted. The example outfeed assembly 4100 may be included within an enclosure 12 of a system 10. The outfeed assembly 4100 may receive filled bags 26 which have been labeled for use and passed any required quality criteria. For example, bags 26 may pass one or more inspection described herein. Fluid dispensed into the bag 26 may also be required to have conformed to acceptability characteristics (e.g. predefined ranges for conductivity, temperature, etc.). The outfeed assembly 4100 may also receive and segregate filled bags 26 which have been rejected due to nonconformity to quality criteria. Acceptable and rejected bags 26 may be dispensed from the outfeed assembly 4100 with respective differing levels of access control. The outfeed assembly 4100 may prevent a typical user from physically accessing rejected bags 26. A specifically credentialed user may be required to collect rejected bags 26 from the system 10.

As shown in FIG. 214, an example outfeed assembly 4100 may include one or more outfeed drawer 4102. Each outfeed drawer 4102 may include an exterior panel 4104. The exterior panel 4104 may be flush with the exterior wall of the enclosure 12 when the outfeed drawer 4102 is in a closed state. A seal may be formed between the exterior panel 4104 and the enclosure 12 when the outfeed drawer 4102 is closed. A gasket or other sealing member may be coupled to the exterior panel 4104 for this purpose. A handle 4164 or other grasp features may be included on the exterior panel. Each outfeed drawer 4102 may include a first receptacle array 4106 and a second receptacle array 4108. The first receptacle array 4106 may include a plurality of receptacles 4120A (see, e.g., FIG. 216) sized to accept bags 26. The second receptacle array 4108 may also include a plurality, albeit smaller number, of receptacles 4120B (see, e.g., FIG. 221) for bags 26. Each outfeed drawer 4104 may be tiered such that the second receptacle array 4108 is raised or recessed with respect to the first receptacle array 4106. A transition wall 4118 may be present and form the step in height between the receptacle arrays 4106, 4108. The openings of the second receptacle array 4108 may be substantially even with the sidewall 4110 of the outfeed drawer 4102. The openings to the first receptacle array 4016 may be spaced from the top of the sidewall 4110 and recessed within the outfeed drawer 4102. The opposite may be true where the second receptacle array 4108 is recessed with respect to the first receptacle array 4106. In some embodiments, each receptacle array 4106, 4108 may be at even height and no tiered arrangement may be present (see, e.g., FIG. 224). The first receptacle array 4106 may hold bags 26 which have been deemed acceptable for use. The second receptacle array 4108 may hold bags 26 which have been rejected. In certain embodiments, the first receptacle array 4106 may include twenty-five receptacles 4120A in a number of rows and the second receptacle array 4108 may include three receptacles 4120B in a single row. Other arrangements are possible. The receptacle arrays 4106, 4108 in each outfeed drawer 4102 may be mirrored with respect to one another. In some examples, the receptacles 4120A may be a first color (e.g. white or green) while the receptacles 4120B may be second color (e.g. red). In some embodiments, all receptacles 4120A, B may be black or fully saturated colors (e.g. red, green, blue). This may assist in imaging based verification of outfeed drawer 4102 inventories. One or more faceplate may be included to cover all but the opening to the receptacles 4120A, B in each drawer 4102. The color of the one or more faceplates may differ depending on the surrounding receptacles 4120B. Faceplate regions adjacent the receptacles 4120A of the first array 4106 may be a first color (e.g. green). Those surround the receptacles 4120B of the second array 4108 may be a second color (e.g. red).

As shown, a warding plate or grid 4112 may be included in some examples. The warding grid 4112 may include apertures which align with the receptacles 4120A, B in the outfeed drawers 4102 when the outfeed drawers 4102 are in a closed state. Bags 26 may be dropped through the apertures in the warding grid 4112 into desired receptacles in the outfeed drawer 4102. When an outfeed drawer 4102 is in an open state, the warding grid 4112 may help inhibit a user from reaching into the enclosure 12. The apertures may be tapered to assist in directing bags 26 into the target receptacle 4120A, B.

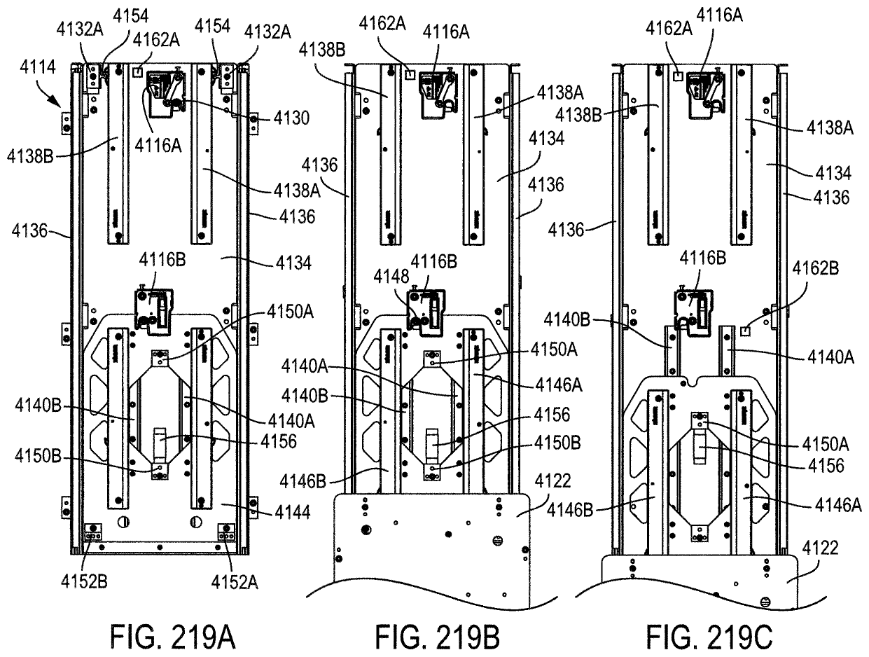

Each outfeed drawer 4102 may be coupled to a drawer slide assembly 4114. The drawer slide assembly 4114 may facilitate translational displacement of the associated outfeed drawer 4102 with respect to the enclosure 12. Each drawer slide assembly 4114 may include a plurality of lock assemblies 4116A, B (see, e.g., FIG. 219A). The lock assemblies 4116A, B may be actuated between a displacement permitting state and a displacement inhibiting state.

A user may present a credential (e.g. RFID badge, biometric such as a fingerprint, etc.) which is read by an access control assembly of the system 10 (RF interrogator, finger print scanner, etc.). Alternatively or additionally, a user may key in a user identity and associated password on a user interface of the system 10 (e.g. GUI 6100 of FIG. 1). An access request may be input to the system 10 (e.g. via a button press) and one or more of the lock assemblies 4116A, B may be actuated to a displacement permitting state. The type of credential presented and verified by the system 10 may determine which lock assemblies 4116A, B may be actuated by the user. A standard user (e.g. pharmacist, nurse, or other medical practitioner) may be provided credentials that open a first lock assembly 4116A, B. Additional lock assemblies 4116A, B may require specialized credentials which may, for example, only be issued to system technicians or personnel with specific training. In some examples, the control system 15 may prohibit the lock assemblies 4116A, B from being in a displacement permitting state during at least one system 10 state. The control system 15 may prevent actuation of the lock assemblies 4116A, B of an outfeed drawer 4102 to a displacement permitting state until at least the first receptacle array 4106 is filled at least partially with bags 26.

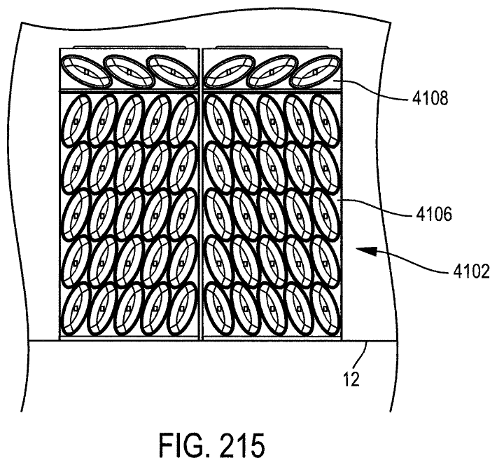

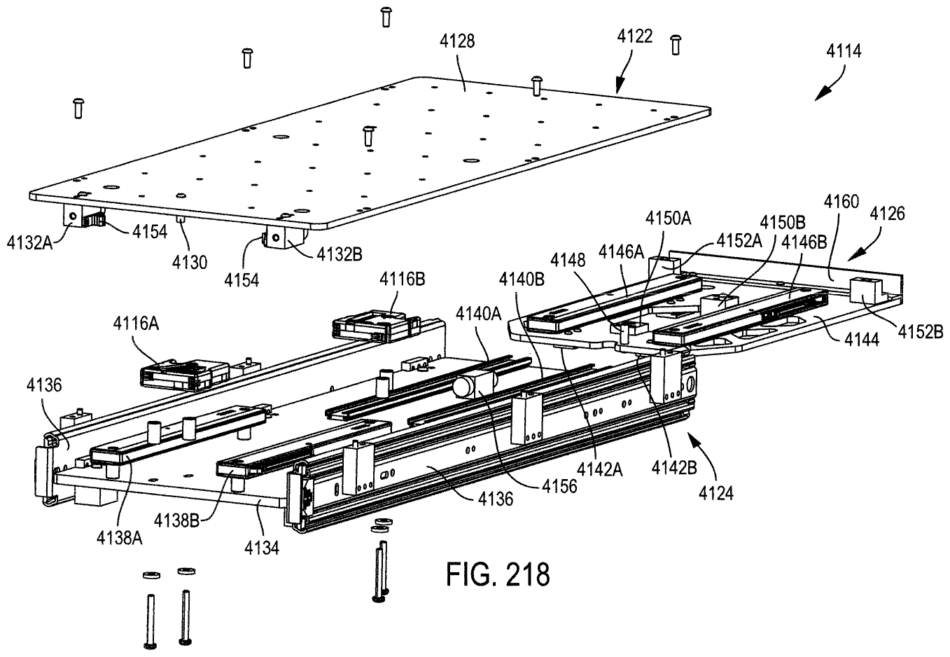

Referring now to FIGS. 215-217, illustrations of outfeed drawers 4102 in part of an example system 10 are shown. Components of the system 10 aside from the enclosure 12 and the outfeed drawers 4102 are hidden for sake of illustration. The top of the enclosure 12 has also been removed. As shown in FIG. 215, each outfeed drawer 4102 is depicted in a closed state. In the closed state, the interior of the enclosure 12 may be isolated from the exterior of the enclosure 12 by a seal formed between the enclosure 12 and the outfeed drawer 4102. One or more sealing member on the enclosure 12 and/or the exterior panel 4104 of each drawer 4102 may be included to assist in forming a seal when a drawer 4102 is in a closed state. Lock assemblies 4116A, B (see, e.g., FIG. 218) for each outfeed drawer 4102 may be in a displacement prohibiting state as a default.

Referring now primarily to FIG. 216, upon presentation and verification of a credential for a first access tier, the control system 15 may command actuation of a first lock assembly 4116A (see, e.g., FIG. 218) of a drawer assembly 4102 to a displacement permitting state. The outfeed drawer 4102 may be opened to a first stop position with the first lock assembly 4116A in a displacement permitting state. In the first stop position, the entire first receptacle array 4106 may be displaced out of the enclosure 12. The second receptacle array 4108 may remain within the enclosure 12. As mentioned above, the second receptacle array 4108 may be raised with respect to the first. The transition wall 4118 between the first and second receptacle array 4108 may be substantially even with the exterior wall of the enclosure 12 when the outfeed drawer 4102 is in the first stop position. Thus, the transition wall 4118 may block a user from reaching the second receptacle array 4108 and retrieving any of its contents. Opening of an outfeed drawer 4102 may be manual or alternatively may be under power of one or more electromechanical actuator.

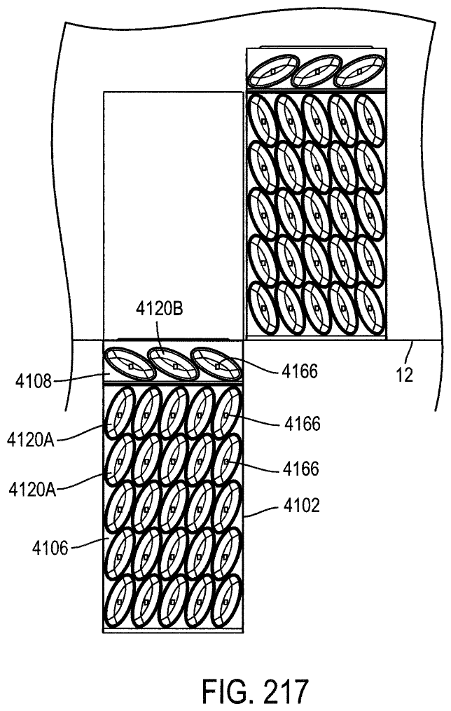

Referring now primarily to FIG. 217, upon presentation and verification of a credential for a second access tier, the control system 15 may command actuation of all lock assemblies 4116A, B (see, e.g., FIG. 218) of a drawer assembly 4102 to a displacement permitting state. The outfeed drawer 4102 may be opened to a second stop position with all lock assemblies 4116A, B in displacement permitting states. In the second stop position, the entirety of each of the first and second receptacle array 4106, 4108 may be displaced out of the enclosure 12.

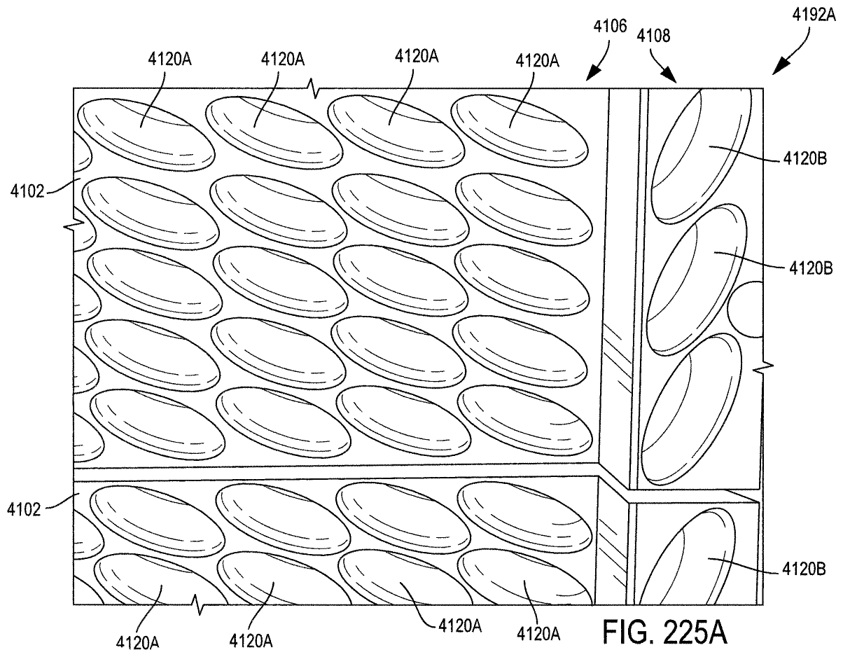

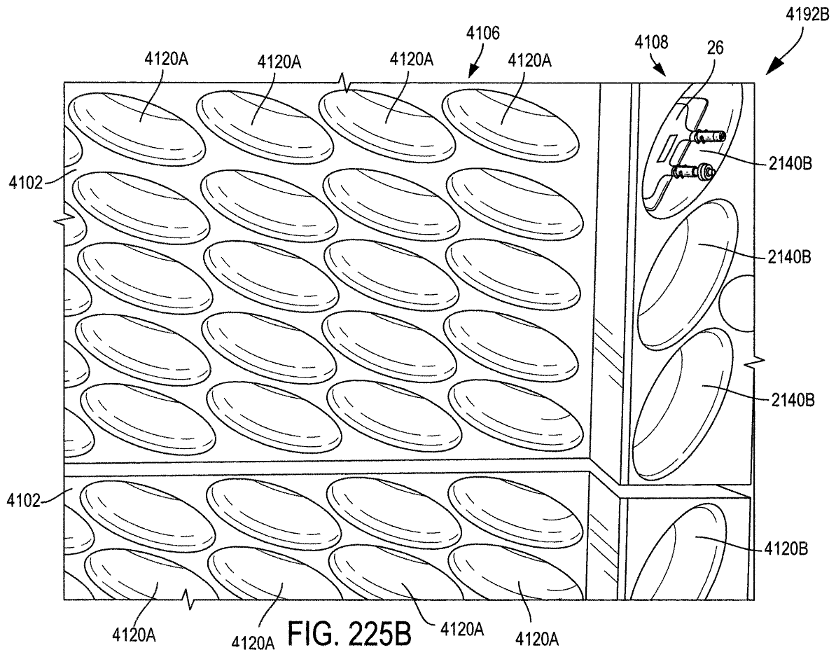

As shown in FIG. 217, one or more of the receptacles 4120A, B may be associated with a receptacle sensor 4166. The receptacle sensor 4166 may output a data signal indicative of whether a bag 26 is present in the respective receptacle 4120A, B. Any suitable sensor may be used. In certain examples, reflective type proximity sensors, beam break sensors, ultrasonic proximity sensors, button type sensors, weight based sensors, etc. may be used. In alternative embodiments, a gantry assembly 4200 (see, e.g., FIG. 221) may include one or more imager 4180 (see, e.g. FIG. 224) which may serve as a receptacle sensor 4166 for each receptacle 4120A, B. One or more imager 4180 may also or alternatively be mounted within the enclosure 12 at a fixed location providing a field of view of the receptacles 4120A, B. The imager(s) 4180 may collect image data of the outfeed drawers 4102 for analysis by the control system 15. The control system 15 may analyze image data from the imager(s) 4180 to identify which receptacles 4120A, B have been filled with a bag 26 (further described in relation to FIGS. 224-225B).

Where multiple outfeed drawers 4102 are included, the control system 15 may generate a prompt (e.g. via a GUI 6100, see, e.g., FIG. 1) for a user to indicate which outfeed drawer 4102 they desire to access. The prompt may differ depending on the type of credential presented or possessed by the user. Drawers 4102 which are empty may not be made available for selection. The control system 15 may command actuation of a first lock assembly 4116A, B of the selected outfeed drawer 4102 upon receipt of a selection input via a user interface of the system 10. Actuation of a second lock assembly 4116A, B of the selected outfeed drawer 4102 may also be commanded by the control system 15 in the event of an appropriate user input from a user with the requisite credential.

Referring now to FIG. 218, an exploded perspective view of an example drawer slide assembly 4114 is depicted. A drawer slide assembly 4114 may include a carriage 4122, a base assembly 4124, and a telescoping assembly 4126. The carriage assembly 4114 may include a mounting body 4128 to which an outfeed drawer 4102 may be coupled. A carriage striker 4130 may be included at one end region of the mounting body 4128 and may be flanked on each side by a carriage bumper 4132A, B. Bumpers 4132A, B may include at least one rubber contact. The outfeed drawer 4102 may mount to the side of the mounting body 4128 opposite the carriage striker 4130 and bumpers 4132A, B.

The base assembly 4124 may include a base body 4134. Drawer slides 4136 may be coupled to opposing sides of the base body 4134. When assembled, the carriage 4122 may mount to the displaceable portion of each drawer slide 4136. A set of displacement dampers 4138A, B and a set of guides 4140A, B may be coupled to the base body 4134. Two lock assemblies 4116A, B may also be coupled to the base body 4134. A bumper 4156 for the telescoping assembly 4126 may also be included on the base body 4134.

The telescoping assembly 4126 may include a main body 4144. A set of bearings 4142A, B which may ride along the guides 4140A, B of the base assembly 4124, may be attached to the main body 4144. A set of displacement dampers 4146A, B may be coupled to the opposing side of the main body 4144. A telescoping assembly striker 4148 may be coupled to an end region of the main body 4144. Additionally, the telescoping assembly 4126 may include a first and second set of stops 4150A, B, 4152A, B.

Referring now also to FIGS. 219A-C, a number of top plan views of an example drawer slide assembly 4114 are depicted. The example drawer slide 4114 is in a closed position in FIG. 219A and the mounting body 4128 of the carriage 4122 has been hidden. The exemplary drawer slide 4114 is shown at a first stop position in FIG. 219B and in a second stop position in FIG. 219C. In the closed position, the carriage striker 4130 and telescoping assembly striker 4148 may be in engaged states with respective lock assemblies 4116A, B. This may prohibit displacement of the carriage 4122 and opening of the outfeed drawer 4102.

With the first lock assembly 4116A commanded to an open state, the carriage striker 4130 may be released allowing displacement of the carriage 4122 and outfeed drawer 4102 coupled thereto to the first stop position. A trigger projection 4154 (see, e.g., FIG. 218) on each carriage bumper 4132A, B may activate the displacement dampers 4146A, B of the telescoping assembly 4126 as the carriage 4122 reaches an activation position near the first stop position. The displacement dampers 4146A, B may slow the travel of the carriage 4122 before the carriage 4122 reaches the first stop position. The displacement dampers 4146A, B may be self-close type drawer dampers and in such embodiments may automatically displace the carriage 4122 to the first stop position after the activation position is reached. The carriage bumpers 4132A, B may collide with the second set of stops 4152A, B of the telescoping assembly 4126 inhibiting further movement of the carriage 4122. As the telescoping assembly striker 4148 is engaged by the second lock assembly 4116B, the telescoping assembly 4126 may be inhibited from displacing and the outfeed drawer 4102 may be prevented from displacing to the second stop position. Thus, bags 26 cleared for use may be removed from the system 10 and distributed while rejected bags 26 may remain inaccessible.

In the event that the second lock assembly 4116B is actuated to a disengaged state, the telescoping assembly striker 4148 may be free to displace. The telescoping assembly 4126 may displace along the guides 4140A, B of the base assembly 4124 until the stop 4150A of the telescoping assembly 4126 contacts the bumper 4156 of the base assembly 4124. Though not shown, additional damper arrangements may be included to ensure that the telescoping assembly 4126 gently displaces to the end of its displacement range as the outfeed drawer 4102 reaches the second stop position. In certain examples, the second lock assembly 4116B may not be actuated to a disengaged state by the control system 15 until all bags 26 which have been cleared for use have been removed from that drawer 4102. In some embodiments, it may be required that the drawer 4102 is closed after removal of bags 26 cleared for use before the control system 15 will command actuation of the second lock assembly 4116B. The controls system 15 may verify all cleared bags 26 have been removed via the output signal(s) for any receptacle sensors 4166 or image analysis from data captured by an imager 4180.

Once any rejected bags 26 in the second receptacle array 4108 have been removed, the outfeed drawer 4102 may be displaced toward the closed state. The telescoping assembly 4126 may displace along the guides 4140 of the base assembly 4124 until the stop 4150B of the telescoping assembly 4126 contacts the bumper 4156 of the base assembly 4124. The trigger projections 4152 of the carriage 4122 may remain engaged with the displacement dampers 4146A, B of the telescoping assembly 4126 inhibiting displacement of the carriage 4122 relative to the telescoping assembly 4126. Once the stop 4150B collides with the bumper 4156, further force on the outfeed drawer 4102 may drive the trigger projections 4152 out of engagement with the displacement dampers 4146A, B displacing the carriage 4122 along the drawer slides 4136. As the carriage 4122 is displaced toward the closed position, the trigger projection 4154 (see, e.g., FIG. 218) on each carriage bumper 4132A, B may activate a respective carriage damper 4138A, B of the base assembly 4124. This may ensure that the drawer slide assembly 4114 does not slam into its closed position end stop. The carriage dampers 4138A, B may be of the self-closing variety and may help ensure that the carriage 4122 fully displaces to a repeatable closed position. A spring force may, for example, be exerted by the carriage dampers 4138A, B when the trigger projections 4154 are engaged. This may drive the carriage 4122 to the closed position. Additionally, if an outfeed drawer 4102 is released after a slight displacement in the opening direction, the carriage dampers 4138A, B may automatically return the outfeed drawer 4102 to the closed state. The telescoping assembly 4126 may include an end plate 4160 (see, e.g., FIG. 218). The outfeed drawer 4102 may include a projection, lip, etc. which contacts the end plate 4160 when the carriage 4122 is retracted to the closed position. This may ensure that the telescoping assembly 4126 is in a fully retracted state when the outfeed drawer 4102 is closed. This may also ensure that that the telescoping assembly striker 4148 will be engaged by the second lock assembly 4116B when the second lock assembly 4116B is in a locked state.

Still referring to FIGS. 219A-C, the outfeed assembly 4100 may include one or more position sensor 4162A, B for each outfeed drawer 4102. The control system 15 may monitor data from the position sensor 4162A, B to determine the position of the respective outfeed drawer 4102. A first position sensor 4162A may monitor the position of the carriage 4122. The signal output from the first position sensor 4162A may vary depending on whether the carriage 4122 is in a closed position or is at least partially open. The signal from a second position sensor 4162B may vary depending on whether the telescoping assembly 4126 is in its fully retracted position or has displaced from this position. The carriage 4122 and telescoping assembly 4126 may, for example, include magnetic bodies and the sensors 4162A, B may be Hall effect sensors. Microswitches which are actuated by projections on the carriage 4122 and telescoping assembly 4126 may be included. In some examples, a linear potentiometer may monitor the position of the carriage 4122 which may bear a wiper. Thus, the control system 15 may be able to continuously monitor the position of an outfeed drawer 4102 throughout its displacement range. These are merely examples, and any suitable sensors may be used.

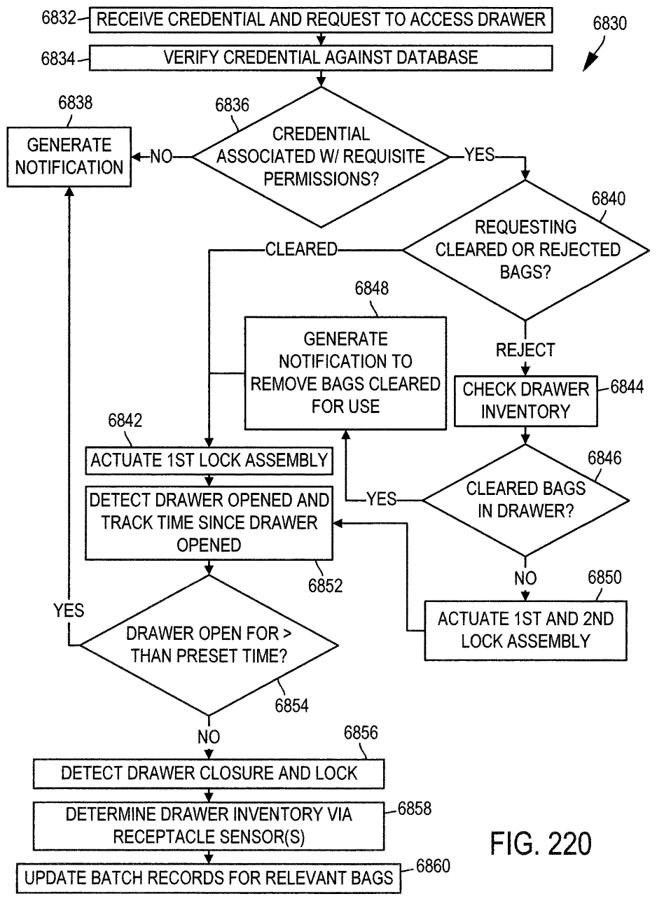

Referring now to FIG. 220, a flowchart 6830 is shown detailing a number of exemplary actions which may be executed to distribute bags 26 from a drawer 4102 of an outfeed assembly 4100. As shown, the control system 15 may receive a request to access a drawer and credential information for the requesting user in block 6832. The request may be input via a user interface 6100 (see, e.g., FIG. 1). The access request may specify a drawer 4102 and the type of contents the user would like to remove. The control system 15 may modify the options available for the access request depending on which drawers 4102 and/or receptacles 4120A, B are populated with bags 26. The credential may be supplied via an ID badge (e.g. RFID, NFC, barcode or data matrix on the ID, etc.), biometric, user name and password input, etc. In block 6834, the credential may be verified against a database. If, in block 6836, the credential is not associated with the requisite permissions in the database, a notification (e.g. display on the user interface 6100) may be generated by the control system 15. The request may be denied and no lock assemblies 4116A, B in the outfeed assembly 4100 may be disengaged. Where the control system 15 is in possession of the user credential before a request is submitted, the control system 15 may modify options available for the request depending on the permissions associated with the credential.

If, in block 6836, the credential is determined to have appropriate permissions, the control system 15 may behave differently depending on the type of contents the user is requesting access to. If, in block 6840, the user is requesting bags 26 which have been cleared for use, the control system 15 may disengage a first lock assembly 4116A of the drawer 4102 in block 6842. If, in block 6844, the user is requesting to remove rejected bags 26, the control system 15 may check the drawer inventory in block 6844. The drawer inventory may be determined by an analysis of data from one or more receptacle sensor 4166. Alternatively, a table of receptacles 4120A, B filled by the gantry assembly 4200 may be checked. If, in block 6846, bags 26 cleared for use remain in the drawer 4102, a notification may be generated in block 6848. The notification may be a display screen on a user interface 6100 (see, e.g., FIG. 1) to remove the cleared bags 26. The first lock assembly 4116A may be actuated in block 6842 to allow removal of the cleared bags 26. In some examples, the user may be required to reinitiate a request for access before the first lock assembly 4116A is disengaged by the control system 15. If, in block 6846, there are no cleared bags 26 in the drawer 4102, the control system 15 may disengage the first and second lock assemblies 4116A, B in block 6850.

Whether proceeding from block 6842 or block 6850, the control system 15 may detect opening of the drawer 4102 and track the time that the drawer 4102 is opened in block 6852. This may be accomplished by monitoring the output signals of one or more drawer position sensor 4162A, B (see, e.g., FIG. 219C) associated with the drawer 4102. If, in block 6855, the drawer 4102 has been in an open state for more than a preset period of time (e.g. 5 minutes), a notification may be generated in block 6838. The notification may include a display screen on the user interface 6100 drawing attention to the open drawer 4102 and/or an audio output (e.g. beep, series of tones, speech message). The period of time may differ depending on which lock assemblies 4116A, B are in a disengaged state. The period of time may be shorter if the lock assembly 4116A, B allowing access to rejected bags 26 is in a disengaged state. In some embodiments, the user may communicate (e.g. via a user interface 6100 input) that all bags 26 have been collected. The control system 15 may initiate a drawer closure timer after receiving input from the user that all bags 26 have been collected. A notification may be generated indicating the drawer closure timer has elapsed similarly to the notification described above in relation to block 6838 in the event closure of the drawer 4102 is not detected.

The control system 15 may detect closure of the drawer 4102 (e.g. via position sensor 4162A, B of FIG. 219C) in block 6856. The control system 15 may then determine inventory for the drawer 4102 in block 6858. This may be determined via the output from one or more drawer receptacle sensor 4166. Optionally, the user may be required to remove all bags 26 from the accessed portion of the drawer 4102 when a drawer 4102 is accessed. In the event that the inventory determination detects bags 26 in the drawer 4102, a notification may be generated in block 6838. The notification may include an audible output and a display screen on the user interface 6100 (see, e.g., FIG. 1) for example.

In block 6860, batch records for any relevant bags 26 (those in or removed from the drawer 4102) may be updated. Any logged information relating to removal of the bags 26 from the outfeed assembly 4100 may be written to the batch record for any bags 26 in an opened drawer 4102. For example, the control system 15 may document the user ID presented with the request to open a drawer 4102. The period of time the drawer 4102 was open may be documented. Additionally, where one or more receptacle sensor 4166 is included, the control system 15 may monitor data from the receptacle sensor(s) 4166 and indicate the time each particular bag 26 is confirmed to have been removed from the drawer 4102 in the respective bag's 26 batch record.

Referring now to FIG. 221-222, illustrations of outfeed drawers 4102 in part of an example system 10 are shown. For sake of illustration, certain components within the enclosure 12 are simplified or hidden. A gantry assembly 4200 may be included in an outfeed chamber 4202 of the enclosure 4200. The gantry assembly 4200 may collect bags 26 from the transfer chamber 3506 and displace the bags 26 throughout the outfeed chamber 4202. As described elsewhere herein, bags 26 may be mixed, subjected to particulate inspection, and labeled within the outfeed chamber 4202. The gantry assembly 4202 may shuttle bags 26 between stations for these purposes. The gantry assembly 4200 may include a gripper assembly 4204 which may be actuated open and closed around ports 392 of bags 26. With the ports 392 of a bag 26 held by the gripper assembly 4204, the gantry assembly 4200 may be actuated to displace to a coordinate which corresponds to a location within the outfeed chamber 4202. An X, Y coordinate may for example be specified and the control system 15 may command the gantry assembly 4200 to displace until it is determined that the specified coordinate has been reached. The gripper assembly 4204 may also be attached to a rotary actuator and may be displaceable to a specified rotational position.

The gantry assembly 4200 may displace bags 26 into alignment with a desired receptacle 4120A, B of an outfeed drawer 4102. The gripper assembly 4204 may then be commanded to an open state. The bag 26 then will drop into the targeted receptacle 4120A, B. The opening of the receptacle 4120A, B may be tapered or angled to help guide the bag 26 into the receptacle 4120A, B. In other embodiments, the gantry assembly 4200 may include a Z-axis displacement actuator and stage. In such embodiments, the gripper assembly 4204 may be displaced toward the target receptacle 4120A, B and the bag 26 may be released after being placed or at least partially introduced into the target receptacle 4120A, B. The target receptacle 4120A, B may be selected based on the bag's 26 conformity to one or more quality criteria (further described in relation to, e.g., Table 3 below). In the event that the bag 26 passes all requisite quality criteria, the target receptacle 4120A, B may be a receptacle 4120A of the first receptacle array 4106 (see FIG. 221). In the event the bag 26 fails at least one quality criteria, the target receptacle 4120A, B may be a receptacle 4120B in the second receptacle array 4108 (see FIG. 222). The control system 15 of the system 10 may store a record of which receptacles 4120A, B have already been populated with a bag 26. Alternatively or additionally, each receptacle 4120A, B may be associated with a receptacle sensor 4166 (see, e.g., FIG. 217) and the control system 15 may determine which receptacles 4120A, B are unfilled based on the signals from the receptacle sensors 4166. As further described elsewhere herein, outfeed drawer 4102 inventory may also be tracked via one or more imager 4180 (see, e.g., FIG. 224). The target receptacle 4120A, B may always be an empty receptacle 4120A, B. The control system 15 may also generate a notification in the event that one of the receptacle arrays 4106, 4108 for an outfeed drawer 4102 is filled.

In some embodiments, the control system 15 may not prohibit further operation of the system 10 if a second receptacle array 4108 is filled and another second receptacle array 4108 has unpopulated receptacles 4120B. In the event that all receptacles 4120B in the outfeed chamber 4202 have been filled, the control system 15 may prevent further bags 26 from being processed by the system 10. The control system 15 may generate a notification that a properly credentialed user must empty the second receptacle arrays 4108 of the outfeed assembly 4100 and dispose of the bags 26 therein. The control system 15 may prohibit further operation of the system 10 in the event that all first receptacle arrays 4106 in the outfeed assembly 4100 have been filled. A notification for a properly credentialed user to empty the first receptacle arrays 4106 may be generated.

The control system 15 may check the signals from any receptacle sensors 4166 (or take and analyze one or more image from any imagers 4180) to ensure bags 26 have been removed after an outfeed drawer 4102 has opened. In the event that bags 26 remain in the first receptacle array 4106, but at least one receptacle 4120A is unpopulated, the control system 15 may command production activities to resume. If bags 26 remain in a second receptacle array 4108, however, the control system 15 may generate a notification that the bags 26 must be removed. In alternative embodiments, the control system 15 may command production activities resume so long as at least one receptacle 4120A, B from each array 4106, 4108 type is determined to be unfilled.

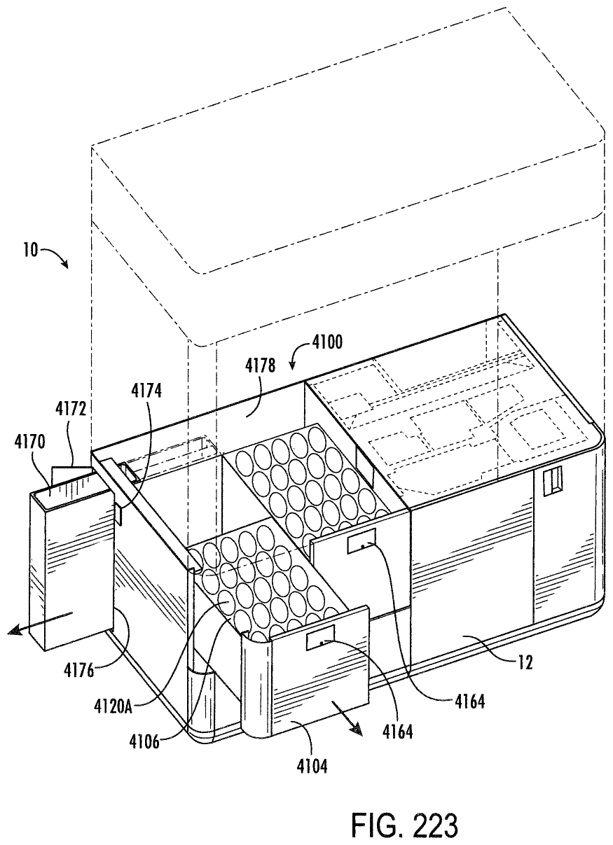

Referring now to FIG. 223, an alternative example outfeed assembly 4100 is depicted in an example system 10. The enclosure 12 of the system 10 is partially shown in phantom and some components of the system 10 have been hidden or simplified for sake of illustration. As shown, the outfeed assembly 4100 may include a plurality of outfeed drawers 4102 which include first arrays of receptacles 4106. The outfeed drawers 4102 may be coupled to drawer slide assemblies 4114 and may be opened to retrieve bags 26 out of the first receptacle arrays 4106. The outfeed drawers 4114 may be simplified variants of those shown in FIG. 218-219C. The outfeed drawers 4114 may include a single lock assembly 4116A and may omit a telescoping portion. Acceptable bags 26 may be placed in the receptacles 4120A of the outfeed drawers 4102.

The outfeed assembly 4100 may include a discard receptacle 4170 which may be segregated from the outfeed drawers 4102. Any bags 26 which do not meet all required quality criteria may be transported to the discard receptacle 4170 by the gantry assembly 4200 and deposited therein. The discard receptacle 4170 may be displaced at least partially out of the enclosure 12 via a discard access 4172. The discard access 4172 may be a door or hatch which may be locked absent presentation and verification of a specialized credential and user input to the system 10 requesting the discard access 4172 be unlocked. A discard access state sensor 4174 may be included and may output a signal which varies depending on whether the discard access 4172 is opened or closed. When the discard access 4172 is opened, the discard receptacle 4170 may be drawn out of an aperture 4176 of the enclosure 12 and any contents may be removed and disposed of.

Referring now to FIG. 224, a block diagram of a portion of an example outfeed assembly 4100 is depicted. The outfeed assembly 4100 may include one or more outfeed drawers 4102 (only one is shown for sake of simplicity). The example outfeed drawers 4102 are of the variety described in relation to FIG. 214 and include first receptacles 4120A and second receptacles 4120B belonging to respective first and second receptacle arrays 4106, 4108. Bags 26 are depicted in each receptacle 4120A, B. One or more outfeed inventory imagers 4180 having a line of sight into the receptacles 4120A, B may be included. In the example, two imagers 4180 are shown. Imagers 4180 may be positioned such that the fields of view 4190 of the imagers 4180 collectively capture images of all receptacles 4120A, B in all outfeed drawers 4102. As shown in FIG. 224, some receptacles 4120A, B may be in the field of view 4190 of a plurality of imagers 4180. One or more light source 4188 may also be included in example outfeed assemblies 4100 and may be powered to illuminate the outfeed drawers 4102 during imaging.

Where the receptacles 4120A, B are oval or elliptical in cross-sectional shape, an imager 4180 may be mounted above the second array 4108 of receptacles 4120B for each respective drawer 4102. In examples with two drawers 4102 having pockets with mirrored orientations (see, e.g., FIG. 215), the number of imagers 4180 may equal the number of drawers 4102. Each imager 4180 may be positioned to have a viewing axis which is aligned with or generally parallel (e.g. within 35°) to planes including the major axes of the receptacles 4120A disposed opposite the second array 4108 in the adjacent drawer 4102. This may assist in maximizing the number of receptacles 4120 each imager 4180 has a sight line into while limiting the number of imagers 4180 required.

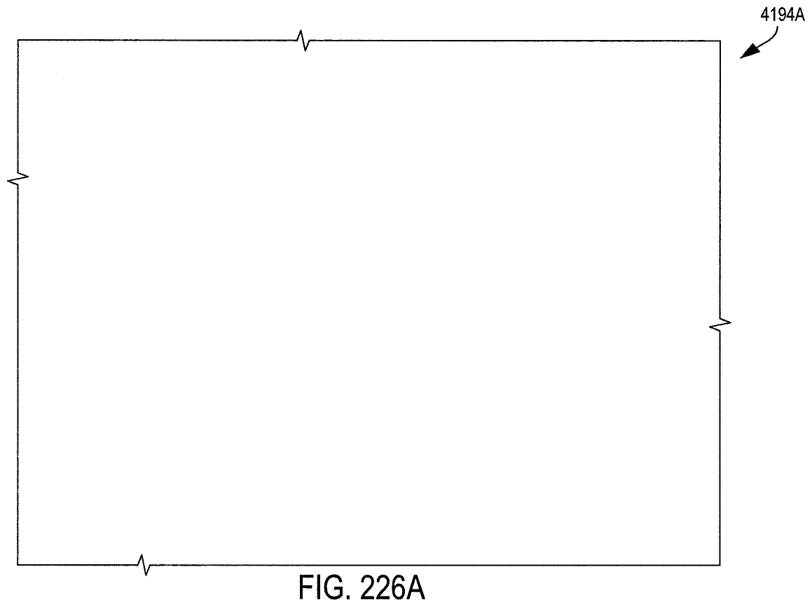

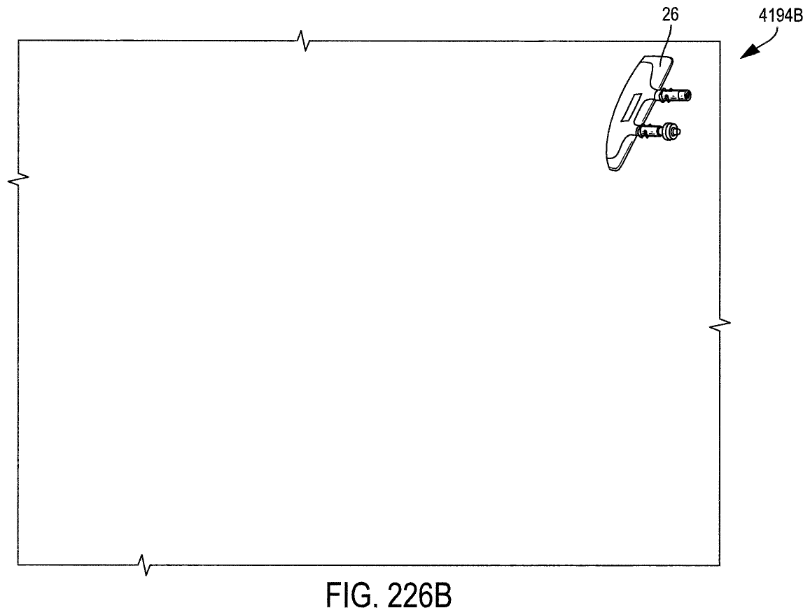

Referring now to FIGS. 225A-B, representational illustrations of images 4192A, B from one outfeed inventory imager 4180 are depicted. In FIG. 225A, no receptacles 4120A, B are populated with a bag 26. In FIG. 225B, one access controlled receptacle 4120B is populated with a bag 26. To verify the inventory of bags 26 in an outfeed drawer 4102, the control system 15 may analyze the images from each of the outfeed inventory imagers 4180. In some examples, the images may be compared to reference images for each camera. The reference images may be images of the outfeed drawers 4102 in a closed state when the outfeed drawers 4102 are known to be empty. These reference images may be taken by, for example, a service or setup technician. In certain implements, the control system 15 may conduct a background subtraction using the image captured from each outfeed inventory imager 4180 and the respective reference image for that imager 4180. In various embodiments, the receptacles 4120A, B may be formed of a black material or of material having a fully saturated color (e.g. red, blue, green). This may facilitate use of monochrome outfeed inventory imagers 4180. In a scenario where all receptacles 4120A, B are empty (see, e.g. FIG. 225A), the resultant foreground segmented image may be substantially blank as shown in FIG. 226A. Where bags 26 are present in receptacles 4120A, B (see, e.g., FIG. 225B), the foreground segmented image may reflect the presence of such bags 26. This is shown in the illustrative example foreground segmented image 4194B in FIG. 226B. The foreground segmented images 4194A, B in FIGS. 226A-B are simplified, illustrative representations and not exact reproductions of actual images.

The foreground segmented images may be analyzed for regions of pixels consistent with bags 26. In some embodiments, filtering (e.g. denoising) may be conducted to the raw images from the outfeed inventory imagers 4180 before background subtraction. The images from the outfeed inventory imagers 4180 may also be registered or aligned with the reference images in some examples (the outfeed drawers 4102 may include one or more fiducial reference to assist in image alignment). The foreground segmented images may be subject to binary thresholding. Additionally, filtering may be performed to remove foreground pixels which are invalid. For example, a morphological transformation (kernel dilation) may be performed and then contours may be drawn around continuous regions of foreground pixels. Any continuous regions below a certain size may be excluded from further analysis. Contours in a predetermined certain size range may be checked to determine whether the constituent pixels of each contour fall within specific sets of pixel coordinates. These specific sets of pixel coordinates may correspond to the locations of receptacles 4120A, B within the raw images. In the event that a collection of pixels having a contour within a predetermined acceptable size range is present within one of the sets of coordinates, the control system 15 may determine that the receptacle 4120A, B corresponding to that set of coordinates is populated with a bag 26. The control system 15 may generate a fault and orchestrate display of a notification on the user interface 6100 of the system 10 in the event that a collection of pixels in the certain size range is in a position which does not correspond to receptacle 4120A, B. The control system 15 may also check for contours having a pixel area beyond the predetermined acceptable size range. The presence of any contours having an area in a predetermined unacceptable size range may result in the control system 15 generating a fault and orchestrating display of a notification on the user interface 6100. Such pixel collections may be representative of a bag 26 which has missed a receptacle 4120A, B and is laying atop the outfeed drawer 4102.

In some examples, the control system 15 may also analyze images to detect that a bag 26 was properly received by a target receptacle 4120A, B. An image from an appropriate outfeed inventory imager 4180 may be captured after a gantry assembly 4200 releases a bag 26. The image may be compared against a reference image as described above to ensure a collection of foreground pixels representative of a bag 26 is detected in a set of image coordinates corresponding to the target receptacle 4120A, B. If a collection of pixels is not present in the set of image coordinates corresponding to the target receptacle 4120A, B, the control system 15 may generate a fault and orchestrate display of a notification on the user interface 6100 of the system 10. Likewise, if image analysis results in a detection of an unexpected bag 26 in a receptacle 4120A, B a fault and notification may be generated by the control system 15.

A list or table of populated receptacles 4120A, B may be updated by the control system 15 as bags 26 are added and removed from the receptacles 4120A, B. The specific receptacle 4120A, B in which a bag 26 is stored may be written to the batch record associated with that particular bag 26 by the control system 15. A total time the bag 26 was stored in the receptacle 4120A, B may also be tracked and documented in the respective bag 26 batch record. This time period may run from the time the bag 26 was deposited by the gantry assembly 4200 to the point the bag 26 was confirmed to have been removed from an outfeed assembly 4100. A user identity associated with the credential used to remove any bags 26 may also be documented in the batch record for each bag 26.

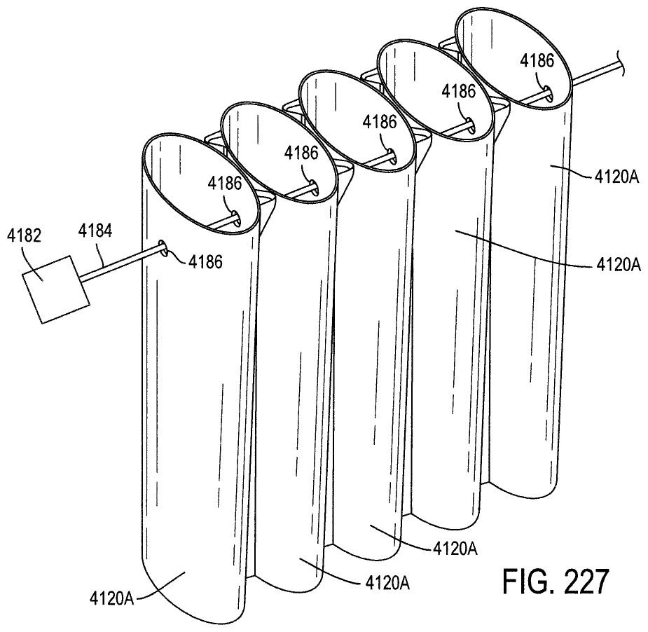

Referring now to FIG. 227, another example outfeed inventory sensor 4182 is depicted. As shown, only a single row of receptacles 4120A is depicted for sake of illustration. An outfeed inventory sensor 4182 of the type shown in FIG. 227 may be included for each row of receptacles 4120A, B in an outfeed drawer 4102. The example outfeed inventory sensor 4182 is depicted as a time of flight sensor. Each of the receptacles 4120A, B in a row may include a pair of apertures 4186. The apertures 4186 for receptacles 4120A, B within a given row may be aligned and substantially coaxial. The size of the apertures 4186 may be sufficient to allow passage of the beam 4184 from the time of flight type outfeed inventory sensor 4182. Though shown in a continuous row, the row of receptacles 4120A, B may span multiple distinct outfeed drawers 4102 in some implementations.

Still referring to FIG. 227, as the receptacles of an outfeed drawer 4102 are filled, the furthest receptacle 4120A, B from the outfeed inventory sensor 4182 in a row may be filled first. The second most distal receptacle 4120A, B from the sensor 4182 may be filled next. This may continue until the receptacle 4120A, B most proximate the sensor 4182 has been populated. As each receptacle 4120A, B is populated in this order, the distance measured by the time of flight type outfeed inventory sensor 4182 will decrease in a predictable manner. The control system 15 may monitor the output signal from each outfeed inventory sensor 4182 to ensure that the decrease in distance is indicative of a bag 26 being received by the target receptacle 4120A, B. In the event the measured distance does not change by more than a predetermined amount or the decrease is greater than a predefined amount, the control system 15 may generate a fault and orchestrate display of a notification on the user interface 6100 of the system 10. Regardless of the sensing arrangement, where a fault generated, the system 10 may require an appropriately credentialed user to service the outfeed assembly 4100 or remove bags 26 from the drawers 4102.

The results of any inventory determinations may be written to a log and stored in a database. Additionally, where it is verified that each bag 26 is deposited in its target receptacle 4120A, B this may be included in an inspection log 3590 (see, e.g., FIG. 179) for the bag 26. Supporting data (e.g. image data from outfeed inventory imagers 4180 or other outfeed inventory sensor 4182, 4166) may also be included with the inspection log 3590.

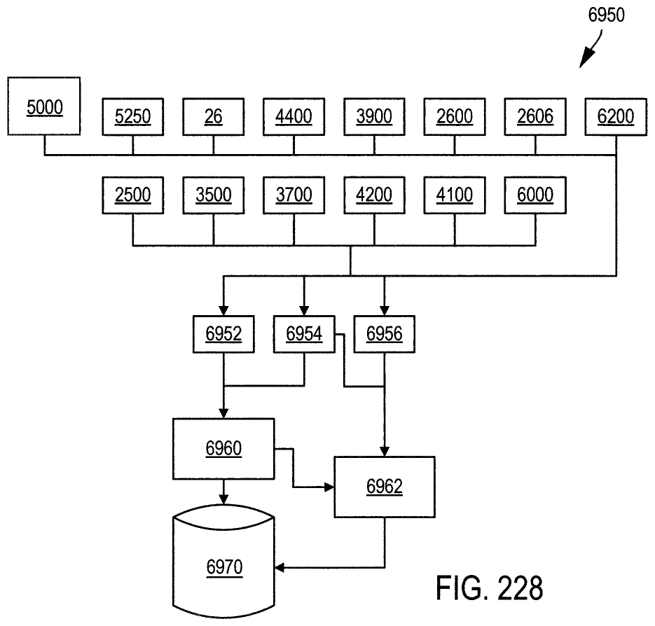

Referring now to FIG. 228, an exemplary data flow diagram 6950 is depicted. As bags 26 are produced by the system 10, the control system 15 may collect and update various data pertaining to that bag 26. As shown in FIG. 228, some of this data may support generation of a bag quality record 6960 as well as a batch record 6962 for each bag 26. The control system 15 may serialize (e.g. in JSON format or any other suitable notation) and communicate any bag records to a database 6970 for storage and remote access. As shown, the control system 15 may collect a variety of types of data from subsystems within the system 10. The example diagram in FIG. 228 depicts data relating to the dispenser 5000, docking assembly 5250, bag 26, singulating assembly 4400, fluid handling system 3900, fill station 2600, cartridge 2606, air handling assembly 6200, mix assisting assembly 2500, particulate inspection system 3500, marking assembly 3700, gantry assembly 4200, outfeed assembly 4100, and pneumatic distribution system 6000 being collected. Data types may include quality decision data 6952, odometry data 6954, and supporting data 6956. The quality decision data 6952, odometry data 6954, and supporting data 6956 described in the following paragraphs and tables is merely exemplary. In certain embodiments, not all of the example data may be included and/or additional data may be included.

The quality decision data 6952 may be data relating to requirements imposed on the production of a particular bag 26 in order for that bag 26 to be releasable for consumption. This quality decision data 6952 may include pass/fail indications for production actions the bag 26 is subjected to or that are performed on the bag 26. Measured values used for supporting the determination as to whether the bag 26 meets acceptance criteria may be included. The quality decision data 6952 may further include summary or statistical data (e.g. min, max, average, standard deviation, etc.) from sensors relevant to each imposed acceptance criterion. Any summary or statistical data may be collected over the entire period where bags 26 are being filled. Alternatively or additionally, summary data specific to a period of time most relevant to a particular bag 26 may be included. For example, summary or statistical data relating to environmental control may be provided over the time period where the bag 26 is exposed to the processing compartment 4300. The summary or statistical data may not be based on any information collected after the dispensing sharp 2604 has been withdrawn from a septum 393 for a given bag 26. Summary or statistical data from the fluid handling system 3900 may be provided for the period of time the bag 26 is being filled with fluid.

Quality decision data 6952 may also include pass/fail indications at various checkpoints in the production process for a bag 26. For example, the quality decision data 6952 may include a pass/fail indication for a checkpoint just prior to marking by a marking assembly 3700 and before determining a receptacle 4120A, B in the outfeed assembly 4100 to deposit the bag 26 into. The outcome of, for instance, the pre-marking checkpoint verification may inform the content of the marking made on the bag 26 (e.g. an expiration date or a reject marking). Other verification checkpoints may also be included. For example, the control system 15 may query a database 6970 when a bag 26 indicium 56 or dispenser 5000 indicium 5014 is read. Such verification checkpoints may be documented as a pass in the event that the database 6970 indicates the component is acceptable. These verification checkpoints may be indicated as a failure in the event that the database 6970 entry associated with a unique identifier included on the indicium 56, 5014 indicates a recall or other issue.

Various identifiers, temporal, and date information may also be captured as quality decision data 6952. The quality decision data 6952 may be a record of in-process actions on the bag 26 supporting a decision of whether the bag 26 is acceptable for use or should be rejected. Example quality decision data 6952 which may be tracked by example systems 10 described herein is provided in Table 3 below:

TABLE 3

| | | |
|---|---|---|
| Attribute Type | Attribute | Description |

General

| | | |
|---|---|---|
| Date | Date | Date Bag Quality Record 6960 was created or last |
| | | updated (dates may include e.g. year, month, day, hour, |
| | | minute, and second) |
| Identifier | Production Run ID | Unique identifier assigned to a specific order for a bag |
| | | 26 or bags 26 from the system 10 |

Dispenser Docking Assembly 5250

| | | |
|---|---|---|
| Identifier | Dispenser ID | Unique identifier assigned to specific dispenser 5000 |
| | | (may also serve a chain 2670 identifier) |
| Identifier | Dispenser Lot No. | Unique identifier assigned to specific production lot to |
| | | which an individual dispenser 5000 belongs |
| Date | Dispenser Expiry | Expiration date for dispenser 5000 |
| | Date | |
| Process | Dispenser | Start and stop time as well as pass/fail indication as to |
| Outcome | Inspection Record | whether indicium 5014 on dispenser 5000 could be |
| | | successfully read. |

Bag Information

| | | |
|---|---|---|
| Identifier | Bag ID | Unique identifier assigned to a specific bag 26 by the |
| | | system 10 as that bag 26 enters a system 10 |
| Identifier | Bag Index ID | Number indicating position of bag 26 in parent chain |
| | | 2670 |
| Identifier | Bag Production | Number indicating where bag 26 falls within a specific |
| | Run Index No. | production run (e.g. bag 14/50) |
| Identifier | Batch ID | Unique identifier assigned to a specific batch |
| Identifier | Batch API | Name of each active ingredient and measure of each |
| | | active ingredient per unit of measure of drug product |
| | | (may also be unique identifier for relevant master batch |
| | | record) |
| Identifier | Bag Label ID | Unique identifier assigned to a specific bag 26 and |
| | | collected from indicium 56 on bag 26 |
| Identifier | Bag Lot No. | Unique identifier assigned to specific production lot to |
| | | which an individual bag 26 belongs and collected from |
| | | indicium 56 on bag 26 |
| Process | Bag ID Outcome | Start and stop time as well as pass/fail indication as to |
| Outcome | | whether indicium 56 on bag 26 was successfully read. |
| Position | Bag Location | Current location of bag 26 (e.g. fill station 2600, mix |
| | | assisting assembly 2500, distributed for use, etc.) |

Bag Individualizing Assembly 4400

| | | |
|---|---|---|
| Bag Trait | Peak Torque | Peak torque reading during singulation of bag 26 from |
| | | chain 2670 |
| Bag Trait | Position of Peak | Position data for point at which peak torque during |
| | Torque | singulation of bag 26 from chain 2670 occurred |
| Statistical Data | Singulation Torque | Torque applied to drive displacement of splitter |
| | | assembly 4406 (e.g. as percentage of actuator's 4402 |
| | | max torque ) |
| Process | Singulation | Start and stop time as well as pass/fail indication as to |
| Outcome | Outcome | whether a bag 26 was successfully singulated |

Fluid Handling System 3900

| | | |
|---|---|---|
| Statistical Data | Inlet Conductivity | Inlet fluid conductivity indicated by inlet fluid |
| | | conductivity sensor 3906 |
| Statistical Data | Inlet Flow Rate | Flow rate indicated by output signal from input flow |
| | | sensor 3910 |
| Statistical Data | Inlet Pressure | Inlet pressure indicated by output signal of inlet pressure |
| | | sensor 3904 |
| Statistical Data | Inlet Temperature | Temperature indicated by output signal from inlet fluid |
| | | conductivity sensor 3906 |
| Statistical Data | Carbon Filter | Pressure indicated by output signal of post carbon |
| | Outlet Pressure | filtration pressure sensor 3922 |
| Statistical Data | Accumulator | Temperature indicated by output signal from |
| | Temperature | accumulator temperature sensor 3952 |
| Statistical Data | Accumulator | Sensed current from irradiator 3956 |
| | Outlet UV | |
| | Intensity | |
| Statistical Data | RO Pump Outlet | Pressure indicated by output signal of pump outlet |
| | Pressure | pressure sensor 3958 |
| Statistical Data | RO Pump Outlet | Pump output conductivity indicated by output signal |
| | Conductivity | from pump outlet conductivity sensor 3960 |
| Statistical Data | RO Pump Outlet | Temperature indicated by output signal from pump |
| | Temperature | outlet conductivity sensor 3960 |
| Statistical Data | RO Pump | ((Pressure indicated by pump outlet pressure sensor |
| | Transmembrane | 3958 + Pressure indicated by retentate pressure sensor |
| | Pressure | 3980)/2) − Pressure indicated by permeate pressure |
| | | sensor 3990 |
| Statistical Data | RO Retentate | Retentate conductivity indicated by output signal from |
| | Conductivity | retentate conductivity sensor 3984 |
| Statistical Data | RO Retentate Flow | Flow rate indicated by output signal from retentate flow |
| | Rate | sensor 3982 |
| Statistical Data | RO Retentate | Pressure indicated by output signal of retentate pressure |
| | Pressure | sensor 3980 |
| Statistical Data | RO Retentate | Temperature indicated by output signal from retentate |
| | Temperature | conductivity sensor 3984 |
| Statistical Data | RO Permeate | Permeate conductivity indicated by output signal from |
| | Conductivity | permeate conductivity sensor 3994 |
| Statistical Data | RO Permeate Flow | Flow rate indicated by output signal from permeate flow |
| | Rate | sensor 3992 (may also be utilized as total feed flow rate |
| | | to polishing unit 3988) |
| Statistical Data | RO Permeate | Pressure indicated by output signal of permeate pressure |
| | Pressure | sensor 3990 |
| Statistical Data | RO Permeate | Temperature indicated by output signal from permeate |
| | Temperature | conductivity sensor 3994 |
| Statistical Data | RO Rejection Rate | (1-(Conductivity indicated by permeate conductivity |
| | | sensor 3994/Conductivity indicated by pump outlet |
| | | conductivity sensor 3960))*100 |
| Statistical Data | Electrodeionization | Electrodeionization unit 3988 current |
| | Unit Current | |
| Statistical Data | Electrodeionization | Electrodeionization unit 3988 voltage |
| | Unit Voltage | |
| Statistical Data | Electrodeionization | Flow rate indicated by output signal from electrode |
| | Unit Electrode | stream flow sensor 3999 |
| | Flow Rate | |
| Statistical Data | Polished Fluid | Flow rate indicated by output signal from polished fluid |
| | Flow Rate | flow sensor 4019 |
| Statistical Data | Polished Fluid | Sensed current from irradiator 4010 |
| | Outlet UV | |
| | Intensity | |
| Statistical Data | UF Inlet Pressure | Pressure indicated by output signal from polished fluid |
| | | output temperature sensor 4024 |
| Statistical Data | UF Permeate | Pressure indicated by output signal from dispensing flow |
| | Pressure | path pressure sensor 4034 |
| Statistical Data | UF Permeate | Dispensing flow path conductivity indicated by |
| | Conductivity 1 | dispensing flow path conductivity sensor 4050A |
| Statistical Data | UF Permeate | Dispensing flow path conductivity indicated by |
| | Conductivity 2 | dispensing flow path conductivity sensor 4050B |
| Statistical Data | UF Permeate | Temperature indicated by output signal from dispensing |
| | Temperature 1 | flow path conductivity sensor 4050A |
| Statistical Data | UF Permeate | Temperature indicated by output signal from dispensing |
| | Temperature 2 | flow path conductivity sensor 4050B |
| Statistical Data | Total Organic | Total organic carbon ppb indicated by total organic |
| | Carbon | carbon sensor 4038 |
| Statistical Data | Drain Conductivity | Dispensing flow path drain conductivity indicated by |
| | | conductivity sensor 4040 of total organic carbon sensor |
| | | 4038 |
| Statistical Data | Drain Temperature | Temperature indicated by output signal from |
| | | temperature sensor 4042 of total organic carbon sensor |
| | | 4038 |
| Statistical Data | Dispense Flow | Flow rate indicated by output signal from dispensing |
| | Rate 1 | fluid flow path flow sensor 4052A |
| Statistical Data | Dispense Flow | Flow rate indicated by output signal from dispensing |
| | Rate 2 | fluid flow path flow sensor 4052B |
| Process | Fluid Production | Start and stop time as well as pass/fail indication as to |
| Outcome | Outcome | whether sensor data from fluid handling system indicates |
| | | conformity to any predefined respective thresholds |
| | | during time period relevant to a bag's 26 processing |

Fill Station 2600

| | | |
|---|---|---|
| Date | Date of | Fill timestamp (e.g. start time) for a specific bag 26 |
| | Manufacture | |
| Bag Trait | Dispensed Volume | Average of the dispensed volume indicated by from |
| | into Bag | dispensing flow path flow sensors 4052A, B |
| Bag Trait | Total Flow | Totalized flow into bag 26 based on data from |
| | Through | dispensing flow path flow sensor 4052A |
| | Dispensed Fluid | |
| | Flow Sensor 1 into | |
| | Bag | |
| Bag Trait | Total Flow | Totalized flow into bag 26 based on data from |
| | Through | dispensing flow path flow sensor 4052B |
| | Dispensed Fluid | |
| | Flow Sensor 2 into | |
| | Bag | |
| Bag Trait | Deviation between | Measure of deviation between total flow into bag 26 |
| | Dispensed Flow | indicated by dispensing flow path flow sensors 4052A, |
| | Sensors | B (e.g. delta between total volumes dispensed as |
| | | measured by each sensor 4052A, B) |
| Bag Trait | Total Mass | Calculated value based on average of totalized flow into |
| | Dispensed into | bag 26 from dispensing flow path flow sensors 4052A, |
| | Bag | B and temperature (e.g. from cartridge input temperature |
| | | sensor 4054A) |
| Bag Trait | Fluid Flow Sensor | Calculated value based on totalized flow into bag 26 |
| | 1 Total Dispensed | from dispensing flow path flow sensor 4052A and |
| | Mass into Bag | temperature (e.g. from cartridge input temperature |
| | | sensor 4054A) |
| Bag Trait | Fluid Flow Sensor | Calculated value based on totalized flow into bag 26 |
| | 2 Total Dispensed | from dispensing flow path flow sensor 4052A and |
| | Mass into Bag | temperature (e.g. from cartridge input temperature |
| | | sensor 4054A) |
| Bag Trait | Fill Cartridge Flow | Average of totalized flow into bag 26 from dispensing |
| | Coefficient | flow path flow sensors 4052A, B * LPM_TO_GPM * |
| | | sqrt(Specific Gravity/(pressure indicated by cartridge |
| | | input pressure sensor 4056-pressure from head height |
| | | at dispensing sharp 2064)) |
| Process | Fill Outcome | Start and stop time as well as pass/fail indication as to |
| Outcome | | whether bag 26 was filled in accordance with predefined |
| | | acceptability criteria |
| Bag Trait | Antimicrobial | On time for irradiation assembly 2608 for specific bag |
| | Light Exposure | 26 prior to spiking of septum 393 |
| | Duration | |
| Process | Septum Irradiation | Start and stop time as well as pass/fail indication as to |
| Outcome | | whether irradiation assembly 2608 powered on as |
| | | expected |

Air Handling System 6200

| | | |
|---|---|---|
| Statistical Data | Intake Humidity | Humidity as indicated by output signal from intake |
| | | humidity sensor 6300 |
| Statistical Data | Intake Particulate | Particulate reading(s) indicated by output signal(s) from |
| | | post pre-filter particulate sensor 6304 |
| Statistical Data | Intake Pressure | Pressure indicated by output signal of intake pressure |
| | | sensor 6302 |
| Statistical Data | Pre-filter Pressure | Pressure drop across pre-filter 6204 calculated from |
| | Drop | pressured indicated by intake pressure sensor 6302 and |
| | | ambient pressure |
| Statistical Data | Air Temperature | Temperature as indicated by output signal from blower |
| | | temperature sensor 6310 |
| Statistical Data | Blower Airflow | Flow rate as indicated by output signal of blower flow |
| | | rate sensor 6308 |
| Statistical Data | Relative Humidity | Humidity as indicated by output signal from blower |
| | | humidity sensor 6306 |
| Statistical Data | ULPA Filter | Pressure drop across filter calculated from pressures |
| | Pressure Drop | indicated by filtration chamber pressure sensor 6314 and |
| | | post ULPA filter pressure sensor 6316 |
| Statistical Data | Fill Zone Air | Particulate reading(s) indicated by output signal(s) from |
| | Particulate | post ULPA filter particulate sensor 6318 |
| Statistical Data | Fill Zone Gauge | Pressure indicated by output signal from post ULPA |
| | Pressure | filter pressure sensor 6316 |
| Statistical Data | Fill Zone Humidity | Humidity indicated by output signal from post ULPA |
| | | humidity sensor 6319 |
| Statistical Data | Fill Zone to | Pressure difference calculated from pressures indicated |
| | Surrounding Zone | by output signals of post ULPA filter pressure sensor |
| | Pressure Delta | 6316 and processing chamber pressure sensor 6320 |
| Statistical Data | Particulate in Zone | Particulate reading(s) indicated by output signal(s) from |
| | Surrounding Fill | processing chamber particulate sensor 6322 |
| | Zone | |
| Statistical Data | Processing | Pressure difference calculated from pressures indicated |
| | Chamber to | by output signals of processing chamber pressure sensor |
| | Transfer Chamber | 6320 and transfer chamber pressure sensor 6330 |
| | Pressure Delta | |
| Statistical Data | Transfer Chamber | Particulate reading(s) indicated by output signal(s) from |
| | Particulate | transfer chamber particulate sensor 6332 |
| Process | Environmental | Start and stop time as well as pass/fail indication as to |
| Outcome | Control Outcome | whether sensor data from air handling system indicates |
| | | conformity to any predefined respective thresholds |
| | | during time period relevant to a bag's 26 processing |

Mix Assisting Assembly 2500

| | | |
|---|---|---|
| Bag Trait | Motion Profile | Measure of displacement error for each set of position |
| | Deviation | pairs in a motion profile |
| Process | Mix Outcome | Start and stop time as well as pass/fail indication as to |
| Outcome | | whether bag 26 was mixed within acceptable deviations |
| | | from predefined motion profile. |

Particulate Inspection System 3500

| | | |
|---|---|---|
| Bag Trait | Total Number of | Total number of tracks for detected regions of interest |
| | Bubbles | classified as belonging to bubbles |
| Bag Trait | Total number of | Total number of tracks for detected regions of interest |
| | Particles | classified as belonging to particulates |
| Process | Particulate | Start and stop time as well as pass/fail indication. |
| Outcome | Inspection | Indicated as a failure if any contents are classified as |
| | Outcome | something other than a bubble. |

Marking Assembly 3700

| | | |
|---|---|---|
| Process | Pre-Marking | Indication as to whether a given bag has been |
| Outcome | Disposition | determined to be acceptable or rejected prior to marking |
| Date | Bag Expiry Date | Expiration Date for the bag 26 (e.g. fill start/end time + |
| | | predetermined value) |
| Bag Trait | Marking String | Expiration date (or reject mark) commanded to be |
| | | marked on bag by marking assembly 3700 |
| Process | Emboss Outcome | Start and stop time as well as pass/fail indication as to |
| Outcome | | whether marking of bag 26 was successful |
| Bag Trait | Detected Marking | Expiration date (or reject mark) determined by optical |
| | String | character recognition analysis of image of expiration |
| | | date marking on bag 26 |
| Process | Emboss Inspection | Start and stop time as well as pass/fail indication as to |
| Outcome | Outcome | whether inspection of marking indicates desired marking |
| | | was applied and is perceptible |

Outfeed Assembly 4100

| | | |
|---|---|---|
| Process | Final Disposition | Indication as to whether a given bag has been |
| Outcome | | determined to be acceptable or rejected |
| Process | Bag Placement | Start and stop time as well as pass/fail indication. |
| Outcome | Outcome | Indicated as failure if bag is detected to have released |
| | | out of gantry assembly 4200 before quality review |
| | | indicates release is acceptable |
| Bag Trait | Outfeed | Identifier for specific receptacle 4120A, B in outfeed |
| | Receptacle | assembly 4100 in which a bag 26 is stored |
| Date | Release Date | Date bag was removed from outfeed assembly 4100 by |
| | | user |
| Identifier | Released By | Identity of user who removed the bag 26 from the |
| | | system 10 |

Odometry data 6954 may be data related to the accumulated usage on certain components or assemblies within the system 10. The odometry data 6954 may also include temporal data relating to passage of time since specific events of interest (e.g. last system 10 cleaning). Example odometry data 6954 which may be tracked by example systems 10 described herein is provided in Table 4 below:

TABLE 4

| Attribute | Description |
|---|---|
| Elapsed Time Since Cleaning | Total time since last cleaning event |
| Time Since Pre-filter installed | Elapsed time since pre-filter 6204 was replaced |
| Elapsed Time Since ULPA | Elapsed time since last filter 6226 integrity test |
| integrity test | |
| Time Since ULPA Filter | Elapsed time since filter 6226 was replaced |
| Installed | |
| Time Since Exhaust Filter | Elapsed time since each exhaust filter 6238A, B, 6250 was |
| Installed | replaced |
| Accumulated UVC on time | Total time irradiator 3956 has been powered |
| (accumulator) | |
| Accumulated UVC on time | Total time irradiator 4010 has been powered |
| (polished) | |
| Total Flow through Carbon | Total amount of fluid transferred through currently installed |
| Filter | carbon filter (from input flow sensor 3910) |
| Accumulated UVC on time | Total time irradiation assembly 2608 has been powered |
| (fill station) | |
| Total Disinfects of Cartridge | Total number disinfection procedures performed or initiated |
| | on currently installed cartridge |
| Total Flow Through | Totalized flow through currently installed cartridge based on |
| Dispensed Fluid Flow Sensor | data from dispensing flow path flow sensor 4052A |
| 1 into Cartridge | |
| Total Flow Through | Totalized flow through currently installed cartridge based on |
| Dispensed Fluid Flow Sensor | data from dispensing flow path flow sensor 4052B |
| 2 | |
| Total Number of Bags | Total number of septa 393 pierced by dispensing sharp 2604 |
| Accessed by Cartridge | of a given cartridge 2606 |
| Time Since Pneumatic | Elapsed time since drier 6022 of the pneumatic distribution |
| Distribution Assembly Drier | assembly 6000 was last replaced |
| replaced | |
| Time Since Pneumatic | Elapsed time since replacement of each filter 6018, 6020, |
| Distribution Assembly filter | 6024 in the preparation assembly 6004 of the pneumatic |
| replaced | distribution assembly 6004. |

The supporting data 6956 may be data which supplements quality decision data 6952 and odometry data 6954. One type of supporting data 6956 may be data which assists a human user in monitoring or verifying function of the system 10. For example, annotated images (or video) from a particulate inspection system 3500 or imager 3733 of a marking assembly 3700 may form part of the supporting data 6956. Likewise, raw unprocessed image or video frames may be included. Supporting data 6956 may also include sensor data not included in the quality decision data 6952. This data may not be associated with predefined quality thresholds imposed on particular bags 26, but nonetheless tracked and stored. Supporting data 6956 may also include high granularity data such as the raw sensor outputs collected by the control system 15 in order to inform quality acceptance criteria determinations. Example supporting data 6956 which may be produced by example systems 10 described herein is provided below in Table 5:

TABLE 5

| Attribute | Description |
|---|---|
| Actual Yield | Actual percentage of theoretical yield & amount of yield of the |
| | product the batch includes |
| Annotated Inspection | Still(s) from video taken by particulate inspection system 3500 |
| Images | annotated with tracks and classifications for detected contents of |
| | interest |
| Inspection Log File | Log file containing ID's for detected contents of interest and |
| | respective classifications |
| Particulate Inspection | Video from particulate inspection system 3500 imagers 3518 |
| Videos | (optionally compressed) |
| Indicia Images | Images of indicia 56, 5014 taken by imagers when collecting |
| | component IDs |
| Outfeed Inventory | Image data from outfeed inventory imagers 4180 |
| Image Data | |
| Raw Sensor Data | Raw Data readouts from one or more sensors in system 10 |
| Raw imaging Data | Raw image or video frames from particulate inspection system 3500 |
| | imagers 3518, marking assembly 3700 images 3733, outfeed |
| | inventory imagers 4180 or other imagers of the system 10. |

A bag quality record 6960 may be created for each bag 26 processed by a system 10. A unique identifier may be assigned to (or read from) each bag 26 processed by a system 10 and a bag quality record file associated with the unique identifier may be built up as the bag 26 proceeds through the system 10. The bag quality record 6960 may be a data structure which contains a release decision for the bag 26 and all of the information used to support the release decision. The bag quality record 6960 may be stored in a non-volatile memory and updated by the control system 15 on a recurring basis (e.g. every 2 seconds or less) as a bag 26 is displaced through the system 10. Where required information has yet to be generated for a bag 26, the bag quality record 6960 may indicate that information as pending. Thus, in the event of a power failure, the bag quality record 6960 may be referenced to determine where in the production process a particular in process bag 26 was. Thus, the bag quality record 6960 may function as a bookmark allowing the system 10 to resume production of the bag 26 after power is restored. The data structure used for the bag quality record 6960 may be dictated by a master batch record for a particular type of bag 26 the system 10 is currently producing (e.g. 1 L bags of normal saline).

As shown, quality decision data 6952 and odometry data 6954 may be populated into the bag quality record 6960 data structure. The bag quality record 6960 may also include indications as to whether the bag 26 was produced in conformity with predefined acceptance criteria. The acceptance criteria may be defined in a master batch record specific to the bag 26 variety (e.g. 1 liter normal saline) being produced by a system 10. Acceptance criteria may, for instance, include acceptable ranges, thresholds, etc. for specific sensor data collected as a bag 26 is produced. Other acceptance criteria may specify that certain inspections match predefined expected results. Acceptance criteria may also include requirements that all process outcomes are deemed as a pass. Conformity may be determined by analysis of the bag quality record 6960 by the control system 15.

The bag quality record 6960 may be self-supporting. That is, a review of the bag quality record 6960 alone would provide sufficient justification to conclude the determined disposition for the bag 26 is appropriate. Some of the quality decision data 6952 may be generated by analysis of the contents of the bag quality record 6960 by the control system 15 at predefined points in production of a bag 26. This may inform the outcome of various checkpoint verifications mentioned above. For example, the control system 15 may analyze the existing information in the bag quality record 6960 and generate a pre-marking disposition. If all acceptance criteria have been conformed with prior to marking of the bag 26, an approval disposition may be noted as the checkpoint outcome. An expiration date may be applied to the bag 26 at a marking assembly 3700 as described elsewhere herein. Otherwise, a reject disposition may be noted in the bag quality record 6960 and a rejection marking may be applied to the bag 26. The control system 15 may similarly analyze the content of the bag quality record 6960 to generate a final disposition for the respective bag 26. An approval disposition may be noted if all acceptance criteria are conformed with and a reject disposition may be noted if at least one acceptance criteria is not met.

A batch record 6962 may be created for each bag 26 and may be associated with the same unique identifier for that bag 26 as the bag's bag quality record 6960. The bag quality record 6960 may also form part of the batch record 6962. The batch record 6962 may be a bag specific copy of the master batch record used for a particular variety of bag 26 (e.g. normal saline, half normal saline, dextrose, D5W, or any other medical fluid mentioned herein) produced by the system 10. The batch record 6962 may include the release decision from the bag quality record 6960. The supporting information stored in the bag quality record data structure may also be included. Certain odometry data 6954 not included in the bag quality record 6960 may be written to the batch record 6962. The batch record 6962 may also include the supporting data 6956. The batch record 6962 may further include information received from a database 6970 or information specified in a locally stored master batch record for the specific type of bag 26 being produced by the system 10. Example information which may be included in a batch record 6962 constructed by a system 10 is provided in Table 6 below:

TABLE 6

| Attribute | Description |
|---|---|
| Component List | Name of each component used to manufacture a bag 26 (e.g. product |
| | codes, product lot numbers, product expiration dates, description of |
| | components, and amount of each component used) |
| Component MFG ID | Identifier for suppliers of each components (and manufacturers if |
| | different) |
| Label Specimen | Representation of any label used on the bag 26 |
| Label Disposition | Pass/fail indication for label assigned by manufacturer |
| Label Grade | Quality indication for label determined e.g. as defined in ISO/IEC |
| | 15415 |
| Label Reviewer | Identity of reviewer (e.g. responsible personnel at manufacturer for |
| | bag 26) |
| Dispenser Date of | Date a specific dispenser 5000 used to produce batch was received by |
| Receipt | host site for system 10 |
| Dispenser Lot | Approval/Rejection determination from manufacturer for lot number |
| Disposition | to which a particular dispenser 5000 belongs |
| Bag Lot Disposition | Approval/Rejection determination from manufacturer for lot number |
| | to which a particular bag 26 belongs |
| Name, Strength, | The name of the solution in a given bag 26 along with relevant dosage |
| Dosage Form | information and dosage form information |
| Product Type | Description of drug product container and closure |
| Description | |
| Maintenance Log | Chronological record of cleaning/maintenance events performed on |
| | system 10 |
| Reviewer(s) | Identity of reviewer(s) for the master batch record for the drug product |
| | produced by the system 10 |
| Reviewer | Dated Signature(s) of Reviewer(s) |
| Signature(s) | |

Referring now to FIGS. 229A-C, a block diagram of an example fluid handling system 3900 which may be included in a system 10 is depicted. The exemplary fluid handling system 3900 receives water from a water source 3902 and may generate a diluent or medical excipient from that water. The fluid handling system 3900 routes the diluent to a fill station 2600 such as any of those shown or described herein. The diluent produced in the fluid handling system 3900 may be dispensed into a reservoir such as a bag 26 via a dispensing sharp 2604 of a cartridge 2606 (see, e.g., FIG. 101A). The bag 26 may contain a concentrate and the diluent may be dispensed into the bag 26 in a volume sufficient to generate a solution with a desired concentration from the concentrate in the bag 26. The fluid handling system 3900 may include a variety of water treatment devices which may purify the water from the water source 3902 to a meet a predetermined set of criteria. For example, the fluid handling system 3900 may purify water from the water source into USP (or some other pharmacopeia) water for injection (WFI). Other USP monograph waters may be produced in alternative embodiments. Other example waters may include water for hemodialysis.

The fluid handling system 3900 may include a plurality of sensors for monitoring fluid within the fluid handling system 3900. The control system 15 may use data from the sensors to ensure the diluent produced by the fluid handling system 3900 complies with a predetermined set of quality criteria. The plurality of sensors may provide data used to monitor for changes in functionality of certain components within the fluid handling system 3900. The sensors may also provide data used by the control system 15 to govern control of pumps, valves, heaters, and other components within the fluid handling system 3900.

Example fluid handling systems 3900 may produce disinfection fluid which may be used to disinfect portions of the fluid handling system 3900 as well as portions of a cartridge 2606 (see, e.g., FIG. 101A). Disinfection fluid may be used to disinfect connection interfaces with various replaceable or consumable components. For example, disinfection fluid may be used to disinfect the connection interface for a cartridge 2606. The disinfection fluid may also disinfect a portion of the consumable component being installed such as a section of a connection port 2812 (see, e.g., FIG. 126A of a cartridge 2606). The disinfection fluid may be heated diluent in certain examples.

Referring primarily to FIG. 229A, the fluid handling system 3900 may receive water from a water source 3902. The water source 3902 may be a source of potable water and in some examples may be a municipal drinking water supply. An inlet fluid pressure sensor 3904 and inlet fluid conductivity sensor 3906 may be included to generate data relating respectively to the pressure and conductivity of incoming fluid. The pressure sensor 3904 data may be monitored by the control system 15 to ensure that the water source 3902 is supplying water or supplying water above a predetermine inlet fluid pressure threshold or within a predefined acceptable fluid pressure range. In the event water from the water source 3902 is in breach of the threshold or range, an error may be generated by the control system 15.

Conductivity sensor 3906 and other conductivity sensors described herein may include a temperature sensor which may output temperature data. The control system 15 may analyze the temperature data and conductivity data from each conductivity sensor (e.g. 3906) in order to determine a conductivity of the fluid monitored by a given conductivity sensor. In the event incoming supply water is outside of a predefined temperature range or above a predefined conductivity threshold, the control system 15 may generate an error. Supply water input to the fluid handling system 3900 may be diverted to a drain (e.g. drain 3930A).

As shown, an inlet valve 3908 may be included with an input flow sensor 3910 being included downstream of the inlet valve 3908. Fluid may flow from the input flow sensor 3910 to at least one carbon filter 3912. The carbon filter 3912 may remove larger particulate as well as chlorine and chloramine from the incoming fluid. A sample valve 3914 may be disposed after the first carbon filter 3912 and may be commanded (e.g. when the control system 15 registers actuation of a dispense button) to an open state in order to dispense permeate from the carbon filter 3912 through a sample port 3918. The sample port 3918 may be accessible from an exterior of the enclosure 12 of a system 10. Fluid may be dispensed from the sample port 3918 into a reservoir 3920 to test for the presence of one or more contents of interest. For example, testing may be done on a periodic basis to ensure that the carbon filter 3912 is removing chlorine/chloramine from the incoming fluid.

A button which controls flow through the sample port 3918 may be disabled unless an input (e.g. via user interface 6100) is received requesting a sample be drawn from the fluid handling system 3900. The control system 15 may only toggle the sample valve 3914 to a flow permitting state after registering a button interaction in the event that various sampling criteria have been met. For example, the control system 15 may ensure that the data signals from the inlet pressure sensor 3904 and temperature data signal from the inlet fluid conductivity sensor 3906 are within predefined respective ranges. The conductivity data signal from the fluid inlet conductivity sensor 3906 may also be required to be below a predefined threshold. A total flow through the carbon filter 3912 (based on data from the input flow sensor 3910) may be checked to ensure that a least a predefined amount of fluid has passed through the carbon filter 3912 before sampling is permitted. Sampling may be performed manually with the results being entered into the system 10 via user interactions with the user interface 6100 (see, e.g., FIG. 1). In the event a test failure is indicated via the user interface 6100, the control system 15 may prohibit filling of bags 26 and place the system 10 in a standby state.

After passing through the one or more carbon filter 3912, the fluid may pass a post carbon filtration pressure sensor 3922. The control system 15 may monitor data from the post carbon filtration pressure sensor 3922 and compare this data to data from the inlet pressure sensor 3904 and inlet flow sensor 3910. The control system 15 may ensure that a pressure drop across the one or more carbon filter 3912 which is within an expected range is observed. In the event that the pressure drop is outside of the expected range, the control system 15 may prevent dispensing of fluid to a fill station 2606 and may require the carbon filter(s) 3912 be replaced. After passing the post carbon filtration pressure sensor 3922, fluid may flow to a pressure regulator 3924 which may regulate downstream fluid pressure to some predefined value.

In various implementations, a flush conduit 3926 may be included. When a flush valve 3928 on the flush conduit 3926 is in an open state, a flow path may be established to a flushing drain 3930A. Fluid may be passed through the carbon filter(s) 3912 to flush the carbon filter(s) 3912. The control system 15 may command the flush valve 3828 to the open state and flow at least a preset flushing volume of fluid from the water source 3902 through the carbon filter(s) 3912 each time one or more carbon filter 3912 is replaced. The control system 15 may orchestrate flushing of the carbon filter(s) 3912 by monitoring data from the input flow sensor 3910. For example, the control system 15 may flush the carbon filter(s) 3912 until data from the input flow sensor 3910 indicates at least a preset amount of fluid has passed through the carbon filter(s) 3912.

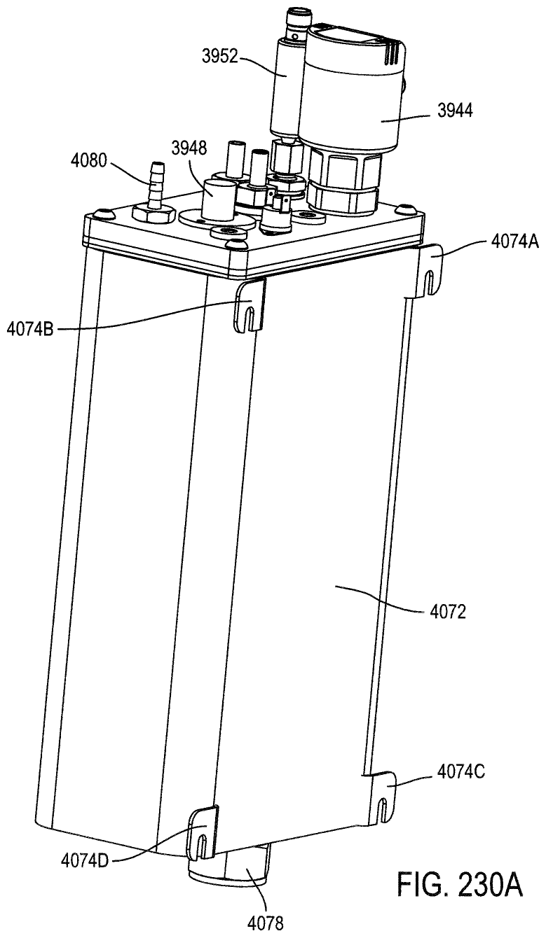

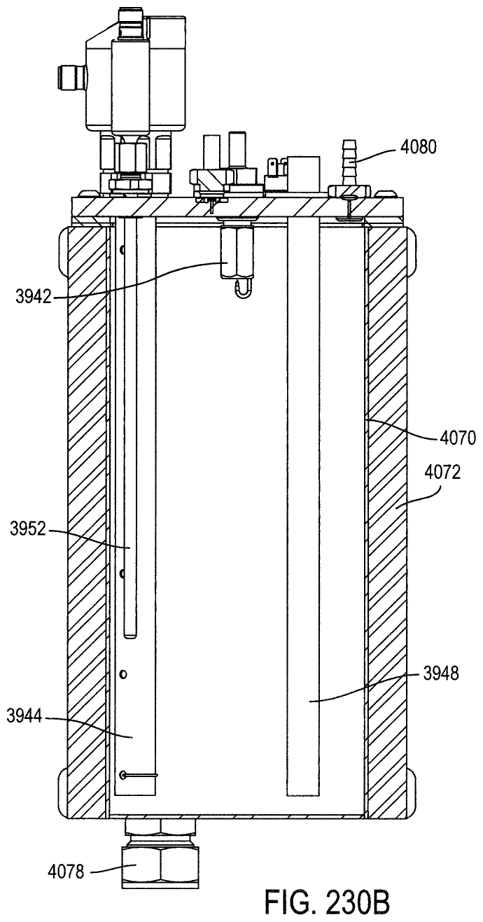

An accumulator inlet valve 3932 may be disposed downstream of the pressure regulator 3924. The control system 15 may close the accumulator inlet valve 3932 when flush valve 3928 is in an open state. The accumulator inlet valve 3932 may be opened to deliver fluid to an accumulator reservoir 3934 of the fluid handling system 3900. The example accumulator reservoir 3934 isolates the source pressure from the rest of the fluid handling system 3900. The accumulator reservoir 3934 may also be heated. This may kill microbes, inhibit microbial growth, and assist in deaerating water introduced to the fluid handling system 3900. The example accumulator reservoir 3934 assists in generating disinfection fluid by providing a source of elevated temperature water. An example accumulator reservoir 3934 is described in further detail in relation to FIGS. 230A-B.

As shown, the accumulator reservoir 3934 may be paired with one or more heat exchanger 3936. Fluid delivered to the accumulator reservoir 3934 may be routed through the heat exchanger(s) 3936 to recover heat from the fluid exiting the accumulator reservoir 3934. This will increase the temperature of fluid being introduced to the accumulator reservoir 3934 allowing the heating of fluid at the accumulator reservoir 3934 to be more efficient. The heat exchanger(s) 3936 also decrease the temperature of fluid leaving the accumulator reservoir 3934. Typically, fluid dispensed from the fill station 2600 may be in a range of 25-40° C. The heat exchanger(s) 3936 may reduce the temperature of fluid exiting the accumulator reservoir 3934 to at least the upper bound of this range. The heat exchanger(s) 3936 may be any suitable variety of heat exchanger 3936 though plate type heat exchangers may be particularly advantageous due to their compactness.

The fluid handling system 3900 may include a bypass flow path 3940. The bypass flow path 3940 may allow fluid to be routed around the heat exchanger(s) 3936 depending on the state of a heat exchanger bypass valve 3938. This may allow fluid exiting the accumulator reservoir 3936 to be kept at elevated temperature and used as a disinfect fluid.

As fluid enters the accumulator reservoir 3934, it may pass through a mister or atomizer 3942. As shown, an accumulator heater 3948 may additionally be included in the accumulator reservoir 3934. The atomizer 3942 in conjunction with the heat from the accumulator heater 3948 help to remove dissolved gases from the incoming water. The example accumulator reservoir 3934 includes a vent flow path 3946 which may also serve as an overfill relief flow path. The vent flow path 3946 may be in communication with an accumulator drain 3930B or other discard reservoir. Gas from deaeration of the incoming water may be vented through the vent flow path 3946.

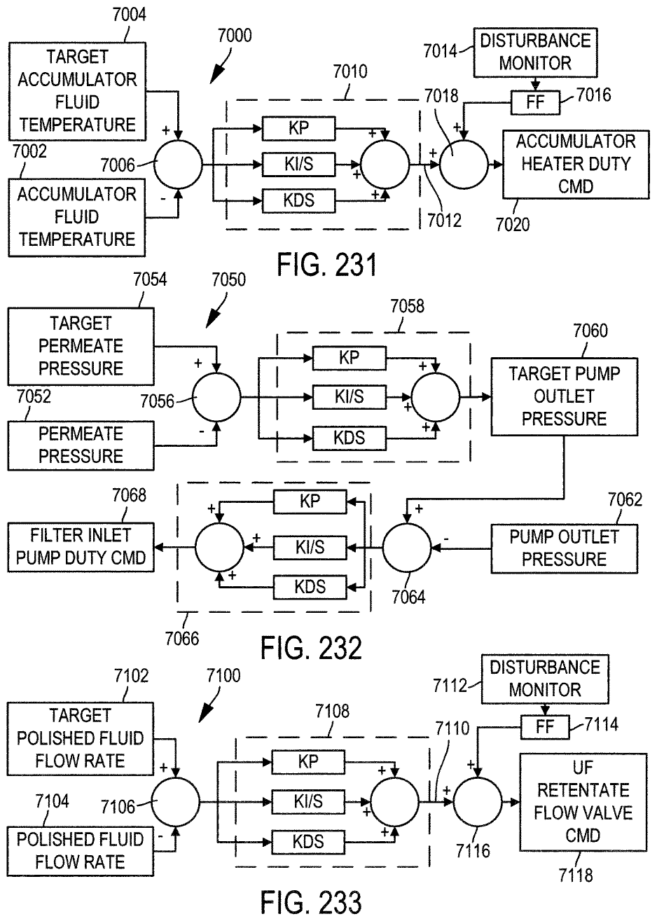

The accumulator heater 3948 may be powered to heat fluid within the accumulator 3934 to a predefined temperature value (or within a preset range of a predefined value. Signals from one or more accumulator temperature sensor 3952 may be utilized by the control system 15 to set and adjust a duty cycle for the accumulator heater 3948. For example, the control system 15 may alter a PWM command to the heater using a P, PI, or PID control loop informed by the accumulator temperature sensor 3952 data signal. In some examples, the predefined temperature value targeted for fluid in the accumulator 3934 may be 75-85° C. (e.g. 80° C.). An example temperature control diagram for the accumulator heater 3948 is depicted in described in relation to FIG. 231.

A heater temperature sensor 3950 may also be included and the control system 15 may shut off power to the accumulator heater 3948 in the event that the heater temperature sensor 3950 indicates the accumulator heater 3948 is in breach of a predetermined over temperature threshold. Fluid exits the accumulator reservoir 3934 via an accumulator outlet flow path 3953 and passes to the heat exchanger(s) 3936.

A level sensor 3944 may be included and may output a signal which varies in relation to the level of a liquid held within the accumulator reservoir 3934. The level sensor 3944 may be any suitable variety such as a float type level sensor. The level of liquid within the accumulator reservoir 3934 may, for example, be maintained within a fill level range. The control system 15 may use data from the level sensor 3944 to govern operation of the accumulator fill valve 3932. This may facilitate maintaining the level of fluid within the accumulator reservoir 3934 at or within a range of a preset target level. In various examples, a bang-bang type controller may be implemented. The accumulator fill valve 3932 may be toggled to a flow permitting state when the level output signal from the level sensor 3944 drops below a preset low level threshold. The accumulator fill valve 3932 may be toggled closed when the output signal from the level sensor 3944 breaches a preset high level threshold. An error may be generated by the control system 15 in the event that the output signal from the level sensors 3944 indicates that the level in the accumulator 3934 is not changing as expected. The control system 15 may generate a notification (e.g. via the user interface 6100) and place the system 10 in a standby state. For example, if the level has remained below the low level threshold for more than a predefined period of time (e.g. 15 seconds) with the accumulator fill valve 3932 in the open state.

Referring now also to FIG. 229B, fluid may be drawn from the accumulator reservoir 3934 by powering a filter inlet pump 3954. Preferably, the accumulator reservoir 3934 may be placed above the filter inlet pump 3954 such that the head height of the accumulator reservoir 3934 provides fluid at positive pressure to the filter inlet pump 3954. This may help prevent cavitation within the fluid as the filter inlet pump 3954 is powered.

As fluid is drawn from the accumulator 3934, it may pass at least one irradiator 3956. The irradiator 3956 may include one or more UV lights, for example, LEDs which emit light in a UVC wavelength. Upstream of the irradiator 3956 may be at least one heat exchanger output temperature sensor 3957. The control system 15 may ensure that fluid leaving the heat exchanger 3936 is within an acceptable range based on data from the heat exchanger output temperature sensor 3957. The acceptable range may be determined based on the irradiator 3956 selected. This may ensure that the lumen output of the irradiator 3956 is above a preset threshold.

The filter inlet pump 3954 may be powered to drive fluid through a filter assembly 3962. The filter inlet pump 3954 may operate at a preselected duty cycle or under one or more different control loop. The control mode of the filter inlet pump 3954 may be switched depending on the status of the fluid handling system 3900. For example, the filter inlet pump 3954 may be run at a preset duty cycle during initial priming of the fluid handling system 3900. Control of the filter inlet pump 3954 may be transitioned to a control loop, after, for example, the recirculation flow path 3972 is determined to have been primed. The control loop may for example determine the error between a pump outlet pressure target and the measured outlet pressure from a pump outlet pressure sensor 3958. This may be fed to an inlet pump PID controller which may generate a duty cycle command for the filter inlet pump 3954. As priming of the fluid handling system 3900 proceeds, the filter inlet pump 3954 may be transitioned to a cascade control loop which accounts for pressure on the permeate side of the filter assembly 3962 in addition to the pump outlet pressure. Such a control loop is described in greater detail in relation to the example control diagram 7050 depicted in FIG. 232.

In the example embodiment, the filter assembly 3962 includes a reverse osmosis filter 3964 and a filtration assembly heater 3966. The filter assembly heater 3966 may be a blanket type heater that at least partially surrounds the filter 3964 and fluid paths therethrough. In certain examples, the filtration assembly heater 3966 may be unpowered except when the fluid handling system 3900 is producing disinfect fluid. A filtration assembly heater temperature sensor 3968 and a filter temperature sensor 3970 may be included in the filter assembly 3962. The control system 15 may alter a PWM command to the heater using a P, PI, or PID control loop informed by filter temperature sensor 3970 data signal and a fluid temperature set point to maintain the filter assembly 3962 at or within a range of the desired temperature set point. The control system 15 may shut off power to the filtration assembly heater 3966 in the event data from the filtration assembly heater temperature sensor 3968 indicates the filtration assembly heater 3966 temperature is in breach of an over temperature threshold. Intermediate the filter inlet pump 3954 and filter assembly 3962 may be a pump outlet pressure sensor 3958 as mentioned above and a conductivity sensor 3960.

Retentate from the filter assembly 3962 may pass to a recirculation flow path 3972 and may return to the filter inlet pump 3954 when a retentate flow valve 3978 is in an open state. Retentate may alternatively be delivered to a retentate drain 3930C when a retentate drain valve 3974 is transitioned from a closed to an open state. A drain flow rate sensor 3976 may output a signal which varies in relation to the flow rate of retentate to the drain 3930C when the retentate drain valve 3974 is in an open state.

Retentate passing from the filter assembly 3962 to the recirculation flow path 3972 may be monitored for pressure, flow rate, temperature, and conductivity. At least one retentate pressure sensor 3980, retentate flow rate sensor 3982, and conductivity sensor 3984 (which may also monitor and output temperature data) may be included for this purpose. A check valve 3986 may be included to ensure one directional flow within the recirculation flow path 3972. The control system 15 may monitor data from the retentate conductivity sensor 3984 and may adjust the state of the retentate drain valve 3974 to direct the retentate stream to the retentate drain 3930C when the conductivity of the retentate rises above a predefined threshold.

The flow rate of the retentate stream may be kept relatively high. The high retentate flow may rinse the input side of the filter 3964 membrane mitigating concentration polarization and related concerns. The control system 15 may monitor data from the retentate flow rate sensor 3982 and adjust the state of the retentate flow valve 3978 in order to achieve a target retentate flow rate or maintain the flow rate within a predetermined range thereof. The control system 15 may alter a position command to the retentate flow valve 3978 using a P, PI, or PID control loop informed by a target retentate flow rate and the flow rate sensor 3982 data signal to maintain a desired or target retentate flow. Similarly, a position command may be determined by the control system 15 for the retentate drain valve 3974. A P, PI, or PID control loop may output a position command for the retentate drain valve 3974 using the error between a target retentate drain flow rate and that indicated by the output signal of the retentate drain flow sensor 3976. The retentate flow valve 3978 and the retentate drain valve 3974 may also be controlled to a preset position in certain scenarios (e.g. during priming of the fluid handling system 3900).

Permeate from the filter assembly 3962 may pass from the filter assembly 3962 to a permeate flow path 3989. As shown, the pressure, flow rate, temperature, and conductivity of fluid may be sensed on the permeate flow path 3989. A permeate pressure sensor 3990, permeate flow rate sensor 3992, and permeate conductivity sensor 3994 (which may also monitor and output temperature data) may be included for this purpose.

Certain embodiments may include a pressure relief valve 3996 on the permeate flow path 3989. The pressure relief valve 3996 may be opened to establish a path to a pressure relief drain 3930D. The pressure relief valve 3996 may be selected to automatically open at a predetermined pressure (e.g. 45 p.s.i.). The opening pressure for the pressure relief valve 3996 may be chosen to be lower than a maximum operating pressure of a downstream component. In certain examples, opening pressure may be lower than the lowest maximum operating pressure of any downstream component.

The permeate flow path 3989 may extend to one or more polishing unit 3988. An electrodeionization unit may be included as the polishing unit 3988 in certain examples. In such embodiments, after passing a check valve 3998, the permeate stream may furcate into a number of input flow streams to the electrodeionization unit. As shown, the permeate flow path 3989 may furcate into a feed stream flow path 4000, an electrode stream flow path 4002, and a concentrate stream flow path 4004. The product output of the polishing unit 3988 may be coupled to a polished fluid flow path 4014.

An electrode stream flow sensor 3999 may be included and may monitor the flow rate of fluid through the electrode stream flow path 4002. A polished fluid flow sensor 4019 may also monitor product flow exiting the electrodeionization unit through the polished fluid flow path 4014. The total flow to the electrodeionization unit indicated by the permeate flow rate sensor 3992, the electrode flow sensor 3999 output, and polished fluid flow sensor 4019 may be used to determine the concentrate stream flow rate.

Flow controllers 4006A-B may be included on each of the electrode stream flow path 4002 and concentrate stream flow path 4004. The flow controllers 4006A-B may set the flow of fluid into the electrode and concentrate flow paths 4002, 4004. In certain examples, the flow controllers 4006A, B may be orifice type flow restrictors. Valves which may be toggled between open and closed states by the control system 15 may alternatively be included.

Fluid from the electrode stream flow path 4002 may exit the polishing unit 3988 and be directed to a drain 3930E. A check valve 4008 may be included to prevent backflow. Fluid from the concentrate stream flow path 4004 may exit the polishing unit 3988 and be delivered to a drain 3930F. A check valve 4009 may be included to prevent backflow. Alternatively, the electrode and concentrate outputs may be plumbed to the same drain 3930E. In alternative embodiments, the concentrate stream flow path 4004 may be plumbed to return to the inlet side of the filter inlet pump 3954, accumulator reservoir 3934, or a dedicated break tank from which the filter inlet pump 3954 may draw.

Fluid from the feed stream flow path 4000 may exit the polishing unit 3988 and pass to polished fluid flow path 4014. At least one irradiator 4010 may be disposed intermediate the polishing unit 3988 and depyrogenation unit 4012. Example irradiators 4010 may, for example, emit UVC light and include one or more LED. In certain embodiments, the irradiator 4010 may be the same as irradiator 3956. The irradiator 4010 may be paired with a temperature sensor upstream of the irradiator 4010 in certain examples. The control system 15 may ensure that product fluid leaving the polishing unit 3988 is within an acceptable temperature range based on data from this temperature sensor. This may ensure that the lumen output of the irradiator 4010 is above a preset threshold.

A polished fluid flow sensor 4019, polished fluid pressure sensor 4020, and polished fluid pre-heater temperature sensor 4022 may monitor fluid in the polished fluid flow path 4014 upstream of a polished fluid heater 4016.

A polished fluid output temperature sensor 4024 may monitor the temperature of fluid in the polished fluid flow path 4014 downstream of the polished fluid heater 4016. A polished fluid heater temperature sensor 4026 may also be included. The control system 15 may power the polished fluid heater 4016 to heat the polished fluid to or within a range of a predetermined temperature set point. In certain examples, the polished fluid heater 4016 may be unpowered except when the fluid handling system 3900 is producing disinfect fluid. The control system 15 may generate a duty cycle command to the polished fluid heater 4016 based on data signals from the polished fluid flow sensor 4019, polished fluid pre-heater temperature sensor 4022, and polished fluid output temperatures sensor 4024. The control system 15 may also monitor the data signal from the polished fluid heater temperature sensor 4026 and shut off power to the polished fluid heater 4016 in the event that a predefined over temperature threshold is breached.

Fluid in the polished fluid flow path 4014 may flow to a depyrogenation unit 4012 at the downstream end of the polished fluid flow path 4014. The depyrogenation unit 4012 in the example embodiment includes at least one ultrafilter. In examples where the depyrogenation unit 4012 includes one or more ultrafilter, retentate from the ultrafilter(s) may pass to a return flow path 4028. A check valve 4030 may be included to ensure flow in the return flow path 4028 is unidirectional. A return flow valve 4032 may be included on the return flow path 4028 to control flow of fluid through the return flow path 4028. Fluid in the return flow path 4028 may be fed back to the filter assembly 3962 via filter inlet pump 3954.

The return flow valve 4032 may be controlled by the system 10 to keep the flow through the filter assembly 3966 and polishing unit 3988 substantially constant when fluid is being dispensed into a bag 26 from the fluid handling system 3900. During dispensing of fluid through a cartridge 2606, the retentate fluid from the ultrafilter may be decreased. This may help to keep pressure stable in the portion of the fluid handling system 3900 upstream of the ultrafilter(s). The return flow valve 4032 may be controlled using a P, PI, or PID control loop informed by a target polished fluid flow rate and the polished fluid flow rate indicated by the polished fluid flow sensor 4019. The flow rate indicated by the dispensed fluid flow rate sensors 4052A, B (e.g. the average of the flow rate measured by each) may be used to inform a feed forward term used to adjust the return flow valve 4032 command output by the return flow valve control loop. An example control diagram 7100 for the return flow valve 4032 is depicted in FIG. 233. The return flow valve 4032 may also be controlled to a set point during certain scenarios (e.g. during priming of the fluid handling system 3900).

Referring now also to FIG. 229C, permeate from the ultrafilter may pass to a dispensing flow path 4044 in a dispensing portion of the fluid handling system 3900. Fluid in the dispensing portion of the fluid handling system 3900 may be subjected to various quality characteristic sensing and directed either to a drain 3930G, H or dispensing sharp 2604 (see, e.g., FIG. 139) as appropriate. Fluid passing from the depyrogenation unit 4012 may be monitored for pressure and temperature by respective pressure and temperature sensors 4034, 4036. In examples including an ultrafilter, the control system 15 may check the dispensing portion inlet pressure sensor 4034 against the polished fluid pressure sensor 4020. In the event that a pressure drop less than a predetermined amount is detected, the control system 15 may prohibit delivery of fluid to the fill station 2600 and fluid may be directed to a drain 3930G. The control system 15 may generate a notification to change the depyrogenation unit 4012. The control system 15 may require confirmation that the depyrogenation unit 4012 has been replaced and may orchestrate a disinfect cycle for the fluid handling unit 3900 prior to allowing fluid to be routed to the fill station 2600. Provisioning of fluid to the fill station 2600 may be prohibited until the depyrogenation unit 4012 is replaced and disinfected.

A total organic carbon (TOC) sensor 4038 may be included in the fluid handling system 3900. In the example, the TOC sensor 4038 includes a conductivity sensor 4040 and temperature sensor 4042 to assist in generating the TOC data. The TOC sensor 4038 is provided on a slipstream 4046 off the dispensing flow path 4044 in the example depicted. The slipstream 4046 may be or may include a heat exchanger 4047. The heat exchanger 4047 may lower the temperature of the fluid en route to the TOC sensor 4038 to a temperature below the maximum operating temperature for the TOC sensor 4038. As mentioned above, high temperature disinfect fluid may be generated by the fluid handling system 3900. The heat exchanger 4047 may lower the temperature of this fluid below 70° C. (e.g. below 60-65° C.). In some embodiments, the slipstream 4046 may be or include a coil of metal (e.g. stainless steel) tubing which acts as the heat exchanger 4047. TOC data from the TOC sensor 4038 may be compared to a predefined threshold by the control system 15. In the event that the TOC data is in breach of the threshold, fluid may be directed to a drain 3930G and inhibited from passing to the fill station 2600.

A flow control valve 4048 may be included downstream of the furcation of the slipstream 4046 off of the dispensing flow path 4044. One or more sets of redundant sensors may be included downstream of the flow control valve 4048. In the example embodiment, a set of conductivity sensors 4050A, B are included. The conductivity sensors 4050A, B may be ultrapure water conductivity sensors in certain examples. The control system 15 may compare data from the conductivity sensors 4050A, B to ensure that the difference in conductivity indicated by data from each of the sensors 4050A, B is below a predetermined threshold. In the event the data from the conductivity sensors 4050A, B diverge from one another a sufficient amount to breach the threshold, the control system 15 may direct fluid in the dispensing flow path 4044 to a drain 3930G. Fluid may similarly be routed to a drain 3930G if the data from the conductivity sensors 4050A, B indicates that the conductivity of fluid in the dispensing flow path 4044 is in excess of a predefined value. The bag 26 in place at the fill station 2600 may be flagged by the control system 15. After the bag 26 is removed from the fill station 2600, the bag 26 may be marked as rejected. This may, for example, be accomplished with a marking assembly 3700 such as that depicted in FIG. 201. The bag 26 may be placed in a quarantine location after marking (see, e.g., receptacles 4120B of FIG. 216).

The control system 15 may prohibit filling of another bag 26 at the fill station 2600 until a flush of the dispensing flow path 4044 and cartridge 2606 has completed. The control system 15 may displace a prime line 3120 to the cartridge 2606. Example prime lines 3120 are further described in relation to FIG. 128. Fluid may be delivered into the prime line 3120 at least until the conductivity sensors 4050A, B indicate the conductivity of the fluid is below a threshold. A predefined flushing volume may be transferred through the cartridge 2606 and into the prime line 3120 after the conductivity value has decreased below the predefined conductivity threshold. A set of flow rate sensors 4052A, B may be monitored to determine when the flushing volume has been delivered. The prime line 3120 may then be removed and the control system 15 may resume filling of bags 26 at the fill station 2600.

A set of flow rate sensors 4052A, B may also be utilized to ensure bags 26 are filled to a desired amount. The control system 15 may monitor the signal from the flow rate sensors 4052A, B. Fluid may continue to be transferred into a bag 26 through the cartridge 2606 until the flow rate sensors 4052A, B indicate a target amount of fluid is dispensed into a bag 26 in place at the fill station 2600. As with the conductivity sensors 4050A, B, the control system 15 may also compare the data from each flow rate sensor 4052A, B. The control system 15 may prohibit filling of bags 26 in the event that the data from the flow rate sensors 4052A, B diverge by more than a certain amount. A set of cartridge input temperature sensors 4054A, B and a cartridge input pressure sensor 4056 may also be included downstream of the flow control valve 4048.

In some embodiments, the target amount of fluid may be a mass. The control system 15 may convert the flow rate indicated by flow rate sensors 4052A, B to a mass flow rate using a density determination informed by other sensors in the fluid handling system 3900. For example, the data from the cartridge input temperature sensor 4054A, B and potentially the cartridge input pressure sensor 4056 may be used to determine a density. In applications where impact of the pressure would make a negligible impact, the pressure contribution may be ignored.

Additionally or alternatively, one or more mass sensor may be included to monitor the mass of the bag 26 as the bag is filled. For example, the port retainers 3146 on the fill displacement stage 3142 (see, e.g., FIG. 130) may be coupled to the remainder of the fill displacement stage 3142 via a coupling including at least one strain gauge. One more load cell could alternatively be incorporated into the fill displacement stage 3142 to monitor the mass of the bag 26 in place on the port retainers 3146. Any mass data relating to the bag 26 may be monitored by the control system 15 to determine when the bag 26 has been filled to target amounts.

Still referring to FIGS. 229A-C, fluid may be delivered from the dispensing flow path 4044 to an interface for a cartridge 2606 (see, e.g., FIG. 126B). In the example embodiment, the interface is shown as a receptacle 2818 of a supply manifold 2820 (further described elsewhere herein, see, e.g., FIGS. 126A-B). At least some of the fluid characteristic sensors 4050A, B, 4052A, B, 4054A, B, 4056 may be included in the supply manifold 2820 in certain embodiments. A supply manifold 2820 including a cartridge input temperature sensor 4054B and a cartridge input pressure sensor 4056 is depicted in FIG. 107.

As further described in relation to FIGS. 126A-B, a cartridge 2606 may include a connection port 2812 which may be covered by a frangible 2816. The connection port 2812 may be advanced into the receptacle 2818 an amount sufficient for a sealing member of the connection port 2812 to seal against the walls of the receptacle 2818 downstream of a disinfection outlet flow path 2824. Disinfection fluid may be delivered to the connection interface and may exit the connection interface through the disinfection outlet flow path 2824. This may bathe the frangible 2816 and a portion of the connection port 2812 in disinfection fluid. The cartridge input temperature sensors 4054A, B may be monitored to ensure that the disinfection fluid temperature is within a target set point or range at a point proximate the connection interface. The target temperature may be 75-85° C. (or 80° C. where a set point is used). Once the frangible 2816 and portion of the connection port 2812 have been bathed in disinfection fluid for a predetermined period of time, the dispensing sharp 2604 may be placed into fluid communication with the dispensing flow path 4044 via the cartridge 2606. This may be accomplished by further advancing the connection port 2812 within the receptacle 2818 until a spike 2822 in the receptacle 2818 punctures the frangible 2816. At least one sealing member on the connection port 2812 may be displaced upstream of the disinfection outlet flow path 2824 as the connection port 2812 is spiked. This may block flow from the dispensing flow path 4044 to the disinfection outlet flow path 2824.

During disinfection of the connection port 2812 and frangible 2816, fluid passing through the disinfection outlet flow path 2824 may be routed to a drain 3930H. Fluid delivered to the prime line 3120 may also be communicated to the drain 3930H. In some examples, there may be a valve upstream of the connection interface which may route fluid to the drain flow path 4060. This valve may be toggled by the control system 15 to direct fluid into the drain flow path 4060 in the event that data from one of the fluid characteristic sensors 4050A, B, 4052A, B, 4054A, B, 4056 indicates the fluid in the dispensing flow path 4044 is out of correspondence with a respective predefined criteria. As shown, a flow selector valve 4058 may be included and may control flow into the drain flow path 4060. The flow selector valve 4058 may close flow to the drain flow path 4060 or permit flow from either the prime line 3120 or dispensing fluid flow path 4044. A drain pressure sensor 4062 may be included on the drain flow path 4060 and may output a signal indicative of fluid pressure with the drain flow path 4060. A drain pump 4064 may also be in the drain flow path 4060. The drain pump 4064 may be powered to draw fluid out of a cartridge 2606 or prime line 3120. Fluid may be drawn out of the cartridge 2606 before the cartridge 2606 is removed from the fill station 2600. The drain pump 4064 may additionally be run during disinfection of the connection port 2812 for a newly installed cartridge 2606.

Referring now to FIGS. 229A-C, the fluid handling system 3900 may also generate and route disinfect fluid through the fluid handling system 3900 to disinfect the fluid handling system 3900. This may be done on some periodic basis and/or each time the cartridge 2606 or a filter of the fluid handling system 3900 is replaced. The disinfect fluid may be high temperature water in certain examples.

Referring now to FIGS. 230A-B, a perspective view of an example accumulator reservoir 3934 and a cross-sectional view showing the interior thereof are respectively shown. As show, the example accumulator reservoir 3934 includes a housing 4070 defining the interior volume of the accumulator reservoir 3934. The interior volume may vary from embodiment to embodiment, though in some embodiments, may be at least double the capacity of a reservoir (e.g. bag 26) to be filled at the filling station 2600 of the system 10. The interior volume may be two liters in some examples. The housing 4070 may be surrounded at least partially by insulation 4072. The housing 4070 includes mounting brackets 4074A-D for coupling the accumulator reservoir 3934 to a portion of an enclosure 12 of the system 10.

The accumulator reservoir 3934 includes an atomizer 3942 through which fluid may be fed into the accumulator reservoir 3934. The housing 4070 includes an interface panel 4076 which may be coupled to the rest of the housing 4070 with a gasket 4078 therebetween. A level sensor 3944, accumulator temperature sensor 3952, and accumulator heater 3948 may extend into the interior volume of the accumulator reservoir 3934 via the interface panel 4076. A vent fitting 4080 may also be included on the interface panel 4076 for coupling to the vent flow path 3946. An outlet port 4078 is also depicted and may be positioned at a low point or at the bottom of the accumulator reservoir 3934.

Referring now to FIG. 231, an example control diagram 7000 for an accumulator heater 3948 (see, e.g. FIG. 229A) is depicted. As shown, the accumulator fluid temperature indicated by the output signal of the accumulator temperature sensor 3952 may be passed to a summer 7006. The target accumulator fluid temperature 7004 may be also be input to the summer 7006. The target accumulator fluid temperature 7004 may be a predefined value. In some examples, the target accumulator fluid temperature 7004 may be at least 80° C. In some embodiments, the target accumulator fluid temperature 7004 may be dependent on whether the fluid handling system 3900 is producing disinfect fluid or fluid to fill bags 26. The target accumulator fluid temperature 7004 when producing disinfect fluid may be 90° C. or more.

Summers described herein, including summer 7006, combine their various inputs into an output. Use of the word “summer” anywhere herein shall not be construed to mean addition only must be performed, only that various inputs are combined into an output. At the summer 7006, a difference between the accumulator fluid temperature 7002 and the target 7004 may be determined. This output or error value may be passed to a PID controller 7010 which outputs a raw accumulator heater duty cycle command 7012. It should be noted that the gains used for the proportional, integral, and derivative terms of the PID controller 7010 may vary depending on the embodiment, and at least one may potentially be set to zero (e.g. the derivative term). In some embodiments, at least one disturbance monitor 7014 may be employed in control of the accumulator heater 3948. A disturbance monitor 7014 which monitors the state of the accumulator inlet valve 3932 may, for instance, be utilized. A feed forward controller 7016 may generate a disturbance compensation output based on data from the disturbance monitor 7014. This disturbance compensation output may be combined with the raw accumulator heater duty cycle command 7012 at summer 7018. The contribution from the feed forward controller 7016 may be zero in the event that the accumulator inlet valve 3932 is closed and may be a preset adjustment when the accumulator inlet valve 3932 is open.

In some examples, a disturbance monitor 7014 which monitors the temperature of the incoming source water 3902 may be included. A feed forward contribution from a feed forward controller informed by an inlet temperature disturbance monitor may increase as the temperature of the source water 3902 decreases. The temperature for the incoming source water 3902 may be read from the temperature output signal of the input fluid conductivity sensor 3906. Additionally or alternatively, a disturbance monitor 7014 which monitors the flow rate of incoming fluid may be included. A feed forward contribution from a feed forward controller informed by an inlet fluid flow rate may increase as the flow rate of source water 3902 increases. The flow rate of the incoming source fluid may be read from the input fluid flow sensor 2910. Any feed forward contributions may be combined with the raw accumulator heater duty cycle command 7012 in the summer 7018. The summer 7018 may output a final heater duty cycle command 7020 which may be used to control the accumulator heater 3948.

Referring now to FIG. 232, an example control diagram 7050 for a filter inlet pump 3954 is depicted. The example control diagram 7050 governs operation of the filter inlet pump 3954 with a cascade control loop. As mentioned elsewhere herein, the filter inlet pump 3954 may also be operated at a predefined set point or controlled with a control loop informed by a target and sensed input pressure to the filter assembly 3966. The control regime for the filter inlet pump 3954 may switch depending on the state of the fluid handling system 3900. In general, after the fluid handling system 3900 has been primed, a cascade style control loop such as that described in relation to FIG. 232 may be preferred.

As shown in FIG. 232, the permeate pressure 7052 indicated by the output signal of the permeate pressure sensor 3990 and a target permeate pressure 7054 may be fed to a summer 7056. The target permeate pressure 7054 may be predefined for various modes of operation for the fluid handling system 3900 (e.g. normal water production, disinfect, priming, etc.). A difference between the permeate pressure 7052 and the target permeate pressure 7054 may be determined via the summer 7056. This output or error value may be passed to a PID controller 7058 which outputs a target filter inlet pump output pressure 7060. Set point ramping may also be utilized for the output of the PID controller 7058. In certain examples, adjustments to the target filter inlet pump output pressure 7060 may be rate limited. A difference between the target filter inlet pump output pressure 7060 and the filter assembly input pressure 7062 may be computed at summer 7064. The filter assembly input pressure may be read from the output signal of the pump outlet pressure sensor 3958. The difference from the summer 7064 may be input to a PID controller 7066 for the filter inlet pump 3954. A filter inlet pump duty cycle command 7068 may be output by the PID controller 7066 and may be used to govern operation of the filter inlet pump 3954. Ramping may be employed to prevent abrupt changes. Adjustments to the filter inlet pump duty cycle command 7068 may be rate limited for example. It should be noted that the gains used for the proportional, integral, and derivative terms of the PID controllers 7058, 7066 may vary depending on the embodiment, and at least one may potentially be set to zero (e.g. the derivative term).

Where control of various components in the fluid handling system 3900 switches between multiple control loops or from being controlled at a defined set point to a closed loop type control, the transition may be bumpless. Set point tracking and control variable tracking may be used. This may eliminate sudden changes when the control regime is switched to a closed loop arrangement or between different varieties of closed loop arrangements.

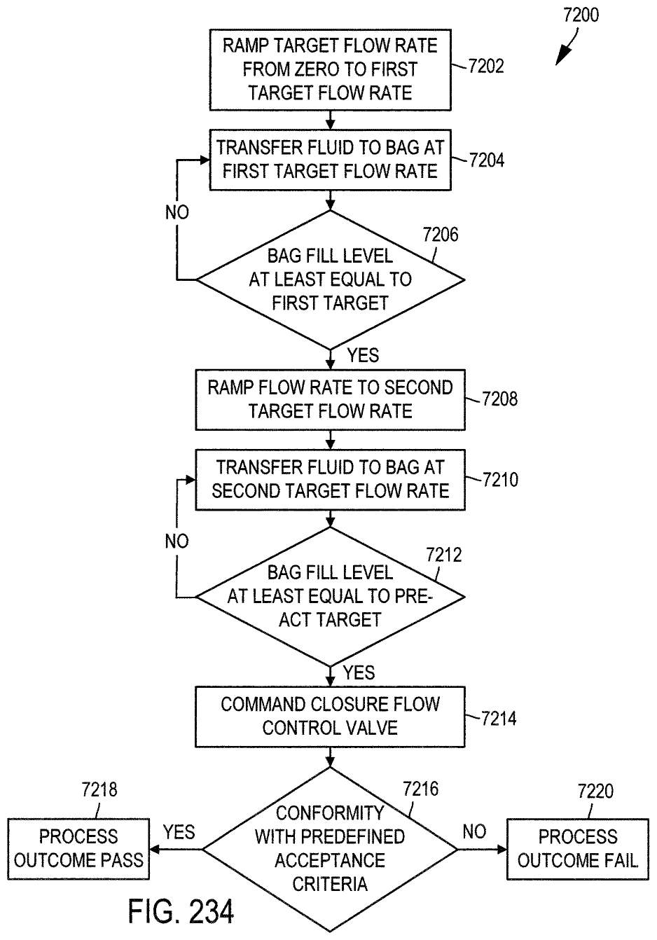

Referring now to FIG. 233, an example control diagram 7100 for a return flow valve 4032 is depicted. As shown, a target polished fluid flow rate 7102 and the polished fluid flow rate 7104 indicated by the output signal of the polished fluid flow sensor 4019 may be passed to a summer 7106. The target polished fluid flow rate 7102 may be a preset. The value of the target polished fluid flow rate 7102 may be dependent on whether the fluid handling system 3900 is producing disinfect fluid or fluid for filling of reservoirs. A difference between the target polished fluid flow rate 7102 and the polished fluid flow rate 7104 may be output by the summer 7106 to a PID controller 7108 for the return flow valve 4032. It should be noted that the gains used for the proportional, integral, and derivative terms of the PID controller 7108 may vary depending on the embodiment, and at least one may potentially be set to zero (e.g. the derivative term). The PID controller 7106 may output a raw return flow valve command 7110. The raw return flow valve command 7110 may be subject to a feed forward adjustment. As shown, a disturbance monitor 7112 is included and may feed data to a feed forward controller 7114. The disturbance monitor 7112 may monitor flow through the dispensing flow path 4028 based on the output signals from flow sensors 4050A, B (e.g. the average of the flow sensed by these sensors). The feed forward controller 7114 may generate a disturbance compensation output which serves to decrease the flow through the return flow path 4028 when fluid is being drawn out of the fluid handling system 3900 to fill a bag 26. The compensation output may adjust based on the mass flow rate into each bag 26 as the bag 26 is being filled (further described in relation to FIG. 234). This may help to keep the flow through the permeate flow path 3989 and the polished fluid flow path 4014 substantially stable when fluid is dispensed through a cartridge 2606. The output of the feed forward controller 7114 may be applied to the raw return flow valve command 7110 at summer 7116. Summer 7116 may output a final return flow valve command 7118 which may be used by the control system 15 to govern operation of the return flow valve 4032.

Referring now primarily to FIG. 234, the control system 15 may adjust a target flow rate (or mass flow rate) into each bag 26 as each bag 26 is filled. The flow rate may be adjusted by altering a duty cycle or position command to the dispensing flow path flow control valve 4048. The control system 15 may generate the command for this valve 4048 based on the output of a P, PI, or PID loop informed by a target dispensing fluid flow rate and the flow rate sensed by the flow rate sensors 4050A, B. A flowchart 7200 depicting a number of example actions which may be executed to transfer a target amount of fluid into a bag 26 is shown in FIG. 234. As shown, in block 7202, the target flow rate (which may be a mass flow rate) of fluid into the bag 26 may be ramped from zero to a predefined first target flow rate. This may be done at a predetermined ramp rate in various embodiments. Ramping of the target flow rate may help to allow time for other control loops governing the fluid handling system 3900 to adapt to the outgoing flow. This may assist in limiting overall control complexity for the fluid handling system 3900.

After the flow rate has been ramped to the first predefined target, fluid may be transferred into the bag 26 at this flow rate in block 7204. Once, in block 7206, the fill level of the bag 26 reaches a first fill target (e.g. a predefined mass) the target flow rate may be ramped to a second predefined target flow rate in block 7208. The flow rate may be ramped to the second predefined target flow rate at a second predetermined ramp rate (or the same ramp rate in block 7202 may be used). The second predefined target flow rate may be a flow rate less than the first predefined target flow rate. In some examples, for a one liter bag 26, the first predefined target flow rate may be at least 0.4 Lpm and the second predefined target flow rate may be no more than 0.2 Lpm (e.g. 0.1 Lpm). Alternatively, the first predefined target flow rate may for example be 30-50% (e.g. 40%) of the capacity of the bag 26 being filled per minute. The second predefined target flow rate may, for example, be 5-15% (e.g. 10%) of the capacity of the bag 26 being filled per minute. Adjustment of the target flow rate to the second predefined target flow rate may assist in precisely achieving the overall target fill level for the bag 26. The first fill target for the bag 26 may be set as a value equal to the overall target fill level less a predefined reserve amount.

Fluid may be transferred into the bag 26 at the second predefined target flow rate in block 7210. Once, in block 7212, the fill level of the bag 26 reaches a second fill target (e.g. a predefined mass) the closure of the flow control valve 4048 may be commanded by the control system 15. The second fill target may all be referred to as a pre-activation target and may be set as a value equal to the overall target fill level less an amount of fluid anticipated to pass through the flow control valve 4048 as it actuates closed.

If, in block 7216, all acceptance criteria imposed on filling of the bag 26 have been conformed with, the control system 15 may indicate the outcome of the fill operation to be a success or pass in block 7218. Otherwise, the control system may indicate that the fill outcome is a failure in block 7220. This determination and various supporting data may be documented in the batch record for the particular bag 26 (see, e.g., Table 3 & 4). The data included may be collected from the point the bag 26 is placed at a filling station 2600 until the bag 26 has flow control valve 4048 has closed.

The fill targets, target flow rates, and ramp rates may differ depending on the capacity of the bag 26 being filled. For example, lower values may be used for smaller bags 26. There may be specific targets and ramp rates predefined for each bag 26 type the system is compatible with. Specific settings for 50 ml, 100 ml, 250 ml, 500 ml, and 1000 ml bags 26 may be predefined and used by the control system 15 as appropriate. Settings for larger bag 26 sizes may also be defined in certain implementations.

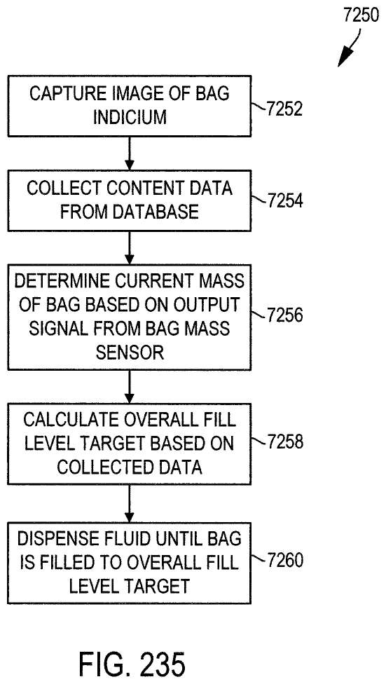

Referring now to FIG. 235, in some examples, the overall fill target for each bag 26 may be calculated by the control system 15. This may help to achieve desired concentration tolerance ranges for a particular end solution generated in the bag 26. The overall fill target may be calculated for any concentrate containing bag 26. A flowchart 7250 detailing a number of example actions which may be executed to dispense fluid into a bag 26 with a calculated overall fill target is shown in FIG. 235.

As shown in FIG. 235, an image may be captured of an indicium 56 (see, e.g., FIG. 7A) on a bag 26 in block 7252. This image may, for example, be collected from a chain orientation sensor 6730 where the chain orientation sensor 6730 is an imager. A dedicated imager for this purpose may also be used (e.g. positioned opposite the fill station 2600). Content information relating to the bag 26 may be collected from a database 6970 in block 7254. For example, the indicium 56 may include a unique identifier for the bag 26. During manufacture, various information about concentrate loaded into the bag 26 may be measured. This information may be associated with the unique identifier for the bag 26 and provided to the database 6970 by the manufacturer. This information may be used by the control system 15 to determine an overall fill level bespoke to a given bag 26 in order to achieve a target concentration for the desired solution to be produced in the bag 26. Bag content information may include one or more of the mass of the bag 26 before loading with concentrate, the mass of the bag 26 after loading with concentrate, the mass of concentrate dispensed to the bag 26, and the molarity of a brine dispensed into a bag 26. In some examples, the overall target fill amount may be determined by the manufacturer and associated with the unique identifier for each bag 26 in the database 6970 as well. Where the overall target fill amount is collected from the database 6970, the flowchart 7250 may proceed to block 7260 after block 7254. The control system 15 may orchestrate filling of the bag 26 based on the overall target fill amount defined in the database 6970.

Optionally, in certain examples, the bag 26 may be massed just prior to filling. Where such mass information is collected, the current mass of the bag 26 may be determined by the control system 15 based on the output of a bag mass sensor in block 7256. As mentioned elsewhere herein, the bag mass sensor may be a load cell or strain gauge included in the fill displacement stage 3142. This may be optional or may only be done for certain concentrates. For example, where the concentrate is a brine or includes a liquid component, the current mass of the bag 26 may be determined. This may allow the overall fill level target for a bag 26 to account for any evaporation of solvent through the material forming the bag 26 during storage.

In block 7258, the control system 15 may calculate an overall fill level target for the bag 26. Filling of the bag 26 may then be orchestrated in block 7260 (further described in relation to FIG. 234). Where the current mass of the bag 26 is not collected, the overall fill level target may be calculated based on the mass of concentrate documented in the database 6970 as being in the bag 26 after manufacture. The mass and molarity of a brine or liquid concentrate in the bag 26 may also be used to calculate an overall target fill level. Where the current mass of the bag 26 is determined, the current mass may be compared to content information collected from the database 6970. Using the example of a brine filled bag 26, any loss of solvent (e.g. water) in the bag 26 due to evaporation through the bag 26 material during shipping/storage may be determined. This loss may be added to the overall target fill level which would have been calculated from the content information in the database 6970. The determined solvent loss may also be utilized by the control system 15 to select the motion profile used by a mix assisting assembly 2500. If the current mass of the bag 26 differs from the bag mass stored in the database 6970 by more than a threshold, the bag 26 may be flagged for rejection. In some examples, the bag 26 may not be filled and may be displaced through the system 10 to a receptacle 4120B in an outfeed assembly 4100 for rejected bags 26.

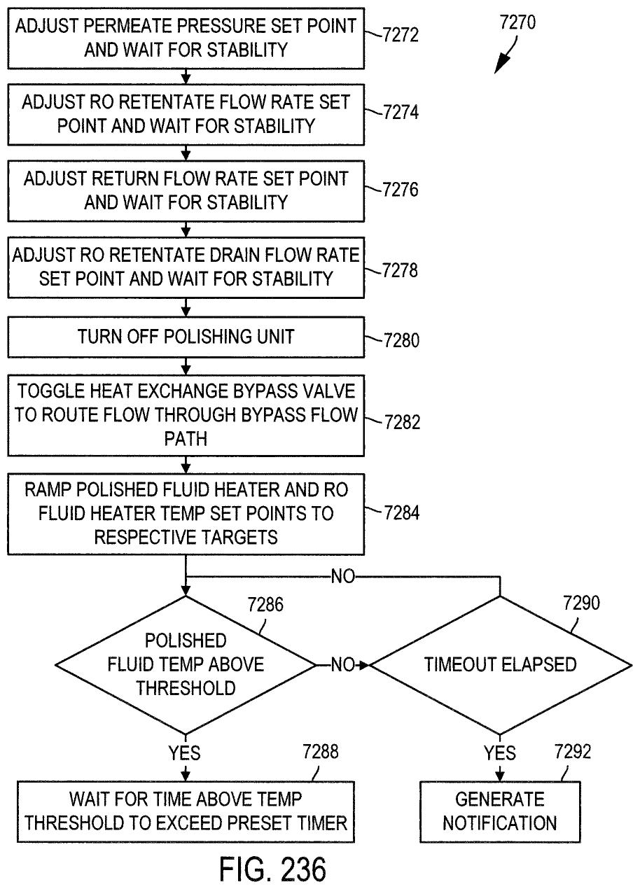

Referring now to FIG. 236, a flowchart 7270 detailing a number of example actions which may be executed to generate and circulate a disinfect fluid in a fluid handling system 3900 is depicted. The example flowchart 7270 begins with the fluid handling system 3900 producing fluid for filling of reservoirs. As shown, in block 7272, the permeate pressure set point target 7054 for the filter inlet pump 3954 control loop may be adjusted (e.g. decreased). Ramping may be employed to limit the rate of adjustment for all set point changes. The control system 15 may also, in block 7272, monitor the permeate pressure sensor 3990 and wait until the sensed permeate pressure is within a range of the target for a period of time. Once the permeate pressure is stable, the flow rate set point for fluid in the recirculation flow path 3972 may be adjusted (e.g. decreased) in block 7274. After this flow rate is stable within a range of its target, the flow rate set point in the return flow path 4028 may be adjusted in block 7276. Once the flow rate in the return flow path is stable within a range of its target, the retentate drain flow rate may be adjusted (e.g. decreased) in block 7278. The control system 15 may also monitor the retentate drain flow rate and ensure it is stable within a range of its target. In the event that the control system 15 determines that any of the process variables fail to stabilize after set point adjustment, the control system 15 may generate a notification and the system 10 may be placed in a standby state. The set point adjustments may generally lower flow rates within the fluid handling system 3900 and limit the amount of fluid exiting the fluid handling system 3900.

Still referring to FIG. 236, once the fluid handling system 3900 has stabilized at the adjusted set points, the polishing unit 3988 may be turned off in block 7280. In block 7282, the control system 15 may command the heat exchange bypass valve 3938 to toggle to a bypass position. This may route fluid entering the accumulator 3934 around the heat exchanger 3936 and into the bypass flow path 3940. Any irradiators 3956, 4010 may be switched off in block 7282 as well. The set points for filter assembly heater assembly 3966 and the polished fluid heater 4016 may be ramped to respective disinfect set points in block 7284. The disinfect set points for each heater 3966, 4016 may be 80-90° C. or higher in certain examples. The accumulator heater 3948 may typically be controlled to a high temperature set point even during the production of fluid for filling of bags 26. That said, in some embodiments, the accumulator heater 3948 set point may also be ramped to a disinfect set point (e.g. from 80° C. to 90° C.) in block 7248.

If, in block 7286, the polished fluid output temperature sensor 4024 indicates the fluid temperature is below a threshold (e.g. 65° C.) and if a timeout has elapsed in block 7290, the control system 15 may generate a notification in block 7292. The system 10 may also be placed in a standby state by the control system 15. Once, in block 7286, the polished fluid output temperature sensor 4024 indicates the fluid temperature is above the threshold, the control system 15 may wait for a disinfect timer to elapse in block 7288. The timer may, for example, require the temperature be above the threshold for 1 hour (though this time would be dependent on the threshold). Disinfection may be determined to have completed when the disinfect timer has elapsed.

In some embodiments, the control system 15 may monitor for the fluid temperature to breach a first threshold (e.g. 65° C.). The disinfect timer may, however, only be incremented after the temperature breaches a second, higher threshold (e.g. 70° C. or more). This may allow for easier differentiation as to whether the fluid handling system 3900 failed to heat up or failed to complete a heat soak.

In some embodiments, the data signal from the polished fluid output temperature sensor 4024 may not necessarily be used. The control system 15 may, for example, check all temperature sensors downstream of the accumulator 3934 and ensure the lowest reported value is above the threshold. All temperature values indicated by these temperature sensors may be required to be above the threshold before incrementing the disinfect timer.

The disinfect timer may only increment when the data indicates fluid in the fluid handling system 3900 is above the disinfect temperature threshold. In some embodiments, an excursion below the disinfect temperature threshold may cause the disinfect timer to reset. The control system 15 may enforce a timeout which causes a notification to be generated in the event that the disinfect timer does not fully elapse within a predetermined time window (e.g. 3 hours).

In some examples, the disinfect process may use a disinfection fluid other than heated water or have the option of using a disinfection fluid other than heated water. For example, in some embodiments, a reservoir of disinfection chemical may be plumbed into fluid communication with the fluid handling system 3900. An oxidizing agent such as peracetic acid solution may be used in some implementations. A volume of disinfection chemical may be dispensed (e.g. pumped) into the fluid handling system 3900 to create a desired concertation within the fluid handling system 3900. Where peracetic acid is used, the target concentration may be 1%. Where a disinfection chemical is used, a flush of the fluid handling system 3900 may be conducted after disinfection is completed.

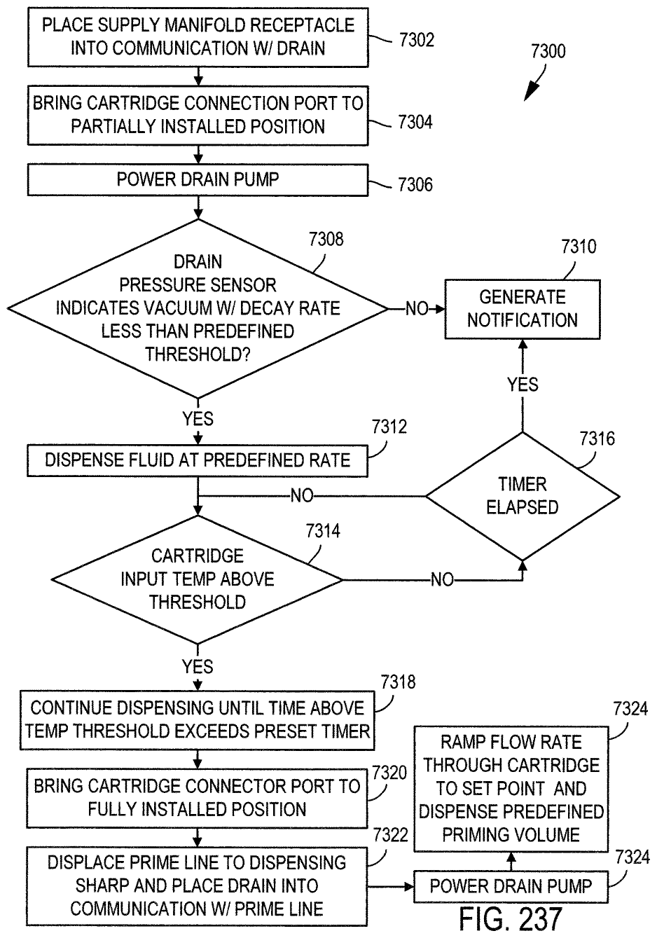

Referring now to FIG. 237, flowchart 7300 is depicted detailing a number of example actions which may be executed to install and prime a cartridge 2606 at a fill station 2600. The example flowchart 7300 begins with the fluid handling system 3900 producing hot water (see, e.g., FIG. 236). In block 7302, the supply manifold 2820 may be placed into fluid communication with a drain 3930H. The control system 15 may for example, command toggling of the flow selector valve 4058 to establish a flow path from the disinfect outlet flow path 2824 to the drain flow path 4060. In block 7304, the cartridge connection port 2812 may be brought to a partially installed position within the receptacle 2818 of the supply manifold 2820 (further described in relation to FIG. 126A). A drain pump 4064 may be powered in block 7306. If, in block 7308, the data signal from the drain pressure sensor 4062 indicates a vacuum has been established and decays at less than a predefined rate, the control system 15 may command hot fluid be dispensed to the supply manifold 2820 in block 7312. If, in block 7308, an acceptable vacuum is not established, a notification may be generated in block 7310.

Once dispensing of fluid to the supply manifold 2820 begins, the control system 15 may monitor the data signal from the cartridge input temperature sensor 4054. If, in block 7314, the temperature is below a threshold (e.g. 65° C.) and a warm up timer has elapsed in block 7316, a notification may be generated in block 7310. If, in block 7314, the temperature indicated by the cartridge input temperature sensor 4054 is above the threshold, the control system 15 may continue dispensing fluid to the supply manifold 2820 in block 7318. Fluid may continue to be dispensed in block 7318 until the data signal from the cartridge input temperature sensor 4054 indicates the temperatures has been above the threshold for more than a predefined period of time. The connection port 2812 of the cartridge 2606 may be brought to a fully installed position (further described in relation to FIG. 126B) in block 7320. This may drive the spike 2822 through the frangible 2816 of the cartridge 2606 and place the interior of the cartridge 2606 into fluid communication with the fluid handling system 3900.

In block 7322, the prime line 3120 may be displaced to the dispensing sharp 2604 of the cartridge 2606. The prime line 3120 may also be placed into fluid communication with a drain 3930H in block 7322. The flow selector valve 4058 may, for example, be toggled by the control system 15 to place the prime line 3120 into communication with the drain 3930H. As further described in relation to FIG. 127, an irradiation assembly 2608 may be powered after the prime line 3120 is in position. In block 7324, the drain pump 4064 may be powered. In block 7324, flow through the cartridge 2606 may be ramped to a set point and fluid may be dispensed until a predefined priming volume has been transferred through the cartridge 2606. The ramp rate used may be selected to inhibit shock to the filter 2609. The ramp rate may also be selected to ensure gas is driven to the downstream side of the filter 2609 before the filter 2609 is fully wetted due to wicking of priming fluid.

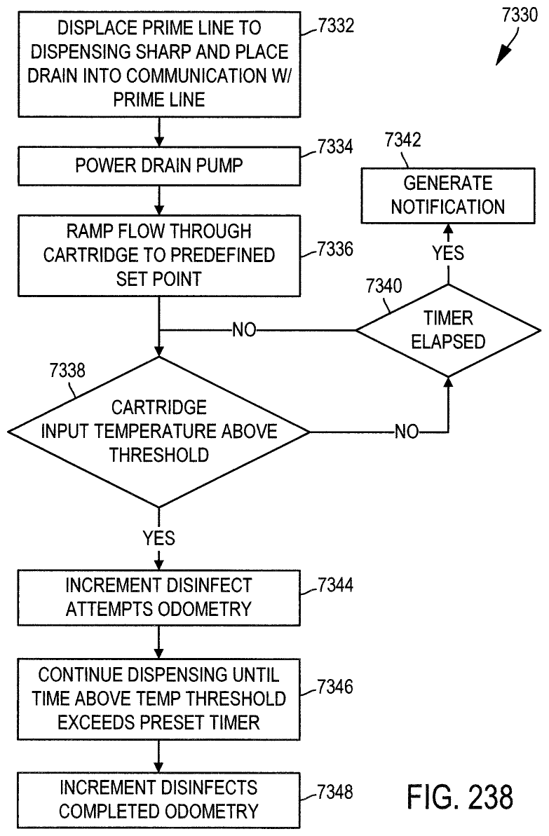

Referring now to FIG. 238, flowchart 7330 is depicted detailing a number of example actions which may be executed to disinfect a cartridge 2606 installed in a fill station 2600. The flowchart 7330 begins with the fluid handling system 3900 producing disinfect fluid (further described in relation to FIG. 236). As shown, the prime line 3120 may be displaced to the dispensing sharp 2604 in block 7332. The prime line 3120 may also be placed in fluid communication with a drain 3930H in block 7332. The control system 15 may, for example, send a command to the flow selector valve 4058. The drain pump 4064 may be powered in block 7334. Fluid may be provided to the cartridge 2606 through the supply manifold 2820 in block 7336. The flow rate may also be ramped to a predefined set point in block 7336. The control system 15 may monitor the data signal from the cartridge input temperature sensor 4054 to determine if the temperature of fluid provided to the cartridge 2606 is above a threshold. If, in block 7338, the cartridge input temperature is below the threshold and a timer has elapsed in block 7340, a notification may be generated in block 7342. The system 10 may also be placed in a standby state. If, in block 7338 the cartridge input temperature breaches the threshold a cartridge disinfect attempt odometry counter may be incremented in block 7344. In block 7346, fluid may continue to be dispensed through the cartridge 2606 until the temperature has been above the threshold for at least a predefined period of time. After fluid above the temperature threshold has been transferred through the cartridge 2606 for the predefined period of time, a cartridge disinfect may be deemed to have successfully completed by the control system 15. A cartridge disinfect odometry counter may be incremented by the control system 15 in block 7348.

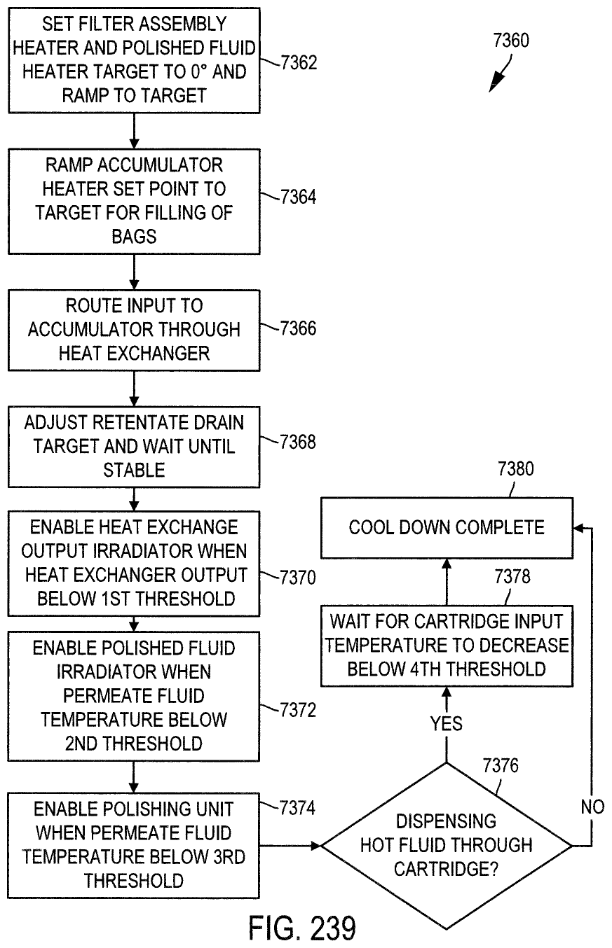

Referring now to FIG. 239, flowchart 7360 is depicted detailing a number of example actions which may be executed to actively cool a fluid handling system 3900 after a completing a heat disinfect. Typically, after a heat disinfect, a fluid handling system 3900 may be permitted to rest as it naturally cools down. That said, in for example, a scenario with unexpected demand, it may be desired that a fluid handling system 3900 more quickly transition to a state in which it is ready to produce filled bags 26 or other reservoirs for consumption. In such an instance, a user may command (e.g. via input to a user interface 6100) the fluid handling system 3900 to actively cool itself to a temperature appropriate for production of reservoirs.

The flowchart 7330 begins with the fluid handling system 3900 producing disinfect fluid (further described in relation to FIG. 236). As shown, in block 7362, the temperature targets for the filter assembly heater 3966 and the polished fluid heater 4016 may be ramped to a low temperature set point. The set point in block 7362 is listed as 0° C., however, any set point where these heaters would not need to be powered may be used. The accumulator heater 3948 temperature set point may also be ramped to a temperature set point target for filling of bags 26 in block 7364. Fluid input to the accumulator 3934 may be routed through the heat exchanger 3936 in block 7366. In block 7368, the retentate drain rate target may be adjusted. This target may significantly higher (e.g. 3-4 or more times) the drain rate target used while the fluid handling system 3900 is producing heated disinfect fluid. This may assist in quickly removing heat from the fluid handling system 3900. The control system 15 may also wait for the retentate drain rate indicated by the retentate drain flow sensor 3976 to be within a range of the target for a predefined period of time in block 7368. In block 7370, the heat exchanger outlet irradiator 3956 may be enabled when the control system 15 detects the output signal from the heat exchanger outlet temperature sensor 3957 is below a first threshold. The polished fluid irradiator 4010 may be enabled when the control system 15 detects the output signal from the permeate fluid temperature sensor 3994 is below a second threshold in block 7372. The polishing unit may be enabled by the control system 15 in block 7374 once the output signal from the permeate fluid temperature sensor 3994 is below a third threshold.

If, in block 7376, the fluid handling system 3900 is not dispensing hot fluid from the cartridge 2606, the cool down may be deemed complete in block 7380 when the polishing unit is enabled. If, in block 7376, the fluid handling system 3900 is dispensing heated fluid, fluid may continue to be dispensed from the cartridge 2606. The control system 15 may wait, in block 7378, for the output signal from the cartridge input temperature sensor 4054 to indicate the temperature of fluid passing to the cartridge 2606 is below a fourth threshold. The cool down may then be determined to be complete in block 7389.

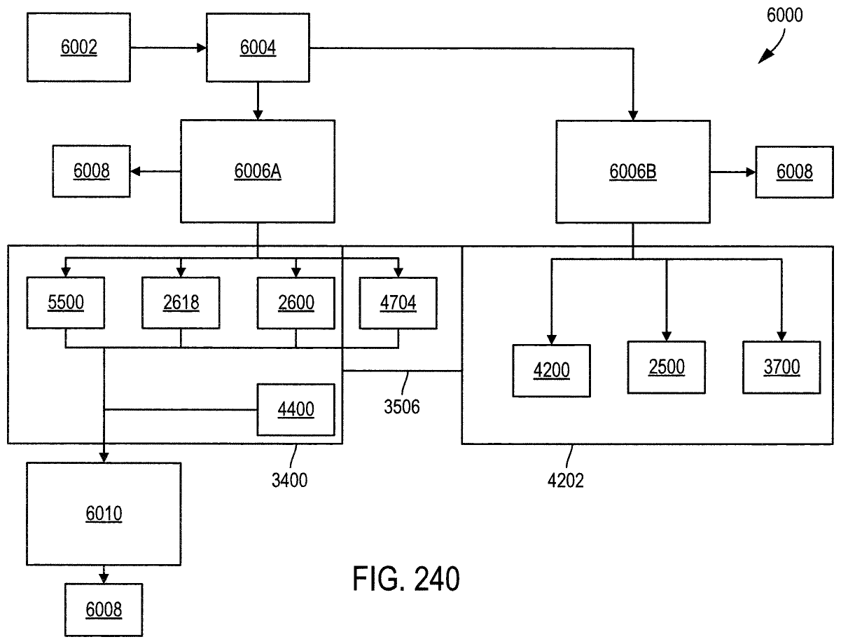

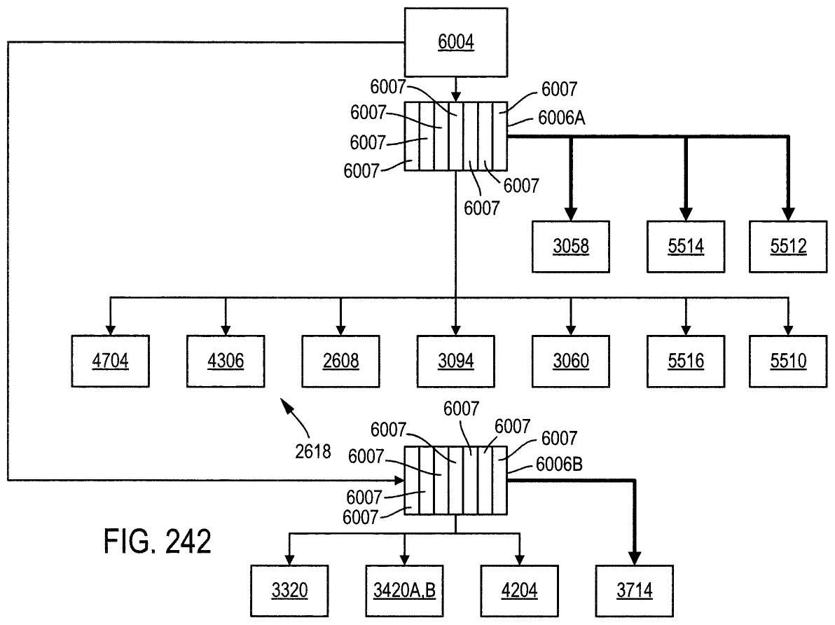

Referring now to FIG. 240, a block diagram of an example pneumatic distribution system 6000 which may be included in the systems 10 described herein is depicted. The pneumatic distribution system 6000 may receive air from a pressurized air source 6002. The pressurized air source 6002 may be a shop air system or other compressed air utility. The pressurized air source 6002 may provide pressurized air at a pressure between 80-120 psi in certain examples. Air from the pressurized air source 6002 may pass through a preparation assembly 6004 which may include various filters, driers, pressure sensor, and pressure regulators. Air from the preparation assembly 6004 may be plumbed to one or more valve bank 6006A, B. In some examples, and as depicted, a first valve bank 6006A may be included for controlling supply of air to components in the processing chamber 4300 (see, e.g., FIG. 2). A second valve bank 6006B may be included for provisioning air to other components (e.g. those in the outfeed compartment 4202). Each valve bank 6006A, B may include a plurality of pneumatic valves 6007 (see, e.g., FIG. 242) of the same or differing varieties. The valve banks 6006A, B may receive pressurized air regulated to a plurality of different pressures from the preparation assembly 6004.

Still referring to FIG. 240, the first valve bank 6006A may supply pressurized air to the sanitary interface assembly 5500, the bag displacement assembly 2618, and the fill station 2600. Where the splitter assembly 4406 of the bag individualization assembly 4400 is pneumatically driven, the bag individualization assembly 4400 may also receive pressurized air via the first valve bank 6006A. In certain examples, the first valve bank 6006A may further supply pressurized air to any components in the transfer chamber 3506. Where a pneumatic gripper is used in the bag retainer 4704 of the transfer chamber 3506, air to actuate the gripper may be supplied via the first valve bank 6006A. The second valve bank 6006B may supply pressurized air to the gantry assembly 4200, mix assisting assembly 2500, and marking assembly 3700.

A vacuum source 6010 may also be included in the pneumatic distribution system 6000. The vacuum source 6010 may, for example, be a vacuum pump. The vacuum source 6010 may be in communication with sanitary interface assembly 5500, the bag displacement assembly 2618, the fill station 2600, and the bag individualization assembly 4400. The vacuum source 6010 may also be plumbed into communication with the bag retainer 4704 of in the transfer chamber 3506. The negative pressure supplied to these components may be leveraged to draw particulate generated by these component out of the processing chamber 4300 or transfer chamber 3506. The valve banks 6006A, B and vacuum source 6010 may also be in communication with an exhaust 6008.

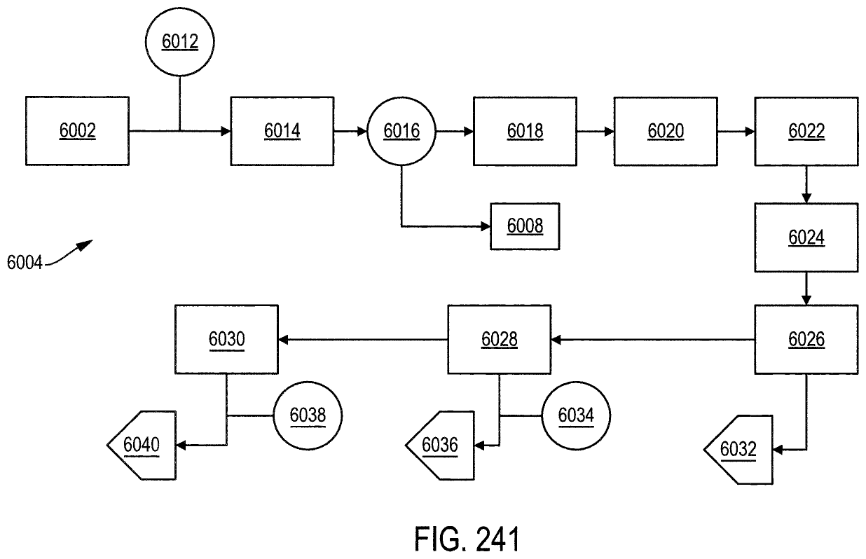

Referring now to FIG. 241, a block diagram of an example preparation assembly 6004 is depicted. The preparation assembly 6004 receives air from a pressurized air source 6002. The pressure of the air supplied from the pressurized air source 6002 may be monitored by at least one pressure sensor 6012. In the event that the pressure of air from the air source 6002 is not within a predefined range, a valve 6016 of the preparation assembly 6004 may be closed. A pressure regulator 6014 may be included to regulate the pressure of incoming air to a range or set point. The range or set point may be selected based upon pressure tolerance of downstream filters or other components.

After passing the pressure regulator 6014, air may be passed through a first filter 6018 and a second filter 6020. The first and second filters 6018, 6020 may remove particulate from the air supplied to the pneumatic distribution system 6000. The first filter 6018 may be a coarser filter than the second filter 6020. In some examples, the first filter 6018 may be a 1 micron filter and the second filter may be a 10 nm filter. The filtered air may then pass to a drier 6022 which may remove moisture content from the air. The drier 6022 may be a membrane type drier in certain embodiments. An additional filter 6024 (e.g. 10 nm) may be disposed downstream of the dryer 6022.

Dry filtered air may then be passed to a set of output regulators 6026, 6028, 6034. A greater or lesser number of output regulators 6026, 6028, 6034 may be included depending on the pressures desired to be supplied throughout the system 10. The first output regulator 6026 may provide pressurized air to a set of pilot air flow paths 6032 plumbed to the valve banks 6006A, B. The first output regulator 6026 may regulate air to a set point of 75-85 psi (e.g. 80 psi) in certain examples. In embodiments where the valve types used in the valve banks 6006A, B do not call for a pilot air source, the first output regulator 6026 and pilot air flow paths 6032 may be omitted. The second output regulator 6028 and third output regulator 6030 respectively provide high and low pressure air to various components in the system 10. The second output regulator 6028 may, for example, output 75-85 psi air to a set of high pressure flow paths 6036 to the valve banks 6006A, B. A pressure sensor 6034 may be included to monitor the pressure in the high pressure flow paths 6036. The second pressure regulator 6028 may be a soft start type regulator. The second pressure regulator 6028 may slowly ramp up the pressure in the high pressure flow paths 6036 to the high pressure set point. This may ensure that the pilot flow paths 6032 quickly attain their pressure set point while ensuring a lag is present in the high pressure flow paths 6032. The third output regulator 6030 may provide air at a pressure of 45-55 psi to a set of low pressure flow paths 6040 extending to the valve banks 6006A, B. A pressure sensor 6038 may be included to monitor pressure in the low pressure flow paths 6040. The control system 15 may monitor the output signal from the pressure sensors 6034, 6038 and may generate a fault in the event that the sensed pressure is in breach of a predefined expected range. The control system 15 may place the valves 6007 (see, e.g., FIG. 242) of the valve banks 6006A, B in a default state in the event an unexpected pressure is sensed.

Referring now to FIG. 242, different pressures may be supplied to different components of the system 10 via the valve banks 6006A, B. As shown, low pressure air may be supplied, via the first valve bank 6006A to the bag retainer 4704 in the transfer chamber 3506 (where the bag retainer 4704 includes a pneumatically actuated gripper). Low pressure air may also be supplied to the bag retention assemblies 4306 of the bag displacement assembly 2618. This air may be selectively supplied (by toggling valves 6007 in the valve bank 6006A) to the bag retention assemblies 4306 to actuator the jaws 4308A, B (see, e.g., FIG. 68A) of the bag retention assemblies 4306. Low pressure air may also be supplied to the irradiation assembly 2608 of the fill station 2600. This air may be selectively communicated to the actuation assembly 3139 (see, e.g., FIG. 131) in embodiments where the irradiation assembly 2608 is pneumatically driven through its displacement range. Low pressure air may also be selectively provided to the clean air output actuation assembly 3094 to displace the coupled displacement stage 3099 (see, e.g., FIG. 116). This may in turn engender a displacement of the clean air output 3090 (see, e.g., FIG. 118) to raise and lower the clean air output 3090 relative to an installed cartridge 2606. The cover engagement assembly 3060 (further described in relation to FIG. 108B) may also selectively receive low pressure air via the valve bank 6006A. A door rotation brake 5516 and a door latch 5510 of the sanitary interface assembly 5500 may additionally selectively receive low pressure air via the valve bank 6006A.

High pressure air may be supplied, via toggling of valves 6007 in the valve bank 6006A, to a cover displacement assembly 3058 of the cover removal assembly 3050. High pressure air may also be provisioned, via the valve bank 6006A to rotational and translational door displacement actuators 5512, 5514 of a sanitary interface assembly 5500.

The second valve bank 6006B may similarly supply air at a variety of pressure to components of the system 10. High pressure air may selectively be supplied to an embossing actuator 3714 of a marking assembly 3700 through the valve bank 6006B. Low pressure air may be selective passed to the gripper assembly 4204 of the gantry assembly 4200 as well as the gripper 3320 of the bag retention assembly 3310 of the mix assisting assembly 2500. The door actuators 3420A, B of the bag retention assembly 3310 may also selective receive low pressure air via the valve bank 6006B.

The valves 6007 of the valve banks 6006A, B may be selected based on the component they are paired with. In the event of a loss of electrical control, the valves 6007 may transition to a default state which places the component in a desired state.

Valves 6007 gating pneumatic pressure to bag holding components may be arranged to default to a valve state which supplies pressure that maintains the component in a holding state. Bag holding components, may include the jaws 4308A, B of the bag retention assemblies 4306, the jaws 4205A, B of the gantry assembly 4200, the gripper 3320 of the mix assisting assembly 2500, and the door actuators 3420A,B for each door 3400A, B of the mix assisting assembly 2500. Any other pneumatic grippers that may be included in the system 10 may similarly be paired with such a valve. Single solenoid 5/2 valves with a normally open or closed position may for example be used.

Any components which have a position which is always non-interfering with other components may be paired with a valve which defaults to a state in which the component is driven to non-interfering position. The clean air output actuation assembly 3094 may for example be paired with a valve 6007 which drives the clean air output 3090 to a raised position. Similarly components with an otherwise known desired default position may be paired with a valve 6007 that supplies pressure to drive the component to that position by default. The embossing actuator 3714 may for example be paired with a valve 6007 that supplies pressure to displace the 3702 to a retracted position by default. Single solenoid 5/2 valves with a normally open or closed position may for example be used.

Any components that position along a horizontal axis may be pair with valves 6007 that permit the components to stay in place when in their default state. Preferably, such components may be manually moved by service personnel. Center exhaust 5/3 valves may for example be used. Components which displace along a non-horizontal axis (e.g. vertical) may be paired with valves 6007 the hold them in place against gravity, but do not move them. The actuation assembly 3139 (see, e.g., FIG. 131) for the irradiation assembly 2608 may be paired with such a valve 6007. Center pressure 5/3 valves may for example be used.

Referring now to FIG. 243, a block diagram of an example negative pressure distribution assembly 6060 of the pneumatic distribution system 6000 is depicted. The example negative pressure distribution assembly 6060 includes a vacuum source 6010 which in the example embodiment is a pump. The vacuum source 6010 may include a temperature sensor 6044 which outputs a signal which varies in relation to the temperature of the vacuum source 6010. The control system 15 may generated a fault if the temperature indicated by the temperature sensor 6044 is outside of a predefined expected range. A muffling assembly 6041 may be included to mitigate noise generated by the vacuum source 6010. The muffling assembly 6041 may include an inline silencer and a vent silencer in series in certain examples. A condensate drain 6042 may be displaced intermediate the muffling assembly 6041 and the vacuum source 6010 in certain embodiments. Between the vacuum source 6010 and one or more pressure distribution manifold 6054 may be a relief valve 6046. The relief valve 6046 may be selected to open when a pressure in excess a predetermined limit is present. A valve 6048 may also be included intermediate the vacuum source 6010 and the at least one manifold 6054. The valve 6048 may be toggled to place the pressure source 6010 into communication with ambient 6050 if desired. A filter 6052 may be included between the valve 6048 and the manifold(s) 6054. The filter 6052 may capture any particulate which is evacuated from the system 10 via the negative pressure distribution assembly 6056. A pressure sensor 6056 may be disposed intermediate the filter 6052 and the manifold(s) 6052. The control system 15 may monitor the signal from the pressure sensor 6056 to confirm vacuum is being provided to the manifold(s) 6054 by the vacuum source 6010. This may allow the control system 15 to detect a fouled filter 6052 for example. A fault may be generated by the control system 15 if the data signal from the pressure sensor 6056 indicates a pressure of at least some predetermine magnitude cannot be maintained.

As shown, negative pressure may be supplied through the manifold(s) 6054 to a variety of components in the system 10. Vacuum may be supplied for a variety of reasons. Vacuum may be supplied to evacuate potential particulate generated by components from the processing compartment 4300, transfer chamber 3506, and optionally the outfeed compartment 4202. Vacuum may be supplied adjacent bearing and guide interfaces, in housings for motors or actuators, and into communication with vacuum ports on pneumatic displacement stages for removal of particulate. Vacuum may be supplied to certain components in order to assist in maintaining a controlled environment within the enclosure 12. Vacuum may be supplied to detect potential breaches of the controlled environment as well. The manifold(s) 6054 may include needle valves which may be adjusted to regulate the flow to each component. Alternatively, binary type solenoid valves may be included and may be operated based on a PWM signal provided from the control system 15 to regulate the pressure. Any other suitable valving arrangement such as variable orifice valves may be used.

One or more vacuum conduit 6058 may extend to the bag displacement assembly 2618. For example a vacuum conduit 6058 may be placed into communication with interior of the actuator housing 4328 (see e.g., FIG. 68B) for the extension actuator assembly 4330 for each bag retainer assembly 4306. Vacuum may also be supplied to the bag retention assemblies 4306 proximate the interface between the bearings 4325 and jaw bearing guide 4323 (see, e.g., FIG. 69A and FIG. 72A). A vacuum conduit may be routed to the interior of the housing 4728 for the rotary actuator of the bag retainer 4704 in the transfer chamber 3506. Where the bag retainer 4704 includes a gripper similar to those in example bag displacement assemblies 2618 described herein, vacuum may also be plumbed to any bearing interface for the gripper jaws. Vacuum may also be supplied to the cover engagement assembly 3060 in the vicinity of the bearing surfaces 3063 for the wing assemblies 3061. A vacuum conduit 6058 may also be plumbed to the bag displacement actuator 3144 for the bag displacement stage 3140 and the singulation actuator 4402 of the bag singulation assembly 4400. Vacuum may be supplied to various pneumatic displacement stages or actuators included within the system 10. For example, where the irradiation assembly 2608 is coupled to a pneumatic displacement stage, a vacuum conduit 6058 may be in communication with the actuation assembly 3139 (see, e.g., FIG. 131) for the irradiation assembly 2608. At least one vacuum conduit 6058 may also extend to the sanitary interface assembly 5500.

The bay 3054 of the housing 3052 (see, e.g., FIG. 108A) for the cover removal assembly 3050 (see, e.g., FIG. 108A) may be in selective communication with a vacuum conduit 6058 via a valve 6062. This may assist in retaining a cover 2996 (see, e.g., FIG. 113) within the bay 3054 of the housing 3052 when the cover engagement assembly 3060 (see, e.g., FIG. 108B) is in an engaged relationship with the cover 2996. One or more pressure sensor may be in communication with the bay 3054. The control system 15 may monitor the pressure indicated by the at least one pressure sensor 3051 to ensure a cover 2996 is sealed in place within the bay 3054 (described in greater detail in relation to FIG. 111B and FIG. 113). Thus, the vacuum source 6010 may assist in maintaining the controlled environment within the processing chamber 4300 and assist detection of a breach in the controlled environment.

Referring now to FIG. 244, a block diagram of an example air handling system 6200 which may be included in example systems 10 shown and described herein is depicted. The example air handling system 6200 may be particularly compact while supporting the establishment and maintenance of a number of different zones which may be considered separate or different from an environmental control perspective. The zones may be maintained at varying degrees of environmental control within the system 10. The air handling system 6200 may support transition between different levels of environmental control for a particular zone over time. The air handling system 6200 may also support merger of certain zones into the same environmental control regime and furcation of a zone into multiple zones controlled to differing levels of stringency.

Referring to now to FIGS. 244-246, the air handling system 6200 may receive air from within the enclosure 12 of the system 10 as well as ambient air 6202 surrounding the system 10. Preferably, the ambient air 6202 conforms to at least ISO 9 cleanroom standards. Ambient air 6202 (and air being recirculated within the enclosure 12) may pass to a pre-filter 6204. In various embodiments, the pre-filter 6204 is a coarse filter which removes large particles (e.g. 5 μm or larger) from incoming air. In various embodiments, the pre-filter 6204 may be have a minimum efficiency reporting value (MERV) of 11 or higher (e.g. 13). After passing through the pre-filter 4202, air may optionally be passed through at least one of a muffler 6206 and a drier 6208. Air may then be drawn to a blower 6210. The blower 6210 may be of the centrifugal variety in certain implementations.

A pressure sensor 6302 and a particulate sensor 6304 may be associated with the pre-filter 6204. Any suitable pressure sensors 6302 may be used. Particulate sensors described herein may be non-viable particulate counters (though viable counters such as bio-fluorescent particle counters may also be utilized in some air handling systems 6200). Particulate counters may be multi-channel type counters which output signals indicative of counts for particulate in respective size ranges. For example, particulate counters may output data on particulate in a first size range (e.g. 0.2-0.5 μm range) as well as counts for larger particulate (e.g. 5-30 μm size range). In some embodiments, counts for particulates in several (e.g. four-five) different size ranges may be output by the particulate sensors. For example, the pressure sensor 6302 and particulate sensor 6304 may be disposed downstream of the pre-filter 6204. The pressure from the pressure sensor 6302 may be compared to an output signal from an enclosure pressure sensor 6336 by the control system 15. In the event the control system 15 determines a pressure drop greater than a predefined amount is present through the pre-filter 6204, the control system 15 may generate a fault. A fault may also be generated if a pressure drop is absent or less than an expected amount. In some embodiments, the control system 15 may collect data on the pressure drop over a prolonged period (e.g. hours, days) and a change in the observed pressure drop over time may be determined. In the event the pressure drop changes at greater than a predefined rate over time (potentially indicative of an issue at the host site) a fault may be generated. Operation of the system 10 may be prohibited until the pre-filter 6204 has been replaced or otherwise serviced in the event a fault is declared. The control system 15 may similarly generate a fault and inhibit further system 10 operation until service has been performed if data from the particulate sensor 6304 indicates particulate levels above a predefined threshold.

A humidity sensor 6300 may be included upstream of the drier 6208 in some examples. The control system 15 may monitor the data signal from the humidity sensor 6300 and may generate a fault in the event that the output from the humidity sensor 6300 indicates humidity has breached preset threshold (e.g. 70-80%). The control system 15 may also prevent operation of the system 10 in some examples. Any fault generated by the control system 15 may be accompanied by a notification generated by the control system 15 for display on a graphical user interface 6100 of the system 10. As shown, the blower 6210 may further include a humidity sensor 6306. The control system 15 may compare the output from the upstream humidity sensor 6300 and blower humidity sensor 6306. In the event that the output from each humidity sensor 6300, 6306 is suggestive of an issue with the drier 6208 (e.g. the drier 6208 decreases the humidity less than a predetermined expected amount), the control system 15 may generate a fault.

The blower 6210 may also include a flowrate sensor 6308, temperature sensor 6310, and pressure sensor 6312. In various examples, the control system 15 may issue a command to the blower 6210 to output air at a certain flowrate. The blower 6210 may include an on-board controller 6211 which may utilize data from the blower sensors 6306, 6308, 6310, 6312 to adjust blower speed to achieve the commanded flow rate. When the system 10 is producing filled bags 26, the flowrate for the blower 6210 may be set at 200-300 m³/hr (e.g. 250 m³/hr). When the system 10 is on standby and no orders for bags 26 are being processed, the blower 6210 may be run at a low power setting. The flowrate at the lower power setting may be 75-150 m³/hr. The flowrate selected for the low power setting may be sufficient to ensure at least 0.2 m/s air velocities in at least the processing chamber 4300. The blower 6210 may also be shut off during certain scenarios. For example, the blower 6210 may not be powered when certain maintenance procedures are being performed (e.g. during a vaporized hydrogen peroxide cycle). In still other scenarios, the blower 6210 may be set to run in a high flowrate mode. For example, the flowrate may be set at at least 400-450 m³/hr in a service mode to allow for a panel (see, e.g., panel 7601 of FIG. 141) to be removed from the processing chamber 4300. This may, for instance, allow a gowned individual access for maintenance or cleaning. The flowrate selected may be sufficient for air to pass from the processing chamber 4300 through the opening previously covered by the panel at a velocity of at least 2.5 m/s (e.g. 3-4 m/s).

Still referring to FIGS. 244-246, air from the blower 6210 may be directed into a guide passage 6212 leading to a flow straightener 6214. The walls of the guide passage 6212 may be angled to mitigate any turbulence in the airflow leaving the blower 6210 as the air is displaced through the guide passage 6212 to the flow straightener 6214. The flow straightener 6214 may assist in increasing the laminar character of the airflow out of the blower 6120. In certain embodiments, the flow straighten 6214 may include an array of elongate channels 6260 (see, e.g., FIG. 246). The channels 6260 may, for instance, have rectangular or square cross-sections. The cross-sectional area of each of the channels 6260 may be substantially the same. The air may then pass through a first routing chamber 6216 and toward a first laminarizer 6218. The first routing chamber 6216 may form a bend in the airflow path. In the example shown in FIG. 246, a substantially 90° bend is imposed by the first routing chamber 6216. The first laminarizer 6218 may also be an array of elongate channels 6262 (see, e.g., FIG. 246). The channels 6262 may have a round or circular cross-section in various examples. The cross-sectional area of each of the channels 6262 may be substantially the same and smaller than the cross-sectional area of the channels 6260 in the flow straightener 6214. As air passes through the first laminarizer 6218, air may exit into a second routing chamber 6220. The second routing chamber 6220 may form a bend in the airflow path. As best shown in FIG. 246, a substantially 90° bend is created by the second routing chamber 6220. A second laminarizer 6222 may be placed at the downstream end of the second routing chamber 6220. The second laminarizer 6222 may also include an array of elongate channels 6264 extending therethrough. The channels 6264 may have a round or circular cross-section in various examples. The cross-sectional area of each of the channels 6264 may be substantially the same and smaller than the cross-sectional area of the channels 6262 in the first laminarizer 6218.

Though the air flowing through the air handling system 6200 may have a relatively high Reynolds number (e.g. ~10000), the serial placement of the flow straightener 6214 and laminarizers 6218, 6222 prevents the turbulent kinetic energy within the flow from increasing in an unchecked manner. Each of the flow straightener 6214 and laminarizers 6218, 6222 may act as a turbulent kinetic energy dampener which reduces the turbulent kinetic energy of the airflow. As a result, the airflow may possess a high Reynolds number, but behave in a generally laminar manner and be referred to as laminar or laminarized herein. Though three turbulent kinetic energy dampeners are shown, a greater or lesser number may be included in alternative embodiments. Care may also be taken to ensure no stagnant zones are present within the series of alternating kinetic energy dampeners and routing chambers 6216, 6220.

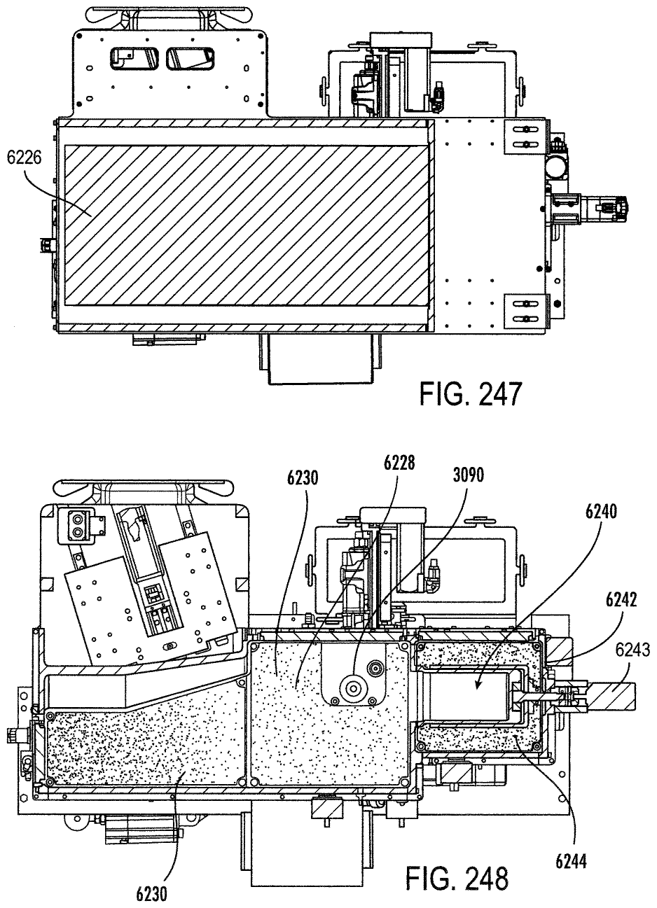

Referring now to FIGS. 244-245 and FIG. 247, air may pass through the second laminarizer 6222 to a filtration chamber 6224 in which a filter 6226 is housed. The filter 6226 may capture particulate of 0.2 μm or smaller (e.g. 0.12 μm). In certain examples, the filter 6226 may be an ultra-low particulate air (ULPA) filter.

Referring now to FIGS. 244-245 and FIG. 248, filtered air from the filter 6226 may pass to a controlled environment supply plenum 6228. The controlled environment supply plenum 6228 may be disposed upstream of the processing chamber 4300 and transfer chamber 3506 of the system 10. Air may pass into the processing chamber 4300 through one or more processing chamber diffusion grid 6230.

The height of the controlled environment supply plenum 6228 (distance from output side of filter 6226 to diffusion grid 6230) may be not more than 100-125 mm in certain examples. Flow exiting a filter 6226 typically takes a significant amount of distance for momentum to redistribute and for turbulence to dissipate. ISO cleanroom standards (e.g. ISO-14644-4 (2022)), for example, indicate testing for unidirectional airflow is typically conducted at a distance of 150 mm-300 mm from a filter face. Despite the short distance in certain example embodiments, steady air flow at velocities of at least 0.2 m/s with little turbulence and no stagnant zones may be achieved within the environmental supply plenum 6228 a short distance (e.g. ~40-55 mm) from the filter 6228 due to the inclusion of the upstream turbulent kinetic energy dampeners. The environment supply plenum 6228 may, for example, conform with ISO 5 cleanroom criteria. Thus, the inclusion of upstream turbulent kinetic energy dampeners may assist in creating a highly compact controlled environment where filling of bags 26 may acceptably occur relatively close to the filter 6226. For example, the tip of a dispensing sharp 2604 (see, e.g., FIG. 103C) may be within 300 mm of the filter 6226 (e.g. 250-275 mm).

The processing chamber diffusion grids 6230 may be perforated panels which establish a pressure drop between the supply plenum 6228 and the processing chamber 4300. The size and arrangement of perforations for any diffusion grids described herein may be adjusted to generate the desired pressure drop at the minimum anticipated flowrate of air therethrough during operation. In some examples, the processing chamber diffusion grids 6230 may be arranged to be turbulent kinetic energy dampeners and include an array of channels similar to the laminarizers 6218, 6222. The pressure drop engendered by the processing chamber diffusion grid(s) 6230 may be a minimum of 5 Pa. In some embodiments, the pressure drop may be 7.5-15 Pa or higher (the same may be true for other diffusion grids described herein). In certain examples, the perforations in the processing chamber diffusion grids 6230 may be substantially uniform. Alternatively and as shown, the density of perforations or open area of the processing chamber diffusion grids 6230 may vary by grid or within a particular grid. Areas where more back pressure is expected in the processing chamber 4300 may have more open space or denser perforations in the aligned portion of the upstream diffusion gridding. Some regions of the processing chamber diffusion grids 6230 may optionally be solid or devoid or perforations (see, e.g., immediate vicinity of the clean air outlet 3090).

A pressure sensor 6314 may be in communication with the filtration chamber 6224 (e.g. upstream of the filter 6226) and may output a signal which is monitored by the control system 15. A pressure sensor 6316 and a particulate sensor 6318 may be included in the controlled environment supply plenum 6228 and may be in data communication with the control system 15. The control system 15 may compare the pre filter pressure sensor 6314 output to the post filter pressure sensor 6316 to confirm a predetermined expected pressure drop through the filter 6226 is observed. In the event that the pressure drop is less than a predefined expected value, the control system 15 may generate a fault and operation of the system 10 may be prohibited. In the event that the pressure drop is greater than another predefined expected value, the control system 15 may also generate a fault and operation of the system 10 may be prohibited. The control system 15 may similarly generate a fault and inhibit operation of the system 10 in the event that the data signal from the post filter particulate sensor 6318 is indicative of a particulate count about a certain threshold. A supply plenum humidity sensor 6319 may also be included in the controlled environment supply plenum 6228 as well in some embodiments. The control system 15 may be in data communication with the humidity sensor and may generate a fault in the event that the humidity indicated by the output signal from the supply plenum humidity sensor is outside of a predefine range. Other sensors are also possible. For example, at least one anemometer (e.g. hot wire type anemometer) may be in communication with the environmental supply plenum 6228 and may output data indicative of the velocity of airflow. Fluorescence based viable particle counters in communication with the environment supply plenum 6228 may also be included. The control system 15 may verify that the data output from any sensors conforms with respective limits, predefined targets (or ranges), and allowable rates of change. The control system 15 may also verify that data from sensors correlates in an expected manner. For example, if a viable particle counter indicates a high level of viable counts, this may be checked against counts from a non-viable particulate counter. If an expected correlation is not present (e.g. non-viable particulate counts less than viable particulate counts), a fault may be generated. Similarly, if a variation between two sensors (e.g. anemometers) measuring the like characteristics exceeds a predefined threshold, a fault may be generated.

As mentioned above, the example air handling system 6200 supports the establishment of multiple zones of varying environmental control stringency. In some examples, the processing chamber 4300 may be divided into a plurality of zones. A first zone may be established in the airflow channel 2802 (see, e.g., FIG. 118) of the cartridge 2606. The remainder of the processing chamber 4300 may be established as a second environmental control zone or supporting zone. The first zone may be more stringently controlled than the supporting zone. The first zone may be relatively small (e.g. less than 150 or 125 ml). The remainder of the processing chamber 4300 may have a volume of 50-60 liters.

The different zones may be differentiated based on one or more environmental control characteristics. The non-viable particle concentration in various zones may be controlled to differing levels. For example, the first zone may be controlled to have fewer than some threshold number of particles larger than 0.3 or 0.5 μm (or may satisfy threshold criteria for a number of respective particulate sizes). The supporting zone may permit a greater number of such particles up to a second, larger predetermined threshold (or may have higher allotments allowed for thresholds respective to the same particle sizes). The viable particulate concentration in various zones may be controlled to differing levels. The degree of unidirectionality of airflow in each zone may also differ. For example, the turbulence intensity in the air flow entering the first zone may average less than 2.25% (e.g. 1-2%). The turbulent intensity in the supporting zone may be greater. The speed of air flowing through the various zones may differ. For example, a high velocity may be present in the first zone while a slower velocity (e.g. 0.45 m/s+/−20%) may be maintained in the supporting zone. The pressure within each zone may be controlled to differing set points as well. For example, the more stringent zone (e.g. the first zone) may be at a higher pressure (at least 5 Pa) than an adjacent, less stringently controlled zone. The pressure delta and flow rate out of the more stringently control zone may be sufficient to establish an aerodynamic boundary. The number of air exchanges per unit time may differ between zones. Set points for any environmental control characteristics may be taken from an ISO clean room standard. At least some set points for the first zone may be borrowed from an ISO 5 (or more stringent) standard while at least some set points for the supporting zone may be borrowed from an ISO 7 (or more stringent) standard. The first zone may, for example, be monitored to ensure conformity to particulate standards for an ISO 5 cleanroom and the supporting zone may be monitored to ensure compliance with particle standards for an ISO 7 cleanroom.

Though each zone within the processing chamber 4300 may receive air from the supply plenum 6228 (which may be a third control zone), the shape of the bore 3097 of the clean air output 3090 may engender a different pressure and flowrate therethrough (the air flow may also be conditioned by vanes or other conditions where included in the clean air output 3090). Thus, the airflow channel 2802 of the cartridge 2606 may be maintained at a more stringent level of environmental control despite being in fluid communication with the supporting zone. In certain examples, with the blower 6210 outputting 250 m³ per hour, the first zone may be subjected to 300-400 air exchanges per minute (e.g. 350 air exchanges per minute). The supporting zone may be subjected to 70-85 air exchanges per minute (e.g. 75 per minute). Though the air flow through the supply plenum 6228 may be steady at velocities of at least 0.2 m/s with little turbulence and no stagnant zones, the average velocity range in a core zone of the supply plenum 6228 may be particularly tight. The core zone may be the volume within the supply plenum 6228 adjacent to, contiguous with, and directly upstream the clean air output 3090. Average velocities measured at different locations along a line parallel to the exit face of the filter 6226 extending through the core zone may have an average velocity deviation less than, for example, +/−10% off a predefined velocity target set point over a number of of diversion valve 6240 positions (ranging from fully closed to fully open). Measurements may be taken at a distance of 70-80 mm from the exit face of the filter 6226 for example.

The first zone and supporting zone may be merged into a single zone in certain scenarios. For example, when a cartridge 2606 is replaced, the entire volume of the processing chamber 4300 may be merged into a single zone of environmental control. The entirety of the processing chamber 4300 may be maintained at or substantially at the level of stringency of the first zone. When a cover 2992 (see, e.g., FIG. 114) is removed from a new cartridge 2606, the airflow channel 2802 may be exposed, but be out of contact with the clean air output 3090. To support exposure of the airflow channel 2802 to a space which would otherwise be part of the less stringently controlled supporting zone, the whole processing chamber 4300 may be held at the control stringency of the first zone. The clean air output 3090 may then be actuated into contact with the airflow channel 2802 of the cartridge 2606 (further described in relation to FIG. 116). The first and supporting zones may then diverge to different levels of environmental control.

The processing chamber 4300 may include at least one pressure sensor 6320 and at least one particulate sensor 6322 in data communication with the control system 15. The control system 15 may compare the pressure sensor 6320 output to the pressure data from the post filter pressure sensor 6314 in the controlled environment supply plenum 6228. In the event that the pressure drop indicated by the data from the pressure sensor 6320 and the post filter pressure sensor 6314 is outside of a predefined expected range, the control system 15 may trigger a fault. Further use of the system 10 may be prohibited. In the event that data from the particulate sensor 6322 indicates particulate levels above a predetermined threshold, the control system 15 may similarly generate a fault and prohibit further use of the system 10. Other sensors such as anemometer(s) or fluorescence based viable particle counters may also be in communication with a processing chamber 4300. Any sensors may be monitored by the control system 15 for conformity to predefined criteria or other expected behavior and trends (e.g. as described above in relation to sensing in the supply plenum 6228).

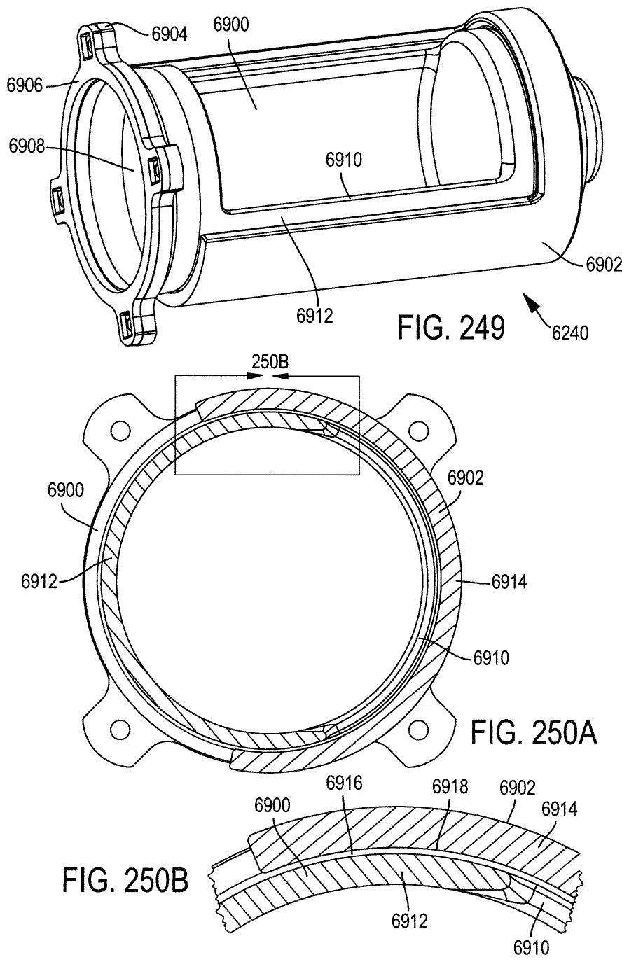

Still referring to FIGS. 244-245 and FIG. 248, air may also pass from the supply plenum 6228 to a transfer chamber plenum 6242 via a diversion valve assembly 6240 (further described in relation to FIGS. 249-250B). The diversion valve assembly 6240 may be actuated to adjust the amount of airflow which passes from the supply plenum 6228 to the transfer chamber plenum 6242. The diversion valve assembly 6240 may also be actuated to a sealing state which isolates the supply plenum 6228 from the transfer chamber plenum 6242. That is, the diversion valve assembly 6240 may be actuated to a state in which it establishes an aerodynamic boundary between its inlet and outlet. In the sealing state, the flowrate of air through the diversion valve assembly 6240 may be very rapid (e.g. 6 m/s) such that the diversion valve assembly 6240 inhibits any fluid communication from the transfer chamber plenum 6242 to the supply plenum 6228.

A pressure sensor 6324 and a particulate sensor 6326 may be provided in the transfer chamber plenum 6242. Each may be in data communication with the control system 15. The control system 15 may monitor the signal output by the particulate sensor 6326. In the event the data signal is indicative that particulate levels in the transfer chamber plenum 6242 have breached a predefined threshold, the control system 15 may generate a fault. Further operation of the system 10 may be inhibited. The diversion valve assembly 6240 may be actuated by the control system 15 based at least in part on the data from the pressure sensor 6324 in the transfer chamber plenum 6242 and other pressure sensors (e.g. 6316, 6320, 6324) in the air handing assembly 6200. The control system 15 may also compare the data signal from the pressure sensor 6324 to the output of a pressure sensor upstream of the diversion valve assembly 6240 (e.g. post ULPA pressure sensor 6316) to verify that an expected pressure drop is observed. The expected pressure drop may be specified a range which is specific to the position of the diversion valve assembly 6240. Other sensors such as anemometer(s) or fluorescence based viable particle counters may also be in communication with a transfer chamber plenum 6242. Any sensors may be monitored by the control system 15 for conformity to predefined criteria or other expected behavior and trends (e.g. as described above in relation to sensing in the supply plenum 6228).

From the transfer chamber plenum 6242, air may flow into the transfer chamber 3506 through at least one transfer chamber diffusion grid 6244. The transfer chamber diffusion grid 6244 may be a perforated panel which establishes a pressure drop between the transfer chamber plenum 6242 and the transfer chamber 3506 (at least when the environment in the transfer chamber 3506 is controlled to certain set points). In some examples, the transfer chamber diffusion grid 6244 may be arranged to be a turbulent kinetic energy dampener and include an array of channels similar to the laminarizers 6218, 6222. The transfer chamber 3506 may, in certain examples, have a volume of 15-20 liters.

A pressure sensor 6330 and a particulate sensor 6332 may be in communication with the transfer chamber 3506. Each may be in data communication with the control system 15. The control system 15 may monitor the pressure delta between the transfer chamber plenum 6242 and the transfer chamber 3506. In the event that the pressure delta is outside of a predefined range, the control system 15 may generate a fault. The control system 15 may also inhibit further operation of the system 10. The output from the particulate sensor 6332 may also be monitored and compared against a predefined threshold by the control system 15. In the event that the data signal from the particulate sensor 6332 indicates particulate levels are in breach of the predefined threshold, the control system 15 may generate a fault. Further operation of the system 10 may be prohibited. Other sensors such as anemometer(s) or fluorescence based viable particle counters may also be in communication with a transfer chamber 3506. Any sensors may be monitored by the control system 15 for conformity to predefined criteria or other expected behavior and trends (e.g. as described above in relation to sensing in the supply plenum 6228).

When the door 4702 between the transfer chamber 3506 and the outfeed chamber 4202 is opened, the environment within the transfer chamber 3506 may be uncontrolled. The diversion valve assembly 6240 may be adjusted to establish and maintain a control zone within the transfer chamber 3506 when the transfer chamber 3506 is isolated from the outfeed chamber 4202. In some examples, the transfer chamber 3506 may be brought to a level of environmental control substantially equivalent to the supporting zone in the processing chamber 4300. The transfer chamber 3506 may be held at this level of environmental control for a period of time before the control system 15 permits the door 4700 between the transfer chamber 3506 and the processing chamber 4300 to be opened. This period of time may be referred to herein as a recovery period. The recovery period may ensure that the transfer chamber 3506 is flushed with air and at a desired level of environmental control after exposure to the outfeed chamber 4202. If data from the particulate sensor 6332 (or another environmental control sensor in the transfer chamber 3506) breaches a predefined threshold during the recovery period a timer for the recovery period may be reset (the number or retries or resets may be capped). In certain embodiments, the transfer chamber 3506 may be subjected to 55-65 air exchanges a minute when the diversion valve assembly 6240 is in a fully open state (which may be the state typically used during the recovery period).

The door actuator for door 4700 may be commanded to open the door 4700 relatively slowly. This may help to ensure minimal disruption of airflow unidirectionality. In some embodiments, the door 4700 may be opened at a rate of less than 15° per second (e.g. 10-8°/sec or less).

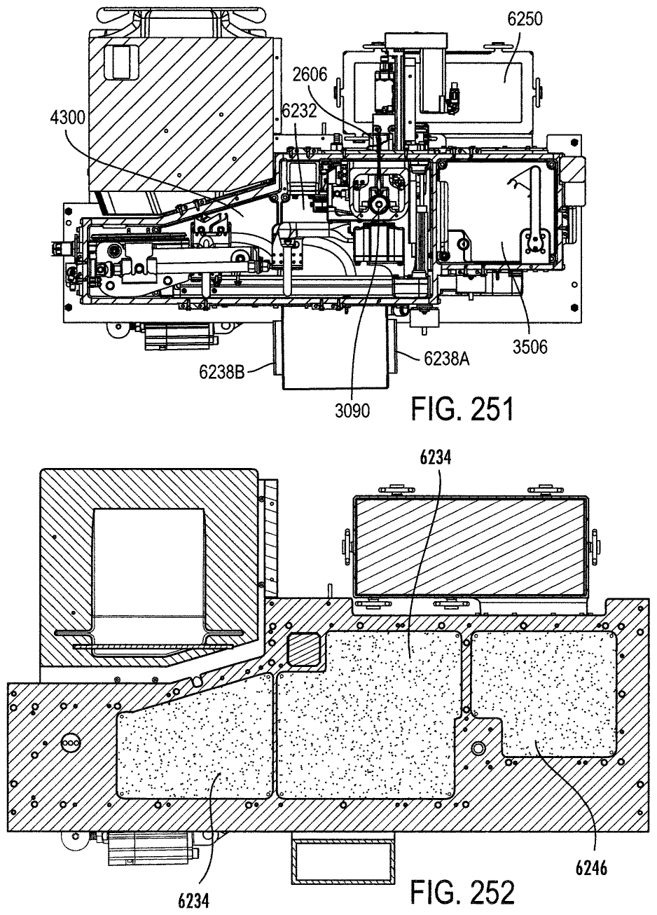

Referring now to FIGS. 244-245 and FIGS. 251-252, air may pass through the processing chamber 4300 and transfer chamber 3506 and exit the chambers 4300, 3506 via respective exhaust gridding 6234, 6246. The exhaust gridding 6234, 6246 for each chamber or compartment may be perforated panels which establishes a pressure drop between the compartments and downstream exhaust chambers 6236, 6248.

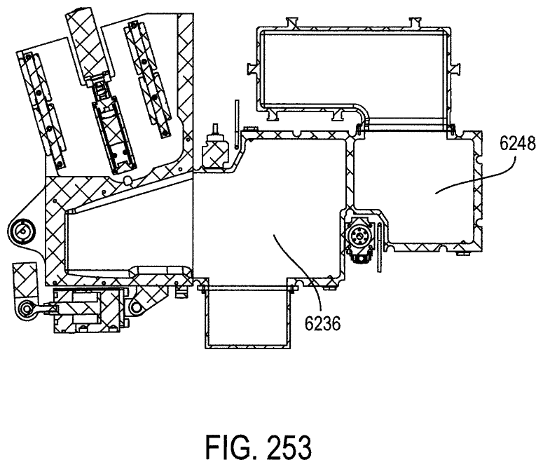

Referring now to FIGS. 244-245 and FIGS. 253, the exhaust chamber 6236 for the processing chamber 4300 may be partitioned from the exhaust chamber 6248 for the transfer chamber 3506. This may ensure that no communication between the two exhaust chambers 6236, 6248 is possible. Thus, a high pressure in one exhaust chamber 6236, 6248 would not cause air to backflow through the exhaust gridding 6234, 6246 in the other of the exhaust chambers 6236, 6248.

A pressure sensor 6328, 6334 may be in communication with each of the exhaust chambers 6236, 6248. The control system 15 may monitor an output signal from each pressure sensor 6328, 6334. These signals may be compared respectively to the output of the pressure sensors 6320, 6330 in the processing compartment 4300 and the transfer chamber 3506. The control system 15 may verify that a predefined expected pressure drop is present through the respective exhaust gridding 6234, 6246 associated with each exhaust chamber 6236, 6248. In the event that the pressure drop is less than the predefined expected amount, the control system 15 may generate a fault. The control system 15 may prohibit further operation of the system 10.

Each of the exhaust chambers 6236, 6248 may include at least one leak sensor 6338, 6340 in data communication with the control system 15. The leak sensors 6338, 6340 may, for example, be pairs of electrodes in certain examples. The leak sensors 6338, 6340 may be placed in a low point in the exhaust chambers 6236, 6248. In the event that liquid is present, the control system 15 may generate a fault and may require a service event before permitting further operation of the system 10. The outfeed chamber 4202 may include a catch tray, plate, or other guard positioned at the bottom of the compartment. The outfeed chambers 4202 may similarly include one or more leak sensor.

Air may flow through the processing compartment exhaust chamber 6236 to at least exhaust filter 6238A, B (two are shown). Air may flow through the transfer exhaust chamber 6248 to at least one exhaust filter 6250. Air exiting the exhaust filters 6238A, B, 6250 may pass into the outfeed compartment 4202 or another portion of the enclosure 12. This air may also be recirculated within the enclosure 12 and drawn back into the pre-filter 6204.

Referring now to FIGS. 249-250B, various views of an example diversion valve assembly 6240 are shown. The example diversion valve assembly 6240 may include a stator 6900 and a rotor 6902. The stator 6900 may be coupled to a wall of the transfer chamber plenum 6242 (see, FIG. 248). Components of the diversion valve assembly 6240 may be constructed of a metallic material (e.g. aluminum). In the example, the stator 6900 includes an elongate body 6912 with a round cross-sectional shape. As shown, a flange 6904 is included at an end of the stator 6900 with a variety of coupling points for attaching the stator 6900 to the wall of the transfer chamber plenum 6242. A gasket 6906 may also be included to form a fluid tight coupling. Air may flow from the supply plenum 6228 through a passage in the wall of the transfer chamber plenum 6242 and through an open end of the stator 6900 and into a stator bore 6908. The end of the stator 6900 opposite the open end may closed.

As shown, the stator 6900 includes an outlet 6910. In the example embodiment, the outlet 6910 is in the form of a fenestration extending through the sidewall of the elongate body 6912. The fenestration has a length dimension at least 80% of the axial dimension of the elongate body 6912. The width of the fenestration is constant though could vary in alternative embodiments. The fenestration may be present over an arc of 100-170° (e.g. 123° or 150°) in the sidewall of the elongate body 6912. As shown, all corner surfaces of the fenestration may be rounded to facilitate ease of cleaning.

As shown, the rotor 6902 includes a cradle portion 6914 which partially surrounds and envelops the portion of the stator 6900 including the outlet 6910. The cradle portion 6914 may extend from an end plate of the rotor 6902 which is coupled to the output of a rotary actuator 6243. The cradle portion 6914 may be formed as a piece of material extending along an arc greater than that of the fenestration in the sidewall of the elongate body 6912. The arc of the cradle portion 6914 may be 10-120° (e.g. 85°) greater than that of the fenestration. This may allow the outlet 6910 of the stator 6900 to be both completely covered by the cradle portion 6914 and completely unobstructed by the cradle portion 6194 depending on the rotation position of the rotor 6902. The control system 15 may issue commands to orchestrate actuation of the rotor 6902 such that a desired percentage of the outlet 6910 is covered by the cradle portion 6914. The control signal from a rotary position sensor (e.g. an encoder in the rotary actuator 6243) may be monitored to inform the commands issued to the rotary actuator 6243 by the control system 15. The air flow and pressure downstream of the diversion valve 6240 may be adjusted by altering the amount of the outlet 6910 covered by the cradle portion 6914. Pressure and airflow into the processing chamber 4300 may also be modified by adjusting the position of the rotor 6902. The target percentage of the outlet covered by the cradle portion 6914 may be determined based on data from pressure and air flow sensors monitoring the relevant characteristics downstream of the diversion valve 6240 and within the processing chamber 4300. In certain examples, when the blower is outputting air at a flowrate of 250 m³/hr, the diversion valve assembly 6240 may output a flowrate of 65-75 m³/hr (e.g. 70 m³/hr) when fully open.

As mentioned elsewhere herein, the diversion valve 6240 may also be driven to a state in which an aerodynamic boundary is formed between the upstream and downstream sides of the diversion valve 6240. This may ensure communication from the transfer chamber plenum 6242 and into the supply plenum 6228 is prevented. As best illustrated in FIG. 250B, the radius of the arced cradle portion may be slightly greater (e.g. 2.5-7.5%) than the radius for the sidewall of the elongate body 6912. When the diversion valve 6240 is fully closed, the cradle portion 6914 may completely obstruct the outlet 6910 of the stator 6900. Lateral sections of the cradle portion 6914 may envelop a solid portion of the elongate body 6912 flanking each side of the outlet 6910. There may, however, be a small gap between the exterior surface 6916 of the elongate body 6912 and the inner surface 6918 of the cradle portion 6914. This provides a flow path through the diversion valve 6240, albeit one with a small total cross-sectional area. As a result, the velocity of flow out of the diversion valve 6240 through this gap will be considerably higher than air velocities encountered when the outlet 6910 is unobstructed or partially obstructed. Preferably, the gap may engender velocities of 6 m/s or higher at a flow rate of 15 m³/hr. This may effectively create a one way seal isolating the supply plenum 6228 from the transfer chamber plenum 6242.

The stator 6900 may differ in some examples. In alternative embodiments, a stator 6900 may not include an outlet 6910 formed by a single fenestration. Instead, the stator 6900 may include a number of perforations which form the outlet 6910. Perforations may be present over a 100-170° arc of the sidewall of the elongate body 6912. In certain examples, the perforations may be uniform in size and/or spacing. In other embodiments, the density of perforations may increase or decrease as distance toward the closed end of the elongate body 6912 increases. The diameter of the perforations may be constant or may increase as proximity to the closed end of the elongate body 6912 increases. When the diversion valve 6240 is in a fully open state all of the perforations may be unobstructed. The total open area of the outlet 6910 afforded by the perforations may be decreased by rotating the cradle portion 6914 of the rotor 6902 to obstruct or partially obstruct some of the perforations. The rotor 6902 may be displaced via commands from the control system 15 to the rotary actuator 6243 until a target percent open area of the outlet 6910 is achieved.

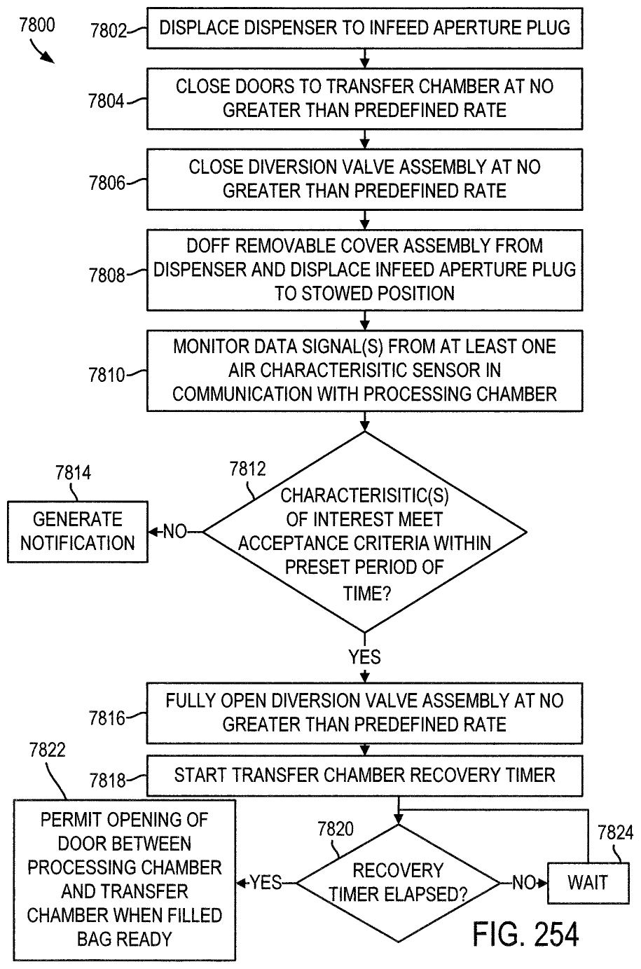

Referring now to FIG. 254, a flowchart 7800 detailing a number of example actions which may be executed to place the interior of a dispenser 5000 into communication with a processing chamber 4300 is depicted. As shown, in block 7802, the dispenser 5000 may be displaced to the infeed aperture plug 5502 (see, e.g., FIGS. 46A-B and FIG. 63). The doors 4700 and 4702 of the transfer chamber 3506 (see, e.g., FIG. 144A) may be displaced to a closed position in block 7804. The doors 4700, 4702 may be displaced at no greater than a predefined rate. In some examples, the rate may be between 6-10° per second (e.g. 7.2° per second). The predefined rate may be selected to ensure any turbulence generated as a door 4700, 4702 displaces is kept below a threshold. The predefined rate may also be selected to ensure that depressurization/pressurization of chambers occurs at less than a threshold rate.

In block 7806, a diversion valve assembly 6240 controlling communication between the controlled environment supply plenum 6228 and transfer chamber plenum 6242 (see, e.g., FIG. 248) may be actuated to a fully closed position. The diversion valve assembly 6240 may be actuated to the closed position at no greater than a predefined rate. In some embodiments, the predefined rate may be between 7.5-15° per second (e.g. 10° per second). The predefined rate may be selected to limit introduction of turbulence below a predefined threshold and limit particulate generation as the diversion valve assembly 6240 is displaced. The predefined rate may also assist in lowering the rate of pressure changes engendered with actuation of the diversion valve assembly 6240.

In block 7808, the removable cover assembly 5002 of the dispenser 5000 may be doffed (see, e.g., FIGS. 46A-B and FIG. 64) and the infeed aperture plug 5502 may be displaced to a stowed position (see, e.g., FIG. 2). With the diversion valve assembly 6240 and door 4700 to the transfer chamber 3506 closed, backflow from the transfer chamber 3506 and transfer chamber supply plenum 6242 may be prevented as the interior volume of the dispenser 5000 is brought into communication with the processing chamber 4300. The control system 15 may monitor, in block 7810, data signal(s) from at least one air characteristic sensor (e.g. pressure sensor(s), particulate sensor(s), flow rate sensor(s), etc.) in communication with the processing chamber 4300. The data signal(s) may be analyzed to ensure that one or more predefined acceptance criteria are met. For example, pressure may be required to conform to one or more pressure stability threshold and may be required to be above a predefined value. It may also be required that a drop in pressure of at least a certain magnitude followed by a repressurization to at least a certain value is observed. Particulate counts may be required to be below preset values. Flow rates may be required to be above an expected value or within a preset range.

If, in block 7812, the data signal(s) are indicative the characteristics of interest fail to meet predefined acceptance criteria within a preset time, a notification may be generated in block 7814. The notification may, for example, be an alarm displayed on a user interface 6100 for the system 10. The control system 15 may also inhibit filling of bags 26 by the system 10. If, in block 7812, the data signal(s) or indicative the characteristics of interest conform to the acceptance criteria, the control system 15 may begin orchestrating processing of bags 26 in the dispenser 5000 by the system 10. The diversion valve assembly 6240 may be actuated to a fully open position in block 7816. The diversion valve assembly 6240 may be displaced at no greater than a predefined rate. In some embodiments, the predefined rate may be between 7.5-15° per second (e.g. 10° per second). The predefined rate may be selected to limit introduction of turbulence below a predefined threshold and limit particulate generation as the diversion valve assembly 6240 is displaced. The predefined rate may also assist in lowering the rate of pressure changes engendered with actuation of the diversion valve assembly 6240.

In block 7818, a recovery timer for the transfer chamber 3506 may be started. If, in block 7820, the recovery timer has elapsed, the control system 7822 may permit opening of the door 4700 between the processing chamber 4300 and the transfer chamber 3506. Filled bags 26 may be displaced from the processing chamber 4300 to the transfer chamber 3506 as described in relation to FIGS. 145-149. In the event that the recovery timer has not elapsed, the control system 15 may wait in block 7824 until the timer has elapsed. Any filled bag 26 may be temporarily held in the processing chamber 4300 until the timer has elapsed. The recovery timer may be selected to ensure at least a predefined number of air exchanges has occurred. Preferably, the control system 15 may also monitor pressure and particulate sensors 6324, 6326 (and/or data from any other relevant environmental control sensors) and the door 4700 may only be opened in the event data from these sensors indicates conformity with respective predefined thresholds, set points, ranges etc. The predefined number of air exchanges may at least be between 2-3. In certain embodiments, the recovery period may be at least 2 seconds. In certain systems 10, this may result in a number of air exchanges being at least 30-60 (e.g. in excess of 60).

The flowchart 7800 shown and described in relation to FIG. 254 as well as those flowcharts 7830, 7850, 7870, and 7890 described in the forthcoming description detail door 4700, 4702, and diversion valve assembly 6240 positions. It shall be understood that doors 4700, 4702, and the diversion valve assembly 6240 may only be displaced if they are not already in the prescribed position. Displacement of the doors 4700, 4702, and the diversion valve assembly 6240 is thus optional and may depend on the current state of these components

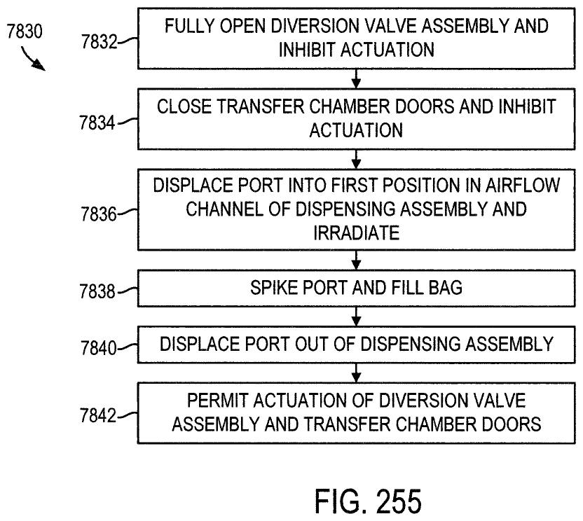

Referring now to FIG. 255, a flowchart 7830 detailing a number of example actions which may be executed to fill a bag 26 with a dispensing assembly 2602 is depicted. As shown, in block 7832 the diversion valve assembly 6240 (see, e.g., FIG. 249) may be actuated to a fully open position. The diversion valve assembly 6240 may be displaced at no greater than a predefined rate. In some embodiments, the predefined rate may be between 7.5-15° per second (e.g. 10° per second). The predefined rate may be selected to limit introduction of turbulence below a predefined threshold and limit particulate generation as the diversion valve assembly 6240 is displaced. The control system 15 may also inhibit further actuation of the diversion valve assembly 6240 once the fully open position is reached. Opening of the diversion valve assembly 6240 may ensure that air may flow out of the controlled environment supply plenum 6228 (see, e.g., FIG. 248) through all regions of the air handling system 6200 having a communication pathway to the controlled environment supply plenum 6228 during filling.

The door 4700 between the processing chamber 4300 and the transfer chamber 3506 may be closed (see, e.g., FIG. 144A) in block 7834. The door 4700 may be displaced at no greater than a predefined rate. In some examples, the rate may be between 6-10° per second (e.g. 7.2° per second). The predefined rate may be selected to ensure any turbulence generated as the door 4700 displaces is kept below a threshold. The predefined rate may also be selected to ensure that depressurization/pressurization of chambers occurs at less than a threshold rate. The control system 15 may also inhibit further actuation of the door 4700 in block 7834.

In block 7836, the port 392 of a bag 26 may be displaced into a first position within the dispensing assembly 6206. The port 392 may also be irradiated in block 7836 by an irradiation assembly 2608 (see, e.g., FIG. 134, FIG. 142B). In block 7838, the port 392 may be spiked and the bag 26 may filled with a desired volume of fluid via a dispensing sharp 2604 of the dispensing assembly 2602 (see, e.g., FIG. 101B, FIG. 142B). The port 392 may be displaced out of the dispensing assembly 2602 in block 7840. Actuation of the diversion valve assembly 6240 and door 4700 may again be permitted by the control system 15 in block 7842. Though described in relation to a port 392 of a bag 26, the air handling system 6200 may be controlled in the same manner when a prime port 3120 (see, e.g., FIG. 129) is brought to the dispensing assembly 2602 during priming of a cartridge 2606 or during a disinfection cycle for a cartridge 2606.

While the above description of FIG. 255 refers to the diversion valve assembly 6240 and door 7400 being maintained in prescribed states during filling of a bag 26, this may not be true in all examples. For example, in some embodiments, a reduced displacement rate may be permitted for each of the diversion valve assembly 6240 and door 7400 while blocks 7836, 7838, and 7842 are executed. The reduced rates may be less than the predefined rates mentioned in relation to blocks 7832, 7834. In still other examples, displacement of the diversion valve assembly 6240 and door 7400 may be permitted (or may be permitted at an increased rate) once the port 392 of a bag 26 being filled has been spiked.

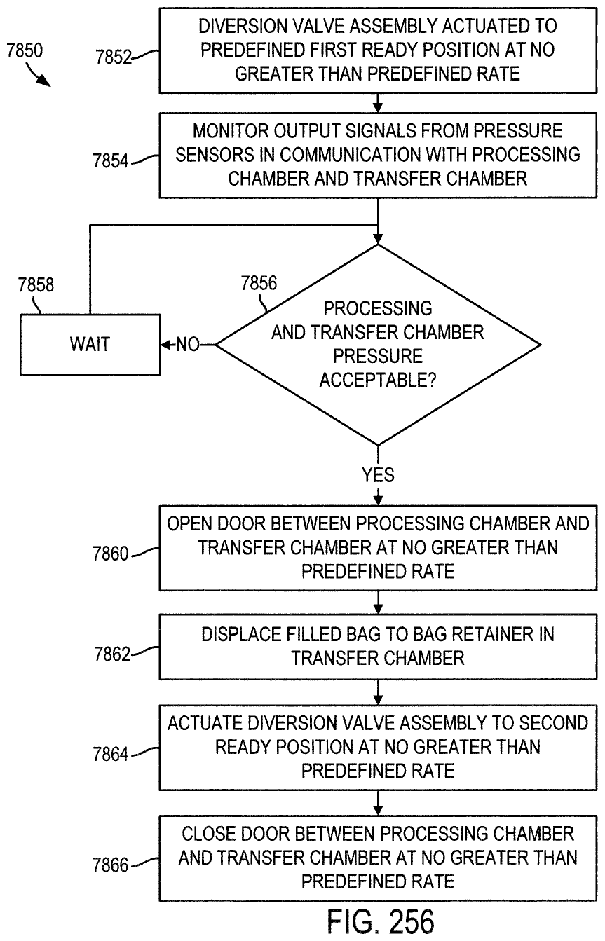

Referring now to FIG. 256, a flowchart 7850 detailing a number of example actions which may be executed to transfer a filled bag 26 into a transfer chamber 3506 is depicted. As shown, in block 7852 the diversion valve assembly 6240 (see, e.g., FIG. 249) may be actuated to a predefined first ready position. The diversion valve assembly 6240 may be displaced at no greater than a predefined rate. In some embodiments, the predefined rate may be between 7.5-15° per second (e.g. 10° per second). The predefined rate may be selected to limit introduction of turbulence below a predefined threshold and limit particulate generation as the diversion valve assembly 6240 is displaced. The first ready position may be a majority closed state (e.g. ⅔ closed). The first ready position may be selected to be a position which causes the transfer chamber 3506 to be substantially equal in pressure to the processing chamber 4300. Alternatively, the first ready position may be selected to maintain the processing chamber 4300 at a slightly higher pressure (2-7.5 Pascal) than that of the transfer chamber 3056. This may assist in limiting generation of turbulence below predetermined thresholds as the door 4700 is opened. It may also assist in keeping a rate of pressure change in each chamber 3506, 4300 below a prescribed threshold as the door 4700 is opened. It may further ensure that displacement of air between chambers 3506, 4300 is from the processing chamber 4300 toward the transfer chamber 3506.

In block 7854, output signals from pressure sensors respectively in communication with the processing chamber 4300 and transfer chamber 3506 may be monitored by the control system 15. If, in block 7856, pressure data from the pressure sensors indicates nonconformity with pressure criteria for the processing chamber 4300 and transfer chamber 3506, the control system 15 may wait up to a preset period of time in block 7858. Preferably, the output signal from one or more particulate sensors (or any other environmental control sensors) in one or both the transfer chamber 3506 and processing chamber 4300 may also be monitored for conformance with predefined criteria. A notification may be generated if the pressure data (and/or particulate, or other environmental control data) fails to indicate conformity is has been achieved within a predefined timeout period. The control system 15 may inhibit operation of the system 10 until an indication service personnel have interacted with the system 10 is received. If, in block 7856, data from the pressure sensors indicates pressure criteria have been met, the door 4700 between the processing chamber 4300 and transfer chamber 3506 may be commanded to open by the control system 15 in block 7860. The door 4700 may be displaced at no greater than a predefined rate. In some examples, the rate may be between 6-10° per second (e.g. 7.2° per second). The predefined rate may be selected to ensure any turbulence generated as the door 4700 displaces is kept below a threshold. The predefined rate may also be selected to ensure that any pressure changes in the chambers 4300, 3506 occurs at less than a threshold rate.

The filled bag 26 may be displaced to a bag retainer 4704 in the transfer chamber 3506 in block 7862. This may be accomplished as described above in relation to FIGS. 145-149. In block 7864, the diversion valve assembly 6240 may be actuated to a second ready position. The diversion valve assembly 6240 may be displaced at no greater than a predefined rate. In some embodiments, the predefined rate may be between 7.5-15° per second (e.g. 10° per second). The predefined rate may be selected to limit introduction of turbulence below a predefined threshold and limit particulate generation as the diversion valve assembly 6240 is displaced. The second ready position may be a majority open position and in some embodiments may be defined to be the same as the first ready position. The door 4700 may be actuated closed in block 7866. The door 4700 may be displaced at no greater than a predefined rate as described above in relation to block 7860.

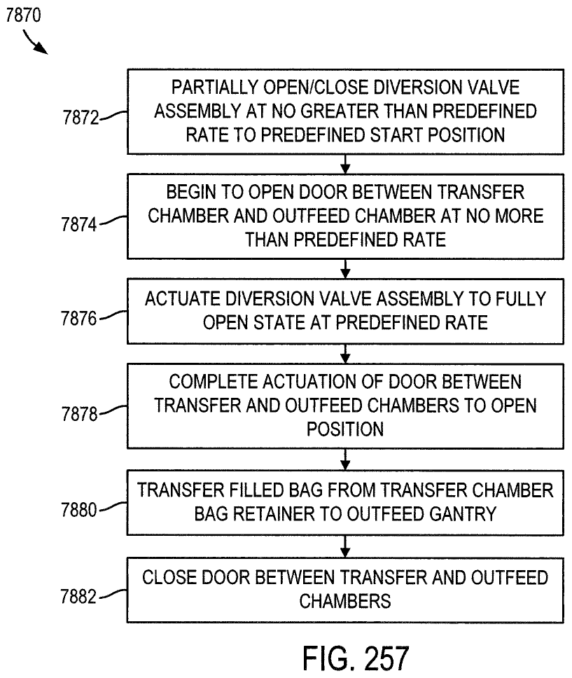

Referring now to FIG. 257, a flowchart 7870 detailing a number of example actions which may be executed to transfer a bag 26 out of a transfer chamber 3506 to an outfeed compartment 4202 (see, e.g., FIG. 221) is depicted. In block 7872, the diversion valve assembly 6240 may be displaced to an intermediary position. The diversion valve assembly 6240 may be displaced at no greater than a predefined rate. In some embodiments, the predefined rate may be between 7.5-15° per second (e.g. 10° per second). The predefined rate may be selected to limit introduction of turbulence below a predefined threshold and limit particulate generation as the diversion valve assembly 6240 is displaced. The intermediary position may be a majority closed state (e.g. at least 75% closed). The door 4702 between the transfer chamber 3506 and the outfeed compartment 4202 (see, e.g., FIG. 150) may be commanded open by the control system 15 in block 7874. The door 4702 may be displaced at no greater than a predefined rate. In some examples, the rate may be between 6-10° per second (e.g. 7.2° per second). The predefined rate may be selected to ensure any turbulence generated as the door 4702 displaces is kept below a threshold. The predefined rate may also be selected to ensure that depressurization of the transfer chamber 3506 occurs at less than a threshold rate. As the door 4702 is displacing to its open state, the diversion valve assembly 6240 may be actuated to a fully open position in block 7876. The diversion valve assembly 6240 may be displaced at no greater than a predefined rate which may be between 15-30° per second (e.g. 20° per second). The predefined rate may be selected to limit introduction of turbulence below a predefined threshold and ensure that the diversion valve assembly 6240 reaches the fully open position at least some amount of time prior to the door 4702 reaching the open state. This may ensure that air is flowing from the transfer chamber 3506 to the outfeed compartment at at least a prescribed velocity (e.g. at least 2 m/s) before the door 4702 reaches the open state in block 7878. This may inhibit ingress of particulate, detritus, pests, etc. into the transfer chamber 3506 while the door 4702 is opened. The filled bag 26 in the transfer chamber 3506 may be collected by a gantry assembly 4200 (or other automation hardware) and displaced into the outfeed compartment in block 7880. This may be accomplished as described in relation to FIGS. 149-151 for example. With the bag 26 removed, the door 4702 may be displaced to a closed position in block 7882. The door 4700 may be displaced at no greater than a predefined rate as described above in relation to block 7874. A recovery timer may be started and may be required to elapse (see, e.g., blocks 7818-7824 of FIG. 254) prior to the door 4700 being permitted to open (other recovery criteria described above may also be imposed).

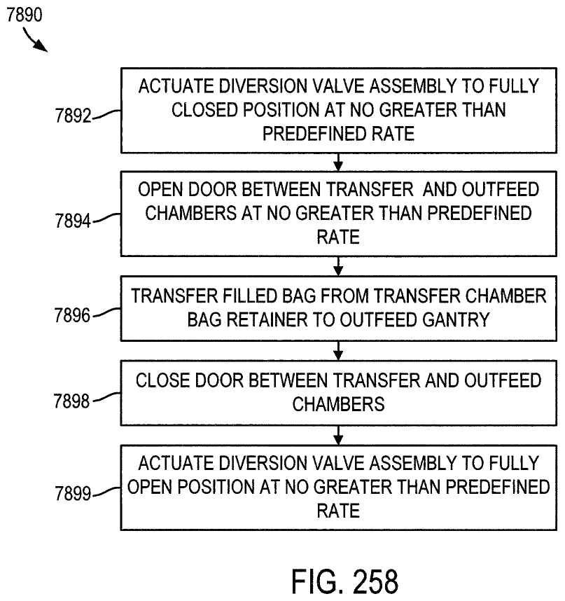

Referring now to FIG. 258, another flowchart 7890 detailing a number of example actions which may be executed to transfer a bag 26 out of a transfer chamber 3506 to an outfeed compartment 4202 (see, e.g., FIG. 221) is depicted. As shown, in block 7892, the diversion valve assembly 6240 may be actuated to a fully closed position. The diversion valve assembly 6240 may be displaced at no greater than a predefined rate. In some embodiments, the predefined rate may be between 7.5-15° per second (e.g. 10° per second). The predefined rate may be selected to limit introduction of turbulence below a predefined threshold and limit particulate generation as the diversion valve assembly 6240 is displaced. In block 7894, the door between the transfer chamber 3506 and the outfeed compartment 4202 may be opened. The door 4702 may be displaced at no greater than a predefined rate. In some examples, the rate may be between 6-10° per second (e.g. 7.2° per second). The predefined rate may be selected to ensure any turbulence generated as the door 4702 displaces is kept below a threshold. The predefined rate may also be selected to ensure that depressurization of the transfer chamber 3506 occurs at less than a threshold rate. Where predefined rates are discussed in relation to FIGS. 254-258, it shall be understood, displacements may also be controlled such that the rate of change in displacement is also maintained within predetermined limits. In alternative examples, the door 4702 may be opened rapidly as the diversion valve assembly 6240 is in a closed state.

As the diversion valve assembly 6240 is in a state in which the processing chamber 4300 is isolated from the transfer chamber, the diversion valve assembly 6240 may inhibit ingress of particulate, detritus, pests, etc. into the processing chamber 4300 while the door 4702 is open. The filled bag 26 in the transfer chamber 3506 may be collected by a gantry assembly 4200 (or other automation hardware) and displaced into the outfeed compartment in block 7896. This may be accomplished as described in relation to FIGS. 149-151 for example. With the bag 26 removed, the door 4702 may be displaced to a closed position in block 7898. The door 4700 may be displaced at no greater than a predefined rate as described above in relation to block 7874. Alternatively, the door may be displaced rapidly. The diversion valve assembly 6240 may be actuated to a fully open position in block 7899. The diversion valve assembly 6240 may be displaced at no greater than a predefined rate. In some embodiments, the predefined rate may be between 7.5-15° per second (e.g. 10° per second). The predefined rate may be selected to limit introduction of turbulence below a predefined threshold and limit particulate generation as the diversion valve assembly 6240 is displaced. A recovery timer may be started and may be required to elapse (see, e.g., blocks 7818-7824 of FIG. 254) prior to the door 4700 being permitted to open (other recovery criteria described above may also be imposed).

Various alternatives and modifications can be devised by those skilled in the art without departing from the disclosure. Accordingly, the present disclosure is intended to embrace all such alternatives, modifications, and variances. Additionally, while several embodiments of the present disclosure have been shown in the drawings and/or discussed herein, it is not intended that the disclosure be limited thereto, as it is intended that the disclosure be as broad in scope as the art will allow and that the specification be read likewise. Therefore, the above description should not be construed as limiting, but merely as exemplifications of particular embodiments. And, those skilled in the art will envision other modifications within the scope and spirit of the claims appended hereto. Other elements, steps, methods, and techniques that are insubstantially different from those described above and/or in the appended claims are also intended to be within the scope of the disclosure.

The embodiments shown in drawings are presented only to demonstrate certain examples of the disclosure. And, the drawings described are only illustrative and are non-limiting. In the drawings, for illustrative purposes, the size of some of the elements may be exaggerated and not drawn to a particular scale. Additionally, elements shown within the drawings that have the same numbers may be identical elements or may be similar elements, depending on the context.

Where the term “comprising” is used in the present description and claims, it does not exclude other elements or steps. Where an indefinite or definite article is used when referring to a singular noun, e.g. “a” “an” or “the”, this includes a plural of that noun unless something otherwise is specifically stated. Hence, the term “comprising” should not be interpreted as being restricted to the items listed thereafter; it does not exclude other elements or steps, and so the scope of the expression “a device comprising items A and B” should not be limited to devices consisting only of components A and B.

Furthermore, the terms “first”, “second”, “third” and the like, whether used in the description or in the claims, are provided for distinguishing between similar elements and not necessarily for describing a sequential or chronological order. It is to be understood that the terms so used are interchangeable under appropriate circumstances (unless clearly disclosed otherwise) and that the embodiments of the disclosure described herein are capable of operation in other sequences and/or arrangements than are described or illustrated herein.

1. A system for packaging fluid into a medical reservoir comprising:
a medical fluid production assembly;
a dispenser containing a chain of reservoirs;
an enclosure having a plurality of compartments and an infeed aperture;
an air handling assembly including an intake, a blower, at least one filter, and an output in communication with the enclosure;
a dispenser interface assembly including a dispenser outlet cover retainer assembly displaceable between a stowed position and an infeed aperture plugging position;
a reservoir singulation assembly;
a filling assembly including a cartridge having an inlet in fluid communication with the medical fluid production assembly and outlet including a dispensing sharp;
a particulate inspection assembly;
a marking assembly;
an outfeed assembly including a plurality of reservoir receptacles;
a database arrangement; and
a controller in data communication with and receiving process data from the medical fluid production assembly, air handling assembly, dispenser interface assembly, reservoir interface assembly, filling assembly, particulate inspection system, marking assembly, and configured to collect and analyze the process data into analyzed data, serialize at least a portion of the process data and at least a portion of the analyzed data into at least one reservoir processing record and communicate each of the at least one reservoir processing record to the database arrangement.

2-399. (canceled)

400. A method of packing fluid into a medical reservoir comprising:
loading a dispenser against an enclosure;
removing and stowing an outlet cover of the dispenser within the a first compartment of the enclosure;
drawing a chain of reservoirs into the first compartment from the dispenser;
singulating an individual reservoir from the chain;
filling the individual reservoir with fluid from a medical fluid production assembly into the individual reservoir through a dispensing sharp which has pierced a septum of the individual reservoir;
displacing and isolating the individual reservoir into a second compartment of the enclosure;
displacing the individual reservoir into a third compartment of the enclosure;
inspecting the individual reservoir;
marking the individual reservoir with a marking dependent on the outcome of the inspection; and
displacing the individual reservoir to a receptacle, the receptacle depending on the outcome of the inspection.

401. The method of claim 400, wherein the method further comprises environmentally controlling a plurality of zones within the enclosure, the zones being in at least one compartment of an enclosure, a first of the plurality of zones directly adjacent the dispensing sharp and a second of the plurality of zones comprising a remainder of the first compartment, the first of the plurality of zones more stringently controlled than the second.

402. The method of claim 401, wherein the second compartment is one of the plurality of zones and the second compartment is controlled to a level of control at least equal to a level of control of the second of the plurality of zones before the individual reservoir is displaced into the second compartment.

403. The method of claim 400, wherein removing and stowing the outlet cover comprises forming a seal between the outlet cover and exposed portions of an interface assembly and retaining the outlet cover on the interface assembly.

404. The method of claim 400, wherein drawing the chain into the first compartment comprises grasping a lead reservoir of the chain and dislodging it from a retainer on a reservoir presenter of the dispenser.

405. The method of claim 400, wherein singulating the individual reservoir from the chain comprises driving a separator assembly of a bag individualizing assembly through a weakened region of the chain.

406. The method of claim 400, wherein filling the individual reservoir comprises irradiating at least an exposed portion of a septum of the individual reservoir and irradiating a portion of the dispensing sharp.

407. The method of claim 400, wherein inspecting the individual reservoir comprises agitating the individual reservoir and agitating the individual reservoir comprises rotating the individual reservoir between pairs of rotational orientations in accordance with a predefined motion profile.

408. The method of claim 400, wherein inspecting the individual reservoir comprises providing a contrasting pattern on a first side of the individual reservoir and illuminating the individual reservoir with collimated, polarized light from an external illuminator.

409. The method of claim 408, wherein inspecting the reservoir further comprises capturing a plurality of images with an imager associated with a polarizing filter, processing the images, detecting pixel clusters of interest within the images and determining classifications for the pixel clusters of interest.

410. The method of claim 409, wherein the marking indicates the individual reservoir is not to be used when at least one pixel cluster of interest is assigned a class predetermined to be unacceptable.

411. The method of claim 409, wherein when at least one pixel cluster of interest is assigned a class predetermined to be unacceptable, the receptacle is an access controlled receptacle.

412. The method of claim 400, wherein marking the individual reservoir comprises embossing the marking into a portion of the individual reservoir and the receptacle is included in an outfeed drawer.

413. The method of claim 400, wherein the method further comprises verifying the marking matches an intended marking, the receptacle dependent upon the inspection and the verification.

414. A system for packaging fluid into a medical reservoir comprising:
a medical fluid production assembly;
a dispenser containing a chain of reservoirs;
an enclosure having a plurality of compartments, a first of the compartments including an infeed aperture
a dispenser interface assembly including a dispenser outlet cover retainer assembly displaceable between a stowed position and an infeed aperture plugging position;
a reservoir singulation assembly in the first compartment;
a filling assembly including cartridge having an inlet in fluid communication with the medical fluid production assembly and outlet including a dispensing sharp, the cartridge at least partially in the first compartment;
a second compartment, there being a first door between the first and second compartments;
a third compartment, there being a second door between the second and third compartments;
a particulate inspection system in the third compartment;
a marking assembly in the third compartment; and
an outfeed assembly in the third compartment including at least one a first array of reservoir receptacles and at least one a second array of reservoir receptacles.

415. The system of claim 414, wherein the medical fluid production assembly includes a plurality of water treatment apparatuses selected from a group consisting of a carbon filter, a reverse osmosis filter, an electrodeionization assembly, and a UV light emitter.

416. The system of claim 414, wherein the chain of reservoirs is a chain of abreastly chained IV bags.

417. The system of claim 414, wherein each reservoir in the chain of reservoirs includes at least one concentrate selected from a list consisting of a saline brine, a sodium chloride brine, a sugar solution, a saline and sugar solution, a peritoneal dialysate precursor solution, a hemodialysis precursor solution, a Ringer's solution concentrate, lactated Ringer's solution concentrate, and Hartmann's solution concentrate, a powder, a lyophilized agent, a crystalline solid, a salt in crystalline form, crystalline sodium chloride, crystalline sugar, a solid dialysate precursor, a concentrate for a hypotonic solution, a concentrate for an isotonic solution, a concentrate for a hypertonic solution, a saturated solution, an unsaturated solution, an at least partially liquid concentrate, and an at least partially solid concentrate.

418. The system of claim 414, wherein the dispenser includes a dispenser outlet, a dispenser outlet cover, and a dispenser lock assembly, the outlet cover in sealing relationship with the dispenser about the dispenser outlet when the lock assembly is in a locked state.

419. The system of claim 418, wherein the dispenser outlet cover interface includes a dispenser lock assembly actuation assembly and a pressure port in selective fluid communication with a negative pressure source.

420. The system of claim 414, wherein the reservoir singulation assembly includes a splitter assembly coupled to a splitter assembly actuator, the splitter assembly displaceable through a range from a raised position to an end of stroke position via the splitter assembly actuator.

421. The system of claim 414, wherein the filling assembly further comprises an irradiation assembly including a plurality of antimicrobial light emitter and the cartridge includes a barrier surrounding at least a portion of the dispensing sharp which is at least partially transparent to the antimicrobial light.

422. The system of claim 414, wherein the particulate inspection assembly includes a reservoir rest coupled to a rotary actuator.

423. The system of claim 422, wherein the system further comprises a controller in data communication with the rotary actuator, the controller configured to command displacement of the rotary actuator between preset pairs or rotational orientations defined in a motion profile.

424. The system of claim 414, wherein the particulate inspection system includes a contrasting pattern, an illuminator including at least one collimated light emitter and a polarizer, and at least one imager having a polarizing filter disposed opposite the contrasting pattern.

425. The system of claim 425, wherein the system further comprises a controller configured to receive image data from the at least one imager, process the image data, detect pixel clusters of interest in the image data, and determine classifications for the pixel clusters of interest.

426. The system of claim 425, wherein the controller is configured to supply a portion of the image data including at least the pixel clusters of interest to a convolutional neural network trained on images of reservoirs including known contents to determine the classifications.

427. The system of claim 414, wherein one of the at least one a first array of reservoir receptacles and one of the at least one a second array of reservoir receptacles are included in an outfeed drawer, the system further comprising a first lock for the outfeed drawer and a second lock for the outfeed drawer, the outfeed drawer displaceable to a partially open position when the first lock is disengaged and a fully opened position when the second lock is disengaged.
